(19) United States
(12) Patent Application Publication
(10) Pub. No.: US 2015/0261911 A1
(43) Pub. Date: Sep. 17, 2015

(54) CRYSTAL STRUCTURE

(71) Applicant: Heptares Therapeutics Limited, Welwyn Garden City (GB)

(72) Inventors: Andrea Bortolato, Welwyn Garden City (GB); Kin Yip Cheng, Welwyn Garden City (GB); Andrew Stephen Dore, Welwyn Garden City (GB); Kaspar Hollenstein, Welwyn Garden City (GB); Seyed Ali Jazayeri-Dezfuly, Welwyn Garden City (GB); James Kean, Welwyn Garden City (GB); Fiona Hamilton Marshall, Welwyn Garden City (GB)

(73) Assignee: Heptares Therapeutics Limited, Welwyn Garden City (GB)

(21) Appl. No.: 14/210,266

(22) Filed: Mar. 13, 2014

(51) Int. Cl. G06F 19/16; C07K 14/72; C07K 14/00

(52) U.S. Cl. CPC G06F 19/16; C07K 14/001; C07K 14/723; C07K 2299/00

(57) ABSTRACT

The invention provides a method of predicting a three dimensional structural representation of a target protein of unknown structure, or part thereof, comprising:
providing the coordinates of the human corticotropin-releasing factor receptor-1 (CRF1R) structure listed in Table A, Table B or Table C, optionally varied by a root mean square deviation of residue backbone atoms of not more than 4.383 Å, or selected coordinates thereof; and
predicting the three-dimensional structural representation of the target protein, or part thereof, by modelling the structural representation on all or the selected coordinates of the CRF1R structure.

The invention also provides the use of the CRF1R coordinates to select or design one or more binding partners of CRF1R.

### RELATED APPLICATIONS

This application claims the benefit under 35 U.S.C. §119(e) of U.S. Provisional Application Ser. No. 61/783,914, entitled “CRYSTAL STRUCTURE,” filed on Mar. 14, 2013, the entire disclosure of which is herein incorporated by reference in its entirety.

The present invention relates to protein crystal structures and their use in identifying protein binding partners and in protein structure determination. In particular, it relates to the crystal structure of a corticotropin-releasing factor receptor 1 (CRF1R) and uses thereof.

The listing or discussion of an apparently prior-published document in this specification should not necessarily be taken as an acknowledgement that the document is part of the state of the art or is common general knowledge.

G protein-coupled receptors (GPCRs) are integral membrane proteins mediating the signalling of a diverse set of ligands including neurotransmitters and metabolites. In humans, there are approximately 370 non-sensory receptors, representing the site of action for ˜30% of clinically used drugs. Activation of the receptor results in a conformational change propagated to the intracellular surface where the receptor interacts with heterotrimeric G proteins to regulate signalling to ion channels and enzyme pathways. GPCRs can also signal independently of G proteins through β-arrestin and are known to exist as dimers.

GPCRs can be classified into three classes (A, B and C) based on sequence similarity (1,2). Class B GPCRs include receptors for peptides such as secretin, glucagon, glucagon-like peptide, calcitonin and parathyroid peptide hormone and have been studied as drug targets in the treatment of various diseases, including diabetes, osteoporosis, depression and anxiety. They feature an N-terminal extracellular domain (ECD) involved in peptide-binding and a seven transmembrane-helices containing transmembrane domain (TMD) involved in signal transduction. Recently determined structures of Class A receptors have greatly advanced our understanding of the function of GPCRs at a molecular level (3). However, structural information on Class B receptors is currently limited to the ECD (4-9) and no structure of a Class B TMD, the main target for small-molecule drugs (10), has been determined to date.

The inventors have now solved the crystal structure of the TMD of the human CRF1R (11), a Class B GPCR essential for the stress-induced activation of the hypothalamic-pituitary-adrenal axis (12, 13), in complex with the non-peptide antagonist CP-376395 (14). The structure reveals significant differences to those of Class A receptors. The extracellular half of the receptor assumes a very open conformation, presumably to allow binding of the large ECD-peptide complex. Furthermore, in contrast to Class A GPCRs where the ligand-binding sites are located close to the extracellular boundaries of the receptors, in CRF1R the antagonist binds in a hydrophobic pocket located deep in the cytoplasmic half of the receptor. This structure provides new insight into the architecture of Class B GPCRs and should aid in the design of novel therapeutics.

The coordinates of the CRF1R can be utilised and manipulated in many different ways with wide ranging applications including the fitting of binding partners, homology modelling and structure solution, analysis of ligand interactions and drug discovery.

Accordingly, a first aspect of the invention provides a method of predicting a three dimensional structural representation of a target protein of unknown structure, or part thereof, comprising:
providing the coordinates of the human corticotropin-releasing factor receptor-1 (CRF1R) structure listed in Table A, Table B or Table C, optionally varied by a root mean square deviation of residue backbone atoms of not more than 4.383 Å, or selected coordinates thereof; and
predicting the three-dimensional structural representation of the target protein, or part thereof, by modelling the structural representation on all or the selected coordinates of the CRF1R structure.

By a ‘three dimensional structural representation’ we include a computer generated representation or a physical representation. Typically, in all aspects of the invention which feature a structural representation, the representation is computer generated. Computer representations can be generated or displayed by commercially available software programs. Examples of software programs include but are not limited to QUANTA (Accelrys COPYRIGHT, 2001, 2002), O (Jones et al., Acta Crystallogr. A47, pp. 110-119 (1991)), RIBBONS (Carson, J. Appl. Crystallogr., 24, pp. 9589-961 (1991)) and PyMol (The PyMol Molecular Graphics System, Schrödinger, LLC), which are incorporated herein by reference. Examples of representations include any of a wire-frame model, a chicken-wire model, a ball-and-stick model, a space-filling model, a stick model, a ribbon model, a snake model, an arrow and cylinder model, an electron density map or a molecular surface model. Certain software programs may also imbue these three dimensional representations with physico-chemical attributes which are known from the chemical composition of the molecule, such as residue charge, hydrophobicity, torsional and rotational degrees of freedom for the residue or segment, etc. Examples of software programs for calculating chemical energies are described below.

Typically, the coordinates of the CRF1R structure used in the invention are those listed in Table A or Table B or Table C, preferably those listed in Table C. However, it is appreciated that it is not necessary to have recourse to the original coordinates listed in Table A or Table B or Table C, and that any equivalent geometric representation derived from or obtained by reference to the original coordinates may be used.

Thus, for the avoidance of doubt, by ‘the coordinates of the CRF1R structure listed in Table A or Table B or Table C’, we include any equivalent representation wherein the original coordinates have been reparameterised in some way. For example, the coordinates in Table A or Table B or Table C may undergo any mathematical transformation known in the art, such as a geometric transformation, and the resulting transformed coordinates can be used. For example, the coordinates of Table A or Table B or Table C may be transposed to a different origin and/or axes or may be rotated about an axis. Furthermore, it is possible to use the coordinates to calculate the psi and phi backbone torsion angles (as displayed on a Ramachandran plot) and the chi sidechain torsion angles for each residue in the protein. These angles together with the corresponding bond lengths, enable the construction of a geometric representation of the protein which may be used based on the parameters of psi, phi and chi angles and bond lengths. Thus, while the coordinates used are typically those in Table A or Table B or Table C, the inventors recognise that any equivalent geometric representation of the CRF1R structure, based on the coordinates listed in Table A or Table B or Table C, may be used.

Additionally, it is appreciated that changing the number and/or positions of the ligand molecule of the Tables does not generally affect the usefulness of the coordinates in the aspects of the invention. Thus, it is also within the scope of the invention if the number and/or positions of ligand molecules of the coordinates of Table A or Table B or Table C is varied.

It will be appreciated that in all aspects of the invention which utilise the coordinates of the CRF1R, it is not necessary to utilise all the coordinates of Table A or Table B or Table C, but merely a portion of them, e.g. a set of coordinates representing atoms of particular interest in relation to a particular use. Such a portion of coordinates is referred to herein as ‘selected coordinates’.

By ‘selected coordinates’, we include at least 5, 10 or 20 non-hydrogen protein atoms of the Table A or Table B or Table C structure, more preferably at least 50, 100, 200, 300, 400, 500, 600, 700, 800 or 900 atoms and even more preferably at least 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300 or 3400 non-hydrogen atoms. Preferably the selected coordinates pertain to at least 5, 10, 20 or 30 different amino acid residues (i.e. at least one atom from 5, 10, 20 or 30 different residues may be present), more preferably at least 40, 50, 60, 70, 80 or 90 residues, and even more preferably at least 100, 150, 200, 250, 300 or 350 residues. Optionally, the selected coordinates may include one or more ligand atoms as set out in Table A or Table B or Table C. Alternatively, the selected coordinates may exclude one or more atoms of the ligand. Similarly, optionally, the selected coordinates may include one or more T4 lysozyme atoms as set out in Table A or Table B or Table C. Alternatively, the selected coordinates may exclude one or more T4 lysozyme atoms. Thus, it will be appreciated that the selected coordinates may include one or more ligand atoms and, optionally one or more T4 lysozyme atoms.

In one example, the selected coordinates may comprise atoms of one or more amino acid residues that contribute to the main chain or side chain atoms of a binding region of the CRF1R.

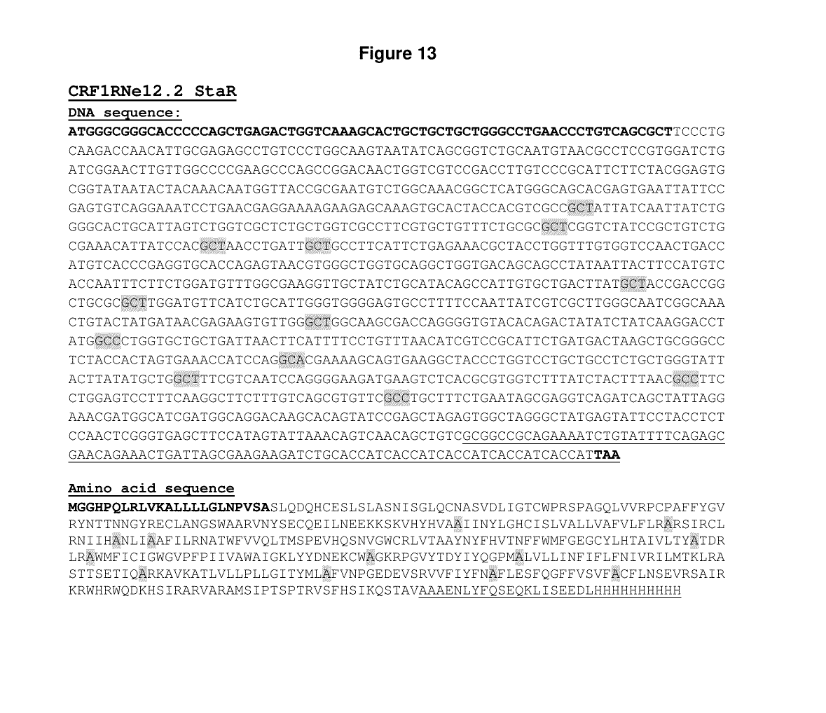

For example, amino acid residues contributing to a small organic molecule binding pocket include amino acid residues Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362 according to the numbering of the CRF1R sequence as set out in FIG. 13, all of which are within 7 Å of the CP-376395 ligand. Thus the selected coordinates may comprise one or more atoms from any one or more (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or 41) of amino acid residues Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, according to the numbering of the CRF1R sequence as set out in FIG. 13. Typically, coordinates of all of the atoms of the side chain are selected. Preferably, the selected coordinates comprise one or more atoms from any one or more (eg at least 2, 3, 4, 5, 6 or 7) of amino acids Phe 203, Met 206, Gly 210, Asn 283, Thr 316, Leu 323 and Tyr 327, according to the numbering of the CRF1R sequence as set out in FIG. 13, which include the nearest residues to the ligand.

In a further example, the selected coordinates may be from amino acid residues contributing to the peptide orthosteric binding site including amino acid acid residues Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355 according to the numbering of the CRF1R sequence as set out in FIG. 13. Thus the selected coordinates may comprise one or more atoms from any one or more (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or 41) of amino acid residues Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355, according to the numbering of the CRF1R sequence as set out in FIG. 13. Typically, coordinates of all of the atoms of the side chain are selected.

In a further example, the selected coordinates may comprise atoms of one or more amino acids involved in activation. For example, biochemical data suggests interaction of His 155 (2.50 Class B residue) and Glu 209 (3.50 Class B residue) play an essential role in activation (26-28). In the present structure, these two side chains are within hydrogen bonding distance (3.1 Å), forming a potentially important functional micro-switch. Thus, the selected coordinates may comprise atoms of one or both of amino acid residues His 155 (2.50) and Glu 209 (3.50) according to the numbering of the CRF1R as set out in FIG. 13. The numbers in brackets are based on a numbering system used to identify particular residues in Class B GPCRs (Wootten, D et al PNAS 2013, 201221585), wherein the position of each residue is described by two numbers. The first (1 to 7) corresponds to the TM helix in which the residue is located; the second indicates its position relative to a reference conserved residue in that helix, arbitrarily assigned to 50. The number decreases toward the N-terminus and increases towards the C-terminus.

The reference residues for CRF1R are:

### TM1: S130^{1.50}

### TM2: H155^{2.50}

### TM3: E209^{3.50}

### TM4: W236^{4.50}

### TM5: N283^{5.50}

### TM6: G324^{6.50}

### TM7: G356^{7.50}

In a further example, the selected coordinates may comprise atoms of amino acid residues belonging to the GWG×P motif found in TM4. Thus, the selected coordinates may comprise atoms of one or more of amino acid residues Gly 235, Trp 236, Gly 237 and Pro 239, according to the numbering of the CRF1R as set out in FIG. 13.

It is appreciated that the selected coordinates may comprise any atoms of particular interest including atoms mentioned in any one or more of the above examples, or as listed in Example 1 below.

It is appreciated that the selected coordinates may correspond to atoms from a particular structural region (e.g. helix and/or loop) of the CRF1R. By the helices and loop regions of the CRF1R we mean the following:

### Helix 1 Residues 115-143

### Helix 2 Residues 150-176

### Helix 3 Residues 185-218

### Helix 4 Residues 224-253

### Helix 5 Residues 269-298

### Helix 6 Residues 305-332

### Helix 7 Residues 339-368

### ICL1 Residues 144-149

### ECL1 Residues 177-184

### ICL2 Residues 219-223

### ECL2 Residues 254-268

### ICL3 Residues 299-304

### ECL3 Residues 333-338

However, it will be appreciated that there are different criteria for which residues are considered to be in a helical conformation depending on phi and psi angles. Moreover, when comparing the CRF1R to other structures, some residues may be missing in one or other of the structures and some residues may be considered helical in one structure but not the other. Further, the loop regions may be defined as amino acid structures that join alpha helices (as above) or may be defined as amino acid structures that are predicted to be outside of the membrane. Therefore the limits above are not to be construed as absolute, but rather may vary according to the criteria used. For the purposes of the comparisons set out below, we have used the definitions of helices and loops noted in Table 4 in Example 1.

Preferably, the selected coordinates include at least 2% or 5% C-α atoms, and more preferably at least 10% C-α atoms. Alternatively or additionally, the selected coordinates include at least 10% and more preferably at least 20% or 30% backbone atoms selected from any combination of the nitrogen, C-α, carbonyl C and carbonyl oxygen atoms.

It is appreciated that the coordinates of the CRF1R used in the invention may be optionally varied and a subset of the coordinates or the varied coordinates may be selected (and constitute selected coordinates). Indeed, such variation may be necessary in various aspects of the invention, for example in the modelling of protein structures and in the fitting of various binding partners to the CRF1R structure.

Protein structure variability and similarity is routinely expressed and measured by the root mean square deviation (rmsd), which measures the difference in positioning in space between two sets of atoms. The rmsd measures distance between equivalent atoms after their optimal superposition. The rmsd can be calculated over any sets of selected atoms including all atoms, over residue backbone atoms (i.e. the nitrogen-carbon-carbon backbone atoms of the protein amino acid residues), side chain atoms only or over C-α atoms only.

The least-squares algorithms used to calculate rmsd are well known in the art and include those described by Rossman and Argos (J Biol Chem, (1975) 250:7525), Kabsch (Acta Cryst (1976) A92:922; Acta Cryst (1978) A34:827-828), Hendrickson (Acta Cryst (1979) A35: 158), McLachan (J Mol Biol (1979) 128:49) and Kearsley (Acta Cryst (1989) A45:208). Both algorithms based on iteration in which one molecule is moved relative to the other, such as that described by Ferro and Hermans (Acta Cryst (1977) A33:345-347), and algorithms which locate the best fit directly (e.g. Kabsch's methods) may be used. Methods of comparing proteins structures are also discussed in Methods of Enzymology, vol 115: 397-420.

Typically, rmsd values are calculated using coordinate fitting computer programs and any suitable computer program known in the art may be used, for example MNYFIT (part of a collection of programs called COMPOSER, Sutcliffe et al (1987) Protein Eng 1:377-384). Other programs also include LSQMAN (Kleywegt & Jones (1994) A super position, CCP4/ESF-EACBM, Newsletter on Protein Crystallography, 31: 9-14), LSQKAB (Collaborative Computational Project 4. The CCP4 Suite: Programs for Protein Crystallography, Acta Cryst (1994) D50:760-763), QUANTA (Jones et al, Acta Cryst (1991) A47:110-119 and commercially available from Accelrys, San Diego, Calif.), Insight (Commercially available from Accelrys, San Diego, Calif.), Sybyl® (commercially available from Tripos, Inc., St Louis) and O (Jones et al., Acta Cryst (1991) A47:110-119).

In, for example, the programs LSQKAB and O, the user can define the residues in the two proteins that are to be paired for the purpose of the calculation. Alternatively, the pairing of residues can be determined by generating a sequence alignment of the two proteins as is well known in the art. The atomic coordinates can then be superimposed according to this alignment and an rmsd value calculated. The program Sequoia (Bruns et al (1999) J Mol Biol 288(3):427-439) performs the alignment of homologous protein sequences, and the superposition of homologous protein atomic coordinates. Once aligned, the rmsd can be calculated using programs detailed above. When the sequences are identical or highly similar, the structural alignment of proteins can be done manually or automatically as outlined above. Another approach would be to generate a superposition of protein atomic coordinates without considering the sequence.

We have conducted an rmsd analysis of residue backbone atoms (i.e. the nitrogen-carbon-carbon-oxygen backbone atoms of the protein) between the CRF1R structure and various known Class A GPCR structures (see Example 2). Similar scripts can be used to calculate rmsd values for any other selected coordinates. Rmsd values have been calculated on residue backbone atoms in the complete crystallised structure and on selected regions of interest as discussed below.

The Class A GPCR that had a structure most closely related to the present CRF1R structure was dopamine D3 receptor (PDB: 3PBL) (see Example 2). Conducting an rmsd analysis of residue backbone atoms between the whole of the present CRF1R (Table C) structure and the dopamine D3 receptor structure gave an rmsd value of 4.383 Å. The same analysis using the structure of CRF1R in Tables A or B in the alignment (233 and 223 corresponding amino acids respectively) gave respective rmsd values of 4.074 Å and 3.360 Å. Thus in one embodiment, the coordinates or selected coordinates of Table A or Table B or Table C may be optionally varied within an rmsd of residue backbone atoms (i.e. the nitrogen-carbon-carbon-oxygen backbone atoms of the protein) of not more than 4.383 Å. Preferably, the coordinates or selected coordinates are varied within an rmsd of residue backbone atoms of not more than 4.3 Å, 4.2 Å, 4.1 Å, 4.0 Å, 3.9 Å, 3.8 Å, 3.7 Å, 3.6 Å, 3.5 Å, 3.4 Å, 3.3 Å, 3.2 Å, 3.1 Å, 3.0 Å, 2.9 Å, 2.8 Å, 2.7 Å, 2.6 Å, 2.5 Å, 2.4 Å, 2.3 Å, 2.2 Å, 2.1 Å, 2.0 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å. When the coordinates or selected coordinates are from Table A, it is preferred if they are optionally varied within an rmsd of residue backbone atoms of not more than 4.074 Å, and when the coordinates or selected coordinates are from Table B, it is preferred if they are optionally varied within an rmsd of residue backbone atoms of not more than 3.360 Å.

We have conducted an rmsd analysis of residue backbone atoms between the present CRF1R structure (Table C) and the dopamine D3 receptor structure within the small organic molecule binding pocket (i.e. amino acid residues Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362). The rmsd value for residue backbone atoms is 1.676 Å. A similar analysis using the CRF1R structure of Table A or Table B gave respective rmsd values of 1.642 Å and 1.655 Å. Thus in an embodiment, where the coordinates or selected coordinates used in the invention are optionally varied within the small organic molecule binding pocket, they are varied within an rmsd of residue backbone atoms of not more than 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å.

We have conducted an rmsd analysis of residue backbone atoms between the present CRF1R structure (Table C) and the dopamine D3 receptor structure within the peptide orthosteric binding site (i.e. amino acid residues Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355). The rmsd value for residue backbone atoms is 4.242 Å. Thus in an embodiment, where the coordinates or selected coordinates used in the invention are optionally varied within the peptide orthosteric binding site, they are varied within an rmsd of residue backbone atoms of not more than 4.2 Å, 4.1 Å, 4.0 Å, 3.9 Å, 3.8 Å, 3.7 Å, 3.6 Å, 3.5 Å, 3.4 Å, 3.3 Å, 3.2 Å, 3.1 Å, 3.0 Å, 2.9 Å, 2.8 Å, 2.7 Å, 2.6 Å, 2.5 Å, 2.4 Å, 2.3 Å, 2.2 Å, 2.1 Å, 2.0 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å.

We have conducted an rmsd analysis of residue backbone atoms between the present CRF1R structure (Table C) and the dopamine D3 receptor structure within amino acids His 155 (2.50) and Glu 209 (3.50) which play an essential role in activation (26-28). The rmsd value for residue backbone atoms is 0.570 Å. The same analysis using the CFR1F structures of Table A or B gave rmsd values of 0.274 Å and 0.426 Å respectively. Thus in an embodiment, where the coordinates or selected coordinates used in the invention are optionally varied within amino acids His 155 (2.50) and Glu 209 (3.50), they are varied within an rmsd of residue backbone atoms of not more than 0.50 Å, 0.45 Å, 0.40 Å, 0.35 Å, 0.30 Å, 0.25 Å, 0.20 Å, 0.15 Å or 0.10 Å. When the coordinates, or selected coordinates are from Table A, it is preferred when they are optionally varied within amino acids His 155 (2.50) and Glu 209 (3.50) that they are optionally varied within an rmsd of residue backbone atoms of not more than 0.274 Å. When the coordinates, or selected coordinates are from Table B, it is preferred when they are optionally varied within amino acids His 155 (2.50) and Glu 209 (3.50) that they are optionally varied within an rmsd of residue backbone atoms of not more than 0.426 Å.

We have conducted an rmsd analysis of residue backbone atoms between the present CRF1R structure (Table C) and the dopamine D3 receptor structure within GWG×P motif found in TM4 (i.e. amino acid residues Gly 235, Trp 236, Gly 237 and Pro 239, according to the numbering of the CRF1R as set out in FIG. 13). The rmsd value for residue backbone atoms is 0.839 Å. There was no significant variation in the analysis when done using the CRF1R structures of Tables A and B. Thus in an embodiment, where the coordinates or selected coordinates used in the invention are optionally varied within amino acids of the GWG×P motif, they are varied within an rmsd of residue backbone atoms of not more than 0.8 Å, 0.7 Å, 0.6 Å, 0.5 Å, 0.4 Å, 0.3 Å, 0.2 Å or 0.1 Å.

We have conducted an rmsd analysis of residue backbone atoms between the present CRF1R structure (Table C) and various Class A GPCR structures within the common transmembrane region (i.e. amino acid residues 119-143, 150-176, 186-218, 227-247, 269-294, 312-332 and 343-365 according to the numbering of the CRF1R as set out in FIG. 13; corresponding to Class A Ballesteros-Weinstein residues 1.35-1.59, 2.38-2.64, 3.23-3.55, 4.41-4.61, 5.40-5.65, 6.33-6.53 and 7.33-7.55). The Class A GPCRs that have a structure most closely related to the present CRF1R structure in the common TM region are the adenosine A2a receptor (PDB ID 2YDV) and the dopamine D3 receptor (PDB ID 3PBL). In each case, the rmsd value for residue backbone atoms is 2.7 Å. Thus in an embodiment, where the coordinates or selected coordinates used in the invention are optionally varied within amino acids of the common transmembrane region, they are varied within an rmsd of residue backbone atoms of not more than 2.70 Å (such as not more than 2.6 Å, 2.5 Å, 2.4 Å, 2.3 Å, 2.2 Å, 2.1 Å, 2.0 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å).

We have conducted an rmsd analysis of residue backbone atoms between the present CRF1R structure (Table C) and various Class A GPCR structures within transmembrane 1 (TM1) (i.e. amino acid residues 119-143 according to the numbering of the CRF1R as set out in FIG. 13; corresponding to Class A Ballesteros-Weinstein residues 1.35-1.59). The analysis was done following a global superposition using a core TM region shared by all receptors as described in Table 4. The Class A GPCR that has a structure most closely related to the present CRF1R structure in TM1 is the histamine H1 receptor (PDB ID 3RZE). The rmsd value for residue backbone atoms is 1.8 Å. Thus in an embodiment, where the coordinates or selected coordinates used in the invention are optionally varied within amino acids of TM1, they are varied within an rmsd of residue backbone atoms of not more than 1.8 Å (such as not more than 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å). When the rmsd analysis was conducted following a local superposition of the individual TM region, the Class A GPCR that has a structure most closely related to the present CRF1R structure in TM1 is the adenosine A2a receptor (PDB ID 3PWH), and the rmsd for residue backbone atoms is 0.5 Å. Thus, especially when the coordinates are only selected from TM1, it is preferred if they are optionally varied within an rmsd of not more than 0.5 Å (such as not more than 0.45 Å, 0.40 Å, 0.35 Å, 0.30 Å, 0.25 Å, 0.20 Å, 0.15 Å or 0.10 Å).

We have conducted an rmsd analysis of residue backbone atoms between the present CRF1R structure (Table C) and various Class A GPCR structures within transmembrane 2 (TM2) (i.e. amino acid residues 150-176 according to the numbering of the CRF1R as set out in FIG. 13; corresponding to Class A Ballesteros-Weinstein residues 2.38-2.64). The analysis was done following a global superposition using a core TM region shared by all receptors as described in Table 4. The Class A GPCR that has a structure most closely related to the present CRF1R structure in TM2 is the 1-phosphate S1P1 receptor receptor (PDB ID 3V2Y). The rmsd value for residue backbone atoms is 2.0 Å. Thus in an embodiment, where the coordinates or selected coordinates used in the invention are optionally varied within amino acids of TM2, they are varied within an rmsd of residue backbone atoms of not more than 2.0 Å (such as not more than 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å). When the rmsd analysis was conducted following a local superposition of the individual TM region, the Class A GPCR that has a structure most closely related to the present CRF1R structure in TM2 is the S1P₁ receptor (PDB ID 3V2Y), and the rmsd for residue backbone atoms is 0.7 Å. Thus, especially when the coordinates are only selected from TM2, it is preferred if they are optionally varied within an rmsd of not more than 0.7 Å (such as not more than 0.65 Å, 0.60 Å, 0.55 Å, 0.40 Å, 0.45 Å, 0.40 Å, 0.35 Å, 0.30 Å, 0.25 Å, 0.20 Å, 0.15 Å or 0.10 Å)

We have conducted an rmsd analysis of residue backbone atoms between the present CRF1R structure (Table C) and various Class A GPCR structures within transmembrane 3 (TM3) (i.e. amino acid residues 186-218 according to the numbering of the CRF1R as set out in FIG. 13; corresponding to Class A Ballesteros-Weinstein residues 3.23-3.55). The analysis was done following a global superposition using a core TM region shared by all receptors as described in Table 4. The Class A GPCR that has a structure most closely related to the present CRF1R structure in TM3 is the neurotensin receptor NTSR1 (PDB ID 4GRV). The rmsd value for residue backbone atoms is 1.2 Å. Thus in an embodiment, where the coordinates or selected coordinates used in the invention are optionally varied within amino acids of TM3, they are varied within an rmsd of residue backbone atoms of not more than 1.2 Å (such as not more than 1.1 Å, 1.0 Å, 0.9 Å, 0.8 Å, 0.7 Å, 0.6 Å, 0.5 Å, 0.4 Å, 0.3 Å, 0.2 Å or 0.1 Å). When the rmsd analysis was conducted following a local superposition of the individual TM region, the Class A GPCRs that have a structure most closely related to the present CRF1R structure in TM3 are the beta-2 adrenergic receptor (PDB ID 3SN6), the dopamine D3 receptor (PDB ID 3PBL) and the muscarinic M₂ receptor (PDB ID 3UON), and in each case the rmsd for residue backbone atoms is 0.6 Å. Thus, especially when the coordinates are only selected from TM3, it is preferred if they are optionally varied within an rmsd of not more than 0.60 Å (such as not more than 0.55 Å, 0.50 Å, 0.45 Å, 0.40 Å, 0.35 Å, 0.30 Å, 0.25 Å, 0.20 Å, 0.15 Å or 0.10 Å)

We have conducted an rmsd analysis of residue backbone atoms between the present CRF1R structure (Table C) and various Class A GPCR structures within transmembrane 4 (TM4) (i.e. amino acid residues 227-247 according to the numbering of the CRF1R as set out in FIG. 13; corresponding to Class A Ballesteros-Weinstein residues 4.41-4.61). The analysis was done following a global superposition using a core TM region shared by all receptors as described in Table 4. The Class A GPCRs that have a structure most closely related to the present CRF1R structure in TM4 are the β2-adrenergic receptor (PDB ID 2RH1) and the muscarinic M2 receptor (PDB ID 3UON). In each case, the rmsd value for residue backbone atoms is 2.5 Å. Thus in an embodiment, where the coordinates or selected coordinates used in the invention are optionally varied within amino acids of TM4, they are varied within an rmsd of residue backbone atoms of not more than 2.5 Å (such as not more than 2.4 Å, 2.3 Å, 2.2 Å, 2.1 Å, 2.0 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å). When the rmsd analysis was conducted following a local superposition of the individual TM region, the Class A GPCR that has a structure most closely related to the present CRF1R structure in TM4 is the muscarinic M2 receptor (PDB ID 3UON), and the rmsd for residue backbone atoms is 1.4 Å. Thus, especially when the coordinates are only selected from TM4, it is preferred if they are optionally varied within an rmsd of not more than 1.4 Å (such as not more than 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å)

We have conducted an rmsd analysis of residue backbone atoms between the present CRF1R structure (Table C) and various Class A GPCR structures within transmembrane 5 (TM5) (i.e. amino acid residues 269-294 according to the numbering of the CRF1R as set out in FIG. 13; corresponding to Class A Ballesteros-Weinstein residues 5.40-5.65). The analysis was done following a global superposition using a core TM region shared by all receptors as described in Table 4. The Class A GPCR that has a structure most closely related to the present CRF1R structure in TM5 is the dopamine D3 receptor (PDB ID 3PBL). The rmsd value for residue backbone atoms is 2.2 Å. Thus in an embodiment, where the coordinates or selected coordinates used in the invention are optionally varied within amino acids of TM5, they are varied within an rmsd of residue backbone atoms of not more than 2.2 Å (such as not more than 2.2 Å, 2.1 Å, 2.0 Å, 1.9 Å, 1.8, Å 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å). When the rmsd analysis was conducted following a local superposition of the individual TM region, the Class A GPCR that has a structure most closely related to the present CRF1R structure in TM5 is the S1P₁ receptor (PDB ID 3V2Y), and the rmsd for residue backbone atoms is 0.7 Å. Thus, especially when the coordinates are only selected from TM5, it is preferred if they are optionally varied within an rmsd of not more than 0.7 Å (such as not more than 0.65 Å, 0.60 Å, 0.55 Å, 0.40 Å, 0.45 Å, 0.40 Å, 0.35 Å, 0.30 Å, 0.25 Å, 0.20 Å, 0.15 Å or 0.10 Å)

We have conducted an rmsd analysis of residue backbone atoms between the present CRF1R structure (Table C) and various Class A GPCR structures within transmembrane 6 (TM6) (i.e. amino acid residues 312-332 according to the numbering of the CRF1R as set out in FIG. 13; corresponding to Class A Ballesteros-Weinstein residues 6.33-6.53). The analysis was done following a global superposition using a core TM region shared by all receptors as described in Table 4. The Class A GPCR that has a structure most closely related to the present CRF1R structure in TM6 is the adenosine A2a receptor (PDB ID 2YDV). The rmsd value for residue backbone atoms is 2.4 Å. Thus in an embodiment, where the coordinates or selected coordinates used in the invention are optionally varied within amino acids of TM6, they are varied within an rmsd of residue backbone atoms of not more than 2.4 Å (such as not more than 2.3 Å, 2.2 Å, 2.1 Å, 2.0 Å, 1.9 Å, 1.8, Å 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å). When the rmsd analysis was conducted following a local superposition of the individual TM region, the Class A GPCRs that have a structure most closely related to the present CRF1R structure in TM6 are the dopamine D3 receptor (PDB ID 3PBL), the muscarinic M₂ receptor (PDB ID 3UON), the kappa opioid receptor (PDB ID 4DJH) and the mu opioid receptor (PDB ID 4DKL), and in each case the rmsd for residue backbone atoms is 1.1 Å. Thus, especially when the coordinates are only selected from TM6, it is preferred if they are optionally varied within an rmsd of not more than 1.1 Å (such as not more than 1.0 Å, 0.9 Å, 0.8 Å, 0.7 Å, 0.6 Å, 0.5 Å, 0.4 Å, 0.3 Å, 0.2 Å or 0.1 Å)

We have conducted an rmsd analysis of residue backbone atoms between the present CRF1R structure (Table C) and various Class A GPCR structures within transmembrane 7 (TM7) (i.e. amino acid residues 343-365 according to the numbering of the CRF1R as set out in FIG. 13; corresponding to Class A Ballesteros-Weinstein residues 7.33-7.55). The analysis was done following a global superposition using a core TM region shared by all receptors as described in Table 4. The Class A GPCR that has a structure most closely related to the present CRF1R structure in TM7 is the adenosine A2a receptor (PDB ID 2YDV). The rmsd value for residue backbone atoms is 4.3 Å. Thus in an embodiment, where the coordinates or selected coordinates used in the invention are optionally varied within amino acids of TM7, they are varied within an rmsd of residue backbone atoms of not more than 4.3 Å (such as not more than 4.2 Å, 4.1 Å, 4.0 Å, 3.9 Å, 3.8 Å, 3.7 Å, 3.6 Å, 3.5 Å, 3.4 Å, 3.3 Å, 3.2 Å, 3.1 Å, 3.0 Å, 2.9 Å, 2.8 Å, 2.7 Å, 2.6 Å, 2.5 Å, 2.4 Å, 2.3 Å, 2.2 Å, 2.1 Å, 2.0 Å, 1.9 Å, 1.8, Å 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å or 0.1 Å). When the rmsd analysis was conducted following a local superposition of the individual TM region, the Class A GPCR that has a structure most closely related to the present CRF1R structure in TM7 is the muscarinic M₂ receptor (PDB ID 3UON), and the rmsd for residue backbone atoms is 1.0 Å. Thus, especially when the coordinates are only selected from TM7, it is preferred if they are optionally varied within an rmsd of not more than 1.0 Å (such as not more than 0.95 Å, 0.90 Å, 0.85 Å, 0.80 Å, 0.75 Å, 0.70 Å, 0.65 Å, 0.60 Å, 0.55 Å, 0.40 Å, 0.45 Å, 0.40 Å, 0.35 Å, 0.30 Å, 0.25 Å, 0.20 Å, 0.15 Å or 0.10 Å)

In this aspect of the invention, the coordinates of the CRF1R structure are used to predict a three dimensional representation of a target protein of unknown structure, or part thereof, by modelling. By “modelling”, we mean the prediction of structures using computer-assisted or other de novo prediction of structure, based upon manipulation of the coordinate data from Table A or Table B or Table C, or selected coordinates thereof.

The target protein may be any protein that shares sufficient sequence identity to the human CRF1R such that its structure can be modelled by using the CRF1R coordinates of Table A or Table B or Table C. It will be appreciated that if a structural representation of only a part of the target protein is being modelled, for example a particular domain, the target protein only has to share sufficient sequence identity to the CRF1R over that part.

It has been shown for soluble protein domains that their three dimensional structure is broadly conserved above 20% amino acid sequence identity and well conserved above 30% identity, with the level of structural conservation increasing as amino acid sequence identity increases up to 100% (Ginalski, K. Curr Op Struc Biol (2006) 16, 172-177). Thus, it is preferred if the target protein, or part thereof, shares at least 20% amino acid sequence identity with the human CRF1R sequence provided in FIG. 13, and more preferably at least 30%, 40%, 50%, 60%, 70%, 80% or 90% sequence identity, and yet more preferably at least 95% or 99% sequence identity. It is particularly preferred if the target protein, or part thereof, shares any of the specified sequence identities with the TM region (i.e. TMs 1-7) of the human CRF1R sequence provided in FIG. 13.

It will be appreciated therefore that the target protein may be an CRF1R analogue or homologue.

Analogues are defined as proteins with similar three-dimensional structures and/or functions with little evidence of a common ancestor at a sequence level.

Homologues are proteins with evidence of a common ancestor, i.e. likely to be the result of evolutionary divergence and are divided into remote, medium and close sub-divisions based on the degree (usually expressed as a percentage) of sequence identity.

By a human CRF1R homologue, we include a protein with at least 20%, 25%, 30%, 35%, 40%, 45% or at least 50% amino acid sequence identity with the sequence of CRF1R provided in FIG. 13, preferably at least 55%, 60%, 65%, 70%, 75% or 80% amino acid sequence identity and more preferably 85%, 90%, 95% or 99% amino acid sequence identity. This includes polymorphic forms of CRF1R receptors, e.g. mutants and CRF1R receptors from other species as well as other corticoptropin releasing factor receptors such as CRF2R. Thus an CRF1R homologue would include a human CRF2R.

Sequence identity may be measured by the use of algorithms such as BLAST or PSI-BLAST (Altschul et al, NAR (1997), 25, 3389-3402) or methods based on Hidden Markov Models (Eddy S et al, J Comput Biol (1995) Spring 2 (1) 9-23). Typically, the percent sequence identity between two polypeptides may be determined using any suitable computer program, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al., 1994). The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

In one embodiment the target protein is an integral membrane protein. By “integral membrane protein” we mean a protein that is normally integrated into the membrane and can only be removed using detergents, non-polar solvents or denaturing agents that physically disrupt the lipid bilayer. Examples include receptors such as GPCRs, the T-cell receptor complex and growth factor receptors; transmembrane ion channels such as ligand-gated and voltage gated channels; transmembrane transporters such as neurotransmitter transporters; enzymes; carrier proteins; and ion pumps.

The amino acid sequences (and the nucleotide sequences of the cDNAs which encode them) of many membrane proteins are readily available, for example by reference to GenBank. For example, Foord et al supra gives the human gene symbols and human, mouse and rat gene IDs from Entrez Gene (http://www.ncbi.nlm.nih.gov/entrez) for GPCRs. It should be noted, also, that because the sequence of the human genome is substantially complete, the amino acid sequences of human membrane proteins can be deduced therefrom.

In a preferred embodiment, the target protein is a GPCR. GPCRs are well known in the art and include those listed in Hopkins & Groom supra. In addition, the International Union of Pharmacology produce a list of GPCRs (Foord et al (2005) Pharmacol. Rev. 57, 279-288, incorporated herein by reference and this list is periodically updated at http://www.iuphar-db.org/GPCR/ReceptorFamiliesForward). It will be noted that GPCRs are divided into different classes, principally based on their amino acid sequence similarities. They are also divided into families by reference to the natural ligands to which they bind. All GPCRs are included in the scope of the invention and their structure may be modelled by using the coordinates of the CRF1R. CRF1R is a Class B GPCR (sometimes known as Class 2 or Family B GPCRs which terms are used interchangeably).

In a particularly preferred embodiment, the target protein is a Class B GPCR, including a Class B GPCR in the secretin class such as any of glucagon-like peptide 1 receptor (GLP1R), glucagon-like peptide 2 receptor (GLP2R), calcitonin receptor (CT), amylin/CGRP receptor (AMY₁α), amylin receptor (AMY₂α), amylin/CGRP receptor (AMY₃α), CGRP/adrenomedullin receptor (CGRP₁α), adrenomedullin/CGRP receptor (AM₁α), adrenomedullin/CGRP receptor (AM₂α receptor), corticotropin releasing factor receptor (CRF₁), urocortins receptor (CRF₂), growth hormone releasing hormone receptor (GHRH), gastric inhibitory polypeptide receptor (GIP), glucagon receptor, secretin receptor, TIP-39 receptor (PTH2), parathyroid hormone receptor (PTH1), VIP/PACAP receptor (VPAC₁), PACAP receptor (PAC₂), and VIP/PACAP receptor (VPAC₂). Alternatively, the target protein is a Class B GPCR in the adhesion class such as any of Brain-specific angiogenesis inhibitor 1 (BAI1), Brain-specific angiogenesis inhibitor 2 (BAI2), Brain-specific angiogenesis inhibitor 1 (BAI3), CD97, Cadherin EGF LAG seven-pass G-type receptor 1 (CELSR1), Cadherin EGF LAG seven-pass G-type receptor 2 (CELSR2), Cadherin EGF LAG seven-pass G-type receptor 3 (CELSR3), EGF latrophilin seven transmembrane domain containing 1 (ELTD1), EGF-like module receptor 1 (EMR1), EGF-like module receptor 2 (EMR2), EGF-like module receptor 3 (EMR3), EGF-like module-containing mucin-like hormone receptor-like 4 (EMR4P), G protein coupled receptor 56 (GPR56), G protein coupled receptor 64 (GPR64), G protein coupled receptor 97 (GPR97), G protein coupled receptor 98 (GPR98), G protein coupled receptors from 110 to 116 (GPR110-116), G protein coupled receptors from 123 to 126 (GPR123-126), G protein coupled receptor 128 (GPR128), G protein coupled receptor 133 (GPR133), G protein coupled receptor 144 (GPR144), G protein coupled receptor 157 (GPR157) and Latrophilin 1 to 3 (LPHN1-3).

Although the target protein may be derived from any source, it is particularly preferred if it is from a eukaryotic source. It is particularly preferred if it is derived from a vertebrate source such as a mammal. It is particularly preferred if the target protein is derived from rat, mouse, rabbit or dog or non-human primate or man.

Typically, modelling a structural representation of a target is done by homology modelling whereby homologous regions between the CRF1R and the target protein are matched and the coordinate data of the CRF1R used to predict a structural representation of the target protein.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

Typically, the method involves comparing the amino acid sequences of CRF1R with a target protein by aligning the amino acid sequences. Amino acids in the sequences are then compared and groups of amino acids that are homologous (conveniently referred to as “corresponding regions”) are grouped together. This method detects conserved regions of the polypeptides and accounts for amino acid insertions or deletions.

Homology between amino acid sequences can be determined using commercially available algorithms known in the art. For example, the programs BLAST, gapped BLAST, BLASTN, PSI-BLAST, BLAST 2 and WU-BLAST (provided by the National Center for Biotechnology Information) can be used to align homologous regions of two, or more, amino acid sequences. These may be used with default parameters to determine the degree of homology between the amino acid sequence of the CRF1R and other target proteins which are to be modelled.

Preferred for use according to the present invention is the WU-BLAST (Washington University BLAST) version 2.0 software. WU-BLAST version 2.0 executable programs for several UNIX platforms can be downloaded from ftp://blast. wustl. edu/blast/executables. This program is based on WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Once the amino acid sequences of CRF1R and the target protein of unknown structure have been aligned, the structures of the conserved amino acids in the structural representation of the CRF1R may be transferred to the corresponding amino acids of the target protein. For example, a tyrosine in the amino acid sequence of CRF1R may be replaced by a phenylalanine, the corresponding homologous amino acid in the amino acid sequence of the target protein.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization. Typically, the predicted three dimensional structural representation will be one in which favourable interactions are formed within the target protein and/or so that a low energy conformation is formed (“High resolution structure prediction and the crystallographic phase problem” Qian et al (2007) Nature 450; 259-264; “State of the art in studying protein folding and protein structure production using molecular dynamics methods” Lee et al (2001) J of Mol Graph & Modelling 19(1): 146-149).

Whereas it is preferred to base homology modelling on homologous amino acid sequences, it is appreciated that some proteins have low sequence identity (e.g. Class B and C GPCRs) and at the same time are very similar in structure. Therefore, where at least part of the structure of the target protein is known, homologous regions can also be identified by comparing structures directly.

Homology modelling as such is a technique well known in the art (see e.g. Greer, (Science, Vol. 228, (1985), 1055), and Blundell et al (Eur. J. Biochem, Vol. 172, (1988), 513)). The techniques described in these references, as well as other homology modelling techniques generally available in the art, may be used in performing the present invention.

Typically, homology modelling is performed using computer programs, for example SWISS-MODEL available through the Swiss Institute for Bioinformatics in Geneva, Switzerland; WHATIF available on EMBL servers; Schnare et al. (1996) J. Mol. Biol, 256: 701-719; Blundell et al. (1987) Nature 326: 347-352; Fetrow and Bryant (1993) Bio/Technology 11:479-484; Greer (1991) Methods in Enzymology 202: 239-252; and Johnson et al (1994) Crit. Rev. Biochem. Mol Biol. 29:1-68. An example of homology modelling is described in Szklarz G. D (1997) Life Sci. 61: 2507-2520.

Thus, in an embodiment of the first aspect of the invention, the method further comprises aligning the amino acid sequence of the target protein of unknown structure with the amino acid sequence of CRF1R listed in FIG. 13 to match homologous regions of the amino acid sequences, and subsequently modelling the structural representation of the target protein by modelling the structural representation of the matched homologous regions of the target protein on the corresponding regions of the CRF1R to obtain a three dimensional structural representation for the target protein that substantially preserves the structural representation of the matched homologous regions.

The invention therefore provides a method of predicting a three dimensional structural representation of a target protein of unknown structure, or part thereof, comprising:
providing the coordinates of the CRF1R structure listed in Table A, Table B or Table C, optionally varied by a root mean square deviation of residue backbone atoms of not more than 4.383 Å, or selected coordinates thereof;
aligning the amino acid sequence of a target protein of unknown structure or part thereof with the amino acid sequence of CRF1R listed in FIG. 13 or part thereof to match homologous regions of the amino acid sequences;
modelling the structure of the matched homologous regions of the target protein on the corresponding regions of the CRF1R structure as defined by Table A or Table B or Table C, optionally varied by a root mean square deviation of residue backbone atoms of not more than 4.383 Å, or selected coordinates thereof; and
predicting a three dimensional structural representation for the target protein which substantially preserves the structure of the matched homologous regions.

The coordinate data of Table A or Table B or Table C, or selected coordinates thereof, will be particularly advantageous for homology modelling of other GPCRs. For example, since the protein sequence of CRF1R and another GPCR can be aligned relative to each other, it is possible to predict structural representations of the structures of other GPCRs, particularly in the regions of the transmembrane helices and ligand binding region, using the CRF1R coordinates.

The coordinate data of the CRF1R can also be used to predict the structure of target proteins where X-ray diffraction data or NMR spectroscopic data of the protein has been generated and requires interpretation in order to provide a structure.

A second aspect of the invention provides a method of predicting the three dimensional structural representation of a target protein of unknown structure, or part thereof, comprising: providing the coordinates of the human corticotropin-releasing factor receptor-1 (CRF1R) structure listed in Table A, Table B or Table C, optionally varied by a root mean square deviation of residue backbone atoms of not more than 4.383 Å, or selected coordinates thereof; and either (a) positioning the coordinates in the crystal unit cell of the protein so as to predict its structural representation, or (b) manipulating the coordinates to assign, or account for, peaks in NMR spectra.

Thus, where X-ray crystallographic or NMR spectroscopic data is provided for a target protein of unknown structure, the coordinate data of Table A or Table B or Table C may be used to interpret that data to predict a likely structure using techniques well known in the art including phasing, in the case of X-ray crystallography, and assisting peak assignments in the case of NMR spectra.

A three dimensional structural representation of any part of any target protein that is sufficiently similar to any portion of the CRF1R can be predicted by this method. Typically, the target protein or part thereof has at least 20% amino acid sequence identity with any portion of CRF1R, such as at least 30% amino acid sequence identity or at least 40% or 50% or 60% or 70% or 80% or 90% sequence identity. For example, the coordinates may be used to predict the three-dimensional representations of other crystal forms of CRF1R, other CRF1R receptors, CRF1R mutants or co-complexes of a CRF1R receptor. Other suitable target proteins are as defined with respect to the first aspect of the invention.

One method that may be employed for these purposes is molecular replacement which is well known in the art and described, for example, in Evans & McCoy (Acta Cryst, 2008, D64:1-10), McCoy (Acta Cryst, 2007, D63:32-42) and McCoy et al (J of App Cryst, 2007, 40:658-674). Molecular replacement enables the solution of the crystallographic phase problem by providing initial estimates of the phases of the new structure from a previously known structure, as opposed to the other major methods for solving the phase problem, i.e. experimental methods (which measure the phase from isomorphous or anomalous differences) or direct methods (which use mathematical relationships between reflection triplets and quartets to bootstrap a phase set for all reflections from phases for a small or random ‘seed’ set of reflections.) Compared to molecular replacement, such methods are time consuming and generally hinder the solution of crystal structures. Thus molecular replacement provides an accurate structural form for an unknown crystal more quickly and efficiently than attempting to determine such information ab initio.

Accordingly, the invention involves generating a preliminary model of a target protein whose structure coordinates are unknown, by orienting and positioning the relevant portion of the CRF1R according to Table A or Table B or Table C within the unit cell of a crystal of the target protein so as best to account for the observed X-ray diffraction pattern of the crystal of the target protein. Phases can be calculated from this model and combined with the observed X-ray diffraction pattern amplitudes to generate an electron density map of the target protein's structure. This, in turn, can be subjected to any well-known model building and structure refinement techniques to provide a final, accurate structural representation of the target protein (E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)).

Thus the invention includes a method of predicting a three dimensional structural representation of a target protein of unknown structure, or part thereof, comprising: providing the coordinates of the CRF1R structure, optionally varied by a root mean square deviation of residue backbone atoms of not more than 4.383 Å, or selected coordinates thereof; providing an X-ray diffraction pattern of the target protein; and using the coordinates to predict at least part of the structure coordinates of the target protein.

In an embodiment, the X-ray diffraction pattern of the target protein is provided by crystallising the target protein unknown structure; and generating an X-ray diffraction pattern from the crystallised target protein. Thus, the invention also provides a method of predicting a three dimensional structural representation of a target protein of unknown structure comprising the steps of (a) crystallising the target protein; (b) generating an X-ray diffraction pattern from the crystallised target protein; (c) applying the coordinates of the CRF1R structure, optionally varied by a root mean square deviation of residue backbone atoms of not more than 4.383 Å, or selected coordinates thereof, to the X-ray diffraction pattern to generate a three-dimensional electron density map of the target protein, or part thereof; and (d) predicting a three dimensional structural representation of the target protein from the three-dimensional electron density map.

Examples of computer programs known in the art for performing molecular replacement include CNX (Brunger A T.; Adams P. D.; Rice L. M., Current Opinion in Structural Biology, Volume 8, Issue 5, October 1998, Pages 606-611 (also commercially available from Accelrys San Diego, Calif.), MOLREP (A. Vagin, A. Teplyakov, MOLREP: an automated program for molecular replacement, J Appl Cryst (1997) 30, 1022-1025, part of the CCP4 suite), AMoRe (Navaza, J. (1994). AMoRe: an automated package for molecular replacement. Acta Cryst A50, 157-163), or PHASER (part of the CCP4 suite).

Preferred selected coordinates of the CRF1R are as defined above with respect to the first aspect of the invention.

The invention may also be used to assign peaks of NMR spectra of target proteins, by manipulation of the data of Table A or Table B or Table C (J Magn Reson (2002) 157(1): 119-23).

The coordinates of the CRF1R structure, optionally varied by a root mean square deviation of residue backbone atoms of not more than 4.383 Å, or selected coordinates thereof may be used in the provision, design, modification or analysis of binding partners of CRF1R. Such a use will be important in drug design.

By CRF1R we mean any CRF1R which has at least 75% sequence identity with human CRF1R as well as CRF1R receptors from other species and mutants thereof. Preferably, the CRF1R has at least 80% amino acid sequence identity to human CRF1R, and more preferably at least 85%, 90%, 95% or 99% amino acid sequence identity.

By “binding partner” we mean any molecule that binds to a CRF1R. Preferably, the molecule binds selectively to the CRF1R. For example, it is preferred if the binding partner has a K_{d} value (dissociation constant) which is at least five or ten times lower (i.e. higher affinity) than for at least one other GPCR, and preferably more than 100 or 500 times lower. More preferably, the binding partner of a CRF1R has a K_{d} value more than 1000 or 5000 times lower than for at least one other GPCR. However, it will be appreciated that the limits will vary dependent upon the nature of the binding partner.

Thus, typically, for small molecule binding partners, the binding partner typically has a K_{d} value which is at least 10 times or 50 times or 100 times lower than for at least one other GPCR. Typically, for antibody binding partners, the binding partner typically has a K_{d} value which is at least 500 or 1000 times lower than for at least one other GPCR.

K_{d} values can be determined readily using methods well known in the art and as described, for example, below.

At equilibrium Kd=[R][L]/[RL]

where the terms in brackets represent the concentration of
Receptor-ligand complexes [RL],
unbound receptor [R], and
unbound (“free”) ligand [L].

In order to determine the K_{d} the value of these terms must be known. Since the concentration of receptor is not usually known then the Hill-Langmuir equation is used where Fractional occupancy=[L]/[L]+K_{d}.

In order to experimentally determine a K_{d} then, the concentration of free ligand and bound ligand at equilibrium must be known. Typically, this can be done by using a radio-labelled or fluorescently labelled ligand which is incubated with the receptor (present in whole cells or homogenised membranes) until equilibrium is reached. The amount of free ligand vs bound ligand must then be determined by separating the signal from bound vs free ligand. In the case of a radioligand this can be done by centrifugation or filtration to separate bound ligand present on whole cells or membranes from free ligand in solution. Alternatively a scintillation proximity assay is used. In this assay the receptor (in membranes) is bound to a bead containing scintillant and a signal is only detected by the proximity of the radioligand bound to the receptor immobilised on the bead.

The binding partner may be any of a polypeptide; an anticalin; a peptide; an antibody; a chimeric antibody; a single chain antibody; an aptamer; a darpin; a Fab, F(ab′)₂, Fv, ScFv or dAb antibody fragment; a small molecule; a natural product; an affibody; a peptidomimetic; a nucleic acid; a peptide nucleic acid molecule; a lipid; a carbohydrate; a protein based on a modular framework including ankyrin repeat proteins, armadillo repeat proteins, leucine rich proteins, tetrariopeptide repeat proteins or Designed Ankyrin Repeat Proteins (DARPins); a protein based on lipocalin or fibronectin domains or Affilin scaffolds based on either human gamma crystalline or human ubiquitin; a G protein; an RGS protein; an arrestin; a GPCR kinase; a receptor tyrosine kinase; a RAMP; a NSF; a GPCR; an NMDA receptor subunit NR1 or NR2a; calcyon; or a fragment or derivative thereof that binds to CRF1R. Typically, the binding partner is a small molecule.

It will be appreciated that the coordinates of the invention will also be useful in the analysis of solvent and ion interactions with a CRF1R, which are important factors in drug design. Thus the binding partner may be a solvent molecule, for example water or acetonitrile, or an ion, for example a sodium ion or a protein.

It is particularly preferred if the binding partner is a small molecule with a molecule weight less than 5000 daltons, for example less than 4000, 3000, 2000 or 1000 daltons, or with a molecule weight less than 500 daltons, for example less than 450 daltons, 400 daltons, 350 daltons, 300 daltons, 250 daltons, 200 daltons, 150 daltons, 100 daltons, 50 daltons or 10 daltons.

It is further preferred if the binding partner causes a change (i.e a modulation) in the level of biological activity of the CRF1R, i.e. it has functional agonist or antagonist activity, and therefore may have the potential to be a candidate drug. Thus, the binding partner may be any of a full agonist, a partial agonist, an inverse agonist or an antagonist of CRF1R. The binding partner may bind to the orthosteric site or it may bind to an allosteric binding site. It is also appreciated that the binding partner may be one that modulates the ability of the CRF1R to dimerise. For example, the binding partner may bind to the dimerisation interface or bind to another region of the CRF1R which nevertheless modulates dimerisation.

Accordingly, a third aspect of the invention provides a method for selecting or designing one or more binding partners of CRF1R comprising using molecular modelling means to select or design one or more binding partners of the CRF1R, wherein the three-dimensional structural representation of at least part of the human CRF1R, as defined by coordinates of the CRF1R, optionally varied by a root mean square deviation of residue backbone atoms of not more than 4.383 Å, or selected coordinates thereof, is compared with a three-dimensional structural representation of one or more candidate binding partners, and one or more binding partners that are predicted to interact with CRF1R are selected.

In order to provide a three-dimensional structural representation of a candidate binding partner, the binding partner structural representation may be modelled in three dimensions using commercially available software for this purpose or, if its crystal structure is available, the coordinates of the structure may be used to provide a structural representation of the binding partner.

The design of binding partners that bind to a CRF1R generally involves consideration of two factors.

First, the binding partner must be capable of physically and structurally associating with parts or all of a CRF1R binding region (e.g. orthosteric binding site or an allosteric binding site). Non-covalent molecular interactions important in this association include hydrogen bonding, van der Waals interactions, hydrophobic interactions and electrostatic interactions.

Second, the binding partner must be able to assume a conformation that allows it to associate with a CRF1R binding region directly. Although certain portions of the binding partner will not directly participate in these associations, those portions of the binding partner may still influence the overall conformation of the molecule. This, in turn, may have a significant impact on potency. Such conformational requirements include the overall three-dimensional structure and orientation of the binding partner in relation to all or a portion of the binding region, or the spacing between functional groups of a binding partner comprising several binding partners that directly interact with the CRF1R. This is particularly relevant where the binding partner is a protein.

Thus it will be appreciated that selected coordinates which represent a binding region of the CRF1R, e.g. atoms from amino acid residues contributing to the small organic molecule binding pocket including amino acid residues Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362, may be used, or atoms from amino acid residues contributing to the peptide orthosteric binding site including amino acid residues Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355, may be used. Selected coordinates representing an extracellular face would be useful to select or design for binding partners such as antibodies, and selected coordinates representing an intracellular face would be useful to select or design for agents which modulate (e.g. prevent) binding to natural binding partners such as G proteins. Additional preferences for the selected coordinates are as defined above with respect to the first aspect of the invention. Preferably, the selected coordinates comprise one or more atoms from any one or more (eg at least 2, 3, 4, 5, 6 or 7) of amino acids Phe 203, Met 206, Gly 210, Asn 283, Thr 316, Leu 323 and Tyr 327, according to the numbering of the CRF1R sequence as set out in FIG. 13, which include the nearest residues to the ligand.

Designing of binding partners can generally be achieved in two ways, either by the step wise assembly of a binding partner or by the de novo synthesis of a binding partner. As is described in more detail below, binding partners can also be identified by virtual screening.

With respect to the step-wise assembly of a binding partner, several methods may be used. Typically the process begins by visual inspection of, for example, any of the binding regions on a computer representation of the CRF1R as defined by the coordinates in Table A or Table B or Table C optionally varied within a rmsd of residue backbone atoms of not more than 4.383 Å, or selected coordinates thereof. Selected binding partners, or fragments or moieties thereof may then be positioned in a variety of orientations, or docked, within the binding region. Docking may be accomplished using software such as QUANTA and Sybyl (Tripos Associates, St. Louis, Mo.), followed by, or performed simultaneously with, energy minimization, rigid-body minimization (Gshwend, supra) and molecular dynamics with standard molecular mechanics force fields, such as CHARMM and AMBER.

Specialized computer programs may also assist in the process of selecting binding partners or fragments or moieties thereof, as are known in the art and as detailed in WO2008/068534 incorporated herein by reference.

Once suitable binding partners or fragments have been selected, they may be assembled into a single compound or complex. Assembly may be preceded by visual inspection of the relationship of the fragments to each other on the three-dimensional image displayed on a computer screen in relation to the structure coordinates of the CRF1R. This would be followed by manual model building using software such as QUANTA or Sybyl. Useful programs known in the art (see, for example WO2008/068534 incorporated herein by reference) may aid connecting the individual chemical entities or fragments.

Thus the invention includes a method of designing a binding partner of a CRF1R comprising the steps of: (a) providing a structural representation of a CRF1R binding region as defined by the coordinates of the human CRF1R of Table A or Table B or Table C, optionally varied by a root mean square deviation of residue backbone atoms of not more than 4.383 Å or selected coordinates thereof; (b) using computational means to dock a three dimensional structural representation of a first binding partner in part of the binding region; (c) docking at least a second binding partner in another part of the binding region; (d) quantifying the interaction energy between the first or second binding partner and part of the binding region; (e) repeating steps (b) to (d) with another first and second binding partner, selecting a first and a second binding partner based on the quantified interaction energy of all of said first and second binding partners; (f) optionally, visually inspecting the relationship of the first and second binding partner to each other in relation to the binding region; and (g) assembling the first and second binding partners into a one binding partner that interacts with the binding region by model building.

As an alternative to the step-wise assembly of binding partners, binding partners may be designed as a whole or “de novo” using either an empty binding region or optionally including some portion(s) of a known binding partner(s). There are many de novo ligand design methods including: 1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.; 2. LEGEND (Y. Nishibata et al., Tetrahedron, 47, p. 8985 (1991)). LEGEND is available from Molecular Simulations Incorporated, San Diego, Calif.; 3. LeapFrog (available from Tripos Associates, St. Louis, Mo.); and 4. SPROUT (V. Gillet et al., “SPROUT: A Program for Structure Generation)”, J. Comput. Aided Mol. Design, 7, pp. 127-153 (1993)). SPROUT is available from the University of Leeds, UK.

Other molecular modelling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)).

In addition to the methods described above in relation to the design of binding partners, other computer-based methods are available to select for binding partners that interact with CRF1R.

For example the invention involves the computational screening of small molecule databases for binding partners that can bind in whole, or in part, to the CRF1R. In this screening, the quality of fit of such binding partners to a binding region of a CRF1R as defined by the coordinates of the human CRF1R of Table A or Table B or Table C, optionally varied by a root mean square deviation of residue backbone atoms of not more than 4.383 Å or selected coordinates thereof, may be judged either by shape complementarity or by estimated interaction energy (E. C. Meng et al., J. Comp. Chem., 13, pp. 505-524 (1992)).

For example, selection may involve using a computer for selecting an orientation of a binding partner with a favourable shape complementarity in a binding region comprising the steps of: (a) providing the coordinates of the human CRF1R of Table A or Table B or Table C, optionally varied by a root mean square deviation of residue backbone atoms of not more than 4.383 Å or selected coordinates thereof and a three-dimensional structural representation of one or more candidate binding partners; (b) employing computational means to dock a first binding partner in the binding region; (c) quantitating the contact score of the binding partner in different orientations; and (d) selecting an orientation with the highest contact score.

The docking may be facilitated by the contact score. The method may further comprise the step of generating a three-dimensional structural representation of the binding region and binding partner bound therein prior to step (b).

The method may further comprise the steps of: (e) repeating steps (b) through (d) with a second binding partner; and (f) selecting at least one of the first or second binding partner that has a higher contact score based on the quantitated contact score of the first or second binding partner.

In another embodiment, selection may involve using a computer for selecting an orientation of a binding partner that interacts favourably with a binding region comprising; a) providing the coordinates of the human CRF1R of Table A or Table B or Table C, optionally varied by a root mean square deviation of residue backbone atoms of not more than 4.383 Å or selected coordinates thereof; b) employing computational means to dock a first binding partner in the binding region; c) quantitating the interaction energy between the binding partner and all or part of a binding region for different orientations of the binding partner; and d) selecting the orientation of the binding partner with the most favorable interaction energy.

The docking may be facilitated by the quantitated interaction energy and energy minimization with or without molecular dynamics simulations may be performed simultaneously with or following step (b).

The method may further comprise the steps of: (e) repeating steps (b) through (d) with a second binding partner; and (f) selecting at least one of the first or second binding partner that interacts more favourably with a binding region based on the quantitated interaction energy of the first or second binding partner.

In another embodiment, selection may involve screening a binding partner to associate with an energy of binding of less than −7 kcal/mol with an CRF1R binding region comprising: (a) providing the coordinates of the human CRF1R of Table A or Table B or Table C, optionally varied by a root mean square deviation of residue backbone atoms of not more than 4.383 Å or selected coordinates thereof and employing computational means which utilise coordinates to dock the binding partner into a binding region; (b) quantifying the deformation energy of binding between the binding partner and the binding region; and (d) selecting a binding partner that associates with a CRF1R binding region with an energy of binding of less than −7 kcal/mol.

A fourth aspect of the invention provides a method for selecting or designing one or more binding partners of a CRF1R having a binding pocket in the position structurally equivalent to a binding pocket of human CRF1R that is defined by residues including (a) Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362 of human CRF1R or (b) Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355 of human CRF1R, the method comprising the step of using molecular modelling means to select or design one or more binding partners that are predicted to interact with the said CRF1R, wherein a three-dimensional structural representation of one or more candidate binding partners are compared with a three-dimensional structural representation of the said binding pocket, and one or more candidate binding partners that are predicted to interact with the said binding pocket, are selected or designed.

Preferably, the binding partner selected is one that is able to interact with at least one of amino acids that define the said binding pockets such as at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all 41 of said amino acid residues.

By a CRF1R having a binding pocket in the position structurally equivalent to the defined binding pocket of human CRF1R, we include the meaning of a protein identifiable as that of a CRF1R, and further having a predicted or determined three-dimensional structure that includes a binding pocket defined by (a) Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362 according to the numbering of the human CRF1R in FIG. 13 or (b) Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355 according to the numbering of the human CRF1R in FIG. 13. An amino acid sequence may be identifiable as that of a CRF1R by reference to sequence identity or similarities of three dimensional structure with known CRF1Rs, as known to those skilled in the art.

It will be appreciated that the three-dimensional structural representations of the defined binding pockets may be any suitable three-dimensional structural representation. For example, it may be a three-dimensional structural representation represented by the coordinates of the CRF1R structure in Table A or Table B or Table C, optionally varied by a root mean square deviation of residue backbone atoms of not more than 4.383 Å, or selected coordinates thereof. It is preferred if the selected coordinates are from one or more amino acid residues that define a binding region of CRF1R including those mentioned above. Alternatively, the three-dimensional structural representations of the defined binding pockets may be a three-dimensional structural representation modelled on such coordinates.

The structural representation may then be compared with structural representations of one or more candidate binding partners and those binding partners that are predicted to interact with the binding pocket are selected.

Any suitable molecular modelling means may be employed in this selection, including those outlined above.

It is appreciated that in some instances high throughput screening of binding partners is preferred and that methods of the invention may be used as “library screening” methods, a term well known to those skilled in the art. Thus, the binding partner may be a library of binding partners. For example, the library may be a peptide or protein library produced, for example, by ribosome display or an antibody library prepared either in vivo, ex vivo or in vitro. Methodologies for preparing and screening such libraries are known in the art.

Determination of the three-dimensional structure of the CRF1R provides important information about the binding sites of CRF1R receptors, particularly when comparisons are made with other GPCRs including corticotropin factor receptors. This information may then be used for rational design and modification of CRF1R binding partners, e.g. by computational techniques which identify possible binding ligands for the binding sites, by enabling linked-fragment approaches to drug design, and by enabling the identification and location of bound ligands using X-ray crystallographic analysis. These techniques are discussed in more detail below.

Thus as a result of the determination of the CRF1R three-dimensional structure, more purely computational techniques for rational drug design may also be used to design structures whose interaction with CRF1R is better understood (for an overview of these techniques see e.g. Walters et al (Drug Discovery Today, Vol. 3, No. 4, (1998), 160-178; Abagyan, R.; Totrov, M. Curr. Opin. Chem. Biol. 2001, 5, 375-382). For example, automated ligand-receptor docking programs (discussed e.g. by Jones et al. in Current Opinion in Biotechnology, Vol. 6, (1995), 652-656 and Halperin, I.; Ma, B.; Wolfson, H.; Nussinov, R. Proteins 2002, 47, 409-443), which require accurate information on the atomic coordinates of target receptors may be used.

The aspects of the invention described herein which utilize the CRF1R structure in silico may be equally applied to both the human CRF1R structure of Table A or Table B or Table C, optionally varied by a root mean square deviation of residue backbone atoms of not more than 4.383 Å, or selected coordinates thereof; and, by predicting the three-dimensional structural representation of a target protein, or part thereof, by modelling the structural representation on all or the selected coordinates of the CRF1R or selected coordinates thereof, to the models of target proteins obtained by the first and second aspects of the invention. Thus having determined a conformation of a target protein, for example an CRF1R, by the methods described above, such a conformation may be used in a computer-based method of rational drug design as described herein. In addition, the availability of the structure of the CRF1R will allow the generation of highly predictive pharmacophore models for virtual library screening or ligand design.

Accordingly, a fifth aspect of the invention provides a method for the analysis of the interaction of one or more binding partners with CRF1R, comprising: providing a three dimensional structural representation of CRF1R as defined by the coordinates of the human CRF1R structure of Table A or Table B or Table C, optionally varied by a root mean square deviation of residue backbone atoms of not more than 4.383 Å, or selected coordinates thereof; providing a three dimensional structural representation of one or more binding partners to be fitted to the structural representation of CRF1R or selected coordinates thereof; and fitting the one of more binding partners to said structure.

This method of the invention is generally applicable for the analysis of known binding partners of CRF1R, the development or discovery of binding partners of CRF1R, the modification of binding partners of CRF1R e.g. to improve or modify one or more of their properties, and the like. Moreover, the methods of the invention are useful in identifying binding partners that are selective for CRF1R receptors over other GPCRs (including other corticotropin factor receptors). For example, comparing corresponding binding regions between CRF1R receptors and other GPCRs will facilitate the design of CRF1R specific binding partners.

It will be desirable to model a sufficient number of atoms of the CRF1R as defined by the coordinates of Table A or Table B or Table C optionally varied by a root mean square deviation of residue backbone atoms of not more than 4.383 Å, or selected coordinates thereof, which represent a binding region, e.g. atoms from amino acid residues contributing to the small organic molecule binding pocket including amino acid residues Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362, or atoms from amino acid residues contributing to the peptide orthosteric binding site including amino acid residues Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355. Although every different binding partner bound by CRF1R may interact with different parts of a binding region of the protein, the structure of the CRF1R allows the identification of a number of particular sites which are likely to be involved in many of the interactions of CRF1R with a drug candidate. Additional preferred selected coordinates are as described as above with respect to the first aspect of the invention.

In order to provide a three-dimensional structural representation of a binding partner to be fitted to the CRF1R structure, the binding partner structural representation may be modelled in three dimensions using commercially available software for this purpose or, if its crystal structure is available, the coordinates of the structure may be used to provide a structural representation of the binding partner for fitting to the CRF1R structure of the invention.

By “fitting”, is meant determining by automatic, or semi-automatic means, interactions between one or more atoms of a candidate binding partner and at least one atom of the CRF1R structure of the invention, and calculating the extent to which such interactions are stable. Interactions include attraction and repulsion, brought about by charge, steric, lipophilic, considerations and the like. Charge and steric interactions of this type can be modelled computationally. An example of such computation would be via a force field such as Amber (Cornell et a/. A Second Generation Force Field for the Simulation of Proteins, Nucleic Acids, and Organic Molecules, Journal of the American Chemical Society, (1995), 117(19), 5179-97) which would assign partial charges to atoms on the protein and binding partner and evaluate the electrostatic interaction energy between a protein and binding partner atom using the Coulomb potential. The Amber force field would also assign van der Waals energy terms to assess the attractive and repulsive steric interactions between two atoms. Lipophilic interactions can be modeled using a variety of means. For example the ChemScore function (Eldridge M D; Murray C W; Auton T R; Paolini G V; Mee R P Empirical scoring functions: I. The development of a fast empirical scoring function to estimate the binding affinity of binding partners in receptor complexes, Journal of computer-aided molecular design (1997 September), 11 (5), 425-45) assigns protein and binding partner atoms as hydrophobic or polar, and a favourable energy term is specified for the interaction between two hydrophobic atoms. Other methods of assessing the hydrophobic contributions to ligand binding are available and these would be known to one skilled in the art. Other methods of assessing interactions are available and would be known to one skilled in the art of designing molecules. Various computer-based methods for fitting are described further herein.

More specifically, the interaction of a binding partner with the CRF1R structure of the invention can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, (1982) J. Mol. Biol., 161, 269-288; Makino et al, (1997) J. Comput. Chem., 18, 1812-1825), AUTODOCK (Goodsell et al, (1990) Proteins, 8, 195-202, Morris et al, (1998) J. Comput. Chem., 19, 1639-1662.), Glide (Friesner et al (2004) J. Med. Chem. 47, 1739-1749), FlexX, (Rarey et al, (1996) J. Mol. Biol., 261, 470-489) or ICM (Abagyan et al, (1994) J. Comput. Chem., 15, 488-506). This procedure can include computer fitting of binding partners to the CRF1R structure to ascertain how well the shape and the chemical structure of the binding partner will bind to a CRF1R.

Thus the invention includes a method for the analysis of the interaction of one or more binding partners with CRF1R comprising (a) constructing a computer representation of a binding region of the CRF1R as defined by the coordinates of the human CRF1R of Table A or Table B or Table C, optionally varied by a root mean square deviation of residue backbone atoms of not more than 4.383 Å or selected coordinates thereof (b) selecting a binding partner to be evaluated by a method selected from the group consisting of assembling said binding partner; selecting a binding partner from a small molecule database; de novo ligand design of the binding partner; and modifying a known agonist or inhibitor, or a portion thereof, of a CRF1R or homologue thereof; (c) employing computational means to dock said binding partner to be evaluated in a binding region in order to provide an energy-minimized configuration of the binding partner in a binding region; and (d) evaluating the results of said docking to quantify the interaction energy between said binding partner and the binding region.

Also computer-assisted, manual examination of the binding region structure of the CRF1R may be performed. The use of programs such as GRID (Goodford, (1985) J. Med. Chem., 28, 849-857)—a program that determines probable interaction sites between molecules with various functional groups and an enzyme surface—may also be used to analyse a binding region to predict, for example, the types of modifications which will alter the rate of metabolism of a binding partner.

Computer programs can be employed to estimate the attraction, repulsion, and steric hindrance of the CRF1R structure and a binding partner.

Further modelling software that may be used in the context of the invention include MOE (Molecular Operating Environment; Chemical Computing Group Inc., 1010 Sherbooke St. West, Suite #910, Montreal, QC, Canada, H3A 2R7), Maestro (Schrödinger, LLC, New York, N.Y., 2012), and Discovery Studio (Accelrys Software Inc., Discovery Studio Modeling Environment, Release 3.5, San Diego: Accelrys Software Inc., 2012).

If more than one CRF1R binding region is characterized and a plurality of respective smaller molecular fragments are designed or selected, a binding partner may be formed by linking the respective small molecular fragments into a single binding partner, which maintains the relative positions and orientations of the respective small molecular fragments at the binding sites. The single larger binding partner may be formed as a real molecule or by computer modelling. Detailed structural information can then be obtained about the binding of the binding partner to CRF1R, and in the light of this information adjustments can be made to the structure or functionality of the binding partner, e.g. to alter its interaction with CRF1R. The above steps may be repeated and re-repeated as necessary.

Thus, the three dimensional structural representation of the one or more binding partners of the third, fourth and fifth aspects of the invention may be obtained by: providing structural representations of a plurality of molecular fragments; fitting the structural representation of each of the molecular fragments to the coordinates of the human CRF1R structural representation of Table A or Table B or Table C, optionally varied by a root mean square deviation of residue backbone atoms of not more than 4.383 Å, or selected coordinates thereof; and assembling the representations of the molecular fragments into one or more representations of single molecules to provide the three-dimensional structural representation of one or more candidate binding partners.

Typically the binding partner or molecule fragment is fitted to at least 5 or 10 non-hydrogen atoms of the CRF1R structure, preferably at least 20, 30, 40, 50, 60, 70, 80 or 90 non-hydrogen atoms and more preferably at least 100, 150, 200, 250, 300, 350, 400, 450, or 500 atoms and even more preferably at least 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300 or 3400 non-hydrogen atoms.

The invention includes screening methods to identify drugs or lead compounds of use in treating a disease or condition. For example, large numbers of binding partners, for example in a chemical database, can be screened for their ability to bind to CRF1R.

It is appreciated that in the methods described herein, which may be drug screening methods, a term well known to those skilled in the art, the binding partner may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons (such as less than 500 daltons) and which may be water-soluble. A drug-like compound may additionally exhibit features of selective interaction with a particular protein or proteins and be bioavailable and/or able to penetrate target cellular membranes or the blood:brain barrier, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

Thus in one embodiment of the methods of third, fourth and fifth aspects of the invention, the methods further comprise modifying the structural representation of the binding partner so as to increase or decrease their interaction with CRF1R.

For example, once a binding partner has been designed or selected by the above methods, the efficiency with which that binding partner may bind to a CRF1R may be tested and optimised, for example by computational evaluation. For example, a binding partner designed or selected as binding to a CRF1R may be further computationally optimised so that in its bound state it would preferably lack repulsive electrostatic interaction with the target CRF1R and with the surrounding water molecules. Such non-complementary electrostatic interactions include repulsive charge-charge, dipole-dipole and charge-dipole interactions.

Furthermore, it is often desired that binding partners demonstrate a relatively small difference in energy between the bound and free states (i.e., a small deformation energy of binding). Thus, binding partners may be designed with a deformation energy of binding of not greater than about 10 kcal/mole, more preferably, not greater than 7 kcal/mole. Binding partners may interact with the binding region in more than one conformation that is similar in overall binding energy. In those cases, the deformation energy of binding is taken to be the difference between the energy of the free binding partner and the average energy of the conformations observed when the binding partner binds to the protein.

Specific computer software is available in the art to evaluate compound deformation energy and electrostatic interactions as detailed in WO2008/068534 (see, for example, page 34) incorporated herein by reference.

By modifying the structural representation we include, for example, adding molecular scaffolding, adding or varying functional groups, or connecting the molecule with other molecules (e.g. using a fragment linking approach) such that the chemical structure of the binding partner is changed while its original binding to CRF1R capability is increased or decreased. Such optimisation is regularly undertaken during drug development programmes to e.g. enhance potency, promote pharmacological acceptability, increase chemical stability etc. of lead compounds.

Examples of modifications include substitutions or removal of groups containing residues which interact with the amino acid side chain groups of the CRF1R structure of the invention, as described further in relation to the 6-adrenergic receptor in WO2008/068534 (see for example, page 35), incorporated herein by reference.

The potential binding effect of a binding partner on CRF1R may be analysed prior to its actual synthesis and testing by the use of computer modeling techniques. If the theoretical structure of the given entity suggests insufficient interaction and association between it and the CRF1R, testing of the entity is obviated. However, if computer modelling indicates a strong interaction, the molecule may then be synthesized and tested for its ability to bind to a CRF1R. In this manner, synthesis of inoperative compounds may be avoided.

Thus in a further embodiment of the third, fourth and fifth aspects of the invention, the methods further comprise the steps of obtaining or synthesising the one or more binding partners of a CRF1R; and optionally contacting the one or more binding partners with a CRF1R to determine the ability of the one or more binding partners to interact with the CRF1R.

Various methods known in the art may be used to determine binding between a CRF1R and a binding partner including those described in WO2008/068534 (see for example, pages 35-36) incorporated herein by reference.

Once computer modelling has indicated that a binding partner has a strong interaction, it is appreciated that it may be desirable to crystallise a complex of the CRF1R with that binding partner and analyse its interaction further by X-ray crystallography.

Thus in a further embodiment of the third, fourth and fifth aspects of the invention, the methods further comprise the steps of obtaining or synthesising the one or more binding partners of a CRF1R; forming one or more complexes of the CRF1R and the one or more binding partners; and analysing the one or more complexes by X-ray crystallography to determine the ability of the one or more binding partners to interact with CRF1R.

Thus, it will be appreciated that another particularly useful drug design technique enabled by this invention is iterative drug design. Iterative drug design is a method for optimizing associations between a protein and a binding partner by determining and evaluating the three-dimensional structures of successive sets of protein/compound complexes, and is described further in WO2008/068534 (see, for example, pages 36-37), incorporated herein by reference.

The ability of a binding partner to modify CRF1R function may also be tested. For example the ability of a binding partner to modulate a CRF1R function could be tested by a number of well known standard methods, described extensively in the prior art.

In addition to in silico analysis and design, the interaction of one or more binding partners with a CRF1R may be analysed directly by X-ray crystallography experiments, wherein the coordinates of the human CRF1R of Table A or Table B or Table C optionally varied by a root mean square deviation of residue backbone atoms of not more than 4.383 Å, or selected coordinates thereof, are used to analyse a crystal complex of the CRF1R receptor and binding partner. This can provide high resolution information of the interaction and can also provide insights into a mechanism by which a binding partner exerts an agonistic or antagonistic function.

Accordingly, a sixth aspect of the invention provides a method for the analysis of the interaction of one or more binding partners with CRF1R, comprising: obtaining or synthesising one or more binding partners; forming one or more crystallised complexes of a CRF1R and a binding partner; and analysing the one or more complexes by X-ray crystallography by employing the coordinates of the human CRF1R structure, of Table A or Table B or Table C optionally varied by a root mean square deviation of residue backbone atoms of not more than 4.383 Å, or selected coordinates thereof, to determine the ability of the one or more binding partners to interact with the CRF1R.

Preferences for the selected coordinates in this and all subsequent aspects of the invention are as defined above with respect to the first aspect of the invention.

The analysis of such structures may employ X-ray crystallographic diffraction data from the complex and the coordinates of the human CRF1R structure, of Table A or Table B or Table C optionally varied by a root mean square deviation of residue backbone atoms of not more than 4.383 Å, or selected coordinates thereof, to generate a difference Fourier electron density map of the complex. The difference Fourier electron density map may then be analysed.

In one embodiment, the one or more crystallised complexes are formed by soaking a crystal of CRF1R with the binding partner to form a complex. Alternatively, the complexes may be obtained by cocrystallising the CRF1R with the binding partner. For example a purified CRF1R protein sample is incubated over a period of time (usually >1 hr) with a potential binding partner and the complex can then be screened for crystallization conditions. Alternatively, protein crystals containing a first binding partner can be back-soaked to remove this binding partner by placing the crystals into a stabilising solution in which the binding partner is not present. The resultant crystals can then be transferred into a second solution containing a second binding partner and used to produce an X-ray diffraction pattern of CRF1R complexed with the second binding partner.

The complexes can be analysed using X-ray diffraction methods, e.g. according to the approach described by Greer et al., (J of Medicinal Chemistry, Vol. 37, (1994), 1035-1054), and difference Fourier electron density maps can be calculated based on X-ray diffraction patterns of soaked or co-crystallized CRF1R and the solved structure of uncomplexed CRF1R. This is described further in WO2008/068534 (see, for example, pages 38-39), incorporated herein by reference.

This information may thus be used to optimise known classes of CRF1R binding partners and to design and synthesize novel classes of CRF1R binding partners, particularly those which have agonistic or antagonistic properties, and to design drugs with modified CRF1R interactions.

In one approach, the structure of a binding partner bound to a CRF1R may be determined by experiment. This will provide a starting point in the analysis of the binding partner bound to CRF1R thus providing those of skill in the art with a detailed insight as to how that particular binding partner interacts with CRF1R and the mechanism by which it exerts any function effect.

Many of the techniques and approaches applied to structure-based drug design described above rely at some stage on X-ray analysis to identify the binding position of a binding partner in a ligand-protein complex. A common way of doing this is to perform X-ray crystallography on the complex, produce a difference Fourier electron density map, and associate a particular pattern of electron density with the binding partner. However, in order to produce the map (as explained e.g. by Blundell et al., in Protein Crystallography, Academic Press, New York, London and San Francisco, (1976)), it is necessary to know beforehand the protein three dimensional structure (or at least a set of structure factors for the protein crystal). Therefore, determination of the CRF1R structure also allows difference Fourier electron density maps of CRF1R-binding partner complexes to be produced, determination of the binding position of the binding partner and hence may greatly assist the process of rational drug design.

Accordingly, a seventh aspect of the invention provides a method of predicting the three dimensional structure of a binding partner of unknown structure, or part thereof, which binds to CRF1R, comprising: providing the coordinates of the human CRF1R structure, listed in Table A or Table B or Table C, optionally varied by a root mean square deviation of residue backbone atoms of not more than 4.383 Å, or selected coordinates thereof; providing an X-ray diffraction pattern of CRF1R complexed with the binding partner; and using said coordinates to predict at least part of the structure coordinates of the binding partner.

In one embodiment, the X-ray diffraction pattern is obtained from a crystal formed by soaking a crystal of CRF1R with the binding partner to form a complex. Alternatively, the X-ray diffraction pattern is obtained from a crystal formed by cocrystallising the CRF1R with the binding partner as described above. Alternatively, protein crystals containing a first binding partner can be back-soaked to remove this binding partner and the resultant crystals transferred into a second solution containing a second binding partner as described above.

A mixture of compounds may be soaked or co-crystallized with a CRF1R crystal, wherein only one or some of the compounds may be expected to bind to the CRF1R. The mixture of compounds may comprise a ligand known to bind to CRF1R. As well as the structure of the complex, the identity of the complexing compound(s) is/are then determined.

Preferably, the methods of the previous aspects of the invention are computer-based. For example, typically the methods of the previous aspects of the invention make use of the computer systems and computer-readable storage mediums of the tenth and eleventh aspects of the invention.

An eighth aspect of the invention provides a method for producing a binding partner of CRF1R comprising: identifying a binding partner according to the third, fourth, fifth, sixth or seventh aspects of the invention and synthesising the binding partner.

The binding partner may be synthesised using any suitable technique known in the art including, for example, the techniques of synthetic chemistry, organic chemistry and molecular biology.

It will be appreciated that it may be desirable to test the binding partner in an in vivo or in vitro biological system in order to determine its binding and/or activity and/or its effectiveness. For example, its binding to a CRF1R may be assessed using any suitable binding assay known in the art including the examples described above. Alternatively, is ability to modulate the CRF1R's ability to form dimers may be assessed.

Moreover, its effect on CRF1R function in an in vivo or in vitro assay may be tested. For example, the effect of the binding partner on the CRF1R signalling pathway may be determined. For example, the activity may be measured by using a reporter polynucleotide to measure the activity of the CRF1R signalling pathway. By a reporter polynucleotide we include genes which encode a reporter protein whose activity may easily be assayed, for example β-galactosidase, chloramphenicol acetyl transferase (CAT) gene, luciferase or Green Fluorescent Protein (see, for example, Tan et al, 1996 EMBO J 15(17): 4629-42). Several techniques are available in the art to detect and measure expression of a reporter polynucleotide which would be suitable for use in the present invention. Many of these are available in kits both for determining expression in vitro and in vivo. Alternatively, signalling may be assayed by the analysis of downstream targets. For example, a particular protein whose expression is known to be under the control of a specific signalling pathway may be quantified. Protein levels in biological samples can be determined using any suitable method known in the art. For example, protein concentration can be studied by a range of antibody based methods including immunoassays, such as ELISAs, western blotting and radioimmunoassays.

A ninth aspect of the invention provides a binding partner produced by the method of the eighth aspect of the invention.

Following identification of a binding partner, it may be manufactured and/or used in the preparation, i.e. manufacture or formulation, of a composition such as a medicament, pharmaceutical composition or drug. These may be administered to individuals.

Accordingly, the invention includes a method for producing a medicament, pharmaceutical composition or drug, the process comprising: (a) providing a binding partner according to the eighth aspect of the invention and (b) preparing a medicament, pharmaceutical composition or drug containing the binding partner.

The medicaments may be used to treat any disorder or condition ameliorated by modulation of the CRF1R. Examples include anxiety, depression, schizophrenia, stress related disorders, post-operative ileus, Alzheimer's disease, insomnia, eating disorders such as anorexia, panic disorder, cardiovascular disease including heart failure, kidney disease, Cusing's Disease, disease of the immune system including psoriasis, asthma, rheumatoid arthritis, inflammatory bowel disease, stroke and migraine.

The invention also provides systems, particularly a computer system, intended to generate structures and/or perform optimisation of binding partner which interact with CRF1R, CRF1R homologues or analogues, complexes of CRF1R with binding partners, or complexes of CRF1R homologues or analogues with binding partners.

Accordingly, a tenth aspect of the invention provides a computer system, intended to generate three dimensional structural representations of CRF1R, CRF1R homologues or analogues, complexes of CRF1R with binding partners, or complexes of CRF1R homologues or analogues with binding partners, or, to analyse or optimise binding of binding partners to said CRF1R or homologues or analogues, or complexes thereof, the system containing computer-readable data comprising one or more of:
(a) the coordinates of the human CRF1R structure, listed in Table A or Table B or Table C, optionally varied by a root mean square deviation of residue backbone atoms of not more than 4.383 Å, or selected coordinates thereof;
(b) the coordinates of a target CRF1R homologue or analogue generated by homology modelling of the target based on the data in (a);
(c) the coordinates of a binding partner generated by interpreting X-ray crystallographic data or NMR data by reference to the coordinates of the human CRF1R structure, listed in Table A or Table B or Table C, optionally varied by a root mean square deviation of residue backbone atoms of not more than 4.383 Å, and
(d) structure factor data derivable from the coordinates of (a), (b) or (c).

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design. The computer system may further comprise a display coupled to the central-processing unit for displaying structural representations.

The invention also provides such systems containing atomic coordinate data of target proteins of unknown structure wherein such data has been generated according to the methods of the invention described herein based on the starting data provided in Table A or Table B or Table C optionally varied by a root mean square deviation of residue backbone atoms of not more than 4.383 Å, or selected coordinates thereof.

Such data is useful for a number of purposes, including the generation of structures to analyse the mechanisms of action of binding partners and/or to perform rational drug design of binding partners which interact with CRF1R, such as compounds which are agonists or antagonists.

An eleventh aspect of the invention provides a computer-readable storage medium, comprising a data storage material encoded with computer readable data, wherein the data comprises one or more of:
(a) the coordinates of the human CRF1R structure, listed in Table A or Table B or Table C, optionally varied by a root mean square deviation of residue backbone atoms of not more than 4.383 Å, or selected coordinates thereof;
(b) the coordinates of a target CRF1R receptor homologue or analogue generated by homology modelling of the target based on the data in (a);
(c) the coordinates of a binding partner generated by interpreting X-ray crystallographic data or NMR data by reference to the coordinates of the human CRF1R structure, listed in Table A or Table B or Table C, optionally varied by a root mean square deviation of residue backbone atoms of not more than 4.383 Å, or selected coordinates thereof, and
(d) structure factor data derivable from the coordinates of (a), (b) or (c).

The invention also includes a computer-readable storage medium comprising a data storage material encoded with a first set of computer-readable data comprising a Fourier transform of at least a portion of the structural coordinates of human CRF1R listed in Table A or Table B or Table C, optionally varied by a root mean square deviation of residue backbone atoms of not more than 4.383 Å, or selected coordinates thereof; which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

It will be appreciated the that the computer-readable storage media of the invention may comprise a data storage material encoded with any of the data generated by carrying out any of the methods of the invention relating to structure solution and selection/design of binding partners to CRF1R and drug design.

The invention also includes a method of preparing the computer-readable storage media of the invention comprising encoding a data storage material with the computer-readable data.

As used herein, “computer readable media” refers to any medium or media, which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the atomic coordinate data of the invention can be routinely accessed to model CRF1R or selected coordinates thereof. For example, RASMOL (Sayle et al., TIBS, Vol. 20, (1995), 374) is a publicly available computer software package, which allows access and analysis of atomic coordinate data for structure determination and/or rational drug design.

As used herein, “a computer system” refers to the hardware means, software means and data storage means used to analyse the atomic coordinate data of the invention. The minimum hardware means of the computer-based systems of the present invention comprises a central processing unit (CPU), input means, output means and data storage means. Desirably, a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows XP or IBM OS/2 operating systems. Apple and Linux based systems may be used.

A twelfth aspect of the invention provides a method for providing data for generating three dimensional structural representations of CRF1R, CRF1R homologues or analogues, complexes of CRF1R with binding partners, or complexes of CRF1R homologues or analogues with binding partners, or, for analysing or optimising binding of binding partners to said CRF1R or homologues or analogues, or complexes thereof, the method comprising:
(i) establishing communication with a remote device containing computer-readable data comprising at least one of:
   (a) the coordinates of the human CRF1R structure of Table A or Table B or Table C, optionally varied by a root mean square deviation of residue backbone atoms of not more than 4.383 Å, or selected coordinates thereof;
   (b) the coordinates of a target CRF1R homologue or analogue generated by homology modelling of the target based on the data in (a);
   (c) the coordinates of a binding partner generated by interpreting X-ray crystallographic data or NMR data by reference to the coordinates of the human CRF1R structure of Table A or Table B or Table C, optionally varied by a root mean square deviation of residue backbone atoms of not more than 4.383 Å, or selected coordinates thereof, and
   (d) structure factor data derivable from the coordinates of (a), (b) or (c); and
(ii) receiving said computer-readable data from said remote device.

The computer-readable data received from said remote device, particularly when in the form of the coordinates of the human CRF1R structure of Table A or Table B or Table C, optionally varied by a root mean square deviation of residue backbone atoms of not more than 4.383 Å, or selected coordinates thereof, may be used in the methods of the invention described herein, e.g. for the analysis of a binding partner structure with a CRF1R structure.

Thus the remote device may comprise e.g. a computer system or computer readable media of one of the previous aspects of the invention. The device may be in a different country or jurisdiction from where the computer-readable data is received.

The communication may be via the internet, intranet, e-mail etc, transmitted through wires or by wireless means such as by terrestrial radio or by satellite. Typically the communication will be electronic in nature, but some or all of the communication pathway may be optical, for example, over optical fibers.

A thirteenth aspect of the invention provides a method of obtaining a three dimensional structural representation of a crystal of a CRF1R, which method comprises providing the coordinates of the human CRF1R structure of Table A or Table B or Table C, optionally varied by a root mean square deviation of residue backbone atoms of not more than 4.383 Å, or selected coordinates thereof, and generating a three-dimensional structural representation of said coordinates.

For example, the structural representation may be a physical representation or a computer generated representation. Examples of representations are described above and include, for example, any of a wire-frame model, a chicken-wire model, a ball-and-stick model, a space-filling model, a stick model, a ribbon model, a snake model, an arrow and cylinder model, an electron density map or a molecular surface model.

Computer representations can be generated or displayed by commercially available software programs including for example QUANTA (Accelrys .COPYRIGHT.2001, 2002), O (Jones et al., Acta Crystallogr. A47, pp. 110-119 (1991)), RIBBONS (Carson, J. Appl. Crystallogr., 24, pp. 9589-961 (1991)) and PyMol (The PyMOL Molecular Graphics System, Schrödinger LLC).

Typically, the computer used to generate the representation comprises (i) a computer-readable data storage medium comprising a data storage material encoded with computer-readable data, wherein said data comprise the coordinates of the human CRF1R structure of Table A or Table B or Table C, optionally varied by a root mean square deviation of residue backbone atoms of not more than 4.383 Å, or selected coordinates thereof; and (ii) instructions for processing the computer-readable data into a three-dimensional structural representation. The computer may further comprise a display for displaying said three-dimensional representation.

A fourteenth aspect of the invention provides a method of predicting one or more sites of interaction of a CRF1R or a homologue thereof, the method comprising: providing the coordinates of the human CRF1R structure of Table A or Table B or Table C, optionally varied by a root mean square deviation of residue backbone atoms of not more than 4.383 Å, or selected coordinates thereof; and analysing said coordinates to predict one or more sites of interaction.

For example, a binding region of a CRF1R for a particular binding partner can be predicted by modelling where the structure of the binding partner is known. Typically, the fitting and docking methods described above would be used. This method may be used, for example, to predict the site of interaction of a G protein of known structure as described in viz Gray J J (2006) Curr Op Struc Biol Vol 16, pp 183-193.

A fifteenth aspect of the invention provides a method for assessing the activation state of a structure for CRF1R, comprising: providing the the coordinates of the human CRF1R structure of Table A or Table B or Table C, optionally varied by a root mean square deviation of residue backbone atoms of not more than 4.383 Å, or selected coordinates thereof; performing a statistical and/or topological analysis of the coordinates; and comparing the results of the analysis with the results of an analysis of coordinates of proteins of known activation states.

For example, protein structures may be compared for similarity by statistical and/or topological analyses (suitable analyses are known in the art and include, for example those described in Grindley et al (1993) J Mol Biol Vol 229: 707-721 and Holm & Sander (1997) Nucl Acids Res Vol 25: 231-234). Highly similar scores would indicate a shared conformational and therefore functional state eg the inactive antagonist state in this case.

One example of statistical analysis is multivariate analysis which is well known in the art and can be done using techniques including principal components analysis, hierarchical cluster analysis, genetic algorithms and neural networks.

By performing a multivariate analysis of the coordinate data of the human CRF1R structure of Table A or Table B or Table C, optionally varied by a root mean square deviation of residue backbone atoms of not more than 4.383 Å, or selected coordinates thereof, and comparing the result of the analysis with the results of the analysis performed on coordinates of proteins with known activation states, it is possible to determine the activation state of the coordinate set analysed. For example, the activation state may be classified as ‘active’ or ‘inactive’.

A sixteenth aspect of the invention provides a method of producing a protein with a binding region that has substrate specificity substantially identical to that of CRF1R, the method comprising
a) aligning the amino acid sequence of a target protein with the amino acid sequence of a CRF1R;

b) identifying the amino acid residues in the target protein that correspond to any one or more of the following positions according to the numbering of the CRF1R as set out in FIG. 13: (i) Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362, or (ii) Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355; and
c) making one or more mutations in the amino acid sequence of the target protein to replace one or more identified amino acid residues with the corresponding residue in the CRF1R.

By “an amino acid residue that corresponds to” we include an amino acid residue that aligns to the given amino acid residue in CRF1R when the CRF1R and target protein are aligned using e.g. MacVector and CLUSTALW.

For example, amino acid residues contributing to the small organic molecule binding pocket of CRF1R include amino acid residues Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362, and amino acid residues contributing to the peptide orthosteric binding site include Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355. Thus a binding site of a particular protein may be engineered using well known molecular biology techniques to contain any one or more of these residues to give it the same substrate specificity. This technique is well known in the art and is described in, for example, Ikuta et al (J Biol Chem (2001) 276, 27548-27554) where the authors modified the active site of cdk2, for which they could obtain structural data, to resemble that of cdk4, for which no X-ray structure was available.

In the context of the small organic molecule binding site, preferably, all 41 amino acids in the target portion which correspond to amino acid residues Leu 158, Phe 162, His 199, Asn 202, Phe 203, Phe 204, Trp 205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362 of the CRF1R are, if different, replaced. However, it will be appreciated that only 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues may be replaced.

In the context of the peptide orthosteric binding site, preferably, all 41 amino acids in the target portion which correspond to amino acid residues Ala 119, Asn 123, His 127, Ser 130, Phe 162, Arg 165, Asn 166, Thr 168, Thr 169, Val 172, Gln 173, Thr 175, Met 176, His 181, Val 191, Thr 192, Tyr 195, Asn 196, His 199, Asn 202, Phe 203, Lys 257, Ala 260, Lys 262, Tyr 272, Gln 273, Met 276, Leu 323, Thr 326, Tyr 327, Ala 330, Phe 331, Asn 333, Asp 337, Arg 341, Phe 344, Ile 345, Asn 348, Glu 352, Ser 353 and Gln 355 of the CRF1R are, if different, replaced. However, it will be appreciated that only 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues may be replaced.

Preferences for the target protein are as defined above with respect to the first aspect of the invention.

A seventeenth aspect of the invention provides a method of predicting the location of internal and/or external parts of the structure of CRF1R or a homologue thereof, the method comprising: providing the coordinates of the CRF1R structure of Table A or Table B or Table C, optionally varied by a root mean square deviation of residue backbone atoms of not more than 4.383 Å, or selected coordinates thereof and analysing said coordinates to predict the location of internal and/or external parts of the structure.

For example, from the three dimensional representation, it is possible to read off external parts of the structure, eg surface residues, as well as internal parts, eg residues within the protein core. It will be appreciated that the identification of external protein sequences will be especially useful in the generation of antibodies against a CRF1R.

The crystallisation of the CRF1R has led to many interesting observations about its structure. Thus it will be appreciated that the invention allows for the generation of mutant CRF1Rs wherein residues corresponding to these areas of interest are mutated.

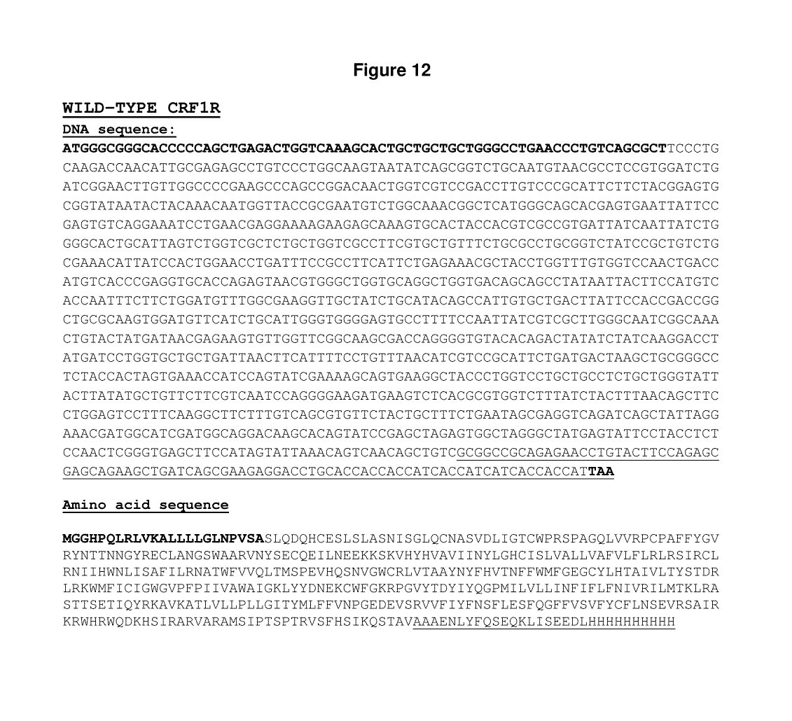

Accordingly, an eighteenth aspect of the invention provides a mutant CRF1R which, when compared to the corresponding wild-type CRF1R, has a different amino acid at a position which corresponds to any one or more of the following positions according to the numbering of the human CRF1R as set out in FIG. 12: Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362. As mentioned above, these amino acids contribute to a small organic molecule binding site in human CRF1R.

The invention also provides a mutant CRF1R which, when compared to the corresponding wild-type CRF1R, has a different amino acid at a position which corresponds to any one or more of the following positions according to the numbering of the human CRF1R as set out in FIG. 12: Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355. As mentioned above, these amino acids contribute to a peptide orthosteric binding site in human CRF1R.

A nineteenth aspect of the invention provides a mutant CRF1R which, when compared to the corresponding wild-type CRF1R has a different amino acid at a position which corresponds to any one or more of the following positions according to the numbering of the human CRF1R as set out in FIG. 12: Val 120, Leu 144, Trp 156, Ser 160, Ser 222, Lys 228, Phe 260, Ile 277, Tyr 309, Phe 330, Ser 349 and Tyr 363.

The inventors have found that these mutations increase the conformational stability of the GPCR (ie increase the stability of the mutant GPCR in a particular conformation compared to the stability of the parent GPCR in the same particular conformation), and so the mutant GPCR of the nineteenth aspect of the invention may be one which has increased conformational stability to any denaturant or denaturing condition such as to any one or more of heat, a detergent, a chaotropic agent or an extreme of pH. Suitable methods for assessing conformational stability are well known in the art and are described, for example, in WO 2008/114020. Conveniently, conformational stability is measured by an extended lifetime of the mutant under the imposed conditions which may lead to instability (such as heat, harsh detergent conditions, chaotropic agents and so on). Destabilisation under the imposed condition is typically determined by measuring denaturation or loss of structure. This may manifest itself by loss of ligand binding ability or loss of secondary or tertiary structure indicators.

Preferably, the mutant GPCR of the nineteenth aspect of the invention has increased stability in an agonist or antagonist conformation.

It is particularly preferred if the mutant CRF1R of the eighteenth or nineteenth aspects of the invention is one which has at least 20% amino acid sequence identity when compared to the given human CRF1R, as determined using MacVector and CLUSTALW. Preferably, the mutant CRF1R receptor has at least 30%, 40%, 50%, 60%, 70%, 80%, 85%, 90%, 95% or 99% amino acid sequence identity.

The mutant CRF1R receptor may be a mutant of any CRF1R receptor provided that it is mutated at one or more of the amino acid positions as stated by reference to the given human CRF1R amino acid sequence.

Thus, the invention includes a mutant human CRF1R in which, compared to its parent, one or more of these amino acid residues have been replaced by another amino acid residue. The invention also includes mutant CRF1Rs from other sources in which one or more corresponding amino acids in the parent receptor are replaced by another amino acid residue. For the avoidance of doubt the parent may be a CRF1R which has a naturally-occurring sequence, or it may be a truncated form or it may be a fusion, either to the naturally-occurring protein or to a fragment thereof, or it may contain mutations compared to the naturally-occurring sequence, providing that it retains its natural ligand-binding ability, ie it retains binding to CRF1.

For the avoidance of doubt, the mutant CRF1R of the invention, as described in the eighteenth and nineteenth aspects, is not a CRF1R with a naturally-occurring amino acid sequence.

In an embodiment of the eighteenth aspect, the mutant CRF1R of the invention has a combination of 2 or 3- or 4- or 5- or 6- or 7- or 8- or 9- or 10- or 11 or 12 or 13 or 14 or 15 or 16 or 17 or 18 or 19 or 20 or 21 or 22 or 23 or 24 or 25 or 26 or 27 or 28 or 29 or 30 or 31 or 32 or 33 or 34 of 35 or 36 or 37 or 38 or 39 or 40 or 41 mutations as described above.

In an embodiment of the nineteenth aspect, the mutant CRF1R of the invention has a combination of 2 or 3 or 4 or 5 or 6 or 7 or 8 or 9 or 10 or 11 or 12 mutations as described above.

It will be appreciated that it may be desirable to replace the intracellular loop (ICL)-2 of the mutant GPCR of the invention (eg of the nineteenth aspect of the invention) with T4 lysozyme so as to make the mutant CRF1R more amenable to crystallisation (see Example 1). By doing so, it may be desirable to remove the mutation at the position corresponding to Ser 222 according to the numbering of the human CRF1R as set out in FIG. 12. Thus, a particularly preferred mutant CRF1R of the invention is one which, when compared to the corresponding wild-type CRF1R, has a different amino acid at a position which corresponds to any one or more of (preferably all of) the following positions according to the numbering of the human CRF1R as set out in FIG. 12: Val 120, Leu 144, Trp 156, Ser 160, Lys 228, Phe 260, Ile 277, Tyr 309, Phe 330, Ser 349 and Tyr 363, and which mutant CRF1R comprises T4 lysozyme between TM3 and TM4 (eg ICL2 may be replaced with T4 lysozyme). Optionally, to aid crystallisation, one or both of the N- and C-termini of the mutant GPCR of the invention (eg of the nineteenth aspect of the invention) may be truncated (eg to remove the extracellular domain (ECD)). Thus, it will be appreciated that the invention provides a mutant CRF1R in which ICL2 is replaced with T4 lysozyme and in which one or both of the N- and C-termini are truncated (eg to remove the ECD).

By “corresponding amino acid residue” we include the meaning of the amino acid residue in another CRF1R receptor which aligns to the given amino acid residue in the human CRF1R when the human CRF1R and the other CRF1R are compared using MacVector and CLUSTALW.

Residues in proteins can be mutated using standard molecular biology techniques as are well known in the art.

Although the amino acid used to replace a given amino acid at a particular position is typically a naturally occurring amino acid, typically an “encodeable” amino acid, it may be a non-natural amino acid (in which case the protein is typically made by chemical synthesis or by use of non-natural amino-acyl tRNAs). An “encodeable” amino acid is one which is incorporated into a polypeptide by translation of mRNA. It is also possible to create non-natural amino acids or introduce non-peptide linkages at a given position by covalent chemical modification, for example by post-translational treatment of the protein or semisynthesis. These post-translational modifications may be natural, such as phosphorylation, glycosylation or palmitoylation, or synthetic or biosynthetic.

A twentieth aspect of the invention provides a method of making a CRF1R crystal comprising: providing purified CRF1R; and crystallising the CRF1R by using a lipidic cubic phase technique, using a precipitant solution comprising sodium citrate, lithium sulphate, and PEG. Preferably, the sodium citrate buffer has a concentration of between 20 and 200 mM such as 100 mM, and a pH of 4.5-6.5 such as a pH of 5.5. Any suitable PEG may be used. Generally, low molecular weight PEGs are used such as PEG200, PEG300, PEG400, PEG550mme, PEG600 and PEG1000. However, it is preferred if PEG400 is used.

In a particularly preferred embodiment, the precipitant solution comprises 100 mM sodium citrate pH 5.5, 200 mM lithium sulphate, and 30% (v/v) PEG400.

In a preferred embodiment, the a CRF1R ligand is included during the crystallisation process, for example CP-376395.

Preferably, the crystals are grown in lipidic cubic phase using a monoolein/cholesterol mixture, for example as described further in Example 1.

Accordingly, it will be appreciated that the precipitant solution may comprise 100 mM sodium citrate pH 5.5, 200 mM lithium sulphate, and 30% (v/v) PEG400; a CRF1R ligand may be included during the crystallisation process, for example CP-376395; and the crystals may be grown in lipidic cubic phase using a monoolein/cholesterol mixture.

A twenty-first aspect of the invention provides a crystal of CRF1R having the structure defined by the coordinates of the human CRF1R structure, listed in Table A or Table B or Table C, optionally varied by a root mean square deviation of residue backbone atoms of not more than 4.383 Å, or selected coordinates thereof. Typically, the crystal has a resolution of 3.15 Å or better, such as 2.97 Å or better.

The space group of the crystal may be P22₁2₁.

Thus, in one embodiment, the crystal has P22₁2₁ symmetry and unit cell dimensions a=86.6 (±15) Å, b=124.0 (±15) Å, c=166.8 (±15) Å. It will be appreciated that with P22₁2₁ symmetry all α, β and γ angles are 90°.

The invention also includes a co-crystal of CRF1A having the structure defined by the coordinates of the human CRF1R structure, listed in Table A or Table B or Table C, optionally varied by a root mean square deviation of residue backbone atoms of not more than 4.383 Å, or selected coordinates thereof, and a binding partner. Typically, the crystal has a resolution of 3.15 Å or better, such as 2.97 Å or better. The binding partner may be CP-376395.

In an embodiment of the twentieth and twenty-first aspects of the invention, the CRF1R is one in which intracellular loop (ICL) 2 is replaced with T4-lysozyme (T4L). Methods for inserting T4L into an ICL of a GPCR are routine practice in the art, and are described for example in Bill et al (Nat Biotechnol 29(4) 335-340 (2011)) and Kobilka et al (Science 240(4857) 1310-6 (1988)).

The invention includes the use of the coordinates of the CRF1R structure of Table A or Table B or Table C, optionally varied by a root mean square deviation of residue backbone atoms of not more than 4.383 Å, or selected coordinates thereof to solve the structure of target proteins of unknown structure.

The invention includes the use of the coordinates of the CRF1R structure of Table A or Table B or Table C, optionally varied by a root mean square deviation of residue backbone atoms of not more than 4.383 Å, or selected coordinates thereof to identify binding partners of an CRF1R.

The invention includes the use of the coordinates of the CRF1R structure of Table A or Table B or Table C, optionally varied by a root mean square deviation of residue backbone atoms of not more than 4.383 Å, or selected coordinates thereof in methods of drug design where the drugs are aimed at modifying the activity of the CRF1R.

The invention will now be described in more detail with the aid of the following Figures and Examples.

The invention will now be described with the aid of the following Figures and Examples.

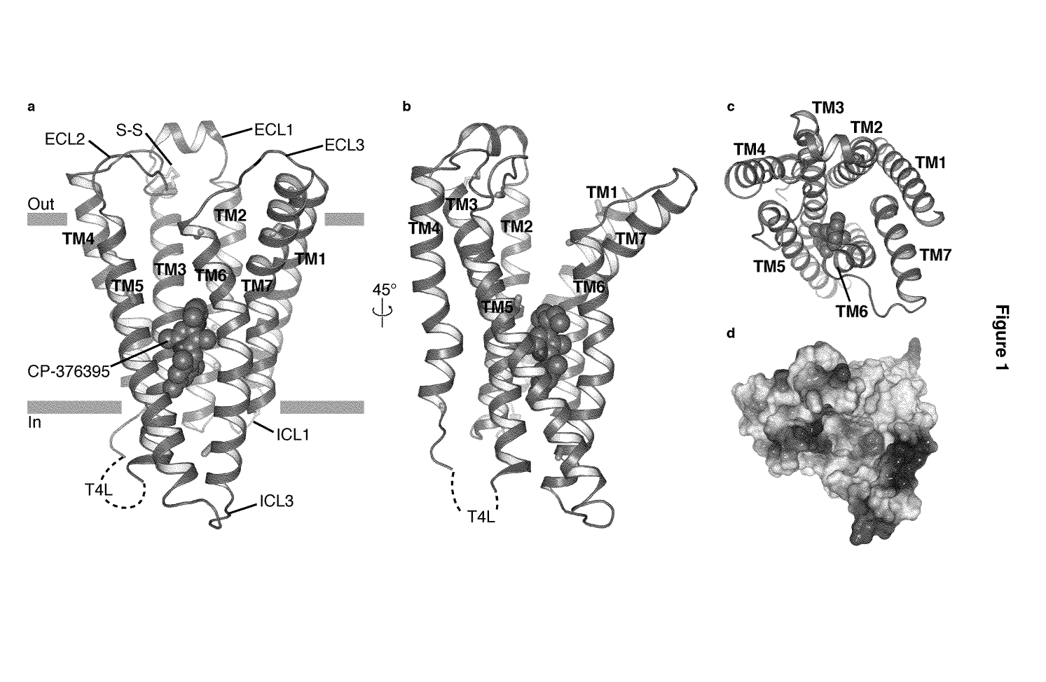

FIG. 1. Overall structure. In a and b, CRF₁R is shown in ribbons viewed from within the membrane from two angles. The antagonist CP-376395 is depicted in space fill representation. The disulfide bond linking ECL1 and TM3 is shown as sticks and labelled with S-S. The thermostabilizing mutations are rendered as sticks. The position of the T4L-insertion is indicated. In c, the receptor is viewed from the extracellular side. d, Same view as in c, but in a surface representation colored by electrostatic potential.

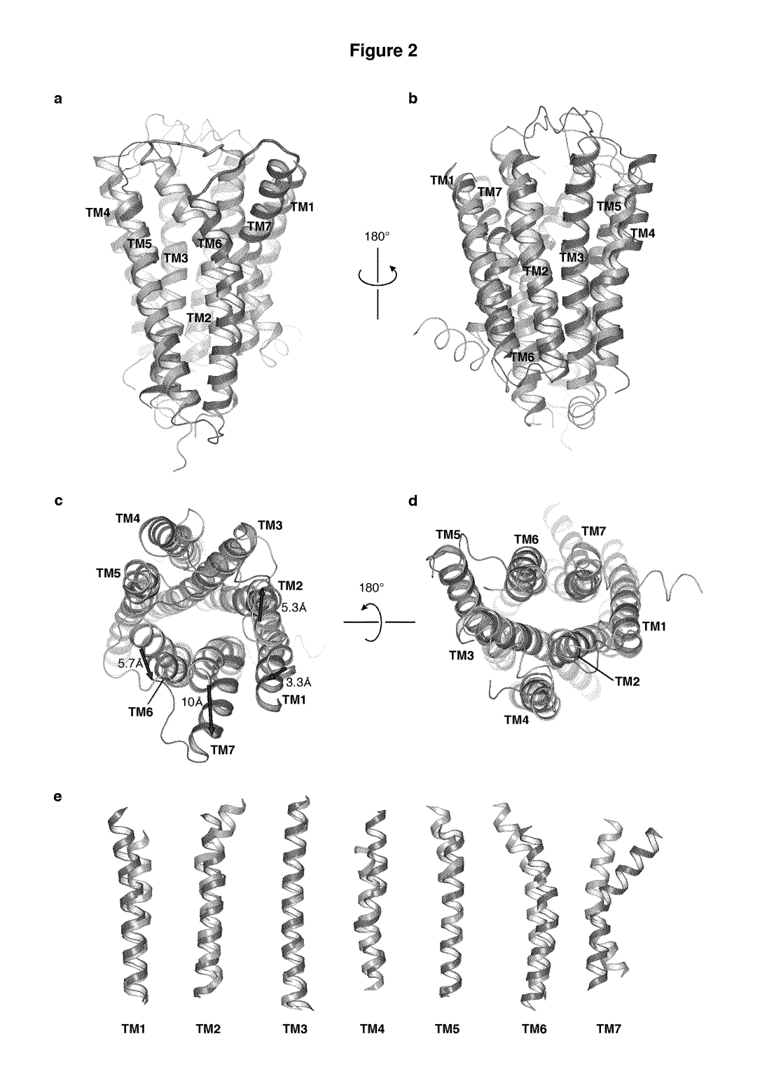

FIG. 2. Comparison of the antagonist-bound structures of CRF₁R and the dopamine D₃ receptor. The superimposed structures of CRF₁R and D₃R (PDB ID 3PBL) are shown as dark grey and light grey ribbons, respectively. The receptors are viewed from two different angles from within the membrane (a, b), from the extracellular side (c), and intracellular side (d). Arrows in c highlight large differences in the helical trajectories of the two receptors. In e, individual TM helices are shown after superposition of the two receptors as in a-d. The superposition of D₃R onto CRF₁R was done as described in Methods.

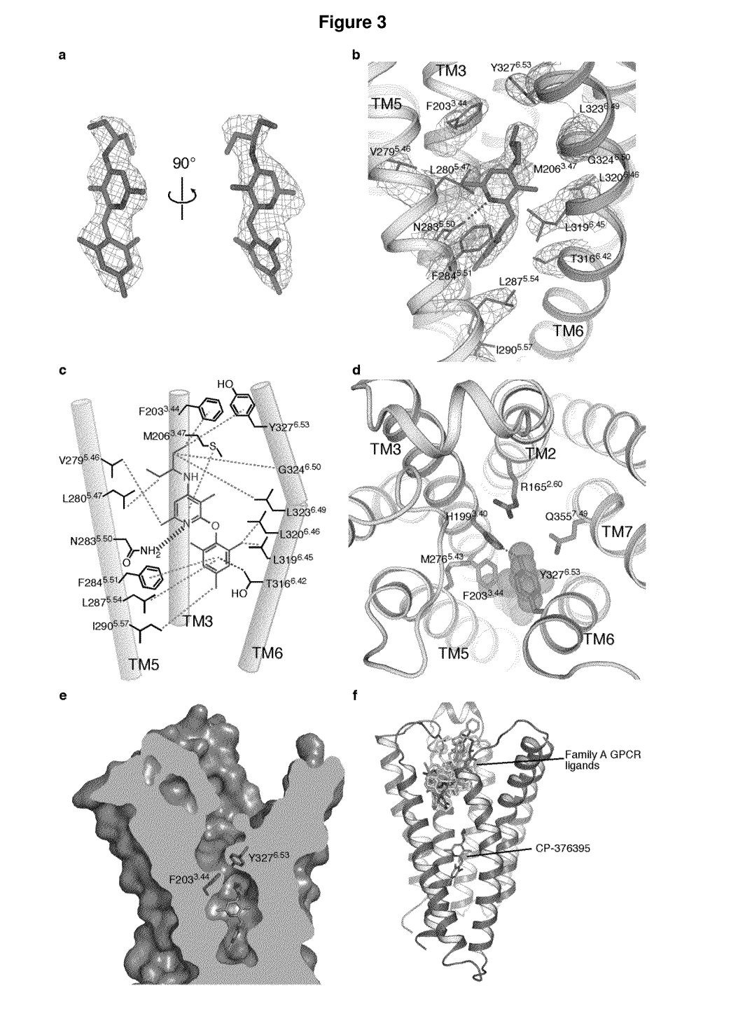

FIG. 3. Antagonist-binding site. a, The ligand is shown in Fₒ-F_{c} omit electron density within a 2 Å radius contoured at 3σ. b, The binding pocket viewed from within the membrane. 2Fₒ-F_{c} electron density within 2 Å of the binding site residues contoured at 1.5σ and the omit density is shown as in a. c, Schematic of the binding site. d, View from extracellular side onto the bottom of the putative orthosteric site. e, Cross-section of the solvent-accessible surface. F203^{3.44} and Y327^{6.53} are shown as sticks. f, The location of CP-376395 is compared to that of selected Class A receptor ligands.

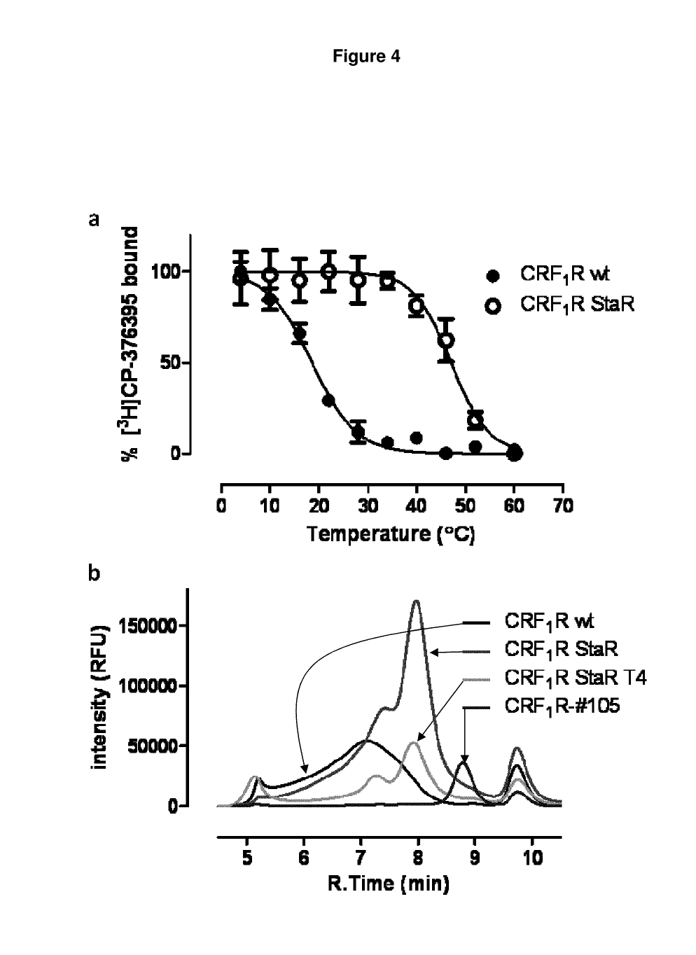

FIG. 4. Comparison of wild-type and stabilized CRF₁R. Panel a compares the thermal stability of wild-type CRF₁R (closed circles) to CRF₁R StaR (open circles) solubilised in 1% DDM, and measured using [³H]CP-376395 binding. Error bars are derived from standard deviation and calculated from duplicate temperature points (n=2) within a single experiment. See also Table 1. FSEC profiles of eGFP-tagged CRF₁R constructs are shown in panel b. Wild-type CRF₁R, CRF₁R StaR, CRF₁R StaR with T4 Lysozyme fusion and CRF₁R-#105 transiently transfected in HEK293T cells were solubilised in 1% DDM in the absence of ligand and analysed in running buffer containing 50 mM Tris-HCl pH 7.5, 150 mM NaCl and 0.03% DDM. The void volume elutes after 5 min, and free eGFP elutes after ˜9.5 min. Monomeric CRF₁R elutes at ˜8 min for full-length constructs and the truncated construct CRF₁R-#105 elutes as a monomeric species at ˜9 min.

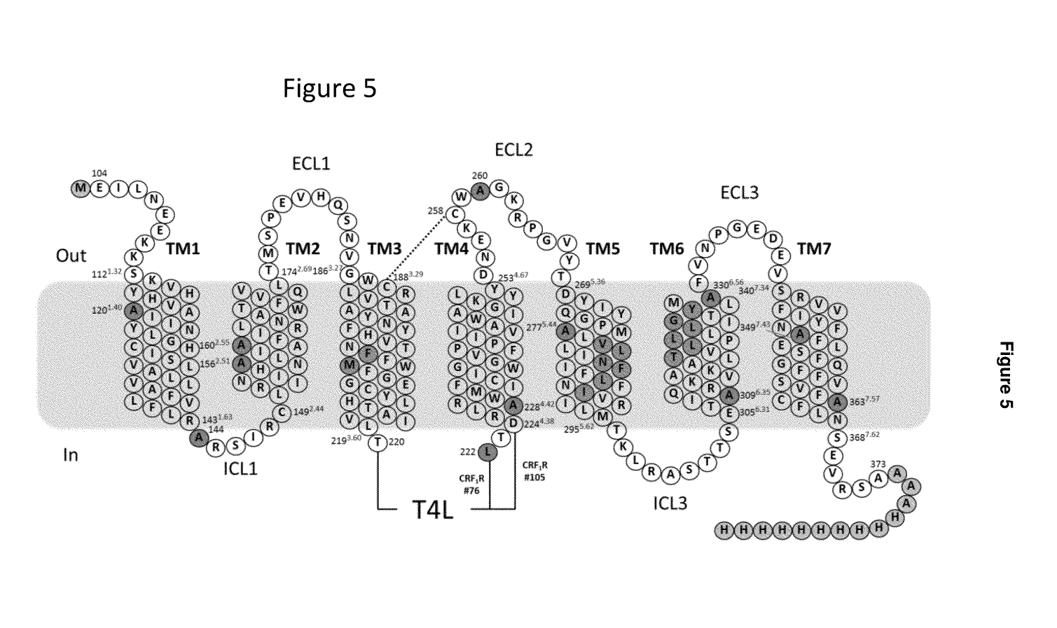

FIG. 5. Crystallization constructs used for CRF₁R structure solution. CRF₁R (residue 104-373) were truncated in both the N- and C-termini, with the site of T4L lysozyme insertion in ICL2 in construct CRF₁R #76 and CRF₁R #105 indicated. Construct CRF₁R #76 comprises SEQ ID NO: 1-T4L-SEQ ID NO: 2. Construct CRF₁R #105 comprises SEQ ID NO: 1-T4L-SEQ ID NO: 3. StaR mutations were colored in grey (V120A, L144A, W156A, 5160A, S222L or S222A [the StaR construct had S222A while the starting T4L-StaR construct had S222L], K228A, F260A, 1277A, Y309A, F330A, S349A and Y363A) and other modified sequence colored in yellow (M104, A(374-376) and H(377-386)). Residues involved in ligand binding were coloured in blue (F203, M206, V279, L280, N283, F284, L287, 1290, T316, L319, L320, L323, G324 and Y327). Disulphide bond between Cys258 on ECL2 and Cys188^{3.29} at the top of TM3 was indicated with a dotted line. The first and last residue in each of the TMs were labeled with the modified Ballesteros-Weinstein numbering system for Class B GPCRs (Wootten, D et al PNAS 2013, 201221585).

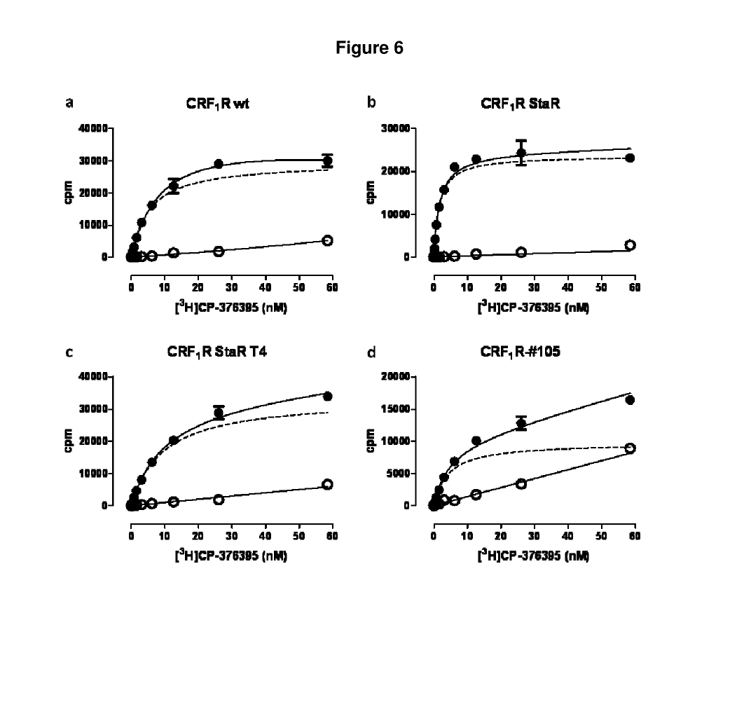

FIG. 6. Saturation binding studies of [³H]CP-376395 to CRF₁R constructs. Representative saturation binding curves of wild-type CRF₁R (a), CRF₁R StaR (b), CRF₁R StaR T4 lysozyme fusion (c), and the truncated receptor CRF₁R-#105 (d) transiently expressed in HEK293T cells. Error bars are derived from standard deviation and calculated from duplicate points (n=2) within a single experiment. Total binding (closed circles) and non-specific binding (open circles) are shown with solid lines and specific binding is shown with dotted lines.

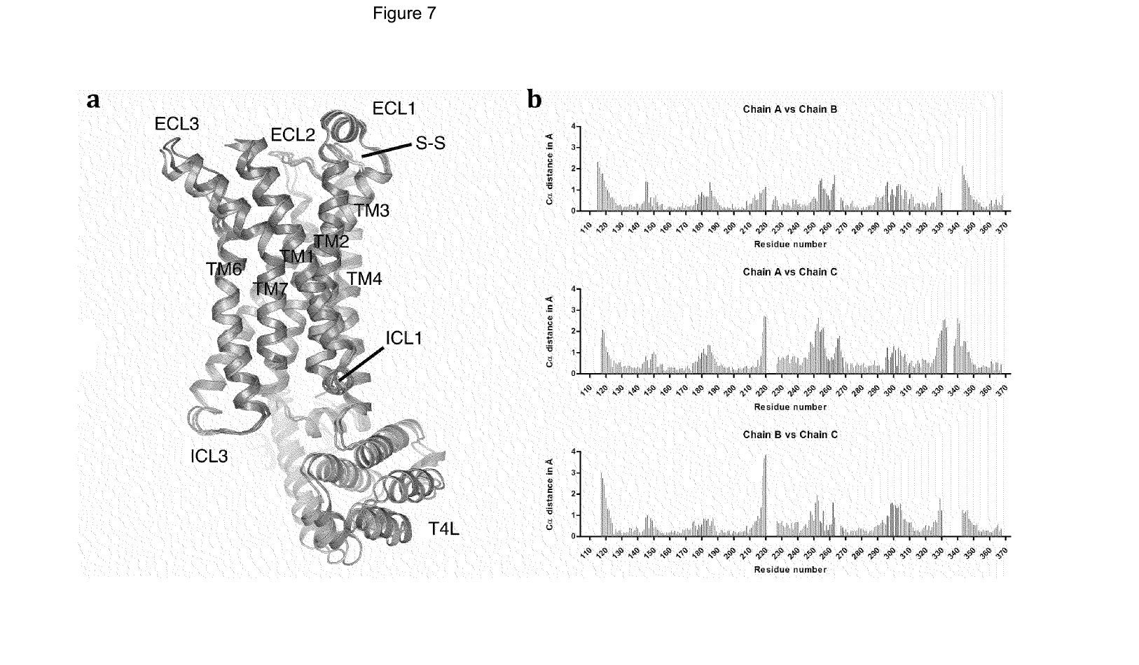

FIG. 7 Superimposition of all 3 chains in the asymmetric unit. (a) The receptor chain B and C were superimposed onto chain A with the CCP4 program Superpose (Krissinel and Henrick, 2004) using the following stretch of residues: Y124^{1.44}-F141^{1.61}, I153^{2.48}-F170^{2.65}, V191^{3.32}-M206^{3.47}, C233^{4.47}-W246^{4.60}, D269^{5.36}-L287^{5.54}, R310^{6.36}-L323^{6.49} and L351^{7.45}-C364^{7.58}. These residues were chosen from the individual TMs where they showed the least movements between the receptor chains from an initial superimposition using the whole receptor. (b) The Ca distance (in Å) between equivalent residues between the receptor chains were plotted against the residue number (104-373) used in the CRF₁R construct. Residues not commonly observed between both chains were not included in the calculation. The plot showed that maximum movement is observed with the first 3 helical turns (extracellular side) of TM1 between each receptor chains, the flexibility of which is also observed in other published Class A GPCR structures, and the extracellular side of TM6 and TM7. Chain A and B are more similar to each other than they are to chain C due to difference in crystal contact environment. For clarity purpose in the comparison with chain C residues 224-226 have been removed due to very large variation in Ca distance as a result of the difference in orientation of the T4L fusion. T4L has not been included in the calculation of this plot.

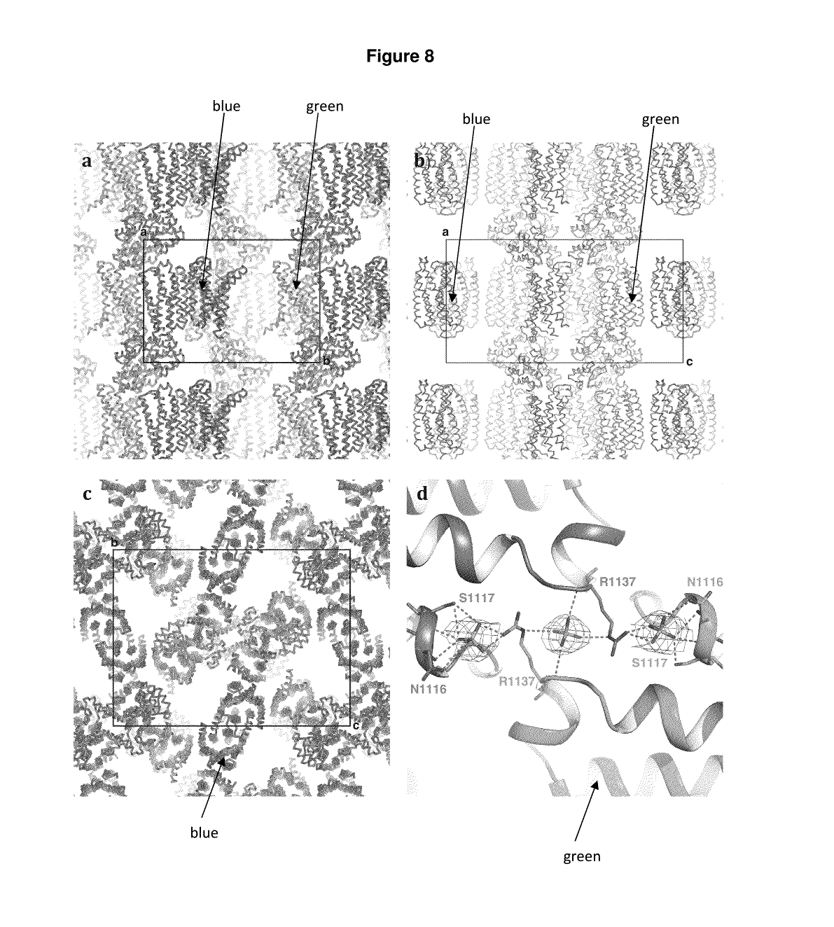

FIG. 8. Packing of the crystal lattice. View of the crystal lattice along the ab (a), ac (b) and be (c) plane. Chain A is in green, chain B in grey and chain C in blue. (d) The crystal lattice consists of layers of receptor molecules (see (b)) and crystal contacts between the layers are mediated by contacts created from the T4L fusion. The T4Ls between two receptor-T4L fusion form a dimer interface with 2 Arg side chains (R1137) intercalated between 3 sulphate ions present in the crystallization buffer. The sulphate ions are stabilized by contacts with S1137 and N1116. 2Fo-Fc density is shown in blue and contoured at 1σ.

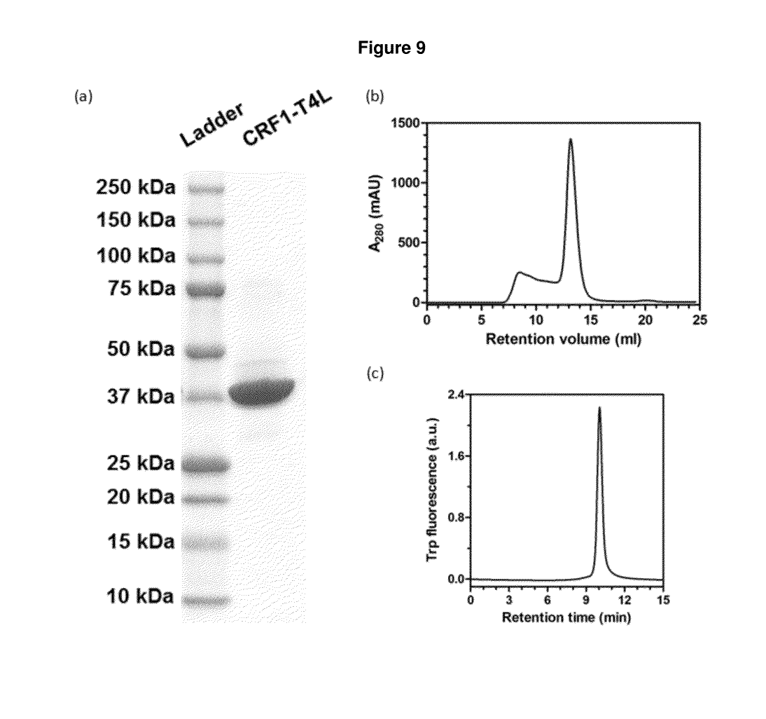

FIG. 9. Biochemical analysis of CRF₁R-T4L. Purified CRF1 was characterized by SDS-PAGE (a) which showed that protein was >98% pure. A symmetrical peak on the elution profile on preparative size exclusion chromatography (b) showed that protein was monodisperse. The monodispersity was maintained after sample was concentrated for crystallization, as monitored by FSEC using intrinsic tryptophan fluorescence (c).

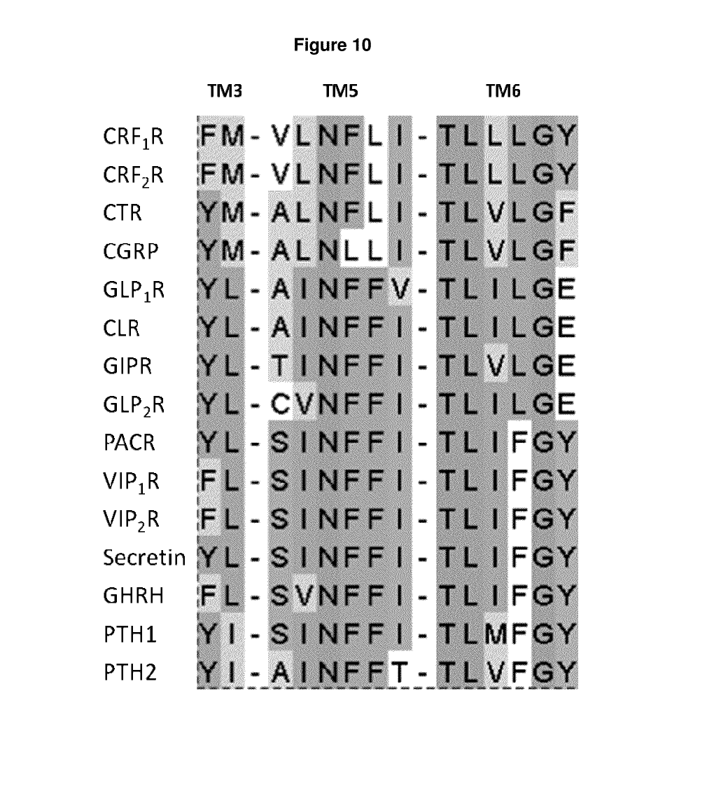

FIG. 10. Sequence conservation of the small organic molecule binding pocket in Class B GPCR. Sequence alignment of the amino acids at a distance of 5 Å from the CP-376395 to the corresponding amino acids in other Class B GPCR. CRF₁R and CRF₂R correspond to SEQ ID NO:4; CTR corresponds to SEQ ID NO:5; CGRP corresponds to SEQ ID NO:6; GLP₁R corresponds to SEQ ID NO:7; CLR corresponds to SEQ ID NO:8; GIPR corresponds to SEQ ID NO:9; GLP₂R corresponds to SEQ ID NO:10; PACR corresponds to SEQ ID NO:11; VIP₁R and VIP₂R correspond to SEQ ID NO:12; Secretin corresponds to corresponds to SEQ ID NO:13; GHRH corresponds to SEQ ID NO:14; PTH1 corresponds to SEQ ID NO:15; and PTH2 corresponds to SEQ ID NO:16.

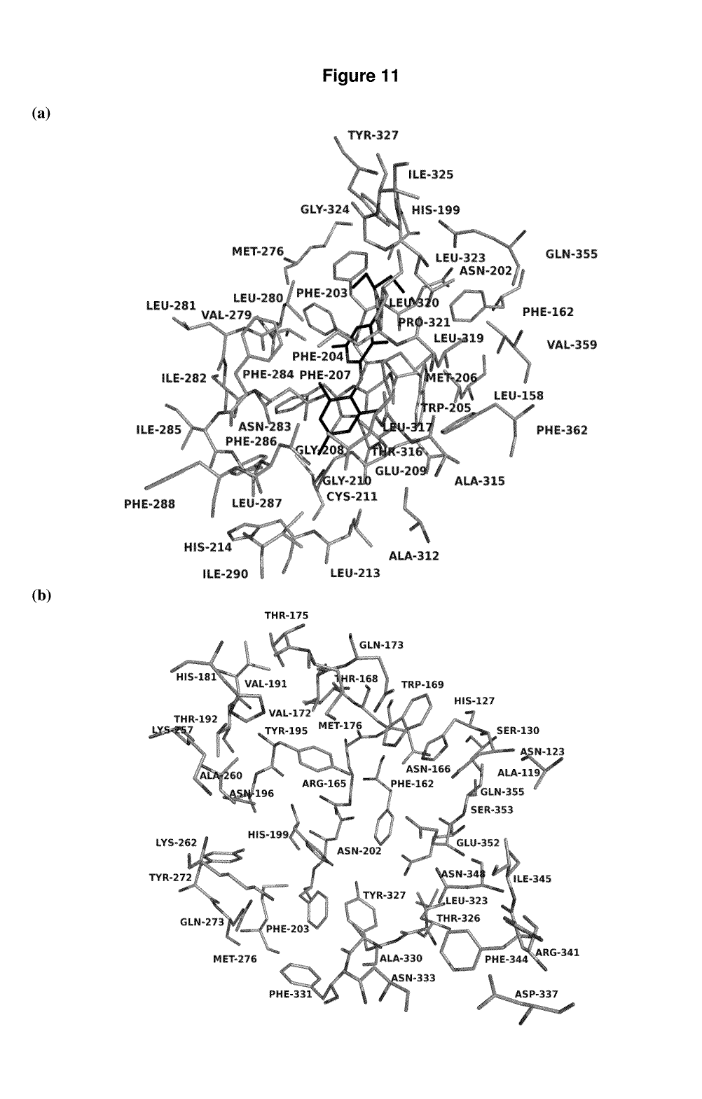

FIG. 11. Sequence (a) Amino acids (in grey) at a distance of 7 Å from CP-376395 (in black). (b) Selection of amino acids potentially near the peptide orthosteric binding site.

FIG. 12. DNA and protein sequence of wild-type CRF₁R. Signal sequence (translated, but cleaved off after protein inserted in membrane) highlighted in bold C-terminal tag underlined. The DNA sequence of wild-type CRF₁R corresponds to SEQ ID NO:17 and the amino acid sequence corresponds to SEQ ID NO:18.

FIG. 13. DNA and protein sequence of CRF₁RNe12.2 StaR. Stabilising mutations highlighted in grey (V120A, L144A, W156A, S160A, S222A, K228A, F260A, I277A, Y309A, F330A, S349A, Y363A); Signal sequence (translated, but cleaved off after protein inserted in membrane) highlighted in bold; C-terminal tag underlined. The DNA sequence of CRF₁RNe12.2 StaR corresponds to SEQ ID NO:19 and the amino acid sequence corresponds to SEQ ID NO:20.

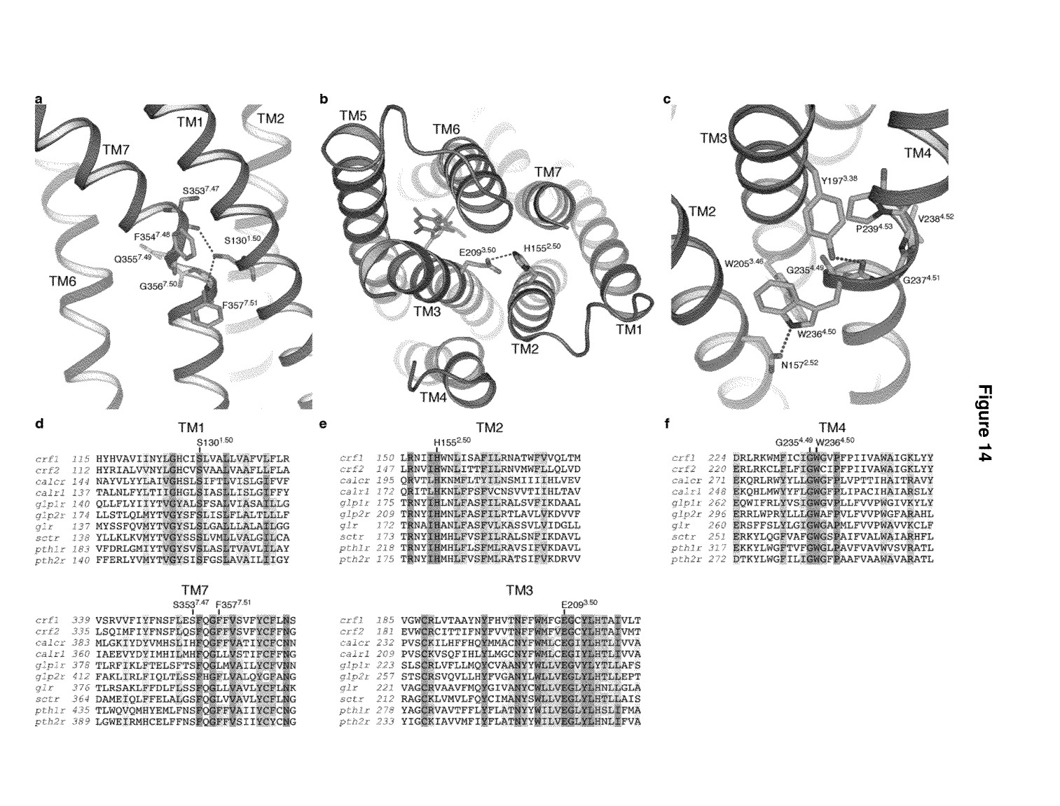

FIG. 14. In a-c, CRF₁R is shown in ribbons with interacting residues shown as sticks. Hydrogen bonds are indicated as dotted lines. CP-376395 is shown in sticks. d-f, Sequence alignments of TM helices of a selected set of human Class B GPCRs involved in the interactions shown in a-c. Highly conserved residues are highlighted in grey, while variable amino-acids are on a white background. For TM1, crf1 corresponds to SEQ ID NO:21, crf2 corresponds to SEQ ID NO:22, calcr corresponds to SEQ ID NO:23, calr1 corresponds to SEQ ID NO:24, glp1r corresponds to SEQ ID NO:25, glp2r corresponds to SEQ ID NO:26, glr corresponds to SEQ ID NO:27, sctr corresponds to SEQ ID NO:28, pth1r corresponds to SEQ ID NO:29, and pth2r corresponds to SEQ ID NO:30. For TM2, crf1 corresponds to SEQ ID NO:31, crf2 corresponds to SEQ ID NO:32, calcr corresponds to SEQ ID NO:33, calr1 corresponds to SEQ ID NO:34, glp1r corresponds to SEQ ID NO:35, glp2r corresponds to SEQ ID NO:36, glr corresponds to SEQ ID NO:37, sctr corresponds to SEQ ID NO:38, pth1r corresponds to SEQ ID NO:39, and pth2r corresponds to SEQ ID NO:40. For TM4, crf1 corresponds to SEQ ID NO:41, crf2 corresponds to SEQ ID NO:42, calcr corresponds to SEQ ID NO:43, calr1 corresponds to SEQ ID NO:44, glp1r corresponds to SEQ ID NO:45, glp2r corresponds to SEQ ID NO:46, glr corresponds to SEQ ID NO:47, sctr corresponds to SEQ ID NO:48, pth1r corresponds to SEQ ID NO:49, and pth2r corresponds to SEQ ID NO:50. For TM7, crf1 corresponds to SEQ ID NO:51, crf2 corresponds to SEQ ID NO:52, calcr corresponds to SEQ ID NO:53, calr1 corresponds to SEQ ID NO:54, glp1r corresponds to SEQ ID NO:55, glp2r corresponds to SEQ ID NO:56, glr corresponds to SEQ ID NO:57, sctr corresponds to SEQ ID NO:58, pth1r corresponds to SEQ ID NO:59, and pth2r corresponds to SEQ ID NO:60. For TM3, crf1 corresponds to SEQ ID NO:61, crf2 corresponds to SEQ ID NO:62, calcr corresponds to SEQ ID NO:63, calr1 corresponds to SEQ ID NO:64, glp1r corresponds to SEQ ID NO:65, glp2r corresponds to SEQ ID NO:66, glr corresponds to SEQ ID NO:67, sctr corresponds to SEQ ID NO:68, pth1r corresponds to SEQ ID NO:69, and pth2r corresponds to SEQ ID NO:70.

### EXAMPLE 1

### Structure of the Corticotropin-Releasing Factor Receptor 1—a Class B GPCR

### Introduction

G protein-coupled receptors (GPCRs) transmit extracellular signals across cell membranes and can be classified into three families (A, B, and C) based on sequence similarity¹. Class B GPCRs include receptors for peptides such as secretin, glucagon, glucagon-like peptide, calcitonin and parathyroid peptide hormone and have been studied as drug targets in the treatment of various diseases, including diabetes, osteoporosis, depression and anxiety. They feature an N-terminal extracellular domain (ECD) involved in peptide-binding and a seven transmembrane α-helices containing transmembrane domain (TMD). Recently determined structures of Class A receptors have greatly advanced our understanding of the function of GPCRs at a molecular level. However, structural information on Class B receptors is currently limited to the ECD and no structure of a Class B TMD, the main target for small-molecule drugs², has been determined to date. Here we report the crystal structure of the TMD of the human corticotropin-releasing factor receptor 1 (CRF₁R)³, a Class B GPCR essential for the stress-induced activation of the hypothalamic-pituitary-adrenal axis, in complex with the non-peptide antagonist CP-376395⁴. The structure reveals significant differences to those of Class A receptors. The extracellular half of the receptor assumes a very open conformation, presumably to allow binding of the large ECD-peptide complex. Furthermore, in contrast to Class A GPCRs where the ligand-binding sites are located close to the extracellular boundaries of the receptors, in CRF₁R the antagonist binds in a hydrophobic pocket located deep in the cytoplasmic half of the receptor. This structure provides new insight into the architecture of Class B GPCRs and may aid in the design of novel therapeutics.

### Results and Discussion

To obtain a structure of CRF₁R, we generated a thermostabilized receptor (StaR) that preferentially adopts the inactive conformation using a conformational thermostabilization approach⁵, previously employed to determine the structures of GPCRs (Table 1). This StaR contained twelve amino-acid substitutions, none of which were located in or adjacent to the ligand-binding site. To facilitate crystallization, both termini were truncated, removing the ECD and amino-acids beyond transmembrane helix 7. Additionally, intracellular loop (ICL) 2 was replaced with T4-lysozyme (T4L) (FIG. 1). Both the full-length StaR and the construct used for crystallization showed similar affinity for the 2-aryloxy-4-alkylaminopyridine CP-376395 to the wild-type receptor (Table 2). The structure was solved by molecular replacement to 2.97 Å with R_{work} and R_{free} of 0.240 and 0.263, respectively. The structures of the three receptors in the asymmetric unit, labeled A-C, were very similar with the exception of the T4L-insertion in molecule C, which was excluded from the model owing to weak electron density (FIGS. 7 and 8, 15). The following discussion is based on molecule C, as there was continuous density for all but a few residues at the termini.

The corefold of CRF₁R features seven transmembrane helices (TM1-TM7) in a generally similar arrangement to those observed in previously determined GPCR structures (FIG. 1). The loops connecting these helices do not feature any secondary-structure except for extracellular loop (ECL) 1, which folds into a short α-helix parallel to the membrane. A helical structure of ECL1 has been proposed in an NMR study on the closely related parathyroid hormone 1 receptor and might therefore be an architectural feature conserved among Class B GPCRs. ECL2 is anchored to the extracellular tip of TM3 by a disulfide bond between Cys188^{3.29} (Class B numbering system in superscript; see above) and Cys258, two fully conserved residues in human, rat and mouse Class B receptors. A similar disulfide-mediated link of ECL2 to TM3 was found in the structures of most Class A GPCRs solved to date and has been identified to be important for agonist-induced receptor activation in the Class B GPCR glucagon-like peptide 1 receptor.

Comparison of the structures of CRF₁R with previously solved GPCRs provides insight into the architectural differences between Class A and Class B receptors. Unlike the compact overall architecture of Class A GPCRs, CRF₁R adopts a pronounced V-shape, presenting a large, polar cavity accessible from the extracellular side (FIG. 1b and d). We illustrate the structural differences using the dopamine D₃ receptor (D₃R)⁶ as a representative of Class A GPCRs, because its overall fold is closest to that of CRF₁R (Table 4). Superposition of these two receptors shows striking differences in the arrangement of TM7 and smaller, but substantial differences in TM6 (FIG. 2). For these two helices the root mean square deviation (RMSD) of the backbone atoms is 3.2 Å and 4.5 Å, respectively. In contrast, the other helices superimpose better with RMSDs between 1.4 Å and 2.7 Å (Table 4). As a result of a sharp kink at Gly356^{7.50}, the extracellular half of TM7 projects far out of the helical bundle. Despite a similar kink found in D₃R, the extracellular end of TM7 in CRF₁R is located approx. 10 Å further away from the long axis of the molecule (Table 5). TM6 adopts a similar shape in CRF₁R and D₃R, however in CRF₁R it is shifted away from TM5 and from the long axis of the receptor. In addition, the extracellular end of TM6 is shorter than in D₃R by two helical turns, which limits the interactions between TM5 and TM6 to the cytoplasmic regions. Significant structural variation has been observed in the extracellular regions of TM1 in the Class A GPCR structures reported to date. In CRF₁R, its slightly bent N-terminal portion packs against the extracellular end of TM7 and hence follows a similar trajectory away from the center of the receptor. Highly conserved Ser130^{1.50} stabilizes the kink in TM7 through hydrogen bonds to the backbone at Phe357^{7.51}(3.1 Å) and Ser353^{7.47}(3.0 Å, FIGS. 14a and d). A similar interaction is found in Class A receptors where a conserved asparagine at position 1.50 (Ballesteros-Weinstein-numbering⁷), located one helical turn down relative to Ser130^{1.50} in CRF₁R, binds to the backbone of TM7. While in most Class A receptors the extracellular tip of TM2 is kinked towards TM1 caused by a slight unwinding of the helix, in CRF₁R this helix is more or less straight, thereby contributing to the opening of the extracellular side of the receptor. The open structure of the receptor may relate to the way the large peptide agonistin complex with the ECD interacts with the TMD.

Despite the limited sequence similarity between Class A and Class B GPCRs, signaling through both receptor classes is through the same effector proteins. The comparison of CRF₁R with D₃R revealed that in contrast to their extracellular portions their cytoplasmic parts superimpose well (FIG. 2d, Table 5). Specifically, the C-terminal halves of TM3 and TM5, which have been found to interact with Gas in the structure of the β₂-adrenergic receptor-Gs complex, adopt very similar conformations in CRF₁R and D₃R. Further, despite the lack of a native ICL2 due to the T4L-insertion between TM3 and TM4 in the present structure, the relative arrangement of the intracellular half of TM4 with respect to the intracellular end of TM3 is very similar to that found in D₃R. This would bring ICL2 into a similar position to Class A receptors, thus allowing receptor-G-protein interaction. Even though TM6 in CRF₁R is shifted outward, owing to its bent shape and an extra helical turn at its N-terminus, its cytoplasmic endpoints towards TM3 and the long axis of the receptor in a similar fashion as in the structures of D₃R and other Class A receptors solved in an inactive state. This also suggests that the conformation in the CRF₁R crystal structure represents the inactive state.

In Class A GPCRs, a conserved salt bridge connects TM6 to TM3 in the inactive state. The sequence motifs for this ‘ionic lock’ are absent in Class B receptors. Instead, biochemical data suggests interaction of His155^{2.50} and Glu209^{3.50} to play an essential role in activation. In our structure, these two side-chains are within hydrogen-bonding distance (3.1 Å), forming a potentially important functional micro-switch (FIGS. 14b and e). In Class B GPCRs, a conserved sequence motif, termed GWG×P-motif, is found in TM4. The CRF₁R structure reveals a network of interactions centered around this highly conserved Trp236^{4.50} that links TM4 to TM2 and TM3 (FIGS. 14c and f). TM4 slightly unwinds at Gly235^{4.49}, resulting in a bulge in the helix and Trp236^{4.50} protruding towards TM3 and TM2. This side-chain hydrogen bonds (2.8 Å) to the side-chains of Asn157^{2.52} in TM2 and forms an edge-to-face interaction (′T-stack) with Trp205^{3.46} in TM3. The TM4-TM3 interaction is strengthened by a hydrogen bond (2.8 Å) between the side-chain of Tyr197^{3.38} to the main-chain carbonyl of Trp236^{4.50} and hydrophobic contacts of Gly235^{4.49} with Trp205^{3.46} and Pro239^{4.53} with Tyr197^{3.38}. How these interactions are involved in receptor function is not immediately clear from the structure and requires further investigation.

Unexpectedly, we found strong electron density for the small-molecule antagonist CP-376395 in a pocket located deep into the cytoplasmic half of the receptor (FIG. 3). The position of this binding site is remarkable, as it is approx. 18 Å away from the center of the large hydrophilic cavity presented to the extracellular side, the putative peptide agonist-binding site, and between approx. 13 Å and 23 Å away from the centers of mass of the small-molecule antagonists and agonists found in the structures of the Class A GPCRs determined to date (FIG. 3f). In an orientation approx. perpendicular to the plane of the membrane, CP-376395 binds in a predominantly hydrophobic binding site defined by residues of TM3, TM5 and TM6. A key interaction, and the only polar contact to the ligand, is mediated by the side-chain of highly conserved Asn283^{5.50} in TM5, which forms a hydrogen bond (2.9 Å) with the pyridine nitrogen. Disruption of this important polar interaction by mutating Asn283^{5.50} to alanine resulted in complete loss of ligand binding (Table 6). Additional interactions to the pyridine core are provided by Met206^{3.47} and Val279^{5.46}. Extensive hydrophobic contacts to the aryloxy moiety are mediated by the side-chains of Phe284^{5.51}, Leu287^{5.54}, Ile290^{5.57}, Tyr316^{6.42}, Leu319^{6.45} and Leu320^{6.46}. The Alkyl substituent of the exocyclic secondary amine interacts with Gly324^{7.50} and the side-chains of Phe203^{3.44}, Leu280^{5.47}, Leu323^{6.40} and Tyr327^{6.53}. To validate our structure, we individually substituted the fourteen amino-acids in the binding site with alanine (Table 6). With the exception of Leu319^{6.45}, all of these mutations resulted in a significant reduction of antagonist binding with negligible or no binding detected for Phe203^{3.44}Ala, Leu280^{5.47}Ala and Asn283^{5.50}Ala. At the bottom of the putative orthosteric site, Arg165^{2.60}, His199^{3.40}, Met276^{5.43} and Gln355^{7.49} form a layer of inward-pointing side-chains just above the antagonist binding site (FIG. 3d). Mutation of His199^{3.40} and Met276^{5.43} have been shown to impair non-peptide antagonist binding in CRF₁R. While not interacting with CP-376395 directly, H199^{3.40} forms a hydrogen bond (2.8 Å) with Tyr327^{6.53} and Met276^{5.43} packs against Phe203^{3.44}, thereby potentially supporting the positioning of these two aromatic side-chains important for ligand binding. Substitution of highly conserved Gln355^{7.49} with alanine did not affect CP-376395 binding (Table 6). However, mutation of the equivalent residue in other Class B GPCRs was shown to reduce peptide-agonist binding and receptor activation and might therefore be involved in agonist-binding in the orthosteric site. The cytoplasmic half of TM6 was shown to play a key role in receptor activation by moving away from the core of the TM bundle. In our structure, CP-376395 keeps the receptor in an inactive conformation by making extensive contacts with TM3, TM5 and TM6. Specifically, the ligand binds to residues in TM6 on both sides of the kink induced by Pro321^{6.47} and Gly324^{6.50}, thereby tethering the cytoplasmic half of TM6 to TM3 and TM5.

Access to this binding site from the extracellular side is restricted to a small channel by the side-chains of Phe203^{3.44} and Tyr327^{6.53} (FIG. 3e). Based on the present structure, there are two possibilities for CP-376395 to reach the binding site: (i) Rearrangement of residues at the top of the binding site, in particular of Phe203^{3.44} and Tyr327^{6.53}, through side-chain rotamer changes and/or outward shifts of portions of TM3, TM5 or TM6 increase the size of the opening of the binding site towards the putative orthosteric site allowing the ligand to bind from the extracellular side. (ii) The binding site opens laterally towards the membrane through rearrangements of TM5 and TM6 allowing diffusion of the highly hydrophobic ligand into the binding site after partitioning into the membrane.

In addition, the antagonist-binding site is separated from the interior of the membrane merely by a single layer of side-chains provided by amino-acids in TM5 and TM6 and, hence, lateral opening of the binding site would require only minor rearrangements in the receptor. Further studies are needed to elucidate the precise mechanisms of antagonist binding.

The structure of the inactive state of CRF₁R reported here provides valuable insight into the overall architecture of Class B GPCRs as well as into the molecular basis of Class B receptor antagonism.

### Tables

TABLE 1
Thermal stability of CRF₁R

| Construct | Mean Tm (° C.) |
|---|---|
| CRF₁R wild type | 18.4 (2.0) |
| CRF₁R StaR | 44.7 (2.2) |
| CRF₁R StaR T4 | 37.5 (0.7) |
| CRF₁R-#105 | 35.7 (1.1) |

Thermal stability of CRF₁R constructs measured using [³H]CP-376395 binding and solubilised in DDM. Data are shown as the mean of four independent experiments with standard deviation displayed in parentheses.

TABLE 2
K_{d} analysis of [³H]CP-376395 binding to CRF₁R constructs

| Construct | Mean K_{d} [³H]CP-376395 (nM) |
|---|---|
| CRF₁R wild type | 7.5 (2.4) |
| CRF₁R StaR | 1.5 (0.1) |
| CRF₁R StaR T4 | 6.7 (2.5) |
| CRF₁R-#105 | 5.0 (0.8) |

Affinity measurements calculated from saturation binding studies with [³H]CP-376395 to membranes isolated from transiently expressed HEK293T cells. Data are shown as the mean of three independent experiments with standard deviation displayed in parentheses.

TABLE 3
Data collection and refinement statistics

| | Conventional data | | | Microdiffraction | | |
|---|---|---|---|---|---|---|
| Processing Method | assembly method | | | assembly method | | |
| Data collection | | | | | | |
| Number of crystals | 35 | | | 35 | | |
| Space group | P22₁2₁ | | | P22₁2₁ | | |
| Cell dimensions | | | | | | |
| a, b, c (Å) | 86.6 | 124.0 | 166.8 | 86.6 | 124.0 | 166.8 |
| α, β, γ (°) | 90.0 | 90.0 | 90.0 | 90.0 | 90.0 | 90.0 |

| | | |
|---|---|---|
| Number of reflections | 80784 | 81748 |
| measured | | |
| Number of unique | 29375 | 32141 |
| reflections | | |
| Resolution (Å)* | 34.14-3.15 (3.34-3.15) | 34.15-2.97 (3.14-2.97) |
| R_{merge} | 0.154 (0.722) | 0.136 (0.442) |
| Mean I/sd(I) | 7.6 (1.9) | 6.7 (1.9) |
| Completeness (%) | 93.3 (93.2) | 86.3 (68.2) |
| Redundancy | 2.8 (2.8) | 2.5 (2.0) |
| Refinement | | |
| Resolution (Å) | | 34.15-2.97 |
| Number of reflections | | 32124 (2367) |
| (test set) | | |
| R_{work}/R_{free} | | 0.2402/0.2630 |
| Number of atoms | | |
| All | | 8912 |
| Proteins | | 8477 |
| Ligand | | 72 |
| Others (Lipids, ions, | | 363 |
| waters) | | |
| Average B value (Å²) | | |
| All | | 62.6 |
| CRF₁R | | 63.2 |
| T4L lysozyme | | 61.3 |
| Ligand | | 51.0 |
| Others (Lipid, ion, | | 63.4 |
| water) | | |
| RMSD | | |
| Bond lengths (Å) | | 0.012 |
| Bond angles (°) | | 1.359 |
| Ramachandran statistics | | |
| Favored regions (%) | | 98.1 |
| Allowed regions (%) | | 1.9 |
| Outliers (%) | | 0 |
| Molprobity overall score | | 98.5 |
| (percentile) | | |

*Value in parenthesis indicates highest resolution shell value

TABLE 4
RMSD between the crystallographic structure of CRF₁R and several Class A GPCR
structures calculated on the protein backbone of the global common TM region*

| | | RMSD* | | | | | | | |
|---|---|---|---|---|---|---|---|---|---|
| | | All | | | | | | | |
| Receptor | PDB ID | TMs | TM1 | TM2 | TM3 | TM4 | TM5 | TM6 | TM7 |
| Rhodopsin | 1F88 | 3.4 | 2.4(0.9) | 3.1(2.3) | 3.2(1.2) | 3.2(1.9) | 3.1(1.9) | 3.2(1.3) | 5.2(1.6) |
| β₁ adrenergic | 2VT4 | 3.2 | 3.0(1.0) | 2.9(2.1) | 1.8(0.7) | 2.6(1.8) | 2.4(1.6) | 3.4(1.3) | 5.5(1.5) |
| β₂ adrenergic | 2RH1 | 3.1 | 2.6(0.9) | 3.0(2.2) | 1.7(0.8) | 2.5(1.6) | 2.4(1.6) | 3.3(1.4) | 5.4(1.7) |
| β₂ adrenergic | 3SN6 | 3.2 | 2.3(1.2) | 3.0(2.1) | 1.4(0.6) | 2.7(1.6) | 2.5(1.6) | 4.1(2.0) | 5.6(1.9) |
| Adenosine A_{2A} | 2YDV | 2.7 | 2.2(0.6) | 2.1(1.9) | 1.8(0.9) | 2.8(1.7) | 2.9(1.7) | 2.4(1.3) | 4.3(1.4) |
| Adenosine A_{2A} | 3PWH | 2.8 | 2.0(0.5) | 2.2(1.9) | 1.7(1.1) | 3.1(1.9) | 2.5(1.8) | 3.2(1.3) | 4.5(1.2) |
| CXCR4 | 3ODU | 3.2 | 2.3(1.3) | 2.8(1.7) | 1.8(0.7) | 3.1(2.0) | 2.9(1.8) | 3.1(1.3) | 5.6(1.9) |
| Dopamine D₃ | 3PBL | 2.7 | 1.9(0.7) | 2.6(2.1) | 1.4(0.6) | 2.7(1.8) | 2.2(1.6) | 3.2(1.1) | 4.6(1.1) |
| Histamine H₁ | 3RZE | 2.9 | 1.8(0.8) | 2.7(1.9) | 1.5(1.0) | 3.0(2.2) | 2.8(1.6) | 3.1(1.3) | 5.0(1.4) |
| Muscarinic M₂ | 3UON | 3.1 | 2.7(1.0) | 2.9(2.0) | 1.6(0.6) | 2.5(1.4) | 3.0(1.4) | 3.3(1.1) | 5.0(1.0) |
| Muscarinic M₃ | 4DAJ | 3.2 | 1.9(0.8) | 2.8(2.1) | 1.5(0.8) | 2.6(1.6) | 3.5(1.6) | 3.4(1.7) | 5.5(1.2) |
| S1P₁ | 3V2Y | 3.2 | 3.0(0.8) | 2.0(0.7) | 1.8(1.0) | 3.6(1.7) | 2.3(0.7) | 2.6(1.2) | 5.9(1.9) |
| κ opioid | 4DJH | 3.2 | 2.6(1.0) | 3.0(1.2) | 2.1(1.3) | 3.5(2.4) | 2.9(1.8) | 3.5(1.1) | 4.8(1.1) |
| μ opioid | 4DKL | 3.0 | 2.2(0.9) | 2.9(1.1) | 2.1(1.1) | 3.1(2.3) | 3.2(1.8) | 3.2(1.1) | 4.4(1.1) |
| δ opioid | 4EJ4 | 3.2 | 2.3(0.8) | 3.1(1.4) | 2.1(1.2) | 3.3(2.2) | 3.1(1.9) | 3.6(1.2) | 4.5(1.1) |
| ORL-1 | 4EA3 | 3.0 | 2.1(0.9) | 3.0(1.3) | 2.0(1.0) | 3.0(2.2) | 3.0(1.7) | 3.2(1.2) | 4.6(1.2) |
| PAR-1 | 3VW7 | 3.7 | 2.4(1.3) | 3.5(1.5) | 2.7(1.4) | 3.9(2.0) | 3.5(0.9) | 3.8(2.0) | 5.8(2.0) |
| NTSR₁ | 4GRV | 2.8 | 2.2(0.8) | 2.6(1.9) | 1.2(1.0) | 2.8(1.8) | 3.2(1.8) | 3.1(1.4) | 4.3(1.4) |

*Backbone RMSD values were calculated after a global superposition using a core TM region shared by Class A GPCRs and CRF₁R as defined by CRF₁R residues 119-143, 150-176, 186-218, 227-247, 269-294, 312-332, and 343-365, corresponding to the Class A Ballesteros-Weinstein residues 1.35-1.59, 2.38-2.64, 3.23-3.55, 4.41-4.61, 5.40-5.65, 6.33-6.53, and 7.33-7.55. Backbone RMSD values in brackets were calculated after local superposition of the individual TMs. Chain C was used for CRF₁R, while chain A for the Class A GPCRs.

TABLE 5
Distances between helix termini in CRF₁R and D₃R

Cα-distance in Å (CRF₁R-D₃R)

| | | |
|---|---|---|
| TM helix | intracellular side | extracellular side |

| | | | | |
|---|---|---|---|---|
| TM1 | 0.7 | (R143-K56) | 3.3 | (A120-A33) |
| TM2 | 1.7 | (L150-T63) | 5.3 | (T175-L89) |
| TM3 | 1.5 | (V218-V133) | 0.8 | (G186-I101) |
| TM4 | 1.1 | (R227-V150) | 4.8 | (A247-L169) |
| TM5 | 0.3 | (L294-L215) | 2.3 | (I271-Y191) |
| TM6 | 3.2 | (Q308-R323) | 5.7 | (V332-L347) |
| TM7 | 5.3 | (F365-T385) | 10.5 | (V343-E363) |

Superposition of D₃R on CRF₁R was done as described in Methods.

TABLE 6
The effect of alanine mutagenesis on
the binding of [³H]CP-376395 to CRF₁R

| CRF₁R | Normalised % wt |
|---|---|
| mutation | \|³H]CP-376395 binding |
| F203A(3.44) | 1 |
| M206A(3.47) | 8 |
| V279A(5.46) | 18 |
| L280A(5.47) | 1 |
| N283A(5.50) | 0 |
| F284A(5.51) | 60 |
| L287A(5.54) | 5 |
| I290A(5.57) | 5 |
| T316A(6.42) | 47 |
| L319A(6.45) | 120 |
| L320A(6.46) | 33 |
| L323A(6.49) | 26 |
| G324A(6.50) | 41 |
| Y327A(6.53) | 8 |
| Q355A(7.49) | 101 |

Solubilised whole cell ligand binding is expressed as a percentage of wild-type CRF₁R total binding. Data has been normalized for expression using eGFP tagged constructs. All mutants showed expression ≧50% of wild-type CRF₁R. Class B Ballesteros numbering for each mutation is shown in parentheses.

### Materials and Methods

### StaR Generation

The CRF₁R StaR was generated using a mutagenesis approach as previously described (Robertson et al, 2011). Mutants were analyzed for thermostability in the presence of the antagonist radioligand [³H]CP-376395. The CRF₁R StaR contained 12 mutations; V120^{1.40}A, L144A, W156^{2.51}A, S160^{2.55}A, S222A, K228^{4.42}A, F260A, I277^{5.44}A, Y309^{6.35}A, F330^{6.56}A, S349^{7.43}A, and Y3637.57A (FIG. 13), one of which (S222A) was removed upon insertion of T4L into ICL2.

### Cell Culture

HEK293T cells were maintained in culture in DMEM supplemented with 10% (v/v) fetal bovine serum (FBS, Sigma-Aldrich) and passaged twice weekly. Cells were transfected with CRF₁R constructs using GeneJuice (Merck Millipore) according to manufacturer's instructions and harvested after 48 hours using PBS supplemented with EDTA-free protease inhibitors (Roche). Membranes for use in radioligand binding assays were prepared as previously described (Robertson et al, 2011).

### Thermostability Measurement

HEK293T cells transiently transfected with CRF₁R constructs were incubated in buffer (50mMTris-HCl pH 7.5, 150mMNaCl, EDTA-free protease inhibitors) with 30 nM [³H]CP-376395, and 120 nM cold CP-376395 (Tocris) for 18 hours at room temperature. Reactions were transferred to ice and all subsequent steps performed at 4° C. Cells were solubilized in 1% (w/v) n-dodecyl-β-D-maltopyranoside (DDM, Affymetrix) for 1 hour and crude lysates cleared by centrifugation at 16.000×g for 15 minutes. Thermostability of the receptor was measured by incubation at varying temperatures for 30 minutes followed by separation of excess and unbound radioligand by gel filtration. Levels of ligand-bound receptor were then determined using a liquid scintillation counter.

### Radioligand Binding

For saturation binding experiments, membranes isolated from HEK293T cells transiently expressing wild-type CRF₁R (15 μg/well), CRF₁R StaR (6 μg/well), CRF₁R StaR with T4L fusion (20 μg/well), and CRF₁R-#105 (20 μg/well) were incubated in buffer (50mMTris-HCl pH 7.5, 150 mM NaCl, 0.1% (w/v) PEI, EDTA-free protease inhibitors) with [³H]CP-376395 (0-60 nM) in the presence or absence of 30 uM cold CP-376395 in a final volume of 500 μl. Final DMSO concentration in each reaction was 5% (v/v). Membranes were incubated for 18 hours at room temperature before being terminated by rapid filtration through 96-well GF/C UniFilter plates pre-soaked in 0.3% (w/v) PEI, followed by washing with PBS with 0.15% (w/v) CHAPS. Plates were dried, 50 μl Ultima Gold-F added per well and bound ligand measured using a Packard Microbeta counter. Data were analyzed using a global fitted one-site binding hyperbola in GraphPad Prism v5 to generate K_{d}. For solubilized whole cell ligand binding experiments, HEK293T cells transiently expressing eGFP-tagged wild-type CRF₁R or single point mutants were treated as described above for thermostability experiments, without the 30 minute heating step. Specific binding was determined by subtracting untransfected controls. Expression of each construct was quantified by eGFP fluorescence of whole cells measured at Ex/Em of 488/520 nm.

### Truncation and Lysozyme Fusion Constructs

A panel of N- and C-terminal truncations of the human CRF₁ receptor was designed based on secondary structure prediction and hydropathy plots. Truncated receptors were expressed HEK293Tcells as C-terminal fusions with eGFP. Receptors were solubilized in 50 mM Tris-HCl pH 8.0, 150 mM NaCl, and 2% (w/v) n-decyl-β-D-maltopyranoside (DM, Affymetrix) and their expression levels and stability was assayed by fluorescence-detection size exclusion chromatography as described (FSEC) (Kawate & Gouaux, 2006). The most suitable construct emerging from this screen comprised residues 104-373, lacking the ECD and residues past the predicted location of TM7. In parallel, a panel of T4L insertions into the predicted locations of ICL2 or ICL3 were analyzed in a similar fashion, identifying the insertion between residues 220 and 222 in ICL2 as the most promising fusion (construct CRF₁R-#76). To obtain the P22₁2₁ crystal form, which enabled structure determination, residues 222 and 223 were later deleted, yielding the final fusion construct CRF₁R-#105 (see below). A schematic representation of the constructs is shown in FIG. 5.

### Expression and Purification

CRF₁R carrying a C-terminal deca-histidine tag was expressed in Trichoplusia ni (High Five) cells in EX-CELL 405 medium (Sigma-Aldrich) supplemented with 10% (v/v) FBS, 1% (v/v) CD lipid concentrate (GIBCO) and 1% (v/v) Penicillin/Streptomycin (PAA Laboratories). Cells were infected at a density of 2×10⁶ cells/ml with 10 ml of baculovirus per liter of culture, corresponding to an approximate multiplicity of infection (moi) of 1. Cultures were grown at 27° C. with constant shaking and harvested 72 hours post infection. Cells were pelleted and washed with 250 ml PBS and stored at −80° C. All subsequent purification steps were carried out at 4° C. unless indicated differently. To prepare membranes, cell were thawed at room temperature and resuspended in 400 ml ice-cold 50 mM Tris-HCl pH 8.0, 500 mM NaCl supplemented with EDTA-free protease inhibitors. The cell suspension was incubated with 0.3 μM CP-376395 for 1 hour to allow the ligand to bind. Cells were disrupted by ultra-sonication and cell debris was removed by centrifugation at 10.000×g. Membranes were collected by ultracentrifugation at 140.000×g, resuspended and stored at −80° C. until further use. Membranes were thawed at room temperature and solubilized with 2% (w/v) DM for 1.5 hours. Insoluble material was removed by ultra-centrifugation and the receptors were immobilized by batch binding to TALON metal-affinity resin (Clontech) for 2 hours. The resin was packed into a XK-16 column (GE Healthcare) and washed with steps of 8 and 30 mM imidazole in 50 mM Tris-HCl pH 8.0, 500 mM NaCl, 0.15% (w/v) DM, and 0.3 μM CP-376395 for a total of 15-20 column volumes before bound material was eluted with 200 mM imidazole. The protein was then concentrated using an Amicon Ultra-15 centrifugal filter unit (Millipore) and subjected to preparative gel filtration in 20 mM Tris-HCl pH 8.0, 150 mM NaCl, 0.15% (w/v) DM, and 0.3 μM CP-376395 on a Superdex200 10/300 GL gel filtration column (GE Healthcare) to remove remaining contaminating proteins and aggregates. It is important to note that in preparations of CRF₁R-#105 significantly more aggregated material was obtained than with CRF₁R-#76. For improved yields and a higher degree of homogeneity the procedure was altered as follows. After elution from the metal affinity resin the buffer was exchanged to 50 mM Tris-HCl pH 8.0, 500 mM NaCl, 0.15% DM, 0.3 μM CP-376395 and 5 mM EDTA by desalting. In addition, the final buffer was supplemented with 1-palmitoyl-2-oleoyl-sn-glycero-3-phospho-(1′-rac-glycerol) (POPG, Avanti Polar Lipids) at a concentration of 0.005% (w/v). Receptor purity was analyzed using SDS-PAGE and mass spectrometry and receptor mono-dispersity was assayed by FSEC monitoring tryptophan fluorescence (FIG. 9). Protein concentration was determined with a NanoDrop spectrophotometer using the receptor's calculated extinction coefficient at 280 nm (ε_{280, calc}=1.6 (mg/ml×cm)⁻¹).

### Crystallization

CRF₁R was crystallized in lipidic cubic phase (LCP) at 22.5° C. The protein was concentrated to 20-30 mg/ml by ultrafiltration and mixed with monoolein (Nu-Check) supplemented with 10% (w/w) cholesterol (Sigma) and 5 μM CP-376395 using the twin-syringe method (Caffrey & Cherezov, 2009). The final protein:lipid ratio was 1:1.5(w/w). With the help of a dispensing robot (Mosquito LCP, TTP Labtech), 40-60 nlboli were dispensed on 96-well Laminex Glass Bases (Molecular Dimensions), overlaid with 0.75 μl precipitant solution and sealed off with LaminexFilm Covers (Molecular Dimensions). 20-30 μm crystals of construct CRF₁R-#76 were obtained in 100 mM Na-citrate pH 5.5, 200 mM Li₂SO₄, 30% (v/v) polyethylene glycol 400, and 0.6 μM CP-376395 and we were able to collect a complete dataset to 3.2 Å by combining data from multiple crystals. The crystals belonged to hexagonal spacegroup P6 and the data featured a 30% off-origin peak in a native Patterson map, indicating translational non-crystallographic symmetry (tNCS). Extensive trials to solve the structure by molecular replacement failed, most likely due to the presence of tNCS. We hypothesized that conformational flexibility in the connection between the receptor and T4L was the cause for the observed pseudo-symmetry in the crystals and that deletion of residues in this part of the CRF₁R-T4L fusion would reduce flexibility of the construct and, hence, enable growth of a different crystal form without tNCS. The resulting construct CRF₁R-#105 (FIG. 5) crystallized in the same conditions as CRF₁R-#76 and 20 μm brick-shaped crystals grew and attained maximum size within 7-10 days. Crystals were flash-frozen in liquid nitrogen without additional cryoprotectant.

### Diffraction Data Collection and Processing

X-ray diffraction data were measured on a Pilatus 6M hybrid-pixel detector at Diamond Light Sourcebeamline I24 using a 5 μm×5 μm microbeam. Crystals displayed isotropic diffraction to beyond 3.0 Å following exposure to an unattenuated beam for 7.5 seconds per degree of oscillation. Consequently, radiation damage was severe and wedges of typically only 2-3 degrees per crystal could be used for data merging. Data from individual crystals were integrated using XDS (Kabsch, 2010) and a complete dataset was compiled using the data collection strategy option of the programMosflm (Leslie & Powell, 2007). Data merging and scaling was carried out with AIMLESS (Evans & Murshudov, 2012; Collaborative Computational Project, Number 4, 1994). The final dataset comprised data from 35 crystals and was scaled to 3.15 Å with a completeness of 93.3% overall using a combination of isotropic resolution cut-off criteria such as </>/<σ/> and R_{merge.} Crystals belonged to orthorhombic spacegroup P22₁2₁ with unit cell dimension of a=86.6 Å, b=124.0 Å, c=166.8 Å, á=â=ã=90°. Using the microdiffraction assembly method as described previously (Hanson et al, 2012) we were able to extend the resolution of the dataset to 2.97 Å. Briefly, data from each crystal were split into wedges of reflection observations corresponding to 1° of oscillation and then scaled individually to a medium-resolution (4.3 Å) reference dataset, collected from a single crystal, using XSCALE (Kabsch, 2010) without merging reflections. Initially, as rejection criterion for reflections, the peak profile correlation threshold was set to zero and increased in increments of 1% until all reflection observations could be scaled with an R_{merge}lower than 14%. The resulting multi-record reflection file was then scaled using AIMLESS. Data collection statistics for both methods are presented in Table 3. For subsequent structure solution and refinement the data processed using the micro-diffraction assembly method was used.

### Structure Solution and Refinement

Cell content analysis using the Matthews volume (Matthews, 1968) suggested the presence of three copies of receptor-T4L fusion in the asymmetric unit, resulting in a solvent content of 57%. The structure was solved by molecular replacement (MR) with the program Phaser (McCoy et al, 2007, Collaborative Computational Project, Number 4, 1994) using two independent search models, T4L from the adenosine A_{2A} receptor structure (PDB ID 3EML) and a truncated version (TM helices only, no loops) of the dopamine D₃ receptor (PDB ID 3PBL). Solutions were found for two out of the three T4L copies, which were subsequently fixed to locate three copies of the truncated receptor. Manual model building was done in COOT (Emsley et al, 2010) using sigma-A weighted 2Fₒ-F_{c}, Fₒ-F_{c} as well as a simulated-annealing composite omit maps calculated using Phenix (Adams et al, 2010). Initial refinement was carried out with REFMAC5 (Murshudov et al, 2011, Collaborative Computational Project, Number 4, 2007) using the maximum-likelihood restrained refinement protocol in combination with the jelly-body method and imposing tight non-crystallographic symmetry (NCS) restraints. Later stages of the refinement were performed with Phenix using a combination of simulated annealing, positional and individual isotropic B-factor refinement. NCS restraints were gradually loosened and finally fully released. The resulting model was then submitted to backbone torsion optimization followed by automated all-atom real-space refinement against a 2Fₒ-F_{c} electron density map, a method developed by Haddadian and co-workers (Haddadian et al, 2011), resulting in improved stereochemistry and electron density maps. The quality of the model was further enhanced by manual adjustments until the crystallographic R-factors R_{work} and R_{free} reached 24.0% and 26.3%, respectively, and structure quality assessed with Molprobity (Chen et al, 2010) was satisfactory. With increasing quality of the model, weak electron density became visible for the first and last few residues of the missing copy of T4L at the junctions to TM3 and TM4 in chain C, revealing that the orientation of the T4L insertion relative to its corresponding receptor was significantly different from those observed in the other two receptor-T4L fusions. Very poor or no density was, however, observed for the remaining parts of T4L. It is conceivable that due to the absence of lattice contacts in this region this portion remains disordered in a solvent-filled cavity of the crystal lattice. The final refinement statistics are presented in Table 3. Figures were prepared using PyMOL (Schrödinger).

Superposition of D₃R onto CRF₁R

D₃R (molecule A in PDB ID 3PBL) was superimposed onto CRF₁R molecule C using the Cα-atoms of the following amino-acid ranges comprising the cytoplasmic halves of TM1, TM2, TM4 and TM5 as well as entire TM3 (CRF₁R/D₃R): 130-143/43-56 (TM1), 150-162/63-75 (TM2), 193-216/108-131 (TM3), 228-234/150-156 (TM4), 282-295/203-216 (TM5). TM6 and TM7, exhibiting obvious conformational differences, were excluded.

### REFERENCES

1. Gether, U. Uncovering molecular mechanisms involved in activation of G protein-coupled receptors. Endocr. Rev. 21, 90-113 (2000).
2. Hoare, S. R. J. Mechanisms of peptide and nonpeptide ligand binding to Class B G-protein-coupled receptors. Drug discovery today 10, 417-27 (2005).
3. Perrin, M. H. & Vale, W. W. Corticotropin releasing factor receptors and their ligand family. Ann. N. Y. Acad Sci. 885, 312-28 (1999).
4. Chen, Y. L. et al. 2-aryloxy-4-alkylaminopyridines: discovery of novel corticotropin-releasing factor 1 antagonists. J. Med. Chem. 51, 1385-92 (2008).
5. Serrano-Vega, M. J., Magnani, F., Shibata, Y. & Tate, C. G. Conformational thermostabilization of the beta1-adrenergic receptor in a detergent-resistant form. Proc. Natl. Acad. Sci. USA 105, 877-82 (2008).
6. Chien, E. Y. T. et al. Structure of the human dopamine D3 receptor in complex with a D2/D3 selective antagonist. Science 330, 1091-5 (2010).
7. Ballesteros, J. A. & Weinstein, H. Integrated methods for the construction of three-dimensional models and computational probing of structure-function relations in G protein-coupled receptors. Methods Neurosci. 25, 366-428 (1995).
8. Robertson N. et al. The properties of thermostabilised G protein-coupled receptors (StaRs) and their use in drug discovery. Neuropharmacology 60, 36-44 (2011).
9. Hanson, M. A. et al. Crystal Structure of a lipid G protein-coupled receptor. Science 335, 851-855 (2012).
10. Kawate, T &Gouaux, E. Fluorescence-detection size-exclusion chromatography for precrystallization screening of integral membrane proteins. Structure 14, 673-681 (2006).
11. Caffrey, M. &Cherezov, V. Crystallizing membrane proteins using lipidic cubic mesophases. Nature Protocols 4, 706-731 (2009).
12. Kabsch, W. XDS. Actacrystallogr D 66, 125-132 (2010).
13. A. G. W. Leslie & Powell, H. R. Processing Diffraction Data with Mosflm in Read, R. J. &Sussman, J. L. (Eds.) Evolving Methods for Macromolecular Crystallography: The Structural Path to the Understanding of the Mechanism of Action of CBRN Agents pp. 41-51, 245, (2007).
14. Evans, P. R. &Murshudov, G. N., to be published (2012).
Collaborative Computational Project, Number 4. The CCP4 Suite: Programs for Protein Crystallography. ActaCryst. D 50, 760-763 (1994).
15. Matthews, B. W. Solvent content of protein crystals. J. Mol. Biol. 33, 491-497 (1968).
16. McCoy, A. J. et al. Phaser crystallographic software. J. Appl. Cryst. 40, 658-674 (2007).
17. Emsley, P., Lohkamp, B., Scott, W. G. & Cowtan, K. Features and development of Coot. ActaCryst. D 66, 486-501 (2010).
18. Adams, P. D. et al. PHENIX: a comprehensive Python-based system for macromolecular structure solution. ActaCryst. D 66, 213-221 (2010).
19. Murshudov, G. N. et al. REFMAC5 for the refinement of macromolecular crystal structures. ActaCryst. D 67, 355-367 (2011).
20. Haddadian E. J. et al. Automated real-space refinement of protein structures using a realistic backbone move set. Biophys. J. 101, 899-909 (2011).
21. Chen V. B. et al. MolProbity: all-atom structure validation for macromolecular crystallography. ActaCryst. D 66, 12-21 (2010).

### Example 2

### RMSD Calculations

We defined the global common TM region between Class A and B GPCRs as the CRF₁R residues 119-143, 150-176, 186-218, 227-247, 269-294, 312-332 and 343-365. They correspond to the Class A Ballesteros-Weinstein residues 1.35-1.59, 2.38-2.64, 3.23-3.55, 4.41-4.61, 5.40-5.65, 6.33-6.53, 7.33-7.55. For every CRF1R—Class A GPCR crystal structure the RMSDs were calculated as indicated: both molecules were initially read into Maestro and their sequences were aligned using the ‘Pairwise Alignment’ algorithm contained within the ‘Multiple Sequence Viewer’ toolbar within Maestro. Manual adjustment within the ‘Multiple Sequence Viewer’ using the ‘Grab and drag’ tool was performed to have the correct corresponding residues on the TM region (Table 4) to ensure correct alignment of corresponding residues. Residues not in the defined global common TM region were selected within the ‘Multiple Sequence Viewer’ using the ‘Select and slide’ tool. They were deleted using the ‘delete’ menu in the main window of Maestro pressing the ‘select’ tool and in the ‘Atom Selection’ pop up box pressing ‘Selection’ and ‘OK’. Protein side chains were deleted using the ‘delete’ menu in the main window of Maestro pressing the ‘select’ tool, in the ‘Atom Selection’ pop up box selecting the ‘Residue’ tab, selecting Backbone/side chain′, ticking the ‘side chain’ box and pressing ‘Add’ and ‘OK’. For the global superposition of the 7 TMs the ‘Superposition’ tool was selected from the ‘Tools’ menu in the main window of Maestro. The ‘Superimpose by ASL’ tab was selected and the ‘All’ button was pressed. The global backbone RMSD for the 7 TMs is then returned in the box at the bottom of the ‘Superposition’ tool.

Starting from this obtained structural aligned position of the 7 TMs a RMSD value for every TM was calculated. All TMs excluding the TM considered were selected within the ‘Multiple Sequence Viewer’ using the ‘Select and slide’ tool. These selected TMs not of interest were deleted using the ‘delete’ menu in the main window of Maestro pressing the ‘select’ tool and in the ‘Atom Selection’ pop up box pressing ‘Selection’ and ‘OK’. For example to analyze TM1, for all GPCRs considered TM2 to TM7 were deleted.

On the resulting individual TM the global backbone RMSD was calculated using the ‘Superposition’ tool from the ‘Tools’ menu in the main window of Maestro. The ‘Calculate in place (no transformation)’ box was ticked. The ‘Superimpose by ASL’ tab was selected and the ‘All’ button was pressed. The RMSD for the individual TM (using the starting global superimposition based on the 7 TMs) is then returned in the box at the bottom of the ‘Superposition’ tool.

Similarly starting from the individual TM obtained above the backbone RMSD was calculated after superimposition of the individual TM. The ‘Superposition’ tool from the ‘Tools’ menu in the main window of Maestro was used. The ‘Calculate in place (no transformation)’ box was not ticked. The ‘Superimpose by ASL’ tab was selected and the ‘All’ button was pressed. The RMSD for the individual TM (after superimposition of the individual TM) is then returned in the box at the bottom of the ‘Superposition’ tool.

### Tables (A)-(C)

Tables A-C show the x, y and z coordinates by amino acid residue of each non-hydrogen atom in the polypeptide structure for molecules A, B and C respectively, in addition to the antagonist CP-376395 atoms. The crystallised polypeptide is shown in FIG. 5 and is CRF1R StaR in which ICL2 is replaced with T4L.

The fourth column of the tables indicates whether the atom is from an amino acid residue of the CRF1R protein residues 115-368) (by three-letter amino acid code e.g. TRP, GLU, ALA etc), an amino acid residue of T4L (residues 1002-1161) or the CP-376395 ligand (CP3). Parameters used for the modelling are listed in the REMARK section.

POP is: 1-palmitoyl-2-oleoyl-sn-glycero-3-phospho-(1′-rac-glycerol) abbreviated as POPG; MOO is: 1-oleoyl-rac-glycerol aka monoolein.

| REMARK | Date 2012-10-04 Time 11:26:15 BST +0100 (1349346375.99 s) | | | | |
|---|---|---|---|---|---|
| REMARK | PHENIX refinement | | | | |
| REMARK | | | | | |
| REMARK | ******************** REFINEMENT SUMMARY: QUICK FACTS ******************* | | | | |
| REMARK | Start: r_work = 0.2497 r_free = 0.2616 bonds = 0.019 angles = 1.963 | | | | |
| REMARK | Final: r_work = 0.2402 r_free = 0.2630 bonds = 0.011 angles = 1.359 | | | | |
| REMARK | ************************************************************************ | | | | |
| REMARK | | | | | |
| REMARK | ****************** REFINEMENT STATISTICS STEP BY STEP ****************** | | | | |
| REMARK | leading digit, like 1_, means number of macro-cycle | | | | |
| REMARK | 0 : statistics at the very beginning when nothing is done yet | | | | |
| REMARK | 1_bss: bulk solvent correction and/or (anisotropic) scaling | | | | |
| REMARK | 1_xyz: refinement of coordinates | | | | |
| REMARK | 1_adp: refinement of ADPs (Atomic Displacement Parameters) | | | | |
| REMARK | ------------------------------------------------------------------------ | | | | |
| REMARK | R-factors, x-ray target values and norm of gradient of x-ray target | | | | |
| REMARK | stage | r-work | r-free | xray_target_w | xray_target_t |
| REMARK | 0 : 0 | 0.4038 | 0.3764 | 6.860252e+00 | 6.889859e+00 |
| REMARK | 1_bss: | 0.2497 | 0.2616 | 6.695180e+00 | 6.729690e+00 |
| REMARK | 1_ohs: | 0.2497 | 0.2616 | 6.695180e+00 | 6.729690e+00 |
| REMARK | 1_xyz: | 0.2496 | 0.2655 | 6.696200e+00 | 6.741672e+00 |
| REMARK | 1_adp: | 0.2422 | 0.2609 | 6.675479e+00 | 6.728942e+00 |
| REMARK | 2_bss: | 0.2422 | 0.2612 | 6.675391e+00 | 6.729094e+00 |
| REMARK | 2_ohs: | 0.2422 | 0.2612 | 6.675391e+00 | 6.729094e+00 |
| REMARK | 2_xyz: | 0.2409 | 0.2629 | 6.673307e+00 | 6.736986e+00 |
| REMARK | 2_adp: | 0.2402 | 0.2628 | 6.671540e+00 | 6.737154e+00 |
| REMARK | 2_bss: | 0.2402 | 0.2630 | 6.671640e+00 | 6.737068e+00 |
| REMARK | 2_ohs: | 0.2402 | 0.2630 | 6.671640e+00 | 6.737068e+00 |

| REMARK | ------------------------------------------------------------------------ | | | | |
|---|---|---|---|---|---|
| REMARK | stage | <pher> | fom | alpha | beta |
| REMARK | 0 : | 34.473 | 0.7197 | 1.2782 | 299868.375 |
| REMARK | 1_bss: | 26.650 | 0.8082 | 0.9336 | 140104.242 |
| REMARK | 1_ohs: | 26.650 | 0.8082 | 0.9336 | 140104.242 |
| REMARK | 1_xyz: | 27.068 | 0.8038 | 0.9367 | 144950.478 |
| REMARK | 1_adp: | 26.554 | 0.8093 | 0.9484 | 140726.697 |
| REMARK | 2_bss: | 26.584 | 0.8090 | 0.9282 | 140910.818 |
| REMARK | 2_ohs: | 26.584 | 0.8090 | 0.9282 | 140910.818 |
| REMARK | 2_xyz: | 26.902 | 0.8056 | 0.9258 | 143920.302 |
| REMARK | 2_adp: | 26.891 | 0.8058 | 0.9334 | 144083.992 |
| REMARK | 2_bss: | 26.888 | 0.8058 | 0.9239 | 144165.023 |
| REMARK | 2_ohs: | 26.888 | 0.8058 | 0.9239 | 144165.023 |

| REMARK | ------------------------------------------------------------------------ | | | | | | | |
|---|---|---|---|---|---|---|---|---|
| REMARK | stage | angl | bond | chir | dihe | plan | repu | geom_target |
| REMARK | 0 : | 1.963 | 0.019 | 0.132 | 13.400 | 0.020 | 4.110 | 4.3689e−01 |
| REMARK | 1_bss: | 1.963 | 0.019 | 0.132 | 13.400 | 0.020 | 4.110 | 4.3689e−01 |
| REMARK | 1_ohs: | 1.963 | 0.019 | 0.132 | 13.400 | 0.020 | 4.110 | 4.3689e−01 |
| REMARK | 1_xyz: | 1.443 | 0.012 | 0.110 | 13.283 | 0.011 | 4.113 | 1.6346e−01 |
| REMARK | 1_adp: | 1.443 | 0.012 | 0.110 | 13.283 | 0.011 | 4.113 | 1.6346e−01 |
| REMARK | 2_bss: | 1.443 | 0.012 | 0.110 | 13.283 | 0.011 | 4.113 | 1.6346e−01 |
| REMARK | 2_ohs: | 1.443 | 0.012 | 0.110 | 13.283 | 0.011 | 4.113 | 1.6346e−01 |
| REMARK | 2_xyz: | 1.359 | 0.011 | 0.100 | 12.953 | 0.010 | 4.108 | 1.5099e−01 |
| REMARK | 2_adp: | 1.359 | 0.011 | 0.100 | 12.953 | 0.010 | 4.108 | 1.5099e−01 |
| REMARK | 2_bss: | 1.359 | 0.011 | 0.100 | 12.953 | 0.010 | 4.108 | 1.5099e−01 |
| REMARK | 2_ohs: | 1.359 | 0.011 | 0.100 | 12.953 | 0.010 | 4.108 | 1.5099e−01 |

| | |
|---|---|
| REMARK | ------------------------------------------------------------------------ |

| | |
|---|---|
| REMARK | Maximal deviations: |

| | | | | | | | |
|---|---|---|---|---|---|---|---|
| REMARK | stage | angl | bond | chir dihe | plan | repu | \|grad\| |
| REMARK | 0 : | 21.739 | 0.280 | 0.878121.971 | 0.266 | 1.653 | 2.2526e−01 |
| REMARK | 1_bss: | 21.739 | 0.280 | 0.878121.971 | 0.226 | 1.653 | 2.2526e−01 |
| REMARK | 1_ohs: | 21.739 | 0.280 | 0.878121.971 | 0.226 | 1.653 | 2.2526e−01 |
| REMARK | 1_xyz: | 21.739 | 0.148 | 0.582121.971 | 0.088 | 2.064 | 9.8214e−02 |
| REMARK | 1_adp: | 21.739 | 0.148 | 0.582121.971 | 0.088 | 2.064 | 9.8214e−02 |
| REMARK | 2_bss: | 21.739 | 0.148 | 0.582121.971 | 0.088 | 2.064 | 9.8214e−02 |
| REMARK | 2_ohs: | 21.739 | 0.148 | 0.582121.971 | 0.088 | 2.064 | 9.8214e−02 |
| REMARK | 2_xyz: | 21.739 | 0.063 | 0.607121.971 | 0.078 | 2.161 | 9.6431e−02 |
| REMARK | 2_adp: | 21.739 | 0.063 | 0.607121.971 | 0.078 | 2.161 | 9.6431e−02 |
| REMARK | 2_bss: | 21.739 | 0.063 | 0.607121.971 | 0.078 | 2.161 | 9.6431e−02 |
| REMARK | 2_ohs: | 21.739 | 0.063 | 0.607121.971 | 0.078 | 2.161 | 9.6431e−02 |

| | |
|---|---|
| REMARK | ------------------------------------------------------------------------ |

| | |
|---|---|
| REMARK | \|-----overall-----\|---macromolecule----\|------solvent-------\| |

| | | | | | | | | | | |
|---|---|---|---|---|---|---|---|---|---|---|
| REMARK | stage | b_max | b_min | b_ave | b_max | b_min | b_ave | b_max | b_min | b_ave |
| REMARK | 0 : | 149.07 | 16.24 | 61.50 | 149.07 | 16.24 | 61.49 | 70.00 | 70.00 | 70.00 |
| REMARK | 1_bss: | 149.07 | 16.24 | 61.50 | 149.07 | 16.24 | 61.49 | 70.00 | 70.00 | 70.00 |
| REMARK | 1_ohs: | 149.07 | 16.24 | 61.50 | 149.07 | 16.24 | 61.49 | 70.00 | 70.00 | 70.00 |
| REMARK | 1_xyz: | 149.07 | 16.24 | 61.50 | 149.07 | 16.24 | 61.49 | 70.00 | 70.00 | 70.00 |
| REMARK | 1_adp: | 147.71 | 19.58 | 62.11 | 147.71 | 19.58 | 62.13 | 65.48 | 40.00 | 46.50 |
| REMARK | 2_bss: | 147.71 | 19.58 | 62.11 | 147.71 | 19.58 | 62.13 | 65.48 | 40.00 | 46.50 |
| REMARK | 2_ohs: | 147.71 | 19.58 | 62.11 | 147.71 | 19.58 | 62.13 | 65.48 | 40.00 | 46.50 |
| REMARK | 2_xyz: | 147.71 | 19.58 | 62.11 | 147.71 | 19.58 | 62.13 | 65.48 | 40.00 | 46.50 |
| REMARK | 2_adp: | 145.25 | 22.28 | 62.58 | 145.25 | 22.28 | 62.60 | 64.11 | 28.15 | 44.42 |
| REMARK | 2_bss: | 145.25 | 22.28 | 62.58 | 145.25 | 22.28 | 62.60 | 64.11 | 28.15 | 44.42 |
| REMARK | 2_ohs: | 145.25 | 22.28 | 62.58 | 145.25 | 22.28 | 62.60 | 64.11 | 28.15 | 44.42 |

| REMARK | ------------------------------------------------------------------------ | | | |
|---|---|---|---|---|
| REMARK | stage | Deviation of refined | | |
| REMARK | | model from start model | | |
| REMARK | | max | min | mean |
| REMARK | 0 : | 0.000 | 0.000 | 0.000 |
| REMARK | 1_bss: | 0.000 | 0.000 | 0.000 |
| REMARK | 1_ohs: | 0.000 | 0.000 | 0.000 |
| REMARK | 1_xyz: | 2.547 | 0.000 | 0.047 |
| REMARK | 1_adp: | 2.547 | 0.000 | 0.047 |
| REMARK | 2_bss: | 2.547 | 0.000 | 0.047 |
| REMARK | 2_ohs: | 2.547 | 0.000 | 0.047 |
| REMARK | 2_xyz: | 2.594 | 0.000 | 0.068 |
| REMARK | 2_adp: | 2.594 | 0.000 | 0.068 |
| REMARK | 2_bss: | 2.594 | 0.000 | 0.068 |
| REMARK | 2_ohs: | 2.594 | 0.000 | 0.068 |

| REMARK | ------------------------------------------------------------------------ | | |
|---|---|---|---|
| REMARK | MODEL CONTENT. | | |
| REMARK | ELEMENT | ATOM RECORD COUNT | OCCUPANCY SUM |
| REMARK | P | 4 | 4.00 |
| REMARK | C | 5902 | 5902.00 |
| REMARK | S | 52 | 52.00 |
| REMARK | O | 1509 | 1509.00 |
| REMARK | N | 1445 | 1445.00 |
| REMARK | TOTAL | 8912 | 8912.00 |

| REMARK | ----------------------------------------------------------------------- |
|---|---|
| REMARK | r_free_flags.md5.hexdigest b100cf5a715fdf937066e78b8999dcd3 |

REMARK

| REMARK | IF THIS FILE IS FOR PDB DEPOSITION: REMOVE ALL FROM THIS LINE UP. | |
|---|---|---|
| REMARK | 3 | |
| REMARK | 3 | REFINEMENT. |

| | | |
|---|---|---|
| REMARK | 3 | PROGRAM : PHENIX (phenix.refine: 1.8.1_1168) |
| REMARK | 3 | AUTHORS : Adams,Afonine,Chen,Davis,Echols,Gildea,Gopal, |

| | | |
|---|---|---|
| REMARK | 3 | : Grosse-Kunstleve,Headd,Hung,Immormino,Ioerger,McCoy, |
| REMARK | 3 | : McKee,Moriarty,Pai,Read,Richardson,Richardson,Romo, |
| REMARK | 3 | : Sacchettini,Sauter,Smith,Storoni,Terwilliger,Zwart |

| REMARK | 3 | | |
|---|---|---|---|
| REMARK | 3 | REFINEMENT TARGET | : ML |
| REMARK | 3 | | |

| | | |
|---|---|---|
| REMARK | 3 | DATA USED IN REFINEMENT. |

| | | | |
|---|---|---|---|
| REMARK | 3 | RESOLUTION RANGE HIGH (ANGSTROMS) | : 2.977 |
| REMARK | 3 | RESOLUTION RANGE LOW (ANGSTROMS) | : 34.155 |
| REMARK | 3 | MIN(FOBS/SIGMA_FOBS) | : 0.89 |
| REMARK | 3 | COMPLETENESS FOR RANGE (%) | : 67.01 |
| REMARK | 3 | NUMBER OF REFLECTIONS | : 47564 |
| REMARK | 3 | NUMBER OF REFLECTIONS (NON-ANOMALOUS) | : 32114 |

| REMARK | 3 | |
|---|---|---|
| REMARK | 3 | FIT TO DATA USED IN REFINEMENT. |

| | | | |
|---|---|---|---|
| REMARK | 3 | R VALUE (WORKING + TEST SET) | : 0.2413 |
| REMARK | 3 | R VALUE (WORKING SET) | : 0.2402 |
| REMARK | 3 | FREE R VALUE | : 0.2630 |
| REMARK | 3 | FREE R VALUE TEST SET SIZE (%) | : 4.98 |
| REMARK | 3 | FREE R VALUE TEST SET COUNT | : 2371 |

| REMARK | 3 | | | | | | | |
|---|---|---|---|---|---|---|---|---|
| REMARK | 3 | FIT TO DATA USED IN REFINEMENT (IN BINS). | | | | | | |
| REMARK | 3 | BIN | RESOLUTION RANGE | COMPL. | NWORK | NFREE | RWORK | RFREE |
| REMARK | 3 | 1 | 34.1572 - 7.6288 | 0.78 | 3089 | 172 | 0.2256 | 0.2141 |
| REMARK | 3 | 2 | 7.6288 - 6.0662 | 0.76 | 3025 | 171 | 0.2331 | 0.2423 |
| REMARK | 3 | 3 | 6.0662 - 5.3026 | 0.74 | 2939 | 154 | 0.2341 | 0.3081 |
| REMARK | 3 | 4 | 5.3026 - 4.8193 | 0.76 | 3006 | 157 | 0.2072 | 0.2135 |
| REMARK | 3 | 5 | 4.8193 - 4.4747 | 0.77 | 3066 | 161 | 0.1939 | 0.2018 |
| REMARK | 3 | 6 | 4.4747 - 4.2113 | 0.74 | 2930 | 150 | 0.2159 | 0.2709 |
| REMARK | 3 | 7 | 4.2113 - 4.0008 | 0.72 | 2854 | 143 | 0.2194 | 0.2629 |
| REMARK | 3 | 8 | 4.0008 - 3.8269 | 0.72 | 2890 | 153 | 0.2335 | 0.2666 |
| REMARK | 3 | 9 | 3.8269 - 3.6797 | 0.70 | 2736 | 163 | 0.2396 | 0.2586 |
| REMARK | 3 | 10 | 3.6797 - 3.5529 | 0.69 | 2747 | 131 | 0.2479 | 0.2833 |
| REMARK | 3 | 11 | 3.5529 - 3.4419 | 0.64 | 2573 | 109 | 0.2660 | 0.3080 |
| REMARK | 3 | 12 | 3.4419 - 3.3436 | 0.65 | 2586 | 146 | 0.2799 | 0.3004 |
| REMARK | 3 | 13 | 3.3436 - 3.2556 | 0.62 | 2437 | 148 | 0.2888 | 0.3017 |
| REMARK | 3 | 14 | 3.2556 - 3.1763 | 0.59 | 2360 | 84 | 0.3116 | 0.3476 |
| REMARK | 3 | 15 | 3.1763 - 3.1041 | 0.58 | 2329 | 125 | 0.3242 | 0.4090 |
| REMARK | 3 | 16 | 3.1041 - 3.0381 | 0.57 | 2268 | 130 | 0.3461 | 0.3801 |
| REMARK | 3 | 17 | 3.0381 - 2.9774 | 0.34 | 1358 | 74 | 0.3492 | 0.3086 |
| REMARK | 3 | | | | | | | |

| | | |
|---|---|---|
| REMARK | 3 | BULK SOLVENT MODELLING. |

| | | |
|---|---|---|
| REMARK | 3 | METHOD USED : FLAT BULK SOLVENT MODEL |
| REMARK | 3 | SOLVENT RADIUS : 1.11 |
| REMARK | 3 | SHRINKAGE RADIUS : 0.90 |
| REMARK | 3 | GRID STEP FACTOR : 4.00 |

| | | |
|---|---|---|
| REMARK | 3 | |
| REMARK | 3 | ERROR ESTIMATES. |

| | | |
|---|---|---|
| REMARK | 3 | COORDINATE ERROR (MAXIMUM-LIKELIHOOD BASED) : 0.43 |
| REMARK | 3 | PHASE ERROR (DEGREES, MAXIMUM-LIKELIHOOD BASED) : 26.89 |

| REMARK | 3 | | | | |
|---|---|---|---|---|---|
| REMARK | 3 | STRUCTURE FACTORS CALCULATION ALGORITHM : FFT | | | |
| REMARK | 3 | | | | |
| REMARK | 3 | DEVIATIONS FROM IDEAL VALUES. | | | |
| REMARK | 3 | | RMSD | MAX | NT |
| REMARK | 3 | BOND | : 0.011 | 0.063 | 9105 |
| REMARK | 3 | ANGLE | : 1.359 | 21.739 | 12279 |
| REMARK | 3 | CHIRAL | : 0.100 | 0.607 | 1347 |
| REMARK | 3 | PLANARITY | : 0.010 | 0.078 | 1469 |
| REMARK | 3 | DIHEDRAL | : 12.953 | 121.971 | 3393 |

| | | | |
|---|---|---|---|
| REMARK | 3 | MIN NONBONDED DISTANCE | : 2.161 |

| REMARK | 3 | |
|---|---|---|
| REMARK | 3 | MOLPROBITY STATISTICS. |

| | | | |
|---|---|---|---|
| REMARK | 3 | ALL-ATOM CLASHSCORE | : 9.63 |

| | | |
|---|---|---|
| REMARK | 3 | RAMACHANDRAN PLOT: |

| | | | |
|---|---|---|---|
| REMARK | 3 | OUTLIERS | : 0.00 % |
| REMARK | 3 | ALLOWED | : 1.94 % |
| REMARK | 3 | FAVORED | : 98.06 % |

| | | | |
|---|---|---|---|
| REMARK | 3 | ROTAMER OUTLIERS | : 0.79 % |
| REMARK | 3 | CBETA DEVIATIONS | : 0 |

| REMARK | 3 | |
|---|---|---|
| REMARK | 3 | ATOMIC DISPLACEMENT PARAMETERS. |

| | | | |
|---|---|---|---|
| REMARK | 3 | WILSON B | : 54.40 |
| REMARK | 3 | RMS(B_ISO_OR_EQUIVALENT_BONDED) | : 5.41 |

| REMARK | 3 | ATOMS | NUMBER OF ATOMS | |
|---|---|---|---|---|
| REMARK | 3 | | ISO. | ANISO. |
| REMARK | 3 | ALL | : 8912 | 0 |
| REMARK | 3 | ALL (NO H) | : 8912 | 0 |
| REMARK | 3 | SOLVENT | : 14 | 0 |
| REMARK | 3 | NONON-SOLVENT | : 8898 | 0 |
| REMARK | 3 | HYDROGENS | : 0 | 0 |
| REMARK | 3 | | | |

| | | | | | | | | | |
|---|---|---|---|---|---|---|---|---|---|
| CRYST1 | 86.559 | 123.968 | 166.834 | 90.00 | 90.00 | 90.00 | P 2 | 21 | 21 |

| | | | | |
|---|---|---|---|---|
| SCALE1 | 0.011553 | 0.000000 | 0.000000 | 0.00000 |
| SCALE2 | 0.000000 | 0.008067 | 0.000000 | 0.00000 |
| SCALE3 | 0.000000 | 0.000000 | 0.005994 | 0.00000 |

TABLE A

| | | | | | | | | | | | |
|---|---|---|---|---|---|---|---|---|---|---|---|
| ATOM | 1 | N | HIS | A | 115 | −68.942 | −28.570 | 48.217 | 1.00 | 98.25 | N |
| ATOM | 2 | CA | HIS | A | 115 | −67.468 | −28.576 | 48.076 | 1.00 | 100.71 | C |
| ATOM | 3 | CB | HIS | A | 115 | −67.018 | −29.565 | 47.006 | 1.00 | 101.77 | C |
| ATOM | 4 | CG | HIS | A | 115 | −67.457 | −30.976 | 47.246 | 1.00 | 110.71 | C |
| ATOM | 5 | ND1 | HIS | A | 115 | −66.839 | −31.807 | 48.155 | 1.00 | 117.28 | N |
| ATOM | 6 | CE1 | HIS | A | 115 | −67.428 | −32.990 | 48.134 | 1.00 | 114.02 | C |
| ATOM | 7 | NE2 | HIS | A | 115 | −68.395 | −32.959 | 47.237 | 1.00 | 110.39 | N |
| ATOM | 8 | CD2 | HIS | A | 115 | −68.434 | −31.710 | 46.664 | 1.00 | 110.87 | C |
| ATOM | 9 | C | HIS | A | 115 | −66.919 | −27.198 | 47.718 | 1.00 | 96.55 | C |
| ATOM | 10 | O | HIS | A | 115 | −65.772 | −26.894 | 47.983 | 1.00 | 94.86 | O |
| ATOM | 11 | N | TYR | A | 116 | −67.721 | −26.409 | 47.000 | 1.00 | 107.80 | N |
| ATOM | 12 | CA | TYR | A | 116 | −67.260 | −25.150 | 46.414 | 1.00 | 104.15 | C |
| ATOM | 13 | CB | TYR | A | 116 | −68.391 | −24.444 | 45.661 | 1.00 | 116.51 | C |
| ATOM | 14 | CG | TYR | A | 116 | −69.020 | −25.222 | 44.529 | 1.00 | 127.07 | C |
| ATOM | 15 | CD1 | TYR | A | 116 | −68.365 | −26.294 | 43.934 | 1.00 | 120.18 | C |
| ATOM | 16 | CE1 | TYR | A | 116 | −68.950 | −26.996 | 42.897 | 1.00 | 128.52 | C |
| ATOM | 17 | CZ | TYR | A | 116 | −70.200 | −26.625 | 42.440 | 1.00 | 137.23 | C |
| ATOM | 18 | OH | TYR | A | 116 | −70.793 | −27.315 | 41.408 | 1.00 | 125.67 | O |
| ATOM | 19 | CE2 | TYR | A | 116 | −70.866 | −25.563 | 43.012 | 1.00 | 134.05 | C |
| ATOM | 20 | CD2 | TYR | A | 116 | −70.276 | −24.869 | 44.046 | 1.00 | 132.85 | C |
| ATOM | 21 | C | TYR | A | 116 | −66.777 | −24.199 | 47.530 | 1.00 | 98.50 | C |
| ATOM | 22 | O | TYR | A | 116 | −65.717 | −23.567 | 47.430 | 1.00 | 100.40 | O |
| ATOM | 23 | N | HIS | A | 117 | −67.553 | −24.181 | 48.622 | 1.00 | 95.43 | N |
| ATOM | 24 | CA | HIS | A | 117 | −67.205 | −23.488 | 49.850 | 1.00 | 85.02 | C |
| ATOM | 25 | CB | HIS | A | 117 | −68.333 | −23.602 | 50.878 | 1.00 | 89.78 | C |
| ATOM | 26 | CG | HIS | A | 117 | −69.644 | −23.062 | 50.392 | 1.00 | 102.34 | C |
| ATOM | 27 | ND1 | HIS | A | 117 | −69.875 | −21.714 | 50.220 | 1.00 | 102.31 | N |
| ATOM | 28 | CE1 | HIS | A | 117 | −71.108 | −21.535 | 49.780 | 1.00 | 95.09 | C |
| ATOM | 29 | NE2 | HIS | A | 117 | −71.684 | −22.716 | 49.660 | 1.00 | 99.53 | N |
| ATOM | 30 | CD2 | HIS | A | 117 | −70.789 | −23.690 | 50.035 | 1.00 | 104.83 | C |
| ATOM | 31 | C | HIS | A | 117 | −65.880 | −23.987 | 50.435 | 1.00 | 79.77 | C |
| ATOM | 32 | O | HIS | A | 117 | −65.050 | −23.201 | 50.864 | 1.00 | 79.95 | O |
| ATOM | 33 | N | VAL | A | 118 | −65.662 | −25.301 | 50.397 | 1.00 | 79.10 | N |
| ATOM | 34 | CA | VAL | A | 118 | −64.380 | −25.880 | 50.817 | 1.00 | 81.08 | C |
| ATOM | 35 | CB | VAL | A | 118 | −64.387 | −27.421 | 50.787 | 1.00 | 80.49 | C |
| ATOM | 36 | CG1 | VAL | A | 118 | −63.038 | −27.967 | 51.237 | 1.00 | 73.07 | C |
| ATOM | 37 | CG2 | VAL | A | 118 | −65.500 | −27.959 | 51.671 | 1.00 | 81.76 | C |
| ATOM | 38 | C | VAL | A | 118 | −63.206 | −25.335 | 49.989 | 1.00 | 75.57 | C |
| ATOM | 39 | O | VAL | A | 118 | −62.211 | −24.884 | 50.522 | 1.00 | 67.15 | O |
| ATOM | 40 | N | ALA | A | 119 | −63.372 | −25.319 | 48.667 | 1.00 | 77.14 | N |
| ATOM | 41 | CA | ALA | A | 119 | −62.337 | −24.823 | 47.765 | 1.00 | 74.53 | C |
| ATOM | 42 | CB | ALA | A | 119 | −62.730 | −25.068 | 46.318 | 1.00 | 82.48 | C |
| ATOM | 43 | C | ALA | A | 119 | −62.030 | −23.331 | 47.995 | 1.00 | 66.21 | C |
| ATOM | 44 | O | ALA | A | 119 | −60.880 | −22.929 | 48.011 | 1.00 | 71.56 | O |
| ATOM | 45 | N | ALA | A | 120 | −63.073 | −22.536 | 48.236 | 1.00 | 60.73 | N |
| ATOM | 46 | CA | ALA | A | 120 | −62.949 | −21.128 | 48.621 | 1.00 | 58.74 | C |
| ATOM | 47 | CB | ALA | A | 120 | −64.325 | −20.485 | 48.721 | 1.00 | 64.51 | C |
| ATOM | 48 | C | ALA | A | 120 | −62.169 | −20.910 | 49.922 | 1.00 | 56.57 | C |
| ATOM | 49 | O | ALA | A | 120 | −61.313 | −20.051 | 49.996 | 1.00 | 51.84 | O |
| ATOM | 50 | N | ILE | A | 121 | −62.493 | −21.718 | 50.941 | 1.00 | 65.53 | N |
| ATOM | 51 | CA | ILE | A | 121 | −61.839 | −21.675 | 52.273 | 1.00 | 63.26 | C |
| ATOM | 52 | CB | ILE | A | 121 | −62.512 | −22.658 | 53.258 | 1.00 | 56.49 | C |
| ATOM | 53 | CG1 | ILE | A | 121 | −63.899 | −22.147 | 53.648 | 1.00 | 60.52 | C |
| ATOM | 54 | CD1 | ILE | A | 121 | −64.685 | −23.119 | 54.494 | 1.00 | 69.66 | C |
| ATOM | 55 | CG2 | ILE | A | 121 | −61.662 | −22.850 | 54.503 | 1.00 | 40.62 | C |
| ATOM | 56 | C | ILE | A | 121 | −60.357 | −21.985 | 52.188 | 1.00 | 53.08 | C |
| ATOM | 57 | O | ILE | A | 121 | −59.535 | −21.289 | 52.747 | 1.00 | 48.00 | O |
| ATOM | 58 | N | ILE | A | 122 | −60.043 | −23.023 | 51.410 | 1.00 | 61.39 | N |
| ATOM | 59 | CA | ILE | A | 122 | −58.673 | −23.410 | 51.111 | 1.00 | 62.10 | C |
| ATOM | 60 | CB | ILE | A | 122 | −58.618 | −24.694 | 50.256 | 1.00 | 60.64 | C |
| ATOM | 61 | CG1 | ILE | A | 122 | −59.252 | −25.865 | 51.005 | 1.00 | 61.81 | C |
| ATOM | 62 | CD1 | ILE | A | 122 | −59.268 | −27.153 | 50.211 | 1.00 | 71.04 | C |
| ATOM | 63 | CG2 | ILE | A | 122 | −57.183 | −25.032 | 49.880 | 1.00 | 54.34 | C |
| ATOM | 64 | C | ILE | A | 122 | −57.938 | −22.297 | 50.369 | 1.00 | 61.26 | C |
| ATOM | 65 | O | ILE | A | 122 | −56.806 | −21.991 | 50.680 | 1.00 | 58.16 | O |
| ATOM | 66 | N | ASN | A | 123 | −58.600 | −21.693 | 49.372 | 1.00 | 62.92 | N |
| ATOM | 67 | CA | ASN | A | 123 | −58.003 | −20.566 | 48.660 | 1.00 | 57.82 | C |
| ATOM | 68 | CB | ASN | A | 123 | −58.888 | −20.122 | 47.491 | 1.00 | 57.77 | C |
| ATOM | 69 | CG | ASN | A | 123 | −58.435 | −20.701 | 46.163 | 1.00 | 61.99 | C |
| ATOM | 70 | OD1 | ASN | A | 123 | −57.273 | −21.071 | 45.997 | 1.00 | 63.07 | O |
| ATOM | 71 | ND2 | ASN | A | 123 | −59.351 | −20.772 | 45.206 | 1.00 | 67.41 | N |
| ATOM | 72 | C | ASN | A | 123 | −57.672 | −19.360 | 49.557 | 1.00 | 56.02 | C |
| ATOM | 73 | O | ASN | A | 123 | −56.584 | −18.824 | 49.514 | 1.00 | 61.20 | O |
| ATOM | 74 | N | TYR | A | 124 | −58.584 | −18.956 | 50.426 | 1.00 | 53.74 | N |
| ATOM | 75 | CA | TYR | A | 124 | −58.313 | −17.853 | 51.323 | 1.00 | 50.36 | C |
| ATOM | 76 | CB | TYR | A | 124 | −59.592 | −17.426 | 52.062 | 1.00 | 40.38 | C |
| ATOM | 77 | CG | TYR | A | 124 | −59.360 | −16.380 | 53.139 | 1.00 | 45.50 | C |
| ATOM | 78 | CD1 | TYR | A | 124 | −59.425 | −15.020 | 52.852 | 1.00 | 41.65 | C |
| ATOM | 79 | CE1 | TYR | A | 124 | −59.199 | −14.069 | 53.840 | 1.00 | 37.43 | C |
| ATOM | 80 | CZ | TYR | A | 124 | −58.905 | −14.479 | 55.125 | 1.00 | 41.59 | C |
| ATOM | 81 | OH | TYR | A | 124 | −58.676 | −13.554 | 56.120 | 1.00 | 47.01 | O |
| ATOM | 82 | CE2 | TYR | A | 124 | −58.838 | −15.820 | 55.429 | 1.00 | 39.69 | C |
| ATOM | 83 | CD2 | TYR | A | 124 | −59.065 | −16.758 | 54.444 | 1.00 | 43.28 | C |
| ATOM | 84 | C | TYR | A | 124 | −57.207 | −18.219 | 52.310 | 1.00 | 45.69 | C |
| ATOM | 85 | O | TYR | A | 124 | −56.365 | −17.416 | 52.662 | 1.00 | 38.86 | O |
| ATOM | 86 | N | LEU | A | 125 | −57.259 | −19.443 | 52.804 | 1.00 | 49.48 | N |
| ATOM | 87 | CA | LEU | A | 125 | −56.259 | −19.929 | 53.733 | 1.00 | 49.35 | C |
| ATOM | 88 | CB | LEU | A | 125 | −56.629 | −21.340 | 54.190 | 1.00 | 46.51 | C |
| ATOM | 89 | CG | LEU | A | 125 | −55.997 | −21.836 | 55.487 | 1.00 | 48.00 | C |
| ATOM | 90 | CD1 | LEU | A | 125 | −56.743 | −21.271 | 56.689 | 1.00 | 53.04 | C |
| ATOM | 91 | CD2 | LEU | A | 125 | −55.973 | −23.355 | 55.519 | 1.00 | 52.93 | C |
| ATOM | 92 | C | LEU | A | 125 | −54.854 | −19.949 | 53.136 | 1.00 | 53.54 | C |
| ATOM | 93 | O | LEU | A | 125 | −53.896 | −19.462 | 53.705 | 1.00 | 58.99 | O |
| ATOM | 94 | N | GLY | A | 126 | −54.756 | −20.474 | 51.941 | 1.00 | 48.30 | N |
| ATOM | 95 | CA | GLY | A | 126 | −53.529 | −20.526 | 51.224 | 1.00 | 54.36 | C |
| ATOM | 96 | C | GLY | A | 126 | −52.952 | −19.213 | 50.895 | 1.00 | 55.80 | C |
| ATOM | 97 | O | GLY | A | 126 | −51.780 | −19.001 | 51.048 | 1.00 | 54.52 | O |
| ATOM | 98 | N | HIS | A | 127 | −53.777 | −18.323 | 50.400 | 1.00 | 51.38 | N |
| ATOM | 99 | CA | HIS | A | 127 | −53.361 | −16.999 | 50.029 | 1.00 | 47.48 | C |
| ATOM | 100 | CB | HIS | A | 127 | −54.472 | −16.226 | 49.325 | 1.00 | 46.43 | C |
| ATOM | 101 | CG | HIS | A | 127 | −54.547 | −16.518 | 47.858 | 1.00 | 50.17 | C |
| ATOM | 102 | ND1 | HIS | A | 127 | −55.120 | −17.663 | 47.359 | 1.00 | 52.31 | N |
| ATOM | 103 | CE1 | HIS | A | 127 | −55.020 | −17.661 | 46.038 | 1.00 | 50.31 | C |
| ATOM | 104 | NE2 | HIS | A | 127 | −54.392 | −16.563 | 45.671 | 1.00 | 48.29 | N |
| ATOM | 105 | CD2 | HIS | A | 127 | −54.074 | −15.830 | 46.792 | 1.00 | 50.02 | C |
| ATOM | 106 | C | HIS | A | 127 | −52.804 | −16.249 | 51.229 | 1.00 | 44.51 | C |
| ATOM | 107 | O | HIS | A | 127 | −51.777 | −15.612 | 51.140 | 1.00 | 52.67 | O |
| ATOM | 108 | N | CYS | A | 128 | −53.448 | −16.412 | 52.381 | 1.00 | 42.50 | N |
| ATOM | 109 | CA | CYS | A | 128 | −52.938 | −15.870 | 53.622 | 1.00 | 36.96 | C |
| ATOM | 110 | CB | CYS | A | 128 | −53.903 | −16.144 | 54.777 | 1.00 | 35.91 | C |
| ATOM | 111 | SG | CYS | A | 128 | −55.429 | −15.173 | 54.717 | 1.00 | 51.88 | S |
| ATOM | 112 | C | CYS | A | 128 | −51.568 | −16.412 | 53.953 | 1.00 | 45.89 | C |
| ATOM | 113 | O | CYS | A | 128 | −50.664 | −15.663 | 54.241 | 1.00 | 45.89 | O |
| ATOM | 114 | N | ILE | A | 129 | −51.433 | −17.735 | 53.925 | 1.00 | 47.97 | N |
| ATOM | 115 | CA | ILE | A | 129 | −50.157 | −18.390 | 54.162 | 1.00 | 46.14 | C |
| ATOM | 116 | CB | ILE | A | 129 | −50.292 | −19.923 | 54.184 | 1.00 | 51.00 | C |
| ATOM | 117 | CG1 | ILE | A | 129 | −51.072 | −20.352 | 55.427 | 1.00 | 42.76 | C |
| ATOM | 118 | CD1 | ILE | A | 129 | −51.354 | −21.835 | 55.482 | 1.00 | 47.11 | C |
| ATOM | 119 | CG2 | ILE | A | 129 | −48.919 | −20.585 | 54.169 | 1.00 | 43.54 | C |
| ATOM | 120 | C | ILE | A | 129 | −49.074 | −17.973 | 53.205 | 1.00 | 45.71 | C |
| ATOM | 121 | O | ILE | A | 129 | −48.050 | −17.486 | 53.652 | 1.00 | 41.86 | O |
| ATOM | 122 | N | SER | A | 130 | −49.300 | −18.092 | 51.875 | 1.00 | 46.30 | N |
| ATOM | 123 | CA | SER | A | 130 | −48.324 | −17.598 | 50.963 | 1.00 | 48.10 | C |
| ATOM | 124 | CB | SER | A | 130 | −48.797 | −17.818 | 49.528 | 1.00 | 51.10 | C |
| ATOM | 125 | OG | SER | A | 130 | −48.919 | −19.197 | 49.255 | 1.00 | 52.81 | O |
| ATOM | 126 | C | SER | A | 130 | −47.961 | −16.128 | 51.149 | 1.00 | 43.60 | C |
| ATOM | 127 | O | SER | A | 130 | −46.806 | −15.792 | 51.109 | 1.00 | 44.60 | O |
| ATOM | 128 | N | LEU | A | 131 | −48.926 | −15.249 | 51.359 | 1.00 | 44.61 | N |
| ATOM | 129 | CA | LEU | A | 131 | −48.618 | −13.832 | 51.579 | 1.00 | 44.05 | C |
| ATOM | 130 | CB | LEU | A | 131 | −49.900 | −13.007 | 51.739 | 1.00 | 42.42 | C |
| ATOM | 131 | CG | LEU | A | 131 | −49.698 | −11.505 | 51.964 | 1.00 | 41.06 | C |
| ATOM | 132 | CD1 | LEU | A | 131 | −48.794 | −10.919 | 50.892 | 1.00 | 39.87 | C |
| ATOM | 133 | CD2 | LEU | A | 131 | −51.034 | −10.783 | 51.982 | 1.00 | 38.64 | C |
| ATOM | 134 | C | LEU | A | 131 | −47.682 | −13.575 | 52.745 | 1.00 | 40.78 | C |
| ATOM | 135 | O | LEU | A | 131 | −46.742 | −12.843 | 52.629 | 1.00 | 38.14 | O |
| ATOM | 136 | N | VAL | A | 132 | −47.958 | −14.178 | 53.879 | 1.00 | 42.02 | N |
| ATOM | 137 | CA | VAL | A | 132 | −47.090 | −14.089 | 55.037 | 1.00 | 40.56 | C |
| ATOM | 138 | CB | VAL | A | 132 | −47.717 | −14.778 | 56.278 | 1.00 | 36.33 | C |
| ATOM | 139 | CG1 | VAL | A | 132 | −46.704 | −14.913 | 57.405 | 1.00 | 39.47 | C |
| ATOM | 140 | CG2 | VAL | A | 132 | −48.917 | −13.993 | 56.756 | 1.00 | 34.89 | C |
| ATOM | 141 | C | VAL | A | 132 | −45.697 | −14.636 | 54.761 | 1.00 | 43.11 | C |
| ATOM | 142 | O | VAL | A | 132 | −44.708 | −14.044 | 55.115 | 1.00 | 45.24 | O |
| ATOM | 143 | N | ALA | A | 133 | −45.633 | −15.779 | 54.101 | 1.00 | 41.91 | N |
| ATOM | 144 | CA | ALA | A | 133 | −44.375 | −16.409 | 53.734 | 1.00 | 42.61 | C |
| ATOM | 145 | CB | ALA | A | 133 | −44.634 | −17.753 | 53.061 | 1.00 | 41.12 | C |
| ATOM | 146 | C | ALA | A | 133 | −43.486 | −15.537 | 52.859 | 1.00 | 46.63 | C |
| ATOM | 147 | O | ALA | A | 133 | −42.325 | −15.334 | 53.143 | 1.00 | 45.69 | O |
| ATOM | 148 | N | LEU | A | 134 | −44.098 | −14.964 | 51.827 | 1.00 | 45.81 | N |
| ATOM | 149 | CA | LEU | A | 134 | −43.457 | −14.019 | 50.921 | 1.00 | 41.26 | C |
| ATOM | 150 | CB | LEU | A | 134 | −44.440 | −13.577 | 49.836 | 1.00 | 36.02 | C |
| ATOM | 151 | CG | LEU | A | 134 | −44.825 | −14.640 | 48.809 | 1.00 | 40.53 | C |
| ATOM | 152 | CD1 | LEU | A | 134 | −46.015 | −14.186 | 47.973 | 1.00 | 38.83 | C |
| ATOM | 153 | CD2 | LEU | A | 134 | −43.635 | −14.965 | 47.922 | 1.00 | 39.42 | C |
| ATOM | 154 | C | LEU | A | 134 | −42.912 | −12.807 | 51.657 | 1.00 | 41.92 | C |
| ATOM | 155 | O | LEU | A | 134 | −41.787 | −12.432 | 51.493 | 1.00 | 40.44 | O |
| ATOM | 156 | N | LEU | A | 135 | −43.713 | −12.235 | 52.519 | 1.00 | 42.63 | N |
| ATOM | 157 | CA | LEU | A | 135 | −43.334 | −11.077 | 53.291 | 1.00 | 39.70 | C |
| ATOM | 158 | CB | LEU | A | 135 | −44.519 | −10.585 | 54.127 | 1.00 | 35.89 | C |
| ATOM | 159 | CG | LEU | A | 135 | −45.604 | −9.811 | 53.378 | 1.00 | 40.51 | C |
| ATOM | 160 | CD1 | LEU | A | 135 | −46.923 | −9.854 | 54.129 | 1.00 | 36.61 | C |
| ATOM | 161 | CD2 | LEU | A | 135 | −45.165 | −8.374 | 53.160 | 1.00 | 42.05 | C |
| ATOM | 162 | C | LEU | A | 135 | −42.160 | −11.386 | 54.208 | 1.00 | 44.85 | C |
| ATOM | 163 | O | LEU | A | 135 | −41.268 | −10.589 | 54.376 | 1.00 | 41.20 | O |
| ATOM | 164 | N | VAL | A | 136 | −42.154 | −12.566 | 54.799 | 1.00 | 47.50 | N |
| ATOM | 165 | CA | VAL | A | 136 | −41.042 | −12.974 | 55.650 | 1.00 | 48.33 | C |
| ATOM | 166 | CB | VAL | A | 136 | −41.356 | −14.303 | 56.386 | 1.00 | 44.41 | C |
| ATOM | 167 | CG1 | VAL | A | 136 | −40.081 | −14.964 | 56.904 | 1.00 | 43.56 | C |
| ATOM | 168 | CG2 | VAL | A | 136 | −42.339 | −14.059 | 57.520 | 1.00 | 39.11 | C |
| ATOM | 169 | C | VAL | A | 136 | −39.761 | −13.132 | 54.844 | 1.00 | 51.77 | C |
| ATOM | 170 | O | VAL | A | 136 | −38.707 | −12.691 | 55.256 | 1.00 | 53.00 | O |
| ATOM | 171 | N | ALA | A | 137 | −39.880 | −13.725 | 53.657 | 1.00 | 52.80 | N |
| ATOM | 172 | CA | ALA | A | 137 | −38.748 | −13.911 | 52.762 | 1.00 | 43.98 | C |
| ATOM | 173 | CB | ALA | A | 137 | −39.134 | −14.796 | 51.583 | 1.00 | 33.70 | C |
| ATOM | 174 | C | ALA | A | 137 | −38.168 | −12.592 | 52.279 | 1.00 | 42.93 | C |
| ATOM | 175 | O | ALA | A | 137 | −36.991 | −12.418 | 52.188 | 1.00 | 44.16 | O |
| ATOM | 176 | N | PHE | A | 138 | −39.035 | −11.655 | 51.986 | 1.00 | 45.70 | N |
| ATOM | 177 | CA | PHE | A | 138 | −38.646 | −10.338 | 51.536 | 1.00 | 46.36 | C |
| ATOM | 178 | CB | PHE | A | 138 | −39.924 | −9.569 | 51.212 | 1.00 | 44.14 | C |
| ATOM | 179 | CG | PHE | A | 138 | −39.703 | −8.245 | 50.561 | 1.00 | 38.32 | C |
| ATOM | 180 | CD1 | PHE | A | 138 | −39.405 | −8.166 | 49.215 | 1.00 | 43.50 | C |
| ATOM | 181 | CE1 | PHE | A | 138 | −39.229 | −6.944 | 48.603 | 1.00 | 50.75 | C |
| ATOM | 182 | CZ | PHE | A | 138 | −39.364 | −5.782 | 49.333 | 1.00 | 56.68 | C |
| ATOM | 183 | CE2 | PHE | A | 138 | −39.676 | −5.848 | 50.675 | 1.00 | 51.85 | C |
| ATOM | 184 | CD2 | PHE | A | 138 | −39.848 | −7.075 | 51.282 | 1.00 | 42.99 | C |
| ATOM | 185 | C | PHE | A | 138 | −37.826 | −9.585 | 52.580 | 1.00 | 48.89 | C |
| ATOM | 186 | O | PHE | A | 138 | −36.785 | −9.047 | 52.287 | 1.00 | 54.24 | O |
| ATOM | 187 | N | VAL | A | 139 | −38.294 | −9.592 | 53.823 | 1.00 | 49.28 | N |
| ATOM | 188 | CA | VAL | A | 139 | −37.593 | −8.962 | 54.920 | 1.00 | 45.52 | C |
| ATOM | 189 | CB | VAL | A | 139 | −38.403 | −9.019 | 56.230 | 1.00 | 32.98 | C |
| ATOM | 190 | CG1 | VAL | A | 139 | −37.594 | −8.455 | 57.381 | 1.00 | 33.63 | C |
| ATOM | 191 | CG2 | VAL | A | 139 | −39.692 | −8.248 | 56.072 | 1.00 | 32.41 | C |
| ATOM | 192 | C | VAL | A | 139 | −36.247 | −9.601 | 55.124 | 1.00 | 43.73 | C |
| ATOM | 193 | O | VAL | A | 139 | −35.256 | −8.925 | 55.269 | 1.00 | 48.01 | O |
| ATOM | 194 | N | LEU | A | 140 | −36.198 | −10.913 | 55.032 | 1.00 | 40.11 | N |
| ATOM | 195 | CA | LEU | A | 140 | −34.947 | −11.643 | 55.085 | 1.00 | 44.81 | C |
| ATOM | 196 | CB | LEU | A | 140 | −35.182 | −13.141 | 54.886 | 1.00 | 41.93 | C |
| ATOM | 197 | CG | LEU | A | 140 | −35.813 | −13.873 | 56.068 | 1.00 | 44.13 | C |
| ATOM | 198 | CD1 | LEU | A | 140 | −35.987 | −15.341 | 55.743 | 1.00 | 51.06 | C |
| ATOM | 199 | CD2 | LEU | A | 140 | −34.976 | −13.694 | 57.325 | 1.00 | 43.56 | C |
| ATOM | 200 | C | LEU | A | 140 | −33.932 | −11.126 | 54.064 | 1.00 | 53.10 | C |
| ATOM | 201 | O | LEU | A | 140 | −32.800 | −10.821 | 54.410 | 1.00 | 55.71 | O |
| ATOM | 202 | N | PHE | A | 141 | −34.339 | −11.008 | 52.814 | 1.00 | 47.92 | N |
| ATOM | 203 | CA | PHE | A | 141 | −33.450 | −10.562 | 51.768 | 1.00 | 46.51 | C |
| ATOM | 204 | CB | PHE | A | 141 | −34.087 | −10.747 | 50.387 | 1.00 | 46.53 | C |
| ATOM | 205 | CG | PHE | A | 141 | −33.959 | −12.144 | 49.841 | 1.00 | 48.77 | C |
| ATOM | 206 | CD1 | PHE | A | 141 | −32.812 | −12.537 | 49.176 | 1.00 | 57.26 | C |
| ATOM | 207 | CE1 | PHE | A | 141 | −32.687 | −13.820 | 48.671 | 1.00 | 55.50 | C |
| ATOM | 208 | CZ | PHE | A | 141 | −33.715 | −14.724 | 48.825 | 1.00 | 56.84 | C |
| ATOM | 209 | CE2 | PHE | A | 141 | −34.865 | −14.349 | 49.485 | 1.00 | 51.70 | C |
| ATOM | 210 | CD2 | PHE | A | 141 | −34.985 | −13.062 | 49.988 | 1.00 | 47.91 | C |
| ATOM | 211 | C | PHE | A | 141 | −33.014 | −9.104 | 51.970 | 1.00 | 50.96 | C |
| ATOM | 212 | O | PHE | A | 141 | −31.868 | −8.775 | 51.780 | 1.00 | 57.75 | O |
| ATOM | 213 | N | LEU | A | 142 | −33.906 | −8.243 | 52.433 | 1.00 | 45.03 | N |
| ATOM | 214 | CA | LEU | A | 142 | −33.542 | −6.871 | 52.747 | 1.00 | 44.37 | C |
| ATOM | 215 | CB | LEU | A | 142 | −34.779 | −6.030 | 53.065 | 1.00 | 41.67 | C |
| ATOM | 216 | CG | LEU | A | 142 | −35.746 | −5.839 | 51.896 | 1.00 | 44.92 | C |
| ATOM | 217 | CD1 | LEU | A | 142 | −36.724 | −4.709 | 52.175 | 1.00 | 38.46 | C |
| ATOM | 218 | CD2 | LEU | A | 142 | −34.979 | −5.586 | 50.609 | 1.00 | 50.62 | C |
| ATOM | 219 | C | LEU | A | 142 | −32.515 | −6.773 | 53.879 | 1.00 | 54.88 | C |
| ATOM | 220 | O | LEU | A | 142 | −31.625 | −5.942 | 53.827 | 1.00 | 68.67 | O |
| ATOM | 221 | N | ARG | A | 143 | −32.676 | −7.577 | 54.939 | 1.00 | 51.67 | N |
| ATOM | 222 | CA | ARG | A | 143 | −31.752 | −7.534 | 56.082 | 1.00 | 53.13 | C |
| ATOM | 223 | CB | ARG | A | 143 | −32.283 | −8.364 | 57.258 | 1.00 | 57.58 | C |
| ATOM | 224 | CG | ARG | A | 143 | −33.590 | −7.863 | 57.865 | 1.00 | 65.60 | C |
| ATOM | 225 | CD | ARG | A | 143 | −33.829 | −8.423 | 59.288 | 1.00 | 71.13 | C |
| ATOM | 226 | NE | ARG | A | 143 | −34.372 | −7.367 | 60.146 | 1.00 | 66.17 | N |
| ATOM | 227 | CZ | ARG | A | 143 | −33.669 | −6.722 | 61.075 | 1.00 | 70.42 | C |
| ATOM | 228 | NH1 | ARG | A | 143 | −32.408 | −7.078 | 61.307 | 1.00 | 60.76 | N |
| ATOM | 229 | NH2 | ARG | A | 143 | −34.224 | −5.741 | 61.785 | 1.00 | 80.55 | N |
| ATOM | 230 | C | ARG | A | 143 | −30.346 | −7.965 | 55.713 | 1.00 | 57.33 | C |
| ATOM | 231 | O | ARG | A | 143 | −29.371 | −7.320 | 56.083 | 1.00 | 64.02 | O |
| ATOM | 232 | N | ALA | A | 144 | −30.266 | −9.045 | 54.939 | 1.00 | 62.65 | N |
| ATOM | 233 | CA | ALA | A | 144 | −29.011 | −9.561 | 54.416 | 1.00 | 71.25 | C |
| ATOM | 234 | CB | ALA | A | 144 | −29.283 | −10.630 | 53.357 | 1.00 | 62.32 | C |
| ATOM | 235 | C | ALA | A | 144 | −28.073 | −8.465 | 53.847 | 1.00 | 81.23 | C |
| ATOM | 236 | O | ALA | A | 144 | −28.494 | −7.594 | 53.081 | 1.00 | 83.83 | O |
| ATOM | 237 | O | ARG | A | 145 | −24.882 | −7.480 | 51.301 | 1.00 | 73.38 | O |
| ATOM | 238 | N | ARG | A | 145 | −26.775 | −8.594 | 54.147 | 1.00 | 78.24 | N |
| ATOM | 239 | CA | ARG | A | 145 | −25.765 | −7.735 | 53.543 | 1.00 | 77.31 | C |
| ATOM | 240 | C | ARG | A | 145 | −25.514 | −8.170 | 52.088 | 1.00 | 75.14 | C |
| ATOM | 241 | CB | ARG | A | 145 | −24.446 | −7.841 | 54.308 | 1.00 | 89.50 | C |
| ATOM | 242 | CG | ARG | A | 145 | −24.512 | −7.614 | 55.808 | 1.00 | 95.00 | C |
| ATOM | 243 | CD | ARG | A | 145 | −23.107 | −7.755 | 56.385 | 1.00 | 102.28 | C |
| ATOM | 244 | NE | ARG | A | 145 | −23.019 | −7.416 | 57.804 | 1.00 | 113.25 | N |
| ATOM | 245 | CZ | ARG | A | 145 | −21.874 | −7.345 | 58.481 | 1.00 | 109.58 | C |
| ATOM | 246 | NH1 | ARG | A | 145 | −20.725 | −7.588 | 57.865 | 1.00 | 93.40 | N |
| ATOM | 247 | NH2 | ARG | A | 145 | −21.871 | −7.026 | 59.769 | 1.00 | 114.11 | N |
| ATOM | 248 | O | SER | A | 146 | −26.714 | −9.589 | 48.237 | 1.00 | 64.99 | O |
| ATOM | 249 | N | SER | A | 146 | −26.012 | −9.354 | 51.747 | 1.00 | 76.07 | N |
| ATOM | 250 | CA | SER | A | 146 | −25.833 | −9.901 | 50.417 | 1.00 | 72.08 | C |
| ATOM | 251 | C | SER | A | 146 | −26.837 | −9.329 | 49.416 | 1.00 | 67.28 | C |
| ATOM | 252 | CB | SER | A | 146 | −25.923 | −11.433 | 50.436 | 1.00 | 68.91 | C |
| ATOM | 253 | OG | SER | A | 146 | −27.230 | −11.873 | 50.755 | 1.00 | 75.03 | O |
| ATOM | 254 | N | ILE | A | 147 | −27.752 | −8.450 | 49.878 | 1.00 | 67.52 | N |
| ATOM | 255 | CA | ILE | A | 147 | −28.727 | −7.792 | 48.994 | 1.00 | 66.81 | C |
| ATOM | 256 | CB | ILE | A | 147 | −29.654 | −6.842 | 49.782 | 1.00 | 62.35 | C |
| ATOM | 257 | CG1 | ILE | A | 147 | −30.830 | −6.402 | 48.911 | 1.00 | 58.57 | C |
| ATOM | 258 | CD1 | ILE | A | 147 | −31.630 | −7.560 | 48.367 | 1.00 | 55.25 | C |
| ATOM | 259 | CG2 | ILE | A | 147 | −28.876 | −5.649 | 50.326 | 1.00 | 61.58 | C |
| ATOM | 260 | C | ILE | A | 147 | −28.041 | −7.031 | 47.824 | 1.00 | 64.06 | C |
| ATOM | 261 | O | ILE | A | 147 | −28.549 | −6.948 | 46.713 | 1.00 | 61.14 | O |
| ATOM | 262 | N | ARG | A | 148 | −26.782 | −6.677 | 48.075 | 1.00 | 66.52 | N |
| ATOM | 263 | CA | ARG | A | 148 | −25.809 | −6.182 | 47.100 | 1.00 | 62.39 | C |
| ATOM | 264 | C | ARG | A | 148 | −25.499 | −7.136 | 45.896 | 1.00 | 52.25 | C |
| ATOM | 265 | O | ARG | A | 148 | −25.435 | −6.694 | 44.762 | 1.00 | 47.43 | O |
| ATOM | 266 | CB | ARG | A | 148 | −24.551 | −5.695 | 47.821 | 1.00 | 70.47 | C |
| ATOM | 267 | CG | ARG | A | 148 | −24.803 | −4.427 | 48.642 | 1.00 | 74.52 | C |
| ATOM | 268 | CD | ARG | A | 148 | −23.895 | −4.314 | 49.864 | 1.00 | 79.58 | C |
| ATOM | 269 | NE | ARG | A | 148 | −24.276 | −3.180 | 50.705 | 1.00 | 81.57 | N |
| ATOM | 270 | CZ | ARG | A | 148 | −23.661 | −2.836 | 51.834 | 1.00 | 89.85 | C |
| ATOM | 271 | NH1 | ARG | A | 148 | −22.623 | −3.537 | 52.270 | 1.00 | 81.85 | N |
| ATOM | 272 | NH2 | ARG | A | 148 | −24.085 | −1.786 | 52.527 | 1.00 | 99.17 | N |
| ATOM | 273 | N | CYS | A | 149 | −25.373 | −8.454 | 46.161 | 1.00 | 48.83 | N |
| ATOM | 274 | CA | CYS | A | 149 | −25.186 | −9.470 | 45.109 | 1.00 | 52.63 | C |
| ATOM | 275 | CB | CYS | A | 149 | −25.155 | −10.882 | 45.706 | 1.00 | 53.34 | C |
| ATOM | 276 | SG | CYS | A | 149 | −23.778 | −11.262 | 46.803 | 1.00 | 54.84 | S |
| ATOM | 277 | C | CYS | A | 149 | −26.309 | −9.425 | 44.064 | 1.00 | 57.52 | C |
| ATOM | 278 | O | CYS | A | 149 | −27.440 | −9.183 | 44.395 | 1.00 | 59.34 | O |
| ATOM | 279 | N | LEU | A | 150 | −26.006 | −9.721 | 42.808 | 1.00 | 55.50 | N |
| ATOM | 280 | CA | LEU | A | 150 | −27.031 | −9.776 | 41.783 | 1.00 | 52.64 | C |
| ATOM | 281 | CB | LEU | A | 150 | −26.405 | −9.902 | 40.391 | 1.00 | 43.47 | C |
| ATOM | 282 | CG | LEU | A | 150 | −27.337 | −9.606 | 39.215 | 1.00 | 44.97 | C |
| ATOM | 283 | CD1 | LEU | A | 150 | −27.868 | −8.183 | 39.290 | 1.00 | 45.69 | C |
| ATOM | 284 | CD2 | LEU | A | 150 | −26.636 | −9.844 | 37.892 | 1.00 | 43.90 | C |
| ATOM | 285 | C | LEU | A | 150 | −28.065 | −10.875 | 42.013 | 1.00 | 58.28 | C |
| ATOM | 286 | O | LEU | A | 150 | −29.230 | −10.726 | 41.687 | 1.00 | 51.99 | O |
| ATOM | 287 | N | ARG | A | 151 | −27.650 | −11.973 | 42.641 | 1.00 | 62.56 | N |
| ATOM | 288 | CA | ARG | A | 151 | −28.566 | −13.069 | 42.931 | 1.00 | 54.40 | C |
| ATOM | 289 | CB | ARG | A | 151 | −27.875 | −14.170 | 43.736 | 1.00 | 59.04 | C |
| ATOM | 290 | CG | ARG | A | 151 | −26.839 | −14.967 | 42.992 | 1.00 | 67.34 | C |
| ATOM | 291 | CD | ARG | A | 151 | −26.335 | −16.148 | 43.829 | 1.00 | 72.28 | C |
| ATOM | 292 | NE | ARG | A | 151 | −25.835 | −15.720 | 45.137 | 1.00 | 72.03 | N |
| ATOM | 293 | CZ | ARG | A | 151 | −26.402 | −16.038 | 46.299 | 1.00 | 79.71 | C |
| ATOM | 294 | NH1 | ARG | A | 151 | −25.890 | −15.592 | 47.441 | 1.00 | 78.41 | N |
| ATOM | 295 | NH2 | ARG | A | 151 | −27.483 | −16.807 | 46.323 | 1.00 | 84.71 | N |
| ATOM | 296 | C | ARG | A | 151 | −29.726 | −12.586 | 43.763 | 1.00 | 54.76 | C |
| ATOM | 297 | O | ARG | A | 151 | −30.862 | −12.836 | 43.442 | 1.00 | 57.87 | O |
| ATOM | 298 | N | ASN | A | 152 | −29.392 | −11.933 | 44.868 | 1.00 | 53.78 | N |
| ATOM | 299 | CA | ASN | A | 152 | −30.355 | −11.464 | 45.822 | 1.00 | 54.88 | C |
| ATOM | 300 | CB | ASN | A | 152 | −29.662 | −11.043 | 47.119 | 1.00 | 61.05 | C |
| ATOM | 301 | CG | ASN | A | 152 | −29.023 | −12.214 | 47.845 | 1.00 | 68.32 | C |
| ATOM | 302 | OD1 | ASN | A | 152 | −27.972 | −12.713 | 47.445 | 1.00 | 69.78 | O |
| ATOM | 303 | ND2 | ASN | A | 152 | −29.653 | −12.650 | 48.929 | 1.00 | 74.75 | N |
| ATOM | 304 | C | ASN | A | 152 | −31.225 | −10.323 | 45.284 | 1.00 | 54.27 | C |
| ATOM | 305 | O | ASN | A | 152 | −32.364 | −10.194 | 45.669 | 1.00 | 49.93 | O |
| ATOM | 306 | N | ILE | A | 153 | −30.705 | −9.491 | 44.378 | 1.00 | 53.40 | N |
| ATOM | 307 | CA | ILE | A | 153 | −31.517 | −8.402 | 43.828 | 1.00 | 49.58 | C |
| ATOM | 308 | CB | ILE | A | 153 | −30.697 | −7.480 | 42.915 | 1.00 | 43.94 | C |
| ATOM | 309 | CG1 | ILE | A | 153 | −29.551 | −6.840 | 43.691 | 1.00 | 49.94 | C |
| ATOM | 310 | CD1 | ILE | A | 153 | −28.756 | −5.844 | 42.878 | 1.00 | 55.29 | C |
| ATOM | 311 | CG2 | ILE | A | 153 | −31.583 | −6.401 | 42.322 | 1.00 | 46.47 | C |
| ATOM | 312 | C | ILE | A | 153 | −32.678 | −8.957 | 43.028 | 1.00 | 48.34 | C |
| ATOM | 313 | O | ILE | A | 153 | −33.812 | −8.558 | 43.199 | 1.00 | 46.75 | O |
| ATOM | 314 | N | ILE | A | 154 | −32.377 | −9.945 | 42.199 | 1.00 | 49.04 | N |
| ATOM | 315 | CA | ILE | A | 154 | −33.388 | −10.658 | 41.455 | 1.00 | 43.90 | C |
| ATOM | 316 | CB | ILE | A | 154 | −32.774 | −11.619 | 40.427 | 1.00 | 36.97 | C |
| ATOM | 317 | CG1 | ILE | A | 154 | −31.918 | −10.829 | 39.441 | 1.00 | 37.63 | C |
| ATOM | 318 | CD1 | ILE | A | 154 | −31.402 | −11.655 | 38.293 | 1.00 | 43.82 | C |
| ATOM | 319 | CG2 | ILE | A | 154 | −33.860 | −12.364 | 39.672 | 1.00 | 41.40 | C |
| ATOM | 320 | C | ILE | A | 154 | −34.391 | −11.387 | 42.356 | 1.00 | 46.22 | C |
| ATOM | 321 | O | ILE | A | 154 | −35.571 | −11.354 | 42.099 | 1.00 | 47.42 | O |
| ATOM | 322 | N | HIS | A | 155 | −33.921 | −12.066 | 43.404 | 1.00 | 46.77 | N |
| ATOM | 323 | CA | HIS | A | 155 | −34.800 | −12.816 | 44.304 | 1.00 | 44.83 | C |
| ATOM | 324 | CB | HIS | A | 155 | −33.973 | −13.604 | 45.318 | 1.00 | 48.36 | C |
| ATOM | 325 | CG | HIS | A | 155 | −33.273 | −14.791 | 44.736 | 1.00 | 52.09 | C |
| ATOM | 326 | ND1 | HIS | A | 155 | −31.933 | −15.034 | 44.934 | 1.00 | 54.23 | N |
| ATOM | 327 | CE1 | HIS | A | 155 | −31.596 | −16.149 | 44.307 | 1.00 | 52.50 | C |
| ATOM | 328 | NE2 | HIS | A | 155 | −32.670 | −16.634 | 43.714 | 1.00 | 49.83 | N |
| ATOM | 329 | CD2 | HIS | A | 155 | −33.737 | −15.807 | 43.975 | 1.00 | 54.18 | C |
| ATOM | 330 | C | HIS | A | 155 | −35.725 | −11.918 | 45.059 | 1.00 | 45.06 | C |
| ATOM | 331 | O | HIS | A | 155 | −36.883 | −12.187 | 45.171 | 1.00 | 45.84 | O |
| ATOM | 332 | N | ALA | A | 156 | −35.196 | −10.835 | 45.575 | 1.00 | 46.31 | N |
| ATOM | 333 | CA | ALA | A | 156 | −35.988 | −9.851 | 46.283 | 1.00 | 48.40 | C |
| ATOM | 334 | CB | ALA | A | 156 | −35.086 | −8.780 | 46.888 | 1.00 | 48.09 | C |
| ATOM | 335 | C | ALA | A | 156 | −37.057 | −9.212 | 45.402 | 1.00 | 45.74 | C |
| ATOM | 336 | O | ALA | A | 156 | −38.196 | −9.125 | 45.763 | 1.00 | 41.56 | O |
| ATOM | 337 | N | ASN | A | 157 | −36.695 | −8.842 | 44.196 | 1.00 | 45.83 | N |
| ATOM | 338 | CA | ASN | A | 157 | −37.659 | −8.318 | 43.240 | 1.00 | 45.43 | C |
| ATOM | 339 | CB | ASN | A | 157 | −36.938 | −7.853 | 41.979 | 1.00 | 48.02 | C |
| ATOM | 340 | CG | ASN | A | 157 | −36.363 | −6.460 | 42.106 | 1.00 | 50.49 | C |
| ATOM | 341 | OD1 | ASN | A | 157 | −37.062 | −5.466 | 41.900 | 1.00 | 46.72 | O |
| ATOM | 342 | ND2 | ASN | A | 157 | −35.076 | −6.380 | 42.426 | 1.00 | 54.11 | N |
| ATOM | 343 | C | ASN | A | 157 | −38.682 | −9.374 | 42.844 | 1.00 | 42.91 | C |
| ATOM | 344 | O | ASN | A | 157 | −39.849 | −9.101 | 42.744 | 1.00 | 46.16 | O |
| ATOM | 345 | N | LEU | A | 158 | −38.238 | −10.603 | 42.690 | 1.00 | 41.87 | N |
| ATOM | 346 | CA | LEU | A | 158 | −39.116 | −11.731 | 42.413 | 1.00 | 42.61 | C |
| ATOM | 347 | CB | LEU | A | 158 | −38.281 | −12.997 | 42.206 | 1.00 | 44.46 | C |
| ATOM | 348 | CG | LEU | A | 158 | −38.973 | −14.359 | 42.150 | 1.00 | 40.44 | C |
| ATOM | 349 | CD1 | LEU | A | 158 | −39.834 | −14.482 | 40.908 | 1.00 | 38.70 | C |
| ATOM | 350 | CD2 | LEU | A | 158 | −37.930 | −15.463 | 42.189 | 1.00 | 49.66 | C |
| ATOM | 351 | C | LEU | A | 158 | −40.123 | −11.957 | 43.544 | 1.00 | 44.36 | C |
| ATOM | 352 | O | LEU | A | 158 | −41.306 | −12.090 | 43.315 | 1.00 | 41.88 | O |
| ATOM | 353 | N | ILE | A | 159 | −39.663 | −11.899 | 44.778 | 1.00 | 40.19 | N |
| ATOM | 354 | CA | ILE | A | 159 | −40.546 | −11.980 | 45.916 | 1.00 | 40.08 | C |
| ATOM | 355 | CB | ILE | A | 159 | −39.758 | −12.074 | 47.227 | 1.00 | 41.27 | C |
| ATOM | 356 | CG1 | ILE | A | 159 | −39.060 | −13.432 | 47.294 | 1.00 | 44.90 | C |
| ATOM | 357 | CD1 | ILE | A | 159 | −38.093 | −13.563 | 48.438 | 1.00 | 46.14 | C |
| ATOM | 358 | CG2 | ILE | A | 159 | −40.677 | −11.892 | 48.428 | 1.00 | 35.69 | C |
| ATOM | 359 | C | ILE | A | 159 | −41.548 | −10.839 | 45.958 | 1.00 | 44.60 | C |
| ATOM | 360 | O | ILE | A | 159 | −42.715 | −11.065 | 46.139 | 1.00 | 40.05 | O |
| ATOM | 361 | N | ALA | A | 160 | −41.097 | −9.611 | 45.731 | 1.00 | 49.39 | N |
| ATOM | 362 | CA | ALA | A | 160 | −41.971 | −8.449 | 45.694 | 1.00 | 39.03 | C |
| ATOM | 363 | CB | ALA | A | 160 | −41.169 | −7.177 | 45.467 | 1.00 | 40.10 | C |
| ATOM | 364 | C | ALA | A | 160 | −43.035 | −8.599 | 44.654 | 1.00 | 39.28 | C |
| ATOM | 365 | O | ALA | A | 160 | −44.174 | −8.304 | 44.882 | 1.00 | 44.22 | O |
| ATOM | 366 | N | ALA | A | 161 | −42.661 | −9.049 | 43.488 | 1.00 | 36.12 | N |
| ATOM | 367 | CA | ALA | A | 161 | −43.619 | −9.237 | 42.424 | 1.00 | 41.74 | C |
| ATOM | 368 | CB | ALA | A | 161 | −42.907 | −9.716 | 41.174 | 1.00 | 36.79 | C |
| ATOM | 369 | C | ALA | A | 161 | −44.746 | −10.213 | 42.794 | 1.00 | 46.51 | C |
| ATOM | 370 | O | ALA | A | 161 | −45.913 | −9.929 | 42.620 | 1.00 | 47.56 | O |
| ATOM | 371 | N | PHE | A | 162 | −44.355 | −11.343 | 43.371 | 1.00 | 43.25 | N |
| ATOM | 372 | CA | PHE | A | 162 | −45.237 | −12.275 | 44.032 | 1.00 | 36.17 | C |
| ATOM | 373 | CB | PHE | A | 162 | −44.513 | −13.570 | 44.389 | 1.00 | 32.84 | C |
| ATOM | 374 | CG | PHE | A | 162 | −44.408 | −14.529 | 43.235 | 1.00 | 36.48 | C |
| ATOM | 375 | CD1 | PHE | A | 162 | −45.356 | −15.517 | 43.056 | 1.00 | 42.88 | C |
| ATOM | 376 | CE1 | PHE | A | 162 | −45.272 | −16.402 | 41.996 | 1.00 | 42.57 | C |
| ATOM | 377 | CZ | PHE | A | 162 | −44.239 | −16.305 | 41.098 | 1.00 | 37.38 | C |
| ATOM | 378 | CE2 | PHE | A | 162 | −43.290 | −15.322 | 41.257 | 1.00 | 40.58 | C |
| ATOM | 379 | CD2 | PHE | A | 162 | −43.376 | −14.434 | 42.319 | 1.00 | 39.78 | C |
| ATOM | 380 | C | PHE | A | 162 | −46.038 | −11.700 | 45.201 | 1.00 | 40.22 | C |
| ATOM | 381 | O | PHE | A | 162 | −47.179 | −12.021 | 45.338 | 1.00 | 41.48 | O |
| ATOM | 382 | N | ILE | A | 163 | −45.462 | −10.843 | 46.052 | 1.00 | 41.07 | N |
| ATOM | 383 | CA | ILE | A | 163 | −46.222 | −10.269 | 47.166 | 1.00 | 35.93 | C |
| ATOM | 384 | CB | ILE | A | 163 | −45.345 | −9.347 | 48.042 | 1.00 | 33.43 | C |
| ATOM | 385 | CG1 | ILE | A | 163 | −44.326 | −10.165 | 48.824 | 1.00 | 36.85 | C |
| ATOM | 386 | CD1 | ILE | A | 163 | −43.464 | −9.328 | 49.735 | 1.00 | 44.10 | C |
| ATOM | 387 | CG2 | ILE | A | 163 | −46.197 | −8.535 | 49.012 | 1.00 | 30.87 | C |
| ATOM | 388 | C | ILE | A | 163 | −47.366 | −9.467 | 46.650 | 1.00 | 36.92 | C |
| ATOM | 389 | O | ILE | A | 163 | −48.466 | −9.587 | 47.114 | 1.00 | 42.62 | O |
| ATOM | 390 | N | LEU | A | 164 | −47.087 | −8.628 | 45.679 | 1.00 | 37.26 | N |
| ATOM | 391 | CA | LEU | A | 164 | −48.081 | −7.764 | 45.115 | 1.00 | 39.98 | C |
| ATOM | 392 | CB | LEU | A | 164 | −47.472 | −6.837 | 44.058 | 1.00 | 46.09 | C |
| ATOM | 393 | CG | LEU | A | 164 | −46.578 | −5.731 | 44.633 | 1.00 | 47.69 | C |
| ATOM | 394 | CD1 | LEU | A | 164 | −45.885 | −4.947 | 43.530 | 1.00 | 48.29 | C |
| ATOM | 395 | CD2 | LEU | A | 164 | −47.371 | −4.796 | 45.542 | 1.00 | 32.81 | C |
| ATOM | 396 | C | LEU | A | 164 | −49.239 | −8.519 | 44.559 | 1.00 | 44.13 | C |
| ATOM | 397 | O | LEU | A | 164 | −50.362 | −8.161 | 44.783 | 1.00 | 49.75 | O |
| ATOM | 398 | N | ARG | A | 165 | −48.988 | −9.591 | 43.857 | 1.00 | 41.11 | N |
| ATOM | 399 | CA | ARG | A | 165 | −50.089 | −10.359 | 43.345 | 1.00 | 45.43 | C |
| ATOM | 400 | CB | ARG | A | 165 | −49.567 | −11.474 | 42.428 | 1.00 | 42.05 | C |
| ATOM | 401 | CG | ARG | A | 165 | −50.635 | −12.467 | 42.011 | 1.00 | 43.15 | C |
| ATOM | 402 | CD | ARG | A | 165 | −50.122 | −13.419 | 40.964 | 1.00 | 48.40 | C |
| ATOM | 403 | NE | ARG | A | 165 | −51.162 | −14.311 | 40.467 | 1.00 | 48.65 | N |
| ATOM | 404 | CZ | ARG | A | 165 | −52.090 | −13.947 | 39.590 | 1.00 | 49.86 | C |
| ATOM | 405 | NH1 | ARG | A | 165 | −52.122 | −12.700 | 39.134 | 1.00 | 48.22 | N |
| ATOM | 406 | NH2 | ARG | A | 165 | −52.991 | −14.827 | 39.178 | 1.00 | 53.90 | N |
| ATOM | 407 | C | ARG | A | 165 | −51.012 | −10.969 | 44.410 | 1.00 | 45.12 | C |
| ATOM | 408 | O | ARG | A | 165 | −52.207 | −10.852 | 44.313 | 1.00 | 49.67 | O |
| ATOM | 409 | N | ASN | A | 166 | −50.454 | −11.561 | 45.454 | 1.00 | 43.44 | N |
| ATOM | 410 | C | ASN | A | 166 | −52.083 | −11.079 | 47.296 | 1.00 | 45.48 | C |
| ATOM | 411 | O | ASN | A | 166 | −53.207 | −11.343 | 47.646 | 1.00 | 53.19 | O |
| ATOM | 412 | CA | ASN | A | 166 | −51.220 | −12.101 | 46.580 | 1.00 | 39.93 | C |
| ATOM | 413 | CB | ASN | A | 166 | −50.317 | −12.856 | 47.547 | 1.00 | 43.27 | C |
| ATOM | 414 | CG | ASN | A | 166 | −49.965 | −14.238 | 47.037 | 1.00 | 51.94 | C |
| ATOM | 415 | OD1 | ASN | A | 166 | −49.203 | −14.387 | 46.076 | 1.00 | 48.74 | O |
| ATOM | 416 | ND2 | ASN | A | 166 | −50.523 | −15.260 | 47.676 | 1.00 | 52.73 | N |
| ATOM | 417 | N | ALA | A | 167 | −51.541 | −9.903 | 47.528 | 1.00 | 37.55 | N |
| ATOM | 418 | CA | ALA | A | 167 | −52.286 | −8.854 | 48.161 | 1.00 | 37.09 | C |
| ATOM | 419 | CB | ALA | A | 167 | −51.367 | −7.685 | 48.481 | 1.00 | 38.73 | C |
| ATOM | 420 | C | ALA | A | 167 | −53.450 | −8.385 | 47.292 | 1.00 | 44.10 | C |
| ATOM | 421 | O | ALA | A | 167 | −54.550 | −8.180 | 47.751 | 1.00 | 45.93 | O |
| ATOM | 422 | N | THR | A | 168 | −53.192 | −8.245 | 45.997 | 1.00 | 47.39 | N |
| ATOM | 423 | CA | THR | A | 168 | −54.212 | −7.869 | 45.028 | 1.00 | 45.52 | C |
| ATOM | 424 | CB | THR | A | 168 | −53.599 | −7.602 | 43.624 | 1.00 | 42.11 | C |
| ATOM | 425 | OG1 | THR | A | 168 | −52.523 | −6.664 | 43.732 | 1.00 | 39.71 | O |
| ATOM | 426 | CG2 | THR | A | 168 | −54.641 | −7.030 | 42.680 | 1.00 | 46.17 | C |
| ATOM | 427 | C | THR | A | 168 | −55.302 | −8.894 | 44.911 | 1.00 | 44.30 | C |
| ATOM | 428 | O | THR | A | 168 | −56.427 | −8.532 | 44.695 | 1.00 | 44.28 | O |
| ATOM | 429 | N | TRP | A | 169 | −54.964 | −10.165 | 45.107 | 1.00 | 39.21 | N |
| ATOM | 430 | CA | TRP | A | 169 | −55.941 | −11.237 | 45.160 | 1.00 | 41.96 | C |
| ATOM | 431 | CB | TRP | A | 169 | −55.269 | −12.565 | 45.502 | 1.00 | 41.28 | C |
| ATOM | 432 | CG | TRP | A | 169 | −56.183 | −13.744 | 45.434 | 1.00 | 43.27 | C |
| ATOM | 433 | CD1 | TRP | A | 169 | −56.383 | −14.553 | 44.360 | 1.00 | 43.66 | C |
| ATOM | 434 | NE1 | TRP | A | 169 | −57.285 | −15.536 | 44.668 | 1.00 | 47.46 | N |
| ATOM | 435 | CE2 | TRP | A | 169 | −57.687 | −15.379 | 45.967 | 1.00 | 42.99 | C |
| ATOM | 436 | CD2 | TRP | A | 169 | −57.012 | −14.257 | 46.485 | 1.00 | 42.42 | C |
| ATOM | 437 | CE3 | TRP | A | 169 | −57.250 | −13.880 | 47.809 | 1.00 | 44.33 | C |
| ATOM | 438 | CZ3 | TRP | A | 169 | −58.141 | −14.627 | 48.557 | 1.00 | 48.17 | C |
| ATOM | 439 | CH2 | TRP | A | 169 | −58.796 | −15.737 | 48.009 | 1.00 | 45.44 | C |
| ATOM | 440 | CZ2 | TRP | A | 169 | −58.581 | −16.126 | 46.720 | 1.00 | 43.64 | C |
| ATOM | 441 | C | TRP | A | 169 | −57.056 | −10.922 | 46.147 | 1.00 | 46.33 | C |
| ATOM | 442 | O | TRP | A | 169 | −58.211 | −11.064 | 45.842 | 1.00 | 46.29 | O |
| ATOM | 443 | N | PHE | A | 170 | −56.707 | −10.509 | 47.349 | 1.00 | 42.17 | N |
| ATOM | 444 | CA | PHE | A | 170 | −57.708 | −10.169 | 48.325 | 1.00 | 42.75 | C |
| ATOM | 445 | CB | PHE | A | 170 | −57.054 | −9.877 | 49.678 | 1.00 | 37.58 | C |
| ATOM | 446 | CG | PHE | A | 170 | −56.355 | −11.066 | 50.275 | 1.00 | 37.59 | C |
| ATOM | 447 | CD1 | PHE | A | 170 | −57.070 | −12.043 | 50.945 | 1.00 | 42.53 | C |
| ATOM | 448 | CE1 | PHE | A | 170 | −56.433 | −13.140 | 51.491 | 1.00 | 40.04 | C |
| ATOM | 449 | CZ | PHE | A | 170 | −55.069 | −13.272 | 51.371 | 1.00 | 46.68 | C |
| ATOM | 450 | CE2 | PHE | A | 170 | −54.343 | −12.308 | 50.705 | 1.00 | 47.27 | C |
| ATOM | 451 | CD2 | PHE | A | 170 | −54.987 | −11.212 | 50.161 | 1.00 | 46.81 | C |
| ATOM | 452 | C | PHE | A | 170 | −58.637 | −9.012 | 47.881 | 1.00 | 46.88 | C |
| ATOM | 453 | O | PHE | A | 170 | −59.846 | −9.071 | 48.031 | 1.00 | 50.33 | O |
| ATOM | 454 | N | VAL | A | 171 | −58.072 | −8.006 | 47.224 | 1.00 | 43.21 | N |
| ATOM | 455 | CA | VAL | A | 171 | −58.868 | −6.954 | 46.571 | 1.00 | 44.02 | C |
| ATOM | 456 | CB | VAL | A | 171 | −57.976 | −5.814 | 46.023 | 1.00 | 40.17 | C |
| ATOM | 457 | CG1 | VAL | A | 171 | −58.824 | −4.673 | 45.510 | 1.00 | 31.79 | C |
| ATOM | 458 | CG2 | VAL | A | 171 | −57.054 | −5.305 | 47.115 | 1.00 | 37.81 | C |
| ATOM | 459 | C | VAL | A | 171 | −59.835 | −7.502 | 45.477 | 1.00 | 43.54 | C |
| ATOM | 460 | O | VAL | A | 171 | −61.006 | −7.192 | 45.407 | 1.00 | 49.34 | O |
| ATOM | 461 | N | VAL | A | 172 | −59.312 | −8.360 | 44.629 | 1.00 | 45.50 | N |
| ATOM | 462 | CA | VAL | A | 172 | −60.035 | −9.018 | 43.551 | 1.00 | 48.68 | C |
| ATOM | 463 | CB | VAL | A | 172 | −59.112 | −9.996 | 42.784 | 1.00 | 44.76 | C |
| ATOM | 464 | CG1 | VAL | A | 172 | −59.916 | −10.960 | 41.914 | 1.00 | 44.92 | C |
| ATOM | 465 | CG2 | VAL | A | 172 | −58.112 | −9.222 | 41.951 | 1.00 | 45.02 | C |
| ATOM | 466 | C | VAL | A | 172 | −61.245 | −9.775 | 44.071 | 1.00 | 51.42 | C |
| ATOM | 467 | O | VAL | A | 172 | −62.312 | −9.746 | 43.497 | 1.00 | 57.46 | O |
| ATOM | 468 | N | GLN | A | 173 | −61.084 | −10.383 | 45.226 | 1.00 | 52.22 | N |
| ATOM | 469 | CA | GLN | A | 173 | −62.174 | −11.019 | 45.940 | 1.00 | 53.55 | C |
| ATOM | 470 | CB | GLN | A | 173 | −61.699 | −11.594 | 47.274 | 1.00 | 51.62 | C |
| ATOM | 471 | CG | GLN | A | 173 | −60.797 | −12.797 | 47.111 | 1.00 | 48.65 | C |
| ATOM | 472 | CD | GLN | A | 173 | −61.471 | −13.914 | 46.340 | 1.00 | 50.78 | C |
| ATOM | 473 | OE1 | GLN | A | 173 | −61.078 | −14.239 | 45.216 | 1.00 | 52.72 | O |
| ATOM | 474 | NE2 | GLN | A | 173 | −62.496 | −14.508 | 46.939 | 1.00 | 46.74 | N |
| ATOM | 475 | C | GLN | A | 173 | −63.361 | −10.066 | 46.142 | 1.00 | 53.82 | C |
| ATOM | 476 | O | GLN | A | 173 | −64.504 | −10.443 | 45.918 | 1.00 | 51.66 | O |
| ATOM | 477 | N | LEU | A | 174 | −63.078 | −8.802 | 46.491 | 1.00 | 53.50 | N |
| ATOM | 478 | CA | LEU | A | 174 | −64.165 | −7.804 | 46.609 | 1.00 | 54.55 | C |
| ATOM | 479 | CB | LEU | A | 174 | −63.626 | −6.468 | 47.131 | 1.00 | 43.01 | C |
| ATOM | 480 | CG | LEU | A | 174 | −62.718 | −6.460 | 48.361 | 1.00 | 40.80 | C |
| ATOM | 481 | CD1 | LEU | A | 174 | −62.425 | −5.028 | 48.785 | 1.00 | 26.31 | C |
| ATOM | 482 | CD2 | LEU | A | 174 | −63.326 | −7.253 | 49.509 | 1.00 | 44.43 | C |
| ATOM | 483 | C | LEU | A | 174 | −64.888 | −7.558 | 45.257 | 1.00 | 56.64 | C |
| ATOM | 484 | O | LEU | A | 174 | −66.096 | −7.379 | 45.228 | 1.00 | 62.29 | O |
| ATOM | 485 | N | THR | A | 175 | −64.143 | −7.567 | 44.142 | 1.00 | 54.51 | N |
| ATOM | 486 | CA | THR | A | 175 | −64.754 | −7.365 | 42.820 | 1.00 | 57.39 | C |
| ATOM | 487 | CB | THR | A | 175 | −63.690 | −7.256 | 41.700 | 1.00 | 55.56 | C |
| ATOM | 488 | OG1 | THR | A | 175 | −63.009 | −8.508 | 41.549 | 1.00 | 57.65 | O |
| ATOM | 489 | CG2 | THR | A | 175 | −62.681 | −6.166 | 42.022 | 1.00 | 51.77 | C |
| ATOM | 490 | C | THR | A | 175 | −65.806 | −8.404 | 42.411 | 1.00 | 56.90 | C |
| ATOM | 491 | O | THR | A | 175 | −66.671 | −8.099 | 41.621 | 1.00 | 58.65 | O |
| ATOM | 492 | N | MET | A | 176 | −65.617 | −9.602 | 42.963 | 1.00 | 56.28 | N |
| ATOM | 493 | CA | MET | A | 176 | −66.321 | −10.834 | 42.638 | 1.00 | 57.99 | C |
| ATOM | 494 | CB | MET | A | 176 | −65.698 | −12.025 | 43.371 | 1.00 | 62.70 | C |
| ATOM | 495 | CG | MET | A | 176 | −64.308 | −12.389 | 42.884 | 1.00 | 59.78 | C |
| ATOM | 496 | SD | MET | A | 176 | −64.301 | −12.906 | 41.158 | 1.00 | 82.43 | S |
| ATOM | 497 | CE | MET | A | 176 | −65.213 | −14.444 | 41.253 | 1.00 | 65.78 | C |
| ATOM | 498 | C | MET | A | 176 | −67.802 | −10.759 | 42.936 | 1.00 | 61.28 | C |
| ATOM | 499 | O | MET | A | 176 | −68.617 | −11.335 | 42.215 | 1.00 | 67.67 | O |
| ATOM | 500 | O | SER | A | 177 | −69.923 | −8.626 | 42.521 | 1.00 | 64.27 | O |
| ATOM | 501 | N | SER | A | 177 | −68.158 | −10.055 | 44.007 | 1.00 | 66.17 | N |
| ATOM | 502 | CA | SER | A | 177 | −69.545 | −10.013 | 44.438 | 1.00 | 69.58 | C |
| ATOM | 503 | C | SER | A | 177 | −70.353 | −9.524 | 43.252 | 1.00 | 68.40 | C |
| ATOM | 504 | CB | SER | A | 177 | −69.723 | −9.034 | 45.597 | 1.00 | 68.15 | C |
| ATOM | 505 | OG | SER | A | 177 | −69.906 | −7.709 | 45.118 | 1.00 | 52.63 | O |
| ATOM | 506 | O | PRO | A | 178 | −72.225 | −8.068 | 40.277 | 1.00 | 70.57 | O |
| ATOM | 507 | N | PRO | A | 178 | −71.571 | −10.188 | 43.052 | 1.00 | 76.24 | N |
| ATOM | 508 | CA | PRO | A | 178 | −72.127 | −9.959 | 41.710 | 1.00 | 73.41 | C |
| ATOM | 509 | C | PRO | A | 178 | −72.425 | −8.502 | 41.411 | 1.00 | 73.28 | C |
| ATOM | 510 | CB | PRO | A | 178 | −73.429 | −10.770 | 41.734 | 1.00 | 74.07 | C |
| ATOM | 511 | CG | PRO | A | 178 | −73.784 | −10.877 | 43.189 | 1.00 | 78.90 | C |
| ATOM | 512 | CD | PRO | A | 178 | −72.483 | −10.948 | 43.930 | 1.00 | 77.40 | C |
| ATOM | 513 | O | GLU | A | 179 | −72.711 | −4.700 | 40.590 | 1.00 | 63.21 | O |
| ATOM | 514 | N | GLU | A | 179 | −72.919 | −7.761 | 42.393 | 1.00 | 70.86 | N |
| ATOM | 515 | CA | GLU | A | 179 | −73.489 | −6.456 | 42.090 | 1.00 | 65.98 | C |
| ATOM | 516 | C | GLU | A | 179 | −72.437 | −5.449 | 41.522 | 1.00 | 68.02 | C |
| ATOM | 517 | CB | GLU | A | 179 | −74.114 | −5.875 | 43.356 | 1.00 | 68.28 | C |
| ATOM | 518 | CG | GLU | A | 179 | −74.800 | −6.920 | 44.221 | 1.00 | 73.90 | C |
| ATOM | 519 | CD | GLU | A | 179 | −74.943 | −6.479 | 45.665 | 1.00 | 85.72 | C |
| ATOM | 520 | OE1 | GLU | A | 179 | −74.565 | −7.258 | 46.566 | 1.00 | 84.88 | O |
| ATOM | 521 | OE2 | GLU | A | 179 | −75.433 | −5.355 | 45.900 | 1.00 | 87.67 | O |
| ATOM | 522 | N | VAL | A | 180 | −71.194 | −5.540 | 42.057 | 1.00 | 69.54 | N |
| ATOM | 523 | CA | VAL | A | 180 | −69.982 | −4.837 | 41.572 | 1.00 | 64.98 | C |
| ATOM | 524 | CB | VAL | A | 180 | −68.858 | −4.898 | 42.629 | 1.00 | 62.11 | C |
| ATOM | 525 | CG1 | VAL | A | 180 | −67.662 | −4.069 | 42.189 | 1.00 | 66.05 | C |
| ATOM | 526 | CG2 | VAL | A | 180 | −69.374 | −4.417 | 43.978 | 1.00 | 60.37 | C |
| ATOM | 527 | C | VAL | A | 180 | −69.441 | −5.331 | 40.243 | 1.00 | 58.51 | C |
| ATOM | 528 | O | VAL | A | 180 | −69.024 | −4.534 | 39.409 | 1.00 | 61.64 | O |
| ATOM | 529 | N | HIS | A | 181 | −69.515 | −6.655 | 40.060 | 1.00 | 52.70 | N |
| ATOM | 530 | CA | HIS | A | 181 | −69.114 | −7.346 | 38.852 | 1.00 | 52.39 | C |
| ATOM | 531 | CB | HIS | A | 181 | −69.260 | −8.870 | 39.064 | 1.00 | 59.89 | C |
| ATOM | 532 | CG | HIS | A | 181 | −68.550 | −9.710 | 38.041 | 1.00 | 66.22 | C |
| ATOM | 533 | ND1 | HIS | A | 181 | −67.178 | −9.846 | 38.012 | 1.00 | 68.32 | N |
| ATOM | 534 | CE1 | HIS | A | 181 | −66.838 | −10.640 | 37.010 | 1.00 | 68.51 | C |
| ATOM | 535 | NE2 | HIS | A | 181 | −67.938 | −11.026 | 36.393 | 1.00 | 70.12 | N |
| ATOM | 536 | CD2 | HIS | A | 181 | −69.025 | −10.464 | 37.021 | 1.00 | 63.07 | C |
| ATOM | 537 | C | HIS | A | 181 | −69.918 | −6.913 | 37.678 | 1.00 | 56.85 | C |
| ATOM | 538 | O | HIS | A | 181 | −69.408 | −6.725 | 36.610 | 1.00 | 60.11 | O |
| ATOM | 539 | N | GLN | A | 182 | −71.206 | −6.767 | 37.896 | 1.00 | 67.47 | N |
| ATOM | 540 | CA | GLN | A | 182 | −72.137 | −6.345 | 36.876 | 1.00 | 60.96 | C |
| ATOM | 541 | CB | GLN | A | 182 | −73.602 | −6.634 | 37.274 | 1.00 | 62.82 | C |
| ATOM | 542 | CG | GLN | A | 182 | −73.942 | −8.122 | 37.479 | 1.00 | 72.09 | C |
| ATOM | 543 | CD | GLN | A | 182 | −75.443 | −8.421 | 37.413 | 1.00 | 80.99 | C |
| ATOM | 544 | OE1 | GLN | A | 182 | −76.265 | −7.523 | 37.187 | 1.00 | 65.51 | O |
| ATOM | 545 | NE2 | GLN | A | 182 | −75.801 | −9.693 | 37.611 | 1.00 | 84.09 | N |
| ATOM | 546 | C | GLN | A | 182 | −71.951 | −4.870 | 36.523 | 1.00 | 56.28 | C |
| ATOM | 547 | O | GLN | A | 182 | −71.979 | −4.490 | 35.357 | 1.00 | 54.55 | O |
| ATOM | 548 | N | SER | A | 183 | −71.864 | −4.029 | 37.564 | 1.00 | 53.74 | N |
| ATOM | 549 | CA | SER | A | 183 | −71.806 | −2.582 | 37.376 | 1.00 | 57.51 | C |
| ATOM | 550 | CB | SER | A | 183 | −71.826 | −1.878 | 38.733 | 1.00 | 67.37 | C |
| ATOM | 551 | OG | SER | A | 183 | −70.695 | −2.236 | 39.503 | 1.00 | 59.93 | O |
| ATOM | 552 | C | SER | A | 183 | −70.606 | −2.081 | 36.583 | 1.00 | 57.85 | C |
| ATOM | 553 | O | SER | A | 183 | −70.660 | −1.044 | 35.938 | 1.00 | 61.48 | O |
| ATOM | 554 | N | ASN | A | 184 | −69.519 | −2.823 | 36.673 | 1.00 | 62.03 | N |
| ATOM | 555 | CA | ASN | A | 184 | −68.282 | −2.506 | 35.992 | 1.00 | 61.71 | C |
| ATOM | 556 | CB | ASN | A | 184 | −68.458 | −2.662 | 34.474 | 1.00 | 64.98 | C |
| ATOM | 557 | CG | ASN | A | 184 | −67.168 | −2.455 | 33.703 | 1.00 | 65.85 | C |
| ATOM | 558 | OD1 | ASN | A | 184 | −66.240 | −3.253 | 33.795 | 1.00 | 71.33 | O |
| ATOM | 559 | ND2 | ASN | A | 184 | −67.112 | −1.381 | 32.925 | 1.00 | 68.57 | N |
| ATOM | 560 | C | ASN | A | 184 | −67.846 | −1.110 | 36.362 | 1.00 | 63.84 | C |
| ATOM | 561 | O | ASN | A | 184 | −67.564 | −0.287 | 35.520 | 1.00 | 63.83 | O |
| ATOM | 562 | N | VAL | A | 185 | −67.723 | −0.882 | 37.650 | 1.00 | 68.81 | N |
| ATOM | 563 | CA | VAL | A | 185 | −67.252 | 0.396 | 38.154 | 1.00 | 63.39 | C |
| ATOM | 564 | CB | VAL | A | 185 | −67.420 | 0.475 | 39.706 | 1.00 | 63.51 | C |
| ATOM | 565 | CG1 | VAL | A | 185 | −67.020 | 1.838 | 40.258 | 1.00 | 59.22 | C |
| ATOM | 566 | CG2 | VAL | A | 185 | −68.857 | 0.174 | 40.099 | 1.00 | 75.50 | C |
| ATOM | 567 | C | VAL | A | 185 | −65.804 | 0.628 | 37.755 | 1.00 | 68.85 | C |
| ATOM | 568 | O | VAL | A | 185 | −65.089 | −0.304 | 37.423 | 1.00 | 73.11 | O |
| ATOM | 569 | O | GLY | A | 186 | −62.072 | 0.948 | 37.666 | 1.00 | 61.61 | O |
| ATOM | 570 | N | GLY | A | 186 | −65.368 | 1.878 | 37.783 | 1.00 | 68.58 | N |
| ATOM | 571 | CA | GLY | A | 186 | −64.030 | 2.242 | 37.389 | 1.00 | 69.98 | C |
| ATOM | 572 | C | GLY | A | 186 | −62.975 | 1.541 | 38.222 | 1.00 | 63.48 | C |
| ATOM | 573 | O | TRP | A | 187 | −60.932 | −1.102 | 40.348 | 1.00 | 56.57 | O |
| ATOM | 574 | N | TRP | A | 187 | −63.054 | 1.613 | 39.544 | 1.00 | 59.13 | N |
| ATOM | 575 | CA | TRP | A | 187 | −62.058 | 0.983 | 40.393 | 1.00 | 57.24 | C |
| ATOM | 576 | C | TRP | A | 187 | −61.987 | −0.527 | 40.206 | 1.00 | 59.95 | C |
| ATOM | 577 | CB | TRP | A | 187 | −62.250 | 1.366 | 41.862 | 1.00 | 62.71 | C |
| ATOM | 578 | CG | TRP | A | 187 | −63.456 | 0.776 | 42.526 | 1.00 | 69.58 | C |
| ATOM | 579 | CD1 | TRP | A | 187 | −64.664 | 1.376 | 42.711 | 1.00 | 62.90 | C |
| ATOM | 580 | CD2 | TRP | A | 187 | −63.556 | −0.524 | 43.125 | 1.00 | 68.22 | C |
| ATOM | 581 | NE1 | TRP | A | 187 | −65.516 | 0.527 | 43.378 | 1.00 | 62.20 | N |
| ATOM | 582 | CE2 | TRP | A | 187 | −64.859 | −0.645 | 43.644 | 1.00 | 58.11 | C |
| ATOM | 583 | CE3 | TRP | A | 187 | −62.672 | −1.599 | 43.268 | 1.00 | 62.05 | C |
| ATOM | 584 | CZ2 | TRP | A | 187 | −65.299 | −1.796 | 44.293 | 1.00 | 61.22 | C |
| ATOM | 585 | CZ3 | TRP | A | 187 | −63.112 | −2.743 | 43.912 | 1.00 | 53.98 | C |
| ATOM | 586 | CH2 | TRP | A | 187 | −64.412 | −2.831 | 44.417 | 1.00 | 59.84 | C |
| ATOM | 587 | O | CYS | A | 188 | −61.523 | −3.903 | 38.502 | 1.00 | 45.62 | O |
| ATOM | 588 | N | CYS | A | 188 | −63.119 | −1.152 | 39.872 | 1.00 | 61.96 | N |
| ATOM | 589 | CA | CYS | A | 188 | −63.189 | −2.583 | 39.581 | 1.00 | 59.64 | C |
| ATOM | 590 | C | CYS | A | 188 | −62.271 | −2.954 | 38.435 | 1.00 | 55.64 | C |
| ATOM | 591 | CB | CYS | A | 188 | −64.629 | −2.989 | 39.264 | 1.00 | 65.52 | C |
| ATOM | 592 | SG | CYS | A | 188 | −64.921 | −4.771 | 39.333 | 1.00 | 92.91 | S |
| ATOM | 593 | N | ARG | A | 189 | −62.345 | −2.186 | 37.369 | 1.00 | 58.81 | N |
| ATOM | 594 | CA | ARG | A | 189 | −61.501 | −2.405 | 36.215 | 1.00 | 56.10 | C |
| ATOM | 595 | CB | ARG | A | 189 | −61.953 | −1.539 | 35.033 | 1.00 | 57.02 | C |
| ATOM | 596 | CG | ARG | A | 189 | −63.244 | −2.046 | 34.392 | 1.00 | 55.06 | C |
| ATOM | 597 | CD | ARG | A | 189 | −63.771 | −1.112 | 33.315 | 1.00 | 56.91 | C |
| ATOM | 598 | NE | ARG | A | 189 | −64.468 | 0.044 | 33.871 | 1.00 | 61.20 | N |
| ATOM | 599 | CZ | ARG | A | 189 | −65.073 | 0.971 | 33.136 | 1.00 | 62.73 | C |
| ATOM | 600 | NH1 | ARG | A | 189 | −65.065 | 0.876 | 31.813 | 1.00 | 56.49 | N |
| ATOM | 601 | NH2 | ARG | A | 189 | −65.685 | 1.993 | 33.720 | 1.00 | 67.57 | N |
| ATOM | 602 | C | ARG | A | 189 | −60.001 | −2.256 | 36.519 | 1.00 | 46.17 | C |
| ATOM | 603 | O | ARG | A | 189 | −59.205 | −3.065 | 36.094 | 1.00 | 41.44 | O |
| ATOM | 604 | N | LEU | A | 190 | −59.647 | −1.249 | 37.332 | 1.00 | 48.12 | N |
| ATOM | 605 | CA | LEU | A | 190 | −58.268 | −1.044 | 37.824 | 1.00 | 47.61 | C |
| ATOM | 606 | CB | LEU | A | 190 | −58.176 | 0.210 | 38.700 | 1.00 | 47.27 | C |
| ATOM | 607 | CG | LEU | A | 190 | −56.798 | 0.475 | 39.319 | 1.00 | 42.49 | C |
| ATOM | 608 | CD1 | LEU | A | 190 | −55.763 | 0.721 | 38.234 | 1.00 | 48.93 | C |
| ATOM | 609 | CD2 | LEU | A | 190 | −56.833 | 1.643 | 40.291 | 1.00 | 46.76 | C |
| ATOM | 610 | C | LEU | A | 190 | −57.705 | −2.256 | 38.593 | 1.00 | 44.07 | C |
| ATOM | 611 | O | LEU | A | 190 | −56.613 | −2.711 | 38.361 | 1.00 | 47.25 | O |
| ATOM | 612 | N | VAL | A | 191 | −58.471 | −2.788 | 39.515 | 1.00 | 44.33 | N |
| ATOM | 613 | CA | VAL | A | 191 | −58.097 | −3.974 | 40.240 | 1.00 | 39.99 | C |
| ATOM | 614 | CB | VAL | A | 191 | −59.149 | −4.345 | 41.312 | 1.00 | 42.50 | C |
| ATOM | 615 | CG1 | VAL | A | 191 | −58.744 | −5.617 | 42.054 | 1.00 | 39.91 | C |
| ATOM | 616 | CG2 | VAL | A | 191 | −59.330 | −3.196 | 42.283 | 1.00 | 40.33 | C |
| ATOM | 617 | C | VAL | A | 191 | −57.823 | −5.194 | 39.344 | 1.00 | 42.81 | C |
| ATOM | 618 | O | VAL | A | 191 | −56.836 | −5.875 | 39.485 | 1.00 | 42.62 | O |
| ATOM | 619 | N | THR | A | 192 | −58.692 | −5.442 | 38.384 | 1.00 | 44.89 | N |
| ATOM | 620 | CA | THR | A | 192 | −58.517 | −6.529 | 37.422 | 1.00 | 42.93 | C |
| ATOM | 621 | CB | THR | A | 192 | −59.732 | −6.680 | 36.493 | 1.00 | 45.77 | C |
| ATOM | 622 | OG1 | THR | A | 192 | −60.936 | −6.668 | 37.270 | 1.00 | 47.11 | O |
| ATOM | 623 | CG2 | THR | A | 192 | −59.646 | −7.986 | 35.716 | 1.00 | 43.43 | C |
| ATOM | 624 | C | THR | A | 192 | −57.243 | −6.367 | 36.596 | 1.00 | 41.20 | C |
| ATOM | 625 | O | THR | A | 192 | −56.454 | −7.272 | 36.462 | 1.00 | 41.34 | O |
| ATOM | 626 | N | ALA | A | 193 | −57.032 | −5.163 | 36.086 | 1.00 | 41.78 | N |
| ATOM | 627 | CA | ALA | A | 193 | −55.823 | −4.825 | 35.354 | 1.00 | 42.55 | C |
| ATOM | 628 | CB | ALA | A | 193 | −55.935 | −3.418 | 34.777 | 1.00 | 35.27 | C |
| ATOM | 629 | C | ALA | A | 193 | −54.576 | −4.955 | 36.210 | 1.00 | 43.64 | C |
| ATOM | 630 | O | ALA | A | 193 | −53.606 | −5.546 | 35.818 | 1.00 | 40.09 | O |
| ATOM | 631 | N | ALA | A | 194 | −54.637 | −4.443 | 37.422 | 1.00 | 43.27 | N |
| ATOM | 632 | CA | ALA | A | 194 | −53.533 | −4.533 | 38.353 | 1.00 | 36.70 | C |
| ATOM | 633 | CB | ALA | A | 194 | −53.855 | −3.777 | 39.632 | 1.00 | 27.27 | C |
| ATOM | 634 | C | ALA | A | 194 | −53.170 | −5.959 | 38.663 | 1.00 | 41.00 | C |
| ATOM | 635 | O | ALA | A | 194 | −52.042 | −6.327 | 38.636 | 1.00 | 38.22 | O |
| ATOM | 636 | N | TYR | A | 195 | −54.159 | −6.776 | 38.912 | 1.00 | 47.92 | N |
| ATOM | 637 | CA | TYR | A | 195 | −53.960 | −8.160 | 39.248 | 1.00 | 45.61 | C |
| ATOM | 638 | CB | TYR | A | 195 | −55.314 | −8.766 | 39.636 | 1.00 | 34.31 | C |
| ATOM | 639 | CG | TYR | A | 195 | −55.278 | −10.215 | 40.054 | 1.00 | 35.29 | C |
| ATOM | 640 | CD1 | TYR | A | 195 | −54.690 | −10.609 | 41.252 | 1.00 | 36.64 | C |
| ATOM | 641 | CE1 | TYR | A | 195 | −54.666 | −11.942 | 41.629 | 1.00 | 40.15 | C |
| ATOM | 642 | CZ | TYR | A | 195 | −55.241 | −12.887 | 40.805 | 1.00 | 38.50 | C |
| ATOM | 643 | OH | TYR | A | 195 | −55.233 | −14.217 | 41.151 | 1.00 | 43.41 | O |
| ATOM | 644 | CE2 | TYR | A | 195 | −55.832 | −12.512 | 39.623 | 1.00 | 37.48 | C |
| ATOM | 645 | CD2 | TYR | A | 195 | −55.852 | −11.188 | 39.257 | 1.00 | 38.89 | C |
| ATOM | 646 | C | TYR | A | 195 | −53.342 | −8.916 | 38.075 | 1.00 | 45.54 | C |
| ATOM | 647 | O | TYR | A | 195 | −52.433 | −9.694 | 38.230 | 1.00 | 45.13 | O |
| ATOM | 648 | N | ASN | A | 196 | −53.811 | −8.626 | 36.881 | 1.00 | 43.86 | N |
| ATOM | 649 | CA | ASN | A | 196 | −53.221 | −9.184 | 35.678 | 1.00 | 46.36 | C |
| ATOM | 650 | CB | ASN | A | 196 | −54.105 | −8.892 | 34.464 | 1.00 | 49.33 | C |
| ATOM | 651 | CG | ASN | A | 196 | −55.387 | −9.699 | 34.480 | 1.00 | 43.06 | C |
| ATOM | 652 | OD1 | ASN | A | 196 | −55.423 | −10.812 | 35.009 | 1.00 | 44.12 | O |
| ATOM | 653 | ND2 | ASN | A | 196 | −56.444 | −9.147 | 33.897 | 1.00 | 46.76 | N |
| ATOM | 654 | C | ASN | A | 196 | −51.794 | −8.709 | 35.437 | 1.00 | 45.38 | C |
| ATOM | 655 | O | ASN | A | 196 | −50.951 | −9.489 | 35.073 | 1.00 | 44.75 | O |
| ATOM | 656 | N | TYR | A | 197 | −51.526 | −7.425 | 35.661 | 1.00 | 43.18 | N |
| ATOM | 657 | CA | TYR | A | 197 | −50.174 | −6.872 | 35.533 | 1.00 | 43.38 | C |
| ATOM | 658 | CB | TYR | A | 197 | −50.127 | −5.391 | 35.928 | 1.00 | 43.04 | C |
| ATOM | 659 | CG | TYR | A | 197 | −48.711 | −4.858 | 36.041 | 1.00 | 47.86 | C |
| ATOM | 660 | CD1 | TYR | A | 197 | −47.939 | −4.607 | 34.906 | 1.00 | 47.64 | C |
| ATOM | 661 | CE1 | TYR | A | 197 | −46.633 | −4.135 | 35.012 | 1.00 | 45.86 | C |
| ATOM | 662 | CZ | TYR | A | 197 | −46.092 | −3.912 | 36.260 | 1.00 | 48.77 | C |
| ATOM | 663 | OH | TYR | A | 197 | −44.805 | −3.441 | 36.382 | 1.00 | 51.73 | O |
| ATOM | 664 | CE2 | TYR | A | 197 | −46.837 | −4.158 | 37.396 | 1.00 | 52.35 | C |
| ATOM | 665 | CD2 | TYR | A | 197 | −48.135 | −4.628 | 37.283 | 1.00 | 52.82 | C |
| ATOM | 666 | C | TYR | A | 197 | −49.142 | −7.643 | 36.338 | 1.00 | 44.88 | C |
| ATOM | 667 | O | TYR | A | 197 | −48.184 | −8.135 | 35.786 | 1.00 | 44.84 | O |
| ATOM | 668 | N | PHE | A | 198 | −49.458 | −7.849 | 37.615 | 1.00 | 45.07 | N |
| ATOM | 669 | CA | PHE | A | 198 | −48.673 | −8.645 | 38.533 | 1.00 | 44.75 | C |
| ATOM | 670 | CB | PHE | A | 198 | −49.255 | −8.551 | 39.944 | 1.00 | 44.19 | C |
| ATOM | 671 | CG | PHE | A | 198 | −49.252 | −7.161 | 40.505 | 1.00 | 49.83 | C |
| ATOM | 672 | CD1 | PHE | A | 198 | −48.155 | −6.332 | 40.331 | 1.00 | 47.41 | C |
| ATOM | 673 | CE1 | PHE | A | 198 | −48.150 | −5.049 | 40.843 | 1.00 | 46.60 | C |
| ATOM | 674 | CZ | PHE | A | 198 | −49.248 | −4.578 | 41.536 | 1.00 | 48.47 | C |
| ATOM | 675 | CE2 | PHE | A | 198 | −50.349 | −5.391 | 41.712 | 1.00 | 48.43 | C |
| ATOM | 676 | CD2 | PHE | A | 198 | −50.348 | −6.676 | 41.198 | 1.00 | 49.97 | C |
| ATOM | 677 | C | PHE | A | 198 | −48.548 | −10.114 | 38.123 | 1.00 | 44.04 | C |
| ATOM | 678 | O | PHE | A | 198 | −47.629 | −10.802 | 38.507 | 1.00 | 41.85 | O |
| ATOM | 679 | N | HIS | A | 199 | −49.455 | −10.574 | 37.283 | 1.00 | 43.21 | N |
| ATOM | 680 | CA | HIS | A | 199 | −49.416 | −11.918 | 36.792 | 1.00 | 44.90 | C |
| ATOM | 681 | CB | HIS | A | 199 | −50.758 | −12.342 | 36.205 | 1.00 | 45.51 | C |
| ATOM | 682 | CG | HIS | A | 199 | −50.920 | −13.825 | 36.127 | 1.00 | 47.81 | C |
| ATOM | 683 | ND1 | HIS | A | 199 | −51.977 | −14.429 | 35.481 | 1.00 | 51.90 | N |
| ATOM | 684 | CE1 | HIS | A | 199 | −51.851 | −15.740 | 35.574 | 1.00 | 55.70 | C |
| ATOM | 685 | NE2 | HIS | A | 199 | −50.752 | −16.009 | 36.258 | 1.00 | 45.59 | N |
| ATOM | 686 | CD2 | HIS | A | 199 | −50.152 | −14.826 | 36.614 | 1.00 | 45.67 | C |
| ATOM | 687 | C | HIS | A | 199 | −48.323 | −12.083 | 35.773 | 1.00 | 46.03 | C |
| ATOM | 688 | O | HIS | A | 199 | −47.588 | −13.042 | 35.758 | 1.00 | 43.48 | O |
| ATOM | 689 | N | VAL | A | 200 | −48.202 | −11.068 | 34.939 | 1.00 | 42.12 | N |
| ATOM | 690 | CA | VAL | A | 200 | −47.184 | −11.016 | 33.939 | 1.00 | 41.94 | C |
| ATOM | 691 | CB | VAL | A | 200 | −47.502 | −9.919 | 32.910 | 1.00 | 42.18 | C |
| ATOM | 692 | CG1 | VAL | A | 200 | −46.635 | −10.072 | 31.682 | 1.00 | 42.25 | C |
| ATOM | 693 | CG2 | VAL | A | 200 | −48.965 | −9.990 | 32.519 | 1.00 | 45.75 | C |
| ATOM | 694 | C | VAL | A | 200 | −45.832 | −10.758 | 34.589 | 1.00 | 44.86 | C |
| ATOM | 695 | O | VAL | A | 200 | −44.824 | −11.269 | 34.179 | 1.00 | 42.76 | O |
| ATOM | 696 | N | THR | A | 201 | −45.841 | −9.972 | 35.648 | 1.00 | 42.73 | N |
| ATOM | 697 | CA | THR | A | 201 | −44.673 | −9.627 | 36.419 | 1.00 | 39.15 | C |
| ATOM | 698 | CB | THR | A | 201 | −45.011 | −8.617 | 37.529 | 1.00 | 41.70 | C |
| ATOM | 699 | OG1 | THR | A | 201 | −45.728 | −7.512 | 36.965 | 1.00 | 47.81 | O |
| ATOM | 700 | CG2 | THR | A | 201 | −43.746 | −8.097 | 38.165 | 1.00 | 50.62 | C |
| ATOM | 701 | C | THR | A | 201 | −44.021 | −10.845 | 37.006 | 1.00 | 39.26 | C |
| ATOM | 702 | O | THR | A | 201 | −42.831 | −11.008 | 36.948 | 1.00 | 41.88 | O |
| ATOM | 703 | N | ASN | A | 202 | −44.837 | −11.732 | 37.510 | 1.00 | 39.69 | N |
| ATOM | 704 | CA | ASN | A | 202 | −44.413 | −13.030 | 38.003 | 1.00 | 40.51 | C |
| ATOM | 705 | CB | ASN | A | 202 | −45.612 | −13.823 | 38.526 | 1.00 | 41.37 | C |
| ATOM | 706 | CG | ASN | A | 202 | −46.150 | −13.278 | 39.838 | 1.00 | 45.30 | C |
| ATOM | 707 | OD1 | ASN | A | 202 | −45.828 | −12.161 | 40.244 | 1.00 | 43.56 | O |
| ATOM | 708 | ND2 | ASN | A | 202 | −46.977 | −14.072 | 40.509 | 1.00 | 48.58 | N |
| ATOM | 709 | C | ASN | A | 202 | −43.667 | −13.858 | 36.956 | 1.00 | 41.51 | C |
| ATOM | 710 | O | ASN | A | 202 | −42.594 | −14.368 | 37.189 | 1.00 | 40.64 | O |
| ATOM | 711 | N | PHE | A | 203 | −44.268 | −13.997 | 35.791 | 1.00 | 44.31 | N |
| ATOM | 712 | CA | PHE | A | 203 | −43.671 | −14.717 | 34.680 | 1.00 | 41.81 | C |
| ATOM | 713 | CB | PHE | A | 203 | −44.643 | −14.843 | 33.504 | 1.00 | 37.83 | C |
| ATOM | 714 | CG | PHE | A | 203 | −45.515 | −16.065 | 33.584 | 1.00 | 43.45 | C |
| ATOM | 715 | CD2 | PHE | A | 203 | −45.350 | −17.110 | 32.693 | 1.00 | 45.88 | C |
| ATOM | 716 | CE2 | PHE | A | 203 | −46.140 | −18.243 | 32.774 | 1.00 | 45.24 | C |
| ATOM | 717 | CZ | PHE | A | 203 | −47.103 | −18.343 | 33.752 | 1.00 | 46.03 | C |
| ATOM | 718 | CE1 | PHE | A | 203 | −47.275 | −17.311 | 34.651 | 1.00 | 44.81 | C |
| ATOM | 719 | CD1 | PHE | A | 203 | −46.481 | −16.179 | 34.567 | 1.00 | 46.22 | C |
| ATOM | 720 | C | PHE | A | 203 | −42.353 | −14.102 | 34.263 | 1.00 | 42.38 | C |
| ATOM | 721 | O | PHE | A | 203 | −41.367 | −14.773 | 34.125 | 1.00 | 42.00 | O |
| ATOM | 722 | N | PHE | A | 204 | −42.344 | −12.809 | 34.027 | 1.00 | 41.27 | N |
| ATOM | 723 | CA | PHE | A | 204 | −41.147 | −12.120 | 33.587 | 1.00 | 36.52 | C |
| ATOM | 724 | CB | PHE | A | 204 | −41.461 | −10.699 | 33.123 | 1.00 | 35.47 | C |
| ATOM | 725 | CG | PHE | A | 204 | −42.002 | −10.626 | 31.722 | 1.00 | 40.40 | C |
| ATOM | 726 | CD2 | PHE | A | 204 | −41.266 | −10.040 | 30.712 | 1.00 | 40.56 | C |
| ATOM | 727 | CE2 | PHE | A | 204 | −41.767 | −9.968 | 29.425 | 1.00 | 43.69 | C |
| ATOM | 728 | CZ | PHE | A | 204 | −43.012 | −10.483 | 29.135 | 1.00 | 42.88 | C |
| ATOM | 729 | CE1 | PHE | A | 204 | −43.752 | −11.070 | 30.128 | 1.00 | 43.17 | C |
| ATOM | 730 | CD1 | PHE | A | 204 | −43.251 | −11.138 | 31.417 | 1.00 | 46.18 | C |
| ATOM | 731 | C | PHE | A | 204 | −40.027 | −12.124 | 34.630 | 1.00 | 41.98 | C |
| ATOM | 732 | O | PHE | A | 204 | −38.905 | −12.367 | 34.291 | 1.00 | 45.64 | O |
| ATOM | 733 | N | TRP | A | 205 | −40.329 | −11.947 | 35.914 | 1.00 | 44.49 | N |
| ATOM | 734 | CA | TRP | A | 205 | −39.328 | −12.158 | 36.985 | 1.00 | 39.50 | C |
| ATOM | 735 | CB | TRP | A | 205 | −39.817 | −11.658 | 38.351 | 1.00 | 36.98 | C |
| ATOM | 736 | CG | TRP | A | 205 | −39.543 | −10.191 | 38.509 | 1.00 | 39.56 | C |
| ATOM | 737 | CD1 | TRP | A | 205 | −40.463 | −9.184 | 38.576 | 1.00 | 40.17 | C |
| ATOM | 738 | NE1 | TRP | A | 205 | −39.831 | −7.970 | 38.686 | 1.00 | 38.22 | N |
| ATOM | 739 | CE2 | TRP | A | 205 | −38.476 | −8.172 | 38.675 | 1.00 | 44.80 | C |
| ATOM | 740 | CD2 | TRP | A | 205 | −38.255 | −9.559 | 38.557 | 1.00 | 44.15 | C |
| ATOM | 741 | CE3 | TRP | A | 205 | −36.937 | −10.030 | 38.522 | 1.00 | 38.97 | C |
| ATOM | 742 | CZ3 | TRP | A | 205 | −35.899 | −9.112 | 38.605 | 1.00 | 37.69 | C |
| ATOM | 743 | CH2 | TRP | A | 205 | −36.154 | −7.740 | 38.717 | 1.00 | 41.35 | C |
| ATOM | 744 | CZ2 | TRP | A | 205 | −37.432 | −7.253 | 38.752 | 1.00 | 44.21 | C |
| ATOM | 745 | C | TRP | A | 205 | −38.752 | −13.575 | 37.049 | 1.00 | 40.09 | C |
| ATOM | 746 | O | TRP | A | 205 | −37.575 | −13.771 | 37.188 | 1.00 | 45.11 | O |
| ATOM | 747 | N | MET | A | 206 | −39.573 | −14.581 | 36.881 | 1.00 | 44.78 | N |
| ATOM | 748 | CA | MET | A | 206 | −39.074 | −15.951 | 36.769 | 1.00 | 49.88 | C |
| ATOM | 749 | CB | MET | A | 206 | −40.239 | −16.932 | 36.606 | 1.00 | 49.17 | C |
| ATOM | 750 | CG | MET | A | 206 | −40.977 | −17.266 | 37.891 | 1.00 | 44.71 | C |
| ATOM | 751 | SD | MET | A | 206 | −39.953 | −18.196 | 39.049 | 1.00 | 58.12 | S |
| ATOM | 752 | CE | MET | A | 206 | −41.145 | −18.551 | 40.338 | 1.00 | 62.30 | C |
| ATOM | 753 | C | MET | A | 206 | −38.115 | −16.097 | 35.571 | 1.00 | 49.98 | C |
| ATOM | 754 | O | MET | A | 206 | −37.041 | −16.655 | 35.676 | 1.00 | 53.96 | O |
| ATOM | 755 | N | PHE | A | 207 | −38.489 | −15.460 | 34.469 | 1.00 | 45.29 | N |
| ATOM | 756 | CA | PHE | A | 207 | −37.665 | −15.331 | 33.272 | 1.00 | 45.65 | C |
| ATOM | 757 | CB | PHE | A | 207 | −38.463 | −14.634 | 32.175 | 1.00 | 45.14 | C |
| ATOM | 758 | CG | PHE | A | 207 | −37.658 | −14.285 | 30.961 | 1.00 | 47.64 | C |
| ATOM | 759 | CD1 | PHE | A | 207 | −36.946 | −15.254 | 30.277 | 1.00 | 51.52 | C |
| ATOM | 760 | CE1 | PHE | A | 207 | −36.208 | −14.924 | 29.157 | 1.00 | 54.13 | C |
| ATOM | 761 | CZ | PHE | A | 207 | −36.188 | −13.616 | 28.707 | 1.00 | 58.21 | C |
| ATOM | 762 | CE2 | PHE | A | 207 | −36.901 | −12.647 | 29.378 | 1.00 | 52.72 | C |
| ATOM | 763 | CD2 | PHE | A | 207 | −37.633 | −12.984 | 30.493 | 1.00 | 50.75 | C |
| ATOM | 764 | C | PHE | A | 207 | −36.367 | −14.548 | 33.548 | 1.00 | 50.40 | C |
| ATOM | 765 | O | PHE | A | 207 | −35.299 | −14.857 | 33.068 | 1.00 | 54.38 | O |
| ATOM | 766 | N | GLY | A | 208 | −36.458 | −13.598 | 34.438 | 1.00 | 50.61 | N |
| ATOM | 767 | CA | GLY | A | 208 | −35.335 | −12.916 | 35.022 | 1.00 | 50.13 | C |
| ATOM | 768 | C | GLY | A | 208 | −34.341 | −13.867 | 35.614 | 1.00 | 46.82 | C |
| ATOM | 769 | O | GLY | A | 208 | −33.197 | −13.806 | 35.263 | 1.00 | 53.67 | O |
| ATOM | 770 | N | GLU | A | 209 | −34.750 | −14.754 | 36.506 | 1.00 | 42.00 | N |
| ATOM | 771 | CA | GLU | A | 209 | −33.857 | −15.731 | 37.082 | 1.00 | 49.66 | C |
| ATOM | 772 | CB | GLU | A | 209 | −34.625 | −16.593 | 38.084 | 1.00 | 54.36 | C |
| ATOM | 773 | CG | GLU | A | 209 | −35.127 | −15.848 | 39.298 | 1.00 | 60.51 | C |
| ATOM | 774 | CD | GLU | A | 209 | −34.069 | −15.710 | 40.373 | 1.00 | 67.72 | C |
| ATOM | 775 | OE1 | GLU | A | 209 | −32.975 | −16.300 | 40.226 | 1.00 | 67.06 | O |
| ATOM | 776 | OE2 | GLU | A | 209 | −34.338 | −15.004 | 41.365 | 1.00 | 64.88 | O |
| ATOM | 777 | C | GLU | A | 209 | −33.239 | −16.643 | 36.051 | 1.00 | 49.03 | C |
| ATOM | 778 | O | GLU | A | 209 | −32.139 | −17.128 | 36.223 | 1.00 | 49.69 | O |
| ATOM | 779 | N | GLY | A | 210 | −33.998 | −16.959 | 35.025 | 1.00 | 46.48 | N |
| ATOM | 780 | CA | GLY | A | 210 | −33.489 | −17.789 | 33.980 | 1.00 | 45.16 | C |
| ATOM | 781 | C | GLY | A | 210 | −32.498 | −17.193 | 33.060 | 1.00 | 51.13 | C |
| ATOM | 782 | O | GLY | A | 210 | −31.684 | −17.908 | 32.505 | 1.00 | 52.96 | O |
| ATOM | 783 | N | CYS | A | 211 | −32.587 | −15.884 | 32.839 | 1.00 | 55.65 | N |
| ATOM | 784 | CA | CYS | A | 211 | −31.576 | −15.177 | 32.073 | 1.00 | 53.67 | C |
| ATOM | 785 | CB | CYS | A | 211 | −32.017 | −13.735 | 31.789 | 1.00 | 51.58 | C |
| ATOM | 786 | SG | CYS | A | 211 | −33.516 | −13.530 | 30.810 | 1.00 | 58.43 | S |
| ATOM | 787 | C | CYS | A | 211 | −30.282 | −15.118 | 32.835 | 1.00 | 53.03 | C |
| ATOM | 788 | O | CYS | A | 211 | −29.229 | −15.337 | 32.275 | 1.00 | 53.83 | O |
| ATOM | 789 | N | TYR | A | 212 | −30.388 | −14.816 | 34.131 | 1.00 | 52.55 | N |
| ATOM | 790 | CA | TYR | A | 212 | −29.246 | −14.766 | 35.015 | 1.00 | 48.57 | C |
| ATOM | 791 | CB | TYR | A | 212 | −29.672 | −14.317 | 36.420 | 1.00 | 46.86 | C |
| ATOM | 792 | CG | TYR | A | 212 | −28.586 | −14.523 | 37.459 | 1.00 | 52.62 | C |
| ATOM | 793 | CD1 | TYR | A | 212 | −27.570 | −13.590 | 37.631 | 1.00 | 45.52 | C |
| ATOM | 794 | CE1 | TYR | A | 212 | −26.563 | −13.790 | 38.564 | 1.00 | 47.35 | C |
| ATOM | 795 | CZ | TYR | A | 212 | −26.561 | −14.933 | 39.330 | 1.00 | 51.64 | C |
| ATOM | 796 | OH | TYR | A | 212 | −25.558 | −15.141 | 40.251 | 1.00 | 58.11 | O |
| ATOM | 797 | CE2 | TYR | A | 212 | −27.553 | −15.876 | 39.173 | 1.00 | 50.91 | C |
| ATOM | 798 | CD2 | TYR | A | 212 | −28.558 | −15.668 | 38.245 | 1.00 | 52.37 | C |
| ATOM | 799 | C | TYR | A | 212 | −28.520 | −16.100 | 35.099 | 1.00 | 54.74 | C |
| ATOM | 800 | O | TYR | A | 212 | −27.315 | −16.158 | 35.009 | 1.00 | 59.96 | O |
| ATOM | 801 | N | LEU | A | 213 | −29.255 | −17.177 | 35.302 | 1.00 | 56.47 | N |
| ATOM | 802 | CA | LEU | A | 213 | −28.636 | −18.473 | 35.459 | 1.00 | 55.14 | C |
| ATOM | 803 | CB | LEU | A | 213 | −29.695 | −19.514 | 35.820 | 1.00 | 57.65 | C |
| ATOM | 804 | CG | LEU | A | 213 | −29.152 | −20.831 | 36.369 | 1.00 | 67.25 | C |
| ATOM | 805 | CD1 | LEU | A | 213 | −28.267 | −20.571 | 37.587 | 1.00 | 69.15 | C |
| ATOM | 806 | CD2 | LEU | A | 213 | −30.295 | −21.779 | 36.708 | 1.00 | 58.07 | C |
| ATOM | 807 | C | LEU | A | 213 | −27.885 | −18.900 | 34.218 | 1.00 | 56.66 | C |
| ATOM | 808 | O | LEU | A | 213 | −26.773 | −19.383 | 34.273 | 1.00 | 59.34 | O |
| ATOM | 809 | N | HIS | A | 214 | −28.518 | −18.688 | 33.083 | 1.00 | 58.50 | N |
| ATOM | 810 | CA | HIS | A | 214 | −27.948 | −19.018 | 31.795 | 1.00 | 57.26 | C |
| ATOM | 811 | CB | HIS | A | 214 | −28.998 | −18.757 | 30.712 | 1.00 | 60.25 | C |
| ATOM | 812 | CG | HIS | A | 214 | −28.511 | −19.019 | 29.324 | 1.00 | 59.23 | C |
| ATOM | 813 | ND1 | HIS | A | 214 | −27.765 | −18.104 | 28.616 | 1.00 | 59.79 | N |
| ATOM | 814 | CE1 | HIS | A | 214 | −27.474 | −18.606 | 27.428 | 1.00 | 66.41 | C |
| ATOM | 815 | NE2 | HIS | A | 214 | −28.008 | −19.809 | 27.343 | 1.00 | 76.25 | N |
| ATOM | 816 | CD2 | HIS | A | 214 | −28.664 | −20.093 | 28.518 | 1.00 | 61.92 | C |
| ATOM | 817 | C | HIS | A | 214 | −26.669 | −18.240 | 31.494 | 1.00 | 56.23 | C |
| ATOM | 818 | O | HIS | A | 214 | −25.671 | −18.815 | 31.107 | 1.00 | 61.95 | O |
| ATOM | 819 | N | THR | A | 215 | −26.711 | −16.937 | 31.756 | 1.00 | 54.63 | N |
| ATOM | 820 | CA | THR | A | 215 | −25.576 | −16.042 | 31.600 | 1.00 | 53.07 | C |
| ATOM | 821 | CB | THR | A | 215 | −25.954 | −14.547 | 31.648 | 1.00 | 52.71 | C |
| ATOM | 822 | OG1 | THR | A | 215 | −26.607 | −14.248 | 32.888 | 1.00 | 70.28 | O |
| ATOM | 823 | CG2 | THR | A | 215 | −26.866 | −14.187 | 30.478 | 1.00 | 51.66 | C |
| ATOM | 824 | C | THR | A | 215 | −24.491 | −16.362 | 32.618 | 1.00 | 55.71 | C |
| ATOM | 825 | O | THR | A | 215 | −23.321 | −16.196 | 32.371 | 1.00 | 66.36 | O |
| ATOM | 826 | N | ALA | A | 216 | −24.899 | −16.792 | 33.791 | 1.00 | 52.94 | N |
| ATOM | 827 | CA | ALA | A | 216 | −23.991 | −17.128 | 34.867 | 1.00 | 59.88 | C |
| ATOM | 828 | CB | ALA | A | 216 | −24.759 | −17.502 | 36.128 | 1.00 | 61.71 | C |
| ATOM | 829 | C | ALA | A | 216 | −23.040 | −18.251 | 34.470 | 1.00 | 56.08 | C |
| ATOM | 830 | O | ALA | A | 216 | −21.863 | −18.223 | 34.776 | 1.00 | 63.68 | O |
| ATOM | 831 | N | ILE | A | 217 | −23.568 | −19.263 | 33.814 | 1.00 | 55.26 | N |
| ATOM | 832 | CA | ILE | A | 217 | −22.745 | −20.366 | 33.372 | 1.00 | 63.43 | C |
| ATOM | 833 | CB | ILE | A | 217 | −23.583 | −21.611 | 33.098 | 1.00 | 58.34 | C |
| ATOM | 834 | CG1 | ILE | A | 217 | −24.199 | −22.109 | 34.404 | 1.00 | 54.86 | C |
| ATOM | 835 | CD1 | ILE | A | 217 | −24.856 | −23.457 | 34.289 | 1.00 | 64.41 | C |
| ATOM | 836 | CG2 | ILE | A | 217 | −22.724 | −22.684 | 32.475 | 1.00 | 68.47 | C |
| ATOM | 837 | C | ILE | A | 217 | −21.918 | −19.993 | 32.118 | 1.00 | 70.69 | C |
| ATOM | 838 | O | ILE | A | 217 | −20.702 | −20.140 | 32.103 | 1.00 | 77.09 | O |
| ATOM | 839 | N | VAL | A | 218 | −22.578 | −19.526 | 31.063 | 1.00 | 65.20 | N |
| ATOM | 840 | CA | VAL | A | 218 | −21.893 | −19.166 | 29.822 | 1.00 | 65.13 | C |
| ATOM | 841 | CB | VAL | A | 218 | −22.924 | −18.710 | 28.757 | 1.00 | 68.23 | C |
| ATOM | 842 | CG1 | VAL | A | 218 | −22.235 | −18.249 | 27.474 | 1.00 | 73.53 | C |
| ATOM | 843 | CG2 | VAL | A | 218 | −23.913 | −19.829 | 28.467 | 1.00 | 73.40 | C |
| ATOM | 844 | C | VAL | A | 218 | −20.806 | −18.047 | 29.972 | 1.00 | 63.42 | C |
| ATOM | 845 | O | VAL | A | 218 | −19.777 | −18.111 | 29.322 | 1.00 | 69.77 | O |
| ATOM | 846 | N | LEU | A | 219 | −21.070 | −16.999 | 30.763 | 1.00 | 55.70 | N |
| ATOM | 847 | CA | LEU | A | 219 | −20.168 | −15.852 | 30.875 | 1.00 | 56.64 | C |
| ATOM | 848 | CB | LEU | A | 219 | −20.848 | −14.578 | 30.363 | 1.00 | 50.74 | C |
| ATOM | 849 | CG | LEU | A | 219 | −21.334 | −14.572 | 28.912 | 1.00 | 50.38 | C |
| ATOM | 850 | CD1 | LEU | A | 219 | −21.850 | −13.197 | 28.528 | 1.00 | 41.38 | C |
| ATOM | 851 | CD2 | LEU | A | 219 | −20.236 | −15.011 | 27.963 | 1.00 | 52.82 | C |
| ATOM | 852 | C | LEU | A | 219 | −19.654 | −15.629 | 32.306 | 1.00 | 57.48 | C |
| ATOM | 853 | O | LEU | A | 219 | −20.418 | −15.523 | 33.248 | 1.00 | 55.70 | O |
| ATOM | 854 | N | THR | A | 220 | −18.331 | −15.502 | 32.429 | 1.00 | 58.26 | N |
| ATOM | 855 | CA | THR | A | 220 | −17.645 | −15.203 | 33.690 | 1.00 | 57.14 | C |
| ATOM | 856 | CB | THR | A | 220 | −16.127 | −15.483 | 33.587 | 1.00 | 52.40 | C |
| ATOM | 857 | OG1 | THR | A | 220 | −15.542 | −14.635 | 32.589 | 1.00 | 49.86 | O |
| ATOM | 858 | CG2 | THR | A | 220 | −15.874 | −16.939 | 33.221 | 1.00 | 53.52 | C |
| ATOM | 859 | C | THR | A | 220 | −17.852 | −13.761 | 34.180 | 1.00 | 54.78 | C |
| ATOM | 860 | O | THR | A | 220 | −18.179 | −12.864 | 33.429 | 1.00 | 51.19 | O |
| ATOM | 861 | N | ASN | A | 1002 | −17.640 | −13.574 | 35.479 | 1.00 | 56.49 | N |
| ATOM | 862 | CA | ASN | A | 1002 | −17.754 | −12.284 | 36.160 | 1.00 | 51.29 | C |
| ATOM | 863 | CB | ASN | A | 1002 | −17.509 | −12.451 | 37.657 | 1.00 | 58.29 | C |
| ATOM | 864 | CG | ASN | A | 1002 | −18.543 | −13.329 | 38.317 | 1.00 | 58.93 | C |
| ATOM | 865 | OD1 | ASN | A | 1002 | −18.307 | −14.516 | 38.558 | 1.00 | 60.41 | O |
| ATOM | 866 | ND2 | ASN | A | 1002 | −19.703 | −12.751 | 38.614 | 1.00 | 46.97 | N |
| ATOM | 867 | C | ASN | A | 1002 | −16.803 | −11.251 | 35.616 | 1.00 | 46.14 | C |
| ATOM | 868 | O | ASN | A | 1002 | −17.179 | −10.134 | 35.368 | 1.00 | 46.85 | O |
| ATOM | 869 | N | ILE | A | 1003 | −15.559 | −11.646 | 35.430 | 1.00 | 54.90 | N |
| ATOM | 870 | CA | ILE | A | 1003 | −14.533 | −10.766 | 34.910 | 1.00 | 48.77 | C |
| ATOM | 871 | CB | ILE | A | 1003 | −13.111 | −11.365 | 35.019 | 1.00 | 35.18 | C |
| ATOM | 872 | CG1 | ILE | A | 1003 | −12.070 | −10.284 | 34.735 | 1.00 | 34.52 | C |
| ATOM | 873 | CD1 | ILE | A | 1003 | −10.681 | −10.643 | 35.183 | 1.00 | 35.58 | C |
| ATOM | 874 | CG2 | ILE | A | 1003 | −12.942 | −12.558 | 34.085 | 1.00 | 43.87 | C |
| ATOM | 875 | C | ILE | A | 1003 | −14.863 | −10.325 | 33.473 | 1.00 | 45.09 | C |
| ATOM | 876 | O | ILE | A | 1003 | −14.637 | −9.192 | 33.104 | 1.00 | 42.18 | O |
| ATOM | 877 | N | PHE | A | 1004 | −15.508 | −11.198 | 32.691 | 1.00 | 43.36 | N |
| ATOM | 878 | CA | PHE | A | 1004 | −16.008 | −10.800 | 31.384 | 1.00 | 47.51 | C |
| ATOM | 879 | CB | PHE | A | 1004 | −16.639 | −11.979 | 30.643 | 1.00 | 52.14 | C |
| ATOM | 880 | CG | PHE | A | 1004 | −17.102 | −11.641 | 29.251 | 1.00 | 55.17 | C |
| ATOM | 881 | CD2 | PHE | A | 1004 | −16.280 | −11.873 | 28.159 | 1.00 | 57.77 | C |
| ATOM | 882 | CE2 | PHE | A | 1004 | −16.706 | −11.573 | 26.883 | 1.00 | 57.87 | C |
| ATOM | 883 | CZ | PHE | A | 1004 | −17.964 | −11.022 | 26.685 | 1.00 | 54.22 | C |
| ATOM | 884 | CE1 | PHE | A | 1004 | −18.789 | −10.783 | 27.763 | 1.00 | 48.36 | C |
| ATOM | 885 | CD1 | PHE | A | 1004 | −18.360 | −11.095 | 29.034 | 1.00 | 49.45 | C |
| ATOM | 886 | C | PHE | A | 1004 | −17.007 | −9.665 | 31.496 | 1.00 | 49.39 | C |
| ATOM | 887 | O | PHE | A | 1004 | −16.881 | −8.660 | 30.832 | 1.00 | 50.36 | O |
| ATOM | 888 | N | GLU | A | 1005 | −18.007 | −9.835 | 32.353 | 1.00 | 46.51 | N |
| ATOM | 889 | CA | GLU | A | 1005 | −19.030 | −8.815 | 32.544 | 1.00 | 41.43 | C |
| ATOM | 890 | CB | GLU | A | 1005 | −20.110 | −9.334 | 33.494 | 1.00 | 45.57 | C |
| ATOM | 891 | CG | GLU | A | 1005 | −20.845 | −10.563 | 32.984 | 1.00 | 55.74 | C |
| ATOM | 892 | CD | GLU | A | 1005 | −21.719 | −10.269 | 31.775 | 1.00 | 62.50 | C |
| ATOM | 893 | OE1 | GLU | A | 1005 | −22.052 | −9.085 | 31.548 | 1.00 | 60.51 | O |
| ATOM | 894 | OE2 | GLU | A | 1005 | −22.074 | −11.225 | 31.051 | 1.00 | 60.84 | O |
| ATOM | 895 | C | GLU | A | 1005 | −18.495 | −7.484 | 33.060 | 1.00 | 45.25 | C |
| ATOM | 896 | O | GLU | A | 1005 | −18.905 | −6.428 | 32.630 | 1.00 | 47.69 | O |
| ATOM | 897 | N | MET | A | 1006 | −17.543 | −7.534 | 33.971 | 1.00 | 48.73 | N |
| ATOM | 898 | CA | MET | A | 1006 | −16.946 | −6.325 | 34.489 | 1.00 | 45.13 | C |
| ATOM | 899 | CB | MET | A | 1006 | −15.908 | −6.652 | 35.571 | 1.00 | 42.14 | C |
| ATOM | 900 | CG | MET | A | 1006 | −15.052 | −5.450 | 35.980 | 1.00 | 46.38 | C |
| ATOM | 901 | SD | MET | A | 1006 | −13.903 | −5.714 | 37.351 | 1.00 | 38.23 | S |
| ATOM | 902 | CE | MET | A | 1006 | −12.959 | −7.104 | 36.740 | 1.00 | 36.09 | C |
| ATOM | 903 | C | MET | A | 1006 | −16.296 | −5.482 | 33.400 | 1.00 | 42.73 | C |
| ATOM | 904 | O | MET | A | 1006 | −16.438 | −4.281 | 33.361 | 1.00 | 44.39 | O |
| ATOM | 905 | N | LEU | A | 1007 | −15.525 | −6.115 | 32.544 | 1.00 | 39.74 | N |
| ATOM | 906 | CA | LEU | A | 1007 | −14.830 | −5.388 | 31.524 | 1.00 | 46.82 | C |
| ATOM | 907 | CB | LEU | A | 1007 | −13.514 | −6.084 | 31.195 | 1.00 | 46.45 | C |
| ATOM | 908 | CG | LEU | A | 1007 | −12.313 | −5.722 | 32.063 | 1.00 | 44.40 | C |
| ATOM | 909 | CD1 | LEU | A | 1007 | −12.592 | −5.912 | 33.522 | 1.00 | 50.70 | C |
| ATOM | 910 | CD2 | LEU | A | 1007 | −11.229 | −6.641 | 31.699 | 1.00 | 47.95 | C |
| ATOM | 911 | C | LEU | A | 1007 | −15.627 | −5.221 | 30.264 | 1.00 | 51.65 | C |
| ATOM | 912 | O | LEU | A | 1007 | −15.073 | −4.903 | 29.241 | 1.00 | 58.13 | O |
| ATOM | 913 | N | ARG | A | 1008 | −16.925 | −5.435 | 30.333 | 1.00 | 53.51 | N |
| ATOM | 914 | CA | ARG | A | 1008 | −17.794 | −5.227 | 29.189 | 1.00 | 47.83 | C |
| ATOM | 915 | CB | ARG | A | 1008 | −18.750 | −6.415 | 29.026 | 1.00 | 50.16 | C |
| ATOM | 916 | CG | ARG | A | 1008 | −19.775 | −6.254 | 27.907 | 1.00 | 54.48 | C |
| ATOM | 917 | CD | ARG | A | 1008 | −20.988 | −7.163 | 28.084 | 1.00 | 58.89 | C |
| ATOM | 918 | NE | ARG | A | 1008 | −21.663 | −6.965 | 29.365 | 1.00 | 54.95 | N |
| ATOM | 919 | CZ | ARG | A | 1008 | −22.461 | −5.938 | 29.642 | 1.00 | 59.73 | C |
| ATOM | 920 | NH1 | ARG | A | 1008 | −22.676 | −4.991 | 28.734 | 1.00 | 57.02 | N |
| ATOM | 921 | NH2 | ARG | A | 1008 | −23.039 | −5.851 | 30.833 | 1.00 | 61.22 | N |
| ATOM | 922 | C | ARG | A | 1008 | −18.588 | −3.981 | 29.447 | 1.00 | 52.97 | C |
| ATOM | 923 | O | ARG | A | 1008 | −19.089 | −3.321 | 28.546 | 1.00 | 62.43 | O |
| ATOM | 924 | N | ILE | A | 1009 | −18.630 | −3.634 | 30.723 | 1.00 | 53.00 | N |
| ATOM | 925 | CA | ILE | A | 1009 | −19.263 | −2.441 | 31.164 | 1.00 | 50.23 | C |
| ATOM | 926 | CB | ILE | A | 1009 | −19.750 | −2.581 | 32.624 | 1.00 | 49.70 | C |
| ATOM | 927 | CG1 | ILE | A | 1009 | −20.734 | −3.745 | 32.751 | 1.00 | 40.15 | C |
| ATOM | 928 | CD1 | ILE | A | 1009 | −21.272 | −3.928 | 34.144 | 1.00 | 37.08 | C |
| ATOM | 929 | CG2 | ILE | A | 1009 | −20.379 | −1.283 | 33.114 | 1.00 | 48.73 | C |
| ATOM | 930 | C | ILE | A | 1009 | −18.244 | −1.315 | 31.097 | 1.00 | 43.77 | C |
| ATOM | 931 | O | ILE | A | 1009 | −18.570 | −0.199 | 30.729 | 1.00 | 40.26 | O |
| ATOM | 932 | N | ASP | A | 1010 | −17.002 | −1.652 | 31.455 | 1.00 | 44.04 | N |
| ATOM | 933 | CA | ASP | A | 1010 | −15.919 | −0.693 | 31.559 | 1.00 | 42.21 | C |
| ATOM | 934 | CB | ASP | A | 1010 | −14.865 | −1.188 | 32.550 | 1.00 | 41.17 | C |
| ATOM | 935 | CG | ASP | A | 1010 | −15.315 | −1.088 | 33.992 | 1.00 | 40.19 | C |
| ATOM | 936 | OD1 | ASP | A | 1010 | −16.214 | −0.276 | 34.291 | 1.00 | 44.96 | O |
| ATOM | 937 | OD2 | ASP | A | 1010 | −14.748 | −1.822 | 34.829 | 1.00 | 31.80 | O |
| ATOM | 938 | C | ASP | A | 1010 | −15.194 | −0.335 | 30.236 | 1.00 | 39.21 | C |
| ATOM | 939 | O | ASP | A | 1010 | −14.985 | 0.833 | 29.985 | 1.00 | 40.74 | O |
| ATOM | 940 | N | GLU | A | 1011 | −14.836 | −1.306 | 29.382 | 1.00 | 39.80 | N |
| ATOM | 941 | CA | GLU | A | 1011 | −14.230 | −0.965 | 28.092 | 1.00 | 41.13 | C |
| ATOM | 942 | CB | GLU | A | 1011 | −13.008 | −1.847 | 27.835 | 1.00 | 42.83 | C |
| ATOM | 943 | CG | GLU | A | 1011 | −11.964 | −1.780 | 28.932 | 1.00 | 50.94 | C |
| ATOM | 944 | CD | GLU | A | 1011 | −10.624 | −2.334 | 28.491 | 1.00 | 58.98 | C |
| ATOM | 945 | OE1 | GLU | A | 1011 | −10.592 | −3.091 | 27.495 | 1.00 | 61.81 | O |
| ATOM | 946 | OE2 | GLU | A | 1011 | −9.603 | −2.002 | 29.135 | 1.00 | 48.46 | O |
| ATOM | 947 | C | GLU | A | 1011 | −15.190 | −1.057 | 26.907 | 1.00 | 49.02 | C |
| ATOM | 948 | O | GLU | A | 1011 | −14.972 | −0.417 | 25.888 | 1.00 | 46.25 | O |
| ATOM | 949 | N | GLY | A | 1012 | −16.243 | −1.872 | 27.024 | 1.00 | 51.32 | N |
| ATOM | 950 | CA | GLY | A | 1012 | −17.183 | −1.987 | 25.939 | 1.00 | 53.58 | C |
| ATOM | 951 | C | GLY | A | 1012 | −16.964 | −3.167 | 25.057 | 1.00 | 55.24 | C |
| ATOM | 952 | O | GLY | A | 1012 | −15.833 | −3.524 | 24.832 | 1.00 | 61.16 | O |
| ATOM | 953 | N | LEU | A | 1013 | −18.056 | −3.797 | 24.613 | 1.00 | 54.41 | N |
| ATOM | 954 | CA | LEU | A | 1013 | −18.073 | −5.011 | 23.771 | 1.00 | 62.61 | C |
| ATOM | 955 | CB | LEU | A | 1013 | −18.919 | −6.119 | 24.403 | 1.00 | 64.74 | C |
| ATOM | 956 | CG | LEU | A | 1013 | −19.015 | −7.388 | 23.554 | 1.00 | 55.03 | C |
| ATOM | 957 | CD1 | LEU | A | 1013 | −17.636 | −7.991 | 23.341 | 1.00 | 53.70 | C |
| ATOM | 958 | CD2 | LEU | A | 1013 | −19.953 | −8.402 | 24.184 | 1.00 | 59.63 | C |
| ATOM | 959 | C | LEU | A | 1013 | −18.549 | −4.749 | 22.333 | 1.00 | 65.57 | C |
| ATOM | 960 | O | LEU | A | 1013 | −19.702 | −4.415 | 22.126 | 1.00 | 73.33 | O |
| ATOM | 961 | N | ARG | A | 1014 | −17.663 | −4.876 | 21.346 | 1.00 | 65.31 | N |
| ATOM | 962 | CA | ARG | A | 1014 | −18.045 | −4.685 | 19.958 | 1.00 | 66.30 | C |
| ATOM | 963 | CB | ARG | A | 1014 | −17.263 | −3.531 | 19.324 | 1.00 | 63.32 | C |
| ATOM | 964 | CG | ARG | A | 1014 | −17.778 | −2.151 | 19.691 | 1.00 | 66.00 | C |
| ATOM | 965 | CD | ARG | A | 1014 | −17.337 | −1.101 | 18.676 | 1.00 | 68.21 | C |
| ATOM | 966 | NE | ARG | A | 1014 | −18.107 | 0.136 | 18.806 | 1.00 | 77.68 | N |
| ATOM | 967 | CZ | ARG | A | 1014 | −19.302 | 0.343 | 18.254 | 1.00 | 71.03 | C |
| ATOM | 968 | NH1 | ARG | A | 1014 | −19.921 | 1.500 | 18.428 | 1.00 | 62.85 | N |
| ATOM | 969 | NH2 | ARG | A | 1014 | −19.882 | −0.605 | 17.527 | 1.00 | 73.07 | N |
| ATOM | 970 | C | ARG | A | 1014 | −17.817 | −5.984 | 19.151 | 1.00 | 69.30 | C |
| ATOM | 971 | O | ARG | A | 1014 | −16.707 | −6.463 | 18.985 | 1.00 | 67.53 | O |
| ATOM | 972 | N | LEU | A | 1015 | −18.923 | −6.555 | 18.656 | 1.00 | 74.40 | N |
| ATOM | 973 | CA | LEU | A | 1015 | −18.931 | −7.909 | 18.103 | 1.00 | 79.08 | C |
| ATOM | 974 | CB | LEU | A | 1015 | −20.327 | −8.529 | 18.229 | 1.00 | 81.96 | C |
| ATOM | 975 | CG | LEU | A | 1015 | −20.828 | −8.765 | 19.659 | 1.00 | 76.89 | C |
| ATOM | 976 | CD1 | LEU | A | 1015 | −22.303 | −9.138 | 19.680 | 1.00 | 70.25 | C |
| ATOM | 977 | CD2 | LEU | A | 1015 | −19.995 | −9.835 | 20.349 | 1.00 | 77.80 | C |
| ATOM | 978 | C | LEU | A | 1015 | −18.455 | −7.957 | 16.649 | 1.00 | 86.75 | C |
| ATOM | 979 | O | LEU | A | 1015 | −18.196 | −9.036 | 16.101 | 1.00 | 94.78 | O |
| ATOM | 980 | N | LYS | A | 1016 | −18.324 | −6.765 | 16.057 | 1.00 | 84.04 | N |
| ATOM | 981 | CA | LYS | A | 1016 | −17.882 | −6.591 | 14.668 | 1.00 | 88.26 | C |
| ATOM | 982 | CB | LYS | A | 1016 | −18.853 | −5.674 | 13.911 | 1.00 | 91.53 | C |
| ATOM | 983 | CG | LYS | A | 1016 | −18.606 | −5.579 | 12.408 | 1.00 | 85.55 | C |
| ATOM | 984 | CD | LYS | A | 1016 | −19.695 | −4.784 | 11.706 | 1.00 | 75.35 | C |
| ATOM | 985 | CE | LYS | A | 1016 | −19.374 | −4.604 | 10.231 | 1.00 | 79.52 | C |
| ATOM | 986 | NZ | LYS | A | 1016 | −19.081 | −5.901 | 9.556 | 1.00 | 84.52 | N |
| ATOM | 987 | C | LYS | A | 1016 | −16.456 | −6.029 | 14.617 | 1.00 | 77.73 | C |
| ATOM | 988 | O | LYS | A | 1016 | −16.040 | −5.331 | 15.521 | 1.00 | 88.03 | O |
| ATOM | 989 | N | ILE | A | 1017 | −15.730 | −6.280 | 13.527 | 1.00 | 73.35 | N |
| ATOM | 990 | CA | ILE | A | 1017 | −14.389 | −5.736 | 13.383 | 1.00 | 73.63 | C |
| ATOM | 991 | CB | ILE | A | 1017 | −13.641 | −6.363 | 12.192 | 1.00 | 72.98 | C |
| ATOM | 992 | CG1 | ILE | A | 1017 | −13.291 | −7.823 | 12.484 | 1.00 | 75.55 | C |
| ATOM | 993 | CD1 | ILE | A | 1017 | −12.530 | −8.504 | 11.368 | 1.00 | 82.54 | C |
| ATOM | 994 | CG2 | ILE | A | 1017 | −12.379 | −5.583 | 11.893 | 1.00 | 74.93 | C |
| ATOM | 995 | C | ILE | A | 1017 | −14.465 | −4.234 | 13.195 | 1.00 | 71.10 | C |
| ATOM | 996 | O | ILE | A | 1017 | −15.213 | −3.737 | 12.374 | 1.00 | 70.90 | O |
| ATOM | 997 | N | TYR | A | 1018 | −13.700 | −3.522 | 14.018 | 1.00 | 69.84 | N |
| ATOM | 998 | CA | TYR | A | 1018 | −13.602 | −2.087 | 13.927 | 1.00 | 71.96 | C |
| ATOM | 999 | CB | TYR | A | 1018 | −14.454 | −1.405 | 15.002 | 1.00 | 72.18 | C |
| ATOM | 1000 | CG | TYR | A | 1018 | −13.981 | −1.632 | 16.418 | 1.00 | 72.48 | C |
| ATOM | 1001 | CD2 | TYR | A | 1018 | −13.200 | −0.686 | 17.072 | 1.00 | 76.57 | C |
| ATOM | 1002 | CE2 | TYR | A | 1018 | −12.770 | −0.888 | 18.368 | 1.00 | 74.68 | C |
| ATOM | 1003 | CZ | TYR | A | 1018 | −13.126 | −2.045 | 19.027 | 1.00 | 75.93 | C |
| ATOM | 1004 | OH | TYR | A | 1018 | −12.702 | −2.255 | 20.319 | 1.00 | 80.33 | O |
| ATOM | 1005 | CE1 | TYR | A | 1018 | −13.904 | −2.994 | 18.401 | 1.00 | 71.15 | C |
| ATOM | 1006 | CD1 | TYR | A | 1018 | −14.332 | −2.782 | 17.109 | 1.00 | 69.81 | C |
| ATOM | 1007 | C | TYR | A | 1018 | −12.140 | −1.686 | 14.042 | 1.00 | 75.03 | C |
| ATOM | 1008 | O | TYR | A | 1018 | −11.337 | −2.429 | 14.563 | 1.00 | 73.47 | O |
| ATOM | 1009 | N | LYS | A | 1019 | −11.807 | −0.506 | 13.547 | 1.00 | 73.56 | N |
| ATOM | 1010 | CA | LYS | A | 1019 | −10.474 | 0.044 | 13.696 | 1.00 | 68.66 | C |
| ATOM | 1011 | CB | LYS | A | 1019 | −10.115 | 0.937 | 12.506 | 1.00 | 71.11 | C |
| ATOM | 1012 | CG | LYS | A | 1019 | −9.949 | 0.228 | 11.176 | 1.00 | 70.75 | C |
| ATOM | 1013 | CD | LYS | A | 1019 | −9.615 | 1.242 | 10.088 | 1.00 | 77.61 | C |
| ATOM | 1014 | CE | LYS | A | 1019 | −9.504 | 0.596 | 8.717 | 1.00 | 96.50 | C |
| ATOM | 1015 | NZ | LYS | A | 1019 | −9.241 | 1.603 | 7.647 | 1.00 | 101.73 | N |
| ATOM | 1016 | C | LYS | A | 1019 | −10.353 | 0.867 | 14.985 | 1.00 | 65.35 | C |
| ATOM | 1017 | O | LYS | A | 1019 | −11.089 | 1.825 | 15.174 | 1.00 | 66.32 | O |
| ATOM | 1018 | N | ASP | A | 1020 | −9.348 | 0.567 | 15.813 | 1.00 | 62.66 | N |
| ATOM | 1019 | CA | ASP | A | 1020 | −9.112 | 1.352 | 17.010 | 1.00 | 62.72 | C |
| ATOM | 1020 | CB | ASP | A | 1020 | −8.205 | 0.608 | 18.006 | 1.00 | 65.27 | C |
| ATOM | 1021 | CG | ASP | A | 1020 | −6.766 | 0.488 | 17.529 | 1.00 | 68.11 | C |
| ATOM | 1022 | OD1 | ASP | A | 1020 | −6.502 | 0.720 | 16.330 | 1.00 | 66.96 | O |
| ATOM | 1023 | OD2 | ASP | A | 1020 | −5.896 | 0.150 | 18.363 | 1.00 | 70.27 | O |
| ATOM | 1024 | C | ASP | A | 1020 | −8.576 | 2.758 | 16.695 | 1.00 | 59.05 | C |
| ATOM | 1025 | O | ASP | A | 1020 | −8.575 | 3.182 | 15.550 | 1.00 | 54.61 | O |
| ATOM | 1026 | N | THR | A | 1021 | −8.198 | 3.490 | 17.755 | 1.00 | 58.41 | N |
| ATOM | 1027 | CA | THR | A | 1021 | −7.826 | 4.909 | 17.681 | 1.00 | 59.45 | C |
| ATOM | 1028 | CB | THR | A | 1021 | −7.532 | 5.499 | 19.076 | 1.00 | 62.60 | C |
| ATOM | 1029 | OG1 | THR | A | 1021 | −6.379 | 4.860 | 19.637 | 1.00 | 83.56 | O |
| ATOM | 1030 | CG2 | THR | A | 1021 | −8.726 | 5.299 | 20.001 | 1.00 | 50.61 | C |
| ATOM | 1031 | C | THR | A | 1021 | −6.655 | 5.196 | 16.730 | 1.00 | 62.90 | C |
| ATOM | 1032 | O | THR | A | 1021 | −6.642 | 6.175 | 16.002 | 1.00 | 59.00 | O |
| ATOM | 1033 | N | GLU | A | 1022 | −5.692 | 4.281 | 16.710 | 1.00 | 63.97 | N |
| ATOM | 1034 | CA | GLU | A | 1022 | −4.535 | 4.409 | 15.861 | 1.00 | 61.80 | C |
| ATOM | 1035 | CB | GLU | A | 1022 | −3.263 | 4.041 | 16.628 | 1.00 | 64.11 | C |
| ATOM | 1036 | CG | GLU | A | 1022 | −3.350 | 2.791 | 17.472 | 1.00 | 68.32 | C |
| ATOM | 1037 | CD | GLU | A | 1022 | −2.210 | 2.708 | 18.466 | 1.00 | 78.22 | C |
| ATOM | 1038 | OE1 | GLU | A | 1022 | −1.639 | 3.768 | 18.812 | 1.00 | 61.65 | O |
| ATOM | 1039 | OE2 | GLU | A | 1022 | −1.879 | 1.585 | 18.899 | 1.00 | 97.60 | O |
| ATOM | 1040 | C | GLU | A | 1022 | −4.653 | 3.603 | 14.571 | 1.00 | 64.72 | C |
| ATOM | 1041 | O | GLU | A | 1022 | −3.777 | 3.658 | 13.716 | 1.00 | 64.36 | O |
| ATOM | 1042 | N | GLY | A | 1023 | −5.811 | 2.974 | 14.378 | 1.00 | 59.06 | N |
| ATOM | 1043 | CA | GLY | A | 1023 | −6.151 | 2.435 | 13.100 | 1.00 | 59.35 | C |
| ATOM | 1044 | C | GLY | A | 1023 | −5.913 | 0.987 | 12.923 | 1.00 | 66.34 | C |
| ATOM | 1045 | O | GLY | A | 1023 | −6.131 | 0.493 | 11.830 | 1.00 | 73.61 | O |
| ATOM | 1046 | N | TYR | A | 1024 | −5.522 | 0.291 | 13.986 | 1.00 | 68.47 | N |
| ATOM | 1047 | CA | TYR | A | 1024 | −5.345 | −1.161 | 13.982 | 1.00 | 70.11 | C |
| ATOM | 1048 | CB | TYR | A | 1024 | −4.430 | −1.591 | 15.128 | 1.00 | 77.38 | C |
| ATOM | 1049 | CG | TYR | A | 1024 | −3.041 | −0.998 | 15.167 | 1.00 | 71.96 | C |
| ATOM | 1050 | CD2 | TYR | A | 1024 | −2.337 | −0.961 | 16.363 | 1.00 | 67.43 | C |
| ATOM | 1051 | CE2 | TYR | A | 1024 | −1.072 | −0.438 | 16.431 | 1.00 | 78.47 | C |
| ATOM | 1052 | CZ | TYR | A | 1024 | −0.480 | 0.059 | 15.293 | 1.00 | 82.49 | C |
| ATOM | 1053 | OH | TYR | A | 1024 | 0.793 | 0.576 | 15.376 | 1.00 | 76.11 | O |
| ATOM | 1054 | CE1 | TYR | A | 1024 | −1.153 | 0.033 | 14.087 | 1.00 | 76.05 | C |
| ATOM | 1055 | CD1 | TYR | A | 1024 | −2.428 | −0.498 | 14.027 | 1.00 | 68.89 | C |
| ATOM | 1056 | C | TYR | A | 1024 | −6.671 | −1.878 | 14.192 | 1.00 | 65.66 | C |
| ATOM | 1057 | O | TYR | A | 1024 | −7.490 | −1.411 | 14.942 | 1.00 | 67.20 | O |
| ATOM | 1058 | N | TYR | A | 1025 | −6.889 | −2.995 | 13.502 | 1.00 | 73.99 | N |
| ATOM | 1059 | CA | TYR | A | 1025 | −8.144 | −3.742 | 13.614 | 1.00 | 75.93 | C |
| ATOM | 1060 | CB | TYR | A | 1025 | −8.283 | −4.739 | 12.458 | 1.00 | 78.51 | C |
| ATOM | 1061 | CG | TYR | A | 1025 | −8.316 | −4.087 | 11.092 | 1.00 | 86.51 | C |
| ATOM | 1062 | CD2 | TYR | A | 1025 | −7.184 | −4.057 | 10.283 | 1.00 | 88.79 | C |
| ATOM | 1063 | CE2 | TYR | A | 1025 | −7.213 | −3.459 | 9.035 | 1.00 | 90.39 | C |
| ATOM | 1064 | CZ | TYR | A | 1025 | −8.382 | −2.884 | 8.583 | 1.00 | 97.90 | C |
| ATOM | 1065 | OH | TYR | A | 1025 | −8.416 | −2.289 | 7.342 | 1.00 | 105.91 | O |
| ATOM | 1066 | CE1 | TYR | A | 1025 | −9.518 | −2.904 | 9.366 | 1.00 | 94.71 | C |
| ATOM | 1067 | CD1 | TYR | A | 1025 | −9.481 | −3.501 | 10.612 | 1.00 | 87.27 | C |
| ATOM | 1068 | C | TYR | A | 1025 | −8.276 | −4.467 | 14.961 | 1.00 | 73.28 | C |
| ATOM | 1069 | O | TYR | A | 1025 | −7.361 | −5.125 | 15.426 | 1.00 | 70.09 | O |
| ATOM | 1070 | N | THR | A | 1026 | −9.427 | −4.250 | 15.595 | 1.00 | 76.03 | N |
| ATOM | 1071 | CA | THR | A | 1026 | −9.699 | −4.653 | 16.966 | 1.00 | 72.27 | C |
| ATOM | 1072 | CB | THR | A | 1026 | −9.481 | −3.472 | 17.939 | 1.00 | 73.11 | C |
| ATOM | 1073 | OG1 | THR | A | 1026 | −8.088 | −3.134 | 17.985 | 1.00 | 77.88 | O |
| ATOM | 1074 | CG2 | THR | A | 1026 | −9.963 | −3.815 | 19.343 | 1.00 | 76.20 | C |
| ATOM | 1075 | C | THR | A | 1026 | −11.157 | −5.130 | 17.046 | 1.00 | 80.79 | C |
| ATOM | 1076 | O | THR | A | 1026 | −12.029 | −4.537 | 16.424 | 1.00 | 81.39 | O |
| ATOM | 1077 | N | ILE | A | 1027 | −11.399 | −6.240 | 17.768 | 1.00 | 81.51 | N |
| ATOM | 1078 | CA | ILE | A | 1027 | −12.743 | −6.773 | 17.970 | 1.00 | 75.55 | C |
| ATOM | 1079 | CB | ILE | A | 1027 | −13.062 | −7.899 | 16.945 | 1.00 | 86.92 | C |
| ATOM | 1080 | CG1 | ILE | A | 1027 | −14.538 | −8.300 | 17.005 | 1.00 | 81.30 | C |
| ATOM | 1081 | CD1 | ILE | A | 1027 | −14.986 | −9.156 | 15.835 | 1.00 | 80.82 | C |
| ATOM | 1082 | CG2 | ILE | A | 1027 | −12.138 | −9.103 | 17.134 | 1.00 | 79.75 | C |
| ATOM | 1083 | C | ILE | A | 1027 | −12.931 | −7.241 | 19.390 | 1.00 | 68.90 | C |
| ATOM | 1084 | O | ILE | A | 1027 | −12.078 | −7.899 | 19.928 | 1.00 | 75.26 | O |
| ATOM | 1085 | N | GLY | A | 1028 | −14.085 | −7.002 | 19.959 | 1.00 | 65.93 | N |
| ATOM | 1086 | CA | GLY | A | 1028 | −14.404 | −7.584 | 21.237 | 1.00 | 62.61 | C |
| ATOM | 1087 | C | GLY | A | 1028 | −14.197 | −6.641 | 22.344 | 1.00 | 63.11 | C |
| ATOM | 1088 | O | GLY | A | 1028 | −14.590 | −5.490 | 22.221 | 1.00 | 62.96 | O |
| ATOM | 1089 | N | ILE | A | 1029 | −13.542 | −7.082 | 23.412 | 1.00 | 64.35 | N |
| ATOM | 1090 | CA | ILE | A | 1029 | −13.234 | −6.201 | 24.518 | 1.00 | 59.05 | C |
| ATOM | 1091 | CB | ILE | A | 1029 | −13.520 | −6.890 | 25.868 | 1.00 | 47.17 | C |
| ATOM | 1092 | CG1 | ILE | A | 1029 | −14.936 | −7.472 | 25.874 | 1.00 | 54.83 | C |
| ATOM | 1093 | CD1 | ILE | A | 1029 | −15.291 | −8.201 | 27.158 | 1.00 | 61.17 | C |
| ATOM | 1094 | CG2 | ILE | A | 1029 | −13.338 | −5.918 | 27.016 | 1.00 | 43.75 | C |
| ATOM | 1095 | C | ILE | A | 1029 | −11.775 | −5.781 | 24.445 | 1.00 | 61.60 | C |
| ATOM | 1096 | O | ILE | A | 1029 | −10.952 | −6.238 | 25.206 | 1.00 | 69.13 | O |
| ATOM | 1097 | N | GLY | A | 1030 | −11.447 | −4.950 | 23.472 | 1.00 | 62.47 | N |
| ATOM | 1098 | CA | GLY | A | 1030 | −10.109 | −4.487 | 23.285 | 1.00 | 64.05 | C |
| ATOM | 1099 | C | GLY | A | 1030 | −9.112 | −5.519 | 22.839 | 1.00 | 66.63 | C |
| ATOM | 1100 | O | GLY | A | 1030 | −8.005 | −5.526 | 23.349 | 1.00 | 76.20 | O |
| ATOM | 1101 | N | HIS | A | 1031 | −9.458 | −6.375 | 21.884 | 1.00 | 58.95 | N |
| ATOM | 1102 | CA | HIS | A | 1031 | −8.521 | −7.364 | 21.375 | 1.00 | 63.28 | C |
| ATOM | 1103 | CB | HIS | A | 1031 | −9.215 | −8.724 | 21.297 | 1.00 | 59.46 | C |
| ATOM | 1104 | CG | HIS | A | 1031 | −8.332 | −9.826 | 20.803 | 1.00 | 64.61 | C |
| ATOM | 1105 | ND1 | HIS | A | 1031 | −7.401 | −10.446 | 21.604 | 1.00 | 66.15 | N |
| ATOM | 1106 | CE1 | HIS | A | 1031 | −6.770 | −11.373 | 20.900 | 1.00 | 70.98 | C |
| ATOM | 1107 | NE2 | HIS | A | 1031 | −7.261 | −11.372 | 19.676 | 1.00 | 72.66 | N |
| ATOM | 1108 | CD2 | HIS | A | 1031 | −8.242 | −10.412 | 19.586 | 1.00 | 69.46 | C |
| ATOM | 1109 | C | HIS | A | 1031 | −8.029 | −6.955 | 20.004 | 1.00 | 71.25 | C |
| ATOM | 1110 | O | HIS | A | 1031 | −8.843 | −6.747 | 19.130 | 1.00 | 70.42 | O |
| ATOM | 1111 | N | LEU | A | 1032 | −6.692 | −6.822 | 19.841 | 1.00 | 75.55 | N |
| ATOM | 1112 | CA | LEU | A | 1032 | −6.071 | −6.423 | 18.576 | 1.00 | 73.48 | C |
| ATOM | 1113 | CB | LEU | A | 1032 | −4.806 | −5.601 | 18.850 | 1.00 | 77.05 | C |
| ATOM | 1114 | CG | LEU | A | 1032 | −3.771 | −5.473 | 17.726 | 1.00 | 77.31 | C |
| ATOM | 1115 | CD1 | LEU | A | 1032 | −4.344 | −4.766 | 16.500 | 1.00 | 79.99 | C |
| ATOM | 1116 | CD2 | LEU | A | 1032 | −2.516 | −4.766 | 18.224 | 1.00 | 65.04 | C |
| ATOM | 1117 | C | LEU | A | 1032 | −5.736 | −7.642 | 17.738 | 1.00 | 74.08 | C |
| ATOM | 1118 | O | LEU | A | 1032 | −5.175 | −8.609 | 18.214 | 1.00 | 75.30 | O |
| ATOM | 1119 | N | LEU | A | 1033 | −6.159 | −7.624 | 16.488 | 1.00 | 74.15 | N |
| ATOM | 1120 | CA | LEU | A | 1033 | −5.955 | −8.776 | 15.646 | 1.00 | 86.29 | C |
| ATOM | 1121 | CB | LEU | A | 1033 | −7.078 | −8.877 | 14.611 | 1.00 | 85.60 | C |
| ATOM | 1122 | CG | LEU | A | 1033 | −8.468 | −9.100 | 15.209 | 1.00 | 79.89 | C |
| ATOM | 1123 | CD1 | LEU | A | 1033 | −9.556 | −9.002 | 14.152 | 1.00 | 85.06 | C |
| ATOM | 1124 | CD2 | LEU | A | 1033 | −8.518 | −10.449 | 15.903 | 1.00 | 81.77 | C |
| ATOM | 1125 | C | LEU | A | 1033 | −4.579 | −8.727 | 14.955 | 1.00 | 92.56 | C |
| ATOM | 1126 | O | LEU | A | 1033 | −3.736 | −9.595 | 15.155 | 1.00 | 83.53 | O |
| ATOM | 1127 | N | THR | A | 1034 | −4.336 | −7.658 | 14.199 | 1.00 | 91.92 | N |
| ATOM | 1128 | CA | THR | A | 1034 | −3.044 | −7.467 | 13.579 | 1.00 | 95.63 | C |
| ATOM | 1129 | CB | THR | A | 1034 | −2.873 | −8.384 | 12.352 | 1.00 | 100.46 | C |
| ATOM | 1130 | OG1 | THR | A | 1034 | −1.523 | −8.313 | 11.876 | 1.00 | 102.11 | O |
| ATOM | 1131 | CG2 | THR | A | 1034 | −3.832 | −7.972 | 11.239 | 1.00 | 98.60 | C |
| ATOM | 1132 | C | THR | A | 1034 | −2.918 | −6.011 | 13.132 | 1.00 | 95.94 | C |
| ATOM | 1133 | O | THR | A | 1034 | −3.843 | −5.228 | 13.299 | 1.00 | 87.28 | O |
| ATOM | 1134 | N | LYS | A | 1035 | −1.737 | −5.651 | 12.620 | 1.00 | 96.41 | N |
| ATOM | 1135 | CA | LYS | A | 1035 | −1.441 | −4.278 | 12.272 | 1.00 | 88.52 | C |
| ATOM | 1136 | CB | LYS | A | 1035 | −0.193 | −3.800 | 13.014 | 1.00 | 74.50 | C |
| ATOM | 1137 | CG | LYS | A | 1035 | −0.297 | −3.912 | 14.524 | 1.00 | 75.34 | C |
| ATOM | 1138 | CD | LYS | A | 1035 | 0.939 | −3.350 | 15.207 | 1.00 | 72.03 | C |
| ATOM | 1139 | CE | LYS | A | 1035 | 0.819 | −3.436 | 16.720 | 1.00 | 70.16 | C |
| ATOM | 1140 | NZ | LYS | A | 1035 | 1.967 | −2.780 | 17.405 | 1.00 | 75.22 | N |
| ATOM | 1141 | C | LYS | A | 1035 | −1.260 | −4.107 | 10.757 | 1.00 | 95.34 | C |
| ATOM | 1142 | O | LYS | A | 1035 | −0.589 | −3.195 | 10.280 | 1.00 | 101.32 | O |
| ATOM | 1143 | N | SER | A | 1036 | −1.927 | −4.966 | 9.994 | 1.00 | 96.44 | N |
| ATOM | 1144 | CA | SER | A | 1036 | −1.925 | −4.831 | 8.550 | 1.00 | 98.54 | C |
| ATOM | 1145 | CB | SER | A | 1036 | −2.248 | −6.174 | 7.892 | 1.00 | 101.52 | C |
| ATOM | 1146 | OG | SER | A | 1036 | −2.417 | −6.036 | 6.492 | 1.00 | 104.06 | O |
| ATOM | 1147 | C | SER | A | 1036 | −2.971 | −3.788 | 8.152 | 1.00 | 105.10 | C |
| ATOM | 1148 | O | SER | A | 1036 | −3.952 | −3.604 | 8.848 | 1.00 | 111.78 | O |
| ATOM | 1149 | N | PRO | A | 1037 | −2.809 | −3.024 | 7.088 | 1.00 | 105.40 | N |
| ATOM | 1150 | CA | PRO | A | 1037 | −3.765 | −1.950 | 6.803 | 1.00 | 107.96 | C |
| ATOM | 1151 | CB | PRO | A | 1037 | −2.982 | −1.016 | 5.876 | 1.00 | 106.93 | C |
| ATOM | 1152 | CG | PRO | A | 1037 | −1.545 | −1.389 | 6.072 | 1.00 | 99.64 | C |
| ATOM | 1153 | CD | PRO | A | 1037 | −1.553 | −2.858 | 6.323 | 1.00 | 104.57 | C |
| ATOM | 1154 | C | PRO | A | 1037 | −4.976 | −2.564 | 6.072 | 1.00 | 108.80 | C |
| ATOM | 1155 | O | PRO | A | 1037 | −5.959 | −1.852 | 5.852 | 1.00 | 104.27 | O |
| ATOM | 1156 | N | SER | A | 1038 | −4.853 | −3.832 | 5.626 | 1.00 | 104.53 | N |
| ATOM | 1157 | CA | SER | A | 1038 | −5.893 | −4.494 | 4.828 | 1.00 | 108.57 | C |
| ATOM | 1158 | CB | SER | A | 1038 | −5.259 | −5.373 | 3.745 | 1.00 | 108.89 | C |
| ATOM | 1159 | OG | SER | A | 1038 | −6.245 | −6.024 | 2.962 | 1.00 | 113.57 | O |
| ATOM | 1160 | C | SER | A | 1038 | −6.843 | −5.331 | 5.707 | 1.00 | 113.16 | C |
| ATOM | 1161 | O | SER | A | 1038 | −6.460 | −6.360 | 6.250 | 1.00 | 111.43 | O |
| ATOM | 1162 | N | LEU | A | 1039 | −8.108 | −4.866 | 5.811 | 1.00 | 112.72 | N |
| ATOM | 1163 | CA | LEU | A | 1039 | −9.166 | −5.555 | 6.569 | 1.00 | 108.34 | C |
| ATOM | 1164 | CB | LEU | A | 1039 | −10.483 | −4.778 | 6.441 | 1.00 | 105.61 | C |
| ATOM | 1165 | CG | LEU | A | 1039 | −11.793 | −5.465 | 6.842 | 1.00 | 106.47 | C |
| ATOM | 1166 | CD1 | LEU | A | 1039 | −11.806 | −5.792 | 8.325 | 1.00 | 99.38 | C |
| ATOM | 1167 | CD2 | LEU | A | 1039 | −13.000 | −4.613 | 6.464 | 1.00 | 107.77 | C |
| ATOM | 1168 | C | LEU | A | 1039 | −9.363 | −6.997 | 6.102 | 1.00 | 110.84 | C |
| ATOM | 1169 | O | LEU | A | 1039 | −9.751 | −7.862 | 6.874 | 1.00 | 110.79 | O |
| ATOM | 1170 | N | SER | A | 1040 | −9.040 | −7.244 | 4.825 | 1.00 | 110.40 | N |
| ATOM | 1171 | CA | SER | A | 1040 | −9.069 | −8.587 | 4.263 | 1.00 | 109.46 | C |
| ATOM | 1172 | CB | SER | A | 1040 | −8.647 | −8.551 | 2.792 | 1.00 | 104.53 | C |
| ATOM | 1173 | OG | SER | A | 1040 | −9.502 | −7.713 | 2.033 | 1.00 | 103.76 | O |
| ATOM | 1174 | C | SER | A | 1040 | −8.148 | −9.527 | 5.058 | 1.00 | 114.85 | C |
| ATOM | 1175 | O | SER | A | 1040 | −8.558 | −10.590 | 5.530 | 1.00 | 122.32 | O |
| ATOM | 1176 | N | VAL | A | 1041 | −6.918 | −9.055 | 5.280 | 1.00 | 110.51 | N |
| ATOM | 1177 | CA | VAL | A | 1041 | −5.962 | −9.751 | 6.113 | 1.00 | 108.15 | C |
| ATOM | 1178 | CB | VAL | A | 1041 | −4.713 | −8.937 | 6.182 | 1.00 | 110.29 | C |
| ATOM | 1179 | CG1 | VAL | A | 1041 | −3.671 | −9.642 | 7.052 | 1.00 | 108.83 | C |
| ATOM | 1180 | CG2 | VAL | A | 1041 | −4.268 | −8.653 | 4.757 | 1.00 | 108.64 | C |
| ATOM | 1181 | C | VAL | A | 1041 | −6.487 | −9.912 | 7.541 | 1.00 | 109.89 | C |
| ATOM | 1182 | O | VAL | A | 1041 | −6.307 | −10.956 | 8.146 | 1.00 | 110.19 | O |
| ATOM | 1183 | N | ALA | A | 1042 | −7.161 | −8.876 | 8.066 | 1.00 | 112.89 | N |
| ATOM | 1184 | CA | ALA | A | 1042 | −7.694 | −8.930 | 9.441 | 1.00 | 117.14 | C |
| ATOM | 1185 | CB | ALA | A | 1042 | −8.277 | −7.580 | 9.834 | 1.00 | 110.58 | C |
| ATOM | 1186 | C | ALA | A | 1042 | −8.731 | −10.047 | 9.641 | 1.00 | 110.68 | C |
| ATOM | 1187 | O | ALA | A | 1042 | −8.699 | −10.773 | 10.625 | 1.00 | 107.93 | O |
| ATOM | 1188 | N | LYS | A | 1043 | −9.653 | −10.187 | 8.692 | 1.00 | 109.73 | N |
| ATOM | 1189 | CA | LYS | A | 1043 | −10.639 | −11.263 | 8.764 | 1.00 | 111.13 | C |
| ATOM | 1190 | CB | LYS | A | 1043 | −11.572 | −11.221 | 7.550 | 1.00 | 110.46 | C |
| ATOM | 1191 | CG | LYS | A | 1043 | −12.358 | −9.919 | 7.453 | 1.00 | 110.30 | C |
| ATOM | 1192 | CD | LYS | A | 1043 | −13.281 | −9.875 | 6.246 | 1.00 | 107.80 | C |
| ATOM | 1193 | CE | LYS | A | 1043 | −14.096 | −8.586 | 6.246 | 1.00 | 95.65 | C |
| ATOM | 1194 | NZ | LYS | A | 1043 | −15.001 | −8.476 | 5.070 | 1.00 | 84.35 | N |
| ATOM | 1195 | C | LYS | A | 1043 | −9.984 | −12.652 | 8.929 | 1.00 | 114.80 | C |
| ATOM | 1196 | O | LYS | A | 1043 | −10.398 | −13.483 | 9.736 | 1.00 | 114.93 | O |
| ATOM | 1197 | N | SER | A | 1044 | −8.903 | −12.858 | 8.188 | 1.00 | 120.39 | N |
| ATOM | 1198 | CA | SER | A | 1044 | −8.137 | −14.088 | 8.293 | 1.00 | 119.20 | C |
| ATOM | 1199 | CB | SER | A | 1044 | −7.044 | −14.135 | 7.221 | 1.00 | 110.65 | C |
| ATOM | 1200 | OG | SER | A | 1044 | −7.600 | −14.096 | 5.919 | 1.00 | 107.72 | O |
| ATOM | 1201 | C | SER | A | 1044 | −7.502 | −14.266 | 9.675 | 1.00 | 117.22 | C |
| ATOM | 1202 | O | SER | A | 1044 | −7.522 | −15.354 | 10.245 | 1.00 | 119.44 | O |
| ATOM | 1203 | N | GLU | A | 1045 | −6.886 | −13.192 | 10.181 | 1.00 | 117.08 | N |
| ATOM | 1204 | CA | GLU | A | 1045 | −6.186 | −13.245 | 11.470 | 1.00 | 117.62 | C |
| ATOM | 1205 | CB | GLU | A | 1045 | −5.403 | −11.958 | 11.757 | 1.00 | 118.46 | C |
| ATOM | 1206 | CG | GLU | A | 1045 | −4.541 | −11.441 | 10.599 | 1.00 | 118.79 | C |
| ATOM | 1207 | CD | GLU | A | 1045 | −3.443 | −12.398 | 10.133 | 1.00 | 120.12 | C |
| ATOM | 1208 | OE1 | GLU | A | 1045 | −3.263 | −13.483 | 10.730 | 1.00 | 122.48 | O |
| ATOM | 1209 | OE2 | GLU | A | 1045 | −2.746 | −12.049 | 9.153 | 1.00 | 114.87 | O |
| ATOM | 1210 | C | GLU | A | 1045 | −7.116 | −13.600 | 12.629 | 1.00 | 115.86 | C |
| ATOM | 1211 | O | GLU | A | 1045 | −6.728 | −14.283 | 13.575 | 1.00 | 112.94 | O |
| ATOM | 1212 | N | LEU | A | 1046 | −8.370 | −13.183 | 12.498 | 1.00 | 111.29 | N |
| ATOM | 1213 | CA | LEU | A | 1046 | −9.387 | −13.536 | 13.465 | 1.00 | 110.38 | C |
| ATOM | 1214 | CB | LEU | A | 1046 | −10.639 | −12.686 | 13.247 | 1.00 | 110.34 | C |
| ATOM | 1215 | CG | LEU | A | 1046 | −11.813 | −12.881 | 14.206 | 1.00 | 101.53 | C |
| ATOM | 1216 | CD1 | LEU | A | 1046 | −11.373 | −12.726 | 15.654 | 1.00 | 92.96 | C |
| ATOM | 1217 | CD2 | LEU | A | 1046 | −12.915 | −11.892 | 13.869 | 1.00 | 100.03 | C |
| ATOM | 1218 | C | LEU | A | 1046 | −9.722 | −15.030 | 13.379 | 1.00 | 111.84 | C |
| ATOM | 1219 | O | LEU | A | 1046 | −9.938 | −15.707 | 14.380 | 1.00 | 106.63 | O |
| ATOM | 1220 | N | ASP | A | 1047 | −9.767 | −15.527 | 12.140 | 1.00 | 116.67 | N |
| ATOM | 1221 | CA | ASP | A | 1047 | −10.065 | −16.934 | 11.887 | 1.00 | 116.00 | C |
| ATOM | 1222 | CB | ASP | A | 1047 | −10.135 | −17.204 | 10.383 | 1.00 | 123.30 | C |
| ATOM | 1223 | CG | ASP | A | 1047 | −11.196 | −16.372 | 9.695 | 1.00 | 129.31 | C |
| ATOM | 1224 | OD1 | ASP | A | 1047 | −12.135 | −15.927 | 10.388 | 1.00 | 126.87 | O |
| ATOM | 1225 | OD2 | ASP | A | 1047 | −11.095 | −16.163 | 8.467 | 1.00 | 130.31 | O |
| ATOM | 1226 | C | ASP | A | 1047 | −9.060 | −17.883 | 12.533 | 1.00 | 110.88 | C |
| ATOM | 1227 | O | ASP | A | 1047 | −9.448 | −18.848 | 13.163 | 1.00 | 111.90 | O |
| ATOM | 1228 | N | LYS | A | 1048 | −7.770 | −17.582 | 12.438 | 1.00 | 111.88 | N |
| ATOM | 1229 | CA | LYS | A | 1048 | −6.775 | −18.417 | 13.120 | 1.00 | 111.15 | C |
| ATOM | 1230 | CB | LYS | A | 1048 | −5.352 | −18.010 | 12.810 | 1.00 | 115.45 | C |
| ATOM | 1231 | CG | LYS | A | 1048 | −4.442 | −19.020 | 13.389 | 1.00 | 111.32 | C |
| ATOM | 1232 | CD | LYS | A | 1048 | −3.034 | −18.764 | 13.085 | 1.00 | 104.91 | C |
| ATOM | 1233 | CE | LYS | A | 1048 | −2.266 | −19.837 | 13.781 | 1.00 | 106.12 | C |
| ATOM | 1234 | NZ | LYS | A | 1048 | −1.027 | −19.278 | 14.386 | 1.00 | 117.10 | N |
| ATOM | 1235 | C | LYS | A | 1048 | −7.010 | −18.433 | 14.646 | 1.00 | 112.86 | C |
| ATOM | 1236 | O | LYS | A | 1048 | −6.857 | −19.460 | 15.307 | 1.00 | 107.35 | O |
| ATOM | 1237 | N | ALA | A | 1049 | −7.326 | −17.235 | 15.184 | 1.00 | 113.67 | N |
| ATOM | 1238 | CA | ALA | A | 1049 | −7.542 | −17.037 | 16.619 | 1.00 | 100.83 | C |
| ATOM | 1239 | CB | ALA | A | 1049 | −7.727 | −15.557 | 16.925 | 1.00 | 92.23 | C |
| ATOM | 1240 | C | ALA | A | 1049 | −8.723 | −17.841 | 17.162 | 1.00 | 100.82 | C |
| ATOM | 1241 | O | ALA | A | 1049 | −8.627 | −18.500 | 18.196 | 1.00 | 88.66 | O |
| ATOM | 1242 | N | ILE | A | 1050 | −9.864 | −17.679 | 16.477 | 1.00 | 111.04 | N |
| ATOM | 1243 | CA | ILE | A | 1050 | −11.122 | −18.309 | 16.855 | 1.00 | 103.06 | C |
| ATOM | 1244 | CB | ILE | A | 1050 | −12.306 | −17.526 | 16.210 | 1.00 | 94.28 | C |
| ATOM | 1245 | CG1 | ILE | A | 1050 | −12.609 | −16.269 | 17.021 | 1.00 | 90.34 | C |
| ATOM | 1246 | CD1 | ILE | A | 1050 | −12.914 | −16.542 | 18.467 | 1.00 | 83.38 | C |
| ATOM | 1247 | CG2 | ILE | A | 1050 | −13.566 | −18.361 | 16.091 | 1.00 | 100.57 | C |
| ATOM | 1248 | C | ILE | A | 1050 | −11.183 | −19.788 | 16.478 | 1.00 | 107.29 | C |
| ATOM | 1249 | O | ILE | A | 1050 | −11.926 | −20.566 | 17.076 | 1.00 | 106.04 | O |
| ATOM | 1250 | N | GLY | A | 1051 | −10.454 | −20.142 | 15.420 | 1.00 | 114.24 | N |
| ATOM | 1251 | CA | GLY | A | 1051 | −10.471 | −21.485 | 14.856 | 1.00 | 115.33 | C |
| ATOM | 1252 | C | GLY | A | 1051 | −11.326 | −21.592 | 13.569 | 1.00 | 117.27 | C |
| ATOM | 1253 | O | GLY | A | 1051 | −11.149 | −22.538 | 12.803 | 1.00 | 107.96 | O |
| ATOM | 1254 | N | ARG | A | 1052 | −12.317 | −20.691 | 13.395 | 1.00 | 119.25 | N |
| ATOM | 1255 | CA | ARG | A | 1052 | −13.267 | −20.791 | 12.284 | 1.00 | 122.88 | C |
| ATOM | 1256 | CB | ARG | A | 1052 | −14.666 | −21.099 | 12.837 | 1.00 | 123.98 | C |
| ATOM | 1257 | CG | ARG | A | 1052 | −15.740 | −21.345 | 11.785 | 1.00 | 131.04 | C |
| ATOM | 1258 | CD | ARG | A | 1052 | −17.115 | −21.535 | 12.413 | 1.00 | 132.70 | C |
| ATOM | 1259 | NE | ARG | A | 1052 | −17.553 | −20.359 | 13.166 | 1.00 | 131.76 | N |
| ATOM | 1260 | CZ | ARG | A | 1052 | −18.171 | −19.310 | 12.625 | 1.00 | 131.14 | C |
| ATOM | 1261 | NH1 | ARG | A | 1052 | −18.539 | −18.287 | 13.387 | 1.00 | 113.42 | N |
| ATOM | 1262 | NH2 | ARG | A | 1052 | −18.420 | −19.282 | 11.321 | 1.00 | 131.92 | N |
| ATOM | 1263 | C | ARG | A | 1052 | −13.303 | −19.493 | 11.445 | 1.00 | 127.06 | C |
| ATOM | 1264 | O | ARG | A | 1052 | −13.046 | −18.405 | 11.949 | 1.00 | 124.85 | O |
| ATOM | 1265 | N | ASN | A | 1053 | −13.728 | −19.614 | 10.166 | 1.00 | 127.07 | N |
| ATOM | 1266 | CA | ASN | A | 1053 | −13.973 | −18.437 | 9.316 | 1.00 | 121.06 | C |
| ATOM | 1267 | CB | ASN | A | 1053 | −14.327 | −18.860 | 7.893 | 1.00 | 125.39 | C |
| ATOM | 1268 | CG | ASN | A | 1053 | −14.347 | −17.691 | 6.931 | 1.00 | 134.13 | C |
| ATOM | 1269 | OD1 | ASN | A | 1053 | −13.678 | −16.680 | 7.154 | 1.00 | 134.90 | O |
| ATOM | 1270 | ND2 | ASN | A | 1053 | −15.114 | −17.820 | 5.854 | 1.00 | 137.98 | N |
| ATOM | 1271 | C | ASN | A | 1053 | −15.077 | −17.522 | 9.889 | 1.00 | 124.60 | C |
| ATOM | 1272 | O | ASN | A | 1053 | −16.099 | −17.999 | 10.385 | 1.00 | 122.11 | O |
| ATOM | 1273 | N | SER | A | 1054 | −14.759 | −16.215 | 9.956 | 1.00 | 128.06 | N |
| ATOM | 1274 | CA | SER | A | 1054 | −15.555 | −15.261 | 10.736 | 1.00 | 129.56 | C |
| ATOM | 1275 | CB | SER | A | 1054 | −14.651 | −14.438 | 11.661 | 1.00 | 124.33 | C |
| ATOM | 1276 | OG | SER | A | 1054 | −13.662 | −13.734 | 10.926 | 1.00 | 118.04 | O |
| ATOM | 1277 | C | SER | A | 1054 | −16.430 | −14.319 | 9.878 | 1.00 | 124.12 | C |
| ATOM | 1278 | O | SER | A | 1054 | −17.501 | −13.886 | 10.317 | 1.00 | 118.04 | O |
| ATOM | 1279 | N | ASN | A | 1055 | −15.893 | −13.920 | 8.709 | 1.00 | 123.06 | N |
| ATOM | 1280 | CA | ASN | A | 1055 | −16.520 | −12.896 | 7.854 | 1.00 | 127.74 | C |
| ATOM | 1281 | CB | ASN | A | 1055 | −17.898 | −13.343 | 7.335 | 1.00 | 134.09 | C |
| ATOM | 1282 | CG | ASN | A | 1055 | −17.882 | −14.739 | 6.729 | 1.00 | 135.97 | C |
| ATOM | 1283 | OD1 | ASN | A | 1055 | −17.247 | −14.976 | 5.701 | 1.00 | 139.23 | O |
| ATOM | 1284 | ND2 | ASN | A | 1055 | −18.599 | −15.667 | 7.361 | 1.00 | 123.39 | N |
| ATOM | 1285 | C | ASN | A | 1055 | −16.634 | −11.508 | 8.565 | 1.00 | 114.77 | C |
| ATOM | 1286 | O | ASN | A | 1055 | −17.322 | −10.608 | 8.074 | 1.00 | 107.53 | O |
| ATOM | 1287 | N | GLY | A | 1056 | −15.906 | −11.327 | 9.692 | 1.00 | 110.70 | N |
| ATOM | 1288 | CA | GLY | A | 1056 | −15.850 | −10.039 | 10.357 | 1.00 | 107.13 | C |
| ATOM | 1289 | C | GLY | A | 1056 | −16.591 | −9.965 | 11.701 | 1.00 | 103.85 | C |
| ATOM | 1290 | O | GLY | A | 1056 | −16.515 | −8.934 | 12.383 | 1.00 | 96.52 | O |
| ATOM | 1291 | N | VAL | A | 1057 | −17.450 | −10.971 | 11.970 | 1.00 | 110.95 | N |
| ATOM | 1292 | CA | VAL | A | 1057 | −18.389 | −10.935 | 13.114 | 1.00 | 105.94 | C |
| ATOM | 1293 | CB | VAL | A | 1057 | −19.843 | −10.665 | 12.670 | 1.00 | 102.07 | C |
| ATOM | 1294 | CG1 | VAL | A | 1057 | −20.821 | −10.967 | 13.804 | 1.00 | 87.18 | C |
| ATOM | 1295 | CG2 | VAL | A | 1057 | −19.990 | −9.228 | 12.204 | 1.00 | 95.57 | C |
| ATOM | 1296 | C | VAL | A | 1057 | −18.327 | −12.234 | 13.940 | 1.00 | 103.37 | C |
| ATOM | 1297 | O | VAL | A | 1057 | −18.466 | −13.329 | 13.394 | 1.00 | 108.03 | O |
| ATOM | 1298 | N | ILE | A | 1058 | −18.011 | −12.100 | 15.238 | 1.00 | 96.30 | N |
| ATOM | 1299 | CA | ILE | A | 1058 | −17.853 | −13.261 | 16.134 | 1.00 | 94.95 | C |
| ATOM | 1300 | CB | ILE | A | 1058 | −16.506 | −13.252 | 16.895 | 1.00 | 95.28 | C |
| ATOM | 1301 | CG1 | ILE | A | 1058 | −16.330 | −11.948 | 17.677 | 1.00 | 92.93 | C |
| ATOM | 1302 | CD1 | ILE | A | 1058 | −15.098 | −11.927 | 18.560 | 1.00 | 86.49 | C |
| ATOM | 1303 | CG2 | ILE | A | 1058 | −15.353 | −13.471 | 15.933 | 1.00 | 93.92 | C |
| ATOM | 1304 | C | ILE | A | 1058 | −19.002 | −13.367 | 17.129 | 1.00 | 90.26 | C |
| ATOM | 1305 | O | ILE | A | 1058 | −19.490 | −12.369 | 17.645 | 1.00 | 79.23 | O |
| ATOM | 1306 | N | THR | A | 1059 | −19.389 | −14.609 | 17.442 | 1.00 | 91.10 | N |
| ATOM | 1307 | CA | THR | A | 1059 | −20.420 | −14.841 | 18.451 | 1.00 | 99.03 | C |
| ATOM | 1308 | CB | THR | A | 1059 | −20.810 | −16.330 | 18.526 | 1.00 | 92.30 | C |
| ATOM | 1309 | OG1 | THR | A | 1059 | −19.691 | −17.099 | 18.982 | 1.00 | 84.72 | O |
| ATOM | 1310 | CG2 | THR | A | 1059 | −21.249 | −16.838 | 17.159 | 1.00 | 96.11 | C |
| ATOM | 1311 | C | THR | A | 1059 | −19.963 | −14.361 | 19.858 | 1.00 | 95.67 | C |
| ATOM | 1312 | O | THR | A | 1059 | −18.786 | −14.390 | 20.167 | 1.00 | 84.72 | O |
| ATOM | 1313 | N | LYS | A | 1060 | −20.926 | −13.988 | 20.717 | 1.00 | 97.06 | N |
| ATOM | 1314 | CA | LYS | A | 1060 | −20.643 | −13.595 | 22.108 | 1.00 | 84.20 | C |
| ATOM | 1315 | CB | LYS | A | 1060 | −21.939 | −13.302 | 22.867 | 1.00 | 69.95 | C |
| ATOM | 1316 | CG | LYS | A | 1060 | −21.722 | −12.536 | 24.158 | 1.00 | 61.71 | C |
| ATOM | 1317 | CD | LYS | A | 1060 | −23.039 | −12.172 | 24.823 | 1.00 | 74.53 | C |
| ATOM | 1318 | CE | LYS | A | 1060 | −23.841 | −11.183 | 23.987 | 1.00 | 72.90 | C |
| ATOM | 1319 | NZ | LYS | A | 1060 | −25.131 | −10.819 | 24.648 | 1.00 | 65.59 | N |
| ATOM | 1320 | C | LYS | A | 1060 | −19.764 | −14.594 | 22.888 | 1.00 | 79.33 | C |
| ATOM | 1321 | O | LYS | A | 1060 | −18.816 | −14.224 | 23.552 | 1.00 | 71.30 | O |
| ATOM | 1322 | N | ASP | A | 1061 | −20.050 | −15.880 | 22.695 | 1.00 | 91.58 | N |
| ATOM | 1323 | CA | ASP | A | 1061 | −19.291 | −16.981 | 23.306 | 1.00 | 94.72 | C |
| ATOM | 1324 | CB | ASP | A | 1061 | −20.004 | −18.323 | 23.092 | 1.00 | 98.69 | C |
| ATOM | 1325 | CG | ASP | A | 1061 | −19.670 | −19.350 | 24.171 | 1.00 | 94.82 | C |
| ATOM | 1326 | OD1 | ASP | A | 1061 | −18.519 | −19.363 | 24.660 | 1.00 | 89.35 | O |
| ATOM | 1327 | OD2 | ASP | A | 1061 | −20.568 | −20.145 | 24.528 | 1.00 | 83.50 | O |
| ATOM | 1328 | C | ASP | A | 1061 | −17.837 | −17.039 | 22.765 | 1.00 | 82.97 | C |
| ATOM | 1329 | O | ASP | A | 1061 | −16.900 | −17.422 | 23.453 | 1.00 | 77.65 | O |
| ATOM | 1330 | N | GLU | A | 1062 | −17.667 | −16.609 | 21.512 | 1.00 | 84.00 | N |
| ATOM | 1331 | CA | GLU | A | 1062 | −16.340 | −16.510 | 20.902 | 1.00 | 91.88 | C |
| ATOM | 1332 | CB | GLU | A | 1062 | −16.433 | −16.429 | 19.372 | 1.00 | 96.32 | C |
| ATOM | 1333 | CG | GLU | A | 1062 | −16.564 | −17.799 | 18.699 | 1.00 | 98.43 | C |
| ATOM | 1334 | CD | GLU | A | 1062 | −16.784 | −17.708 | 17.200 | 1.00 | 100.05 | C |
| ATOM | 1335 | OE1 | GLU | A | 1062 | −17.041 | −16.589 | 16.706 | 1.00 | 100.42 | O |
| ATOM | 1336 | OE2 | GLU | A | 1062 | −16.701 | −18.757 | 16.521 | 1.00 | 92.44 | O |
| ATOM | 1337 | C | GLU | A | 1062 | −15.516 | −15.337 | 21.506 | 1.00 | 82.68 | C |
| ATOM | 1338 | O | GLU | A | 1062 | −14.327 | −15.458 | 21.753 | 1.00 | 73.52 | O |
| ATOM | 1339 | N | ALA | A | 1063 | −16.179 | −14.201 | 21.770 | 1.00 | 81.24 | N |
| ATOM | 1340 | CA | ALA | A | 1063 | −15.526 | −13.034 | 22.355 | 1.00 | 66.34 | C |
| ATOM | 1341 | CB | ALA | A | 1063 | −16.494 | −11.868 | 22.435 | 1.00 | 57.64 | C |
| ATOM | 1342 | C | ALA | A | 1063 | −14.976 | −13.363 | 23.732 | 1.00 | 68.86 | C |
| ATOM | 1343 | O | ALA | A | 1063 | −13.874 | −12.997 | 24.091 | 1.00 | 70.75 | O |
| ATOM | 1344 | N | GLU | A | 1064 | −15.739 | −14.162 | 24.453 | 1.00 | 69.59 | N |
| ATOM | 1345 | CA | GLU | A | 1064 | −15.336 | −14.694 | 25.729 | 1.00 | 69.24 | C |
| ATOM | 1346 | CB | GLU | A | 1064 | −16.412 | −15.661 | 26.239 | 1.00 | 71.73 | C |
| ATOM | 1347 | CG | GLU | A | 1064 | −16.051 | −16.443 | 27.493 | 1.00 | 70.48 | C |
| ATOM | 1348 | CD | GLU | A | 1064 | −16.318 | −15.679 | 28.771 | 1.00 | 73.86 | C |
| ATOM | 1349 | OE1 | GLU | A | 1064 | −16.983 | −14.623 | 28.723 | 1.00 | 78.00 | O |
| ATOM | 1350 | OE2 | GLU | A | 1064 | −15.861 | −16.150 | 29.834 | 1.00 | 73.15 | O |
| ATOM | 1351 | C | GLU | A | 1064 | −13.977 | −15.418 | 25.618 | 1.00 | 73.35 | C |
| ATOM | 1352 | O | GLU | A | 1064 | −13.079 | −15.140 | 26.377 | 1.00 | 68.15 | O |
| ATOM | 1353 | N | LYS | A | 1065 | −13.814 | −16.321 | 24.647 | 1.00 | 72.09 | N |
| ATOM | 1354 | CA | LYS | A | 1065 | −12.530 | −17.008 | 24.441 | 1.00 | 67.53 | C |
| ATOM | 1355 | CB | LYS | A | 1065 | −12.648 | −18.088 | 23.362 | 1.00 | 70.56 | C |
| ATOM | 1356 | CG | LYS | A | 1065 | −13.532 | −19.255 | 23.786 | 1.00 | 83.14 | C |
| ATOM | 1357 | CD | LYS | A | 1065 | −13.379 | −20.460 | 22.872 | 1.00 | 86.97 | C |
| ATOM | 1358 | CE | LYS | A | 1065 | −14.114 | −21.668 | 23.438 | 1.00 | 77.79 | C |
| ATOM | 1359 | NZ | LYS | A | 1065 | −13.986 | −22.862 | 22.558 | 1.00 | 88.60 | N |
| ATOM | 1360 | C | LYS | A | 1065 | −11.355 | −16.064 | 24.162 | 1.00 | 67.17 | C |
| ATOM | 1361 | O | LYS | A | 1065 | −10.291 | −16.197 | 24.747 | 1.00 | 63.18 | O |
| ATOM | 1362 | N | LEU | A | 1066 | −11.589 | −15.060 | 23.313 | 1.00 | 67.68 | N |
| ATOM | 1363 | CA | LEU | A | 1066 | −10.607 | −14.014 | 23.051 | 1.00 | 62.00 | C |
| ATOM | 1364 | CB | LEU | A | 1066 | −11.117 | −13.033 | 21.994 | 1.00 | 59.36 | C |
| ATOM | 1365 | CG | LEU | A | 1066 | −11.072 | −13.521 | 20.544 | 1.00 | 62.31 | C |
| ATOM | 1366 | CD1 | LEU | A | 1066 | −11.572 | −12.443 | 19.600 | 1.00 | 63.74 | C |
| ATOM | 1367 | CD2 | LEU | A | 1066 | −9.663 | −13.956 | 20.162 | 1.00 | 58.11 | C |
| ATOM | 1368 | C | LEU | A | 1066 | −10.225 | −13.279 | 24.328 | 1.00 | 61.55 | C |
| ATOM | 1369 | O | LEU | A | 1066 | −9.062 | −13.099 | 24.626 | 1.00 | 56.88 | O |
| ATOM | 1370 | N | PHE | A | 1067 | −11.243 | −12.863 | 25.078 | 1.00 | 61.07 | N |
| ATOM | 1371 | CA | PHE | A | 1067 | −11.070 | −12.191 | 26.365 | 1.00 | 56.02 | C |
| ATOM | 1372 | CB | PHE | A | 1067 | −12.416 | −11.728 | 26.919 | 1.00 | 57.11 | C |
| ATOM | 1373 | CG | PHE | A | 1067 | −12.313 | −11.003 | 28.227 | 1.00 | 52.81 | C |
| ATOM | 1374 | CD1 | PHE | A | 1067 | −12.027 | −9.653 | 28.261 | 1.00 | 48.02 | C |
| ATOM | 1375 | CE1 | PHE | A | 1067 | −11.937 | −8.990 | 29.460 | 1.00 | 48.08 | C |
| ATOM | 1376 | CZ | PHE | A | 1067 | −12.131 | −9.672 | 30.649 | 1.00 | 46.23 | C |
| ATOM | 1377 | CE2 | PHE | A | 1067 | −12.414 | −11.012 | 30.631 | 1.00 | 48.02 | C |
| ATOM | 1378 | CD2 | PHE | A | 1067 | −12.506 | −11.673 | 29.425 | 1.00 | 54.19 | C |
| ATOM | 1379 | C | PHE | A | 1067 | −10.324 | −13.046 | 27.410 | 1.00 | 55.70 | C |
| ATOM | 1380 | O | PHE | A | 1067 | −9.445 | −12.573 | 28.089 | 1.00 | 56.06 | O |
| ATOM | 1381 | N | ASN | A | 1068 | −10.696 | −14.310 | 27.546 | 1.00 | 57.07 | N |
| ATOM | 1382 | CA | ASN | A | 1068 | −10.018 | −15.227 | 28.444 | 1.00 | 56.35 | C |
| ATOM | 1383 | CB | ASN | A | 1068 | −10.711 | −16.587 | 28.460 | 1.00 | 59.50 | C |
| ATOM | 1384 | CG | ASN | A | 1068 | −12.072 | −16.531 | 29.125 | 1.00 | 71.69 | C |
| ATOM | 1385 | OD1 | ASN | A | 1068 | −12.325 | −15.677 | 29.979 | 1.00 | 68.82 | O |
| ATOM | 1386 | ND2 | ASN | A | 1068 | −12.957 | −17.439 | 28.739 | 1.00 | 81.73 | N |
| ATOM | 1387 | C | ASN | A | 1068 | −8.550 | −15.354 | 28.105 | 1.00 | 62.98 | C |
| ATOM | 1388 | O | ASN | A | 1068 | −7.705 | −15.300 | 28.974 | 1.00 | 63.39 | O |
| ATOM | 1389 | N | GLN | A | 1069 | −8.250 | −15.429 | 26.813 | 1.00 | 66.16 | N |
| ATOM | 1390 | CA | GLN | A | 1069 | −6.873 | −15.410 | 26.347 | 1.00 | 62.61 | C |
| ATOM | 1391 | CB | GLN | A | 1069 | −6.816 | −15.544 | 24.824 | 1.00 | 60.30 | C |
| ATOM | 1392 | CG | GLN | A | 1069 | −7.249 | −16.909 | 24.311 | 1.00 | 64.02 | C |
| ATOM | 1393 | CD | GLN | A | 1069 | −7.281 | −16.978 | 22.798 | 1.00 | 67.59 | C |
| ATOM | 1394 | OE1 | GLN | A | 1069 | −6.928 | −16.016 | 22.117 | 1.00 | 70.65 | O |
| ATOM | 1395 | NE2 | GLN | A | 1069 | −7.707 | −18.119 | 22.263 | 1.00 | 68.94 | N |
| ATOM | 1396 | C | GLN | A | 1069 | −6.086 | −14.160 | 26.804 | 1.00 | 58.40 | C |
| ATOM | 1397 | O | GLN | A | 1069 | −4.988 | −14.245 | 27.321 | 1.00 | 55.12 | O |
| ATOM | 1398 | N | ASP | A | 1070 | −6.687 | −12.992 | 26.641 | 1.00 | 59.42 | N |
| ATOM | 1399 | CA | ASP | A | 1070 | −6.086 | −11.740 | 27.057 | 1.00 | 53.26 | C |
| ATOM | 1400 | CB | ASP | A | 1070 | −6.971 | −10.565 | 26.637 | 1.00 | 51.89 | C |
| ATOM | 1401 | CG | ASP | A | 1070 | −7.130 | −10.468 | 25.131 | 1.00 | 64.76 | C |
| ATOM | 1402 | OD1 | ASP | A | 1070 | −6.167 | −10.808 | 24.410 | 1.00 | 68.52 | O |
| ATOM | 1403 | OD2 | ASP | A | 1070 | −8.216 | −10.054 | 24.669 | 1.00 | 63.08 | O |
| ATOM | 1404 | C | ASP | A | 1070 | −5.807 | −11.674 | 28.565 | 1.00 | 57.24 | C |
| ATOM | 1405 | O | ASP | A | 1070 | −4.797 | −11.130 | 29.004 | 1.00 | 62.78 | O |
| ATOM | 1406 | N | VAL | A | 1071 | −6.732 | −12.212 | 29.356 | 1.00 | 58.57 | N |
| ATOM | 1407 | CA | VAL | A | 1071 | −6.598 | −12.260 | 30.802 | 1.00 | 58.99 | C |
| ATOM | 1408 | CB | VAL | A | 1071 | −7.878 | −12.804 | 31.475 | 1.00 | 56.86 | C |
| ATOM | 1409 | CG1 | VAL | A | 1071 | −7.644 | −13.074 | 32.957 | 1.00 | 52.65 | C |
| ATOM | 1410 | CG2 | VAL | A | 1071 | −9.016 | −11.820 | 31.290 | 1.00 | 54.48 | C |
| ATOM | 1411 | C | VAL | A | 1071 | −5.391 | −13.069 | 31.231 | 1.00 | 58.09 | C |
| ATOM | 1412 | O | VAL | A | 1071 | −4.678 | −12.681 | 32.127 | 1.00 | 51.09 | O |
| ATOM | 1413 | N | ASP | A | 1072 | −5.159 | −14.198 | 30.558 | 1.00 | 65.79 | N |
| ATOM | 1414 | CA | ASP | A | 1072 | −3.978 | −15.034 | 30.799 | 1.00 | 60.58 | C |
| ATOM | 1415 | CB | ASP | A | 1072 | −4.061 | −16.339 | 30.007 | 1.00 | 62.40 | C |
| ATOM | 1416 | CG | ASP | A | 1072 | −5.063 | −17.311 | 30.598 | 1.00 | 77.54 | C |
| ATOM | 1417 | OD2 | ASP | A | 1072 | −5.471 | −18.257 | 29.891 | 1.00 | 80.83 | O |
| ATOM | 1418 | OD1 | ASP | A | 1072 | −5.438 | −17.133 | 31.776 | 1.00 | 82.25 | O |
| ATOM | 1419 | C | ASP | A | 1072 | −2.705 | −14.300 | 30.457 | 1.00 | 60.47 | C |
| ATOM | 1420 | O | ASP | A | 1072 | −1.759 | −14.335 | 31.202 | 1.00 | 63.46 | O |
| ATOM | 1421 | N | ALA | A | 1073 | −2.716 | −13.584 | 29.343 | 1.00 | 60.66 | N |
| ATOM | 1422 | CA | ALA | A | 1073 | −1.610 | −12.716 | 28.986 | 1.00 | 54.14 | C |
| ATOM | 1423 | CB | ALA | A | 1073 | −1.865 | −12.078 | 27.644 | 1.00 | 52.55 | C |
| ATOM | 1424 | C | ALA | A | 1073 | −1.366 | −11.652 | 30.038 | 1.00 | 55.05 | C |
| ATOM | 1425 | O | ALA | A | 1073 | −0.254 | −11.415 | 30.443 | 1.00 | 58.28 | O |
| ATOM | 1426 | N | ALA | A | 1074 | −2.428 | −11.062 | 30.543 | 1.00 | 54.15 | N |
| ATOM | 1427 | CA | ALA | A | 1074 | −2.319 | −10.100 | 31.613 | 1.00 | 53.44 | C |
| ATOM | 1428 | CB | ALA | A | 1074 | −3.675 | −9.491 | 31.910 | 1.00 | 47.57 | C |
| ATOM | 1429 | C | ALA | A | 1074 | −1.731 | −10.709 | 32.874 | 1.00 | 53.36 | C |
| ATOM | 1430 | O | ALA | A | 1074 | −0.866 | −10.154 | 33.502 | 1.00 | 53.73 | O |
| ATOM | 1431 | N | VAL | A | 1075 | −2.173 | −11.886 | 33.236 | 1.00 | 53.59 | N |
| ATOM | 1432 | CA | VAL | A | 1075 | −1.606 | −12.566 | 34.381 | 1.00 | 52.41 | C |
| ATOM | 1433 | CB | VAL | A | 1075 | −2.384 | −13.865 | 34.706 | 1.00 | 47.10 | C |
| ATOM | 1434 | CG1 | VAL | A | 1075 | −1.667 | −14.687 | 35.768 | 1.00 | 46.82 | C |
| ATOM | 1435 | CG2 | VAL | A | 1075 | −3.785 | −13.524 | 35.166 | 1.00 | 45.45 | C |
| ATOM | 1436 | C | VAL | A | 1075 | −0.106 | −12.873 | 34.205 | 1.00 | 53.99 | C |
| ATOM | 1437 | O | VAL | A | 1075 | 0.670 | −12.710 | 35.124 | 1.00 | 55.39 | O |
| ATOM | 1438 | N | ARG | A | 1076 | 0.299 | −13.290 | 33.004 | 1.00 | 58.90 | N |
| ATOM | 1439 | CA | ARG | A | 1076 | 1.699 | −13.592 | 32.701 | 1.00 | 57.02 | C |
| ATOM | 1440 | CB | ARG | A | 1076 | 1.857 | −14.110 | 31.267 | 1.00 | 56.00 | C |
| ATOM | 1441 | CG | ARG | A | 1076 | 1.306 | −15.513 | 31.066 | 1.00 | 57.59 | C |
| ATOM | 1442 | CD | ARG | A | 1076 | 1.533 | −16.023 | 29.654 | 1.00 | 57.59 | C |
| ATOM | 1443 | NE | ARG | A | 1076 | 0.704 | −15.331 | 28.671 | 1.00 | 64.43 | N |
| ATOM | 1444 | CZ | ARG | A | 1076 | 0.620 | −15.678 | 27.389 | 1.00 | 76.73 | C |
| ATOM | 1445 | NH1 | ARG | A | 1076 | 1.313 | −16.714 | 26.933 | 1.00 | 94.42 | N |
| ATOM | 1446 | NH2 | ARG | A | 1076 | −0.156 | −14.992 | 26.561 | 1.00 | 70.82 | N |
| ATOM | 1447 | C | ARG | A | 1076 | 2.559 | −12.393 | 32.928 | 1.00 | 52.64 | C |
| ATOM | 1448 | O | ARG | A | 1076 | 3.593 | −12.473 | 33.559 | 1.00 | 55.31 | O |
| ATOM | 1449 | N | GLY | A | 1077 | 2.078 | −11.271 | 32.446 | 1.00 | 48.31 | N |
| ATOM | 1450 | CA | GLY | A | 1077 | 2.682 | −10.000 | 32.650 | 1.00 | 51.61 | C |
| ATOM | 1451 | C | GLY | A | 1077 | 2.743 | −9.611 | 34.079 | 1.00 | 52.46 | C |
| ATOM | 1452 | O | GLY | A | 1077 | 3.733 | −9.051 | 34.510 | 1.00 | 56.49 | O |
| ATOM | 1453 | N | ILE | A | 1078 | 1.683 | −9.889 | 34.831 | 1.00 | 50.33 | N |
| ATOM | 1454 | CA | ILE | A | 1078 | 1.639 | −9.553 | 36.229 | 1.00 | 52.25 | C |
| ATOM | 1455 | CB | ILE | A | 1078 | 0.217 | −9.703 | 36.808 | 1.00 | 53.76 | C |
| ATOM | 1456 | CG1 | ILE | A | 1078 | −0.707 | −8.649 | 36.191 | 1.00 | 48.85 | C |
| ATOM | 1457 | CD1 | ILE | A | 1078 | −2.136 | −8.702 | 36.691 | 1.00 | 48.42 | C |
| ATOM | 1458 | CG2 | ILE | A | 1078 | 0.233 | −9.552 | 38.323 | 1.00 | 50.92 | C |
| ATOM | 1459 | C | ILE | A | 1078 | 2.650 | −10.368 | 37.039 | 1.00 | 48.60 | C |
| ATOM | 1460 | O | ILE | A | 1078 | 3.378 | −9.853 | 37.863 | 1.00 | 46.91 | O |
| ATOM | 1461 | N | LEU | A | 1079 | 2.698 | −11.654 | 36.773 | 1.00 | 45.84 | N |
| ATOM | 1462 | CA | LEU | A | 1079 | 3.613 | −12.551 | 37.454 | 1.00 | 54.79 | C |
| ATOM | 1463 | CB | LEU | A | 1079 | 3.267 | −14.013 | 37.154 | 1.00 | 56.46 | C |
| ATOM | 1464 | CG | LEU | A | 1079 | 1.930 | −14.460 | 37.751 | 1.00 | 45.95 | C |
| ATOM | 1465 | CD1 | LEU | A | 1079 | 1.661 | −15.930 | 37.472 | 1.00 | 40.90 | C |
| ATOM | 1466 | CD2 | LEU | A | 1079 | 1.896 | −14.177 | 39.247 | 1.00 | 49.57 | C |
| ATOM | 1467 | C | LEU | A | 1079 | 5.078 | −12.261 | 37.170 | 1.00 | 61.79 | C |
| ATOM | 1468 | O | LEU | A | 1079 | 5.912 | −12.396 | 38.042 | 1.00 | 59.79 | O |
| ATOM | 1469 | N | ARG | A | 1080 | 5.407 | −11.891 | 35.933 | 1.00 | 65.81 | N |
| ATOM | 1470 | CA | ARG | A | 1080 | 6.763 | −11.471 | 35.617 | 1.00 | 53.79 | C |
| ATOM | 1471 | CB | ARG | A | 1080 | 6.991 | −11.459 | 34.106 | 1.00 | 51.31 | C |
| ATOM | 1472 | CG | ARG | A | 1080 | 6.799 | −12.847 | 33.504 | 1.00 | 64.77 | C |
| ATOM | 1473 | CD | ARG | A | 1080 | 6.994 | −12.877 | 32.003 | 1.00 | 73.12 | C |
| ATOM | 1474 | NE | ARG | A | 1080 | 8.370 | −12.568 | 31.620 | 1.00 | 81.76 | N |
| ATOM | 1475 | CZ | ARG | A | 1080 | 8.818 | −12.602 | 30.369 | 1.00 | 80.50 | C |
| ATOM | 1476 | NH1 | ARG | A | 1080 | 10.084 | −12.305 | 30.099 | 1.00 | 77.50 | N |
| ATOM | 1477 | NH2 | ARG | A | 1080 | 7.995 | −12.936 | 29.384 | 1.00 | 78.45 | N |
| ATOM | 1478 | C | ARG | A | 1080 | 7.177 | −10.152 | 36.290 | 1.00 | 55.76 | C |
| ATOM | 1479 | O | ARG | A | 1080 | 8.323 | −9.996 | 36.687 | 1.00 | 68.19 | O |
| ATOM | 1480 | N | ASN | A | 1081 | 6.234 | −9.237 | 36.493 | 1.00 | 52.29 | N |
| ATOM | 1481 | CA | ASN | A | 1081 | 6.501 | −7.980 | 37.208 | 1.00 | 52.29 | C |
| ATOM | 1482 | CB | ASN | A | 1081 | 5.369 | −6.969 | 37.004 | 1.00 | 53.72 | C |
| ATOM | 1483 | CG | ASN | A | 1081 | 5.794 | −5.545 | 37.338 | 1.00 | 52.84 | C |
| ATOM | 1484 | OD1 | ASN | A | 1081 | 6.318 | −5.277 | 38.420 | 1.00 | 52.77 | O |
| ATOM | 1485 | ND2 | ASN | A | 1081 | 5.583 | −4.630 | 36.400 | 1.00 | 52.45 | N |
| ATOM | 1486 | C | ASN | A | 1081 | 6.817 | −8.144 | 38.716 | 1.00 | 50.33 | C |
| ATOM | 1487 | O | ASN | A | 1081 | 6.000 | −8.556 | 39.503 | 1.00 | 54.07 | O |
| ATOM | 1488 | N | ALA | A | 1082 | 8.018 | −7.749 | 39.103 | 1.00 | 57.74 | N |
| ATOM | 1489 | CA | ALA | A | 1082 | 8.519 | −7.856 | 40.470 | 1.00 | 57.74 | C |
| ATOM | 1490 | CB | ALA | A | 1082 | 9.960 | −7.366 | 40.519 | 1.00 | 62.42 | C |
| ATOM | 1491 | C | ALA | A | 1082 | 7.669 | −7.117 | 41.513 | 1.00 | 49.91 | C |
| ATOM | 1492 | O | ALA | A | 1082 | 7.650 | −7.470 | 42.682 | 1.00 | 48.71 | O |
| ATOM | 1493 | N | LYS | A | 1083 | 7.066 | −6.018 | 41.091 | 1.00 | 50.90 | N |
| ATOM | 1494 | CA | LYS | A | 1083 | 6.303 | −5.157 | 41.983 | 1.00 | 54.74 | C |
| ATOM | 1495 | CB | LYS | A | 1083 | 6.283 | −3.718 | 41.461 | 1.00 | 50.27 | C |
| ATOM | 1496 | CG | LYS | A | 1083 | 7.658 | −3.073 | 41.342 | 1.00 | 50.10 | C |
| ATOM | 1497 | CD | LYS | A | 1083 | 7.552 | −1.656 | 40.799 | 1.00 | 50.24 | C |
| ATOM | 1498 | CE | LYS | A | 1083 | 8.881 | −0.926 | 40.870 | 1.00 | 57.97 | C |
| ATOM | 1499 | NZ | LYS | A | 1083 | 8.754 | 0.489 | 40.418 | 1.00 | 66.55 | N |
| ATOM | 1500 | C | LYS | A | 1083 | 4.878 | −5.672 | 42.195 | 1.00 | 57.56 | C |
| ATOM | 1501 | O | LYS | A | 1083 | 4.367 | −5.737 | 43.302 | 1.00 | 56.51 | O |
| ATOM | 1502 | N | LEU | A | 1084 | 4.254 | −6.040 | 41.091 | 1.00 | 52.93 | N |
| ATOM | 1503 | CA | LEU | A | 1084 | 2.893 | −6.498 | 41.074 | 1.00 | 47.71 | C |
| ATOM | 1504 | CB | LEU | A | 1084 | 2.327 | −6.435 | 39.650 | 1.00 | 45.24 | C |
| ATOM | 1505 | CG | LEU | A | 1084 | 2.386 | −5.059 | 38.976 | 1.00 | 45.18 | C |
| ATOM | 1506 | CD1 | LEU | A | 1084 | 1.734 | −5.087 | 37.600 | 1.00 | 41.18 | C |
| ATOM | 1507 | CD2 | LEU | A | 1084 | 1.744 | −3.990 | 39.851 | 1.00 | 43.90 | C |
| ATOM | 1508 | C | LEU | A | 1084 | 2.697 | −7.883 | 41.667 | 1.00 | 50.60 | C |
| ATOM | 1509 | O | LEU | A | 1084 | 1.652 | −8.177 | 42.206 | 1.00 | 51.26 | O |
| ATOM | 1510 | N | LYS | A | 1085 | 3.659 | −8.778 | 41.432 | 1.00 | 49.84 | N |
| ATOM | 1511 | CA | LYS | A | 1085 | 3.518 | −10.192 | 41.791 | 1.00 | 48.75 | C |
| ATOM | 1512 | CB | LYS | A | 1085 | 4.732 | −11.032 | 41.384 | 1.00 | 45.70 | C |
| ATOM | 1513 | CG | LYS | A | 1085 | 4.585 | −12.492 | 41.794 | 1.00 | 47.83 | C |
| ATOM | 1514 | CD | LYS | A | 1085 | 5.787 | −13.328 | 41.408 | 1.00 | 54.72 | C |
| ATOM | 1515 | CE | LYS | A | 1085 | 5.646 | −14.748 | 41.933 | 1.00 | 53.06 | C |
| ATOM | 1516 | NZ | LYS | A | 1085 | 5.580 | −14.786 | 43.420 | 1.00 | 61.28 | N |
| ATOM | 1517 | C | LYS | A | 1085 | 3.160 | −10.447 | 43.249 | 1.00 | 55.90 | C |
| ATOM | 1518 | O | LYS | A | 1085 | 2.231 | −11.201 | 43.504 | 1.00 | 56.10 | O |
| ATOM | 1519 | N | PRO | A | 1086 | 3.849 | −9.942 | 44.288 | 1.00 | 55.99 | N |
| ATOM | 1520 | CA | PRO | A | 1086 | 3.538 | −10.260 | 45.673 | 1.00 | 55.20 | C |
| ATOM | 1521 | CB | PRO | A | 1086 | 4.540 | −9.411 | 46.460 | 1.00 | 52.63 | C |
| ATOM | 1522 | CG | PRO | A | 1086 | 5.685 | −9.242 | 45.538 | 1.00 | 47.55 | C |
| ATOM | 1523 | CD | PRO | A | 1086 | 5.042 | −9.084 | 44.196 | 1.00 | 51.77 | C |
| ATOM | 1524 | C | PRO | A | 1086 | 2.106 | −9.842 | 46.008 | 1.00 | 52.74 | C |
| ATOM | 1525 | O | PRO | A | 1086 | 1.418 | −10.526 | 46.746 | 1.00 | 53.01 | O |
| ATOM | 1526 | N | VAL | A | 1087 | 1.707 | −8.698 | 45.479 | 1.00 | 53.51 | N |
| ATOM | 1527 | CA | VAL | A | 1087 | 0.399 | −8.147 | 45.706 | 1.00 | 53.66 | C |
| ATOM | 1528 | CB | VAL | A | 1087 | 0.278 | −6.716 | 45.139 | 1.00 | 51.02 | C |
| ATOM | 1529 | CG1 | VAL | A | 1087 | −1.015 | −6.062 | 45.618 | 1.00 | 46.03 | C |
| ATOM | 1530 | CG2 | VAL | A | 1087 | 1.484 | −5.881 | 45.538 | 1.00 | 59.26 | C |
| ATOM | 1531 | C | VAL | A | 1087 | −0.692 | −8.998 | 45.085 | 1.00 | 49.83 | C |
| ATOM | 1532 | O | VAL | A | 1087 | −1.677 | −9.319 | 45.710 | 1.00 | 48.38 | O |
| ATOM | 1533 | N | TYR | A | 1088 | −0.487 | −9.368 | 43.839 | 1.00 | 48.94 | N |
| ATOM | 1534 | CA | TYR | A | 1088 | −1.426 | −10.189 | 43.130 | 1.00 | 49.46 | C |
| ATOM | 1535 | CB | TYR | A | 1088 | −0.981 | −10.378 | 41.680 | 1.00 | 46.11 | C |
| ATOM | 1536 | CG | TYR | A | 1088 | −1.896 | −11.258 | 40.866 | 1.00 | 44.19 | C |
| ATOM | 1537 | CD1 | TYR | A | 1088 | −3.025 | −10.735 | 40.251 | 1.00 | 44.81 | C |
| ATOM | 1538 | CE1 | TYR | A | 1088 | −3.859 | −11.532 | 39.496 | 1.00 | 42.85 | C |
| ATOM | 1539 | CZ | TYR | A | 1088 | −3.569 | −12.869 | 39.348 | 1.00 | 44.23 | C |
| ATOM | 1540 | OH | TYR | A | 1088 | −4.403 | −13.661 | 38.595 | 1.00 | 47.10 | O |
| ATOM | 1541 | CE2 | TYR | A | 1088 | −2.452 | −13.415 | 39.947 | 1.00 | 45.57 | C |
| ATOM | 1542 | CD2 | TYR | A | 1088 | −1.623 | −12.610 | 40.698 | 1.00 | 40.75 | C |
| ATOM | 1543 | C | TYR | A | 1088 | −1.616 | −11.538 | 43.796 | 1.00 | 51.69 | C |
| ATOM | 1544 | O | TYR | A | 1088 | −2.722 | −12.029 | 43.883 | 1.00 | 57.19 | O |
| ATOM | 1545 | N | ASP | A | 1089 | −0.528 | −12.145 | 44.235 | 1.00 | 50.83 | N |
| ATOM | 1546 | CA | ASP | A | 1089 | −0.596 | −13.452 | 44.861 | 1.00 | 52.20 | C |
| ATOM | 1547 | CB | ASP | A | 1089 | 0.799 | −14.057 | 45.039 | 1.00 | 56.03 | C |
| ATOM | 1548 | CG | ASP | A | 1089 | 1.305 | −14.742 | 43.778 | 1.00 | 65.57 | C |
| ATOM | 1549 | OD1 | ASP | A | 1089 | 0.493 | −14.986 | 42.859 | 1.00 | 59.26 | O |
| ATOM | 1550 | OD2 | ASP | A | 1089 | 2.515 | −15.050 | 43.709 | 1.00 | 76.04 | O |
| ATOM | 1551 | C | ASP | A | 1089 | −1.346 | −13.426 | 46.187 | 1.00 | 47.28 | C |
| ATOM | 1552 | O | ASP | A | 1089 | −1.982 | −14.393 | 46.543 | 1.00 | 46.57 | O |
| ATOM | 1553 | N | SER | A | 1090 | −1.242 | −12.329 | 46.922 | 1.00 | 45.70 | N |
| ATOM | 1554 | CA | SER | A | 1090 | −1.943 | −12.193 | 48.190 | 1.00 | 47.54 | C |
| ATOM | 1555 | CB | SER | A | 1090 | −1.304 | −11.091 | 49.041 | 1.00 | 53.04 | C |
| ATOM | 1556 | OG | SER | A | 1090 | −1.677 | −9.803 | 48.579 | 1.00 | 53.10 | O |
| ATOM | 1557 | C | SER | A | 1090 | −3.454 | −11.924 | 48.046 | 1.00 | 52.01 | C |
| ATOM | 1558 | O | SER | A | 1090 | −4.227 | −12.169 | 48.963 | 1.00 | 52.95 | O |
| ATOM | 1559 | N | LEU | A | 1091 | −3.851 | −11.326 | 46.921 | 1.00 | 50.61 | N |
| ATOM | 1560 | CA | LEU | A | 1091 | −5.230 | −10.924 | 46.709 | 1.00 | 48.29 | C |
| ATOM | 1561 | CB | LEU | A | 1091 | −5.328 | −9.946 | 45.536 | 1.00 | 45.71 | C |
| ATOM | 1562 | CG | LEU | A | 1091 | −4.919 | −8.492 | 45.744 | 1.00 | 40.75 | C |
| ATOM | 1563 | CD1 | LEU | A | 1091 | −5.199 | −7.704 | 44.479 | 1.00 | 36.02 | C |
| ATOM | 1564 | CD2 | LEU | A | 1091 | −5.659 | −7.894 | 46.924 | 1.00 | 43.54 | C |
| ATOM | 1565 | C | LEU | A | 1091 | −6.114 | −12.118 | 46.411 | 1.00 | 51.68 | C |
| ATOM | 1566 | O | LEU | A | 1091 | −5.678 | −13.118 | 45.870 | 1.00 | 49.35 | O |
| ATOM | 1567 | N | ASP | A | 1092 | −7.386 | −11.976 | 46.761 | 1.00 | 53.56 | N |
| ATOM | 1568 | CA | ASP | A | 1092 | −8.399 | −12.964 | 46.452 | 1.00 | 52.79 | C |
| ATOM | 1569 | CB | ASP | A | 1092 | −9.554 | −12.815 | 47.434 | 1.00 | 46.89 | C |
| ATOM | 1570 | CG | ASP | A | 1092 | −9.976 | −11.371 | 47.605 | 1.00 | 52.31 | C |
| ATOM | 1571 | OD1 | ASP | A | 1092 | −9.229 | −10.617 | 48.268 | 1.00 | 51.97 | O |
| ATOM | 1572 | OD2 | ASP | A | 1092 | −11.042 | −10.989 | 47.074 | 1.00 | 52.49 | O |
| ATOM | 1573 | C | ASP | A | 1092 | −8.913 | −12.757 | 45.048 | 1.00 | 50.38 | C |
| ATOM | 1574 | O | ASP | A | 1092 | −8.516 | −11.825 | 44.392 | 1.00 | 52.48 | O |
| ATOM | 1575 | N | ALA | A | 1093 | −9.743 | −13.672 | 44.571 | 1.00 | 46.78 | N |
| ATOM | 1576 | CA | ALA | A | 1093 | −10.154 | −13.701 | 43.179 | 1.00 | 43.01 | C |
| ATOM | 1577 | CB | ALA | A | 1093 | −11.063 | −14.901 | 42.915 | 1.00 | 42.68 | C |
| ATOM | 1578 | C | ALA | A | 1093 | −10.827 | −12.412 | 42.698 | 1.00 | 40.00 | C |
| ATOM | 1579 | O | ALA | A | 1093 | −10.597 | −11.954 | 41.604 | 1.00 | 45.23 | O |
| ATOM | 1580 | N | VAL | A | 1094 | −11.703 | −11.835 | 43.494 | 1.00 | 43.98 | N |
| ATOM | 1581 | CA | VAL | A | 1094 | −12.424 | −10.666 | 43.046 | 1.00 | 41.38 | C |
| ATOM | 1582 | CB | VAL | A | 1094 | −13.667 | −10.371 | 43.867 | 1.00 | 45.80 | C |
| ATOM | 1583 | CG1 | VAL | A | 1094 | −14.490 | −9.286 | 43.180 | 1.00 | 47.65 | C |
| ATOM | 1584 | CG2 | VAL | A | 1094 | −14.497 | −11.638 | 44.026 | 1.00 | 45.84 | C |
| ATOM | 1585 | C | VAL | A | 1094 | −11.525 | −9.465 | 42.890 | 1.00 | 43.66 | C |
| ATOM | 1586 | O | VAL | A | 1094 | −11.598 | −8.771 | 41.902 | 1.00 | 46.51 | O |
| ATOM | 1587 | N | ARG | A | 1095 | −10.648 | −9.266 | 43.870 | 1.00 | 48.86 | N |
| ATOM | 1588 | CA | ARG | A | 1095 | −9.662 | −8.211 | 43.859 | 1.00 | 40.45 | C |
| ATOM | 1589 | CB | ARG | A | 1095 | −9.021 | −8.054 | 45.237 | 1.00 | 39.02 | C |
| ATOM | 1590 | CG | ARG | A | 1095 | −10.012 | −7.582 | 46.283 | 1.00 | 42.70 | C |
| ATOM | 1591 | CD | ARG | A | 1095 | −9.357 | −7.396 | 47.616 | 1.00 | 43.79 | C |
| ATOM | 1592 | NE | ARG | A | 1095 | −10.250 | −6.771 | 48.583 | 1.00 | 45.55 | N |
| ATOM | 1593 | CZ | ARG | A | 1095 | −11.045 | −7.440 | 49.411 | 1.00 | 47.33 | C |
| ATOM | 1594 | NH1 | ARG | A | 1095 | −11.073 | −8.767 | 49.386 | 1.00 | 48.81 | N |
| ATOM | 1595 | NH2 | ARG | A | 1095 | −11.813 | −6.777 | 50.266 | 1.00 | 45.04 | N |
| ATOM | 1596 | C | ARG | A | 1095 | −8.609 | −8.392 | 42.787 | 1.00 | 38.19 | C |
| ATOM | 1597 | O | ARG | A | 1095 | −8.184 | −7.454 | 42.167 | 1.00 | 41.93 | O |
| ATOM | 1598 | N | ARG | A | 1096 | −8.239 | −9.615 | 42.502 | 1.00 | 40.52 | N |
| ATOM | 1599 | CA | ARG | A | 1096 | −7.328 | −9.902 | 41.401 | 1.00 | 42.48 | C |
| ATOM | 1600 | CB | ARG | A | 1096 | −7.051 | −11.405 | 41.305 | 1.00 | 39.20 | C |
| ATOM | 1601 | CG | ARG | A | 1096 | −6.119 | −11.940 | 42.379 | 1.00 | 41.39 | C |
| ATOM | 1602 | CD | ARG | A | 1096 | −5.957 | −13.438 | 42.240 | 1.00 | 43.34 | C |
| ATOM | 1603 | NE | ARG | A | 1096 | −4.845 | −13.951 | 43.031 | 1.00 | 44.54 | N |
| ATOM | 1604 | CZ | ARG | A | 1096 | −4.313 | −15.157 | 42.860 | 1.00 | 52.63 | C |
| ATOM | 1605 | NH1 | ARG | A | 1096 | −4.794 | −15.965 | 41.924 | 1.00 | 56.57 | N |
| ATOM | 1606 | NH2 | ARG | A | 1096 | −3.299 | −15.553 | 43.619 | 1.00 | 55.22 | N |
| ATOM | 1607 | C | ARG | A | 1096 | −7.864 | −9.389 | 40.085 | 1.00 | 38.99 | C |
| ATOM | 1608 | O | ARG | A | 1096 | −7.153 | −8.825 | 39.292 | 1.00 | 41.38 | O |
| ATOM | 1609 | N | SER | A | 1097 | −9.166 | −9.556 | 39.911 | 1.00 | 40.83 | N |
| ATOM | 1610 | CA | SER | A | 1097 | −9.905 | −9.043 | 38.771 | 1.00 | 40.79 | C |
| ATOM | 1611 | CB | SER | A | 1097 | −11.370 | −9.484 | 38.827 | 1.00 | 40.93 | C |
| ATOM | 1612 | OG | SER | A | 1097 | −11.484 | −10.894 | 38.904 | 1.00 | 37.55 | O |
| ATOM | 1613 | C | SER | A | 1097 | −9.811 | −7.525 | 38.683 | 1.00 | 38.16 | C |
| ATOM | 1614 | O | SER | A | 1097 | −9.557 | −6.978 | 37.645 | 1.00 | 39.67 | O |
| ATOM | 1615 | N | ALA | A | 1098 | −9.974 | −6.852 | 39.809 | 1.00 | 36.10 | N |
| ATOM | 1616 | CA | ALA | A | 1098 | −9.805 | −5.417 | 39.885 | 1.00 | 36.27 | C |
| ATOM | 1617 | CB | ALA | A | 1098 | −10.192 | −4.906 | 41.274 | 1.00 | 34.17 | C |
| ATOM | 1618 | C | ALA | A | 1098 | −8.389 | −4.960 | 39.529 | 1.00 | 38.85 | C |
| ATOM | 1619 | O | ALA | A | 1098 | −8.212 | −3.959 | 38.872 | 1.00 | 39.31 | O |
| ATOM | 1620 | N | LEU | A | 1099 | −7.383 | −5.742 | 39.925 | 1.00 | 38.36 | N |
| ATOM | 1621 | CA | LEU | A | 1099 | −5.997 | −5.504 | 39.520 | 1.00 | 40.67 | C |
| ATOM | 1622 | CB | LEU | A | 1099 | −5.040 | −6.441 | 40.261 | 1.00 | 36.42 | C |
| ATOM | 1623 | CG | LEU | A | 1099 | −3.560 | −6.059 | 40.195 | 1.00 | 28.26 | C |
| ATOM | 1624 | CD1 | LEU | A | 1099 | −3.310 | −4.754 | 40.936 | 1.00 | 26.69 | C |
| ATOM | 1625 | CD2 | LEU | A | 1099 | −2.693 | −7.174 | 40.753 | 1.00 | 29.94 | C |
| ATOM | 1626 | C | LEU | A | 1099 | −5.803 | −5.638 | 38.011 | 1.00 | 39.62 | C |
| ATOM | 1627 | O | LEU | A | 1099 | −5.208 | −4.786 | 37.388 | 1.00 | 40.04 | O |
| ATOM | 1628 | N | ILE | A | 1100 | −6.375 | −6.708 | 37.436 | 1.00 | 35.69 | N |
| ATOM | 1629 | CA | ILE | A | 1100 | −6.428 | −6.945 | 35.989 | 1.00 | 31.28 | C |
| ATOM | 1630 | CB | ILE | A | 1100 | −7.078 | −8.289 | 35.671 | 1.00 | 28.65 | C |
| ATOM | 1631 | CG1 | ILE | A | 1100 | −6.273 | −9.411 | 36.321 | 1.00 | 32.68 | C |
| ATOM | 1632 | CD1 | ILE | A | 1100 | −6.814 | −10.790 | 36.037 | 1.00 | 36.39 | C |
| ATOM | 1633 | CG2 | ILE | A | 1100 | −7.171 | −8.496 | 34.166 | 1.00 | 33.00 | C |
| ATOM | 1634 | C | ILE | A | 1100 | −7.149 | −5.837 | 35.226 | 1.00 | 30.80 | C |
| ATOM | 1635 | O | ILE | A | 1100 | −6.723 | −5.395 | 34.196 | 1.00 | 35.49 | O |
| ATOM | 1636 | N | ASN | A | 1101 | −8.202 | −5.318 | 35.796 | 1.00 | 34.38 | N |
| ATOM | 1637 | CA | ASN | A | 1101 | −8.891 | −4.156 | 35.267 | 1.00 | 36.01 | C |
| ATOM | 1638 | CB | ASN | A | 1101 | −10.067 | −3.731 | 36.152 | 1.00 | 41.66 | C |
| ATOM | 1639 | CG | ASN | A | 1101 | −11.040 | −2.812 | 35.422 | 1.00 | 43.14 | C |
| ATOM | 1640 | OD1 | ASN | A | 1101 | −10.692 | −1.691 | 35.041 | 1.00 | 43.21 | O |
| ATOM | 1641 | ND2 | ASN | A | 1101 | −12.265 | −3.283 | 35.227 | 1.00 | 39.98 | N |
| ATOM | 1642 | C | ASN | A | 1101 | −7.944 | −2.980 | 35.039 | 1.00 | 36.43 | C |
| ATOM | 1643 | O | ASN | A | 1101 | −7.891 | −2.429 | 33.979 | 1.00 | 36.83 | O |
| ATOM | 1644 | N | MET | A | 1102 | −7.187 | −2.609 | 36.057 | 1.00 | 40.66 | N |
| ATOM | 1645 | CA | MET | A | 1102 | −6.181 | −1.574 | 35.932 | 1.00 | 37.01 | C |
| ATOM | 1646 | CB | MET | A | 1102 | −5.547 | −1.267 | 37.289 | 1.00 | 35.11 | C |
| ATOM | 1647 | CG | MET | A | 1102 | −6.512 | −0.752 | 38.333 | 1.00 | 41.23 | C |
| ATOM | 1648 | SD | MET | A | 1102 | −5.662 | −0.325 | 39.865 | 1.00 | 50.94 | S |
| ATOM | 1649 | CE | MET | A | 1102 | −4.725 | 1.101 | 39.328 | 1.00 | 39.14 | C |
| ATOM | 1650 | C | MET | A | 1102 | −5.070 | −1.926 | 34.919 | 1.00 | 36.32 | C |
| ATOM | 1651 | O | MET | A | 1102 | −4.678 | −1.116 | 34.132 | 1.00 | 35.24 | O |
| ATOM | 1652 | N | VAL | A | 1103 | −4.624 | −3.160 | 34.873 | 1.00 | 33.40 | N |
| ATOM | 1653 | CA | VAL | A | 1103 | −3.677 | −3.600 | 33.860 | 1.00 | 32.33 | C |
| ATOM | 1654 | CB | VAL | A | 1103 | −3.276 | −5.083 | 34.055 | 1.00 | 38.29 | C |
| ATOM | 1655 | CG1 | VAL | A | 1103 | −2.517 | −5.616 | 32.844 | 1.00 | 47.63 | C |
| ATOM | 1656 | CG2 | VAL | A | 1103 | −2.439 | −5.232 | 35.308 | 1.00 | 35.52 | C |
| ATOM | 1657 | C | VAL | A | 1103 | −4.161 | −3.349 | 32.446 | 1.00 | 35.30 | C |
| ATOM | 1658 | O | VAL | A | 1103 | −3.412 | −2.936 | 31.599 | 1.00 | 37.34 | O |
| ATOM | 1659 | N | PHE | A | 1104 | −5.421 | −3.611 | 32.179 | 1.00 | 31.97 | N |
| ATOM | 1660 | CA | PHE | A | 1104 | −5.991 | −3.386 | 30.853 | 1.00 | 30.66 | C |
| ATOM | 1661 | CB | PHE | A | 1104 | −7.330 | −4.091 | 30.675 | 1.00 | 38.08 | C |
| ATOM | 1662 | CG | PHE | A | 1104 | −7.196 | −5.576 | 30.499 | 1.00 | 44.68 | C |
| ATOM | 1663 | CD2 | PHE | A | 1104 | −8.310 | −6.382 | 30.457 | 1.00 | 38.94 | C |
| ATOM | 1664 | CE2 | PHE | A | 1104 | −8.195 | −7.751 | 30.299 | 1.00 | 42.84 | C |
| ATOM | 1665 | CZ | PHE | A | 1104 | −6.947 | −8.330 | 30.167 | 1.00 | 49.30 | C |
| ATOM | 1666 | CE1 | PHE | A | 1104 | −5.818 | −7.534 | 30.197 | 1.00 | 58.57 | C |
| ATOM | 1667 | CD1 | PHE | A | 1104 | −5.943 | −6.165 | 30.363 | 1.00 | 54.26 | C |
| ATOM | 1668 | C | PHE | A | 1104 | −6.018 | −1.911 | 30.479 | 1.00 | 33.32 | C |
| ATOM | 1669 | O | PHE | A | 1104 | −5.675 | −1.551 | 29.362 | 1.00 | 43.06 | O |
| ATOM | 1670 | N | GLN | A | 1105 | −6.395 | −1.058 | 31.431 | 1.00 | 28.37 | N |
| ATOM | 1671 | CA | GLN | A | 1105 | −6.432 | 0.361 | 31.194 | 1.00 | 30.04 | C |
| ATOM | 1672 | CB | GLN | A | 1105 | −7.367 | 1.031 | 32.197 | 1.00 | 30.40 | C |
| ATOM | 1673 | CG | GLN | A | 1105 | −7.783 | 2.424 | 31.788 | 1.00 | 32.77 | C |
| ATOM | 1674 | CD | GLN | A | 1105 | −8.725 | 3.054 | 32.781 | 1.00 | 32.39 | C |
| ATOM | 1675 | OE1 | GLN | A | 1105 | −9.049 | 2.457 | 33.807 | 1.00 | 38.96 | O |
| ATOM | 1676 | NE2 | GLN | A | 1105 | −9.172 | 4.270 | 32.486 | 1.00 | 35.28 | N |
| ATOM | 1677 | C | GLN | A | 1105 | −5.048 | 1.059 | 31.201 | 1.00 | 38.81 | C |
| ATOM | 1678 | O | GLN | A | 1105 | −4.798 | 1.926 | 30.374 | 1.00 | 37.75 | O |
| ATOM | 1679 | N | MET | A | 1106 | −4.190 | 0.752 | 32.209 | 1.00 | 35.51 | N |
| ATOM | 1680 | CA | MET | A | 1106 | −2.903 | 1.459 | 32.387 | 1.00 | 34.62 | C |
| ATOM | 1681 | CB | MET | A | 1106 | −2.625 | 1.708 | 33.866 | 1.00 | 35.37 | C |
| ATOM | 1682 | CG | MET | A | 1106 | −3.813 | 2.046 | 34.712 | 1.00 | 40.98 | C |
| ATOM | 1683 | SD | MET | A | 1106 | −3.176 | 2.492 | 36.326 | 1.00 | 50.62 | S |
| ATOM | 1684 | CE | MET | A | 1106 | −2.310 | 3.993 | 35.865 | 1.00 | 40.31 | C |
| ATOM | 1685 | C | MET | A | 1106 | −1.665 | 0.754 | 31.839 | 1.00 | 39.05 | C |
| ATOM | 1686 | O | MET | A | 1106 | −0.672 | 1.386 | 31.543 | 1.00 | 37.61 | O |
| ATOM | 1687 | N | GLY | A | 1107 | −1.676 | −0.555 | 31.872 | 1.00 | 39.12 | N |
| ATOM | 1688 | CA | GLY | A | 1107 | −0.533 | −1.369 | 31.582 | 1.00 | 38.62 | C |
| ATOM | 1689 | C | GLY | A | 1107 | 0.130 | −1.807 | 32.842 | 1.00 | 37.51 | C |
| ATOM | 1690 | O | GLY | A | 1107 | −0.094 | −1.223 | 33.871 | 1.00 | 39.11 | O |
| ATOM | 1691 | N | GLU | A | 1108 | 0.985 | −2.806 | 32.769 | 1.00 | 44.64 | N |
| ATOM | 1692 | CA | GLU | A | 1108 | 1.761 | −3.264 | 33.904 | 1.00 | 48.80 | C |
| ATOM | 1693 | CB | GLU | A | 1108 | 2.598 | −4.472 | 33.487 | 1.00 | 51.46 | C |
| ATOM | 1694 | CG | GLU | A | 1108 | 1.781 | −5.771 | 33.428 | 1.00 | 53.95 | C |
| ATOM | 1695 | CD | GLU | A | 1108 | 2.354 | −6.782 | 32.448 | 1.00 | 73.60 | C |
| ATOM | 1696 | OE1 | GLU | A | 1108 | 3.594 | −6.937 | 32.470 | 1.00 | 83.60 | O |
| ATOM | 1697 | OE2 | GLU | A | 1108 | 1.597 | −7.426 | 31.669 | 1.00 | 63.75 | O |
| ATOM | 1698 | C | GLU | A | 1108 | 2.660 | −2.164 | 34.516 | 1.00 | 49.07 | C |
| ATOM | 1699 | O | GLU | A | 1108 | 2.744 | −1.990 | 35.716 | 1.00 | 46.40 | O |
| ATOM | 1700 | N | THR | A | 1109 | 3.351 | −1.437 | 33.668 | 1.00 | 47.54 | N |
| ATOM | 1701 | CA | THR | A | 1109 | 4.220 | −0.355 | 34.093 | 1.00 | 48.25 | C |
| ATOM | 1702 | CB | THR | A | 1109 | 4.975 | 0.240 | 32.892 | 1.00 | 60.45 | C |
| ATOM | 1703 | OG1 | THR | A | 1109 | 5.673 | −0.806 | 32.201 | 1.00 | 59.42 | O |
| ATOM | 1704 | CG2 | THR | A | 1109 | 5.966 | 1.305 | 33.351 | 1.00 | 66.84 | C |
| ATOM | 1705 | C | THR | A | 1109 | 3.465 | 0.770 | 34.823 | 1.00 | 46.51 | C |
| ATOM | 1706 | O | THR | A | 1109 | 3.917 | 1.293 | 35.831 | 1.00 | 48.40 | O |
| ATOM | 1707 | N | GLY | A | 1110 | 2.274 | 1.090 | 34.323 | 1.00 | 46.48 | N |
| ATOM | 1708 | CA | GLY | A | 1110 | 1.396 | 2.076 | 34.893 | 1.00 | 45.91 | C |
| ATOM | 1709 | C | GLY | A | 1110 | 0.838 | 1.731 | 36.244 | 1.00 | 47.04 | C |
| ATOM | 1710 | O | GLY | A | 1110 | 0.795 | 2.580 | 37.108 | 1.00 | 44.72 | O |
| ATOM | 1711 | N | VAL | A | 1111 | 0.421 | 0.479 | 36.428 | 1.00 | 47.68 | N |
| ATOM | 1712 | CA | VAL | A | 1111 | −0.149 | −0.001 | 37.689 | 1.00 | 46.14 | C |
| ATOM | 1713 | CB | VAL | A | 1111 | −0.876 | −1.350 | 37.504 | 1.00 | 42.12 | C |
| ATOM | 1714 | CG2 | VAL | A | 1111 | −1.983 | −1.213 | 36.480 | 1.00 | 43.45 | C |
| ATOM | 1715 | CG1 | VAL | A | 1111 | −1.431 | −1.856 | 38.831 | 1.00 | 34.83 | C |
| ATOM | 1716 | C | VAL | A | 1111 | 0.937 | −0.169 | 38.755 | 1.00 | 43.73 | C |
| ATOM | 1717 | O | VAL | A | 1111 | 0.747 | 0.128 | 39.913 | 1.00 | 40.73 | O |
| ATOM | 1718 | N | ALA | A | 1112 | 2.117 | −0.570 | 38.311 | 1.00 | 47.51 | N |
| ATOM | 1719 | CA | ALA | A | 1112 | 3.304 | −0.679 | 39.154 | 1.00 | 49.21 | C |
| ATOM | 1720 | CB | ALA | A | 1112 | 4.490 | −1.174 | 38.334 | 1.00 | 47.44 | C |
| ATOM | 1721 | C | ALA | A | 1112 | 3.655 | 0.642 | 39.865 | 1.00 | 43.66 | C |
| ATOM | 1722 | O | ALA | A | 1112 | 3.994 | 0.666 | 41.027 | 1.00 | 47.62 | O |
| ATOM | 1723 | N | GLY | A | 1113 | 3.525 | 1.742 | 39.169 | 1.00 | 34.52 | N |
| ATOM | 1724 | CA | GLY | A | 1113 | 3.698 | 3.049 | 39.760 | 1.00 | 40.34 | C |
| ATOM | 1725 | C | GLY | A | 1113 | 2.980 | 3.398 | 41.072 | 1.00 | 43.97 | C |
| ATOM | 1726 | O | GLY | A | 1113 | 3.339 | 4.357 | 41.725 | 1.00 | 49.65 | O |
| ATOM | 1727 | N | PHE | A | 1114 | 1.956 | 2.649 | 41.444 | 1.00 | 50.92 | N |
| ATOM | 1728 | CA | PHE | A | 1114 | 1.203 | 2.870 | 42.671 | 1.00 | 52.14 | C |
| ATOM | 1729 | CB | PHE | A | 1114 | −0.217 | 2.346 | 42.499 | 1.00 | 48.77 | C |
| ATOM | 1730 | CG | PHE | A | 1114 | −1.064 | 3.179 | 41.597 | 1.00 | 48.63 | C |
| ATOM | 1731 | CD2 | PHE | A | 1114 | −2.023 | 4.021 | 42.123 | 1.00 | 45.96 | C |
| ATOM | 1732 | CE2 | PHE | A | 1114 | −2.810 | 4.778 | 41.302 | 1.00 | 42.36 | C |
| ATOM | 1733 | CZ | PHE | A | 1114 | −2.649 | 4.712 | 39.934 | 1.00 | 35.75 | C |
| ATOM | 1734 | CE1 | PHE | A | 1114 | −1.700 | 3.880 | 39.396 | 1.00 | 37.73 | C |
| ATOM | 1735 | CD1 | PHE | A | 1114 | −0.915 | 3.113 | 40.223 | 1.00 | 42.50 | C |
| ATOM | 1736 | C | PHE | A | 1114 | 1.826 | 2.149 | 43.872 | 1.00 | 60.74 | C |
| ATOM | 1737 | O | PHE | A | 1114 | 1.327 | 1.120 | 44.288 | 1.00 | 62.16 | O |
| ATOM | 1738 | N | THR | A | 1115 | 2.965 | 2.620 | 44.370 | 1.00 | 64.09 | N |
| ATOM | 1739 | CA | THR | A | 1115 | 3.734 | 1.856 | 45.355 | 1.00 | 63.33 | C |
| ATOM | 1740 | CB | THR | A | 1115 | 5.104 | 2.507 | 45.629 | 1.00 | 56.42 | C |
| ATOM | 1741 | OG1 | THR | A | 1115 | 5.847 | 2.587 | 44.405 | 1.00 | 48.62 | O |
| ATOM | 1742 | CG2 | THR | A | 1115 | 5.890 | 1.689 | 46.642 | 1.00 | 53.82 | C |
| ATOM | 1743 | C | THR | A | 1115 | 2.986 | 1.630 | 46.685 | 1.00 | 63.02 | C |
| ATOM | 1744 | O | THR | A | 1115 | 2.867 | 0.506 | 47.165 | 1.00 | 55.24 | O |
| ATOM | 1745 | N | ASN | A | 1116 | 2.533 | 2.729 | 47.286 | 1.00 | 63.66 | N |
| ATOM | 1746 | CA | ASN | A | 1116 | 1.880 | 2.703 | 48.592 | 1.00 | 64.21 | C |
| ATOM | 1747 | CB | ASN | A | 1116 | 1.815 | 4.111 | 49.183 | 1.00 | 65.74 | C |
| ATOM | 1748 | CG | ASN | A | 1116 | 3.186 | 4.709 | 49.399 | 1.00 | 64.93 | C |
| ATOM | 1749 | OD1 | ASN | A | 1116 | 4.150 | 3.992 | 49.673 | 1.00 | 64.64 | O |
| ATOM | 1750 | ND2 | ASN | A | 1116 | 3.284 | 6.028 | 49.276 | 1.00 | 63.27 | N |
| ATOM | 1751 | C | ASN | A | 1116 | 0.464 | 2.057 | 48.587 | 1.00 | 67.46 | C |
| ATOM | 1752 | O | ASN | A | 1116 | 0.055 | 1.371 | 49.513 | 1.00 | 67.04 | O |
| ATOM | 1753 | N | SER | A | 1117 | −0.271 | 2.236 | 47.503 | 1.00 | 66.16 | N |
| ATOM | 1754 | CA | SER | A | 1117 | −1.574 | 1.610 | 47.352 | 1.00 | 58.86 | C |
| ATOM | 1755 | CB | SER | A | 1117 | −2.250 | 2.094 | 46.073 | 1.00 | 55.90 | C |
| ATOM | 1756 | OG | SER | A | 1117 | −2.316 | 3.506 | 46.042 | 1.00 | 58.92 | O |
| ATOM | 1757 | C | SER | A | 1117 | −1.481 | 0.081 | 47.333 | 1.00 | 59.29 | C |
| ATOM | 1758 | O | SER | A | 1117 | −2.234 | −0.622 | 47.975 | 1.00 | 57.01 | O |
| ATOM | 1759 | N | LEU | A | 1118 | −0.505 | −0.420 | 46.598 | 1.00 | 54.71 | N |
| ATOM | 1760 | CA | LEU | A | 1118 | −0.281 | −1.831 | 46.492 | 1.00 | 52.17 | C |
| ATOM | 1761 | CB | LEU | A | 1118 | 0.686 | −2.134 | 45.345 | 1.00 | 49.05 | C |
| ATOM | 1762 | CG | LEU | A | 1118 | 0.261 | −1.848 | 43.906 | 1.00 | 49.00 | C |
| ATOM | 1763 | CD1 | LEU | A | 1118 | 1.442 | −2.068 | 42.977 | 1.00 | 45.57 | C |
| ATOM | 1764 | CD2 | LEU | A | 1118 | −0.918 | −2.712 | 43.483 | 1.00 | 48.64 | C |
| ATOM | 1765 | C | LEU | A | 1118 | 0.291 | −2.412 | 47.791 | 1.00 | 64.60 | C |
| ATOM | 1766 | O | LEU | A | 1118 | 0.000 | −3.539 | 48.159 | 1.00 | 65.68 | O |
| ATOM | 1767 | N | ARG | A | 1119 | 1.139 | −1.644 | 48.490 | 1.00 | 67.12 | N |
| ATOM | 1768 | CA | ARG | A | 1119 | 1.630 | −2.079 | 49.809 | 1.00 | 67.55 | C |
| ATOM | 1769 | CB | ARG | A | 1119 | 2.568 | −1.032 | 50.422 | 1.00 | 67.57 | C |
| ATOM | 1770 | CG | ARG | A | 1119 | 3.239 | −1.474 | 51.722 | 1.00 | 56.60 | C |
| ATOM | 1771 | CD | ARG | A | 1119 | 3.794 | −0.292 | 52.514 | 1.00 | 56.91 | C |
| ATOM | 1772 | NE | ARG | A | 1119 | 4.363 | −0.719 | 53.794 | 1.00 | 75.21 | N |
| ATOM | 1773 | CZ | ARG | A | 1119 | 4.091 | −0.161 | 54.974 | 1.00 | 82.51 | C |
| ATOM | 1774 | NH1 | ARG | A | 1119 | 4.661 | −0.632 | 56.076 | 1.00 | 73.53 | N |
| ATOM | 1775 | NH2 | ARG | A | 1119 | 3.253 | 0.866 | 55.060 | 1.00 | 82.87 | N |
| ATOM | 1776 | C | ARG | A | 1119 | 0.451 | −2.336 | 50.737 | 1.00 | 66.28 | C |
| ATOM | 1777 | O | ARG | A | 1119 | 0.272 | −3.444 | 51.207 | 1.00 | 59.86 | O |
| ATOM | 1778 | N | MET | A | 1120 | −0.392 | −1.307 | 50.900 | 1.00 | 63.95 | N |
| ATOM | 1779 | CA | MET | A | 1120 | −1.640 | −1.392 | 51.643 | 1.00 | 59.84 | C |
| ATOM | 1780 | CB | MET | A | 1120 | −2.368 | −0.060 | 51.584 | 1.00 | 54.14 | C |
| ATOM | 1781 | CG | MET | A | 1120 | −1.634 | 1.012 | 52.341 | 1.00 | 53.92 | C |
| ATOM | 1782 | SD | MET | A | 1120 | −2.226 | 2.645 | 51.920 | 1.00 | 67.98 | S |
| ATOM | 1783 | CE | MET | A | 1120 | −1.459 | 3.599 | 53.223 | 1.00 | 69.88 | C |
| ATOM | 1784 | C | MET | A | 1120 | −2.538 | −2.527 | 51.185 | 1.00 | 57.30 | C |
| ATOM | 1785 | O | MET | A | 1120 | −2.910 | −3.352 | 51.985 | 1.00 | 53.93 | O |
| ATOM | 1786 | N | LEU | A | 1121 | −2.802 | −2.637 | 49.891 | 1.00 | 55.00 | N |
| ATOM | 1787 | CA | LEU | A | 1121 | −3.485 | −3.817 | 49.358 | 1.00 | 56.14 | C |
| ATOM | 1788 | CB | LEU | A | 1121 | −3.430 | −3.838 | 47.826 | 1.00 | 52.51 | C |
| ATOM | 1789 | CG | LEU | A | 1121 | −4.285 | −2.828 | 47.052 | 1.00 | 56.51 | C |
| ATOM | 1790 | CD1 | LEU | A | 1121 | −4.109 | −3.007 | 45.546 | 1.00 | 50.59 | C |
| ATOM | 1791 | CD2 | LEU | A | 1121 | −5.749 | −2.945 | 47.440 | 1.00 | 49.53 | C |
| ATOM | 1792 | C | LEU | A | 1121 | −2.905 | −5.122 | 49.913 | 1.00 | 61.56 | C |
| ATOM | 1793 | O | LEU | A | 1121 | −3.601 | −5.989 | 50.411 | 1.00 | 55.89 | O |
| ATOM | 1794 | N | GLN | A | 1122 | −1.595 | −5.257 | 49.817 | 1.00 | 66.58 | N |
| ATOM | 1795 | CA | GLN | A | 1122 | −0.940 | −6.446 | 50.310 | 1.00 | 62.31 | C |
| ATOM | 1796 | CB | GLN | A | 1122 | 0.546 | −6.422 | 49.959 | 1.00 | 60.97 | C |
| ATOM | 1797 | CG | GLN | A | 1122 | 1.239 | −7.746 | 50.208 | 1.00 | 60.06 | C |
| ATOM | 1798 | CD | GLN | A | 1122 | 2.651 | −7.758 | 49.684 | 1.00 | 66.02 | C |
| ATOM | 1799 | OE1 | GLN | A | 1122 | 3.185 | −6.717 | 49.296 | 1.00 | 66.65 | O |
| ATOM | 1800 | NE2 | GLN | A | 1122 | 3.268 | −8.937 | 49.661 | 1.00 | 67.31 | N |
| ATOM | 1801 | C | GLN | A | 1122 | −1.115 | −6.684 | 51.807 | 1.00 | 56.51 | C |
| ATOM | 1802 | O | GLN | A | 1122 | −1.172 | −7.811 | 52.257 | 1.00 | 56.64 | O |
| ATOM | 1803 | N | GLN | A | 1123 | −1.189 | −5.620 | 52.578 | 1.00 | 57.95 | N |
| ATOM | 1804 | CA | GLN | A | 1123 | −1.427 | −5.744 | 54.006 | 1.00 | 61.63 | C |
| ATOM | 1805 | CB | GLN | A | 1123 | −0.697 | −4.640 | 54.775 | 1.00 | 63.30 | C |
| ATOM | 1806 | CG | GLN | A | 1123 | 0.817 | −4.693 | 54.629 | 1.00 | 65.08 | C |
| ATOM | 1807 | CD | GLN | A | 1123 | 1.514 | −3.577 | 55.379 | 1.00 | 68.12 | C |
| ATOM | 1808 | OE1 | GLN | A | 1123 | 0.893 | −2.574 | 55.734 | 1.00 | 68.95 | O |
| ATOM | 1809 | NE2 | GLN | A | 1123 | 2.809 | −3.747 | 55.632 | 1.00 | 63.85 | N |
| ATOM | 1810 | C | GLN | A | 1123 | −2.925 | −5.752 | 54.377 | 1.00 | 62.94 | C |
| ATOM | 1811 | O | GLN | A | 1123 | −3.289 | −5.612 | 55.543 | 1.00 | 60.63 | O |
| ATOM | 1812 | N | LYS | A | 1124 | −3.786 | −5.929 | 53.361 | 1.00 | 63.65 | N |
| ATOM | 1813 | CA | LYS | A | 1124 | −5.216 | −5.997 | 53.549 | 1.00 | 51.76 | C |
| ATOM | 1814 | CB | LYS | A | 1124 | −5.612 | −7.318 | 54.210 | 1.00 | 50.86 | C |
| ATOM | 1815 | CG | LYS | A | 1124 | −5.243 | −8.518 | 53.352 | 1.00 | 51.40 | C |
| ATOM | 1816 | CD | LYS | A | 1124 | −5.732 | −9.826 | 53.939 | 1.00 | 58.92 | C |
| ATOM | 1817 | CE | LYS | A | 1124 | −5.555 | −10.959 | 52.939 | 1.00 | 65.51 | C |
| ATOM | 1818 | NZ | LYS | A | 1124 | −4.134 | −11.110 | 52.516 | 1.00 | 72.26 | N |
| ATOM | 1819 | C | LYS | A | 1124 | −5.816 | −4.784 | 54.265 | 1.00 | 49.31 | C |
| ATOM | 1820 | O | LYS | A | 1124 | −6.679 | −4.901 | 55.116 | 1.00 | 55.50 | O |
| ATOM | 1821 | N | ARG | A | 1125 | −5.290 | −3.621 | 53.930 | 1.00 | 48.57 | N |
| ATOM | 1822 | CA | ARG | A | 1125 | −5.729 | −2.357 | 54.474 | 1.00 | 55.63 | C |
| ATOM | 1823 | CB | ARG | A | 1125 | −4.522 | −1.517 | 54.896 | 1.00 | 63.43 | C |
| ATOM | 1824 | CG | ARG | A | 1125 | −3.729 | −2.169 | 56.029 | 1.00 | 64.61 | C |
| ATOM | 1825 | CD | ARG | A | 1125 | −2.305 | −1.655 | 56.107 | 1.00 | 63.82 | C |
| ATOM | 1826 | NE | ARG | A | 1125 | −2.246 | −0.246 | 56.476 | 1.00 | 72.72 | N |
| ATOM | 1827 | CZ | ARG | A | 1125 | −1.193 | 0.532 | 56.253 | 1.00 | 78.11 | C |
| ATOM | 1828 | NH1 | ARG | A | 1125 | −0.117 | 0.036 | 55.656 | 1.00 | 69.76 | N |
| ATOM | 1829 | NH2 | ARG | A | 1125 | −1.216 | 1.807 | 56.620 | 1.00 | 88.19 | N |
| ATOM | 1830 | C | ARG | A | 1125 | −6.649 | −1.610 | 53.504 | 1.00 | 63.27 | C |
| ATOM | 1831 | O | ARG | A | 1125 | −6.289 | −0.562 | 52.986 | 1.00 | 60.71 | O |
| ATOM | 1832 | N | TRP | A | 1126 | −7.772 | −2.246 | 53.147 | 1.00 | 61.78 | N |
| ATOM | 1833 | CA | TRP | A | 1126 | −8.536 | −1.899 | 51.943 | 1.00 | 54.01 | C |
| ATOM | 1834 | CB | TRP | A | 1126 | −9.737 | −2.837 | 51.770 | 1.00 | 51.36 | C |
| ATOM | 1835 | CG | TRP | A | 1126 | −9.451 | −4.296 | 51.961 | 1.00 | 53.09 | C |
| ATOM | 1836 | CD1 | TRP | A | 1126 | −10.009 | −5.121 | 52.896 | 1.00 | 49.29 | C |
| ATOM | 1837 | NE1 | TRP | A | 1126 | −9.508 | −6.392 | 52.764 | 1.00 | 46.22 | N |
| ATOM | 1838 | CE2 | TRP | A | 1126 | −8.608 | −6.411 | 51.731 | 1.00 | 49.71 | C |
| ATOM | 1839 | CD2 | TRP | A | 1126 | −8.547 | −5.107 | 51.199 | 1.00 | 50.10 | C |
| ATOM | 1840 | CE3 | TRP | A | 1126 | −7.693 | −4.859 | 50.120 | 1.00 | 44.49 | C |
| ATOM | 1841 | CZ3 | TRP | A | 1126 | −6.937 | −5.906 | 49.618 | 1.00 | 46.34 | C |
| ATOM | 1842 | CH2 | TRP | A | 1126 | −7.021 | −7.190 | 50.171 | 1.00 | 47.40 | C |
| ATOM | 1843 | CZ2 | TRP | A | 1126 | −7.848 | −7.461 | 51.224 | 1.00 | 52.18 | C |
| ATOM | 1844 | C | TRP | A | 1126 | −9.072 | −0.475 | 51.926 | 1.00 | 55.28 | C |
| ATOM | 1845 | O | TRP | A | 1126 | −9.035 | 0.207 | 50.912 | 1.00 | 51.28 | O |
| ATOM | 1846 | N | ASP | A | 1127 | −9.572 | −0.039 | 53.068 | 1.00 | 62.49 | N |
| ATOM | 1847 | CA | ASP | A | 1127 | −10.030 | 1.336 | 53.257 | 1.00 | 72.81 | C |
| ATOM | 1848 | CB | ASP | A | 1127 | −10.642 | 1.528 | 54.643 | 1.00 | 84.26 | C |
| ATOM | 1849 | CG | ASP | A | 1127 | −12.011 | 0.891 | 54.749 | 1.00 | 92.55 | C |
| ATOM | 1850 | OD1 | ASP | A | 1127 | −12.645 | 0.692 | 53.690 | 1.00 | 85.17 | O |
| ATOM | 1851 | OD2 | ASP | A | 1127 | −12.454 | 0.587 | 55.874 | 1.00 | 102.80 | O |
| ATOM | 1852 | C | ASP | A | 1127 | −8.979 | 2.379 | 52.967 | 1.00 | 70.19 | C |
| ATOM | 1853 | O | ASP | A | 1127 | −9.266 | 3.358 | 52.317 | 1.00 | 73.07 | O |
| ATOM | 1854 | N | GLU | A | 1128 | −7.763 | 2.162 | 53.469 | 1.00 | 70.05 | N |
| ATOM | 1855 | CA | GLU | A | 1128 | −6.654 | 3.100 | 53.247 | 1.00 | 71.25 | C |
| ATOM | 1856 | CB | GLU | A | 1128 | −5.544 | 2.877 | 54.278 | 1.00 | 76.64 | C |
| ATOM | 1857 | CG | GLU | A | 1128 | −5.974 | 3.148 | 55.712 | 1.00 | 86.94 | C |
| ATOM | 1858 | CD | GLU | A | 1128 | −4.877 | 2.855 | 56.715 | 1.00 | 92.12 | C |
| ATOM | 1859 | OE1 | GLU | A | 1128 | −3.868 | 2.227 | 56.325 | 1.00 | 82.51 | O |
| ATOM | 1860 | OE2 | GLU | A | 1128 | −5.026 | 3.254 | 57.890 | 1.00 | 89.42 | O |
| ATOM | 1861 | C | GLU | A | 1128 | −6.084 | 3.012 | 51.815 | 1.00 | 63.42 | C |
| ATOM | 1862 | O | GLU | A | 1128 | −5.638 | 3.991 | 51.243 | 1.00 | 59.58 | O |
| ATOM | 1863 | N | ALA | A | 1129 | −6.119 | 1.808 | 51.238 | 1.00 | 58.18 | N |
| ATOM | 1864 | CA | ALA | A | 1129 | −5.668 | 1.589 | 49.888 | 1.00 | 51.97 | C |
| ATOM | 1865 | CB | ALA | A | 1129 | −5.639 | 0.099 | 49.575 | 1.00 | 56.37 | C |
| ATOM | 1866 | C | ALA | A | 1129 | −6.529 | 2.326 | 48.872 | 1.00 | 60.10 | C |
| ATOM | 1867 | O | ALA | A | 1129 | −6.044 | 2.902 | 47.915 | 1.00 | 60.34 | O |
| ATOM | 1868 | N | ALA | A | 1130 | −7.823 | 2.378 | 49.150 | 1.00 | 63.06 | N |
| ATOM | 1869 | CA | ALA | A | 1130 | −8.786 | 3.090 | 48.333 | 1.00 | 52.94 | C |
| ATOM | 1870 | CB | ALA | A | 1130 | −10.191 | 2.805 | 48.815 | 1.00 | 61.16 | C |
| ATOM | 1871 | C | ALA | A | 1130 | −8.531 | 4.590 | 48.307 | 1.00 | 56.73 | C |
| ATOM | 1872 | O | ALA | A | 1130 | −8.688 | 5.236 | 47.292 | 1.00 | 62.84 | O |
| ATOM | 1873 | N | VAL | A | 1131 | −8.172 | 5.163 | 49.444 | 1.00 | 58.29 | N |
| ATOM | 1874 | CA | VAL | A | 1131 | −7.893 | 6.599 | 49.525 | 1.00 | 59.28 | C |
| ATOM | 1875 | CB | VAL | A | 1131 | −7.513 | 7.012 | 50.968 | 1.00 | 58.90 | C |
| ATOM | 1876 | CG1 | VAL | A | 1131 | −7.193 | 8.501 | 51.044 | 1.00 | 57.91 | C |
| ATOM | 1877 | CG2 | VAL | A | 1131 | −8.632 | 6.657 | 51.931 | 1.00 | 69.73 | C |
| ATOM | 1878 | C | VAL | A | 1131 | −6.768 | 7.014 | 48.577 | 1.00 | 57.14 | C |
| ATOM | 1879 | O | VAL | A | 1131 | −6.834 | 8.057 | 47.952 | 1.00 | 56.65 | O |
| ATOM | 1880 | N | ASN | A | 1132 | −5.714 | 6.199 | 48.537 | 1.00 | 55.45 | N |
| ATOM | 1881 | CA | ASN | A | 1132 | −4.517 | 6.491 | 47.762 | 1.00 | 58.97 | C |
| ATOM | 1882 | CB | ASN | A | 1132 | −3.353 | 5.615 | 48.220 | 1.00 | 61.88 | C |
| ATOM | 1883 | CG | ASN | A | 1132 | −2.782 | 6.059 | 49.544 | 1.00 | 70.36 | C |
| ATOM | 1884 | OD1 | ASN | A | 1132 | −2.895 | 7.227 | 49.920 | 1.00 | 74.30 | O |
| ATOM | 1885 | ND2 | ASN | A | 1132 | −2.158 | 5.133 | 50.260 | 1.00 | 73.13 | N |
| ATOM | 1886 | C | ASN | A | 1132 | −4.728 | 6.330 | 46.250 | 1.00 | 57.41 | C |
| ATOM | 1887 | O | ASN | A | 1132 | −4.240 | 7.115 | 45.435 | 1.00 | 57.69 | O |
| ATOM | 1888 | N | LEU | A | 1133 | −5.521 | 5.336 | 45.887 | 1.00 | 53.60 | N |
| ATOM | 1889 | CA | LEU | A | 1133 | −5.864 | 5.093 | 44.498 | 1.00 | 48.36 | C |
| ATOM | 1890 | CB | LEU | A | 1133 | −6.728 | 3.840 | 44.369 | 1.00 | 47.25 | C |
| ATOM | 1891 | CG | LEU | A | 1133 | −5.955 | 2.546 | 44.626 | 1.00 | 46.33 | C |
| ATOM | 1892 | CD1 | LEU | A | 1133 | −6.881 | 1.347 | 44.602 | 1.00 | 44.81 | C |
| ATOM | 1893 | CD2 | LEU | A | 1133 | −4.846 | 2.384 | 43.601 | 1.00 | 44.89 | C |
| ATOM | 1894 | C | LEU | A | 1133 | −6.518 | 6.301 | 43.797 | 1.00 | 51.26 | C |
| ATOM | 1895 | O | LEU | A | 1133 | −6.256 | 6.560 | 42.637 | 1.00 | 48.97 | O |
| ATOM | 1896 | N | ALA | A | 1134 | −7.365 | 7.043 | 44.491 | 1.00 | 47.91 | N |
| ATOM | 1897 | CA | ALA | A | 1134 | −8.009 | 8.208 | 43.886 | 1.00 | 49.71 | C |
| ATOM | 1898 | CB | ALA | A | 1134 | −9.059 | 8.774 | 44.831 | 1.00 | 55.24 | C |
| ATOM | 1899 | C | ALA | A | 1134 | −7.025 | 9.322 | 43.450 | 1.00 | 57.69 | C |
| ATOM | 1900 | O | ALA | A | 1134 | −7.142 | 9.954 | 42.384 | 1.00 | 68.21 | O |
| ATOM | 1901 | N | LYS | A | 1135 | −6.089 | 9.615 | 44.327 | 1.00 | 52.25 | N |
| ATOM | 1902 | CA | LYS | A | 1135 | −5.170 | 10.713 | 44.144 | 1.00 | 52.32 | C |
| ATOM | 1903 | CB | LYS | A | 1135 | −4.191 | 10.776 | 45.319 | 1.00 | 56.64 | C |
| ATOM | 1904 | CG | LYS | A | 1135 | −4.857 | 11.150 | 46.632 | 1.00 | 56.67 | C |
| ATOM | 1905 | CD | LYS | A | 1135 | −3.960 | 10.874 | 47.823 | 1.00 | 58.10 | C |
| ATOM | 1906 | CE | LYS | A | 1135 | −4.699 | 11.147 | 49.125 | 1.00 | 63.46 | C |
| ATOM | 1907 | NZ | LYS | A | 1135 | −3.976 | 10.607 | 50.309 | 1.00 | 68.52 | N |
| ATOM | 1908 | C | LYS | A | 1135 | −4.407 | 10.699 | 42.803 | 1.00 | 57.24 | C |
| ATOM | 1909 | O | LYS | A | 1135 | −4.008 | 11.734 | 42.296 | 1.00 | 70.11 | O |
| ATOM | 1910 | N | SER | A | 1136 | −4.221 | 9.498 | 42.271 | 1.00 | 58.46 | N |
| ATOM | 1911 | CA | SER | A | 1136 | −3.648 | 9.336 | 40.954 | 1.00 | 65.62 | C |
| ATOM | 1912 | CB | SER | A | 1136 | −3.527 | 7.865 | 40.613 | 1.00 | 63.88 | C |
| ATOM | 1913 | OG | SER | A | 1136 | −4.812 | 7.269 | 40.494 | 1.00 | 58.95 | O |
| ATOM | 1914 | C | SER | A | 1136 | −4.652 | 9.952 | 40.003 | 1.00 | 54.42 | C |
| ATOM | 1915 | O | SER | A | 1136 | −5.856 | 9.915 | 40.253 | 1.00 | 44.19 | O |
| ATOM | 1916 | N | ARG | A | 1137 | −4.161 | 10.525 | 38.915 | 1.00 | 54.75 | N |
| ATOM | 1917 | CA | ARG | A | 1137 | −5.033 | 11.217 | 37.986 | 1.00 | 50.01 | C |
| ATOM | 1918 | CB | ARG | A | 1137 | −4.219 | 12.024 | 36.963 | 1.00 | 48.01 | C |
| ATOM | 1919 | CG | ARG | A | 1137 | −3.061 | 12.843 | 37.499 | 1.00 | 63.11 | C |
| ATOM | 1920 | CD | ARG | A | 1137 | −2.053 | 13.141 | 36.382 | 1.00 | 60.05 | C |
| ATOM | 1921 | NE | ARG | A | 1137 | −0.693 | 13.373 | 36.873 | 1.00 | 58.45 | N |
| ATOM | 1922 | CZ | ARG | A | 1137 | −0.096 | 14.561 | 36.899 | 1.00 | 56.11 | C |
| ATOM | 1923 | NH1 | ARG | A | 1137 | −0.740 | 15.637 | 36.464 | 1.00 | 61.85 | N |
| ATOM | 1924 | NH2 | ARG | A | 1137 | 1.143 | 14.673 | 37.359 | 1.00 | 48.37 | N |
| ATOM | 1925 | C | ARG | A | 1137 | −5.914 | 10.288 | 37.169 | 1.00 | 54.60 | C |
| ATOM | 1926 | O | ARG | A | 1137 | −6.706 | 10.736 | 36.340 | 1.00 | 54.56 | O |
| ATOM | 1927 | N | TRP | A | 1138 | −5.763 | 8.989 | 37.399 | 1.00 | 56.97 | N |
| ATOM | 1928 | CA | TRP | A | 1138 | −6.435 | 7.995 | 36.572 | 1.00 | 48.26 | C |
| ATOM | 1929 | CB | TRP | A | 1138 | −5.960 | 6.603 | 36.984 | 1.00 | 47.17 | C |
| ATOM | 1930 | CG | TRP | A | 1138 | −6.939 | 5.493 | 36.895 | 1.00 | 41.43 | C |
| ATOM | 1931 | CD1 | TRP | A | 1138 | −7.552 | 5.026 | 35.775 | 1.00 | 39.21 | C |
| ATOM | 1932 | NE1 | TRP | A | 1138 | −8.369 | 3.968 | 36.091 | 1.00 | 40.59 | N |
| ATOM | 1933 | CE2 | TRP | A | 1138 | −8.278 | 3.723 | 37.437 | 1.00 | 44.36 | C |
| ATOM | 1934 | CD2 | TRP | A | 1138 | −7.379 | 4.660 | 37.974 | 1.00 | 43.13 | C |
| ATOM | 1935 | CE3 | TRP | A | 1138 | −7.110 | 4.625 | 39.346 | 1.00 | 41.27 | C |
| ATOM | 1936 | CZ3 | TRP | A | 1138 | −7.737 | 3.665 | 40.123 | 1.00 | 37.43 | C |
| ATOM | 1937 | CH2 | TRP | A | 1138 | −8.625 | 2.747 | 39.560 | 1.00 | 39.67 | C |
| ATOM | 1938 | CZ2 | TRP | A | 1138 | −8.909 | 2.758 | 38.222 | 1.00 | 44.46 | C |
| ATOM | 1939 | C | TRP | A | 1138 | −7.880 | 8.290 | 36.898 | 1.00 | 52.52 | C |
| ATOM | 1940 | O | TRP | A | 1138 | −8.748 | 8.368 | 36.052 | 1.00 | 50.43 | O |
| ATOM | 1941 | N | TYR | A | 1139 | −8.084 | 8.529 | 38.175 | 1.00 | 50.61 | N |
| ATOM | 1942 | CA | TYR | A | 1139 | −9.361 | 8.844 | 38.744 | 1.00 | 51.65 | C |
| ATOM | 1943 | CB | TYR | A | 1139 | −9.174 | 9.031 | 40.252 | 1.00 | 52.59 | C |
| ATOM | 1944 | CG | TYR | A | 1139 | −10.431 | 9.195 | 41.073 | 1.00 | 52.31 | C |
| ATOM | 1945 | CD2 | TYR | A | 1139 | −11.007 | 8.107 | 41.715 | 1.00 | 51.10 | C |
| ATOM | 1946 | CE2 | TYR | A | 1139 | −12.138 | 8.253 | 42.487 | 1.00 | 60.06 | C |
| ATOM | 1947 | CZ | TYR | A | 1139 | −12.702 | 9.503 | 42.637 | 1.00 | 69.31 | C |
| ATOM | 1948 | OH | TYR | A | 1139 | −13.832 | 9.662 | 43.405 | 1.00 | 64.16 | O |
| ATOM | 1949 | CE1 | TYR | A | 1139 | −12.140 | 10.602 | 42.020 | 1.00 | 65.04 | C |
| ATOM | 1950 | CD1 | TYR | A | 1139 | −11.009 | 10.446 | 41.252 | 1.00 | 53.69 | C |
| ATOM | 1951 | C | TYR | A | 1139 | −9.954 | 10.106 | 38.132 | 1.00 | 51.04 | C |
| ATOM | 1952 | O | TYR | A | 1139 | −11.132 | 10.188 | 37.879 | 1.00 | 53.82 | O |
| ATOM | 1953 | N | ASN | A | 1140 | −9.140 | 11.123 | 37.973 | 1.00 | 49.20 | N |
| ATOM | 1954 | CA | ASN | A | 1140 | −9.617 | 12.392 | 37.475 | 1.00 | 49.69 | C |
| ATOM | 1955 | CB | ASN | A | 1140 | −8.525 | 13.456 | 37.569 | 1.00 | 47.69 | C |
| ATOM | 1956 | CG | ASN | A | 1140 | −8.243 | 13.870 | 38.992 | 1.00 | 51.82 | C |
| ATOM | 1957 | OD1 | ASN | A | 1140 | −7.322 | 13.357 | 39.631 | 1.00 | 60.44 | O |
| ATOM | 1958 | ND2 | ASN | A | 1140 | −9.042 | 14.799 | 39.505 | 1.00 | 49.28 | N |
| ATOM | 1959 | C | ASN | A | 1140 | −10.100 | 12.278 | 36.037 | 1.00 | 48.76 | C |
| ATOM | 1960 | O | ASN | A | 1140 | −11.150 | 12.787 | 35.673 | 1.00 | 41.15 | O |
| ATOM | 1961 | N | GLN | A | 1141 | −9.321 | 11.603 | 35.214 | 1.00 | 50.86 | N |
| ATOM | 1962 | CA | GLN | A | 1141 | −9.712 | 11.446 | 33.833 | 1.00 | 56.16 | C |
| ATOM | 1963 | CB | GLN | A | 1141 | −8.546 | 11.022 | 32.942 | 1.00 | 54.19 | C |
| ATOM | 1964 | CG | GLN | A | 1141 | −8.842 | 11.278 | 31.469 | 1.00 | 50.20 | C |
| ATOM | 1965 | CD | GLN | A | 1141 | −8.012 | 10.416 | 30.560 | 1.00 | 52.81 | C |
| ATOM | 1966 | OE1 | GLN | A | 1141 | −6.815 | 10.263 | 30.775 | 1.00 | 65.97 | O |
| ATOM | 1967 | NE2 | GLN | A | 1141 | −8.643 | 9.832 | 29.543 | 1.00 | 47.69 | N |
| ATOM | 1968 | C | GLN | A | 1141 | −10.938 | 10.528 | 33.602 | 1.00 | 51.05 | C |
| ATOM | 1969 | O | GLN | A | 1141 | −11.730 | 10.778 | 32.704 | 1.00 | 42.21 | O |
| ATOM | 1970 | N | THR | A | 1142 | −11.070 | 9.440 | 34.387 | 1.00 | 50.32 | N |
| ATOM | 1971 | CA | THR | A | 1142 | −12.178 | 8.477 | 34.191 | 1.00 | 46.69 | C |
| ATOM | 1972 | CB | THR | A | 1142 | −11.731 | 7.181 | 33.470 | 1.00 | 38.53 | C |
| ATOM | 1973 | OG1 | THR | A | 1142 | −10.674 | 6.553 | 34.205 | 1.00 | 45.28 | O |
| ATOM | 1974 | CG2 | THR | A | 1142 | −11.256 | 7.481 | 32.060 | 1.00 | 44.86 | C |
| ATOM | 1975 | C | THR | A | 1142 | −12.792 | 8.105 | 35.529 | 1.00 | 48.57 | C |
| ATOM | 1976 | O | THR | A | 1142 | −12.609 | 7.002 | 36.023 | 1.00 | 48.88 | O |
| ATOM | 1977 | N | PRO | A | 1143 | −13.494 | 9.030 | 36.161 | 1.00 | 46.11 | N |
| ATOM | 1978 | CA | PRO | A | 1143 | −14.020 | 8.853 | 37.486 | 1.00 | 47.83 | C |
| ATOM | 1979 | CB | PRO | A | 1143 | −14.919 | 10.086 | 37.674 | 1.00 | 50.86 | C |
| ATOM | 1980 | CG | PRO | A | 1143 | −14.425 | 11.082 | 36.689 | 1.00 | 47.26 | C |
| ATOM | 1981 | CD | PRO | A | 1143 | −13.998 | 10.256 | 35.527 | 1.00 | 45.18 | C |
| ATOM | 1982 | C | PRO | A | 1143 | −14.835 | 7.602 | 37.692 | 1.00 | 47.22 | C |
| ATOM | 1983 | O | PRO | A | 1143 | −14.669 | 6.950 | 38.693 | 1.00 | 44.08 | O |
| ATOM | 1984 | N | ASN | A | 1144 | −15.779 | 7.340 | 36.795 | 1.00 | 49.13 | N |
| ATOM | 1985 | CA | ASN | A | 1144 | −16.773 | 6.277 | 36.978 | 1.00 | 48.99 | C |
| ATOM | 1986 | CB | ASN | A | 1144 | −17.864 | 6.376 | 35.922 | 1.00 | 53.73 | C |
| ATOM | 1987 | CG | ASN | A | 1144 | −18.746 | 7.596 | 36.110 | 1.00 | 57.78 | C |
| ATOM | 1988 | OD1 | ASN | A | 1144 | −18.771 | 8.199 | 37.186 | 1.00 | 52.43 | O |
| ATOM | 1989 | ND2 | ASN | A | 1144 | −19.484 | 7.963 | 35.065 | 1.00 | 57.20 | N |
| ATOM | 1990 | C | ASN | A | 1144 | −16.155 | 4.881 | 37.061 | 1.00 | 45.30 | C |
| ATOM | 1991 | O | ASN | A | 1144 | −16.462 | 4.119 | 37.955 | 1.00 | 42.51 | O |
| ATOM | 1992 | N | ARG | A | 1145 | −15.283 | 4.576 | 36.093 | 1.00 | 45.85 | N |
| ATOM | 1993 | CA | ARG | A | 1145 | −14.558 | 3.318 | 36.008 | 1.00 | 37.49 | C |
| ATOM | 1994 | CB | ARG | A | 1145 | −13.839 | 3.155 | 34.666 | 1.00 | 33.77 | C |
| ATOM | 1995 | CG | ARG | A | 1145 | −13.208 | 1.776 | 34.494 | 1.00 | 32.83 | C |
| ATOM | 1996 | CD | ARG | A | 1145 | −12.427 | 1.649 | 33.196 | 1.00 | 34.41 | C |
| ATOM | 1997 | NE | ARG | A | 1145 | −11.781 | 0.341 | 33.085 | 1.00 | 34.65 | N |
| ATOM | 1998 | CZ | ARG | A | 1145 | −11.073 | −0.059 | 32.032 | 1.00 | 36.14 | C |
| ATOM | 1999 | NH1 | ARG | A | 1145 | −10.918 | 0.751 | 30.994 | 1.00 | 39.23 | N |
| ATOM | 2000 | NH2 | ARG | A | 1145 | −10.522 | −1.267 | 32.012 | 1.00 | 29.47 | N |
| ATOM | 2001 | C | ARG | A | 1145 | −13.587 | 3.173 | 37.123 | 1.00 | 41.52 | C |
| ATOM | 2002 | O | ARG | A | 1145 | −13.436 | 2.113 | 37.687 | 1.00 | 42.28 | O |
| ATOM | 2003 | N | ALA | A | 1146 | −12.954 | 4.281 | 37.480 | 1.00 | 44.76 | N |
| ATOM | 2004 | CA | ALA | A | 1146 | −12.057 | 4.288 | 38.600 | 1.00 | 37.77 | C |
| ATOM | 2005 | CB | ALA | A | 1146 | −11.303 | 5.610 | 38.668 | 1.00 | 42.44 | C |
| ATOM | 2006 | C | ALA | A | 1146 | −12.781 | 4.026 | 39.899 | 1.00 | 44.07 | C |
| ATOM | 2007 | O | ALA | A | 1146 | −12.317 | 3.288 | 40.724 | 1.00 | 47.80 | O |
| ATOM | 2008 | N | LYS | A | 1147 | −13.920 | 4.662 | 40.098 | 1.00 | 45.49 | N |
| ATOM | 2009 | CA | LYS | A | 1147 | −14.704 | 4.470 | 41.306 | 1.00 | 39.61 | C |
| ATOM | 2010 | CB | LYS | A | 1147 | −15.938 | 5.375 | 41.306 | 1.00 | 46.23 | C |
| ATOM | 2011 | CG | LYS | A | 1147 | −15.668 | 6.837 | 41.625 | 1.00 | 50.03 | C |
| ATOM | 2012 | CD | LYS | A | 1147 | −16.936 | 7.663 | 41.440 | 1.00 | 59.61 | C |
| ATOM | 2013 | CE | LYS | A | 1147 | −16.665 | 9.155 | 41.549 | 1.00 | 64.15 | C |
| ATOM | 2014 | NZ | LYS | A | 1147 | −17.867 | 9.959 | 41.175 | 1.00 | 65.48 | N |
| ATOM | 2015 | C | LYS | A | 1147 | −15.137 | 3.025 | 41.484 | 1.00 | 40.81 | C |
| ATOM | 2016 | O | LYS | A | 1147 | −14.999 | 2.460 | 42.551 | 1.00 | 40.52 | O |
| ATOM | 2017 | N | ARG | A | 1148 | −15.630 | 2.427 | 40.415 | 1.00 | 41.67 | N |
| ATOM | 2018 | CA | ARG | A | 1148 | −16.007 | 1.025 | 40.444 | 1.00 | 43.37 | C |
| ATOM | 2019 | CB | ARG | A | 1148 | −16.700 | 0.613 | 39.141 | 1.00 | 41.30 | C |
| ATOM | 2020 | CG | ARG | A | 1148 | −18.026 | 1.311 | 38.892 | 1.00 | 45.27 | C |
| ATOM | 2021 | CD | ARG | A | 1148 | −18.786 | 0.660 | 37.749 | 1.00 | 46.21 | C |
| ATOM | 2022 | NE | ARG | A | 1148 | −18.123 | 0.857 | 36.464 | 1.00 | 47.43 | N |
| ATOM | 2023 | CZ | ARG | A | 1148 | −18.394 | 1.856 | 35.631 | 1.00 | 47.60 | C |
| ATOM | 2024 | NH1 | ARG | A | 1148 | −19.319 | 2.753 | 35.948 | 1.00 | 51.69 | N |
| ATOM | 2025 | NH2 | ARG | A | 1148 | −17.744 | 1.958 | 34.480 | 1.00 | 40.40 | N |
| ATOM | 2026 | C | ARG | A | 1148 | −14.821 | 0.093 | 40.728 | 1.00 | 42.95 | C |
| ATOM | 2027 | O | ARG | A | 1148 | −14.930 | −0.824 | 41.508 | 1.00 | 41.05 | O |
| ATOM | 2028 | N | VAL | A | 1149 | −13.692 | 0.311 | 40.061 | 1.00 | 45.29 | N |
| ATOM | 2029 | CA | VAL | A | 1149 | −12.508 | −0.507 | 40.260 | 1.00 | 40.92 | C |
| ATOM | 2030 | CB | VAL | A | 1149 | −11.413 | −0.175 | 39.225 | 1.00 | 41.20 | C |
| ATOM | 2031 | CG1 | VAL | A | 1149 | −10.098 | −0.858 | 39.579 | 1.00 | 46.83 | C |
| ATOM | 2032 | CG2 | VAL | A | 1149 | −11.864 | −0.597 | 37.846 | 1.00 | 41.28 | C |
| ATOM | 2033 | C | VAL | A | 1149 | −11.946 | −0.376 | 41.668 | 1.00 | 43.96 | C |
| ATOM | 2034 | O | VAL | A | 1149 | −11.576 | −1.359 | 42.273 | 1.00 | 43.39 | O |
| ATOM | 2035 | N | ILE | A | 1150 | −11.908 | 0.860 | 42.185 | 1.00 | 40.26 | N |
| ATOM | 2036 | CA | ILE | A | 1150 | −11.445 | 1.144 | 43.533 | 1.00 | 39.46 | C |
| ATOM | 2037 | CB | ILE | A | 1150 | −11.338 | 2.652 | 43.806 | 1.00 | 39.24 | C |
| ATOM | 2038 | CG1 | ILE | A | 1150 | −10.228 | 3.265 | 42.960 | 1.00 | 38.36 | C |
| ATOM | 2039 | CD1 | ILE | A | 1150 | −10.154 | 4.772 | 43.072 | 1.00 | 41.23 | C |
| ATOM | 2040 | CG2 | ILE | A | 1150 | −11.046 | 2.911 | 45.277 | 1.00 | 41.57 | C |
| ATOM | 2041 | C | ILE | A | 1150 | −12.350 | 0.515 | 44.556 | 1.00 | 44.18 | C |
| ATOM | 2042 | O | ILE | A | 1150 | −11.894 | −0.085 | 45.498 | 1.00 | 44.52 | O |
| ATOM | 2043 | N | ALA | A | 1151 | −13.657 | 0.635 | 44.338 | 1.00 | 46.20 | N |
| ATOM | 2044 | CA | ALA | A | 1151 | −14.653 | 0.077 | 45.244 | 1.00 | 40.87 | C |
| ATOM | 2045 | CB | ALA | A | 1151 | −16.048 | 0.485 | 44.801 | 1.00 | 30.13 | C |
| ATOM | 2046 | C | ALA | A | 1151 | −14.551 | −1.440 | 45.354 | 1.00 | 45.28 | C |
| ATOM | 2047 | O | ALA | A | 1151 | −14.749 | −1.999 | 46.398 | 1.00 | 48.31 | O |
| ATOM | 2048 | N | THR | A | 1152 | −14.150 | −2.090 | 44.275 | 1.00 | 43.31 | N |
| ATOM | 2049 | CA | THR | A | 1152 | −13.927 | −3.524 | 44.236 | 1.00 | 39.28 | C |
| ATOM | 2050 | CB | THR | A | 1152 | −13.695 | −4.050 | 42.804 | 1.00 | 39.18 | C |
| ATOM | 2051 | OG1 | THR | A | 1152 | −14.761 | −3.606 | 41.957 | 1.00 | 37.47 | O |
| ATOM | 2052 | CG2 | THR | A | 1152 | −13.651 | −5.579 | 42.792 | 1.00 | 33.82 | C |
| ATOM | 2053 | C | THR | A | 1152 | −12.798 | −3.952 | 45.167 | 1.00 | 41.37 | C |
| ATOM | 2054 | O | THR | A | 1152 | −12.895 | −4.940 | 45.848 | 1.00 | 45.40 | O |
| ATOM | 2055 | N | PHE | A | 1153 | −11.748 | −3.144 | 45.240 | 1.00 | 43.58 | N |
| ATOM | 2056 | CA | PHE | A | 1153 | −10.699 | −3.303 | 46.240 | 1.00 | 46.33 | C |
| ATOM | 2057 | CB | PHE | A | 1153 | −9.562 | −2.306 | 46.016 | 1.00 | 46.14 | C |
| ATOM | 2058 | CG | PHE | A | 1153 | −8.681 | −2.655 | 44.850 | 1.00 | 45.15 | C |
| ATOM | 2059 | CD1 | PHE | A | 1153 | −8.165 | −3.935 | 44.710 | 1.00 | 41.12 | C |
| ATOM | 2060 | CE1 | PHE | A | 1153 | −7.356 | −4.260 | 43.635 | 1.00 | 37.16 | C |
| ATOM | 2061 | CZ | PHE | A | 1153 | −7.059 | −3.309 | 42.682 | 1.00 | 38.05 | C |
| ATOM | 2062 | CE2 | PHE | A | 1153 | −7.572 | −2.033 | 42.806 | 1.00 | 43.60 | C |
| ATOM | 2063 | CD2 | PHE | A | 1153 | −8.382 | −1.712 | 43.884 | 1.00 | 43.87 | C |
| ATOM | 2064 | C | PHE | A | 1153 | −11.236 | −3.241 | 47.676 | 1.00 | 46.05 | C |
| ATOM | 2065 | O | PHE | A | 1153 | −10.990 | −4.129 | 48.455 | 1.00 | 53.09 | O |
| ATOM | 2066 | N | ARG | A | 1154 | −11.996 | −2.195 | 48.020 | 1.00 | 40.77 | N |
| ATOM | 2067 | CA | ARG | A | 1154 | −12.587 | −2.081 | 49.348 | 1.00 | 47.11 | C |
| ATOM | 2068 | CB | ARG | A | 1154 | −13.472 | −0.829 | 49.412 | 1.00 | 52.42 | C |
| ATOM | 2069 | CG | ARG | A | 1154 | −12.759 | 0.478 | 49.638 | 1.00 | 60.07 | C |
| ATOM | 2070 | CD | ARG | A | 1154 | −13.753 | 1.543 | 50.071 | 1.00 | 70.72 | C |
| ATOM | 2071 | NE | ARG | A | 1154 | −13.165 | 2.879 | 50.069 | 1.00 | 81.03 | N |
| ATOM | 2072 | CZ | ARG | A | 1154 | −13.771 | 3.962 | 50.545 | 1.00 | 89.94 | C |
| ATOM | 2073 | NH1 | ARG | A | 1154 | −14.984 | 3.863 | 51.071 | 1.00 | 97.10 | N |
| ATOM | 2074 | NH2 | ARG | A | 1154 | −13.162 | 5.141 | 50.499 | 1.00 | 81.31 | N |
| ATOM | 2075 | C | ARG | A | 1154 | −13.446 | −3.217 | 49.862 | 1.00 | 56.40 | C |
| ATOM | 2076 | O | ARG | A | 1154 | −13.245 | −3.714 | 50.948 | 1.00 | 63.37 | O |
| ATOM | 2077 | N | THR | A | 1155 | −14.457 | −3.575 | 49.074 | 1.00 | 51.45 | N |
| ATOM | 2078 | CA | THR | A | 1155 | −15.476 | −4.516 | 49.477 | 1.00 | 43.27 | C |
| ATOM | 2079 | CB | THR | A | 1155 | −16.814 | −4.269 | 48.744 | 1.00 | 48.79 | C |
| ATOM | 2080 | OG1 | THR | A | 1155 | −16.625 | −4.391 | 47.328 | 1.00 | 49.15 | O |
| ATOM | 2081 | CG2 | THR | A | 1155 | −17.349 | −2.881 | 49.064 | 1.00 | 55.78 | C |
| ATOM | 2082 | C | THR | A | 1155 | −15.024 | −5.944 | 49.264 | 1.00 | 46.49 | C |
| ATOM | 2083 | O | THR | A | 1155 | −15.266 | −6.786 | 50.115 | 1.00 | 56.57 | O |
| ATOM | 2084 | N | GLY | A | 1156 | −14.484 | −6.257 | 48.096 | 1.00 | 40.19 | N |
| ATOM | 2085 | CA | GLY | A | 1156 | −14.255 | −7.648 | 47.796 | 1.00 | 40.73 | C |
| ATOM | 2086 | C | GLY | A | 1156 | −15.248 | −8.178 | 46.851 | 1.00 | 50.20 | C |
| ATOM | 2087 | O | GLY | A | 1156 | −14.990 | −9.145 | 46.179 | 1.00 | 54.95 | O |
| ATOM | 2088 | N | THR | A | 1157 | −16.429 | −7.581 | 46.838 | 1.00 | 54.70 | N |
| ATOM | 2089 | CA | THR | A | 1157 | −17.529 | −8.016 | 46.015 | 1.00 | 47.63 | C |
| ATOM | 2090 | CB | THR | A | 1157 | −18.851 | −7.739 | 46.731 | 1.00 | 46.18 | C |
| ATOM | 2091 | OG1 | THR | A | 1157 | −18.825 | −6.403 | 47.251 | 1.00 | 44.36 | O |
| ATOM | 2092 | CG2 | THR | A | 1157 | −19.049 | −8.715 | 47.880 | 1.00 | 53.03 | C |
| ATOM | 2093 | C | THR | A | 1157 | −17.600 | −7.332 | 44.636 | 1.00 | 47.49 | C |
| ATOM | 2094 | O | THR | A | 1157 | −16.892 | −6.394 | 44.331 | 1.00 | 44.19 | O |
| ATOM | 2095 | N | TRP | A | 1158 | −18.568 | −7.802 | 43.837 | 1.00 | 50.38 | N |
| ATOM | 2096 | CA | TRP | A | 1158 | −18.925 | −7.235 | 42.541 | 1.00 | 49.27 | C |
| ATOM | 2097 | CB | TRP | A | 1158 | −19.391 | −8.347 | 41.596 | 1.00 | 36.53 | C |
| ATOM | 2098 | CG | TRP | A | 1158 | −18.360 | −9.392 | 41.363 | 1.00 | 38.28 | C |
| ATOM | 2099 | CD1 | TRP | A | 1158 | −18.315 | −10.639 | 41.910 | 1.00 | 39.54 | C |
| ATOM | 2100 | NE1 | TRP | A | 1158 | −17.204 | −11.313 | 41.463 | 1.00 | 42.79 | N |
| ATOM | 2101 | CE2 | TRP | A | 1158 | −16.505 | −10.496 | 40.613 | 1.00 | 48.89 | C |
| ATOM | 2102 | CD2 | TRP | A | 1158 | −17.205 | −9.276 | 40.529 | 1.00 | 47.90 | C |
| ATOM | 2103 | CE3 | TRP | A | 1158 | −16.695 | −8.260 | 39.715 | 1.00 | 41.92 | C |
| ATOM | 2104 | CZ3 | TRP | A | 1158 | −15.522 | −8.490 | 39.026 | 1.00 | 49.46 | C |
| ATOM | 2105 | CH2 | TRP | A | 1158 | −14.847 | −9.714 | 39.132 | 1.00 | 52.01 | C |
| ATOM | 2106 | CZ2 | TRP | A | 1158 | −15.322 | −10.726 | 39.918 | 1.00 | 47.45 | C |
| ATOM | 2107 | C | TRP | A | 1158 | −20.043 | −6.190 | 42.649 | 1.00 | 50.46 | C |
| ATOM | 2108 | O | TRP | A | 1158 | −20.719 | −5.912 | 41.684 | 1.00 | 41.49 | O |
| ATOM | 2109 | N | ASP | A | 1159 | −20.267 | −5.642 | 43.838 | 1.00 | 50.42 | N |
| ATOM | 2110 | CA | ASP | A | 1159 | −21.388 | −4.733 | 44.063 | 1.00 | 49.32 | C |
| ATOM | 2111 | CB | ASP | A | 1159 | −21.319 | −4.163 | 45.481 | 1.00 | 53.15 | C |
| ATOM | 2112 | CG | ASP | A | 1159 | −21.532 | −5.209 | 46.539 | 1.00 | 54.15 | C |
| ATOM | 2113 | OD1 | ASP | A | 1159 | −21.714 | −6.390 | 46.175 | 1.00 | 55.79 | O |
| ATOM | 2114 | OD2 | ASP | A | 1159 | −21.513 | −4.846 | 47.736 | 1.00 | 50.29 | O |
| ATOM | 2115 | C | ASP | A | 1159 | −21.418 | −3.552 | 43.098 | 1.00 | 48.97 | C |
| ATOM | 2116 | O | ASP | A | 1159 | −22.465 | −3.167 | 42.627 | 1.00 | 54.09 | O |
| ATOM | 2117 | N | ALA | A | 1160 | −20.247 | −2.985 | 42.839 | 1.00 | 47.48 | N |
| ATOM | 2118 | CA | ALA | A | 1160 | −20.070 | −1.884 | 41.916 | 1.00 | 46.40 | C |
| ATOM | 2119 | CB | ALA | A | 1160 | −18.598 | −1.468 | 41.866 | 1.00 | 46.07 | C |
| ATOM | 2120 | C | ALA | A | 1160 | −20.588 | −2.155 | 40.511 | 1.00 | 43.84 | C |
| ATOM | 2121 | O | ALA | A | 1160 | −21.135 | −1.285 | 39.870 | 1.00 | 44.66 | O |
| ATOM | 2122 | N | TYR | A | 1161 | −20.346 | −3.352 | 40.017 | 1.00 | 34.15 | N |
| ATOM | 2123 | CA | TYR | A | 1161 | −20.786 | −3.730 | 38.701 | 1.00 | 38.60 | C |
| ATOM | 2124 | CB | TYR | A | 1161 | −19.760 | −4.644 | 38.038 | 1.00 | 43.52 | C |
| ATOM | 2125 | CG | TYR | A | 1161 | −18.435 | −3.937 | 37.837 | 1.00 | 48.34 | C |
| ATOM | 2126 | CD1 | TYR | A | 1161 | −18.227 | −3.133 | 36.728 | 1.00 | 44.13 | C |
| ATOM | 2127 | CE1 | TYR | A | 1161 | −17.034 | −2.476 | 36.541 | 1.00 | 45.08 | C |
| ATOM | 2128 | CZ | TYR | A | 1161 | −16.021 | −2.605 | 37.469 | 1.00 | 40.39 | C |
| ATOM | 2129 | OH | TYR | A | 1161 | −14.831 | −1.946 | 37.266 | 1.00 | 39.28 | O |
| ATOM | 2130 | CE2 | TYR | A | 1161 | −16.199 | −3.391 | 38.588 | 1.00 | 38.57 | C |
| ATOM | 2131 | CD2 | TYR | A | 1161 | −17.404 | −4.052 | 38.769 | 1.00 | 43.05 | C |
| ATOM | 2132 | C | TYR | A | 1161 | −22.179 | −4.293 | 38.688 | 1.00 | 46.20 | C |
| ATOM | 2133 | O | TYR | A | 1161 | −22.903 | −4.138 | 37.737 | 1.00 | 39.50 | O |
| ATOM | 2134 | N | ASP | A | 224 | −22.526 | −4.989 | 39.766 | 1.00 | 52.82 | N |
| ATOM | 2135 | CA | ASP | A | 224 | −23.835 | −5.620 | 39.944 | 1.00 | 51.74 | C |
| ATOM | 2136 | CB | ASP | A | 224 | −23.887 | −6.471 | 41.225 | 1.00 | 51.63 | C |
| ATOM | 2137 | CG | ASP | A | 224 | −23.263 | −7.856 | 41.043 | 1.00 | 57.69 | C |
| ATOM | 2138 | OD1 | ASP | A | 224 | −22.964 | −8.232 | 39.886 | 1.00 | 57.35 | O |
| ATOM | 2139 | OD2 | ASP | A | 224 | −23.084 | −8.573 | 42.057 | 1.00 | 44.57 | O |
| ATOM | 2140 | C | ASP | A | 224 | −24.998 | −4.624 | 39.918 | 1.00 | 49.12 | C |
| ATOM | 2141 | O | ASP | A | 224 | −26.035 | −4.914 | 39.359 | 1.00 | 52.76 | O |
| ATOM | 2142 | N | ARG | A | 225 | −24.839 | −3.432 | 40.473 | 1.00 | 42.40 | N |
| ATOM | 2143 | CA | ARG | A | 225 | −25.931 | −2.470 | 40.383 | 1.00 | 51.40 | C |
| ATOM | 2144 | CB | ARG | A | 225 | −25.551 | −1.161 | 41.075 | 1.00 | 64.26 | C |
| ATOM | 2145 | CG | ARG | A | 225 | −24.940 | −1.311 | 42.452 | 1.00 | 66.37 | C |
| ATOM | 2146 | CD | ARG | A | 225 | −24.880 | 0.035 | 43.155 | 1.00 | 78.99 | C |
| ATOM | 2147 | NE | ARG | A | 225 | −24.429 | 1.095 | 42.256 | 1.00 | 76.96 | N |
| ATOM | 2148 | CZ | ARG | A | 225 | −24.505 | 2.393 | 42.535 | 1.00 | 87.02 | C |
| ATOM | 2149 | NH1 | ARG | A | 225 | −25.016 | 2.794 | 43.691 | 1.00 | 85.07 | N |
| ATOM | 2150 | NH2 | ARG | A | 225 | −24.074 | 3.291 | 41.658 | 1.00 | 94.10 | N |
| ATOM | 2151 | C | ARG | A | 225 | −26.335 | −2.146 | 38.927 | 1.00 | 51.69 | C |
| ATOM | 2152 | O | ARG | A | 225 | −27.491 | −2.157 | 38.538 | 1.00 | 58.41 | O |
| ATOM | 2153 | N | LEU | A | 226 | −25.323 | −1.913 | 38.121 | 1.00 | 51.69 | N |
| ATOM | 2154 | CA | LEU | A | 226 | −25.438 | −1.661 | 36.700 | 1.00 | 49.56 | C |
| ATOM | 2155 | CB | LEU | A | 226 | −24.057 | −1.396 | 36.101 | 1.00 | 53.22 | C |
| ATOM | 2156 | CG | LEU | A | 226 | −23.295 | −0.291 | 36.834 | 1.00 | 47.55 | C |
| ATOM | 2157 | CD1 | LEU | A | 226 | −21.981 | 0.020 | 36.137 | 1.00 | 51.72 | C |
| ATOM | 2158 | CD2 | LEU | A | 226 | −24.157 | 0.960 | 36.964 | 1.00 | 35.13 | C |
| ATOM | 2159 | C | LEU | A | 226 | −26.173 | −2.776 | 35.939 | 1.00 | 47.44 | C |
| ATOM | 2160 | O | LEU | A | 226 | −26.991 | −2.547 | 35.072 | 1.00 | 51.75 | O |
| ATOM | 2161 | N | ARG | A | 227 | −25.868 | −3.998 | 36.295 | 1.00 | 47.02 | N |
| ATOM | 2162 | CA | ARG | A | 227 | −26.493 | −5.161 | 35.719 | 1.00 | 54.24 | C |
| ATOM | 2163 | CB | ARG | A | 227 | −25.718 | −6.427 | 36.084 | 1.00 | 54.50 | C |
| ATOM | 2164 | CG | ARG | A | 227 | −24.280 | −6.394 | 35.582 | 1.00 | 48.89 | C |
| ATOM | 2165 | CD | ARG | A | 227 | −23.529 | −7.654 | 35.944 | 1.00 | 47.70 | C |
| ATOM | 2166 | NE | ARG | A | 227 | −24.049 | −8.822 | 35.246 | 1.00 | 51.55 | N |
| ATOM | 2167 | CZ | ARG | A | 227 | −23.657 | −10.066 | 35.496 | 1.00 | 56.24 | C |
| ATOM | 2168 | NH1 | ARG | A | 227 | −22.746 | −10.297 | 36.433 | 1.00 | 42.21 | N |
| ATOM | 2169 | NH2 | ARG | A | 227 | −24.181 | −11.077 | 34.815 | 1.00 | 64.76 | N |
| ATOM | 2170 | C | ARG | A | 227 | −27.976 | −5.249 | 36.115 | 1.00 | 51.99 | C |
| ATOM | 2171 | O | ARG | A | 227 | −28.834 | −5.615 | 35.332 | 1.00 | 46.24 | O |
| ATOM | 2172 | N | ALA | A | 228 | −28.265 | −4.890 | 37.358 | 1.00 | 52.46 | N |
| ATOM | 2173 | CA | ALA | A | 228 | −29.596 | −5.002 | 37.908 | 1.00 | 45.57 | C |
| ATOM | 2174 | CB | ALA | A | 228 | −29.582 | −4.644 | 39.389 | 1.00 | 48.45 | C |
| ATOM | 2175 | C | ALA | A | 228 | −30.643 | −4.171 | 37.172 | 1.00 | 46.15 | C |
| ATOM | 2176 | O | ALA | A | 228 | −31.757 | −4.608 | 36.994 | 1.00 | 51.81 | O |
| ATOM | 2177 | N | TRP | A | 229 | −30.290 | −2.994 | 36.696 | 1.00 | 45.94 | N |
| ATOM | 2178 | CA | TRP | A | 229 | −31.253 | −2.187 | 35.939 | 1.00 | 48.00 | C |
| ATOM | 2179 | CB | TRP | A | 229 | −30.763 | −0.750 | 35.669 | 1.00 | 53.88 | C |
| ATOM | 2180 | CG | TRP | A | 229 | −30.949 | 0.114 | 36.881 | 1.00 | 57.07 | C |
| ATOM | 2181 | CD1 | TRP | A | 229 | −29.984 | 0.539 | 37.742 | 1.00 | 67.09 | C |
| ATOM | 2182 | NE1 | TRP | A | 229 | −30.549 | 1.265 | 38.763 | 1.00 | 74.38 | N |
| ATOM | 2183 | CE2 | TRP | A | 229 | −31.905 | 1.310 | 38.581 | 1.00 | 66.66 | C |
| ATOM | 2184 | CD2 | TRP | A | 229 | −32.196 | 0.591 | 37.407 | 1.00 | 63.25 | C |
| ATOM | 2185 | CE3 | TRP | A | 229 | −33.529 | 0.487 | 36.993 | 1.00 | 68.42 | C |
| ATOM | 2186 | CZ3 | TRP | A | 229 | −34.512 | 1.097 | 37.758 | 1.00 | 68.85 | C |
| ATOM | 2187 | CH2 | TRP | A | 229 | −34.184 | 1.806 | 38.923 | 1.00 | 66.23 | C |
| ATOM | 2188 | CZ2 | TRP | A | 229 | −32.890 | 1.922 | 39.347 | 1.00 | 70.10 | C |
| ATOM | 2189 | C | TRP | A | 229 | −31.760 | −2.885 | 34.692 | 1.00 | 45.18 | C |
| ATOM | 2190 | O | TRP | A | 229 | −32.891 | −2.746 | 34.303 | 1.00 | 40.87 | O |
| ATOM | 2191 | N | MET | A | 230 | −30.899 | −3.598 | 34.026 | 1.00 | 56.58 | N |
| ATOM | 2192 | CA | MET | A | 230 | −31.304 | −4.401 | 32.882 | 1.00 | 56.72 | C |
| ATOM | 2193 | CB | MET | A | 230 | −30.088 | −5.001 | 32.165 | 1.00 | 61.56 | C |
| ATOM | 2194 | CG | MET | A | 230 | −29.040 | −3.985 | 31.677 | 1.00 | 67.07 | C |
| ATOM | 2195 | SD | MET | A | 230 | −29.587 | −2.787 | 30.432 | 1.00 | 91.79 | S |
| ATOM | 2196 | CE | MET | A | 230 | −30.059 | −1.393 | 31.456 | 1.00 | 68.67 | C |
| ATOM | 2197 | C | MET | A | 230 | −32.340 | −5.513 | 33.238 | 1.00 | 54.08 | C |
| ATOM | 2198 | O | MET | A | 230 | −33.330 | −5.678 | 32.557 | 1.00 | 54.26 | O |
| ATOM | 2199 | N | PHE | A | 231 | −32.149 | −6.223 | 34.358 | 1.00 | 51.93 | N |
| ATOM | 2200 | CA | PHE | A | 231 | −33.173 | −7.159 | 34.860 | 1.00 | 47.96 | C |
| ATOM | 2201 | CB | PHE | A | 231 | −32.637 | −7.987 | 36.030 | 1.00 | 38.82 | C |
| ATOM | 2202 | CG | PHE | A | 231 | −31.648 | −9.039 | 35.617 | 1.00 | 44.79 | C |
| ATOM | 2203 | CD1 | PHE | A | 231 | −32.082 | −10.272 | 35.156 | 1.00 | 47.31 | C |
| ATOM | 2204 | CE1 | PHE | A | 231 | −31.173 | −11.249 | 34.770 | 1.00 | 48.96 | C |
| ATOM | 2205 | CZ | PHE | A | 231 | −29.818 | −10.996 | 34.839 | 1.00 | 48.81 | C |
| ATOM | 2206 | CE2 | PHE | A | 231 | −29.372 | −9.767 | 35.294 | 1.00 | 48.87 | C |
| ATOM | 2207 | CD2 | PHE | A | 231 | −30.285 | −8.795 | 35.679 | 1.00 | 47.30 | C |
| ATOM | 2208 | C | PHE | A | 231 | −34.517 | −6.496 | 35.215 | 1.00 | 47.95 | C |
| ATOM | 2209 | O | PHE | A | 231 | −35.557 | −6.926 | 34.771 | 1.00 | 46.60 | O |
| ATOM | 2210 | N | ILE | A | 232 | −34.451 | −5.381 | 35.955 | 1.00 | 47.12 | N |
| ATOM | 2211 | CA | ILE | A | 232 | −35.598 | −4.509 | 36.287 | 1.00 | 41.56 | C |
| ATOM | 2212 | CB | ILE | A | 232 | −35.162 | −3.212 | 36.992 | 1.00 | 41.25 | C |
| ATOM | 2213 | CG1 | ILE | A | 232 | −34.660 | −3.547 | 38.396 | 1.00 | 44.21 | C |
| ATOM | 2214 | CD1 | ILE | A | 232 | −34.324 | −2.338 | 39.229 | 1.00 | 57.07 | C |
| ATOM | 2215 | CG2 | ILE | A | 232 | −36.311 | −2.222 | 37.084 | 1.00 | 35.53 | C |
| ATOM | 2216 | C | ILE | A | 232 | −36.555 | −4.230 | 35.128 | 1.00 | 41.60 | C |
| ATOM | 2217 | O | ILE | A | 232 | −37.748 | −4.285 | 35.275 | 1.00 | 41.18 | O |
| ATOM | 2218 | N | CYS | A | 233 | −36.024 | −3.914 | 33.958 | 1.00 | 48.55 | N |
| ATOM | 2219 | CA | CYS | A | 233 | −36.855 | −3.683 | 32.799 | 1.00 | 47.31 | C |
| ATOM | 2220 | CB | CYS | A | 233 | −36.021 | −3.185 | 31.613 | 1.00 | 49.67 | C |
| ATOM | 2221 | SG | CYS | A | 233 | −36.030 | −1.395 | 31.361 | 1.00 | 60.42 | S |
| ATOM | 2222 | C | CYS | A | 233 | −37.603 | −4.936 | 32.389 | 1.00 | 45.18 | C |
| ATOM | 2223 | O | CYS | A | 233 | −38.785 | −4.901 | 32.171 | 1.00 | 53.63 | O |
| ATOM | 2224 | N | ILE | A | 234 | −36.928 | −6.066 | 32.358 | 1.00 | 43.82 | N |
| ATOM | 2225 | CA | ILE | A | 234 | −37.585 | −7.318 | 32.036 | 1.00 | 45.09 | C |
| ATOM | 2226 | CB | ILE | A | 234 | −36.594 | −8.485 | 32.056 | 1.00 | 46.98 | C |
| ATOM | 2227 | CG1 | ILE | A | 234 | −35.516 | −8.264 | 30.996 | 1.00 | 46.29 | C |
| ATOM | 2228 | CD1 | ILE | A | 234 | −34.472 | −9.353 | 30.962 | 1.00 | 56.35 | C |
| ATOM | 2229 | CG2 | ILE | A | 234 | −37.311 | −9.797 | 31.815 | 1.00 | 40.32 | C |
| ATOM | 2230 | C | ILE | A | 234 | −38.751 | −7.620 | 32.966 | 1.00 | 45.55 | C |
| ATOM | 2231 | O | ILE | A | 234 | −39.808 | −7.986 | 32.527 | 1.00 | 48.41 | O |
| ATOM | 2232 | N | GLY | A | 235 | −38.566 | −7.398 | 34.251 | 1.00 | 43.08 | N |
| ATOM | 2233 | CA | GLY | A | 235 | −39.575 | −7.699 | 35.228 | 1.00 | 43.90 | C |
| ATOM | 2234 | C | GLY | A | 235 | −40.678 | −6.716 | 35.518 | 1.00 | 40.25 | C |
| ATOM | 2235 | O | GLY | A | 235 | −41.747 | −7.149 | 35.874 | 1.00 | 38.60 | O |
| ATOM | 2236 | N | TRP | A | 236 | −40.463 | −5.424 | 35.405 | 1.00 | 36.76 | N |
| ATOM | 2237 | CA | TRP | A | 236 | −41.497 | −4.452 | 35.707 | 1.00 | 42.32 | C |
| ATOM | 2238 | CB | TRP | A | 236 | −41.067 | −3.520 | 36.846 | 1.00 | 43.95 | C |
| ATOM | 2239 | CG | TRP | A | 236 | −40.726 | −4.185 | 38.141 | 1.00 | 43.11 | C |
| ATOM | 2240 | CD1 | TRP | A | 236 | −39.478 | −4.417 | 38.634 | 1.00 | 42.79 | C |
| ATOM | 2241 | NE1 | TRP | A | 236 | −39.557 | −5.038 | 39.853 | 1.00 | 48.74 | N |
| ATOM | 2242 | CE2 | TRP | A | 236 | −40.876 | −5.215 | 40.176 | 1.00 | 51.40 | C |
| ATOM | 2243 | CD2 | TRP | A | 236 | −41.645 | −4.684 | 39.121 | 1.00 | 51.02 | C |
| ATOM | 2244 | CE3 | TRP | A | 236 | −43.040 | −4.739 | 39.209 | 1.00 | 52.15 | C |
| ATOM | 2245 | CZ3 | TRP | A | 236 | −43.611 | −5.315 | 40.331 | 1.00 | 50.68 | C |
| ATOM | 2246 | CH2 | TRP | A | 236 | −42.816 | −5.836 | 41.362 | 1.00 | 50.55 | C |
| ATOM | 2247 | CZ2 | TRP | A | 236 | −41.452 | −5.795 | 41.302 | 1.00 | 47.40 | C |
| ATOM | 2248 | C | TRP | A | 236 | −41.761 | −3.588 | 34.475 | 1.00 | 47.78 | C |
| ATOM | 2249 | O | TRP | A | 236 | −42.878 | −3.232 | 34.147 | 1.00 | 46.13 | O |
| ATOM | 2250 | N | GLY | A | 237 | −40.693 | −3.285 | 33.760 | 1.00 | 51.75 | N |
| ATOM | 2251 | CA | GLY | A | 237 | −40.783 | −2.485 | 32.583 | 1.00 | 52.25 | C |
| ATOM | 2252 | C | GLY | A | 237 | −41.526 | −3.033 | 31.414 | 1.00 | 50.30 | C |
| ATOM | 2253 | O | GLY | A | 237 | −42.450 | −2.410 | 30.928 | 1.00 | 52.50 | O |
| ATOM | 2254 | N | VAL | A | 238 | −41.145 | −4.220 | 30.957 | 1.00 | 47.08 | N |
| ATOM | 2255 | CA | VAL | A | 238 | −41.781 | −4.833 | 29.817 | 1.00 | 45.13 | C |
| ATOM | 2256 | CB | VAL | A | 238 | −41.006 | −6.074 | 29.320 | 1.00 | 38.63 | C |
| ATOM | 2257 | CG1 | VAL | A | 238 | −41.622 | −6.611 | 28.038 | 1.00 | 30.18 | C |
| ATOM | 2258 | CG2 | VAL | A | 238 | −39.552 | −5.720 | 29.093 | 1.00 | 41.33 | C |
| ATOM | 2259 | C | VAL | A | 238 | −43.222 | −5.184 | 30.111 | 1.00 | 48.09 | C |
| ATOM | 2260 | O | VAL | A | 238 | −44.046 | −4.981 | 29.250 | 1.00 | 53.82 | O |
| ATOM | 2261 | N | PRO | A | 239 | −43.661 | −5.740 | 31.229 | 1.00 | 44.99 | N |
| ATOM | 2262 | CA | PRO | A | 239 | −45.038 | −6.143 | 31.331 | 1.00 | 48.17 | C |
| ATOM | 2263 | CB | PRO | A | 239 | −45.102 | −6.900 | 32.668 | 1.00 | 49.72 | C |
| ATOM | 2264 | CG | PRO | A | 239 | −43.863 | −6.546 | 33.397 | 1.00 | 47.28 | C |
| ATOM | 2265 | CD | PRO | A | 239 | −42.857 | −6.286 | 32.333 | 1.00 | 44.15 | C |
| ATOM | 2266 | C | PRO | A | 239 | −45.962 | −4.925 | 31.370 | 1.00 | 46.87 | C |
| ATOM | 2267 | O | PRO | A | 239 | −47.098 | −5.044 | 30.975 | 1.00 | 45.62 | O |
| ATOM | 2268 | N | PHE | A | 240 | −45.479 | −3.757 | 31.768 | 1.00 | 51.30 | N |
| ATOM | 2269 | CA | PHE | A | 240 | −46.299 | −2.547 | 31.725 | 1.00 | 48.62 | C |
| ATOM | 2270 | CB | PHE | A | 240 | −45.563 | −1.361 | 32.357 | 1.00 | 50.31 | C |
| ATOM | 2271 | CG | PHE | A | 240 | −46.441 | −0.170 | 32.615 | 1.00 | 46.76 | C |
| ATOM | 2272 | CD1 | PHE | A | 240 | −47.449 | −0.228 | 33.561 | 1.00 | 51.22 | C |
| ATOM | 2273 | CE1 | PHE | A | 240 | −48.258 | 0.868 | 33.802 | 1.00 | 49.49 | C |
| ATOM | 2274 | CZ | PHE | A | 240 | −48.059 | 2.035 | 33.100 | 1.00 | 47.63 | C |
| ATOM | 2275 | CE2 | PHE | A | 240 | −47.055 | 2.107 | 32.160 | 1.00 | 46.88 | C |
| ATOM | 2276 | CD2 | PHE | A | 240 | −46.252 | 1.010 | 31.921 | 1.00 | 47.24 | C |
| ATOM | 2277 | C | PHE | A | 240 | −46.795 | −2.217 | 30.299 | 1.00 | 48.71 | C |
| ATOM | 2278 | O | PHE | A | 240 | −47.983 | −2.090 | 30.148 | 1.00 | 46.28 | O |
| ATOM | 2279 | N | PRO | A | 241 | −46.092 | −2.047 | 29.168 | 1.00 | 54.48 | N |
| ATOM | 2280 | CA | PRO | A | 241 | −46.749 | −1.751 | 27.913 | 1.00 | 50.16 | C |
| ATOM | 2281 | CB | PRO | A | 241 | −45.594 | −1.733 | 26.910 | 1.00 | 42.05 | C |
| ATOM | 2282 | CG | PRO | A | 241 | −44.439 | −1.290 | 27.706 | 1.00 | 40.13 | C |
| ATOM | 2283 | CD | PRO | A | 241 | −44.632 | −1.948 | 29.040 | 1.00 | 44.89 | C |
| ATOM | 2284 | C | PRO | A | 241 | −47.750 | −2.835 | 27.561 | 1.00 | 48.79 | C |
| ATOM | 2285 | O | PRO | A | 241 | −48.754 | −2.545 | 26.954 | 1.00 | 48.33 | O |
| ATOM | 2286 | N | ILE | A | 242 | −47.431 | −4.073 | 27.910 | 1.00 | 50.51 | N |
| ATOM | 2287 | CA | ILE | A | 242 | −48.283 | −5.213 | 27.634 | 1.00 | 50.15 | C |
| ATOM | 2288 | CB | ILE | A | 242 | −47.649 | −6.531 | 28.115 | 1.00 | 48.44 | C |
| ATOM | 2289 | CG1 | ILE | A | 242 | −46.344 | −6.785 | 27.363 | 1.00 | 34.29 | C |
| ATOM | 2290 | CD1 | ILE | A | 242 | −45.718 | −8.107 | 27.687 | 1.00 | 39.54 | C |
| ATOM | 2291 | CG2 | ILE | A | 242 | −48.607 | −7.694 | 27.911 | 1.00 | 42.56 | C |
| ATOM | 2292 | C | ILE | A | 242 | −49.641 | −5.042 | 28.253 | 1.00 | 41.26 | C |
| ATOM | 2293 | O | ILE | A | 242 | −50.638 | −5.085 | 27.585 | 1.00 | 43.90 | O |
| ATOM | 2294 | N | ILE | A | 243 | −49.673 | −4.791 | 29.540 | 1.00 | 39.23 | N |
| ATOM | 2295 | CA | ILE | A | 243 | −50.936 | −4.576 | 30.212 | 1.00 | 47.52 | C |
| ATOM | 2296 | CB | ILE | A | 243 | −50.760 | −4.522 | 31.741 | 1.00 | 45.07 | C |
| ATOM | 2297 | CG1 | ILE | A | 243 | −50.337 | −5.897 | 32.242 | 1.00 | 53.60 | C |
| ATOM | 2298 | CD1 | ILE | A | 243 | −51.275 | −7.011 | 31.828 | 1.00 | 54.72 | C |
| ATOM | 2299 | CG2 | ILE | A | 243 | −52.045 | −4.098 | 32.437 | 1.00 | 34.63 | C |
| ATOM | 2300 | C | ILE | A | 243 | −51.683 | −3.345 | 29.712 | 1.00 | 47.90 | C |
| ATOM | 2301 | O | ILE | A | 243 | −52.872 | −3.366 | 29.576 | 1.00 | 48.36 | O |
| ATOM | 2302 | N | VAL | A | 244 | −50.979 | −2.283 | 29.362 | 1.00 | 48.44 | N |
| ATOM | 2303 | CA | VAL | A | 244 | −51.616 | −1.114 | 28.779 | 1.00 | 43.98 | C |
| ATOM | 2304 | CB | VAL | A | 244 | −50.614 | 0.043 | 28.557 | 1.00 | 38.71 | C |
| ATOM | 2305 | CG1 | VAL | A | 244 | −51.252 | 1.154 | 27.743 | 1.00 | 42.32 | C |
| ATOM | 2306 | CG2 | VAL | A | 244 | −50.125 | 0.582 | 29.888 | 1.00 | 39.06 | C |
| ATOM | 2307 | C | VAL | A | 244 | −52.322 | −1.455 | 27.470 | 1.00 | 48.87 | C |
| ATOM | 2308 | O | VAL | A | 244 | −53.456 | −1.099 | 27.271 | 1.00 | 49.09 | O |
| ATOM | 2309 | N | ALA | A | 245 | −51.669 | −2.205 | 26.598 | 1.00 | 48.82 | N |
| ATOM | 2310 | CA | ALA | A | 245 | −52.291 | −2.646 | 25.366 | 1.00 | 49.56 | C |
| ATOM | 2311 | CB | ALA | A | 245 | −51.273 | −3.368 | 24.492 | 1.00 | 50.03 | C |
| ATOM | 2312 | C | ALA | A | 245 | −53.512 | −3.536 | 25.613 | 1.00 | 53.99 | C |
| ATOM | 2313 | O | ALA | A | 245 | −54.535 | −3.402 | 24.982 | 1.00 | 54.24 | O |
| ATOM | 2314 | N | TRP | A | 246 | −53.434 | −4.412 | 26.590 | 1.00 | 49.88 | N |
| ATOM | 2315 | CA | TRP | A | 246 | −54.581 | −5.203 | 26.978 | 1.00 | 50.36 | C |
| ATOM | 2316 | CB | TRP | A | 246 | −54.197 | −6.142 | 28.119 | 1.00 | 50.55 | C |
| ATOM | 2317 | CG | TRP | A | 246 | −55.332 | −6.917 | 28.723 | 1.00 | 51.42 | C |
| ATOM | 2318 | CD1 | TRP | A | 246 | −55.879 | −8.074 | 28.249 | 1.00 | 57.09 | C |
| ATOM | 2319 | NE1 | TRP | A | 246 | −56.881 | −8.503 | 29.087 | 1.00 | 55.16 | N |
| ATOM | 2320 | CE2 | TRP | A | 246 | −56.990 | −7.625 | 30.133 | 1.00 | 49.37 | C |
| ATOM | 2321 | CD2 | TRP | A | 246 | −56.027 | −6.614 | 29.939 | 1.00 | 49.33 | C |
| ATOM | 2322 | CE3 | TRP | A | 246 | −55.933 | −5.584 | 30.881 | 1.00 | 53.12 | C |
| ATOM | 2323 | CZ3 | TRP | A | 246 | −56.792 | −5.596 | 31.969 | 1.00 | 49.12 | C |
| ATOM | 2324 | CH2 | TRP | A | 246 | −57.738 | −6.617 | 32.131 | 1.00 | 49.32 | C |
| ATOM | 2325 | CZ2 | TRP | A | 246 | −57.850 | −7.637 | 31.227 | 1.00 | 49.10 | C |
| ATOM | 2326 | C | TRP | A | 246 | −55.783 | −4.352 | 27.394 | 1.00 | 49.87 | C |
| ATOM | 2327 | O | TRP | A | 246 | −56.901 | −4.643 | 27.040 | 1.00 | 53.94 | O |
| ATOM | 2328 | N | ALA | A | 247 | −55.533 | −3.335 | 28.198 | 1.00 | 48.80 | N |
| ATOM | 2329 | CA | ALA | A | 247 | −56.577 | −2.506 | 28.775 | 1.00 | 49.50 | C |
| ATOM | 2330 | CB | ALA | A | 247 | −55.995 | −1.575 | 29.831 | 1.00 | 42.45 | C |
| ATOM | 2331 | C | ALA | A | 247 | −57.306 | −1.718 | 27.717 | 1.00 | 57.78 | C |
| ATOM | 2332 | O | ALA | A | 247 | −58.504 | −1.572 | 27.770 | 1.00 | 65.02 | O |
| ATOM | 2333 | N | ILE | A | 248 | −56.575 | −1.306 | 26.687 | 1.00 | 55.60 | N |
| ATOM | 2334 | CA | ILE | A | 248 | −57.172 | −0.678 | 25.525 | 1.00 | 52.08 | C |
| ATOM | 2335 | CB | ILE | A | 248 | −56.096 | −0.170 | 24.562 | 1.00 | 56.27 | C |
| ATOM | 2336 | CG1 | ILE | A | 248 | −55.317 | 0.964 | 25.227 | 1.00 | 56.76 | C |
| ATOM | 2337 | CD1 | ILE | A | 248 | −54.270 | 1.582 | 24.342 | 1.00 | 62.74 | C |
| ATOM | 2338 | CG2 | ILE | A | 248 | −56.718 | 0.301 | 23.260 | 1.00 | 66.02 | C |
| ATOM | 2339 | C | ILE | A | 248 | −58.113 | −1.633 | 24.818 | 1.00 | 49.59 | C |
| ATOM | 2340 | O | ILE | A | 248 | −59.202 | −1.273 | 24.449 | 1.00 | 55.27 | O |
| ATOM | 2341 | N | GLY | A | 249 | −57.689 | −2.872 | 24.663 | 1.00 | 50.31 | N |
| ATOM | 2342 | CA | GLY | A | 249 | −58.485 | −3.908 | 24.058 | 1.00 | 57.87 | C |
| ATOM | 2343 | C | GLY | A | 249 | −59.798 | −4.161 | 24.708 | 1.00 | 57.78 | C |
| ATOM | 2344 | O | GLY | A | 249 | −60.830 | −4.282 | 24.078 | 1.00 | 54.55 | O |
| ATOM | 2345 | N | LYS | A | 250 | −59.773 | −4.212 | 26.012 | 1.00 | 55.50 | N |
| ATOM | 2346 | CA | LYS | A | 250 | −60.976 | −4.315 | 26.771 | 1.00 | 56.59 | C |
| ATOM | 2347 | CB | LYS | A | 250 | −60.637 | −4.529 | 28.250 | 1.00 | 56.36 | C |
| ATOM | 2348 | CG | LYS | A | 250 | −60.026 | −5.880 | 28.572 | 1.00 | 49.70 | C |
| ATOM | 2349 | CD | LYS | A | 250 | −61.083 | −6.971 | 28.558 | 1.00 | 57.23 | C |
| ATOM | 2350 | CE | LYS | A | 250 | −60.520 | −8.294 | 29.053 | 1.00 | 62.68 | C |
| ATOM | 2351 | NZ | LYS | A | 250 | −61.549 | −9.368 | 29.108 | 1.00 | 61.05 | N |
| ATOM | 2352 | C | LYS | A | 250 | −61.856 | −3.068 | 26.623 | 1.00 | 59.38 | C |
| ATOM | 2353 | O | LYS | A | 250 | −63.058 | −3.155 | 26.472 | 1.00 | 65.08 | O |
| ATOM | 2354 | N | LEU | A | 251 | −61.264 | −1.899 | 26.634 | 1.00 | 56.36 | N |
| ATOM | 2355 | CA | LEU | A | 251 | −62.072 | −0.721 | 26.486 | 1.00 | 55.62 | C |
| ATOM | 2356 | CB | LEU | A | 251 | −61.175 | 0.511 | 26.618 | 1.00 | 45.09 | C |
| ATOM | 2357 | CG | LEU | A | 251 | −61.791 | 1.902 | 26.679 | 1.00 | 55.62 | C |
| ATOM | 2358 | CD2 | LEU | A | 251 | −60.791 | 2.868 | 27.286 | 1.00 | 44.23 | C |
| ATOM | 2359 | CD1 | LEU | A | 251 | −63.066 | 1.886 | 27.494 | 1.00 | 65.98 | C |
| ATOM | 2360 | C | LEU | A | 251 | −62.753 | −0.767 | 25.152 | 1.00 | 62.24 | C |
| ATOM | 2361 | O | LEU | A | 251 | −63.876 | −0.347 | 25.021 | 1.00 | 67.60 | O |
| ATOM | 2362 | N | TYR | A | 252 | −62.090 | −1.217 | 24.133 | 1.00 | 60.52 | N |
| ATOM | 2363 | CA | TYR | A | 252 | −62.644 | −1.268 | 22.796 | 1.00 | 57.94 | C |
| ATOM | 2364 | CB | TYR | A | 252 | −61.518 | −1.399 | 21.767 | 1.00 | 65.22 | C |
| ATOM | 2365 | CG | TYR | A | 252 | −60.942 | −0.080 | 21.312 | 1.00 | 82.70 | C |
| ATOM | 2366 | CD1 | TYR | A | 252 | −61.754 | 1.036 | 21.142 | 1.00 | 81.35 | C |
| ATOM | 2367 | CE1 | TYR | A | 252 | −61.228 | 2.244 | 20.725 | 1.00 | 84.06 | C |
| ATOM | 2368 | CZ | TYR | A | 252 | −59.878 | 2.347 | 20.473 | 1.00 | 91.94 | C |
| ATOM | 2369 | OH | TYR | A | 252 | −59.356 | 3.549 | 20.057 | 1.00 | 104.53 | O |
| ATOM | 2370 | CE2 | TYR | A | 252 | −59.050 | 1.253 | 20.633 | 1.00 | 92.15 | C |
| ATOM | 2371 | CD2 | TYR | A | 252 | −59.584 | 0.051 | 21.050 | 1.00 | 88.42 | C |
| ATOM | 2372 | C | TYR | A | 252 | −63.700 | −2.293 | 22.486 | 1.00 | 64.07 | C |
| ATOM | 2373 | O | TYR | A | 252 | −64.713 | −1.952 | 21.911 | 1.00 | 75.48 | O |
| ATOM | 2374 | N | TYR | A | 253 | −63.437 | −3.519 | 22.855 | 1.00 | 63.62 | N |
| ATOM | 2375 | CA | TYR | A | 253 | −64.448 | −4.540 | 22.553 | 1.00 | 68.18 | C |
| ATOM | 2376 | CB | TYR | A | 253 | −64.024 | −5.477 | 21.405 | 1.00 | 67.22 | C |
| ATOM | 2377 | CG | TYR | A | 253 | −62.658 | −5.243 | 20.795 | 1.00 | 67.92 | C |
| ATOM | 2378 | CD1 | TYR | A | 253 | −62.446 | −4.220 | 19.876 | 1.00 | 69.90 | C |
| ATOM | 2379 | CE1 | TYR | A | 253 | −61.198 | −4.018 | 19.304 | 1.00 | 68.63 | C |
| ATOM | 2380 | CZ | TYR | A | 253 | −60.152 | −4.854 | 19.633 | 1.00 | 57.89 | C |
| ATOM | 2381 | OH | TYR | A | 253 | −58.912 | −4.659 | 19.066 | 1.00 | 51.85 | O |
| ATOM | 2382 | CE2 | TYR | A | 253 | −60.344 | −5.887 | 20.529 | 1.00 | 61.99 | C |
| ATOM | 2383 | CD2 | TYR | A | 253 | −61.592 | −6.080 | 21.097 | 1.00 | 61.77 | C |
| ATOM | 2384 | C | TYR | A | 253 | −64.985 | −5.362 | 23.697 | 1.00 | 63.88 | C |
| ATOM | 2385 | O | TYR | A | 253 | −65.848 | −6.176 | 23.505 | 1.00 | 74.42 | O |
| ATOM | 2386 | N | ASP | A | 254 | −64.479 | −5.168 | 24.882 | 1.00 | 56.26 | N |
| ATOM | 2387 | CA | ASP | A | 254 | −64.931 | −5.952 | 26.009 | 1.00 | 56.13 | C |
| ATOM | 2388 | CB | ASP | A | 254 | −63.908 | −7.083 | 26.231 | 1.00 | 64.29 | C |
| ATOM | 2389 | CG | ASP | A | 254 | −64.456 | −8.219 | 27.078 | 1.00 | 79.51 | C |
| ATOM | 2390 | OD2 | ASP | A | 254 | −63.654 | −9.045 | 27.567 | 1.00 | 84.95 | O |
| ATOM | 2391 | OD1 | ASP | A | 254 | −65.691 | −8.280 | 27.255 | 1.00 | 82.33 | O |
| ATOM | 2392 | C | ASP | A | 254 | −65.247 | −5.294 | 27.350 | 1.00 | 59.56 | C |
| ATOM | 2393 | O | ASP | A | 254 | −64.822 | −5.755 | 28.386 | 1.00 | 61.77 | O |
| ATOM | 2394 | N | ASN | A | 255 | −65.849 | −4.116 | 27.304 | 1.00 | 63.33 | N |
| ATOM | 2395 | CA | ASN | A | 255 | −65.997 | −3.226 | 28.450 | 1.00 | 57.49 | C |
| ATOM | 2396 | CB | ASN | A | 255 | −66.573 | −1.888 | 27.994 | 1.00 | 53.99 | C |
| ATOM | 2397 | CG | ASN | A | 255 | −65.906 | −0.712 | 28.667 | 1.00 | 51.84 | C |
| ATOM | 2398 | OD1 | ASN | A | 255 | −65.340 | −0.839 | 29.754 | 1.00 | 51.50 | O |
| ATOM | 2399 | ND2 | ASN | A | 255 | −65.971 | 0.446 | 28.024 | 1.00 | 63.12 | N |
| ATOM | 2400 | C | ASN | A | 255 | −66.846 | −3.788 | 29.606 | 1.00 | 57.48 | C |
| ATOM | 2401 | O | ASN | A | 255 | −67.009 | −3.152 | 30.625 | 1.00 | 56.33 | O |
| ATOM | 2402 | N | GLU | A | 256 | −67.375 | −4.978 | 29.447 | 1.00 | 59.99 | N |
| ATOM | 2403 | CA | GLU | A | 256 | −68.169 | −5.643 | 30.474 | 1.00 | 68.49 | C |
| ATOM | 2404 | CB | GLU | A | 256 | −69.085 | −6.738 | 29.886 | 1.00 | 69.07 | C |
| ATOM | 2405 | CG | GLU | A | 256 | −68.381 | −7.999 | 29.380 | 1.00 | 73.50 | C |
| ATOM | 2406 | CD | GLU | A | 256 | −69.115 | −8.614 | 28.212 | 1.00 | 94.88 | C |
| ATOM | 2407 | OE1 | GLU | A | 256 | −70.365 | −8.545 | 28.230 | 1.00 | 96.68 | O |
| ATOM | 2408 | OE2 | GLU | A | 256 | −68.455 | −9.137 | 27.274 | 1.00 | 104.73 | O |
| ATOM | 2409 | C | GLU | A | 256 | −67.373 | −6.182 | 31.698 | 1.00 | 66.20 | C |
| ATOM | 2410 | O | GLU | A | 256 | −66.311 | −6.754 | 31.558 | 1.00 | 66.92 | O |
| ATOM | 2411 | N | LYS | A | 257 | −68.127 | −6.331 | 32.784 | 1.00 | 61.95 | N |
| ATOM | 2412 | CA | LYS | A | 257 | −67.796 | −7.244 | 33.859 | 1.00 | 60.22 | C |
| ATOM | 2413 | CB | LYS | A | 257 | −67.977 | −8.702 | 33.404 | 1.00 | 64.00 | C |
| ATOM | 2414 | CG | LYS | A | 257 | −69.428 | −9.102 | 33.138 | 1.00 | 75.22 | C |
| ATOM | 2415 | CD | LYS | A | 257 | −69.541 | −10.495 | 32.507 | 1.00 | 85.40 | C |
| ATOM | 2416 | CE | LYS | A | 257 | −69.111 | −11.604 | 33.467 | 1.00 | 92.08 | C |
| ATOM | 2417 | NZ | LYS | A | 257 | −69.182 | −12.977 | 32.874 | 1.00 | 77.14 | N |
| ATOM | 2418 | C | LYS | A | 257 | −66.443 | −7.070 | 34.506 | 1.00 | 61.40 | C |
| ATOM | 2419 | O | LYS | A | 257 | −65.783 | −8.042 | 34.849 | 1.00 | 63.71 | O |
| ATOM | 2420 | N | CYS | A | 258 | −66.026 | −5.823 | 34.685 | 1.00 | 65.22 | N |
| ATOM | 2421 | CA | CYS | A | 258 | −64.753 | −5.522 | 35.328 | 1.00 | 61.22 | C |
| ATOM | 2422 | CB | CYS | A | 258 | −64.756 | −5.964 | 36.793 | 1.00 | 57.39 | C |
| ATOM | 2423 | SG | CYS | A | 258 | −66.094 | −5.239 | 37.752 | 1.00 | 61.76 | S |
| ATOM | 2424 | C | CYS | A | 258 | −63.577 | −6.090 | 34.596 | 1.00 | 57.32 | C |
| ATOM | 2425 | O | CYS | A | 258 | −62.524 | −6.270 | 35.160 | 1.00 | 50.62 | O |
| ATOM | 2426 | N | TRP | A | 259 | −63.759 | −6.343 | 33.316 | 1.00 | 64.33 | N |
| ATOM | 2427 | CA | TRP | A | 259 | −62.688 | −6.849 | 32.523 | 1.00 | 60.65 | C |
| ATOM | 2428 | CB | TRP | A | 259 | −61.482 | −5.892 | 32.469 | 1.00 | 51.36 | C |
| ATOM | 2429 | CG | TRP | A | 259 | −61.792 | −4.575 | 31.805 | 1.00 | 56.74 | C |
| ATOM | 2430 | CD1 | TRP | A | 259 | −62.904 | −4.261 | 31.077 | 1.00 | 56.39 | C |
| ATOM | 2431 | NE1 | TRP | A | 259 | −62.829 | −2.963 | 30.632 | 1.00 | 49.96 | N |
| ATOM | 2432 | CE2 | TRP | A | 259 | −61.655 | −2.412 | 31.071 | 1.00 | 57.69 | C |
| ATOM | 2433 | CD2 | TRP | A | 259 | −60.976 | −3.399 | 31.816 | 1.00 | 59.17 | C |
| ATOM | 2434 | CE3 | TRP | A | 259 | −59.738 | −3.082 | 32.382 | 1.00 | 51.57 | C |
| ATOM | 2435 | CZ3 | TRP | A | 259 | −59.226 | −1.815 | 32.190 | 1.00 | 50.53 | C |
| ATOM | 2436 | CH2 | TRP | A | 259 | −59.926 | −0.857 | 31.447 | 1.00 | 55.36 | C |
| ATOM | 2437 | CZ2 | TRP | A | 259 | −61.139 | −1.138 | 30.881 | 1.00 | 59.25 | C |
| ATOM | 2438 | C | TRP | A | 259 | −62.303 | −8.288 | 32.848 | 1.00 | 60.57 | C |
| ATOM | 2439 | O | TRP | A | 259 | −61.230 | −8.750 | 32.506 | 1.00 | 62.33 | O |
| ATOM | 2440 | N | ALA | A | 260 | −63.162 | −8.984 | 33.584 | 1.00 | 56.12 | N |
| ATOM | 2441 | CA | ALA | A | 260 | −62.822 | −10.314 | 34.058 | 1.00 | 61.83 | C |
| ATOM | 2442 | CB | ALA | A | 260 | −62.773 | −10.330 | 35.577 | 1.00 | 61.69 | C |
| ATOM | 2443 | C | ALA | A | 260 | −63.798 | −11.388 | 33.548 | 1.00 | 66.72 | C |
| ATOM | 2444 | O | ALA | A | 260 | −63.991 | −12.410 | 34.194 | 1.00 | 75.79 | O |
| ATOM | 2445 | N | GLY | A | 261 | −64.613 | −10.929 | 32.602 | 1.00 | 72.38 | N |
| ATOM | 2446 | CA | GLY | A | 261 | −65.697 | −11.694 | 32.032 | 1.00 | 83.00 | C |
| ATOM | 2447 | C | GLY | A | 261 | −65.022 | −12.636 | 31.089 | 1.00 | 86.85 | C |
| ATOM | 2448 | O | GLY | A | 261 | −63.950 | −12.318 | 30.575 | 1.00 | 78.92 | O |
| ATOM | 2449 | N | LYS | A | 262 | −65.602 | −13.798 | 30.850 | 1.00 | 94.36 | N |
| ATOM | 2450 | CA | LYS | A | 262 | −64.819 | −14.736 | 29.991 | 1.00 | 97.29 | C |
| ATOM | 2451 | C | LYS | A | 262 | −66.001 | −14.755 | 28.960 | 1.00 | 96.85 | C |
| ATOM | 2452 | O | LYS | A | 262 | −67.122 | −15.177 | 29.238 | 1.00 | 88.95 | O |
| ATOM | 2453 | CB | LYS | A | 262 | −64.677 | −16.115 | 30.615 | 1.00 | 92.26 | C |
| ATOM | 2454 | CG | LYS | A | 262 | −63.631 | −16.237 | 31.706 | 1.00 | 97.45 | C |
| ATOM | 2455 | CD | LYS | A | 262 | −63.568 | −17.676 | 32.200 | 1.00 | 96.40 | C |
| ATOM | 2456 | CE | LYS | A | 262 | −62.363 | −17.913 | 33.092 | 1.00 | 88.53 | C |
| ATOM | 2457 | NZ | LYS | A | 262 | −62.284 | −19.329 | 33.549 | 1.00 | 82.31 | N |
| ATOM | 2458 | O | ARG | A | 263 | −65.198 | −14.750 | 24.740 | 1.00 | 121.64 | O |
| ATOM | 2459 | N | ARG | A | 263 | −65.675 | −14.332 | 27.743 | 1.00 | 108.17 | N |
| ATOM | 2460 | CA | ARG | A | 263 | −66.590 | −14.318 | 26.617 | 1.00 | 112.70 | C |
| ATOM | 2461 | C | ARG | A | 263 | −66.132 | −15.176 | 25.418 | 1.00 | 115.46 | C |
| ATOM | 2462 | CB | ARG | A | 263 | −66.840 | −12.870 | 26.186 | 1.00 | 108.74 | C |
| ATOM | 2463 | CG | ARG | A | 263 | −67.419 | −12.005 | 27.297 | 1.00 | 106.21 | C |
| ATOM | 2464 | CD | ARG | A | 263 | −68.754 | −12.564 | 27.754 | 1.00 | 115.73 | C |
| ATOM | 2465 | NE | ARG | A | 263 | −69.538 | −11.599 | 28.515 | 1.00 | 120.21 | N |
| ATOM | 2466 | CZ | ARG | A | 263 | −70.675 | −11.892 | 29.136 | 1.00 | 114.39 | C |
| ATOM | 2467 | NH1 | ARG | A | 263 | −71.157 | −13.128 | 29.092 | 1.00 | 110.66 | N |
| ATOM | 2468 | NH2 | ARG | A | 263 | −71.329 | −10.950 | 29.800 | 1.00 | 106.97 | N |
| ATOM | 2469 | O | PRO | A | 264 | −68.062 | −17.351 | 22.268 | 1.00 | 121.86 | O |
| ATOM | 2470 | N | PRO | A | 264 | −66.675 | −16.357 | 25.039 | 1.00 | 108.25 | N |
| ATOM | 2471 | CA | PRO | A | 264 | −66.240 | −17.051 | 23.804 | 1.00 | 109.19 | C |
| ATOM | 2472 | C | PRO | A | 264 | −67.119 | −16.606 | 22.560 | 1.00 | 114.87 | C |
| ATOM | 2473 | CB | PRO | A | 264 | −66.465 | −18.521 | 24.149 | 1.00 | 111.92 | C |
| ATOM | 2474 | CG | PRO | A | 264 | −67.619 | −18.500 | 25.111 | 1.00 | 111.10 | C |
| ATOM | 2475 | CD | PRO | A | 264 | −67.498 | −17.222 | 25.904 | 1.00 | 105.07 | C |
| ATOM | 2476 | O | GLY | A | 265 | −64.998 | −14.357 | 20.039 | 1.00 | 108.18 | O |
| ATOM | 2477 | N | GLY | A | 265 | −66.988 | −15.449 | 21.859 | 1.00 | 108.64 | N |
| ATOM | 2478 | CA | GLY | A | 265 | −66.156 | −14.312 | 22.101 | 1.00 | 109.98 | C |
| ATOM | 2479 | C | GLY | A | 265 | −65.027 | −13.942 | 21.199 | 1.00 | 108.39 | C |
| ATOM | 2480 | O | VAL | A | 266 | −62.155 | −12.293 | 23.194 | 1.00 | 89.58 | O |
| ATOM | 2481 | N | VAL | A | 266 | −64.120 | −13.095 | 21.741 | 1.00 | 104.14 | N |
| ATOM | 2482 | CA | VAL | A | 266 | −62.968 | −12.642 | 20.985 | 1.00 | 111.07 | C |
| ATOM | 2483 | C | VAL | A | 266 | −61.886 | −12.612 | 22.039 | 1.00 | 102.21 | C |
| ATOM | 2484 | CB | VAL | A | 266 | −63.156 | −11.207 | 20.443 | 1.00 | 116.13 | C |
| ATOM | 2485 | CG1 | VAL | A | 266 | −61.880 | −10.709 | 19.752 | 1.00 | 98.73 | C |
| ATOM | 2486 | CG2 | VAL | A | 266 | −64.350 | −11.134 | 19.500 | 1.00 | 103.95 | C |
| ATOM | 2487 | O | TYR | A | 267 | −58.086 | −11.573 | 21.854 | 1.00 | 88.17 | O |
| ATOM | 2488 | N | TYR | A | 267 | −60.712 | −13.125 | 21.623 | 1.00 | 96.52 | N |
| ATOM | 2489 | CA | TYR | A | 267 | −59.597 | −13.334 | 22.508 | 1.00 | 91.62 | C |
| ATOM | 2490 | C | TYR | A | 267 | −58.809 | −12.039 | 22.724 | 1.00 | 91.40 | C |
| ATOM | 2491 | CB | TYR | A | 267 | −58.709 | −14.450 | 21.949 | 1.00 | 91.34 | C |
| ATOM | 2492 | CG | TYR | A | 267 | −59.475 | −15.724 | 21.635 | 1.00 | 98.88 | C |
| ATOM | 2493 | CD2 | TYR | A | 267 | −59.542 | −16.763 | 22.558 | 1.00 | 105.39 | C |
| ATOM | 2494 | CD1 | TYR | A | 267 | −60.128 | −15.889 | 20.415 | 1.00 | 95.30 | C |
| ATOM | 2495 | CE2 | TYR | A | 267 | −60.234 | −17.926 | 22.276 | 1.00 | 111.32 | C |
| ATOM | 2496 | CE1 | TYR | A | 267 | −60.824 | −17.047 | 20.128 | 1.00 | 98.47 | C |
| ATOM | 2497 | CZ | TYR | A | 267 | −60.872 | −18.062 | 21.060 | 1.00 | 113.11 | C |
| ATOM | 2498 | OH | TYR | A | 267 | −61.562 | −19.219 | 20.776 | 1.00 | 111.01 | O |
| ATOM | 2499 | N | THR | A | 268 | −58.960 | −11.463 | 23.908 | 1.00 | 88.90 | N |
| ATOM | 2500 | CA | THR | A | 268 | −58.277 | −10.241 | 24.226 | 1.00 | 76.20 | C |
| ATOM | 2501 | CB | THR | A | 268 | −59.242 | −9.136 | 24.709 | 1.00 | 73.23 | C |
| ATOM | 2502 | OG1 | THR | A | 268 | −59.885 | −9.550 | 25.920 | 1.00 | 79.57 | O |
| ATOM | 2503 | CG2 | THR | A | 268 | −60.292 | −8.844 | 23.650 | 1.00 | 78.54 | C |
| ATOM | 2504 | C | THR | A | 268 | −57.267 | −10.520 | 25.297 | 1.00 | 73.22 | C |
| ATOM | 2505 | O | THR | A | 268 | −56.244 | −9.870 | 25.405 | 1.00 | 69.04 | O |
| ATOM | 2506 | N | ASP | A | 269 | −57.533 | −11.569 | 26.056 | 1.00 | 76.56 | N |
| ATOM | 2507 | CA | ASP | A | 269 | −56.645 | −11.977 | 27.107 | 1.00 | 71.03 | C |
| ATOM | 2508 | CB | ASP | A | 269 | −57.308 | −13.048 | 27.979 | 1.00 | 70.36 | C |
| ATOM | 2509 | CG | ASP | A | 269 | −58.361 | −12.477 | 28.908 | 1.00 | 68.50 | C |
| ATOM | 2510 | OD1 | ASP | A | 269 | −58.717 | −11.290 | 28.748 | 1.00 | 68.97 | O |
| ATOM | 2511 | OD2 | ASP | A | 269 | −58.835 | −13.218 | 29.795 | 1.00 | 64.93 | O |
| ATOM | 2512 | C | ASP | A | 269 | −55.316 | −12.504 | 26.563 | 1.00 | 69.21 | C |
| ATOM | 2513 | O | ASP | A | 269 | −54.397 | −12.764 | 27.309 | 1.00 | 72.33 | O |
| ATOM | 2514 | N | TYR | A | 270 | −55.225 | −12.672 | 25.253 | 1.00 | 66.73 | N |
| ATOM | 2515 | CA | TYR | A | 270 | −54.041 | −13.237 | 24.639 | 1.00 | 64.50 | C |
| ATOM | 2516 | CB | TYR | A | 270 | −54.388 | −13.920 | 23.312 | 1.00 | 80.42 | C |
| ATOM | 2517 | CG | TYR | A | 270 | −55.045 | −15.281 | 23.432 | 1.00 | 79.42 | C |
| ATOM | 2518 | CD2 | TYR | A | 270 | −55.047 | −16.160 | 22.354 | 1.00 | 75.16 | C |
| ATOM | 2519 | CE2 | TYR | A | 270 | −55.636 | −17.403 | 22.444 | 1.00 | 78.89 | C |
| ATOM | 2520 | CZ | TYR | A | 270 | −56.233 | −17.787 | 23.624 | 1.00 | 90.68 | C |
| ATOM | 2521 | OH | TYR | A | 270 | −56.823 | −19.027 | 23.716 | 1.00 | 89.08 | O |
| ATOM | 2522 | CE1 | TYR | A | 270 | −56.244 | −16.936 | 24.711 | 1.00 | 93.17 | C |
| ATOM | 2523 | CD1 | TYR | A | 270 | −55.649 | −15.692 | 24.613 | 1.00 | 79.47 | C |
| ATOM | 2524 | C | TYR | A | 270 | −53.000 | −12.168 | 24.396 | 1.00 | 51.66 | C |
| ATOM | 2525 | O | TYR | A | 270 | −51.854 | −12.451 | 24.119 | 1.00 | 49.17 | O |
| ATOM | 2526 | N | ILE | A | 271 | −53.397 | −10.929 | 24.623 | 1.00 | 54.36 | N |
| ATOM | 2527 | CA | ILE | A | 271 | −52.500 | −9.809 | 24.539 | 1.00 | 52.81 | C |
| ATOM | 2528 | CB | ILE | A | 271 | −53.248 | −8.465 | 24.586 | 1.00 | 49.57 | C |
| ATOM | 2529 | CG1 | ILE | A | 271 | −54.151 | −8.329 | 23.362 | 1.00 | 47.41 | C |
| ATOM | 2530 | CD1 | ILE | A | 271 | −54.824 | −6.984 | 23.239 | 1.00 | 46.02 | C |
| ATOM | 2531 | CG2 | ILE | A | 271 | −52.263 | −7.311 | 24.639 | 1.00 | 41.45 | C |
| ATOM | 2532 | C | ILE | A | 271 | −51.487 | −9.881 | 25.668 | 1.00 | 49.97 | C |
| ATOM | 2533 | O | ILE | A | 271 | −50.313 | −9.686 | 25.447 | 1.00 | 51.24 | O |
| ATOM | 2534 | N | TYR | A | 272 | −51.951 | −10.192 | 26.881 | 1.00 | 51.49 | N |
| ATOM | 2535 | CA | TYR | A | 272 | −51.043 | −10.461 | 27.981 | 1.00 | 50.31 | C |
| ATOM | 2536 | CB | TYR | A | 272 | −51.566 | −9.869 | 29.300 | 1.00 | 50.11 | C |
| ATOM | 2537 | CG | TYR | A | 272 | −52.539 | −10.743 | 30.063 | 1.00 | 59.12 | C |
| ATOM | 2538 | CD1 | TYR | A | 272 | −52.083 | −11.702 | 30.961 | 1.00 | 57.97 | C |
| ATOM | 2539 | CE1 | TYR | A | 272 | −52.964 | −12.507 | 31.662 | 1.00 | 63.13 | C |
| ATOM | 2540 | CZ | TYR | A | 272 | −54.321 | −12.353 | 31.481 | 1.00 | 59.53 | C |
| ATOM | 2541 | OH | TYR | A | 272 | −55.190 | −13.157 | 32.183 | 1.00 | 61.94 | O |
| ATOM | 2542 | CE2 | TYR | A | 272 | −54.803 | −11.404 | 30.603 | 1.00 | 55.97 | C |
| ATOM | 2543 | CD2 | TYR | A | 272 | −53.913 | −10.602 | 29.900 | 1.00 | 57.96 | C |
| ATOM | 2544 | C | TYR | A | 272 | −50.671 | −11.969 | 28.115 | 1.00 | 48.29 | C |
| ATOM | 2545 | O | TYR | A | 272 | −49.528 | −12.317 | 28.324 | 1.00 | 51.95 | O |
| ATOM | 2546 | N | GLN | A | 273 | −51.617 | −12.880 | 27.988 | 1.00 | 50.27 | N |
| ATOM | 2547 | CA | GLN | A | 273 | −51.289 | −14.303 | 27.982 | 1.00 | 53.83 | C |
| ATOM | 2548 | CB | GLN | A | 273 | −52.550 | −15.165 | 27.887 | 1.00 | 57.86 | C |
| ATOM | 2549 | CG | GLN | A | 273 | −53.364 | −15.150 | 29.173 | 1.00 | 57.51 | C |
| ATOM | 2550 | CD | GLN | A | 273 | −54.754 | −15.715 | 29.001 | 1.00 | 65.14 | C |
| ATOM | 2551 | OE1 | GLN | A | 273 | −55.143 | −16.121 | 27.905 | 1.00 | 66.16 | O |
| ATOM | 2552 | NE2 | GLN | A | 273 | −55.518 | −15.741 | 30.089 | 1.00 | 76.10 | N |
| ATOM | 2553 | C | GLN | A | 273 | −50.266 | −14.707 | 26.928 | 1.00 | 49.45 | C |
| ATOM | 2554 | O | GLN | A | 273 | −49.485 | −15.617 | 27.112 | 1.00 | 46.29 | O |
| ATOM | 2555 | N | GLY | A | 274 | −50.276 | −14.019 | 25.812 | 1.00 | 54.30 | N |
| ATOM | 2556 | CA | GLY | A | 274 | −49.347 | −14.303 | 24.749 | 1.00 | 52.13 | C |
| ATOM | 2557 | C | GLY | A | 274 | −47.887 | −14.261 | 25.119 | 1.00 | 51.61 | C |
| ATOM | 2558 | O | GLY | A | 274 | −47.246 | −15.291 | 25.072 | 1.00 | 54.71 | O |
| ATOM | 2559 | N | PRO | A | 275 | −47.321 | −13.119 | 25.525 | 1.00 | 50.42 | N |
| ATOM | 2560 | CA | PRO | A | 275 | −45.980 | −12.984 | 26.029 | 1.00 | 49.20 | C |
| ATOM | 2561 | CB | PRO | A | 275 | −45.932 | −11.527 | 26.501 | 1.00 | 39.72 | C |
| ATOM | 2562 | CG | PRO | A | 275 | −46.914 | −10.833 | 25.671 | 1.00 | 37.63 | C |
| ATOM | 2563 | CD | PRO | A | 275 | −48.009 | −11.816 | 25.436 | 1.00 | 43.76 | C |
| ATOM | 2564 | C | PRO | A | 275 | −45.685 | −13.886 | 27.217 | 1.00 | 51.53 | C |
| ATOM | 2565 | O | PRO | A | 275 | −44.593 | −14.401 | 27.279 | 1.00 | 56.86 | O |
| ATOM | 2566 | N | MET | A | 276 | −46.634 | −14.124 | 28.117 | 1.00 | 48.91 | N |
| ATOM | 2567 | CA | MET | A | 276 | −46.430 | −15.038 | 29.235 | 1.00 | 47.66 | C |
| ATOM | 2568 | CB | MET | A | 276 | −47.663 | −15.076 | 30.139 | 1.00 | 42.93 | C |
| ATOM | 2569 | CG | MET | A | 276 | −47.883 | −13.785 | 30.904 | 1.00 | 45.07 | C |
| ATOM | 2570 | SD | MET | A | 276 | −49.462 | −13.709 | 31.771 | 1.00 | 58.08 | S |
| ATOM | 2571 | CE | MET | A | 276 | −49.285 | −15.031 | 32.964 | 1.00 | 47.25 | C |
| ATOM | 2572 | C | MET | A | 276 | −46.047 | −16.441 | 28.762 | 1.00 | 52.79 | C |
| ATOM | 2573 | O | MET | A | 276 | −45.162 | −17.084 | 29.287 | 1.00 | 47.61 | O |
| ATOM | 2574 | N | ALA | A | 277 | −46.733 | −16.904 | 27.741 | 1.00 | 51.67 | N |
| ATOM | 2575 | CA | ALA | A | 277 | −46.441 | −18.178 | 27.154 | 1.00 | 45.65 | C |
| ATOM | 2576 | CB | ALA | A | 277 | −47.521 | −18.546 | 26.146 | 1.00 | 44.82 | C |
| ATOM | 2577 | C | ALA | A | 277 | −45.079 | −18.197 | 26.489 | 1.00 | 48.62 | C |
| ATOM | 2578 | O | ALA | A | 277 | −44.346 | −19.151 | 26.605 | 1.00 | 47.87 | O |
| ATOM | 2579 | N | LEU | A | 278 | −44.773 | −17.146 | 25.742 | 1.00 | 46.92 | N |
| ATOM | 2580 | CA | LEU | A | 278 | −43.522 | −17.068 | 24.999 | 1.00 | 51.78 | C |
| ATOM | 2581 | CB | LEU | A | 278 | −43.540 | −15.859 | 24.057 | 1.00 | 53.83 | C |
| ATOM | 2582 | CG | LEU | A | 278 | −42.208 | −15.524 | 23.375 | 1.00 | 49.56 | C |
| ATOM | 2583 | CD1 | LEU | A | 278 | −41.802 | −16.629 | 22.410 | 1.00 | 44.17 | C |
| ATOM | 2584 | CD2 | LEU | A | 278 | −42.274 | −14.180 | 22.666 | 1.00 | 46.95 | C |
| ATOM | 2585 | C | LEU | A | 278 | −42.305 | −16.988 | 25.886 | 1.00 | 53.89 | C |
| ATOM | 2586 | O | LEU | A | 278 | −41.288 | −17.611 | 25.636 | 1.00 | 56.13 | O |
| ATOM | 2587 | N | VAL | A | 279 | −42.435 | −16.239 | 26.960 | 1.00 | 53.71 | N |
| ATOM | 2588 | CA | VAL | A | 279 | −41.381 | −16.111 | 27.950 | 1.00 | 53.58 | C |
| ATOM | 2589 | CB | VAL | A | 279 | −41.804 | −15.097 | 29.029 | 1.00 | 50.73 | C |
| ATOM | 2590 | CG1 | VAL | A | 279 | −41.167 | −15.420 | 30.356 | 1.00 | 55.91 | C |
| ATOM | 2591 | CG2 | VAL | A | 279 | −41.473 | −13.676 | 28.578 | 1.00 | 47.15 | C |
| ATOM | 2592 | C | VAL | A | 279 | −41.023 | −17.466 | 28.585 | 1.00 | 54.53 | C |
| ATOM | 2593 | O | VAL | A | 279 | −39.877 | −17.818 | 28.784 | 1.00 | 50.63 | O |
| ATOM | 2594 | N | LEU | A | 280 | −42.058 | −18.244 | 28.855 | 1.00 | 53.97 | N |
| ATOM | 2595 | CA | LEU | A | 280 | −41.915 | −19.588 | 29.363 | 1.00 | 51.00 | C |
| ATOM | 2596 | CB | LEU | A | 280 | −43.287 | −20.198 | 29.640 | 1.00 | 42.65 | C |
| ATOM | 2597 | CG | LEU | A | 280 | −43.282 | −21.455 | 30.506 | 1.00 | 46.33 | C |
| ATOM | 2598 | CD1 | LEU | A | 280 | −42.714 | −21.163 | 31.885 | 1.00 | 43.34 | C |
| ATOM | 2599 | CD2 | LEU | A | 280 | −44.685 | −22.026 | 30.616 | 1.00 | 62.78 | C |
| ATOM | 2600 | C | LEU | A | 280 | −41.102 | −20.500 | 28.420 | 1.00 | 53.23 | C |
| ATOM | 2601 | O | LEU | A | 280 | −40.172 | −21.173 | 28.831 | 1.00 | 56.03 | O |
| ATOM | 2602 | N | LEU | A | 281 | −41.450 | −20.494 | 27.131 | 1.00 | 53.10 | N |
| ATOM | 2603 | CA | LEU | A | 281 | −40.770 | −21.324 | 26.135 | 1.00 | 51.15 | C |
| ATOM | 2604 | CB | LEU | A | 281 | −41.371 | −21.091 | 24.747 | 1.00 | 53.89 | C |
| ATOM | 2605 | CG | LEU | A | 281 | −40.691 | −21.813 | 23.580 | 1.00 | 56.41 | C |
| ATOM | 2606 | CD1 | LEU | A | 281 | −40.803 | −23.320 | 23.754 | 1.00 | 52.67 | C |
| ATOM | 2607 | CD2 | LEU | A | 281 | −41.269 | −21.372 | 22.237 | 1.00 | 50.75 | C |
| ATOM | 2608 | C | LEU | A | 281 | −39.288 | −21.025 | 26.097 | 1.00 | 52.48 | C |
| ATOM | 2609 | O | LEU | A | 281 | −38.445 | −21.906 | 26.149 | 1.00 | 48.16 | O |
| ATOM | 2610 | N | ILE | A | 282 | −38.992 | −19.738 | 26.065 | 1.00 | 49.42 | N |
| ATOM | 2611 | CA | ILE | A | 282 | −37.632 | −19.266 | 26.076 | 1.00 | 51.17 | C |
| ATOM | 2612 | CB | ILE | A | 282 | −37.555 | −17.728 | 26.056 | 1.00 | 46.93 | C |
| ATOM | 2613 | CG1 | ILE | A | 282 | −38.228 | −17.180 | 24.799 | 1.00 | 38.38 | C |
| ATOM | 2614 | CD1 | ILE | A | 282 | −38.147 | −15.679 | 24.672 | 1.00 | 33.40 | C |
| ATOM | 2615 | CG2 | ILE | A | 282 | −36.107 | −17.268 | 26.118 | 1.00 | 47.53 | C |
| ATOM | 2616 | C | ILE | A | 282 | −36.889 | −19.806 | 27.297 | 1.00 | 53.82 | C |
| ATOM | 2617 | O | ILE | A | 282 | −35.800 | −20.326 | 27.182 | 1.00 | 57.10 | O |
| ATOM | 2618 | N | ASN | A | 283 | −37.500 | −19.713 | 28.469 | 1.00 | 53.54 | N |
| ATOM | 2619 | CA | ASN | A | 283 | −36.908 | −20.262 | 29.676 | 1.00 | 48.95 | C |
| ATOM | 2620 | CB | ASN | A | 283 | −37.804 | −20.010 | 30.890 | 1.00 | 52.13 | C |
| ATOM | 2621 | CG | ASN | A | 283 | −37.138 | −19.125 | 31.933 | 1.00 | 54.26 | C |
| ATOM | 2622 | OD1 | ASN | A | 283 | −36.086 | −18.536 | 31.684 | 1.00 | 58.99 | O |
| ATOM | 2623 | ND2 | ASN | A | 283 | −37.753 | −19.026 | 33.105 | 1.00 | 52.44 | N |
| ATOM | 2624 | C | ASN | A | 283 | −36.592 | −21.754 | 29.532 | 1.00 | 51.23 | C |
| ATOM | 2625 | O | ASN | A | 283 | −35.562 | −22.205 | 29.977 | 1.00 | 51.20 | O |
| ATOM | 2626 | N | PHE | A | 284 | −37.466 | −22.508 | 28.859 | 1.00 | 53.29 | N |
| ATOM | 2627 | CA | PHE | A | 284 | −37.200 | −23.924 | 28.580 | 1.00 | 51.36 | C |
| ATOM | 2628 | CB | PHE | A | 284 | −38.423 | −24.632 | 27.989 | 1.00 | 50.51 | C |
| ATOM | 2629 | CG | PHE | A | 284 | −39.489 | −24.939 | 29.005 | 1.00 | 53.59 | C |
| ATOM | 2630 | CD2 | PHE | A | 284 | −40.742 | −24.354 | 28.914 | 1.00 | 48.73 | C |
| ATOM | 2631 | CE2 | PHE | A | 284 | −41.721 | −24.629 | 29.847 | 1.00 | 43.76 | C |
| ATOM | 2632 | CZ | PHE | A | 284 | −41.458 | −25.495 | 30.887 | 1.00 | 51.44 | C |
| ATOM | 2633 | CE1 | PHE | A | 284 | −40.213 | −26.087 | 30.992 | 1.00 | 55.94 | C |
| ATOM | 2634 | CD1 | PHE | A | 284 | −39.235 | −25.810 | 30.053 | 1.00 | 53.22 | C |
| ATOM | 2635 | C | PHE | A | 284 | −35.970 | −24.124 | 27.716 | 1.00 | 57.79 | C |
| ATOM | 2636 | O | PHE | A | 284 | −35.140 | −24.971 | 27.977 | 1.00 | 55.33 | O |
| ATOM | 2637 | N | ILE | A | 285 | −35.839 | −23.291 | 26.697 | 1.00 | 59.13 | N |
| ATOM | 2638 | CA | ILE | A | 285 | −34.669 | −23.313 | 25.838 | 1.00 | 57.03 | C |
| ATOM | 2639 | CB | ILE | A | 285 | −34.842 | −22.343 | 24.660 | 1.00 | 56.69 | C |
| ATOM | 2640 | CG1 | ILE | A | 285 | −35.999 | −22.821 | 23.777 | 1.00 | 46.25 | C |
| ATOM | 2641 | CD1 | ILE | A | 285 | −36.394 | −21.849 | 22.695 | 1.00 | 43.27 | C |
| ATOM | 2642 | CG2 | ILE | A | 285 | −33.551 | −22.223 | 23.865 | 1.00 | 54.60 | C |
| ATOM | 2643 | C | ILE | A | 285 | −33.397 | −23.011 | 26.619 | 1.00 | 55.57 | C |
| ATOM | 2644 | O | ILE | A | 285 | −32.424 | −23.733 | 26.519 | 1.00 | 64.12 | O |
| ATOM | 2645 | N | PHE | A | 286 | −33.462 | −21.992 | 27.479 | 1.00 | 50.68 | N |
| ATOM | 2646 | CA | PHE | A | 286 | −32.399 | −21.690 | 28.456 | 1.00 | 61.20 | C |
| ATOM | 2647 | CB | PHE | A | 286 | −32.810 | −20.530 | 29.370 | 1.00 | 61.00 | C |
| ATOM | 2648 | CG | PHE | A | 286 | −32.690 | −19.175 | 28.739 | 1.00 | 57.67 | C |
| ATOM | 2649 | CD1 | PHE | A | 286 | −32.045 | −19.009 | 27.530 | 1.00 | 57.47 | C |
| ATOM | 2650 | CE1 | PHE | A | 286 | −31.939 | −17.756 | 26.956 | 1.00 | 65.35 | C |
| ATOM | 2651 | CZ | PHE | A | 286 | −32.475 | −16.653 | 27.595 | 1.00 | 63.56 | C |
| ATOM | 2652 | CE2 | PHE | A | 286 | −33.115 | −16.805 | 28.805 | 1.00 | 58.55 | C |
| ATOM | 2653 | CD2 | PHE | A | 286 | −33.219 | −18.060 | 29.372 | 1.00 | 60.19 | C |
| ATOM | 2654 | C | PHE | A | 286 | −32.059 | −22.871 | 29.354 | 1.00 | 63.31 | C |
| ATOM | 2655 | O | PHE | A | 286 | −30.909 | −23.192 | 29.548 | 1.00 | 65.06 | O |
| ATOM | 2656 | N | LEU | A | 287 | −33.089 | −23.517 | 29.888 | 1.00 | 61.49 | N |
| ATOM | 2657 | CA | LEU | A | 287 | −32.924 | −24.644 | 30.784 | 1.00 | 61.39 | C |
| ATOM | 2658 | CB | LEU | A | 287 | −34.281 | −25.074 | 31.348 | 1.00 | 58.87 | C |
| ATOM | 2659 | CG | LEU | A | 287 | −34.292 | −26.239 | 32.338 | 1.00 | 54.01 | C |
| ATOM | 2660 | CD1 | LEU | A | 287 | −33.500 | −25.893 | 33.589 | 1.00 | 55.11 | C |
| ATOM | 2661 | CD2 | LEU | A | 287 | −35.721 | −26.624 | 32.691 | 1.00 | 55.26 | C |
| ATOM | 2662 | C | LEU | A | 287 | −32.271 | −25.796 | 30.061 | 1.00 | 59.70 | C |
| ATOM | 2663 | O | LEU | A | 287 | −31.403 | −26.466 | 30.570 | 1.00 | 59.66 | O |
| ATOM | 2664 | N | PHE | A | 288 | −32.735 | −26.021 | 28.848 | 1.00 | 58.47 | N |
| ATOM | 2665 | CA | PHE | A | 288 | −32.229 | −27.070 | 28.013 | 1.00 | 61.32 | C |
| ATOM | 2666 | CB | PHE | A | 288 | −33.008 | −27.156 | 26.691 | 1.00 | 71.83 | C |
| ATOM | 2667 | CG | PHE | A | 288 | −32.487 | −28.211 | 25.751 | 1.00 | 87.92 | C |
| ATOM | 2668 | CD1 | PHE | A | 288 | −32.244 | −29.503 | 26.198 | 1.00 | 85.91 | C |
| ATOM | 2669 | CE1 | PHE | A | 288 | −31.745 | −30.465 | 25.333 | 1.00 | 89.30 | C |
| ATOM | 2670 | CZ | PHE | A | 288 | −31.489 | −30.136 | 24.012 | 1.00 | 85.95 | C |
| ATOM | 2671 | CE2 | PHE | A | 288 | −31.728 | −28.855 | 23.562 | 1.00 | 89.01 | C |
| ATOM | 2672 | CD2 | PHE | A | 288 | −32.222 | −27.904 | 24.425 | 1.00 | 95.56 | C |
| ATOM | 2673 | C | PHE | A | 288 | −30.741 | −26.877 | 27.755 | 1.00 | 71.60 | C |
| ATOM | 2674 | O | PHE | A | 288 | −29.940 | −27.773 | 27.949 | 1.00 | 76.52 | O |
| ATOM | 2675 | N | ASN | A | 289 | −30.379 | −25.658 | 27.403 | 1.00 | 70.59 | N |
| ATOM | 2676 | CA | ASN | A | 289 | −28.989 | −25.294 | 27.224 | 1.00 | 67.19 | C |
| ATOM | 2677 | CB | ASN | A | 289 | −28.927 | −23.866 | 26.672 | 1.00 | 63.34 | C |
| ATOM | 2678 | CG | ASN | A | 289 | −27.599 | −23.538 | 26.018 | 1.00 | 73.99 | C |
| ATOM | 2679 | OD1 | ASN | A | 289 | −26.545 | −24.012 | 26.440 | 1.00 | 72.94 | O |
| ATOM | 2680 | ND2 | ASN | A | 289 | −27.648 | −22.712 | 24.976 | 1.00 | 77.95 | N |
| ATOM | 2681 | C | ASN | A | 289 | −28.167 | −25.387 | 28.538 | 1.00 | 60.36 | C |
| ATOM | 2682 | O | ASN | A | 289 | −27.051 | −25.845 | 28.532 | 1.00 | 63.82 | O |
| ATOM | 2683 | N | ILE | A | 290 | −28.733 | −24.981 | 29.672 | 1.00 | 62.15 | N |
| ATOM | 2684 | CA | ILE | A | 290 | −28.009 | −25.013 | 30.951 | 1.00 | 60.71 | C |
| ATOM | 2685 | CB | ILE | A | 290 | −28.796 | −24.284 | 32.073 | 1.00 | 61.84 | C |
| ATOM | 2686 | CG1 | ILE | A | 290 | −28.857 | −22.779 | 31.808 | 1.00 | 54.64 | C |
| ATOM | 2687 | CD1 | ILE | A | 290 | −29.786 | −22.041 | 32.746 | 1.00 | 47.64 | C |
| ATOM | 2688 | CG2 | ILE | A | 290 | −28.165 | −24.534 | 33.435 | 1.00 | 56.15 | C |
| ATOM | 2689 | C | ILE | A | 290 | −27.705 | −26.430 | 31.400 | 1.00 | 62.05 | C |
| ATOM | 2690 | O | ILE | A | 290 | −26.621 | −26.740 | 31.856 | 1.00 | 65.78 | O |
| ATOM | 2691 | N | VAL | A | 291 | −28.697 | −27.297 | 31.301 | 1.00 | 64.73 | N |
| ATOM | 2692 | CA | VAL | A | 291 | −28.532 | −28.675 | 31.704 | 1.00 | 67.61 | C |
| ATOM | 2693 | CB | VAL | A | 291 | −29.859 | −29.462 | 31.646 | 1.00 | 65.42 | C |
| ATOM | 2694 | CG1 | VAL | A | 291 | −29.615 | −30.953 | 31.842 | 1.00 | 58.14 | C |
| ATOM | 2695 | CG2 | VAL | A | 291 | −30.817 | −28.938 | 32.703 | 1.00 | 57.07 | C |
| ATOM | 2696 | C | VAL | A | 291 | −27.448 | −29.358 | 30.872 | 1.00 | 66.55 | C |
| ATOM | 2697 | O | VAL | A | 291 | −26.585 | −30.038 | 31.402 | 1.00 | 65.35 | O |
| ATOM | 2698 | N | ARG | A | 292 | −27.488 | −29.134 | 29.561 | 1.00 | 64.78 | N |
| ATOM | 2699 | CA | ARG | A | 292 | −26.499 | −29.703 | 28.665 | 1.00 | 66.27 | C |
| ATOM | 2700 | CB | ARG | A | 292 | −26.839 | −29.335 | 27.226 | 1.00 | 64.83 | C |
| ATOM | 2701 | CG | ARG | A | 292 | −25.825 | −29.820 | 26.222 | 1.00 | 59.47 | C |
| ATOM | 2702 | CD | ARG | A | 292 | −25.963 | −29.060 | 24.920 | 1.00 | 69.79 | C |
| ATOM | 2703 | NE | ARG | A | 292 | −25.669 | −27.640 | 25.099 | 1.00 | 70.30 | N |
| ATOM | 2704 | CZ | ARG | A | 292 | −24.464 | −27.103 | 24.931 | 1.00 | 77.24 | C |
| ATOM | 2705 | NH1 | ARG | A | 292 | −23.443 | −27.869 | 24.573 | 1.00 | 86.12 | N |
| ATOM | 2706 | NH2 | ARG | A | 292 | −24.277 | −25.802 | 25.118 | 1.00 | 71.29 | N |
| ATOM | 2707 | C | ARG | A | 292 | −25.086 | −29.243 | 28.974 | 1.00 | 68.41 | C |
| ATOM | 2708 | O | ARG | A | 292 | −24.188 | −30.048 | 29.045 | 1.00 | 72.04 | O |
| ATOM | 2709 | N | ILE | A | 293 | −24.907 | −27.947 | 29.225 | 1.00 | 68.29 | N |
| ATOM | 2710 | CA | ILE | A | 293 | −23.623 | −27.418 | 29.636 | 1.00 | 65.12 | C |
| ATOM | 2711 | CB | ILE | A | 293 | −23.627 | −25.893 | 29.798 | 1.00 | 62.43 | C |
| ATOM | 2712 | CG1 | ILE | A | 293 | −23.892 | −25.229 | 28.445 | 1.00 | 60.54 | C |
| ATOM | 2713 | CD1 | ILE | A | 293 | −23.778 | −23.721 | 28.463 | 1.00 | 57.55 | C |
| ATOM | 2714 | CG2 | ILE | A | 293 | −22.292 | −25.426 | 30.345 | 1.00 | 59.73 | C |
| ATOM | 2715 | C | ILE | A | 293 | −23.123 | −28.103 | 30.891 | 1.00 | 72.40 | C |
| ATOM | 2716 | O | ILE | A | 293 | −21.980 | −28.506 | 30.959 | 1.00 | 82.46 | O |
| ATOM | 2717 | N | LEU | A | 294 | −24.003 | −28.288 | 31.868 | 1.00 | 71.87 | N |
| ATOM | 2718 | CA | LEU | A | 294 | −23.619 | −28.934 | 33.109 | 1.00 | 75.56 | C |
| ATOM | 2719 | CB | LEU | A | 294 | −24.784 | −28.965 | 34.097 | 1.00 | 79.59 | C |
| ATOM | 2720 | CG | LEU | A | 294 | −24.940 | −27.714 | 34.964 | 1.00 | 79.14 | C |
| ATOM | 2721 | CD1 | LEU | A | 294 | −26.016 | −27.926 | 36.014 | 1.00 | 80.10 | C |
| ATOM | 2722 | CD2 | LEU | A | 294 | −23.615 | −27.345 | 35.616 | 1.00 | 83.80 | C |
| ATOM | 2723 | C | LEU | A | 294 | −23.078 | −30.357 | 32.873 | 1.00 | 77.63 | C |
| ATOM | 2724 | O | LEU | A | 294 | −22.029 | −30.691 | 33.391 | 1.00 | 81.02 | O |
| ATOM | 2725 | N | MET | A | 295 | −23.713 | −31.146 | 31.990 | 1.00 | 78.82 | N |
| ATOM | 2726 | CA | MET | A | 295 | −23.110 | −32.426 | 31.590 | 1.00 | 85.23 | C |
| ATOM | 2727 | CB | MET | A | 295 | −24.133 | −33.265 | 30.818 | 1.00 | 82.35 | C |
| ATOM | 2728 | CG | MET | A | 295 | −25.393 | −33.617 | 31.597 | 1.00 | 78.74 | C |
| ATOM | 2729 | SD | MET | A | 295 | −26.605 | −34.490 | 30.582 | 1.00 | 87.70 | S |
| ATOM | 2730 | CE | MET | A | 295 | −27.781 | −35.006 | 31.829 | 1.00 | 81.13 | C |
| ATOM | 2731 | C | MET | A | 295 | −21.843 | −32.235 | 30.709 | 1.00 | 83.74 | C |
| ATOM | 2732 | O | MET | A | 295 | −20.888 | −32.987 | 30.777 | 1.00 | 87.43 | O |
| ATOM | 2733 | N | THR | A | 296 | −21.970 | −31.351 | 29.721 | 1.00 | 78.53 | N |
| ATOM | 2734 | CA | THR | A | 296 | −21.000 | −31.261 | 28.636 | 1.00 | 79.96 | C |
| ATOM | 2735 | CB | THR | A | 296 | −21.515 | −30.486 | 27.408 | 1.00 | 75.80 | C |
| ATOM | 2736 | OG1 | THR | A | 296 | −22.008 | −29.210 | 27.816 | 1.00 | 79.37 | O |
| ATOM | 2737 | CG2 | THR | A | 296 | −22.628 | −31.265 | 26.721 | 1.00 | 71.22 | C |
| ATOM | 2738 | C | THR | A | 296 | −19.592 | −30.800 | 29.051 | 1.00 | 88.03 | C |
| ATOM | 2739 | O | THR | A | 296 | −18.607 | −31.411 | 28.642 | 1.00 | 93.90 | O |
| ATOM | 2740 | N | LYS | A | 297 | −19.484 | −29.722 | 29.838 | 1.00 | 80.35 | N |
| ATOM | 2741 | CA | LYS | A | 297 | −18.150 | −29.227 | 30.161 | 1.00 | 84.03 | C |
| ATOM | 2742 | CB | LYS | A | 297 | −17.958 | −27.800 | 29.636 | 1.00 | 85.79 | C |
| ATOM | 2743 | CG | LYS | A | 297 | −17.981 | −27.643 | 28.125 | 1.00 | 87.57 | C |
| ATOM | 2744 | CD | LYS | A | 297 | −17.700 | −26.191 | 27.745 | 1.00 | 91.44 | C |
| ATOM | 2745 | CE | LYS | A | 297 | −17.643 | −25.992 | 26.237 | 1.00 | 99.83 | C |
| ATOM | 2746 | NZ | LYS | A | 297 | −17.262 | −24.594 | 25.870 | 1.00 | 79.91 | N |
| ATOM | 2747 | C | LYS | A | 297 | −17.886 | −29.228 | 31.653 | 1.00 | 85.14 | C |
| ATOM | 2748 | O | LYS | A | 297 | −16.756 | −29.391 | 32.099 | 1.00 | 89.37 | O |
| ATOM | 2749 | N | LEU | A | 298 | −18.942 | −28.930 | 32.393 | 1.00 | 87.85 | N |
| ATOM | 2750 | CA | LEU | A | 298 | −18.876 | −28.679 | 33.822 | 1.00 | 91.92 | C |
| ATOM | 2751 | CB | LEU | A | 298 | −19.805 | −27.523 | 34.203 | 1.00 | 89.79 | C |
| ATOM | 2752 | CG | LEU | A | 298 | −19.449 | −26.210 | 33.497 | 1.00 | 80.67 | C |
| ATOM | 2753 | CD1 | LEU | A | 298 | −20.423 | −25.105 | 33.858 | 1.00 | 83.55 | C |
| ATOM | 2754 | CD2 | LEU | A | 298 | −18.020 | −25.794 | 33.819 | 1.00 | 77.04 | C |
| ATOM | 2755 | C | LEU | A | 298 | −19.116 | −29.904 | 34.704 | 1.00 | 92.47 | C |
| ATOM | 2756 | O | LEU | A | 298 | −19.695 | −29.794 | 35.781 | 1.00 | 101.97 | O |
| ATOM | 2757 | N | ARG | A | 299 | −18.703 | −31.086 | 34.258 | 1.00 | 91.54 | N |
| ATOM | 2758 | CA | ARG | A | 299 | −18.918 | −32.279 | 35.078 | 1.00 | 96.98 | C |
| ATOM | 2759 | CB | ARG | A | 299 | −18.576 | −33.542 | 34.291 | 1.00 | 102.54 | C |
| ATOM | 2760 | CG | ARG | A | 299 | −19.603 | −34.045 | 33.308 | 1.00 | 98.12 | C |
| ATOM | 2761 | CD | ARG | A | 299 | −19.047 | −35.292 | 32.627 | 1.00 | 106.76 | C |
| ATOM | 2762 | NE | ARG | A | 299 | −19.826 | −35.736 | 31.474 | 1.00 | 116.01 | N |
| ATOM | 2763 | CZ | ARG | A | 299 | −19.515 | −36.800 | 30.738 | 1.00 | 110.76 | C |
| ATOM | 2764 | NH1 | ARG | A | 299 | −18.445 | −37.523 | 31.039 | 1.00 | 112.21 | N |
| ATOM | 2765 | NH2 | ARG | A | 299 | −20.272 | −37.143 | 29.703 | 1.00 | 103.87 | N |
| ATOM | 2766 | C | ARG | A | 299 | −17.947 | −32.228 | 36.233 | 1.00 | 104.94 | C |
| ATOM | 2767 | O | ARG | A | 299 | −18.294 | −32.495 | 37.382 | 1.00 | 107.63 | O |
| ATOM | 2768 | N | ALA | A | 300 | −16.685 | −32.077 | 35.846 | 1.00 | 102.25 | N |
| ATOM | 2769 | CA | ALA | A | 300 | −15.581 | −32.211 | 36.755 | 1.00 | 99.97 | C |
| ATOM | 2770 | CB | ALA | A | 300 | −14.385 | −32.838 | 36.051 | 1.00 | 94.16 | C |
| ATOM | 2771 | C | ALA | A | 300 | −15.186 | −30.869 | 37.383 | 1.00 | 105.26 | C |
| ATOM | 2772 | O | ALA | A | 300 | −14.128 | −30.783 | 38.011 | 1.00 | 109.00 | O |
| ATOM | 2773 | N | SER | A | 301 | −15.934 | −29.789 | 37.038 | 1.00 | 104.47 | N |
| ATOM | 2774 | CA | SER | A | 301 | −15.481 | −28.420 | 37.348 | 1.00 | 110.37 | C |
| ATOM | 2775 | CB | SER | A | 301 | −16.352 | −27.388 | 36.627 | 1.00 | 97.17 | C |
| ATOM | 2776 | OG | SER | A | 301 | −17.694 | −27.456 | 37.068 | 1.00 | 90.67 | O |
| ATOM | 2777 | C | SER | A | 301 | −15.421 | −28.086 | 38.842 | 1.00 | 108.87 | C |
| ATOM | 2778 | O | SER | A | 301 | −14.410 | −27.555 | 39.309 | 1.00 | 108.90 | O |
| ATOM | 2779 | N | THR | A | 302 | −16.532 | −28.344 | 39.552 | 1.00 | 103.72 | N |
| ATOM | 2780 | CA | THR | A | 302 | −16.710 | −27.974 | 40.980 | 1.00 | 105.26 | C |
| ATOM | 2781 | CB | THR | A | 302 | −16.251 | −29.104 | 41.929 | 1.00 | 98.97 | C |
| ATOM | 2782 | OG1 | THR | A | 302 | −14.913 | −29.499 | 41.602 | 1.00 | 97.45 | O |
| ATOM | 2783 | CG2 | THR | A | 302 | −17.172 | −30.308 | 41.807 | 1.00 | 96.12 | C |
| ATOM | 2784 | C | THR | A | 302 | −16.045 | −26.618 | 41.401 | 1.00 | 101.87 | C |
| ATOM | 2785 | O | THR | A | 302 | −15.208 | −26.562 | 42.302 | 1.00 | 99.71 | O |
| ATOM | 2786 | O | THR | A | 303 | −18.338 | −24.083 | 42.127 | 1.00 | 93.28 | O |
| ATOM | 2787 | N | THR | A | 303 | −16.531 | −25.518 | 40.801 | 1.00 | 96.15 | N |
| ATOM | 2788 | CA | THR | A | 303 | −16.132 | −24.177 | 41.189 | 1.00 | 92.84 | C |
| ATOM | 2789 | C | THR | A | 303 | −17.237 | −23.556 | 42.032 | 1.00 | 94.65 | C |
| ATOM | 2790 | CB | THR | A | 303 | −15.848 | −23.282 | 39.965 | 1.00 | 90.66 | C |
| ATOM | 2791 | OG1 | THR | A | 303 | −17.003 | −23.235 | 39.118 | 1.00 | 98.07 | O |
| ATOM | 2792 | CG2 | THR | A | 303 | −14.676 | −23.827 | 39.172 | 1.00 | 107.38 | C |
| ATOM | 2793 | O | SER | A | 304 | −20.319 | −21.609 | 43.077 | 1.00 | 95.23 | O |
| ATOM | 2794 | N | SER | A | 304 | −16.945 | −22.407 | 42.626 | 1.00 | 93.69 | N |
| ATOM | 2795 | CA | SER | A | 304 | −17.933 | −21.726 | 43.430 | 1.00 | 94.07 | C |
| ATOM | 2796 | C | SER | A | 304 | −19.201 | −21.420 | 42.609 | 1.00 | 95.60 | C |
| ATOM | 2797 | CB | SER | A | 304 | −17.354 | −20.442 | 44.032 | 1.00 | 89.62 | C |
| ATOM | 2798 | OG | SER | A | 304 | −16.765 | −19.626 | 43.036 | 1.00 | 99.78 | O |
| ATOM | 2799 | N | GLU | A | 305 | −19.003 | −21.004 | 41.348 | 1.00 | 96.81 | N |
| ATOM | 2800 | CA | GLU | A | 305 | −20.128 | −20.721 | 40.469 | 1.00 | 91.03 | C |
| ATOM | 2801 | CB | GLU | A | 305 | −19.635 | −19.973 | 39.225 | 1.00 | 84.69 | C |
| ATOM | 2802 | CG | GLU | A | 305 | −20.718 | −19.628 | 38.211 | 1.00 | 93.58 | C |
| ATOM | 2803 | CD | GLU | A | 305 | −21.649 | −18.525 | 38.687 | 1.00 | 93.00 | C |
| ATOM | 2804 | OE1 | GLU | A | 305 | −21.264 | −17.338 | 38.602 | 1.00 | 77.63 | O |
| ATOM | 2805 | OE2 | GLU | A | 305 | −22.768 | −18.844 | 39.141 | 1.00 | 96.01 | O |
| ATOM | 2806 | C | GLU | A | 305 | −20.915 | −21.978 | 40.051 | 1.00 | 86.30 | C |
| ATOM | 2807 | O | GLU | A | 305 | −22.133 | −21.944 | 39.997 | 1.00 | 87.74 | O |
| ATOM | 2808 | N | THR | A | 306 | −20.212 | −23.052 | 39.673 | 1.00 | 85.71 | N |
| ATOM | 2809 | CA | THR | A | 306 | −20.886 | −24.246 | 39.141 | 1.00 | 88.58 | C |
| ATOM | 2810 | CB | THR | A | 306 | −19.887 | −25.243 | 38.504 | 1.00 | 93.26 | C |
| ATOM | 2811 | OG1 | THR | A | 306 | −19.153 | −24.596 | 37.457 | 1.00 | 86.92 | O |
| ATOM | 2812 | CG2 | THR | A | 306 | −20.627 | −26.447 | 37.929 | 1.00 | 88.19 | C |
| ATOM | 2813 | C | THR | A | 306 | −21.734 | −24.985 | 40.174 | 1.00 | 83.04 | C |
| ATOM | 2814 | O | THR | A | 306 | −22.828 | −25.451 | 39.881 | 1.00 | 79.53 | O |
| ATOM | 2815 | N | ILE | A | 307 | −21.233 | −25.040 | 41.409 | 1.00 | 84.69 | N |
| ATOM | 2816 | CA | ILE | A | 307 | −21.982 | −25.612 | 42.506 | 1.00 | 83.23 | C |
| ATOM | 2817 | CB | ILE | A | 307 | −21.185 | −25.589 | 43.818 | 1.00 | 84.83 | C |
| ATOM | 2818 | CG1 | ILE | A | 307 | −20.026 | −26.585 | 43.731 | 1.00 | 87.11 | C |
| ATOM | 2819 | CD1 | ILE | A | 307 | −19.383 | −26.896 | 45.066 | 1.00 | 90.81 | C |
| ATOM | 2820 | CG2 | ILE | A | 307 | −22.086 | −25.925 | 44.998 | 1.00 | 77.77 | C |
| ATOM | 2821 | C | ILE | A | 307 | −23.317 | −24.900 | 42.680 | 1.00 | 84.14 | C |
| ATOM | 2822 | O | ILE | A | 307 | −24.356 | −25.529 | 42.750 | 1.00 | 84.07 | O |
| ATOM | 2823 | N | GLN | A | 308 | −23.285 | −23.563 | 42.692 | 1.00 | 86.85 | N |
| ATOM | 2824 | CA | GLN | A | 308 | −24.509 | −22.786 | 42.844 | 1.00 | 78.56 | C |
| ATOM | 2825 | CB | GLN | A | 308 | −24.199 | −21.291 | 42.925 | 1.00 | 79.62 | C |
| ATOM | 2826 | CG | GLN | A | 308 | −23.479 | −20.853 | 44.187 | 1.00 | 96.11 | C |
| ATOM | 2827 | CD | GLN | A | 308 | −23.091 | −19.385 | 44.143 | 1.00 | 103.10 | C |
| ATOM | 2828 | OE1 | GLN | A | 308 | −23.009 | −18.792 | 43.067 | 1.00 | 98.32 | O |
| ATOM | 2829 | NE2 | GLN | A | 308 | −22.858 | −18.790 | 45.311 | 1.00 | 100.94 | N |
| ATOM | 2830 | C | GLN | A | 308 | −25.456 | −23.005 | 41.686 | 1.00 | 74.37 | C |
| ATOM | 2831 | O | GLN | A | 308 | −26.646 | −23.159 | 41.861 | 1.00 | 74.15 | O |
| ATOM | 2832 | N | ALA | A | 309 | −24.925 | −23.035 | 40.491 | 1.00 | 75.88 | N |
| ATOM | 2833 | CA | ALA | A | 309 | −25.745 | −23.252 | 39.332 | 1.00 | 70.38 | C |
| ATOM | 2834 | CB | ALA | A | 309 | −24.909 | −23.159 | 38.060 | 1.00 | 70.41 | C |
| ATOM | 2835 | C | ALA | A | 309 | −26.481 | −24.584 | 39.390 | 1.00 | 71.93 | C |
| ATOM | 2836 | O | ALA | A | 309 | −27.645 | −24.666 | 39.088 | 1.00 | 68.45 | O |
| ATOM | 2837 | N | ARG | A | 310 | −25.786 | −25.634 | 39.811 | 1.00 | 83.45 | N |
| ATOM | 2838 | CA | ARG | A | 310 | −26.418 | −26.938 | 40.051 | 1.00 | 79.10 | C |
| ATOM | 2839 | CB | ARG | A | 310 | −25.379 | −27.969 | 40.505 | 1.00 | 83.70 | C |
| ATOM | 2840 | CG | ARG | A | 310 | −24.413 | −28.395 | 39.408 | 1.00 | 93.77 | C |
| ATOM | 2841 | CD | ARG | A | 310 | −23.445 | −29.461 | 39.892 | 1.00 | 101.51 | C |
| ATOM | 2842 | NE | ARG | A | 310 | −22.858 | −30.203 | 38.777 | 1.00 | 106.37 | N |
| ATOM | 2843 | CZ | ARG | A | 310 | −21.986 | −31.198 | 38.915 | 1.00 | 110.64 | C |
| ATOM | 2844 | NH1 | ARG | A | 310 | −21.589 | −31.571 | 40.124 | 1.00 | 109.31 | N |
| ATOM | 2845 | NH2 | ARG | A | 310 | −21.510 | −31.823 | 37.845 | 1.00 | 106.51 | N |
| ATOM | 2846 | C | ARG | A | 310 | −27.545 | −26.836 | 41.066 | 1.00 | 67.85 | C |
| ATOM | 2847 | O | ARG | A | 310 | −28.652 | −27.275 | 40.841 | 1.00 | 64.99 | O |
| ATOM | 2848 | N | LYS | A | 311 | −27.232 | −26.235 | 42.197 | 1.00 | 68.71 | N |
| ATOM | 2849 | CA | LYS | A | 311 | −28.185 | −26.066 | 43.268 | 1.00 | 69.89 | C |
| ATOM | 2850 | CB | LYS | A | 311 | −27.564 | −25.315 | 44.452 | 1.00 | 74.29 | C |
| ATOM | 2851 | CG | LYS | A | 311 | −28.403 | −25.385 | 45.725 | 1.00 | 77.22 | C |
| ATOM | 2852 | CD | LYS | A | 311 | −27.711 | −24.738 | 46.918 | 1.00 | 70.76 | C |
| ATOM | 2853 | CE | LYS | A | 311 | −27.721 | −23.222 | 46.817 | 1.00 | 67.87 | C |
| ATOM | 2854 | NZ | LYS | A | 311 | −27.154 | −22.575 | 48.034 | 1.00 | 68.21 | N |
| ATOM | 2855 | C | LYS | A | 311 | −29.428 | −25.353 | 42.776 | 1.00 | 68.15 | C |
| ATOM | 2856 | O | LYS | A | 311 | −30.529 | −25.761 | 43.068 | 1.00 | 71.85 | O |
| ATOM | 2857 | N | ALA | A | 312 | −29.228 | −24.326 | 41.956 | 1.00 | 68.31 | N |
| ATOM | 2858 | CA | ALA | A | 312 | −30.326 | −23.646 | 41.298 | 1.00 | 59.04 | C |
| ATOM | 2859 | CB | ALA | A | 312 | −29.824 | −22.415 | 40.561 | 1.00 | 66.28 | C |
| ATOM | 2860 | C | ALA | A | 312 | −31.110 | −24.539 | 40.363 | 1.00 | 55.56 | C |
| ATOM | 2861 | O | ALA | A | 312 | −32.306 | −24.572 | 40.430 | 1.00 | 62.12 | O |
| ATOM | 2862 | N | VAL | A | 313 | −30.437 | −25.238 | 39.462 | 1.00 | 61.65 | N |
| ATOM | 2863 | CA | VAL | A | 313 | −31.123 | −26.117 | 38.515 | 1.00 | 67.57 | C |
| ATOM | 2864 | CB | VAL | A | 313 | −30.113 | −26.751 | 37.526 | 1.00 | 72.66 | C |
| ATOM | 2865 | CG1 | VAL | A | 313 | −30.714 | −27.963 | 36.814 | 1.00 | 75.17 | C |
| ATOM | 2866 | CG2 | VAL | A | 313 | −29.637 | −25.714 | 36.524 | 1.00 | 70.04 | C |
| ATOM | 2867 | C | VAL | A | 313 | −31.924 | −27.227 | 39.206 | 1.00 | 68.89 | C |
| ATOM | 2868 | O | VAL | A | 313 | −33.048 | −27.549 | 38.840 | 1.00 | 64.85 | O |
| ATOM | 2869 | N | LYS | A | 314 | −31.359 | −27.757 | 40.276 | 1.00 | 64.40 | N |
| ATOM | 2870 | CA | LYS | A | 314 | −32.040 | −28.743 | 41.068 | 1.00 | 65.32 | C |
| ATOM | 2871 | CB | LYS | A | 314 | −31.160 | −29.226 | 42.224 | 1.00 | 71.21 | C |
| ATOM | 2872 | CG | LYS | A | 314 | −30.044 | −30.180 | 41.834 | 1.00 | 69.92 | C |
| ATOM | 2873 | CD | LYS | A | 314 | −29.244 | −30.584 | 43.063 | 1.00 | 67.31 | C |
| ATOM | 2874 | CE | LYS | A | 314 | −28.028 | −31.419 | 42.701 | 1.00 | 74.34 | C |
| ATOM | 2875 | NZ | LYS | A | 314 | −27.188 | −31.701 | 43.901 | 1.00 | 70.61 | N |
| ATOM | 2876 | C | LYS | A | 314 | −33.327 | −28.170 | 41.623 | 1.00 | 65.14 | C |
| ATOM | 2877 | O | LYS | A | 314 | −34.360 | −28.804 | 41.571 | 1.00 | 69.28 | O |
| ATOM | 2878 | N | ALA | A | 315 | −33.235 | −26.973 | 42.199 | 1.00 | 62.34 | N |
| ATOM | 2879 | CA | ALA | A | 315 | −34.392 | −26.312 | 42.739 | 1.00 | 58.70 | C |
| ATOM | 2880 | CB | ALA | A | 315 | −33.965 | −25.094 | 43.560 | 1.00 | 52.65 | C |
| ATOM | 2881 | C | ALA | A | 315 | −35.423 | −25.908 | 41.681 | 1.00 | 60.05 | C |
| ATOM | 2882 | O | ALA | A | 315 | −36.601 | −26.095 | 41.886 | 1.00 | 62.00 | O |
| ATOM | 2883 | N | THR | A | 316 | −34.980 | −25.425 | 40.520 | 1.00 | 57.96 | N |
| ATOM | 2884 | CA | THR | A | 316 | −35.878 | −25.095 | 39.406 | 1.00 | 58.69 | C |
| ATOM | 2885 | CB | THR | A | 316 | −35.129 | −24.521 | 38.182 | 1.00 | 56.15 | C |
| ATOM | 2886 | OG1 | THR | A | 316 | −34.550 | −23.258 | 38.525 | 1.00 | 53.89 | O |
| ATOM | 2887 | CG2 | THR | A | 316 | −36.091 | −24.315 | 37.016 | 1.00 | 54.45 | C |
| ATOM | 2888 | C | THR | A | 316 | −36.729 | −26.273 | 38.990 | 1.00 | 64.13 | C |
| ATOM | 2889 | O | THR | A | 316 | −37.927 | −26.146 | 38.821 | 1.00 | 60.60 | O |
| ATOM | 2890 | N | LEU | A | 317 | −36.071 | −27.434 | 38.870 | 1.00 | 65.62 | N |
| ATOM | 2891 | CA | LEU | A | 317 | −36.730 | −28.678 | 38.530 | 1.00 | 67.37 | C |
| ATOM | 2892 | CB | LEU | A | 317 | −35.715 | −29.806 | 38.350 | 1.00 | 70.28 | C |
| ATOM | 2893 | CG | LEU | A | 317 | −34.865 | −29.719 | 37.082 | 1.00 | 71.49 | C |
| ATOM | 2894 | CD1 | LEU | A | 317 | −33.873 | −30.865 | 37.035 | 1.00 | 74.28 | C |
| ATOM | 2895 | CD2 | LEU | A | 317 | −35.748 | −29.716 | 35.839 | 1.00 | 68.11 | C |
| ATOM | 2896 | C | LEU | A | 317 | −37.768 | −29.050 | 39.574 | 1.00 | 68.71 | C |
| ATOM | 2897 | O | LEU | A | 317 | −38.841 | −29.485 | 39.230 | 1.00 | 75.78 | O |
| ATOM | 2898 | N | VAL | A | 318 | −37.460 | −28.836 | 40.853 | 1.00 | 62.75 | N |
| ATOM | 2899 | CA | VAL | A | 318 | −38.439 | −29.037 | 41.914 | 1.00 | 63.18 | C |
| ATOM | 2900 | CB | VAL | A | 318 | −37.791 | −28.967 | 43.313 | 1.00 | 68.12 | C |
| ATOM | 2901 | CG1 | VAL | A | 318 | −38.839 | −29.186 | 44.399 | 1.00 | 60.59 | C |
| ATOM | 2902 | CG2 | VAL | A | 318 | −36.676 | −29.994 | 43.432 | 1.00 | 65.32 | C |
| ATOM | 2903 | C | VAL | A | 318 | −39.619 | −28.033 | 41.841 | 1.00 | 64.49 | C |
| ATOM | 2904 | O | VAL | A | 318 | −40.773 | −28.389 | 41.976 | 1.00 | 62.34 | O |
| ATOM | 2905 | N | LEU | A | 319 | −39.303 | −26.769 | 41.597 | 1.00 | 65.40 | N |
| ATOM | 2906 | CA | LEU | A | 319 | −40.297 | −25.694 | 41.513 | 1.00 | 60.96 | C |
| ATOM | 2907 | CB | LEU | A | 319 | −39.604 | −24.331 | 41.446 | 1.00 | 54.53 | C |
| ATOM | 2908 | CG | LEU | A | 319 | −40.507 | −23.097 | 41.438 | 1.00 | 48.36 | C |
| ATOM | 2909 | CD1 | LEU | A | 319 | −41.138 | −22.889 | 42.804 | 1.00 | 53.90 | C |
| ATOM | 2910 | CD2 | LEU | A | 319 | −39.734 | −21.865 | 41.008 | 1.00 | 50.05 | C |
| ATOM | 2911 | C | LEU | A | 319 | −41.227 | −25.847 | 40.315 | 1.00 | 62.99 | C |
| ATOM | 2912 | O | LEU | A | 319 | −42.401 | −25.538 | 40.367 | 1.00 | 68.53 | O |
| ATOM | 2913 | N | LEU | A | 320 | −40.665 | −26.264 | 39.201 | 1.00 | 63.11 | N |
| ATOM | 2914 | CA | LEU | A | 320 | −41.354 | −26.241 | 37.933 | 1.00 | 67.82 | C |
| ATOM | 2915 | CB | LEU | A | 320 | −40.388 | −26.633 | 36.810 | 1.00 | 62.21 | C |
| ATOM | 2916 | CG | LEU | A | 320 | −40.951 | −26.803 | 35.402 | 1.00 | 62.66 | C |
| ATOM | 2917 | CD1 | LEU | A | 320 | −41.340 | −25.456 | 34.812 | 1.00 | 59.66 | C |
| ATOM | 2918 | CD2 | LEU | A | 320 | −39.938 | −27.520 | 34.521 | 1.00 | 66.80 | C |
| ATOM | 2919 | C | LEU | A | 320 | −42.606 | −27.141 | 37.921 | 1.00 | 72.95 | C |
| ATOM | 2920 | O | LEU | A | 320 | −43.647 | −26.651 | 37.543 | 1.00 | 72.56 | O |
| ATOM | 2921 | N | PRO | A | 321 | −42.686 | −28.418 | 38.347 | 1.00 | 73.70 | N |
| ATOM | 2922 | CA | PRO | A | 321 | −43.957 | −29.118 | 38.435 | 1.00 | 63.10 | C |
| ATOM | 2923 | CB | PRO | A | 321 | −43.562 | −30.484 | 38.996 | 1.00 | 65.46 | C |
| ATOM | 2924 | CG | PRO | A | 321 | −42.204 | −30.695 | 38.489 | 1.00 | 70.78 | C |
| ATOM | 2925 | CD | PRO | A | 321 | −41.566 | −29.341 | 38.554 | 1.00 | 65.75 | C |
| ATOM | 2926 | C | PRO | A | 321 | −44.970 | −28.460 | 39.361 | 1.00 | 63.33 | C |
| ATOM | 2927 | O | PRO | A | 321 | −46.132 | −28.442 | 39.022 | 1.00 | 73.29 | O |
| ATOM | 2928 | N | LEU | A | 322 | −44.572 | −27.954 | 40.519 | 1.00 | 63.14 | N |
| ATOM | 2929 | CA | LEU | A | 322 | −45.521 | −27.343 | 41.437 | 1.00 | 67.35 | C |
| ATOM | 2930 | CB | LEU | A | 322 | −44.807 | −26.809 | 42.681 | 1.00 | 63.05 | C |
| ATOM | 2931 | CG | LEU | A | 322 | −44.146 | −27.848 | 43.584 | 1.00 | 59.92 | C |
| ATOM | 2932 | CD1 | LEU | A | 322 | −43.447 | −27.161 | 44.740 | 1.00 | 63.48 | C |
| ATOM | 2933 | CD2 | LEU | A | 322 | −45.171 | −28.846 | 44.095 | 1.00 | 48.46 | C |
| ATOM | 2934 | C | LEU | A | 322 | −46.363 | −26.235 | 40.802 | 1.00 | 69.50 | C |
| ATOM | 2935 | O | LEU | A | 322 | −47.574 | −26.164 | 40.985 | 1.00 | 73.27 | O |
| ATOM | 2936 | N | LEU | A | 323 | −45.701 | −25.341 | 40.069 | 1.00 | 62.19 | N |
| ATOM | 2937 | CA | LEU | A | 323 | −46.404 | −24.236 | 39.471 | 1.00 | 67.12 | C |
| ATOM | 2938 | CB | LEU | A | 323 | −45.456 | −23.053 | 39.248 | 1.00 | 65.21 | C |
| ATOM | 2939 | CG | LEU | A | 323 | −44.661 | −22.568 | 40.467 | 1.00 | 58.35 | C |
| ATOM | 2940 | CD1 | LEU | A | 323 | −43.924 | −21.276 | 40.154 | 1.00 | 57.37 | C |
| ATOM | 2941 | CD2 | LEU | A | 323 | −45.562 | −22.381 | 41.676 | 1.00 | 58.12 | C |
| ATOM | 2942 | C | LEU | A | 323 | −47.055 | −24.666 | 38.158 | 1.00 | 75.73 | C |
| ATOM | 2943 | O | LEU | A | 323 | −48.181 | −24.305 | 37.837 | 1.00 | 81.53 | O |
| ATOM | 2944 | N | GLY | A | 324 | −46.351 | −25.527 | 37.444 | 1.00 | 76.75 | N |
| ATOM | 2945 | CA | GLY | A | 324 | −46.777 | −26.053 | 36.166 | 1.00 | 78.95 | C |
| ATOM | 2946 | C | GLY | A | 324 | −48.007 | −26.914 | 36.131 | 1.00 | 82.73 | C |
| ATOM | 2947 | O | GLY | A | 324 | −48.734 | −26.857 | 35.147 | 1.00 | 85.51 | O |
| ATOM | 2948 | N | ILE | A | 325 | −48.229 | −27.757 | 37.152 | 1.00 | 81.96 | N |
| ATOM | 2949 | CA | ILE | A | 325 | −49.378 | −28.663 | 37.166 | 1.00 | 84.02 | C |
| ATOM | 2950 | CB | ILE | A | 325 | −49.324 | −29.638 | 38.353 | 1.00 | 75.83 | C |
| ATOM | 2951 | CG1 | ILE | A | 325 | −50.541 | −30.562 | 38.319 | 1.00 | 81.72 | C |
| ATOM | 2952 | CD1 | ILE | A | 325 | −50.428 | −31.763 | 39.232 | 1.00 | 83.95 | C |
| ATOM | 2953 | CG2 | ILE | A | 325 | −49.153 | −28.890 | 39.675 | 1.00 | 74.22 | C |
| ATOM | 2954 | C | ILE | A | 325 | −50.660 | −27.866 | 37.084 | 1.00 | 90.58 | C |
| ATOM | 2955 | O | ILE | A | 325 | −51.615 | −28.247 | 36.430 | 1.00 | 90.46 | O |
| ATOM | 2956 | N | THR | A | 326 | −50.672 | −26.748 | 37.798 | 1.00 | 88.66 | N |
| ATOM | 2957 | CA | THR | A | 326 | −51.850 | −25.927 | 37.882 | 1.00 | 82.86 | C |
| ATOM | 2958 | CB | THR | A | 326 | −51.620 | −24.693 | 38.775 | 1.00 | 80.91 | C |
| ATOM | 2959 | OG1 | THR | A | 326 | −51.239 | −25.115 | 40.089 | 1.00 | 88.27 | O |
| ATOM | 2960 | CG2 | THR | A | 326 | −52.886 | −23.857 | 38.861 | 1.00 | 83.52 | C |
| ATOM | 2961 | C | THR | A | 326 | −52.298 | −25.459 | 36.504 | 1.00 | 87.08 | C |
| ATOM | 2962 | O | THR | A | 326 | −53.466 | −25.541 | 36.160 | 1.00 | 96.64 | O |
| ATOM | 2963 | N | TYR | A | 327 | −51.344 | −25.016 | 35.690 | 1.00 | 90.36 | N |
| ATOM | 2964 | CA | TYR | A | 327 | −51.646 | −24.710 | 34.292 | 1.00 | 88.25 | C |
| ATOM | 2965 | CB | TYR | A | 327 | −50.490 | −23.937 | 33.635 | 1.00 | 83.65 | C |
| ATOM | 2966 | CG | TYR | A | 327 | −50.185 | −22.665 | 34.411 | 1.00 | 71.68 | C |
| ATOM | 2967 | CD1 | TYR | A | 327 | −50.889 | −21.486 | 34.172 | 1.00 | 66.48 | C |
| ATOM | 2968 | CE1 | TYR | A | 327 | −50.637 | −20.331 | 34.912 | 1.00 | 62.34 | C |
| ATOM | 2969 | CZ | TYR | A | 327 | −49.687 | −20.353 | 35.914 | 1.00 | 60.62 | C |
| ATOM | 2970 | OH | TYR | A | 327 | −49.440 | −19.214 | 36.652 | 1.00 | 60.39 | O |
| ATOM | 2971 | CE2 | TYR | A | 327 | −48.990 | −21.516 | 36.181 | 1.00 | 60.63 | C |
| ATOM | 2972 | CD2 | TYR | A | 327 | −49.243 | −22.662 | 35.433 | 1.00 | 66.80 | C |
| ATOM | 2973 | C | TYR | A | 327 | −52.115 | −25.948 | 33.502 | 1.00 | 91.95 | C |
| ATOM | 2974 | O | TYR | A | 327 | −53.047 | −25.874 | 32.712 | 1.00 | 103.73 | O |
| ATOM | 2975 | N | MET | A | 328 | −51.468 | −27.096 | 33.712 | 1.00 | 90.15 | N |
| ATOM | 2976 | CA | MET | A | 328 | −51.899 | −28.324 | 33.031 | 1.00 | 96.14 | C |
| ATOM | 2977 | CB | MET | A | 328 | −50.966 | −29.487 | 33.381 | 1.00 | 100.57 | C |
| ATOM | 2978 | CG | MET | A | 328 | −49.529 | −29.313 | 32.910 | 1.00 | 98.09 | C |
| ATOM | 2979 | SD | MET | A | 328 | −49.414 | −28.948 | 31.148 | 1.00 | 92.32 | S |
| ATOM | 2980 | CE | MET | A | 328 | −47.962 | −29.892 | 30.693 | 1.00 | 71.38 | C |
| ATOM | 2981 | C | MET | A | 328 | −53.356 | −28.708 | 33.349 | 1.00 | 103.79 | C |
| ATOM | 2982 | O | MET | A | 328 | −54.155 | −29.029 | 32.453 | 1.00 | 110.84 | O |
| ATOM | 2983 | N | LEU | A | 329 | −53.707 | −28.585 | 34.631 | 1.00 | 103.97 | N |
| ATOM | 2984 | CA | LEU | A | 329 | −55.052 | −28.870 | 35.082 | 1.00 | 107.35 | C |
| ATOM | 2985 | CB | LEU | A | 329 | −55.143 | −28.816 | 36.610 | 1.00 | 106.50 | C |
| ATOM | 2986 | CG | LEU | A | 329 | −54.319 | −29.845 | 37.391 | 1.00 | 98.20 | C |
| ATOM | 2987 | CD1 | LEU | A | 329 | −54.656 | −29.798 | 38.880 | 1.00 | 87.46 | C |
| ATOM | 2988 | CD2 | LEU | A | 329 | −54.515 | −31.247 | 36.826 | 1.00 | 88.93 | C |
| ATOM | 2989 | C | LEU | A | 329 | −56.025 | −27.865 | 34.454 | 1.00 | 108.23 | C |
| ATOM | 2990 | O | LEU | A | 329 | −57.169 | −28.187 | 34.195 | 1.00 | 114.22 | O |
| ATOM | 2991 | N | ALA | A | 330 | −55.579 | −26.608 | 34.291 | 1.00 | 105.59 | N |
| ATOM | 2992 | CA | ALA | A | 330 | −56.418 | −25.583 | 33.653 | 1.00 | 107.13 | C |
| ATOM | 2993 | CB | ALA | A | 330 | −55.693 | −24.244 | 33.616 | 1.00 | 103.41 | C |
| ATOM | 2994 | C | ALA | A | 330 | −56.942 | −25.964 | 32.228 | 1.00 | 110.32 | C |
| ATOM | 2995 | O | ALA | A | 330 | −58.157 | −25.919 | 31.989 | 1.00 | 111.75 | O |
| ATOM | 2996 | N | PHE | A | 331 | −56.042 | −26.407 | 31.312 | 1.00 | 107.76 | N |
| ATOM | 2997 | CA | PHE | A | 331 | −56.491 | −26.864 | 29.957 | 1.00 | 111.73 | C |
| ATOM | 2998 | CB | PHE | A | 331 | −55.288 | −27.240 | 29.082 | 1.00 | 109.74 | C |
| ATOM | 2999 | CG | PHE | A | 331 | −54.259 | −26.147 | 28.987 | 1.00 | 108.00 | C |
| ATOM | 3000 | CD2 | PHE | A | 331 | −52.908 | −26.433 | 29.095 | 1.00 | 107.41 | C |
| ATOM | 3001 | CE2 | PHE | A | 331 | −51.965 | −25.426 | 29.036 | 1.00 | 109.01 | C |
| ATOM | 3002 | CZ | PHE | A | 331 | −52.365 | −24.113 | 28.878 | 1.00 | 107.82 | C |
| ATOM | 3003 | CE1 | PHE | A | 331 | −53.710 | −23.809 | 28.779 | 1.00 | 98.64 | C |
| ATOM | 3004 | CD1 | PHE | A | 331 | −54.650 | −24.823 | 28.839 | 1.00 | 101.71 | C |
| ATOM | 3005 | C | PHE | A | 331 | −57.570 | −27.986 | 29.994 | 1.00 | 116.58 | C |
| ATOM | 3006 | O | PHE | A | 331 | −58.634 | −27.872 | 29.388 | 1.00 | 119.94 | O |
| ATOM | 3007 | N | VAL | A | 332 | −57.246 | −29.094 | 30.696 | 1.00 | 115.32 | N |
| ATOM | 3008 | CA | VAL | A | 332 | −58.143 | −30.263 | 30.801 | 1.00 | 117.26 | C |
| ATOM | 3009 | CB | VAL | A | 332 | −57.352 | −31.583 | 30.975 | 1.00 | 118.70 | C |
| ATOM | 3010 | CG2 | VAL | A | 332 | −56.553 | −31.578 | 32.274 | 1.00 | 111.61 | C |
| ATOM | 3011 | CG1 | VAL | A | 332 | −56.430 | −31.803 | 29.786 | 1.00 | 119.53 | C |
| ATOM | 3012 | C | VAL | A | 332 | −59.173 | −30.082 | 31.953 | 1.00 | 121.34 | C |
| ATOM | 3013 | O | VAL | A | 332 | −58.827 | −29.608 | 33.026 | 1.00 | 122.83 | O |
| ATOM | 3014 | N | ASN | A | 333 | −60.468 | −30.385 | 31.715 | 1.00 | 123.62 | N |
| ATOM | 3015 | CA | ASN | A | 333 | −61.479 | −30.131 | 32.761 | 1.00 | 120.43 | C |
| ATOM | 3016 | CB | ASN | A | 333 | −62.396 | −28.956 | 32.388 | 1.00 | 122.49 | C |
| ATOM | 3017 | CG | ASN | A | 333 | −62.952 | −29.057 | 30.976 | 1.00 | 120.52 | C |
| ATOM | 3018 | OD1 | ASN | A | 333 | −64.023 | −29.624 | 30.757 | 1.00 | 117.34 | O |
| ATOM | 3019 | ND2 | ASN | A | 333 | −62.240 | −28.477 | 30.016 | 1.00 | 121.13 | N |
| ATOM | 3020 | C | ASN | A | 333 | −62.303 | −31.398 | 33.156 | 1.00 | 119.36 | C |
| ATOM | 3021 | O | ASN | A | 333 | −63.117 | −31.874 | 32.367 | 1.00 | 126.72 | O |
| ATOM | 3022 | N | GLU | A | 338 | −67.455 | −38.448 | 40.214 | 1.00 | 95.08 | N |
| ATOM | 3023 | CA | GLU | A | 338 | −66.596 | −39.293 | 40.979 | 1.00 | 113.57 | C |
| ATOM | 3024 | C | GLU | A | 338 | −66.080 | −38.493 | 42.168 | 1.00 | 127.95 | C |
| ATOM | 3025 | O | GLU | A | 338 | −65.753 | −37.320 | 42.037 | 1.00 | 129.90 | O |
| ATOM | 3026 | CB | GLU | A | 338 | −65.399 | −39.739 | 40.141 | 1.00 | 112.71 | C |
| ATOM | 3027 | CG | GLU | A | 338 | −65.669 | −40.854 | 39.154 | 1.00 | 110.76 | C |
| ATOM | 3028 | CD | GLU | A | 338 | −64.413 | −41.266 | 38.405 | 1.00 | 109.79 | C |
| ATOM | 3029 | OE1 | GLU | A | 338 | −63.744 | −40.380 | 37.832 | 1.00 | 106.62 | O |
| ATOM | 3030 | OE2 | GLU | A | 338 | −64.086 | −42.470 | 38.399 | 1.00 | 105.38 | O |
| ATOM | 3031 | N | VAL | A | 339 | −65.911 | −39.169 | 43.305 | 1.00 | 132.72 | N |
| ATOM | 3032 | CA | VAL | A | 339 | −65.164 | −38.598 | 44.437 | 1.00 | 127.53 | C |
| ATOM | 3033 | CB | VAL | A | 339 | −64.862 | −39.658 | 45.512 | 1.00 | 123.72 | C |
| ATOM | 3034 | CG1 | VAL | A | 339 | −64.087 | −39.041 | 46.669 | 1.00 | 121.34 | C |
| ATOM | 3035 | CG2 | VAL | A | 339 | −66.154 | −40.296 | 46.004 | 1.00 | 120.93 | C |
| ATOM | 3036 | C | VAL | A | 339 | −63.829 | −37.964 | 43.936 | 1.00 | 123.84 | C |
| ATOM | 3037 | O | VAL | A | 339 | −63.445 | −36.875 | 44.337 | 1.00 | 123.41 | O |
| ATOM | 3038 | N | SER | A | 340 | −63.176 | −38.638 | 42.978 | 1.00 | 123.14 | N |
| ATOM | 3039 | CA | SER | A | 340 | −61.958 | −38.110 | 42.349 | 1.00 | 121.98 | C |
| ATOM | 3040 | CB | SER | A | 340 | −61.420 | −39.083 | 41.294 | 1.00 | 120.48 | C |
| ATOM | 3041 | OG | SER | A | 340 | −62.251 | −39.119 | 40.147 | 1.00 | 117.86 | O |
| ATOM | 3042 | C | SER | A | 340 | −62.112 | −36.669 | 41.732 | 1.00 | 118.12 | C |
| ATOM | 3043 | O | SER | A | 340 | −61.158 | −35.895 | 41.738 | 1.00 | 117.70 | O |
| ATOM | 3044 | N | ARG | A | 341 | −63.325 | −36.291 | 41.257 | 1.00 | 116.75 | N |
| ATOM | 3045 | CA | ARG | A | 341 | −63.570 | −34.902 | 40.790 | 1.00 | 116.68 | C |
| ATOM | 3046 | CB | ARG | A | 341 | −64.988 | −34.734 | 40.245 | 1.00 | 116.25 | C |
| ATOM | 3047 | CG | ARG | A | 341 | −65.232 | −33.362 | 39.626 | 1.00 | 110.68 | C |
| ATOM | 3048 | CD | ARG | A | 341 | −66.588 | −32.788 | 40.005 | 1.00 | 108.63 | C |
| ATOM | 3049 | NE | ARG | A | 341 | −67.695 | −33.583 | 39.481 | 1.00 | 118.04 | N |
| ATOM | 3050 | CZ | ARG | A | 341 | −68.955 | −33.162 | 39.430 | 1.00 | 111.31 | C |
| ATOM | 3051 | NH1 | ARG | A | 341 | −69.264 | −31.948 | 39.865 | 1.00 | 108.25 | N |
| ATOM | 3052 | NH2 | ARG | A | 341 | −69.905 | −33.949 | 38.940 | 1.00 | 97.33 | N |
| ATOM | 3053 | C | ARG | A | 341 | −63.342 | −33.898 | 41.921 | 1.00 | 118.88 | C |
| ATOM | 3054 | O | ARG | A | 341 | −62.718 | −32.864 | 41.736 | 1.00 | 115.23 | O |
| ATOM | 3055 | N | VAL | A | 342 | −63.820 | −34.260 | 43.118 | 1.00 | 122.63 | N |
| ATOM | 3056 | CA | VAL | A | 342 | −63.577 | −33.475 | 44.324 | 1.00 | 116.18 | C |
| ATOM | 3057 | CB | VAL | A | 342 | −64.250 | −34.104 | 45.560 | 1.00 | 113.31 | C |
| ATOM | 3058 | CG1 | VAL | A | 342 | −63.955 | −33.282 | 46.796 | 1.00 | 101.93 | C |
| ATOM | 3059 | CG2 | VAL | A | 342 | −65.755 | −34.233 | 45.344 | 1.00 | 118.06 | C |
| ATOM | 3060 | C | VAL | A | 342 | −62.071 | −33.330 | 44.567 | 1.00 | 113.02 | C |
| ATOM | 3061 | O | VAL | A | 342 | −61.571 | −32.249 | 44.862 | 1.00 | 111.06 | O |
| ATOM | 3062 | N | VAL | A | 343 | −61.340 | −34.434 | 44.352 | 1.00 | 116.06 | N |
| ATOM | 3063 | CA | VAL | A | 343 | −59.892 | −34.416 | 44.520 | 1.00 | 115.63 | C |
| ATOM | 3064 | CB | VAL | A | 343 | −59.289 | −35.837 | 44.417 | 1.00 | 112.46 | C |
| ATOM | 3065 | CG1 | VAL | A | 343 | −57.766 | −35.789 | 44.479 | 1.00 | 101.40 | C |
| ATOM | 3066 | CG2 | VAL | A | 343 | −59.845 | −36.729 | 45.519 | 1.00 | 109.90 | C |
| ATOM | 3067 | C | VAL | A | 343 | −59.215 | −33.475 | 43.493 | 1.00 | 110.11 | C |
| ATOM | 3068 | O | VAL | A | 343 | −58.312 | −32.736 | 43.851 | 1.00 | 106.15 | O |
| ATOM | 3069 | N | PHE | A | 344 | −59.716 | −33.422 | 42.243 | 1.00 | 109.56 | N |
| ATOM | 3070 | CA | PHE | A | 344 | −59.199 | −32.443 | 41.277 | 1.00 | 104.96 | C |
| ATOM | 3071 | CB | PHE | A | 344 | −59.934 | −32.617 | 39.940 | 1.00 | 112.25 | C |
| ATOM | 3072 | CG | PHE | A | 344 | −59.560 | −31.607 | 38.888 | 1.00 | 111.01 | C |
| ATOM | 3073 | CD1 | PHE | A | 344 | −58.423 | −31.779 | 38.116 | 1.00 | 111.77 | C |
| ATOM | 3074 | CE1 | PHE | A | 344 | −58.085 | −30.858 | 37.141 | 1.00 | 113.25 | C |
| ATOM | 3075 | CZ | PHE | A | 344 | −58.893 | −29.752 | 36.921 | 1.00 | 107.90 | C |
| ATOM | 3076 | CE2 | PHE | A | 344 | −60.035 | −29.575 | 37.676 | 1.00 | 105.54 | C |
| ATOM | 3077 | CD2 | PHE | A | 344 | −60.366 | −30.501 | 38.649 | 1.00 | 108.10 | C |
| ATOM | 3078 | C | PHE | A | 344 | −59.327 | −30.987 | 41.778 | 1.00 | 101.16 | C |
| ATOM | 3079 | O | PHE | A | 344 | −58.368 | −30.239 | 41.778 | 1.00 | 102.03 | O |
| ATOM | 3080 | N | ILE | A | 345 | −60.532 | −30.586 | 42.183 | 1.00 | 99.55 | N |
| ATOM | 3081 | CA | ILE | A | 345 | −60.755 | −29.220 | 42.616 | 1.00 | 97.24 | C |
| ATOM | 3082 | CB | ILE | A | 345 | −62.261 | −28.963 | 42.858 | 1.00 | 95.46 | C |
| ATOM | 3083 | CG1 | ILE | A | 345 | −63.031 | −29.049 | 41.538 | 1.00 | 99.38 | C |
| ATOM | 3084 | CD1 | I LE | A | 345 | −64.487 | −28.666 | 41.642 | 1.00 | 95.33 | C |
| ATOM | 3085 | CG2 | ILE | A | 345 | −62.476 | −27.613 | 43.507 | 1.00 | 90.39 | C |
| ATOM | 3086 | C | ILE | A | 345 | −59.955 | −28.840 | 43.859 | 1.00 | 95.08 | C |
| ATOM | 3087 | O | ILE | A | 345 | −59.396 | −27.761 | 43.921 | 1.00 | 94.34 | O |
| ATOM | 3088 | N | TYR | A | 346 | −59.930 | −29.732 | 44.861 | 1.00 | 95.30 | N |
| ATOM | 3089 | CA | TYR | A | 346 | −59.193 | −29.486 | 46.102 | 1.00 | 91.59 | C |
| ATOM | 3090 | CB | TYR | A | 346 | −59.482 | −30.575 | 47.138 | 1.00 | 90.23 | C |
| ATOM | 3091 | CG | TYR | A | 346 | −60.858 | −30.497 | 47.754 | 1.00 | 90.08 | C |
| ATOM | 3092 | CD2 | TYR | A | 346 | −61.270 | −31.434 | 48.691 | 1.00 | 88.65 | C |
| ATOM | 3093 | CE2 | TYR | A | 346 | −62.521 | −31.367 | 49.260 | 1.00 | 97.90 | C |
| ATOM | 3094 | CZ | TYR | A | 346 | −63.384 | −30.355 | 48.891 | 1.00 | 96.65 | C |
| ATOM | 3095 | OH | TYR | A | 346 | −64.637 | −30.287 | 49.452 | 1.00 | 90.55 | O |
| ATOM | 3096 | CE1 | TYR | A | 346 | −62.998 | −29.410 | 47.968 | 1.00 | 95.40 | C |
| ATOM | 3097 | CD1 | TYR | A | 346 | −61.742 | −29.482 | 47.407 | 1.00 | 93.78 | C |
| ATOM | 3098 | C | TYR | A | 346 | −57.680 | −29.376 | 45.877 | 1.00 | 95.46 | C |
| ATOM | 3099 | O | TYR | A | 346 | −57.013 | −28.500 | 46.413 | 1.00 | 93.68 | O |
| ATOM | 3100 | N | PHE | A | 347 | −57.155 | −30.221 | 44.994 | 1.00 | 97.19 | N |
| ATOM | 3101 | CA | PHE | A | 347 | −55.773 | −30.093 | 44.553 | 1.00 | 89.20 | C |
| ATOM | 3102 | CB | PHE | A | 347 | −55.401 | −31.234 | 43.606 | 1.00 | 90.29 | C |
| ATOM | 3103 | CG | PHE | A | 347 | −53.946 | −31.606 | 43.643 | 1.00 | 87.34 | C |
| ATOM | 3104 | CD1 | PHE | A | 347 | −53.353 | −32.020 | 44.825 | 1.00 | 87.46 | C |
| ATOM | 3105 | CE1 | PHE | A | 347 | −52.017 | −32.376 | 44.862 | 1.00 | 87.47 | C |
| ATOM | 3106 | CZ | PHE | A | 347 | −51.258 | −32.328 | 43.710 | 1.00 | 86.87 | C |
| ATOM | 3107 | CE2 | PHE | A | 347 | −51.839 | −31.926 | 42.521 | 1.00 | 86.84 | C |
| ATOM | 3108 | CD2 | PHE | A | 347 | −53.177 | −31.569 | 42.490 | 1.00 | 84.96 | C |
| ATOM | 3109 | C | PHE | A | 347 | −55.501 | −28.725 | 43.897 | 1.00 | 86.45 | C |
| ATOM | 3110 | O | PHE | A | 347 | −54.549 | −28.044 | 44.224 | 1.00 | 84.24 | O |
| ATOM | 3111 | N | ASN | A | 348 | −56.393 | −28.317 | 42.992 | 1.00 | 90.08 | N |
| ATOM | 3112 | CA | ASN | A | 348 | −56.294 | −27.025 | 42.321 | 1.00 | 88.43 | C |
| ATOM | 3113 | CB | ASN | A | 348 | −57.434 | −26.856 | 41.317 | 1.00 | 99.35 | C |
| ATOM | 3114 | CG | ASN | A | 348 | −57.207 | −25.688 | 40.379 | 1.00 | 101.94 | C |
| ATOM | 3115 | OD1 | ASN | A | 348 | −56.070 | −25.401 | 39.994 | 1.00 | 97.10 | O |
| ATOM | 3116 | ND2 | ASN | A | 348 | −58.285 | −25.008 | 40.006 | 1.00 | 94.88 | N |
| ATOM | 3117 | C | ASN | A | 348 | −56.313 | −25.883 | 43.301 | 1.00 | 80.93 | C |
| ATOM | 3118 | O | ASN | A | 348 | −55.601 | −24.912 | 43.167 | 1.00 | 82.08 | O |
| ATOM | 3119 | N | ALA | A | 349 | −57.170 | −25.994 | 44.281 | 1.00 | 80.74 | N |
| ATOM | 3120 | CA | ALA | A | 349 | −57.379 | −24.912 | 45.196 | 1.00 | 82.02 | C |
| ATOM | 3121 | CB | ALA | A | 349 | −58.569 | −25.194 | 46.098 | 1.00 | 83.50 | C |
| ATOM | 3122 | C | ALA | A | 349 | −56.113 | −24.720 | 46.020 | 1.00 | 78.22 | C |
| ATOM | 3123 | O | ALA | A | 349 | −55.651 | −23.599 | 46.196 | 1.00 | 79.33 | O |
| ATOM | 3124 | N | PHE | A | 350 | −55.533 | −25.858 | 46.457 | 1.00 | 73.28 | N |
| ATOM | 3125 | CA | PHE | A | 350 | −54.277 | −25.872 | 47.192 | 1.00 | 63.02 | C |
| ATOM | 3126 | CB | PHE | A | 350 | −54.011 | −27.303 | 47.669 | 1.00 | 56.02 | C |
| ATOM | 3127 | CG | PHE | A | 350 | −52.632 | −27.527 | 48.206 | 1.00 | 58.15 | C |
| ATOM | 3128 | CD1 | PHE | A | 350 | −52.223 | −26.919 | 49.380 | 1.00 | 56.17 | C |
| ATOM | 3129 | CE1 | PHE | A | 350 | −50.957 | −27.133 | 49.879 | 1.00 | 51.76 | C |
| ATOM | 3130 | CZ | PHE | A | 350 | −50.087 | −27.976 | 49.209 | 1.00 | 56.24 | C |
| ATOM | 3131 | CE2 | PHE | A | 350 | −50.486 | −28.598 | 48.044 | 1.00 | 50.78 | C |
| ATOM | 3132 | CD2 | PHE | A | 350 | −51.753 | −28.374 | 47.551 | 1.00 | 58.56 | C |
| ATOM | 3133 | C | PHE | A | 350 | −53.055 | −25.317 | 46.406 | 1.00 | 71.11 | C |
| ATOM | 3134 | O | PHE | A | 350 | −52.267 | −24.532 | 46.916 | 1.00 | 69.47 | O |
| ATOM | 3135 | N | LEU | A | 351 | −52.874 | −25.774 | 45.169 | 1.00 | 72.91 | N |
| ATOM | 3136 | CA | LEU | A | 351 | −51.725 | −25.367 | 44.394 | 1.00 | 67.32 | C |
| ATOM | 3137 | CB | LEU | A | 351 | −51.605 | −26.188 | 43.115 | 1.00 | 67.33 | C |
| ATOM | 3138 | CG | LEU | A | 351 | −51.317 | −27.660 | 43.413 | 1.00 | 68.24 | C |
| ATOM | 3139 | CD1 | LEU | A | 351 | −51.309 | −28.464 | 42.146 | 1.00 | 73.88 | C |
| ATOM | 3140 | CD2 | LEU | A | 351 | −50.005 | −27.840 | 44.177 | 1.00 | 66.13 | C |
| ATOM | 3141 | C | LEU | A | 351 | −51.680 | −23.878 | 44.095 | 1.00 | 74.11 | C |
| ATOM | 3142 | O | LEU | A | 351 | −50.625 | −23.263 | 44.170 | 1.00 | 78.63 | O |
| ATOM | 3143 | N | GLU | A | 352 | −52.815 | −23.306 | 43.685 | 1.00 | 65.88 | N |
| ATOM | 3144 | CA | GLU | A | 352 | −52.851 | −21.900 | 43.354 | 1.00 | 70.45 | C |
| ATOM | 3145 | CB | GLU | A | 352 | −54.221 | −21.529 | 42.783 | 1.00 | 81.90 | C |
| ATOM | 3146 | CG | GLU | A | 352 | −54.625 | −22.266 | 41.526 | 1.00 | 92.70 | C |
| ATOM | 3147 | CD | GLU | A | 352 | −56.039 | −21.924 | 41.087 | 1.00 | 104.53 | C |
| ATOM | 3148 | OE1 | GLU | A | 352 | −56.931 | −21.829 | 41.961 | 1.00 | 91.96 | O |
| ATOM | 3149 | OE2 | GLU | A | 352 | −56.256 | −21.742 | 39.869 | 1.00 | 106.89 | O |
| ATOM | 3150 | C | GLU | A | 352 | −52.590 | −20.992 | 44.565 | 1.00 | 67.92 | C |
| ATOM | 3151 | O | GLU | A | 352 | −51.945 | −19.962 | 44.467 | 1.00 | 68.04 | O |
| ATOM | 3152 | N | SER | A | 353 | −53.175 | −21.373 | 45.686 | 1.00 | 62.32 | N |
| ATOM | 3153 | CA | SER | A | 353 | −53.103 | −20.639 | 46.940 | 1.00 | 60.20 | C |
| ATOM | 3154 | CB | SER | A | 353 | −54.221 | −21.095 | 47.881 | 1.00 | 56.38 | C |
| ATOM | 3155 | OG | SER | A | 353 | −54.107 | −22.472 | 48.183 | 1.00 | 61.88 | O |
| ATOM | 3156 | C | SER | A | 353 | −51.724 | −20.643 | 47.655 | 1.00 | 61.24 | C |
| ATOM | 3157 | O | SER | A | 353 | −51.295 | −19.709 | 48.378 | 1.00 | 61.80 | O |
| ATOM | 3158 | N | PHE | A | 354 | −51.084 | −21.799 | 47.509 | 1.00 | 57.97 | N |
| ATOM | 3159 | CA | PHE | A | 354 | −49.779 | −22.055 | 48.058 | 1.00 | 62.07 | C |
| ATOM | 3160 | CB | PHE | A | 354 | −49.706 | −23.449 | 48.675 | 1.00 | 61.82 | C |
| ATOM | 3161 | CG | PHE | A | 354 | −50.381 | −23.538 | 50.011 | 1.00 | 59.88 | C |
| ATOM | 3162 | CD2 | PHE | A | 354 | −49.641 | −23.497 | 51.180 | 1.00 | 58.35 | C |
| ATOM | 3163 | CE2 | PHE | A | 354 | −50.262 | −23.567 | 52.413 | 1.00 | 64.06 | C |
| ATOM | 3164 | CZ | PHE | A | 354 | −51.636 | −23.671 | 52.488 | 1.00 | 55.79 | C |
| ATOM | 3165 | CE1 | PHE | A | 354 | −52.384 | −23.708 | 51.331 | 1.00 | 52.51 | C |
| ATOM | 3166 | CD1 | PHE | A | 354 | −51.759 | −23.633 | 50.101 | 1.00 | 55.35 | C |
| ATOM | 3167 | C | PHE | A | 354 | −48.679 | −21.808 | 47.013 | 1.00 | 62.60 | C |
| ATOM | 3168 | O | PHE | A | 354 | −47.499 | −21.900 | 47.314 | 1.00 | 64.99 | O |
| ATOM | 3169 | N | GLN | A | 355 | −49.064 | −21.420 | 45.796 | 1.00 | 58.41 | N |
| ATOM | 3170 | CA | GLN | A | 355 | −48.110 | −20.956 | 44.782 | 1.00 | 54.77 | C |
| ATOM | 3171 | CB | GLN | A | 355 | −48.820 | −20.328 | 43.578 | 1.00 | 61.89 | C |
| ATOM | 3172 | CG | GLN | A | 355 | −47.864 | −19.611 | 42.621 | 1.00 | 60.83 | C |
| ATOM | 3173 | CD | GLN | A | 355 | −48.410 | −19.478 | 41.207 | 1.00 | 68.08 | C |
| ATOM | 3174 | OE1 | GLN | A | 355 | −49.060 | −20.389 | 40.690 | 1.00 | 69.83 | O |
| ATOM | 3175 | NE2 | GLN | A | 355 | −48.142 | −18.338 | 40.574 | 1.00 | 60.08 | N |
| ATOM | 3176 | C | GLN | A | 355 | −47.057 | −19.990 | 45.340 | 1.00 | 53.09 | C |
| ATOM | 3177 | O | GLN | A | 355 | −45.869 | −20.150 | 45.156 | 1.00 | 52.49 | O |
| ATOM | 3178 | N | GLY | A | 356 | −47.511 | −19.033 | 46.104 | 1.00 | 51.96 | N |
| ATOM | 3179 | CA | GLY | A | 356 | −46.646 | −18.082 | 46.728 | 1.00 | 49.35 | C |
| ATOM | 3180 | C | GLY | A | 356 | −45.723 | −18.651 | 47.747 | 1.00 | 52.51 | C |
| ATOM | 3181 | O | GLY | A | 356 | −44.598 | −18.233 | 47.860 | 1.00 | 53.52 | O |
| ATOM | 3182 | N | PHE | A | 357 | −46.191 | −19.603 | 48.524 | 1.00 | 51.73 | N |
| ATOM | 3183 | CA | PHE | A | 357 | −45.381 | −20.207 | 49.558 | 1.00 | 46.13 | C |
| ATOM | 3184 | CB | PHE | A | 357 | −46.258 | −21.164 | 50.377 | 1.00 | 45.66 | C |
| ATOM | 3185 | CG | PHE | A | 357 | −45.523 | −21.912 | 51.455 | 1.00 | 39.68 | C |
| ATOM | 3186 | CD1 | PHE | A | 357 | −45.232 | −21.307 | 52.667 | 1.00 | 38.21 | C |
| ATOM | 3187 | CE1 | PHE | A | 357 | −44.563 | −22.000 | 53.664 | 1.00 | 36.62 | C |
| ATOM | 3188 | CZ | PHE | A | 357 | −44.190 | −23.313 | 53.456 | 1.00 | 34.39 | C |
| ATOM | 3189 | CE2 | PHE | A | 357 | −44.484 | −23.932 | 52.255 | 1.00 | 33.13 | C |
| ATOM | 3190 | CD2 | PHE | A | 357 | −45.151 | −23.234 | 51.265 | 1.00 | 36.74 | C |
| ATOM | 3191 | C | PHE | A | 357 | −44.176 | −20.951 | 48.942 | 1.00 | 44.25 | C |
| ATOM | 3192 | O | PHE | A | 357 | −43.051 | −20.851 | 49.395 | 1.00 | 45.02 | O |
| ATOM | 3193 | N | PHE | A | 358 | −44.428 | −21.668 | 47.856 | 1.00 | 44.40 | N |
| ATOM | 3194 | CA | PHE | A | 358 | −43.385 | −22.389 | 47.155 | 1.00 | 48.86 | C |
| ATOM | 3195 | CB | PHE | A | 358 | −43.956 | −23.158 | 45.962 | 1.00 | 47.26 | C |
| ATOM | 3196 | CG | PHE | A | 358 | −44.988 | −24.176 | 46.338 | 1.00 | 51.38 | C |
| ATOM | 3197 | CD1 | PHE | A | 358 | −44.960 | −24.790 | 47.579 | 1.00 | 51.40 | C |
| ATOM | 3198 | CE1 | PHE | A | 358 | −45.916 | −25.727 | 47.922 | 1.00 | 59.21 | C |
| ATOM | 3199 | CZ | PHE | A | 358 | −46.914 | −26.059 | 47.024 | 1.00 | 62.21 | C |
| ATOM | 3200 | CE2 | PHE | A | 358 | −46.954 | −25.453 | 45.787 | 1.00 | 60.32 | C |
| ATOM | 3201 | CD2 | PHE | A | 358 | −45.994 | −24.517 | 45.451 | 1.00 | 61.25 | C |
| ATOM | 3202 | C | PHE | A | 358 | −42.295 | −21.460 | 46.677 | 1.00 | 48.91 | C |
| ATOM | 3203 | O | PHE | A | 358 | −41.125 | −21.714 | 46.896 | 1.00 | 55.72 | O |
| ATOM | 3204 | N | VAL | A | 359 | −42.687 | −20.330 | 46.109 | 1.00 | 48.79 | N |
| ATOM | 3205 | CA | VAL | A | 359 | −41.728 | −19.297 | 45.739 | 1.00 | 49.20 | C |
| ATOM | 3206 | CB | VAL | A | 359 | −42.406 | −18.117 | 44.992 | 1.00 | 46.87 | C |
| ATOM | 3207 | CG1 | VAL | A | 359 | −41.383 | −17.054 | 44.607 | 1.00 | 43.36 | C |
| ATOM | 3208 | CG2 | VAL | A | 359 | −43.131 | −18.621 | 43.756 | 1.00 | 37.11 | C |
| ATOM | 3209 | C | VAL | A | 359 | −40.913 | −18.775 | 46.945 | 1.00 | 44.71 | C |
| ATOM | 3210 | O | VAL | A | 359 | −39.735 | −18.527 | 46.828 | 1.00 | 46.70 | O |
| ATOM | 3211 | N | SER | A | 360 | −41.532 | −18.584 | 48.100 | 1.00 | 42.23 | N |
| ATOM | 3212 | CA | SER | A | 360 | −40.797 | −18.104 | 49.263 | 1.00 | 46.24 | C |
| ATOM | 3213 | CB | SER | A | 360 | −41.740 | −17.707 | 50.401 | 1.00 | 47.19 | C |
| ATOM | 3214 | OG | SER | A | 360 | −42.490 | −18.817 | 50.849 | 1.00 | 59.96 | O |
| ATOM | 3215 | C | SER | A | 360 | −39.733 | −19.062 | 49.744 | 1.00 | 48.65 | C |
| ATOM | 3216 | O | SER | A | 360 | −38.599 | −18.699 | 49.984 | 1.00 | 51.02 | O |
| ATOM | 3217 | N | VAL | A | 361 | −40.107 | −20.318 | 49.829 | 1.00 | 45.19 | N |
| ATOM | 3218 | CA | VAL | A | 361 | −39.177 | −21.368 | 50.151 | 1.00 | 47.68 | C |
| ATOM | 3219 | CB | VAL | A | 361 | −39.914 | −22.723 | 50.201 | 1.00 | 43.50 | C |
| ATOM | 3220 | CG1 | VAL | A | 361 | −38.934 | −23.881 | 50.306 | 1.00 | 49.64 | C |
| ATOM | 3221 | CG2 | VAL | A | 361 | −40.892 | −22.738 | 51.357 | 1.00 | 41.13 | C |
| ATOM | 3222 | C | VAL | A | 361 | −38.039 | −21.455 | 49.145 | 1.00 | 56.02 | C |
| ATOM | 3223 | O | VAL | A | 361 | −36.883 | −21.611 | 49.489 | 1.00 | 60.09 | O |
| ATOM | 3224 | N | PHE | A | 362 | −38.395 | −21.415 | 47.880 | 1.00 | 53.60 | N |
| ATOM | 3225 | CA | PHE | A | 362 | −37.440 | −21.555 | 46.802 | 1.00 | 52.73 | C |
| ATOM | 3226 | CB | PHE | A | 362 | −38.242 | −21.572 | 45.490 | 1.00 | 49.77 | C |
| ATOM | 3227 | CG | PHE | A | 362 | −37.435 | −21.352 | 44.240 | 1.00 | 49.53 | C |
| ATOM | 3228 | CD1 | PHE | A | 362 | −36.650 | −22.363 | 43.708 | 1.00 | 55.83 | C |
| ATOM | 3229 | CE1 | PHE | A | 362 | −35.937 | −22.163 | 42.535 | 1.00 | 50.44 | C |
| ATOM | 3230 | CZ | PHE | A | 362 | −36.027 | −20.954 | 41.872 | 1.00 | 52.27 | C |
| ATOM | 3231 | CE2 | PHE | A | 362 | −36.822 | −19.946 | 42.383 | 1.00 | 48.35 | C |
| ATOM | 3232 | CD2 | PHE | A | 362 | −37.528 | −20.152 | 43.553 | 1.00 | 49.89 | C |
| ATOM | 3233 | C | PHE | A | 362 | −36.376 | −20.477 | 46.775 | 1.00 | 51.94 | C |
| ATOM | 3234 | O | PHE | A | 362 | −35.207 | −20.766 | 46.637 | 1.00 | 51.90 | O |
| ATOM | 3235 | N | ALA | A | 363 | −36.799 | −19.229 | 46.878 | 1.00 | 51.13 | N |
| ATOM | 3236 | CA | ALA | A | 363 | −35.880 | −18.116 | 46.891 | 1.00 | 52.99 | C |
| ATOM | 3237 | CB | ALA | A | 363 | −36.644 | −16.798 | 46.931 | 1.00 | 50.09 | C |
| ATOM | 3238 | C | ALA | A | 363 | −34.930 | −18.203 | 48.048 | 1.00 | 57.16 | C |
| ATOM | 3239 | O | ALA | A | 363 | −33.738 | −18.044 | 47.891 | 1.00 | 65.20 | O |
| ATOM | 3240 | N | CYS | A | 364 | −35.470 | −18.530 | 49.214 | 1.00 | 56.79 | N |
| ATOM | 3241 | CA | CYS | A | 364 | −34.665 | −18.767 | 50.398 | 1.00 | 63.58 | C |
| ATOM | 3242 | CB | CYS | A | 364 | −35.553 | −19.010 | 51.622 | 1.00 | 66.47 | C |
| ATOM | 3243 | SG | CYS | A | 364 | −36.467 | −17.546 | 52.175 | 1.00 | 67.80 | S |
| ATOM | 3244 | C | CYS | A | 364 | −33.665 | −19.899 | 50.231 | 1.00 | 66.28 | C |
| ATOM | 3245 | O | CYS | A | 364 | −32.540 | −19.771 | 50.658 | 1.00 | 65.26 | O |
| ATOM | 3246 | N | PHE | A | 365 | −34.074 | −21.002 | 49.607 | 1.00 | 61.40 | N |
| ATOM | 3247 | CA | PHE | A | 365 | −33.165 | −22.121 | 49.357 | 1.00 | 61.42 | C |
| ATOM | 3248 | CB | PHE | A | 365 | −33.912 | −23.289 | 48.714 | 1.00 | 56.05 | C |
| ATOM | 3249 | CG | PHE | A | 365 | −33.042 | −24.477 | 48.414 | 1.00 | 63.71 | C |
| ATOM | 3250 | CD1 | PHE | A | 365 | −32.561 | −25.276 | 49.439 | 1.00 | 64.92 | C |
| ATOM | 3251 | CE1 | PHE | A | 365 | −31.765 | −26.373 | 49.164 | 1.00 | 70.51 | C |
| ATOM | 3252 | CZ | PHE | A | 365 | −31.447 | −26.688 | 47.854 | 1.00 | 74.16 | C |
| ATOM | 3253 | CE2 | PHE | A | 365 | −31.924 | −25.902 | 46.823 | 1.00 | 66.06 | C |
| ATOM | 3254 | CD2 | PHE | A | 365 | −32.718 | −24.804 | 47.105 | 1.00 | 65.47 | C |
| ATOM | 3255 | C | PHE | A | 365 | −31.948 | −21.707 | 48.495 | 1.00 | 63.26 | C |
| ATOM | 3256 | O | PHE | A | 365 | −30.810 | −22.013 | 48.803 | 1.00 | 70.15 | O |
| ATOM | 3257 | N | LEU | A | 366 | −32.191 | −20.958 | 47.434 | 1.00 | 59.91 | N |
| ATOM | 3258 | CA | LEU | A | 366 | −31.095 | −20.433 | 46.624 | 1.00 | 57.81 | C |
| ATOM | 3259 | CB | LEU | A | 366 | −31.629 | −19.740 | 45.370 | 1.00 | 56.21 | C |
| ATOM | 3260 | CG | LEU | A | 366 | −32.467 | −20.658 | 44.474 | 1.00 | 52.76 | C |
| ATOM | 3261 | CD1 | LEU | A | 366 | −32.883 | −19.948 | 43.201 | 1.00 | 51.99 | C |
| ATOM | 3262 | CD2 | LEU | A | 366 | −31.709 | −21.934 | 44.153 | 1.00 | 48.61 | C |
| ATOM | 3263 | C | LEU | A | 366 | −30.150 | −19.514 | 47.421 | 1.00 | 73.10 | C |
| ATOM | 3264 | O | LEU | A | 366 | −28.940 | −19.651 | 47.388 | 1.00 | 78.06 | O |
| ATOM | 3265 | N | ASN | A | 367 | −30.719 | −18.618 | 48.207 | 1.00 | 74.85 | N |
| ATOM | 3266 | CA | ASN | A | 367 | −29.920 | −17.732 | 49.041 | 1.00 | 73.51 | C |
| ATOM | 3267 | CB | ASN | A | 367 | −30.795 | −16.670 | 49.693 | 1.00 | 70.08 | C |
| ATOM | 3268 | CG | ASN | A | 367 | −29.981 | −15.593 | 50.367 | 1.00 | 84.83 | C |
| ATOM | 3269 | OD1 | ASN | A | 367 | −28.849 | −15.313 | 49.963 | 1.00 | 82.55 | O |
| ATOM | 3270 | ND2 | ASN | A | 367 | −30.546 | −14.984 | 51.404 | 1.00 | 89.37 | N |
| ATOM | 3271 | C | ASN | A | 367 | −29.120 | −18.498 | 50.131 | 1.00 | 84.32 | C |
| ATOM | 3272 | O | ASN | A | 367 | −27.939 | −18.242 | 50.340 | 1.00 | 90.18 | O |
| ATOM | 3273 | N | SER | A | 368 | −29.813 | −19.450 | 50.783 | 1.00 | 82.08 | N |
| ATOM | 3274 | CA | SER | A | 368 | −29.308 | −20.345 | 51.848 | 1.00 | 83.76 | C |
| ATOM | 3275 | CB | SER | A | 368 | −28.517 | −21.509 | 51.228 | 1.00 | 80.60 | C |
| ATOM | 3276 | OG | SER | A | 368 | −27.292 | −21.065 | 50.669 | 1.00 | 81.24 | O |
| ATOM | 3277 | C | SER | A | 368 | −28.525 | −19.800 | 53.061 | 1.00 | 90.19 | C |
| ATOM | 3278 | O | SER | A | 368 | −28.906 | −20.188 | 54.202 | 1.00 | 94.97 | O |
| ATOM | 3279 | C24 | CP3 | A | 900 | −45.009 | −18.430 | 37.839 | 1.00 | 51.17 | C |
| ATOM | 3280 | C23 | CP3 | A | 900 | −45.855 | −19.271 | 36.915 | 1.00 | 46.63 | C |
| ATOM | 3281 | C20 | CP3 | A | 900 | −44.996 | −19.957 | 35.878 | 1.00 | 51.19 | C |
| ATOM | 3282 | C21 | CP3 | A | 900 | −45.795 | −21.066 | 35.229 | 1.00 | 55.05 | C |
| ATOM | 3283 | C22 | CP3 | A | 900 | −44.886 | −22.010 | 34.488 | 1.00 | 58.39 | C |
| ATOM | 3284 | N19 | CP3 | A | 900 | −43.811 | −20.471 | 36.518 | 1.00 | 55.64 | N |
| ATOM | 3285 | C15 | CP3 | A | 900 | −42.621 | −20.356 | 35.903 | 1.00 | 59.64 | C |
| ATOM | 3286 | C14 | CP3 | A | 900 | −42.401 | −19.406 | 34.927 | 1.00 | 55.78 | C |
| ATOM | 3287 | C13 | CP3 | A | 900 | −41.176 | −19.302 | 34.303 | 1.00 | 48.97 | C |
| ATOM | 3288 | C18 | CP3 | A | 900 | −41.002 | −18.252 | 33.256 | 1.00 | 42.92 | C |
| ATOM | 3289 | N12 | CP3 | A | 900 | −40.142 | −20.103 | 34.600 | 1.00 | 46.63 | N |
| ATOM | 3290 | C16 | CP3 | A | 900 | −41.511 | −21.245 | 36.246 | 1.00 | 57.38 | C |
| ATOM | 3291 | C17 | CP3 | A | 900 | −41.670 | −22.305 | 37.296 | 1.00 | 54.75 | C |
| ATOM | 3292 | C11 | CP3 | A | 900 | −40.245 | −21.054 | 35.527 | 1.00 | 48.78 | C |
| ATOM | 3293 | O10 | CP3 | A | 900 | −39.201 | −21.855 | 35.817 | 1.00 | 45.98 | O |
| ATOM | 3294 | C1 | CP3 | A | 900 | −37.999 | −21.731 | 35.204 | 1.00 | 53.06 | C |
| ATOM | 3295 | C6 | CP3 | A | 900 | −37.752 | −22.233 | 33.840 | 1.00 | 57.37 | C |
| ATOM | 3296 | C8 | CP3 | A | 900 | −38.858 | −22.894 | 33.075 | 1.00 | 50.92 | C |
| ATOM | 3297 | C5 | CP3 | A | 900 | −36.493 | −22.079 | 33.286 | 1.00 | 57.60 | C |
| ATOM | 3298 | C4 | CP3 | A | 900 | −35.468 | −21.461 | 34.003 | 1.00 | 56.51 | C |
| ATOM | 3299 | C7 | CP3 | A | 900 | −34.109 | −21.301 | 33.386 | 1.00 | 54.11 | C |
| ATOM | 3300 | C3 | CP3 | A | 900 | −35.653 | −20.979 | 35.288 | 1.00 | 53.25 | C |
| ATOM | 3301 | C2 | CP3 | A | 900 | −36.879 | −21.095 | 35.911 | 1.00 | 53.15 | C |
| ATOM | 3302 | C9 | CP3 | A | 900 | −37.085 | −20.579 | 37.295 | 1.00 | 48.88 | C |
| ATOM | 3303 | O1 | POP | A | 901 | −35.411 | −4.561 | 46.553 | 1.00 | 58.03 | O |
| ATOM | 3304 | C2 | POP | A | 901 | −36.091 | −4.106 | 45.646 | 1.00 | 66.92 | C |
| ATOM | 3305 | C3 | POP | A | 901 | −37.477 | −4.631 | 45.370 | 1.00 | 58.71 | C |
| ATOM | 3306 | C4 | POP | A | 901 | −38.501 | −3.638 | 45.900 | 1.00 | 57.15 | C |
| ATOM | 3307 | C5 | POP | A | 901 | −39.487 | −3.213 | 44.822 | 1.00 | 49.53 | C |
| ATOM | 3308 | C6 | POP | A | 901 | −40.866 | −3.770 | 45.121 | 1.00 | 59.59 | C |
| ATOM | 3309 | C7 | POP | A | 901 | −41.823 | −2.686 | 45.590 | 1.00 | 62.13 | C |
| ATOM | 3310 | C8 | POP | A | 901 | −43.242 | −3.222 | 45.744 | 1.00 | 59.53 | C |
| ATOM | 3311 | C9 | POP | A | 901 | −43.711 | −3.050 | 47.186 | 1.00 | 57.50 | C |
| ATOM | 3312 | C10 | POP | A | 901 | −44.157 | −4.379 | 47.779 | 1.00 | 55.88 | C |
| ATOM | 3313 | C11 | POP | A | 901 | −43.281 | −4.877 | 48.929 | 1.00 | 65.27 | C |
| ATOM | 3314 | C12 | POP | A | 901 | −44.045 | −4.808 | 50.251 | 1.00 | 62.75 | C |
| ATOM | 3315 | C13 | POP | A | 901 | −43.234 | −5.259 | 51.471 | 1.00 | 55.24 | C |
| ATOM | 3316 | C14 | POP | A | 901 | −43.336 | −4.232 | 52.597 | 1.00 | 55.42 | C |
| ATOM | 3317 | C15 | POP | A | 901 | −42.084 | −3.412 | 52.892 | 1.00 | 68.76 | C |
| ATOM | 3318 | C16 | POP | A | 901 | −41.214 | −4.121 | 53.937 | 1.00 | 68.02 | C |
| ATOM | 3319 | C17 | POP | A | 901 | −41.165 | −3.344 | 55.235 | 1.00 | 58.81 | C |
| ATOM | 3320 | O | POP | A | 901 | −35.485 | −3.189 | 44.695 | 1.00 | 82.34 | O |
| ATOM | 3321 | C1 | POP | A | 901 | −34.675 | −3.683 | 43.632 | 1.00 | 70.92 | C |
| ATOM | 3322 | C | POP | A | 901 | −34.104 | −2.489 | 42.868 | 1.00 | 72.59 | C |
| ATOM | 3323 | C18 | POP | A | 901 | −32.685 | −2.314 | 43.368 | 1.00 | 73.18 | C |
| ATOM | 3324 | O2 | POP | A | 901 | −32.101 | −1.320 | 42.538 | 1.00 | 86.11 | O |
| ATOM | 3325 | P | POP | A | 901 | −30.519 | −1.291 | 42.289 | 1.00 | 106.64 | P |
| ATOM | 3326 | O5 | POP | A | 901 | −30.219 | 0.285 | 42.350 | 1.00 | 106.12 | O |
| ATOM | 3327 | C19 | POP | A | 901 | −28.966 | 0.758 | 42.835 | 1.00 | 95.85 | C |
| ATOM | 3328 | C20 | POP | A | 901 | −28.537 | 1.965 | 42.010 | 1.00 | 98.89 | C |
| ATOM | 3329 | O6 | POP | A | 901 | −28.697 | 1.948 | 40.585 | 1.00 | 92.33 | O |
| ATOM | 3330 | C21 | POP | A | 901 | −27.931 | 3.183 | 42.702 | 1.00 | 105.88 | C |
| ATOM | 3331 | O7 | POP | A | 901 | −27.852 | 4.291 | 41.795 | 1.00 | 105.03 | O |
| ATOM | 3332 | O3 | POP | A | 901 | −29.703 | −2.085 | 43.422 | 1.00 | 93.91 | O |
| ATOM | 3333 | O4 | POP | A | 901 | −30.195 | −1.813 | 40.945 | 1.00 | 91.12 | O |
| ATOM | 3334 | O8 | POP | A | 901 | −34.791 | −1.293 | 43.228 | 1.00 | 84.87 | O |
| ATOM | 3335 | C22 | POP | A | 901 | −35.685 | −0.769 | 42.205 | 1.00 | 73.30 | C |
| ATOM | 3336 | O9 | POP | A | 901 | −35.442 | 0.347 | 41.784 | 1.00 | 80.69 | O |
| ATOM | 3337 | C23 | POP | A | 901 | −36.928 | −1.477 | 41.704 | 1.00 | 57.96 | C |
| ATOM | 3338 | C24 | POP | A | 901 | −37.867 | −0.441 | 41.079 | 1.00 | 60.83 | C |
| ATOM | 3339 | C25 | POP | A | 901 | −39.319 | −0.422 | 41.598 | 1.00 | 68.14 | C |
| ATOM | 3340 | C26 | POP | A | 901 | −40.289 | −0.701 | 40.442 | 1.00 | 67.23 | C |
| ATOM | 3341 | C27 | POP | A | 901 | −41.700 | −0.133 | 40.347 | 1.00 | 65.91 | C |
| ATOM | 3342 | C28 | POP | A | 901 | −42.721 | −1.095 | 40.989 | 1.00 | 66.97 | C |
| ATOM | 3343 | C29 | POP | A | 901 | −43.890 | −1.150 | 40.018 | 1.00 | 70.10 | C |
| ATOM | 3344 | C30 | POP | A | 901 | −45.129 | −1.613 | 40.727 | 1.00 | 64.67 | C |
| ATOM | 3345 | C31 | POP | A | 901 | −46.322 | −1.251 | 40.278 | 1.00 | 61.34 | C |
| ATOM | 3346 | C32 | POP | A | 901 | −46.378 | −0.204 | 39.197 | 1.00 | 66.04 | C |
| ATOM | 3347 | C33 | POP | A | 901 | −47.319 | −0.618 | 38.061 | 1.00 | 59.09 | C |
| ATOM | 3348 | C34 | POP | A | 901 | −48.791 | −0.441 | 38.435 | 1.00 | 61.14 | C |
| ATOM | 3349 | C35 | POP | A | 901 | −49.705 | −0.470 | 37.210 | 1.00 | 56.57 | C |
| ATOM | 3350 | C36 | POP | A | 901 | −51.125 | −0.874 | 37.591 | 1.00 | 47.32 | C |
| ATOM | 3351 | C37 | POP | A | 901 | −52.195 | −0.391 | 36.623 | 1.00 | 49.40 | C |
| ATOM | 3352 | C38 | POP | A | 901 | −52.023 | −1.052 | 35.263 | 1.00 | 45.26 | C |
| ATOM | 3353 | C39 | POP | A | 901 | −52.964 | −0.484 | 34.220 | 1.00 | 26.81 | C |
| ATOM | 3354 | O1 | POP | A | 902 | −53.504 | 20.713 | 25.159 | 1.00 | 37.72 | O |
| ATOM | 3355 | C2 | POP | A | 902 | −52.695 | 19.798 | 25.129 | 1.00 | 55.67 | C |
| ATOM | 3356 | C3 | POP | A | 902 | −51.243 | 20.043 | 24.807 | 1.00 | 44.70 | C |
| ATOM | 3357 | C4 | POP | A | 902 | −50.404 | 19.734 | 26.038 | 1.00 | 46.36 | C |
| ATOM | 3358 | C5 | POP | A | 902 | −49.384 | 18.641 | 25.759 | 1.00 | 51.73 | C |
| ATOM | 3359 | C6 | POP | A | 902 | −47.979 | 19.212 | 25.803 | 1.00 | 55.55 | C |
| ATOM | 3360 | C7 | POP | A | 902 | −47.074 | 18.400 | 26.714 | 1.00 | 50.97 | C |
| ATOM | 3361 | C8 | POP | A | 902 | −45.720 | 19.077 | 26.902 | 1.00 | 63.12 | C |
| ATOM | 3362 | C9 | POP | A | 902 | −45.502 | 20.107 | 25.797 | 1.00 | 62.66 | C |
| ATOM | 3363 | C10 | POP | A | 902 | −44.982 | 21.422 | 26.357 | 1.00 | 54.50 | C |
| ATOM | 3364 | C11 | POP | A | 902 | −45.207 | 22.606 | 25.418 | 1.00 | 45.78 | C |
| ATOM | 3365 | C12 | POP | A | 902 | −44.403 | 23.826 | 25.866 | 1.00 | 45.86 | C |
| ATOM | 3366 | C13 | POP | A | 902 | −44.758 | 25.101 | 25.094 | 1.00 | 53.33 | C |
| ATOM | 3367 | C14 | POP | A | 902 | −45.271 | 26.194 | 26.030 | 1.00 | 52.68 | C |
| ATOM | 3368 | C15 | POP | A | 902 | −45.409 | 27.587 | 25.423 | 1.00 | 53.25 | C |
| ATOM | 3369 | C16 | POP | A | 902 | −46.271 | 28.492 | 26.311 | 1.00 | 58.88 | C |
| ATOM | 3370 | C17 | POP | A | 902 | −45.813 | 29.932 | 26.241 | 1.00 | 67.76 | C |
| ATOM | 3371 | O | POP | A | 902 | −53.152 | 18.429 | 25.295 | 1.00 | 64.49 | O |
| ATOM | 3372 | C1 | POP | A | 902 | −54.301 | 18.124 | 26.079 | 1.00 | 61.01 | C |
| ATOM | 3373 | C | POP | A | 902 | −54.322 | 16.609 | 26.276 | 1.00 | 57.12 | C |
| ATOM | 3374 | C18 | POP | A | 902 | −55.739 | 16.152 | 26.011 | 1.00 | 68.08 | C |
| ATOM | 3375 | O2 | POP | A | 902 | −56.542 | 16.832 | 26.967 | 1.00 | 77.30 | O |
| ATOM | 3376 | P | POP | A | 902 | −57.891 | 16.173 | 27.523 | 1.00 | 112.28 | P |
| ATOM | 3377 | O5 | POP | A | 902 | −57.579 | 16.108 | 29.097 | 1.00 | 107.81 | O |
| ATOM | 3378 | C19 | POP | A | 902 | −58.618 | 16.344 | 30.041 | 1.00 | 98.35 | C |
| ATOM | 3379 | C20 | POP | A | 902 | −58.100 | 16.064 | 31.446 | 1.00 | 105.01 | C |
| ATOM | 3380 | O6 | POP | A | 902 | −57.319 | 17.052 | 32.132 | 1.00 | 112.39 | O |
| ATOM | 3381 | C21 | POP | A | 902 | −58.423 | 14.734 | 32.120 | 1.00 | 102.42 | C |
| ATOM | 3382 | O7 | POP | A | 902 | −59.524 | 14.882 | 33.025 | 1.00 | 91.58 | O |
| ATOM | 3383 | O3 | POP | A | 902 | −59.182 | 17.081 | 27.227 | 1.00 | 93.29 | O |
| ATOM | 3384 | O4 | POP | A | 902 | −58.069 | 14.812 | 26.975 | 1.00 | 81.69 | O |
| ATOM | 3385 | O8 | POP | A | 902 | −54.083 | 16.309 | 27.645 | 1.00 | 66.66 | O |
| ATOM | 3386 | C22 | POP | A | 902 | −52.838 | 15.595 | 27.883 | 1.00 | 72.52 | C |
| ATOM | 3387 | O9 | POP | A | 902 | −52.884 | 14.649 | 28.648 | 1.00 | 80.03 | O |
| ATOM | 3388 | C23 | POP | A | 902 | −51.526 | 15.922 | 27.202 | 1.00 | 53.48 | C |
| ATOM | 3389 | C24 | POP | A | 902 | −50.381 | 15.418 | 28.082 | 1.00 | 41.40 | C |
| ATOM | 3390 | C25 | POP | A | 902 | −49.451 | 14.367 | 27.447 | 1.00 | 47.58 | C |
| ATOM | 3391 | C26 | POP | A | 902 | −48.112 | 14.360 | 28.194 | 1.00 | 62.75 | C |
| ATOM | 3392 | C27 | POP | A | 902 | −46.877 | 13.593 | 27.740 | 1.00 | 61.29 | C |
| ATOM | 3393 | C28 | POP | A | 902 | −45.628 | 14.131 | 28.466 | 1.00 | 61.67 | C |
| ATOM | 3394 | C29 | POP | A | 902 | −44.741 | 14.716 | 27.379 | 1.00 | 52.27 | C |
| ATOM | 3395 | C30 | POP | A | 902 | −43.339 | 14.841 | 27.894 | 1.00 | 51.01 | C |
| ATOM | 3396 | C31 | POP | A | 902 | −42.335 | 14.384 | 27.164 | 1.00 | 53.44 | C |
| ATOM | 3397 | C32 | POP | A | 902 | −41.000 | 14.208 | 27.837 | 1.00 | 67.25 | C |
| ATOM | 3398 | C33 | POP | A | 902 | −40.498 | 12.772 | 27.656 | 1.00 | 72.82 | C |
| ATOM | 3399 | C34 | POP | A | 902 | −38.993 | 12.646 | 27.894 | 1.00 | 56.50 | C |
| ATOM | 3400 | C35 | POP | A | 902 | −38.536 | 11.190 | 27.836 | 1.00 | 45.88 | C |
| ATOM | 3401 | C36 | POP | A | 902 | −37.040 | 11.081 | 27.570 | 1.00 | 34.71 | C |
| ATOM | 3402 | C37 | POP | A | 902 | −36.504 | 9.664 | 27.711 | 1.00 | 37.29 | C |
| ATOM | 3403 | C38 | POP | A | 902 | −34.983 | 9.671 | 27.705 | 1.00 | 35.78 | C |
| ATOM | 3404 | C39 | POP | A | 902 | −34.403 | 8.273 | 27.671 | 1.00 | 34.18 | C |
| ATOM | 3405 | O | MOO | A | 903 | −32.964 | −20.594 | 54.113 | 1.00 | 84.30 | O |
| ATOM | 3406 | C17 | MOO | A | 903 | −33.905 | −20.270 | 54.819 | 1.00 | 86.56 | C |
| ATOM | 3407 | O1 | MOO | A | 903 | −34.284 | −18.867 | 54.908 | 1.00 | 88.49 | O |
| ATOM | 3408 | C18 | MOO | A | 903 | −33.408 | −17.895 | 55.479 | 1.00 | 65.99 | C |
| ATOM | 3409 | C19 | MOO | A | 903 | −32.376 | −17.437 | 54.456 | 1.00 | 70.94 | C |
| ATOM | 3410 | C20 | MOO | A | 903 | −31.800 | −16.096 | 54.896 | 1.00 | 80.64 | C |
| ATOM | 3411 | O3 | MOO | A | 903 | −31.017 | −15.537 | 53.836 | 1.00 | 88.63 | O |
| ATOM | 3412 | O2 | MOO | A | 903 | −33.003 | −17.279 | 53.179 | 1.00 | 77.48 | O |
| ATOM | 3413 | C16 | MOO | A | 903 | −34.669 | −21.312 | 55.602 | 1.00 | 79.21 | C |
| ATOM | 3414 | C15 | MOO | A | 903 | −35.941 | −20.693 | 56.168 | 1.00 | 71.49 | C |
| ATOM | 3415 | C14 | MOO | A | 903 | −37.015 | −20.547 | 55.095 | 1.00 | 64.34 | C |
| ATOM | 3416 | C13 | MOO | A | 903 | −38.124 | −19.623 | 55.579 | 1.00 | 54.03 | C |
| ATOM | 3417 | C12 | MOO | A | 903 | −39.316 | −19.633 | 54.630 | 1.00 | 53.30 | C |
| ATOM | 3418 | C11 | MOO | A | 903 | −40.375 | −18.649 | 55.112 | 1.00 | 60.09 | C |
| ATOM | 3419 | C10 | MOO | A | 903 | −40.734 | −18.914 | 56.571 | 1.00 | 61.90 | C |
| ATOM | 3420 | C9 | MOO | A | 903 | −42.083 | −19.587 | 56.651 | 1.00 | 54.41 | C |
| ATOM | 3421 | C8 | MOO | A | 903 | −43.180 | −18.900 | 56.347 | 1.00 | 46.31 | C |
| ATOM | 3422 | C7 | MOO | A | 903 | −44.537 | −19.556 | 56.421 | 1.00 | 48.55 | C |
| ATOM | 3423 | C6 | MOO | A | 903 | −44.780 | −20.093 | 57.826 | 1.00 | 53.94 | C |
| ATOM | 3424 | C5 | MOO | A | 903 | −46.223 | −20.559 | 57.974 | 1.00 | 52.72 | C |
| ATOM | 3425 | C4 | MOO | A | 903 | −47.181 | −19.372 | 57.991 | 1.00 | 49.77 | C |
| ATOM | 3426 | C3 | MOO | A | 903 | −47.553 | −18.998 | 59.421 | 1.00 | 52.37 | C |
| ATOM | 3427 | C2 | MOO | A | 903 | −48.447 | −20.066 | 60.042 | 1.00 | 50.66 | C |
| ATOM | 3428 | C1 | MOO | A | 903 | −48.111 | −20.278 | 61.513 | 1.00 | 55.40 | C |
| ATOM | 3429 | C | MOO | A | 903 | −46.722 | −20.858 | 61.676 | 1.00 | 51.97 | C |
| ATOM | 3430 | O | MOO | A | 904 | −39.461 | 1.406 | 36.380 | 1.00 | 79.87 | O |
| ATOM | 3431 | C17 | MOO | A | 904 | −39.702 | 0.509 | 35.588 | 1.00 | 71.10 | C |
| ATOM | 3432 | O1 | MOO | A | 904 | −38.659 | 0.066 | 34.673 | 1.00 | 63.88 | O |
| ATOM | 3433 | C18 | MOO | A | 904 | −38.376 | 0.796 | 33.479 | 1.00 | 68.53 | C |
| ATOM | 3434 | C19 | MOO | A | 904 | −37.728 | 2.136 | 33.808 | 1.00 | 78.04 | C |
| ATOM | 3435 | C20 | MOO | A | 904 | −36.212 | 1.973 | 33.860 | 1.00 | 87.23 | C |
| ATOM | 3436 | O3 | MOO | A | 904 | −35.720 | 1.602 | 32.566 | 1.00 | 82.87 | O |
| ATOM | 3437 | O2 | MOO | A | 904 | −38.069 | 3.087 | 32.793 | 1.00 | 71.50 | O |
| ATOM | 3438 | C16 | MOO | A | 904 | −41.065 | −0.143 | 35.557 | 1.00 | 65.21 | C |
| ATOM | 3439 | C15 | MOO | A | 904 | −42.149 | 0.880 | 35.229 | 1.00 | 61.26 | C |
| ATOM | 3440 | C14 | MOO | A | 904 | −42.676 | 0.696 | 33.809 | 1.00 | 55.19 | C |
| ATOM | 3441 | C13 | MOO | A | 904 | −41.824 | 1.456 | 32.798 | 1.00 | 64.72 | C |
| ATOM | 3442 | C12 | MOO | A | 904 | −41.917 | 0.835 | 31.408 | 1.00 | 53.61 | C |
| ATOM | 3443 | C11 | MOO | A | 904 | −40.980 | 1.541 | 30.434 | 1.00 | 41.04 | C |
| ATOM | 3444 | C10 | MOO | A | 904 | −40.093 | 0.548 | 29.691 | 1.00 | 37.56 | C |
| ATOM | 3445 | C9 | MOO | A | 904 | −40.952 | −0.444 | 28.943 | 1.00 | 39.69 | C |
| ATOM | 3446 | C8 | MOO | A | 904 | −40.405 | −1.354 | 28.140 | 1.00 | 40.80 | C |
| ATOM | 3447 | C7 | MOO | A | 904 | −38.908 | −1.424 | 27.944 | 1.00 | 42.09 | C |
| ATOM | 3448 | C6 | MOO | A | 904 | −38.576 | −2.587 | 27.015 | 1.00 | 37.60 | C |
| ATOM | 3449 | C5 | MOO | A | 904 | −37.090 | −2.630 | 26.677 | 1.00 | 45.99 | C |
| ATOM | 3450 | C4 | MOO | A | 904 | −36.771 | −3.812 | 25.768 | 1.00 | 51.47 | C |
| ATOM | 3451 | C3 | MOO | A | 904 | −36.487 | −5.081 | 26.565 | 1.00 | 48.44 | C |
| ATOM | 3452 | C2 | MOO | A | 904 | −35.000 | −5.214 | 26.875 | 1.00 | 42.95 | C |
| ATOM | 3453 | C1 | MOO | A | 904 | −34.701 | −6.519 | 27.605 | 1.00 | 51.09 | C |
| ATOM | 3454 | C | MOO | A | 904 | −33.220 | −6.663 | 27.878 | 1.00 | 54.93 | C |
| TER | | | | | | | | | | | |

TABLE B

| | | | | | | | | | | | |
|---|---|---|---|---|---|---|---|---|---|---|---|
| ATOM | 3455 | N | HIS | B | 115 | −17.321 | 25.918 | 5.163 | 1.00 | 89.27 | N |
| ATOM | 3456 | CA | HIS | B | 115 | −18.605 | 25.349 | 4.805 | 1.00 | 96.75 | C |
| ATOM | 3457 | CB | HIS | B | 115 | −18.412 | 24.129 | 3.881 | 1.00 | 110.84 | C |
| ATOM | 3458 | CG | HIS | B | 115 | −18.006 | 24.471 | 2.476 | 1.00 | 117.19 | C |
| ATOM | 3459 | ND1 | HIS | B | 115 | −16.732 | 24.885 | 2.145 | 1.00 | 117.67 | N |
| ATOM | 3460 | CE1 | HIS | B | 115 | −16.667 | 25.107 | 0.844 | 1.00 | 114.26 | C |
| ATOM | 3461 | NE2 | HIS | B | 115 | −17.850 | 24.846 | 0.316 | 1.00 | 110.33 | N |
| ATOM | 3462 | CD2 | HIS | B | 115 | −18.703 | 24.442 | 1.315 | 1.00 | 110.29 | C |
| ATOM | 3463 | C | HIS | B | 115 | −19.451 | 24.918 | 6.016 | 1.00 | 83.95 | C |
| ATOM | 3464 | O | HIS | B | 115 | −20.616 | 25.282 | 6.117 | 1.00 | 76.94 | O |
| ATOM | 3465 | N | TYR | B | 116 | −18.837 | 24.204 | 6.968 | 1.00 | 91.91 | N |
| ATOM | 3466 | CA | TYR | B | 116 | −19.559 | 23.822 | 8.192 | 1.00 | 88.36 | C |
| ATOM | 3467 | CB | TYR | B | 116 | −18.794 | 22.757 | 8.989 | 1.00 | 95.32 | C |
| ATOM | 3468 | CG | TYR | B | 116 | −18.633 | 21.466 | 8.209 | 1.00 | 97.75 | C |
| ATOM | 3469 | CD1 | TYR | B | 116 | −19.746 | 20.747 | 7.782 | 1.00 | 88.23 | C |
| ATOM | 3470 | CE1 | TYR | B | 116 | −19.607 | 19.572 | 7.058 | 1.00 | 97.42 | C |
| ATOM | 3471 | CZ | TYR | B | 116 | −18.341 | 19.105 | 6.753 | 1.00 | 100.21 | C |
| ATOM | 3472 | OH | TYR | B | 116 | −18.179 | 17.938 | 6.038 | 1.00 | 83.99 | O |
| ATOM | 3473 | CE2 | TYR | B | 116 | −17.224 | 19.803 | 7.163 | 1.00 | 100.27 | C |
| ATOM | 3474 | CD2 | TYR | B | 116 | −17.373 | 20.977 | 7.884 | 1.00 | 99.99 | C |
| ATOM | 3475 | C | TYR | B | 116 | −20.005 | 25.007 | 9.046 | 1.00 | 78.54 | C |
| ATOM | 3476 | O | TYR | B | 116 | −21.098 | 24.984 | 9.586 | 1.00 | 76.34 | O |
| ATOM | 3477 | N | HIS | B | 117 | −19.122 | 26.005 | 9.240 | 1.00 | 83.32 | N |
| ATOM | 3478 | CA | HIS | B | 117 | −19.487 | 27.174 | 10.062 | 1.00 | 72.44 | C |
| ATOM | 3479 | CB | HIS | B | 117 | −18.310 | 28.143 | 10.174 | 1.00 | 82.88 | C |
| ATOM | 3480 | CG | HIS | B | 117 | −17.048 | 27.507 | 10.663 | 1.00 | 94.58 | C |
| ATOM | 3481 | ND1 | HIS | B | 117 | −15.815 | 28.112 | 10.537 | 1.00 | 101.78 | N |
| ATOM | 3482 | CE1 | HIS | B | 117 | −14.888 | 27.324 | 11.050 | 1.00 | 106.45 | C |
| ATOM | 3483 | NE2 | HIS | B | 117 | −15.474 | 26.231 | 11.506 | 1.00 | 99.31 | N |
| ATOM | 3484 | CD2 | HIS | B | 117 | −16.826 | 26.321 | 11.276 | 1.00 | 89.67 | C |
| ATOM | 3485 | C | HIS | B | 117 | −20.704 | 27.934 | 9.527 | 1.00 | 67.85 | C |
| ATOM | 3486 | O | HIS | B | 117 | −21.618 | 28.265 | 10.250 | 1.00 | 68.40 | O |
| ATOM | 3487 | N | VAL | B | 118 | −20.717 | 28.194 | 8.230 | 1.00 | 71.44 | N |
| ATOM | 3488 | CA | VAL | B | 118 | −21.838 | 28.847 | 7.596 | 1.00 | 66.02 | C |
| ATOM | 3489 | CB | VAL | B | 118 | −21.580 | 29.094 | 6.096 | 1.00 | 70.10 | C |
| ATOM | 3490 | CG1 | VAL | B | 118 | −22.834 | 29.622 | 5.409 | 1.00 | 65.18 | C |
| ATOM | 3491 | CG2 | VAL | B | 118 | −20.431 | 30.070 | 5.925 | 1.00 | 70.59 | C |
| ATOM | 3492 | C | VAL | B | 118 | −23.088 | 28.043 | 7.784 | 1.00 | 64.86 | C |
| ATOM | 3493 | O | VAL | B | 118 | −24.103 | 28.587 | 8.155 | 1.00 | 67.16 | O |
| ATOM | 3494 | N | ALA | B | 119 | −22.975 | 26.726 | 7.584 | 1.00 | 66.57 | N |
| ATOM | 3495 | CA | ALA | B | 119 | −24.091 | 25.797 | 7.778 | 1.00 | 62.70 | C |
| ATOM | 3496 | CB | ALA | B | 119 | −23.655 | 24.375 | 7.455 | 1.00 | 62.94 | C |
| ATOM | 3497 | C | ALA | B | 119 | −24.678 | 25.867 | 9.183 | 1.00 | 56.36 | C |
| ATOM | 3498 | O | ALA | B | 119 | −25.872 | 25.945 | 9.358 | 1.00 | 55.56 | O |
| ATOM | 3499 | N | ALA | B | 120 | −23.832 | 25.939 | 10.182 | 1.00 | 53.72 | N |
| ATOM | 3500 | CA | ALA | B | 120 | −24.310 | 26.096 | 11.537 | 1.00 | 52.65 | C |
| ATOM | 3501 | CB | ALA | B | 120 | −23.138 | 26.079 | 12.509 | 1.00 | 52.31 | C |
| ATOM | 3502 | C | ALA | B | 120 | −25.120 | 27.381 | 11.708 | 1.00 | 51.21 | C |
| ATOM | 3503 | O | ALA | B | 120 | −26.183 | 27.359 | 12.260 | 1.00 | 48.19 | O |
| ATOM | 3504 | N | ILE | B | 121 | −24.626 | 28.499 | 11.192 | 1.00 | 55.52 | N |
| ATOM | 3505 | CA | ILE | B | 121 | −25.303 | 29.790 | 11.321 | 1.00 | 51.43 | C |
| ATOM | 3506 | CB | ILE | B | 121 | −24.484 | 30.916 | 10.662 | 1.00 | 51.42 | C |
| ATOM | 3507 | CG1 | ILE | B | 121 | −23.123 | 31.046 | 11.349 | 1.00 | 58.37 | C |
| ATOM | 3508 | CD1 | ILE | B | 121 | −22.142 | 31.938 | 10.617 | 1.00 | 61.78 | C |
| ATOM | 3509 | CG2 | ILE | B | 121 | −25.236 | 32.236 | 10.718 | 1.00 | 43.33 | C |
| ATOM | 3510 | C | ILE | B | 121 | −26.684 | 29.728 | 10.705 | 1.00 | 54.20 | C |
| ATOM | 3511 | O | ILE | B | 121 | −27.650 | 30.150 | 11.304 | 1.00 | 56.96 | O |
| ATOM | 3512 | N | ILE | B | 122 | −26.782 | 29.106 | 9.545 | 1.00 | 50.28 | N |
| ATOM | 3513 | CA | ILE | B | 122 | −28.062 | 28.890 | 8.903 | 1.00 | 52.76 | C |
| ATOM | 3514 | CB | ILE | B | 122 | −27.882 | 28.198 | 7.544 | 1.00 | 53.95 | C |
| ATOM | 3515 | CG1 | ILE | B | 122 | −27.145 | 29.129 | 6.586 | 1.00 | 49.78 | C |
| ATOM | 3516 | CD1 | ILE | B | 122 | −26.986 | 28.555 | 5.202 | 1.00 | 60.46 | C |
| ATOM | 3517 | CG2 | ILE | B | 122 | −29.228 | 27.786 | 6.955 | 1.00 | 58.39 | C |
| ATOM | 3518 | C | ILE | B | 122 | −29.009 | 28.055 | 9.790 | 1.00 | 53.10 | C |
| ATOM | 3519 | O | ILE | B | 122 | −30.175 | 28.370 | 9.921 | 1.00 | 47.24 | O |
| ATOM | 3520 | N | ASN | B | 123 | −28.483 | 26.998 | 10.407 | 1.00 | 52.50 | N |
| ATOM | 3521 | CA | ASN | B | 123 | −29.272 | 26.109 | 11.242 | 1.00 | 45.26 | C |
| ATOM | 3522 | CB | ASN | B | 123 | −28.421 | 24.925 | 11.714 | 1.00 | 47.85 | C |
| ATOM | 3523 | CG | ASN | B | 123 | −28.606 | 23.684 | 10.856 | 1.00 | 56.59 | C |
| ATOM | 3524 | OD1 | ASN | B | 123 | −29.705 | 23.407 | 10.368 | 1.00 | 48.19 | O |
| ATOM | 3525 | ND2 | ASN | B | 123 | −27.528 | 22.925 | 10.673 | 1.00 | 60.15 | N |
| ATOM | 3526 | C | ASN | B | 123 | −29.841 | 26.827 | 12.452 | 1.00 | 42.24 | C |
| ATOM | 3527 | O | ASN | B | 123 | −30.974 | 26.620 | 12.800 | 1.00 | 37.67 | O |
| ATOM | 3528 | N | TYR | B | 124 | −29.038 | 27.653 | 13.121 | 1.00 | 46.46 | N |
| ATOM | 3529 | CA | TYR | B | 124 | −29.514 | 28.380 | 14.289 | 1.00 | 42.50 | C |
| ATOM | 3530 | CB | TYR | B | 124 | −28.369 | 29.116 | 14.985 | 1.00 | 34.12 | C |
| ATOM | 3531 | CG | TYR | B | 124 | −27.581 | 28.245 | 15.928 | 1.00 | 34.10 | C |
| ATOM | 3532 | CD1 | TYR | B | 124 | −27.964 | 28.103 | 17.254 | 1.00 | 41.86 | C |
| ATOM | 3533 | CE1 | TYR | B | 124 | −27.246 | 27.307 | 18.124 | 1.00 | 41.55 | C |
| ATOM | 3534 | CZ | TYR | B | 124 | −26.130 | 26.641 | 17.671 | 1.00 | 41.35 | C |
| ATOM | 3535 | OH | TYR | B | 124 | −25.412 | 25.845 | 18.534 | 1.00 | 44.27 | O |
| ATOM | 3536 | CE2 | TYR | B | 124 | −25.730 | 26.765 | 16.357 | 1.00 | 43.86 | C |
| ATOM | 3537 | CD2 | TYR | B | 124 | −26.456 | 27.561 | 15.495 | 1.00 | 38.27 | C |
| ATOM | 3538 | C | TYR | B | 124 | −30.600 | 29.355 | 13.937 | 1.00 | 40.99 | C |
| ATOM | 3539 | O | TYR | B | 124 | −31.603 | 29.401 | 14.605 | 1.00 | 43.03 | O |
| ATOM | 3540 | N | LEU | B | 125 | −30.420 | 30.127 | 12.870 | 1.00 | 39.67 | N |
| ATOM | 3541 | CA | LEU | B | 125 | −31.461 | 31.054 | 12.446 | 1.00 | 42.06 | C |
| ATOM | 3542 | CB | LEU | B | 125 | −30.987 | 31.903 | 11.259 | 1.00 | 39.85 | C |
| ATOM | 3543 | CG | LEU | B | 125 | −31.975 | 32.928 | 10.691 | 1.00 | 28.16 | C |
| ATOM | 3544 | CD1 | LEU | B | 125 | −32.418 | 33.922 | 11.752 | 1.00 | 25.86 | C |
| ATOM | 3545 | CD2 | LEU | B | 125 | −31.366 | 33.656 | 9.509 | 1.00 | 26.55 | C |
| ATOM | 3546 | C | LEU | B | 125 | −32.744 | 30.351 | 12.106 | 1.00 | 36.85 | C |
| ATOM | 3547 | O | LEU | B | 125 | −33.798 | 30.748 | 12.514 | 1.00 | 35.29 | O |
| ATOM | 3548 | N | GLY | B | 126 | −32.646 | 29.281 | 11.374 | 1.00 | 37.86 | N |
| ATOM | 3549 | CA | GLY | B | 126 | −33.793 | 28.506 | 11.043 | 1.00 | 38.39 | C |
| ATOM | 3550 | C | GLY | B | 126 | −34.508 | 27.872 | 12.178 | 1.00 | 38.36 | C |
| ATOM | 3551 | O | GLY | B | 126 | −35.711 | 27.946 | 12.284 | 1.00 | 41.03 | O |
| ATOM | 3552 | N | HIS | B | 127 | −33.777 | 27.259 | 13.072 | 1.00 | 38.06 | N |
| ATOM | 3553 | CA | HIS | B | 127 | −34.352 | 26.671 | 14.240 | 1.00 | 35.55 | C |
| ATOM | 3554 | CB | HIS | B | 127 | −33.302 | 25.922 | 15.056 | 1.00 | 39.83 | C |
| ATOM | 3555 | CG | HIS | B | 127 | −33.152 | 24.491 | 14.647 | 1.00 | 41.65 | C |
| ATOM | 3556 | ND1 | HIS | B | 127 | −32.367 | 24.101 | 13.586 | 1.00 | 45.11 | N |
| ATOM | 3557 | CE1 | HIS | B | 127 | −32.444 | 22.788 | 13.447 | 1.00 | 41.74 | C |
| ATOM | 3558 | NE2 | HIS | B | 127 | −33.259 | 22.319 | 14.371 | 1.00 | 36.99 | N |
| ATOM | 3559 | CD2 | HIS | B | 127 | −33.722 | 23.365 | 15.134 | 1.00 | 37.67 | C |
| ATOM | 3560 | C | HIS | B | 127 | −35.042 | 27.699 | 15.079 | 1.00 | 37.81 | C |
| ATOM | 3561 | O | HIS | B | 127 | −36.067 | 27.434 | 15.639 | 1.00 | 39.08 | O |
| ATOM | 3562 | N | CYS | B | 128 | −34.450 | 28.875 | 15.200 | 1.00 | 37.73 | N |
| ATOM | 3563 | CA | CYS | B | 128 | −35.022 | 29.929 | 16.011 | 1.00 | 32.24 | C |
| ATOM | 3564 | CB | CYS | B | 128 | −34.039 | 31.090 | 16.156 | 1.00 | 32.62 | C |
| ATOM | 3565 | SG | CYS | B | 128 | −32.559 | 30.685 | 17.087 | 1.00 | 41.50 | S |
| ATOM | 3566 | C | CYS | B | 128 | −36.330 | 30.440 | 15.429 | 1.00 | 34.91 | C |
| ATOM | 3567 | O | CYS | B | 128 | −37.246 | 30.747 | 16.149 | 1.00 | 39.20 | O |
| ATOM | 3568 | N | ILE | B | 129 | −36.417 | 30.520 | 14.118 | 1.00 | 32.77 | N |
| ATOM | 3569 | CA | ILE | B | 129 | −37.645 | 30.921 | 13.462 | 1.00 | 33.31 | C |
| ATOM | 3570 | CB | ILE | B | 129 | −37.425 | 31.200 | 11.962 | 1.00 | 33.67 | C |
| ATOM | 3571 | CG1 | ILE | B | 129 | −36.434 | 32.351 | 11.782 | 1.00 | 31.42 | C |
| ATOM | 3572 | CD1 | ILE | B | 129 | −35.975 | 32.543 | 10.357 | 1.00 | 30.50 | C |
| ATOM | 3573 | CG2 | ILE | B | 129 | −38.744 | 31.529 | 11.279 | 1.00 | 28.63 | C |
| ATOM | 3574 | C | ILE | B | 129 | −38.741 | 29.898 | 13.653 | 1.00 | 35.55 | C |
| ATOM | 3575 | O | ILE | B | 129 | −39.868 | 30.213 | 13.947 | 1.00 | 38.80 | O |
| ATOM | 3576 | N | SER | B | 130 | −38.386 | 28.646 | 13.481 | 1.00 | 32.98 | N |
| ATOM | 3577 | CA | SER | B | 130 | −39.295 | 27.546 | 13.667 | 1.00 | 32.55 | C |
| ATOM | 3578 | CB | SER | B | 130 | −38.644 | 26.225 | 13.253 | 1.00 | 34.57 | C |
| ATOM | 3579 | OG | SER | B | 130 | −38.431 | 26.185 | 11.858 | 1.00 | 36.95 | O |
| ATOM | 3580 | C | SER | B | 130 | −39.817 | 27.427 | 15.068 | 1.00 | 34.23 | C |
| ATOM | 3581 | O | SER | B | 130 | −40.974 | 27.215 | 15.286 | 1.00 | 33.64 | O |
| ATOM | 3582 | N | LEU | B | 131 | −38.941 | 27.532 | 16.030 | 1.00 | 32.66 | N |
| ATOM | 3583 | CA | LEU | B | 131 | −39.317 | 27.426 | 17.412 | 1.00 | 31.91 | C |
| ATOM | 3584 | CB | LEU | B | 131 | −38.094 | 27.597 | 18.309 | 1.00 | 32.90 | C |
| ATOM | 3585 | CG | LEU | B | 131 | −38.382 | 27.392 | 19.792 | 1.00 | 30.90 | C |
| ATOM | 3586 | CD1 | LEU | B | 131 | −38.876 | 25.972 | 20.024 | 1.00 | 33.63 | C |
| ATOM | 3587 | CD2 | LEU | B | 131 | −37.149 | 27.683 | 20.621 | 1.00 | 28.79 | C |
| ATOM | 3588 | C | LEU | B | 131 | −40.355 | 28.446 | 17.779 | 1.00 | 36.62 | C |
| ATOM | 3589 | O | LEU | B | 131 | −41.368 | 28.123 | 18.344 | 1.00 | 38.97 | O |
| ATOM | 3590 | N | VAL | B | 132 | −40.094 | 29.689 | 17.406 | 1.00 | 35.95 | N |
| ATOM | 3591 | CA | VAL | B | 132 | −41.005 | 30.800 | 17.639 | 1.00 | 33.65 | C |
| ATOM | 3592 | CB | VAL | B | 132 | −40.378 | 32.161 | 17.234 | 1.00 | 30.67 | C |
| ATOM | 3593 | CG1 | VAL | B | 132 | −41.388 | 33.284 | 17.381 | 1.00 | 35.24 | C |
| ATOM | 3594 | CG2 | VAL | B | 132 | −39.160 | 32.456 | 18.083 | 1.00 | 29.67 | C |
| ATOM | 3595 | C | VAL | B | 132 | −42.339 | 30.609 | 16.926 | 1.00 | 33.93 | C |
| ATOM | 3596 | O | VAL | B | 132 | −43.376 | 30.792 | 17.503 | 1.00 | 32.97 | O |
| ATOM | 3597 | N | ALA | B | 133 | −42.299 | 30.190 | 15.674 | 1.00 | 34.59 | N |
| ATOM | 3598 | CA | ALA | B | 133 | −43.490 | 29.900 | 14.893 | 1.00 | 36.26 | C |
| ATOM | 3599 | CB | ALA | B | 133 | −43.093 | 29.509 | 13.470 | 1.00 | 34.96 | C |
| ATOM | 3600 | C | ALA | B | 133 | −44.380 | 28.839 | 15.503 | 1.00 | 34.13 | C |
| ATOM | 3601 | O | ALA | B | 133 | −45.572 | 29.010 | 15.592 | 1.00 | 35.89 | O |
| ATOM | 3602 | N | LEU | B | 134 | −43.767 | 27.775 | 15.988 | 1.00 | 29.04 | N |
| ATOM | 3603 | CA | LEU | B | 134 | −44.443 | 26.738 | 16.721 | 1.00 | 30.43 | C |
| ATOM | 3604 | CB | LEU | B | 134 | −43.474 | 25.605 | 17.066 | 1.00 | 30.67 | C |
| ATOM | 3605 | CG | LEU | B | 134 | −43.138 | 24.568 | 15.997 | 1.00 | 33.04 | C |
| ATOM | 3606 | CD1 | LEU | B | 134 | −41.959 | 23.730 | 16.446 | 1.00 | 35.93 | C |
| ATOM | 3607 | CD2 | LEU | B | 134 | −44.329 | 23.679 | 15.715 | 1.00 | 30.09 | C |
| ATOM | 3608 | C | LEU | B | 134 | −45.095 | 27.247 | 17.992 | 1.00 | 35.89 | C |
| ATOM | 3609 | O | LEU | B | 134 | −46.198 | 26.874 | 18.314 | 1.00 | 36.97 | O |
| ATOM | 3610 | N | LEU | B | 135 | −44.377 | 28.062 | 18.747 | 1.00 | 34.62 | N |
| ATOM | 3611 | CA | LEU | B | 135 | −44.860 | 28.614 | 20.002 | 1.00 | 34.66 | C |
| ATOM | 3612 | CB | LEU | B | 135 | −43.748 | 29.367 | 20.740 | 1.00 | 30.14 | C |
| ATOM | 3613 | CG | LEU | B | 135 | −42.685 | 28.506 | 21.420 | 1.00 | 29.88 | C |
| ATOM | 3614 | CD1 | LEU | B | 135 | −41.612 | 29.387 | 22.007 | 1.00 | 33.27 | C |
| ATOM | 3615 | CD2 | LEU | B | 135 | −43.302 | 27.640 | 22.499 | 1.00 | 28.85 | C |
| ATOM | 3616 | C | LEU | B | 135 | −46.061 | 29.510 | 19.823 | 1.00 | 33.18 | C |
| ATOM | 3617 | O | LEU | B | 135 | −47.008 | 29.444 | 20.572 | 1.00 | 35.49 | O |
| ATOM | 3618 | N | VAL | B | 136 | −46.012 | 30.329 | 18.788 | 1.00 | 28.63 | N |
| ATOM | 3619 | CA | VAL | B | 136 | −47.091 | 31.220 | 18.432 | 1.00 | 32.71 | C |
| ATOM | 3620 | CB | VAL | B | 136 | −46.714 | 32.148 | 17.260 | 1.00 | 31.99 | C |
| ATOM | 3621 | CG1 | VAL | B | 136 | −47.939 | 32.896 | 16.755 | 1.00 | 35.39 | C |
| ATOM | 3622 | CG2 | VAL | B | 136 | −45.643 | 33.127 | 17.689 | 1.00 | 31.28 | C |
| ATOM | 3623 | C | VAL | B | 136 | −48.299 | 30.411 | 18.068 | 1.00 | 38.01 | C |
| ATOM | 3624 | O | VAL | B | 136 | −49.371 | 30.642 | 18.569 | 1.00 | 42.18 | O |
| ATOM | 3625 | N | ALA | B | 137 | −48.081 | 29.381 | 17.276 | 1.00 | 32.01 | N |
| ATOM | 3626 | CA | ALA | B | 137 | −49.124 | 28.487 | 16.906 | 1.00 | 33.90 | C |
| ATOM | 3627 | CB | ALA | B | 137 | −48.610 | 27.480 | 15.881 | 1.00 | 31.81 | C |
| ATOM | 3628 | C | ALA | B | 137 | −49.706 | 27.765 | 18.100 | 1.00 | 40.67 | C |
| ATOM | 3629 | O | ALA | B | 137 | −50.894 | 27.667 | 18.233 | 1.00 | 46.27 | O |
| ATOM | 3630 | N | PHE | B | 138 | −48.877 | 27.305 | 19.004 | 1.00 | 38.92 | N |
| ATOM | 3631 | CA | PHE | B | 138 | −49.343 | 26.664 | 20.210 | 1.00 | 39.33 | C |
| ATOM | 3632 | CB | PHE | B | 138 | −48.119 | 26.211 | 21.005 | 1.00 | 33.73 | C |
| ATOM | 3633 | CG | PHE | B | 138 | −48.435 | 25.308 | 22.156 | 1.00 | 38.45 | C |
| ATOM | 3634 | CD1 | PHE | B | 138 | −48.838 | 24.001 | 21.939 | 1.00 | 34.31 | C |
| ATOM | 3635 | CE1 | PHE | B | 138 | −49.118 | 23.167 | 23.002 | 1.00 | 38.56 | C |
| ATOM | 3636 | CZ | PHE | B | 138 | −48.989 | 23.632 | 24.293 | 1.00 | 35.81 | C |
| ATOM | 3637 | CE2 | PHE | B | 138 | −48.578 | 24.930 | 24.519 | 1.00 | 32.40 | C |
| ATOM | 3638 | CD2 | PHE | B | 138 | −48.301 | 25.758 | 23.458 | 1.00 | 33.70 | C |
| ATOM | 3639 | C | PHE | B | 138 | −50.222 | 27.593 | 21.057 | 1.00 | 42.64 | C |
| ATOM | 3640 | O | PHE | B | 138 | −51.289 | 27.237 | 21.499 | 1.00 | 41.95 | O |
| ATOM | 3641 | N | VAL | B | 139 | −49.798 | 28.818 | 21.242 | 1.00 | 39.52 | N |
| ATOM | 3642 | CA | VAL | B | 139 | −50.612 | 29.809 | 21.919 | 1.00 | 42.72 | C |
| ATOM | 3643 | CB | VAL | B | 139 | −49.916 | 31.177 | 21.998 | 1.00 | 47.41 | C |
| ATOM | 3644 | CG1 | VAL | B | 139 | −50.856 | 32.214 | 22.592 | 1.00 | 49.60 | C |
| ATOM | 3645 | CG2 | VAL | B | 139 | −48.661 | 31.067 | 22.836 | 1.00 | 47.27 | C |
| ATOM | 3646 | C | VAL | B | 139 | −51.990 | 29.965 | 21.280 | 1.00 | 47.43 | C |
| ATOM | 3647 | O | VAL | B | 139 | −52.997 | 29.911 | 21.958 | 1.00 | 53.31 | O |
| ATOM | 3648 | N | LEU | B | 140 | −52.032 | 30.100 | 19.961 | 1.00 | 45.63 | N |
| ATOM | 3649 | CA | LEU | B | 140 | −53.294 | 30.204 | 19.233 | 1.00 | 46.60 | C |
| ATOM | 3650 | CB | LEU | B | 140 | −53.042 | 30.290 | 17.726 | 1.00 | 51.61 | C |
| ATOM | 3651 | CG | LEU | B | 140 | −52.238 | 31.481 | 17.202 | 1.00 | 47.79 | C |
| ATOM | 3652 | CD1 | LEU | B | 140 | −52.280 | 31.518 | 15.680 | 1.00 | 48.85 | C |
| ATOM | 3653 | CD2 | LEU | B | 140 | −52.742 | 32.788 | 17.799 | 1.00 | 56.06 | C |
| ATOM | 3654 | C | LEU | B | 140 | −54.250 | 29.045 | 19.526 | 1.00 | 51.65 | C |
| ATOM | 3655 | O | LEU | B | 140 | −55.428 | 29.264 | 19.806 | 1.00 | 62.32 | O |
| ATOM | 3656 | N | PHE | B | 141 | −53.732 | 27.815 | 19.499 | 1.00 | 46.92 | N |
| ATOM | 3657 | CA | PHE | B | 141 | −54.564 | 26.642 | 19.771 | 1.00 | 51.50 | C |
| ATOM | 3658 | CB | PHE | B | 141 | −53.820 | 25.349 | 19.450 | 1.00 | 44.81 | C |
| ATOM | 3659 | CG | PHE | B | 141 | −53.897 | 24.967 | 18.008 | 1.00 | 49.82 | C |
| ATOM | 3660 | CD2 | PHE | B | 141 | −54.868 | 24.088 | 17.562 | 1.00 | 49.43 | C |
| ATOM | 3661 | CE2 | PHE | B | 141 | −54.947 | 23.745 | 16.230 | 1.00 | 53.68 | C |
| ATOM | 3662 | CZ | PHE | B | 141 | −54.057 | 24.283 | 15.325 | 1.00 | 63.06 | C |
| ATOM | 3663 | CE1 | PHE | B | 141 | −53.092 | 25.167 | 15.755 | 1.00 | 59.17 | C |
| ATOM | 3664 | CD1 | PHE | B | 141 | −53.017 | 25.508 | 17.090 | 1.00 | 49.76 | C |
| ATOM | 3665 | C | PHE | B | 141 | −55.115 | 26.623 | 21.193 | 1.00 | 50.38 | C |
| ATOM | 3666 | O | PHE | B | 141 | −56.214 | 26.185 | 21.423 | 1.00 | 57.35 | O |
| ATOM | 3667 | N | LEU | B | 142 | −54.336 | 27.064 | 22.152 | 1.00 | 47.89 | N |
| ATOM | 3668 | CA | LEU | B | 142 | −54.796 | 27.134 | 23.523 | 1.00 | 50.92 | C |
| ATOM | 3669 | CB | LEU | B | 142 | −53.618 | 27.370 | 24.471 | 1.00 | 49.40 | C |
| ATOM | 3670 | CG | LEU | B | 142 | −52.528 | 26.298 | 24.418 | 1.00 | 41.82 | C |
| ATOM | 3671 | CD1 | LEU | B | 142 | −51.550 | 26.481 | 25.562 | 1.00 | 42.67 | C |
| ATOM | 3672 | CD2 | LEU | B | 142 | −53.136 | 24.907 | 24.444 | 1.00 | 39.97 | C |
| ATOM | 3673 | C | LEU | B | 142 | −55.913 | 28.156 | 23.787 | 1.00 | 60.51 | C |
| ATOM | 3674 | O | LEU | B | 142 | −56.863 | 27.883 | 24.503 | 1.00 | 63.39 | O |
| ATOM | 3675 | N | ARG | B | 143 | −55.774 | 29.358 | 23.243 | 1.00 | 60.87 | N |
| ATOM | 3676 | CA | ARG | B | 143 | −56.776 | 30.409 | 23.450 | 1.00 | 68.12 | C |
| ATOM | 3677 | CB | ARG | B | 143 | −56.277 | 31.822 | 23.098 | 1.00 | 73.47 | C |
| ATOM | 3678 | CG | ARG | B | 143 | −57.296 | 32.889 | 23.543 | 1.00 | 106.64 | C |
| ATOM | 3679 | CD | ARG | B | 143 | −56.902 | 34.328 | 23.281 | 1.00 | 112.85 | C |
| ATOM | 3680 | NE | ARG | B | 143 | −58.041 | 35.241 | 23.421 | 1.00 | 126.72 | N |
| ATOM | 3681 | CZ | ARG | B | 143 | −58.923 | 35.507 | 22.457 | 1.00 | 132.68 | C |
| ATOM | 3682 | NH1 | ARG | B | 143 | −58.818 | 34.929 | 21.264 | 1.00 | 125.30 | N |
| ATOM | 3683 | NH2 | ARG | B | 143 | −59.919 | 36.355 | 22.686 | 1.00 | 136.79 | N |
| ATOM | 3684 | C | ARG | B | 143 | −58.153 | 30.123 | 22.799 | 1.00 | 70.90 | C |
| ATOM | 3685 | O | ARG | B | 143 | −59.188 | 30.511 | 23.330 | 1.00 | 82.77 | O |
| ATOM | 3686 | N | ALA | B | 144 | −58.144 | 29.504 | 21.621 | 1.00 | 65.17 | N |
| ATOM | 3687 | CA | ALA | B | 144 | −59.357 | 29.240 | 20.831 | 1.00 | 72.59 | C |
| ATOM | 3688 | CB | ALA | B | 144 | −58.973 | 28.791 | 19.428 | 1.00 | 78.15 | C |
| ATOM | 3689 | C | ALA | B | 144 | −60.383 | 28.257 | 21.449 | 1.00 | 80.11 | C |
| ATOM | 3690 | O | ALA | B | 144 | −60.016 | 27.324 | 22.151 | 1.00 | 73.74 | O |
| ATOM | 3691 | O | ARG | B | 145 | −62.987 | 24.992 | 20.978 | 1.00 | 88.71 | O |
| ATOM | 3692 | N | ARG | B | 145 | −61.689 | 28.450 | 21.067 | 1.00 | 82.78 | N |
| ATOM | 3693 | CA | ARG | B | 145 | −62.798 | 27.457 | 21.296 | 1.00 | 83.43 | C |
| ATOM | 3694 | C | ARG | B | 145 | −62.542 | 26.097 | 20.591 | 1.00 | 82.83 | C |
| ATOM | 3695 | CB | ARG | B | 145 | −64.099 | 27.983 | 20.690 | 1.00 | 86.80 | C |
| ATOM | 3696 | CG | ARG | B | 145 | −64.754 | 29.192 | 21.325 | 1.00 | 104.35 | C |
| ATOM | 3697 | CD | ARG | B | 145 | −65.925 | 29.628 | 20.429 | 1.00 | 101.87 | C |
| ATOM | 3698 | NE | ARG | B | 145 | −66.906 | 30.468 | 21.114 | 1.00 | 110.71 | N |
| ATOM | 3699 | CZ | ARG | B | 145 | −68.217 | 30.234 | 21.120 | 1.00 | 111.83 | C |
| ATOM | 3700 | NH1 | ARG | B | 145 | −68.710 | 29.185 | 20.474 | 1.00 | 95.83 | N |
| ATOM | 3701 | NH2 | ARG | B | 145 | −69.040 | 31.047 | 21.770 | 1.00 | 114.62 | N |
| ATOM | 3702 | O | SER | B | 146 | −60.798 | 22.783 | 19.159 | 1.00 | 83.42 | O |
| ATOM | 3703 | N | SER | B | 146 | −61.788 | 26.204 | 19.521 | 1.00 | 75.82 | N |
| ATOM | 3704 | CA | SER | B | 146 | −61.440 | 25.072 | 18.725 | 1.00 | 77.84 | C |
| ATOM | 3705 | C | SER | B | 146 | −60.765 | 23.935 | 19.557 | 1.00 | 82.35 | C |
| ATOM | 3706 | CB | SER | B | 146 | −60.555 | 25.498 | 17.548 | 1.00 | 79.40 | C |
| ATOM | 3707 | OG | SER | B | 146 | −59.299 | 25.981 | 17.994 | 1.00 | 84.68 | O |
| ATOM | 3708 | N | ILE | B | 147 | −60.107 | 24.308 | 20.678 | 1.00 | 80.82 | N |
| ATOM | 3709 | CA | ILE | B | 147 | −59.390 | 23.392 | 21.563 | 1.00 | 74.96 | C |
| ATOM | 3710 | CB | ILE | B | 147 | −58.585 | 24.119 | 22.669 | 1.00 | 72.05 | C |
| ATOM | 3711 | CG1 | ILE | B | 147 | −57.528 | 23.174 | 23.247 | 1.00 | 69.69 | C |
| ATOM | 3712 | CD1 | ILE | B | 147 | −56.704 | 23.770 | 24.374 | 1.00 | 69.10 | C |
| ATOM | 3713 | CG2 | ILE | B | 147 | −59.500 | 24.662 | 23.758 | 1.00 | 69.55 | C |
| ATOM | 3714 | C | ILE | B | 147 | −60.277 | 22.307 | 22.145 | 1.00 | 74.19 | C |
| ATOM | 3715 | O | ILE | B | 147 | −59.849 | 21.201 | 22.408 | 1.00 | 74.80 | O |
| ATOM | 3716 | N | ARG | B | 148 | −61.515 | 22.650 | 22.397 | 1.00 | 75.33 | N |
| ATOM | 3717 | CA | ARG | B | 148 | −62.510 | 21.696 | 22.876 | 1.00 | 78.84 | C |
| ATOM | 3718 | C | ARG | B | 148 | −62.650 | 20.479 | 21.920 | 1.00 | 67.19 | C |
| ATOM | 3719 | O | ARG | B | 148 | −62.790 | 19.343 | 22.366 | 1.00 | 55.14 | O |
| ATOM | 3720 | CB | ARG | B | 148 | −63.840 | 22.408 | 23.111 | 1.00 | 88.48 | C |
| ATOM | 3721 | CG | ARG | B | 148 | −63.718 | 23.553 | 24.108 | 1.00 | 97.06 | C |
| ATOM | 3722 | CD | ARG | B | 148 | −65.000 | 24.356 | 24.227 | 1.00 | 109.49 | C |
| ATOM | 3723 | NE | ARG | B | 148 | −64.824 | 25.502 | 25.116 | 1.00 | 121.07 | N |
| ATOM | 3724 | CZ | ARG | B | 148 | −65.817 | 26.263 | 25.565 | 1.00 | 121.49 | C |
| ATOM | 3725 | NH1 | ARG | B | 148 | −67.069 | 25.998 | 25.215 | 1.00 | 119.74 | N |
| ATOM | 3726 | NH2 | ARG | B | 148 | −65.556 | 27.290 | 26.365 | 1.00 | 119.17 | N |
| ATOM | 3727 | N | CYS | B | 149 | −62.675 | 20.752 | 20.597 | 1.00 | 63.02 | N |
| ATOM | 3728 | CA | CYS | B | 149 | −62.710 | 19.634 | 19.614 | 1.00 | 55.27 | C |
| ATOM | 3729 | CB | CYS | B | 149 | −62.835 | 20.195 | 18.196 | 1.00 | 46.09 | C |
| ATOM | 3730 | SG | CYS | B | 149 | −63.983 | 21.574 | 18.031 | 1.00 | 70.16 | S |
| ATOM | 3731 | C | CYS | B | 149 | −61.486 | 18.729 | 19.677 | 1.00 | 53.75 | C |
| ATOM | 3732 | O | CYS | B | 149 | −60.394 | 19.220 | 19.756 | 1.00 | 60.13 | O |
| ATOM | 3733 | N | LEU | B | 150 | −61.678 | 17.408 | 19.503 | 1.00 | 49.76 | N |
| ATOM | 3734 | CA | LEU | B | 150 | −60.591 | 16.425 | 19.315 | 1.00 | 43.64 | C |
| ATOM | 3735 | CB | LEU | B | 150 | −61.178 | 15.034 | 19.053 | 1.00 | 36.89 | C |
| ATOM | 3736 | CG | LEU | B | 150 | −60.183 | 13.878 | 18.977 | 1.00 | 36.20 | C |
| ATOM | 3737 | CD1 | LEU | B | 150 | −59.423 | 13.768 | 20.281 | 1.00 | 40.06 | C |
| ATOM | 3738 | CD2 | LEU | B | 150 | −60.888 | 12.574 | 18.664 | 1.00 | 36.20 | C |
| ATOM | 3739 | C | LEU | B | 150 | −59.592 | 16.771 | 18.227 | 1.00 | 47.13 | C |
| ATOM | 3740 | O | LEU | B | 150 | −58.407 | 16.569 | 18.359 | 1.00 | 45.80 | O |
| ATOM | 3741 | N | ARG | B | 151 | −60.088 | 17.309 | 17.136 | 1.00 | 50.90 | N |
| ATOM | 3742 | CA | ARG | B | 151 | −59.264 | 17.676 | 16.000 | 1.00 | 48.79 | C |
| ATOM | 3743 | CB | ARG | B | 151 | −60.117 | 18.363 | 14.930 | 1.00 | 53.33 | C |
| ATOM | 3744 | CG | ARG | B | 151 | −59.358 | 18.746 | 13.675 | 1.00 | 59.69 | C |
| ATOM | 3745 | CD | ARG | B | 151 | −60.295 | 19.279 | 12.609 | 1.00 | 65.78 | C |
| ATOM | 3746 | NE | ARG | B | 151 | −61.063 | 20.426 | 13.080 | 1.00 | 72.68 | N |
| ATOM | 3747 | CZ | ARG | B | 151 | −60.604 | 21.672 | 13.093 | 1.00 | 84.00 | C |
| ATOM | 3748 | NH1 | ARG | B | 151 | −59.371 | 21.932 | 12.670 | 1.00 | 83.51 | N |
| ATOM | 3749 | NH2 | ARG | B | 151 | −61.373 | 22.660 | 13.533 | 1.00 | 81.75 | N |
| ATOM | 3750 | C | ARG | B | 151 | −58.116 | 18.580 | 16.425 | 1.00 | 51.60 | C |
| ATOM | 3751 | O | ARG | B | 151 | −56.960 | 18.303 | 16.164 | 1.00 | 52.88 | O |
| ATOM | 3752 | N | ASN | B | 152 | −58.477 | 19.613 | 17.171 | 1.00 | 45.82 | N |
| ATOM | 3753 | CA | ASN | B | 152 | −57.536 | 20.552 | 17.737 | 1.00 | 50.81 | C |
| ATOM | 3754 | CB | ASN | B | 152 | −58.210 | 21.868 | 18.080 | 1.00 | 55.66 | C |
| ATOM | 3755 | CG | ASN | B | 152 | −58.802 | 22.533 | 16.858 | 1.00 | 61.43 | C |
| ATOM | 3756 | OD1 | ASN | B | 152 | −59.919 | 22.218 | 16.447 | 1.00 | 70.40 | O |
| ATOM | 3757 | ND2 | ASN | B | 152 | −58.044 | 23.435 | 16.250 | 1.00 | 62.75 | N |
| ATOM | 3758 | C | ASN | B | 152 | −56.708 | 19.995 | 18.888 | 1.00 | 51.21 | C |
| ATOM | 3759 | O | ASN | B | 152 | −55.573 | 20.374 | 19.057 | 1.00 | 49.02 | O |
| ATOM | 3760 | N | ILE | B | 153 | −57.264 | 19.103 | 19.706 | 1.00 | 48.18 | N |
| ATOM | 3761 | CA | ILE | B | 153 | −56.470 | 18.464 | 20.755 | 1.00 | 42.03 | C |
| ATOM | 3762 | CB | ILE | B | 153 | −57.293 | 17.476 | 21.580 | 1.00 | 38.38 | C |
| ATOM | 3763 | CG1 | ILE | B | 153 | −58.262 | 18.237 | 22.482 | 1.00 | 41.85 | C |
| ATOM | 3764 | CD1 | ILE | B | 153 | −59.053 | 17.346 | 23.412 | 1.00 | 42.09 | C |
| ATOM | 3765 | CG2 | ILE | B | 153 | −56.383 | 16.595 | 22.416 | 1.00 | 36.83 | C |
| ATOM | 3766 | C | ILE | B | 153 | −55.260 | 17.755 | 20.148 | 1.00 | 46.96 | C |
| ATOM | 3767 | O | ILE | B | 153 | −54.151 | 17.927 | 20.603 | 1.00 | 49.87 | O |
| ATOM | 3768 | N | ILE | B | 154 | −55.484 | 17.033 | 19.045 | 1.00 | 46.50 | N |
| ATOM | 3769 | CA | ILE | B | 154 | −54.414 | 16.396 | 18.300 | 1.00 | 40.50 | C |
| ATOM | 3770 | CB | ILE | B | 154 | −54.943 | 15.458 | 17.216 | 1.00 | 41.03 | C |
| ATOM | 3771 | CG1 | ILE | B | 154 | −55.810 | 14.385 | 17.863 | 1.00 | 44.36 | C |
| ATOM | 3772 | CD1 | ILE | B | 154 | −56.318 | 13.367 | 16.888 | 1.00 | 50.72 | C |
| ATOM | 3773 | CG2 | ILE | B | 154 | −53.799 | 14.789 | 16.481 | 1.00 | 40.41 | C |
| ATOM | 3774 | C | ILE | B | 154 | −53.439 | 17.399 | 17.742 | 1.00 | 40.57 | C |
| ATOM | 3775 | O | ILE | B | 154 | −52.263 | 17.219 | 17.877 | 1.00 | 43.04 | O |
| ATOM | 3776 | N | HIS | B | 155 | −53.914 | 18.463 | 17.120 | 1.00 | 41.63 | N |
| ATOM | 3777 | CA | HIS | B | 155 | −53.005 | 19.470 | 16.574 | 1.00 | 40.62 | C |
| ATOM | 3778 | CB | HIS | B | 155 | −53.772 | 20.562 | 15.832 | 1.00 | 46.69 | C |
| ATOM | 3779 | CG | HIS | B | 155 | −54.401 | 20.095 | 14.555 | 1.00 | 50.06 | C |
| ATOM | 3780 | ND1 | HIS | B | 155 | −53.672 | 19.532 | 13.527 | 1.00 | 52.19 | N |
| ATOM | 3781 | CE1 | HIS | B | 155 | −54.488 | 19.218 | 12.538 | 1.00 | 50.65 | C |
| ATOM | 3782 | NE2 | HIS | B | 155 | −55.717 | 19.556 | 12.883 | 1.00 | 44.41 | N |
| ATOM | 3783 | CD2 | HIS | B | 155 | −55.690 | 20.101 | 14.144 | 1.00 | 45.29 | C |
| ATOM | 3784 | C | HIS | B | 155 | −52.070 | 20.076 | 17.628 | 1.00 | 39.08 | C |
| ATOM | 3785 | O | HIS | B | 155 | −50.885 | 20.108 | 17.455 | 1.00 | 39.50 | O |
| ATOM | 3786 | N | ALA | B | 156 | −52.606 | 20.494 | 18.764 | 1.00 | 42.58 | N |
| ATOM | 3787 | CA | ALA | B | 156 | −51.807 | 21.129 | 19.811 | 1.00 | 38.94 | C |
| ATOM | 3788 | CB | ALA | B | 156 | −52.709 | 21.664 | 20.926 | 1.00 | 27.64 | C |
| ATOM | 3789 | C | ALA | B | 156 | −50.730 | 20.212 | 20.387 | 1.00 | 40.93 | C |
| ATOM | 3790 | O | ALA | B | 156 | −49.617 | 20.612 | 20.631 | 1.00 | 36.67 | O |
| ATOM | 3791 | N | ASN | B | 157 | −51.075 | 18.952 | 20.574 | 1.00 | 40.88 | N |
| ATOM | 3792 | CA | ASN | B | 157 | −50.120 | 17.917 | 20.925 | 1.00 | 38.72 | C |
| ATOM | 3793 | CB | ASN | B | 157 | −50.831 | 16.575 | 21.086 | 1.00 | 36.55 | C |
| ATOM | 3794 | CG | ASN | B | 157 | −51.445 | 16.405 | 22.452 | 1.00 | 38.23 | C |
| ATOM | 3795 | OD1 | ASN | B | 157 | −50.788 | 15.957 | 23.389 | 1.00 | 45.05 | O |
| ATOM | 3796 | ND2 | ASN | B | 157 | −52.713 | 16.761 | 22.576 | 1.00 | 44.31 | N |
| ATOM | 3797 | C | ASN | B | 157 | −49.009 | 17.773 | 19.910 | 1.00 | 35.70 | C |
| ATOM | 3798 | O | ASN | B | 157 | −47.874 | 17.657 | 20.273 | 1.00 | 33.74 | O |
| ATOM | 3799 | N | LEU | B | 158 | −49.358 | 17.794 | 18.632 | 1.00 | 35.01 | N |
| ATOM | 3800 | CA | LEU | B | 158 | −48.412 | 17.720 | 17.530 | 1.00 | 35.54 | C |
| ATOM | 3801 | CB | LEU | B | 158 | −49.162 | 17.562 | 16.207 | 1.00 | 34.21 | C |
| ATOM | 3802 | CG | LEU | B | 158 | −48.342 | 17.642 | 14.923 | 1.00 | 36.46 | C |
| ATOM | 3803 | CD1 | LEU | B | 158 | −47.341 | 16.516 | 14.876 | 1.00 | 42.73 | C |
| ATOM | 3804 | CD2 | LEU | B | 158 | −49.254 | 17.585 | 13.714 | 1.00 | 48.62 | C |
| ATOM | 3805 | C | LEU | B | 158 | −47.478 | 18.925 | 17.475 | 1.00 | 35.15 | C |
| ATOM | 3806 | O | LEU | B | 158 | −46.287 | 18.800 | 17.368 | 1.00 | 34.08 | O |
| ATOM | 3807 | N | ILE | B | 159 | −48.017 | 20.112 | 17.640 | 1.00 | 36.29 | N |
| ATOM | 3808 | CA | ILE | B | 159 | −47.210 | 21.312 | 17.693 | 1.00 | 35.09 | C |
| ATOM | 3809 | CB | ILE | B | 159 | −48.085 | 22.579 | 17.761 | 1.00 | 35.09 | C |
| ATOM | 3810 | CG1 | ILE | B | 159 | −49.092 | 22.592 | 16.614 | 1.00 | 38.17 | C |
| ATOM | 3811 | CD1 | ILE | B | 159 | −50.035 | 23.769 | 16.662 | 1.00 | 41.17 | C |
| ATOM | 3812 | CG2 | ILE | B | 159 | −47.231 | 23.833 | 17.712 | 1.00 | 31.45 | C |
| ATOM | 3813 | C | ILE | B | 159 | −46.271 | 21.273 | 18.894 | 1.00 | 38.28 | C |
| ATOM | 3814 | O | ILE | B | 159 | −45.111 | 21.553 | 18.761 | 1.00 | 41.02 | O |
| ATOM | 3815 | N | ALA | B | 160 | −46.773 | 20.844 | 20.046 | 1.00 | 37.37 | N |
| ATOM | 3816 | CA | ALA | B | 160 | −45.980 | 20.626 | 21.261 | 1.00 | 34.92 | C |
| ATOM | 3817 | CB | ALA | B | 160 | −46.875 | 20.159 | 22.409 | 1.00 | 36.60 | C |
| ATOM | 3818 | C | ALA | B | 160 | −44.839 | 19.652 | 21.046 | 1.00 | 30.63 | C |
| ATOM | 3819 | O | ALA | B | 160 | −43.737 | 19.876 | 21.453 | 1.00 | 27.21 | O |
| ATOM | 3820 | N | ALA | B | 161 | −45.117 | 18.575 | 20.372 | 1.00 | 31.69 | N |
| ATOM | 3821 | CA | ALA | B | 161 | −44.142 | 17.569 | 20.059 | 1.00 | 29.94 | C |
| ATOM | 3822 | CB | ALA | B | 161 | −44.800 | 16.422 | 19.313 | 1.00 | 32.90 | C |
| ATOM | 3823 | C | ALA | B | 161 | −42.987 | 18.138 | 19.242 | 1.00 | 30.00 | C |
| ATOM | 3824 | O | ALA | B | 161 | −41.837 | 17.926 | 19.538 | 1.00 | 32.82 | O |
| ATOM | 3825 | N | PHE | B | 162 | −43.328 | 18.934 | 18.249 | 1.00 | 29.42 | N |
| ATOM | 3826 | CA | PHE | B | 162 | −42.367 | 19.665 | 17.438 | 1.00 | 29.25 | C |
| ATOM | 3827 | CB | PHE | B | 162 | −43.004 | 20.206 | 16.161 | 1.00 | 28.95 | C |
| ATOM | 3828 | CG | PHE | B | 162 | −43.052 | 19.191 | 15.054 | 1.00 | 26.35 | C |
| ATOM | 3829 | CD2 | PHE | B | 162 | −42.072 | 19.169 | 14.083 | 1.00 | 27.39 | C |
| ATOM | 3830 | CE2 | PHE | B | 162 | −42.099 | 18.231 | 13.075 | 1.00 | 25.20 | C |
| ATOM | 3831 | CZ | PHE | B | 162 | −43.107 | 17.295 | 13.026 | 1.00 | 24.18 | C |
| ATOM | 3832 | CE1 | PHE | B | 162 | −44.084 | 17.297 | 13.987 | 1.00 | 27.82 | C |
| ATOM | 3833 | CD1 | PHE | B | 162 | −44.054 | 18.238 | 15.002 | 1.00 | 27.83 | C |
| ATOM | 3834 | C | PHE | B | 162 | −41.590 | 20.727 | 18.240 | 1.00 | 34.34 | C |
| ATOM | 3835 | O | PHE | B | 162 | −40.408 | 20.888 | 18.066 | 1.00 | 35.39 | O |
| ATOM | 3836 | N | ILE | B | 163 | −42.264 | 21.432 | 19.145 | 1.00 | 31.97 | N |
| ATOM | 3837 | CA | ILE | B | 163 | −41.638 | 22.435 | 20.014 | 1.00 | 32.26 | C |
| ATOM | 3838 | CB | ILE | B | 163 | −42.675 | 23.095 | 20.944 | 1.00 | 29.85 | C |
| ATOM | 3839 | CG1 | ILE | B | 163 | −43.643 | 23.953 | 20.133 | 1.00 | 28.75 | C |
| ATOM | 3840 | CD1 | ILE | B | 163 | −44.783 | 24.523 | 20.946 | 1.00 | 26.59 | C |
| ATOM | 3841 | CG2 | ILE | B | 163 | −41.990 | 23.944 | 22.006 | 1.00 | 26.88 | C |
| ATOM | 3842 | C | ILE | B | 163 | −40.525 | 21.846 | 20.861 | 1.00 | 32.44 | C |
| ATOM | 3843 | O | ILE | B | 163 | −39.465 | 22.405 | 20.984 | 1.00 | 28.82 | O |
| ATOM | 3844 | N | LEU | B | 164 | −40.785 | 20.698 | 21.443 | 1.00 | 30.76 | N |
| ATOM | 3845 | CA | LEU | B | 164 | −39.824 | 20.016 | 22.243 | 1.00 | 23.53 | C |
| ATOM | 3846 | CB | LEU | B | 164 | −40.482 | 18.886 | 23.035 | 1.00 | 27.09 | C |
| ATOM | 3847 | CG | LEU | B | 164 | −41.564 | 19.314 | 24.032 | 1.00 | 29.22 | C |
| ATOM | 3848 | CD1 | LEU | B | 164 | −42.231 | 18.098 | 24.647 | 1.00 | 27.63 | C |
| ATOM | 3849 | CD2 | LEU | B | 164 | −40.989 | 20.215 | 25.115 | 1.00 | 26.02 | C |
| ATOM | 3850 | C | LEU | B | 164 | −38.657 | 19.483 | 21.437 | 1.00 | 30.01 | C |
| ATOM | 3851 | O | LEU | B | 164 | −37.538 | 19.646 | 21.839 | 1.00 | 37.39 | O |
| ATOM | 3852 | N | ARG | B | 165 | −38.878 | 18.901 | 20.267 | 1.00 | 29.11 | N |
| ATOM | 3853 | CA | ARG | B | 165 | −37.757 | 18.597 | 19.382 | 1.00 | 28.07 | C |
| ATOM | 3854 | CB | ARG | B | 165 | −38.193 | 17.868 | 18.110 | 1.00 | 31.20 | C |
| ATOM | 3855 | CG | ARG | B | 165 | −37.016 | 17.602 | 17.180 | 1.00 | 33.84 | C |
| ATOM | 3856 | CD | ARG | B | 165 | −37.351 | 16.620 | 16.080 | 1.00 | 41.67 | C |
| ATOM | 3857 | NE | ARG | B | 165 | −36.221 | 16.406 | 15.177 | 1.00 | 44.88 | N |
| ATOM | 3858 | CZ | ARG | B | 165 | −35.225 | 15.559 | 15.413 | 1.00 | 40.61 | C |
| ATOM | 3859 | NH1 | ARG | B | 165 | −35.209 | 14.850 | 16.533 | 1.00 | 41.38 | N |
| ATOM | 3860 | NH2 | ARG | B | 165 | −34.242 | 15.421 | 14.535 | 1.00 | 44.70 | N |
| ATOM | 3861 | C | ARG | B | 165 | −36.837 | 19.791 | 19.024 | 1.00 | 34.45 | C |
| ATOM | 3862 | O | ARG | B | 165 | −35.637 | 19.696 | 19.143 | 1.00 | 38.40 | O |
| ATOM | 3863 | N | ASN | B | 166 | −37.399 | 20.932 | 18.608 | 1.00 | 36.24 | N |
| ATOM | 3864 | CA | ASN | B | 166 | −36.614 | 22.136 | 18.269 | 1.00 | 35.53 | C |
| ATOM | 3865 | CB | ASN | B | 166 | −37.504 | 23.213 | 17.639 | 1.00 | 39.19 | C |
| ATOM | 3866 | CG | ASN | B | 166 | −37.959 | 22.834 | 16.237 | 1.00 | 50.15 | C |
| ATOM | 3867 | OD1 | ASN | B | 166 | −38.717 | 21.875 | 16.058 | 1.00 | 45.95 | O |
| ATOM | 3868 | ND2 | ASN | B | 166 | −37.502 | 23.583 | 15.237 | 1.00 | 43.30 | N |
| ATOM | 3869 | C | ASN | B | 166 | −35.837 | 22.680 | 19.448 | 1.00 | 35.55 | C |
| ATOM | 3870 | O | ASN | B | 166 | −34.692 | 23.024 | 19.339 | 1.00 | 41.85 | O |
| ATOM | 3871 | N | ALA | B | 167 | −36.473 | 22.755 | 20.590 | 1.00 | 34.69 | N |
| ATOM | 3872 | CA | ALA | B | 167 | −35.815 | 23.160 | 21.808 | 1.00 | 31.86 | C |
| ATOM | 3873 | CB | ALA | B | 167 | −36.831 | 23.295 | 22.936 | 1.00 | 33.11 | C |
| ATOM | 3874 | C | ALA | B | 167 | −34.681 | 22.206 | 22.228 | 1.00 | 33.55 | C |
| ATOM | 3875 | O | ALA | B | 167 | −33.586 | 22.611 | 22.569 | 1.00 | 37.32 | O |
| ATOM | 3876 | N | THR | B | 168 | −34.957 | 20.908 | 22.136 | 1.00 | 31.40 | N |
| ATOM | 3877 | CA | THR | B | 168 | −33.985 | 19.853 | 22.362 | 1.00 | 28.05 | C |
| ATOM | 3878 | CB | THR | B | 168 | −34.587 | 18.442 | 22.281 | 1.00 | 29.24 | C |
| ATOM | 3879 | OG1 | THR | B | 168 | −35.711 | 18.364 | 23.162 | 1.00 | 31.54 | O |
| ATOM | 3880 | CG2 | THR | B | 168 | −33.564 | 17.410 | 22.712 | 1.00 | 26.75 | C |
| ATOM | 3881 | C | THR | B | 168 | −32.761 | 19.992 | 21.485 | 1.00 | 35.88 | C |
| ATOM | 3882 | O | THR | B | 168 | −31.667 | 19.775 | 21.939 | 1.00 | 40.02 | O |
| ATOM | 3883 | N | TRP | B | 169 | −32.950 | 20.373 | 20.218 | 1.00 | 33.64 | N |
| ATOM | 3884 | CA | TRP | B | 169 | −31.837 | 20.617 | 19.295 | 1.00 | 37.05 | C |
| ATOM | 3885 | CB | TRP | B | 169 | −32.364 | 21.138 | 17.959 | 1.00 | 34.37 | C |
| ATOM | 3886 | CG | TRP | B | 169 | −31.320 | 21.294 | 16.898 | 1.00 | 36.96 | C |
| ATOM | 3887 | CD1 | TRP | B | 169 | −30.879 | 20.331 | 16.042 | 1.00 | 38.36 | C |
| ATOM | 3888 | NE1 | TRP | B | 169 | −29.927 | 20.843 | 15.199 | 1.00 | 37.30 | N |
| ATOM | 3889 | CE2 | TRP | B | 169 | −29.739 | 22.166 | 15.496 | 1.00 | 38.21 | C |
| ATOM | 3890 | CD2 | TRP | B | 169 | −30.603 | 22.489 | 16.561 | 1.00 | 38.14 | C |
| ATOM | 3891 | CE3 | TRP | B | 169 | −30.602 | 23.796 | 17.057 | 1.00 | 40.43 | C |
| ATOM | 3892 | CZ3 | TRP | B | 169 | −29.748 | 24.721 | 16.481 | 1.00 | 37.89 | C |
| ATOM | 3893 | CH2 | TRP | B | 169 | −28.903 | 24.365 | 15.425 | 1.00 | 39.23 | C |
| ATOM | 3894 | CZ2 | TRP | B | 169 | −28.885 | 23.096 | 14.921 | 1.00 | 42.18 | C |
| ATOM | 3895 | C | TRP | B | 169 | −30.825 | 21.591 | 19.879 | 1.00 | 38.31 | C |
| ATOM | 3896 | O | TRP | B | 169 | −29.650 | 21.317 | 19.923 | 1.00 | 35.86 | O |
| ATOM | 3897 | N | PHE | B | 170 | −31.301 | 22.683 | 20.433 | 1.00 | 33.07 | N |
| ATOM | 3898 | CA | PHE | B | 170 | −30.408 | 23.657 | 21.034 | 1.00 | 35.44 | C |
| ATOM | 3899 | CB | PHE | B | 170 | −31.182 | 24.866 | 21.555 | 1.00 | 28.47 | C |
| ATOM | 3900 | CG | PHE | B | 170 | −31.734 | 25.738 | 20.466 | 1.00 | 25.70 | C |
| ATOM | 3901 | CD1 | PHE | B | 170 | −30.941 | 26.692 | 19.856 | 1.00 | 28.27 | C |
| ATOM | 3902 | CE1 | PHE | B | 170 | −31.447 | 27.495 | 18.848 | 1.00 | 33.01 | C |
| ATOM | 3903 | CZ | PHE | B | 170 | −32.756 | 27.349 | 18.440 | 1.00 | 34.82 | C |
| ATOM | 3904 | CE2 | PHE | B | 170 | −33.556 | 26.400 | 19.039 | 1.00 | 38.11 | C |
| ATOM | 3905 | CD2 | PHE | B | 170 | −33.043 | 25.599 | 20.046 | 1.00 | 37.10 | C |
| ATOM | 3906 | C | PHE | B | 170 | −29.516 | 23.029 | 22.125 | 1.00 | 37.76 | C |
| ATOM | 3907 | O | PHE | B | 170 | −28.344 | 23.280 | 22.213 | 1.00 | 41.58 | O |
| ATOM | 3908 | N | VAL | B | 171 | −30.074 | 22.151 | 22.912 | 1.00 | 35.87 | N |
| ATOM | 3909 | CA | VAL | B | 171 | −29.302 | 21.424 | 23.897 | 1.00 | 32.89 | C |
| ATOM | 3910 | CB | VAL | B | 171 | −30.221 | 20.619 | 24.833 | 1.00 | 32.50 | C |
| ATOM | 3911 | CG1 | VAL | B | 171 | −29.419 | 20.014 | 25.972 | 1.00 | 32.84 | C |
| ATOM | 3912 | CG2 | VAL | B | 171 | −31.330 | 21.507 | 25.374 | 1.00 | 31.05 | C |
| ATOM | 3913 | C | VAL | B | 171 | −28.288 | 20.474 | 23.245 | 1.00 | 36.24 | C |
| ATOM | 3914 | O | VAL | B | 171 | −27.168 | 20.319 | 23.672 | 1.00 | 37.60 | O |
| ATOM | 3915 | N | VAL | B | 172 | −28.722 | 19.788 | 22.215 | 1.00 | 38.30 | N |
| ATOM | 3916 | CA | VAL | B | 172 | −27.904 | 18.804 | 21.520 | 1.00 | 39.36 | C |
| ATOM | 3917 | CB | VAL | B | 172 | −28.720 | 18.110 | 20.381 | 1.00 | 36.28 | C |
| ATOM | 3918 | CG1 | VAL | B | 172 | −27.836 | 17.248 | 19.487 | 1.00 | 42.54 | C |
| ATOM | 3919 | CG2 | VAL | B | 172 | −29.835 | 17.271 | 20.955 | 1.00 | 35.84 | C |
| ATOM | 3920 | C | VAL | B | 172 | −26.672 | 19.433 | 20.917 | 1.00 | 39.79 | C |
| ATOM | 3921 | O | VAL | B | 172 | −25.658 | 18.809 | 20.794 | 1.00 | 41.68 | O |
| ATOM | 3922 | N | GLN | B | 173 | −26.754 | 20.696 | 20.604 | 1.00 | 41.94 | N |
| ATOM | 3923 | CA | GLN | B | 173 | −25.643 | 21.442 | 20.070 | 1.00 | 41.76 | C |
| ATOM | 3924 | CB | GLN | B | 173 | −26.090 | 22.834 | 19.625 | 1.00 | 42.89 | C |
| ATOM | 3925 | CG | GLN | B | 173 | −27.011 | 22.797 | 18.419 | 1.00 | 41.83 | C |
| ATOM | 3926 | CD | GLN | B | 173 | −26.426 | 21.978 | 17.285 | 1.00 | 43.44 | C |
| ATOM | 3927 | OE1 | GLN | B | 173 | −27.035 | 21.014 | 16.819 | 1.00 | 38.35 | O |
| ATOM | 3928 | NE2 | GLN | B | 173 | −25.233 | 22.358 | 16.836 | 1.00 | 55.09 | N |
| ATOM | 3929 | C | GLN | B | 173 | −24.494 | 21.533 | 21.044 | 1.00 | 44.01 | C |
| ATOM | 3930 | O | GLN | B | 173 | −23.356 | 21.432 | 20.660 | 1.00 | 53.00 | O |
| ATOM | 3931 | N | LEU | B | 174 | −24.780 | 21.709 | 22.319 | 1.00 | 44.73 | N |
| ATOM | 3932 | CA | LEU | B | 174 | −23.744 | 21.602 | 23.343 | 1.00 | 43.83 | C |
| ATOM | 3933 | CB | LEU | B | 174 | −24.356 | 21.718 | 24.738 | 1.00 | 39.49 | C |
| ATOM | 3934 | CG | LEU | B | 174 | −25.157 | 22.983 | 25.038 | 1.00 | 40.60 | C |
| ATOM | 3935 | CD1 | LEU | B | 174 | −25.791 | 22.873 | 26.413 | 1.00 | 45.62 | C |
| ATOM | 3936 | CD2 | LEU | B | 174 | −24.278 | 24.224 | 24.945 | 1.00 | 32.79 | C |
| ATOM | 3937 | C | LEU | B | 174 | −22.966 | 20.282 | 23.244 | 1.00 | 43.19 | C |
| ATOM | 3938 | O | LEU | B | 174 | −21.756 | 20.259 | 23.295 | 1.00 | 48.30 | O |
| ATOM | 3939 | N | THR | B | 175 | −23.671 | 19.188 | 23.026 | 1.00 | 45.62 | N |
| ATOM | 3940 | CA | THR | B | 175 | −23.008 | 17.908 | 22.844 | 1.00 | 43.23 | C |
| ATOM | 3941 | CB | THR | B | 175 | −24.013 | 16.731 | 22.938 | 1.00 | 42.63 | C |
| ATOM | 3942 | OG1 | THR | B | 175 | −24.868 | 16.718 | 21.786 | 1.00 | 36.15 | O |
| ATOM | 3943 | CG2 | THR | B | 175 | −24.859 | 16.843 | 24.195 | 1.00 | 47.74 | C |
| ATOM | 3944 | C | THR | B | 175 | −22.242 | 17.752 | 21.514 | 1.00 | 39.55 | C |
| ATOM | 3945 | O | THR | B | 175 | −21.546 | 16.785 | 21.321 | 1.00 | 40.80 | O |
| ATOM | 3946 | N | MET | B | 176 | −22.383 | 18.676 | 20.577 | 1.00 | 44.90 | N |
| ATOM | 3947 | CA | MET | B | 176 | −21.636 | 18.598 | 19.327 | 1.00 | 43.65 | C |
| ATOM | 3948 | CB | MET | B | 176 | −22.209 | 19.562 | 18.282 | 1.00 | 49.51 | C |
| ATOM | 3949 | CG | MET | B | 176 | −23.525 | 19.121 | 17.644 | 1.00 | 49.44 | C |
| ATOM | 3950 | SD | MET | B | 176 | −23.387 | 17.631 | 16.627 | 1.00 | 59.32 | S |
| ATOM | 3951 | CE | MET | B | 176 | −24.894 | 17.724 | 15.662 | 1.00 | 48.90 | C |
| ATOM | 3952 | C | MET | B | 176 | −20.138 | 18.852 | 19.502 | 1.00 | 52.28 | C |
| ATOM | 3953 | O | MET | B | 176 | −19.353 | 18.549 | 18.615 | 1.00 | 54.49 | O |
| ATOM | 3954 | O | SER | B | 177 | −17.833 | 17.544 | 21.347 | 1.00 | 60.09 | O |
| ATOM | 3955 | N | SER | B | 177 | −19.751 | 19.505 | 20.617 | 1.00 | 56.60 | N |
| ATOM | 3956 | CA | SER | B | 177 | −18.334 | 19.826 | 20.864 | 1.00 | 59.33 | C |
| ATOM | 3957 | C | SER | B | 177 | −17.442 | 18.565 | 20.811 | 1.00 | 60.52 | C |
| ATOM | 3958 | CB | SER | B | 177 | −18.160 | 20.499 | 22.230 | 1.00 | 59.13 | C |
| ATOM | 3959 | OG | SER | B | 177 | −19.010 | 21.624 | 22.373 | 1.00 | 69.30 | O |
| ATOM | 3960 | O | PRO | B | 178 | −14.939 | 15.552 | 21.516 | 1.00 | 51.86 | O |
| ATOM | 3961 | N | PRO | B | 178 | −16.243 | 18.543 | 20.164 | 1.00 | 65.51 | N |
| ATOM | 3962 | CA | PRO | B | 178 | −15.381 | 17.364 | 20.009 | 1.00 | 63.26 | C |
| ATOM | 3963 | C | PRO | B | 178 | −14.969 | 16.766 | 21.366 | 1.00 | 56.14 | C |
| ATOM | 3964 | CB | PRO | B | 178 | −14.157 | 17.906 | 19.257 | 1.00 | 57.38 | C |
| ATOM | 3965 | CG | PRO | B | 178 | −14.250 | 19.392 | 19.339 | 1.00 | 62.41 | C |
| ATOM | 3966 | CD | PRO | B | 178 | −15.705 | 19.708 | 19.447 | 1.00 | 62.79 | C |
| ATOM | 3967 | O | GLU | B | 179 | −15.339 | 15.389 | 25.005 | 1.00 | 54.37 | O |
| ATOM | 3968 | N | GLU | B | 179 | −14.662 | 17.604 | 22.344 | 1.00 | 56.78 | N |
| ATOM | 3969 | CA | GLU | B | 179 | −14.289 | 17.120 | 23.661 | 1.00 | 60.98 | C |
| ATOM | 3970 | C | GLU | B | 179 | −15.477 | 16.433 | 24.384 | 1.00 | 56.32 | C |
| ATOM | 3971 | CB | GLU | B | 179 | −13.706 | 18.251 | 24.510 | 1.00 | 71.15 | C |
| ATOM | 3972 | CG | GLU | B | 179 | −12.394 | 18.799 | 23.968 | 1.00 | 68.19 | C |
| ATOM | 3973 | CD | GLU | B | 179 | −11.884 | 19.996 | 24.748 | 1.00 | 91.88 | C |
| ATOM | 3974 | OE1 | GLU | B | 179 | −12.641 | 20.537 | 25.582 | 1.00 | 95.79 | O |
| ATOM | 3975 | OE2 | GLU | B | 179 | −10.720 | 20.396 | 24.523 | 1.00 | 103.73 | O |
| ATOM | 3976 | N | VAL | B | 180 | −16.671 | 17.014 | 24.224 | 1.00 | 65.42 | N |
| ATOM | 3977 | CA | VAL | B | 180 | −17.915 | 16.450 | 24.772 | 1.00 | 60.77 | C |
| ATOM | 3978 | CB | VAL | B | 180 | −19.073 | 17.477 | 24.676 | 1.00 | 55.70 | C |
| ATOM | 3979 | CG1 | VAL | B | 180 | −20.372 | 16.899 | 25.223 | 1.00 | 52.89 | C |
| ATOM | 3980 | CG2 | VAL | B | 180 | −18.708 | 18.756 | 25.412 | 1.00 | 61.93 | C |
| ATOM | 3981 | C | VAL | B | 180 | −18.333 | 15.157 | 24.077 | 1.00 | 54.49 | C |
| ATOM | 3982 | O | VAL | B | 180 | −18.843 | 14.261 | 24.700 | 1.00 | 48.37 | O |
| ATOM | 3983 | N | HIS | B | 181 | −18.157 | 15.095 | 22.764 | 1.00 | 50.30 | N |
| ATOM | 3984 | CA | HIS | B | 181 | −18.533 | 13.932 | 21.998 | 1.00 | 43.91 | C |
| ATOM | 3985 | CB | HIS | B | 181 | −18.311 | 14.264 | 20.513 | 1.00 | 47.48 | C |
| ATOM | 3986 | CG | HIS | B | 181 | −19.119 | 13.432 | 19.565 | 1.00 | 51.88 | C |
| ATOM | 3987 | ND1 | HIS | B | 181 | −20.496 | 13.455 | 19.550 | 1.00 | 59.90 | N |
| ATOM | 3988 | CE1 | HIS | B | 181 | −20.937 | 12.636 | 18.611 | 1.00 | 64.19 | C |
| ATOM | 3989 | NE2 | HIS | B | 181 | −19.895 | 12.090 | 18.012 | 1.00 | 61.19 | N |
| ATOM | 3990 | CD2 | HIS | B | 181 | −18.744 | 12.577 | 18.585 | 1.00 | 49.14 | C |
| ATOM | 3991 | C | HIS | B | 181 | −17.702 | 12.736 | 22.354 | 1.00 | 48.47 | C |
| ATOM | 3992 | O | HIS | B | 181 | −18.194 | 11.645 | 22.498 | 1.00 | 48.18 | O |
| ATOM | 3993 | N | GLN | B | 182 | −16.403 | 12.952 | 22.471 | 1.00 | 55.57 | N |
| ATOM | 3994 | CA | GLN | B | 182 | −15.520 | 11.890 | 22.872 | 1.00 | 54.72 | C |
| ATOM | 3995 | CB | GLN | B | 182 | −14.057 | 12.305 | 22.700 | 1.00 | 51.78 | C |
| ATOM | 3996 | CG | GLN | B | 182 | −13.606 | 12.405 | 21.254 | 1.00 | 50.57 | C |
| ATOM | 3997 | CD | GLN | B | 182 | −12.102 | 12.529 | 21.130 | 1.00 | 51.12 | C |
| ATOM | 3998 | OE1 | GLN | B | 182 | −11.369 | 12.338 | 22.102 | 1.00 | 48.84 | O |
| ATOM | 3999 | NE2 | GLN | B | 182 | −11.632 | 12.858 | 19.933 | 1.00 | 50.91 | N |
| ATOM | 4000 | C | GLN | B | 182 | −15.767 | 11.436 | 24.298 | 1.00 | 51.16 | C |
| ATOM | 4001 | O | GLN | B | 182 | −16.034 | 10.271 | 24.524 | 1.00 | 50.74 | O |
| ATOM | 4002 | N | SER | B | 183 | −15.818 | 12.372 | 25.260 | 1.00 | 52.38 | N |
| ATOM | 4003 | CA | SER | B | 183 | −16.194 | 11.951 | 26.596 | 1.00 | 57.96 | C |
| ATOM | 4004 | CB | SER | B | 183 | −16.008 | 13.098 | 27.593 | 1.00 | 61.46 | C |
| ATOM | 4005 | OG | SER | B | 183 | −16.971 | 14.122 | 27.381 | 1.00 | 54.69 | O |
| ATOM | 4006 | C | SER | B | 183 | −17.639 | 11.577 | 26.568 | 1.00 | 57.11 | C |
| ATOM | 4007 | O | SER | B | 183 | −18.475 | 12.393 | 26.278 | 1.00 | 63.37 | O |
| ATOM | 4008 | N | ASN | B | 184 | −17.950 | 10.365 | 26.865 | 1.00 | 53.52 | N |
| ATOM | 4009 | CA | ASN | B | 184 | −19.290 | 9.947 | 26.709 | 1.00 | 48.83 | C |
| ATOM | 4010 | CB | ASN | B | 184 | −19.261 | 8.522 | 26.158 | 1.00 | 49.76 | C |
| ATOM | 4011 | CG | ASN | B | 184 | −20.618 | 8.001 | 25.780 | 1.00 | 50.65 | C |
| ATOM | 4012 | OD1 | ASN | B | 184 | −21.386 | 8.673 | 25.103 | 1.00 | 63.29 | O |
| ATOM | 4013 | ND2 | ASN | B | 184 | −20.923 | 6.782 | 26.218 | 1.00 | 46.97 | N |
| ATOM | 4014 | C | ASN | B | 184 | −19.815 | 9.944 | 28.081 | 1.00 | 48.30 | C |
| ATOM | 4015 | O | ASN | B | 184 | −19.167 | 9.435 | 28.971 | 1.00 | 65.95 | O |
| ATOM | 4016 | N | VAL | B | 185 | −20.863 | 10.698 | 28.303 | 1.00 | 44.16 | N |
| ATOM | 4017 | CA | VAL | B | 185 | −21.244 | 10.953 | 29.650 | 1.00 | 51.77 | C |
| ATOM | 4018 | CB | VAL | B | 185 | −20.757 | 12.371 | 30.127 | 1.00 | 40.37 | C |
| ATOM | 4019 | CG1 | VAL | B | 185 | −20.967 | 12.555 | 31.628 | 1.00 | 42.02 | C |
| ATOM | 4020 | CG2 | VAL | B | 185 | −19.296 | 12.626 | 29.774 | 1.00 | 44.17 | C |
| ATOM | 4021 | C | VAL | B | 185 | −22.719 | 10.960 | 29.678 | 1.00 | 51.57 | C |
| ATOM | 4022 | O | VAL | B | 185 | −23.353 | 11.135 | 28.663 | 1.00 | 54.29 | O |
| ATOM | 4023 | O | GLY | B | 186 | −26.423 | 11.606 | 29.855 | 1.00 | 47.71 | O |
| ATOM | 4024 | N | GLY | B | 186 | −23.269 | 10.733 | 30.855 | 1.00 | 47.42 | N |
| ATOM | 4025 | CA | GLY | B | 186 | −24.672 | 10.632 | 30.993 | 1.00 | 43.91 | C |
| ATOM | 4026 | C | GLY | B | 186 | −25.410 | 11.787 | 30.479 | 1.00 | 44.73 | C |
| ATOM | 4027 | O | TRP | B | 187 | −26.696 | 14.596 | 28.162 | 1.00 | 44.32 | O |
| ATOM | 4028 | N | TRP | B | 187 | −24.937 | 12.989 | 30.717 | 1.00 | 38.28 | N |
| ATOM | 4029 | CA | TRP | B | 187 | −25.661 | 14.133 | 30.275 | 1.00 | 43.77 | C |
| ATOM | 4030 | C | TRP | B | 187 | −25.705 | 14.221 | 28.745 | 1.00 | 43.30 | C |
| ATOM | 4031 | CB | TRP | B | 187 | −25.162 | 15.424 | 30.959 | 1.00 | 44.19 | C |
| ATOM | 4032 | CG | TRP | B | 187 | −23.926 | 16.054 | 30.387 | 1.00 | 56.19 | C |
| ATOM | 4033 | CD1 | TRP | B | 187 | −22.649 | 15.917 | 30.844 | 1.00 | 50.22 | C |
| ATOM | 4034 | CD2 | TRP | B | 187 | −23.862 | 16.953 | 29.271 | 1.00 | 59.28 | C |
| ATOM | 4035 | NE1 | TRP | B | 187 | −21.789 | 16.659 | 30.067 | 1.00 | 51.37 | N |
| ATOM | 4036 | CE2 | TRP | B | 187 | −22.509 | 17.302 | 29.094 | 1.00 | 57.61 | C |
| ATOM | 4037 | CE3 | TRP | B | 187 | −24.815 | 17.484 | 28.394 | 1.00 | 53.14 | C |
| ATOM | 4038 | CZ2 | TRP | B | 187 | −22.085 | 18.158 | 28.079 | 1.00 | 66.23 | C |
| ATOM | 4039 | CZ3 | TRP | B | 187 | −24.394 | 18.335 | 27.385 | 1.00 | 53.07 | C |
| ATOM | 4040 | CH2 | TRP | B | 187 | −23.040 | 18.661 | 27.235 | 1.00 | 62.56 | C |
| ATOM | 4041 | O | CYS | B | 188 | −26.333 | 12.973 | 25.116 | 1.00 | 34.39 | O |
| ATOM | 4042 | N | CYS | B | 188 | −24.639 | 13.794 | 28.093 | 1.00 | 47.23 | N |
| ATOM | 4043 | CA | CYS | B | 188 | −24.597 | 13.668 | 26.636 | 1.00 | 52.38 | C |
| ATOM | 4044 | C | CYS | B | 188 | −25.632 | 12.695 | 26.051 | 1.00 | 40.35 | C |
| ATOM | 4045 | CB | CYS | B | 188 | −23.194 | 13.229 | 26.222 | 1.00 | 51.78 | C |
| ATOM | 4046 | SG | CYS | B | 188 | −22.705 | 13.819 | 24.615 | 1.00 | 75.85 | S |
| ATOM | 4047 | N | ARG | B | 189 | −25.638 | 11.517 | 26.624 | 1.00 | 43.02 | N |
| ATOM | 4048 | CA | ARG | B | 189 | −26.459 | 10.404 | 26.230 | 1.00 | 41.04 | C |
| ATOM | 4049 | CB | ARG | B | 189 | −25.952 | 9.101 | 26.852 | 1.00 | 42.74 | C |
| ATOM | 4050 | CG | ARG | B | 189 | −24.714 | 8.557 | 26.154 | 1.00 | 39.43 | C |
| ATOM | 4051 | CD | ARG | B | 189 | −24.124 | 7.365 | 26.882 | 1.00 | 40.26 | C |
| ATOM | 4052 | NE | ARG | B | 189 | −23.629 | 7.714 | 28.210 | 1.00 | 45.18 | N |
| ATOM | 4053 | CZ | ARG | B | 189 | −22.901 | 6.902 | 28.968 | 1.00 | 44.22 | C |
| ATOM | 4054 | NH1 | ARG | B | 189 | −22.583 | 5.693 | 28.524 | 1.00 | 43.72 | N |
| ATOM | 4055 | NH2 | ARG | B | 189 | −22.489 | 7.297 | 30.166 | 1.00 | 41.70 | N |
| ATOM | 4056 | C | ARG | B | 189 | −27.913 | 10.619 | 26.510 | 1.00 | 43.04 | C |
| ATOM | 4057 | O | ARG | B | 189 | −28.759 | 10.304 | 25.706 | 1.00 | 38.68 | O |
| ATOM | 4058 | N | LEU | B | 190 | −28.203 | 11.213 | 27.652 | 1.00 | 43.02 | N |
| ATOM | 4059 | CA | LEU | B | 190 | −29.548 | 11.579 | 28.022 | 1.00 | 38.50 | C |
| ATOM | 4060 | CB | LEU | B | 190 | −29.596 | 12.220 | 29.409 | 1.00 | 32.71 | C |
| ATOM | 4061 | CG | LEU | B | 190 | −31.019 | 12.339 | 29.966 | 1.00 | 35.01 | C |
| ATOM | 4062 | CD1 | LEU | B | 190 | −31.371 | 11.100 | 30.770 | 1.00 | 39.24 | C |
| ATOM | 4063 | CD2 | LEU | B | 190 | −31.219 | 13.604 | 30.793 | 1.00 | 28.87 | C |
| ATOM | 4064 | C | LEU | B | 190 | −30.129 | 12.530 | 27.001 | 1.00 | 39.16 | C |
| ATOM | 4065 | O | LEU | B | 190 | −31.236 | 12.388 | 26.565 | 1.00 | 35.95 | O |
| ATOM | 4066 | N | VAL | B | 191 | −29.333 | 13.505 | 26.619 | 1.00 | 34.23 | N |
| ATOM | 4067 | CA | VAL | B | 191 | −29.718 | 14.495 | 25.656 | 1.00 | 29.16 | C |
| ATOM | 4068 | CB | VAL | B | 191 | −28.682 | 15.636 | 25.559 | 1.00 | 30.94 | C |
| ATOM | 4069 | CG1 | VAL | B | 191 | −28.932 | 16.488 | 24.328 | 1.00 | 33.45 | C |
| ATOM | 4070 | CG2 | VAL | B | 191 | −28.736 | 16.497 | 26.806 | 1.00 | 27.25 | C |
| ATOM | 4071 | C | VAL | B | 191 | −29.981 | 13.891 | 24.284 | 1.00 | 32.58 | C |
| ATOM | 4072 | O | VAL | B | 191 | −31.022 | 14.106 | 23.724 | 1.00 | 37.99 | O |
| ATOM | 4073 | N | THR | B | 192 | −29.086 | 13.043 | 23.802 | 1.00 | 31.92 | N |
| ATOM | 4074 | CA | THR | B | 192 | −29.292 | 12.278 | 22.564 | 1.00 | 30.53 | C |
| ATOM | 4075 | CB | THR | B | 192 | −28.077 | 11.383 | 22.215 | 1.00 | 31.56 | C |
| ATOM | 4076 | OG1 | THR | B | 192 | −26.903 | 12.192 | 22.072 | 1.00 | 38.20 | O |
| ATOM | 4077 | CG2 | THR | B | 192 | −28.316 | 10.620 | 20.914 | 1.00 | 27.04 | C |
| ATOM | 4078 | C | THR | B | 192 | −30.538 | 11.421 | 22.603 | 1.00 | 31.56 | C |
| ATOM | 4079 | O | THR | B | 192 | −31.252 | 11.357 | 21.648 | 1.00 | 35.32 | O |
| ATOM | 4080 | N | ALA | B | 193 | −30.784 | 10.754 | 23.720 | 1.00 | 33.58 | N |
| ATOM | 4081 | CA | ALA | B | 193 | −31.962 | 9.908 | 23.913 | 1.00 | 34.30 | C |
| ATOM | 4082 | CB | ALA | B | 193 | −31.873 | 9.163 | 25.242 | 1.00 | 30.81 | C |
| ATOM | 4083 | C | ALA | B | 193 | −33.266 | 10.709 | 23.834 | 1.00 | 39.14 | C |
| ATOM | 4084 | O | ALA | B | 193 | −34.154 | 10.397 | 23.075 | 1.00 | 34.05 | O |
| ATOM | 4085 | N | ALA | B | 194 | −33.307 | 11.813 | 24.572 | 1.00 | 35.31 | N |
| ATOM | 4086 | CA | ALA | B | 194 | −34.418 | 12.752 | 24.579 | 1.00 | 32.60 | C |
| ATOM | 4087 | CB | ALA | B | 194 | −34.152 | 13.862 | 25.588 | 1.00 | 34.15 | C |
| ATOM | 4088 | C | ALA | B | 194 | −34.686 | 13.343 | 23.212 | 1.00 | 31.49 | C |
| ATOM | 4089 | O | ALA | B | 194 | −35.798 | 13.429 | 22.773 | 1.00 | 36.42 | O |
| ATOM | 4090 | N | TYR | B | 195 | −33.640 | 13.710 | 22.511 | 1.00 | 31.81 | N |
| ATOM | 4091 | CA | TYR | B | 195 | −33.751 | 14.249 | 21.172 | 1.00 | 35.15 | C |
| ATOM | 4092 | CB | TYR | B | 195 | −32.365 | 14.649 | 20.656 | 1.00 | 33.37 | C |
| ATOM | 4093 | CG | TYR | B | 195 | −32.379 | 15.359 | 19.324 | 1.00 | 32.11 | C |
| ATOM | 4094 | CD1 | TYR | B | 195 | −32.952 | 16.618 | 19.193 | 1.00 | 35.64 | C |
| ATOM | 4095 | CE1 | TYR | B | 195 | −32.966 | 17.274 | 17.979 | 1.00 | 35.99 | C |
| ATOM | 4096 | CZ | TYR | B | 195 | −32.400 | 16.674 | 16.880 | 1.00 | 37.57 | C |
| ATOM | 4097 | OH | TYR | B | 195 | −32.416 | 17.327 | 15.671 | 1.00 | 44.26 | O |
| ATOM | 4098 | CE2 | TYR | B | 195 | −31.823 | 15.426 | 16.983 | 1.00 | 40.46 | C |
| ATOM | 4099 | CD2 | TYR | B | 195 | −31.812 | 14.778 | 18.201 | 1.00 | 32.30 | C |
| ATOM | 4100 | C | TYR | B | 195 | −34.399 | 13.265 | 20.225 | 1.00 | 31.90 | C |
| ATOM | 4101 | O | TYR | B | 195 | −35.353 | 13.562 | 19.563 | 1.00 | 27.59 | O |
| ATOM | 4102 | N | ASN | B | 196 | −33.862 | 12.073 | 20.204 | 1.00 | 36.11 | N |
| ATOM | 4103 | CA | ASN | B | 196 | −34.348 | 10.974 | 19.396 | 1.00 | 34.85 | C |
| ATOM | 4104 | CB | ASN | B | 196 | −33.397 | 9.772 | 19.473 | 1.00 | 34.66 | C |
| ATOM | 4105 | CG | ASN | B | 196 | −32.183 | 9.930 | 18.582 | 1.00 | 30.56 | C |
| ATOM | 4106 | OD1 | ASN | B | 196 | −32.225 | 10.638 | 17.576 | 1.00 | 36.14 | O |
| ATOM | 4107 | ND2 | ASN | B | 196 | −31.092 | 9.268 | 18.945 | 1.00 | 31.83 | N |
| ATOM | 4108 | C | ASN | B | 196 | −35.752 | 10.536 | 19.793 | 1.00 | 35.53 | C |
| ATOM | 4109 | O | ASN | B | 196 | −36.504 | 10.132 | 18.949 | 1.00 | 38.37 | O |
| ATOM | 4110 | N | TYR | B | 197 | −36.115 | 10.649 | 21.077 | 1.00 | 39.00 | N |
| ATOM | 4111 | CA | TYR | B | 197 | −37.482 | 10.402 | 21.541 | 1.00 | 39.99 | C |
| ATOM | 4112 | CB | TYR | B | 197 | −37.559 | 10.531 | 23.066 | 1.00 | 35.01 | C |
| ATOM | 4113 | CG | TYR | B | 197 | −38.966 | 10.467 | 23.621 | 1.00 | 40.25 | C |
| ATOM | 4114 | CD1 | TYR | B | 197 | −39.605 | 9.246 | 23.820 | 1.00 | 39.85 | C |
| ATOM | 4115 | CE1 | TYR | B | 197 | −40.896 | 9.188 | 24.325 | 1.00 | 36.84 | C |
| ATOM | 4116 | CZ | TYR | B | 197 | −41.555 | 10.356 | 24.638 | 1.00 | 36.61 | C |
| ATOM | 4117 | OH | TYR | B | 197 | −42.831 | 10.312 | 25.141 | 1.00 | 47.96 | O |
| ATOM | 4118 | CE2 | TYR | B | 197 | −40.943 | 11.579 | 24.452 | 1.00 | 36.34 | C |
| ATOM | 4119 | CD2 | TYR | B | 197 | −39.658 | 11.629 | 23.948 | 1.00 | 40.42 | C |
| ATOM | 4120 | C | TYR | B | 197 | −38.460 | 11.340 | 20.894 | 1.00 | 35.88 | C |
| ATOM | 4121 | O | TYR | B | 197 | −39.434 | 10.922 | 20.320 | 1.00 | 34.27 | O |
| ATOM | 4122 | N | PHE | B | 198 | −38.107 | 12.598 | 20.873 | 1.00 | 35.70 | N |
| ATOM | 4123 | CA | PHE | B | 198 | −38.899 | 13.628 | 20.254 | 1.00 | 36.61 | C |
| ATOM | 4124 | CB | PHE | B | 198 | −38.383 | 15.026 | 20.597 | 1.00 | 37.54 | C |
| ATOM | 4125 | CG | PHE | B | 198 | −38.610 | 15.390 | 22.037 | 1.00 | 35.52 | C |
| ATOM | 4126 | CD1 | PHE | B | 198 | −39.811 | 15.080 | 22.655 | 1.00 | 30.39 | C |
| ATOM | 4127 | CE1 | PHE | B | 198 | −40.022 | 15.390 | 23.978 | 1.00 | 30.99 | C |
| ATOM | 4128 | CZ | PHE | B | 198 | −39.028 | 16.004 | 24.710 | 1.00 | 30.42 | C |
| ATOM | 4129 | CE2 | PHE | B | 198 | −37.828 | 16.306 | 24.114 | 1.00 | 28.96 | C |
| ATOM | 4130 | CD2 | PHE | B | 198 | −37.618 | 15.993 | 22.785 | 1.00 | 33.55 | C |
| ATOM | 4131 | C | PHE | B | 198 | −39.133 | 13.382 | 18.771 | 1.00 | 37.21 | C |
| ATOM | 4132 | O | PHE | B | 198 | −40.217 | 13.541 | 18.289 | 1.00 | 38.41 | O |
| ATOM | 4133 | N | HIS | B | 199 | −38.128 | 12.914 | 18.059 | 1.00 | 38.44 | N |
| ATOM | 4134 | CA | HIS | B | 199 | −38.244 | 12.590 | 16.645 | 1.00 | 38.01 | C |
| ATOM | 4135 | CB | HIS | B | 199 | −36.883 | 12.092 | 16.152 | 1.00 | 39.21 | C |
| ATOM | 4136 | CG | HIS | B | 199 | −36.642 | 12.317 | 14.695 | 1.00 | 40.96 | C |
| ATOM | 4137 | ND1 | HIS | B | 199 | −35.665 | 11.645 | 13.993 | 1.00 | 45.11 | N |
| ATOM | 4138 | CE1 | HIS | B | 199 | −35.676 | 12.048 | 12.735 | 1.00 | 46.16 | C |
| ATOM | 4139 | NE2 | HIS | B | 199 | −36.622 | 12.958 | 12.597 | 1.00 | 43.93 | N |
| ATOM | 4140 | CD2 | HIS | B | 199 | −37.240 | 13.146 | 13.809 | 1.00 | 43.23 | C |
| ATOM | 4141 | C | HIS | B | 199 | −39.296 | 11.504 | 16.396 | 1.00 | 36.86 | C |
| ATOM | 4142 | O | HIS | B | 199 | −40.150 | 11.611 | 15.538 | 1.00 | 40.80 | O |
| ATOM | 4143 | N | VAL | B | 200 | −39.235 | 10.455 | 17.212 | 1.00 | 34.02 | N |
| ATOM | 4144 | CA | VAL | B | 200 | −40.177 | 9.355 | 17.171 | 1.00 | 37.59 | C |
| ATOM | 4145 | CB | VAL | B | 200 | −39.786 | 8.178 | 18.077 | 1.00 | 37.17 | C |
| ATOM | 4146 | CG1 | VAL | B | 200 | −40.576 | 6.949 | 17.673 | 1.00 | 34.54 | C |
| ATOM | 4147 | CG2 | VAL | B | 200 | −38.314 | 7.880 | 17.933 | 1.00 | 36.68 | C |
| ATOM | 4148 | C | VAL | B | 200 | −41.563 | 9.852 | 17.471 | 1.00 | 38.55 | C |
| ATOM | 4149 | O | VAL | B | 200 | −42.506 | 9.537 | 16.795 | 1.00 | 38.83 | O |
| ATOM | 4150 | N | THR | B | 201 | −41.625 | 10.726 | 18.449 | 1.00 | 39.95 | N |
| ATOM | 4151 | CA | THR | B | 201 | −42.823 | 11.435 | 18.829 | 1.00 | 38.82 | C |
| ATOM | 4152 | CB | THR | B | 201 | −42.552 | 12.393 | 20.014 | 1.00 | 36.28 | C |
| ATOM | 4153 | OG1 | THR | B | 201 | −42.173 | 11.635 | 21.169 | 1.00 | 49.51 | O |
| ATOM | 4154 | CG2 | THR | B | 201 | −43.783 | 13.207 | 20.344 | 1.00 | 33.58 | C |
| ATOM | 4155 | C | THR | B | 201 | −43.415 | 12.229 | 17.674 | 1.00 | 38.83 | C |
| ATOM | 4156 | O | THR | B | 201 | −44.599 | 12.216 | 17.479 | 1.00 | 38.33 | O |
| ATOM | 4157 | N | ASN | B | 202 | −42.592 | 12.927 | 16.907 | 1.00 | 40.91 | N |
| ATOM | 4158 | CA | ASN | B | 202 | −43.070 | 13.682 | 15.762 | 1.00 | 36.14 | C |
| ATOM | 4159 | CB | ASN | B | 202 | −41.905 | 14.437 | 15.112 | 1.00 | 33.23 | C |
| ATOM | 4160 | CG | ASN | B | 202 | −41.423 | 15.615 | 15.944 | 1.00 | 35.68 | C |
| ATOM | 4161 | OD1 | ASN | B | 202 | −41.812 | 15.782 | 17.101 | 1.00 | 37.12 | O |
| ATOM | 4162 | ND2 | ASN | B | 202 | −40.565 | 16.439 | 15.352 | 1.00 | 35.44 | N |
| ATOM | 4163 | C | ASN | B | 202 | −43.722 | 12.813 | 14.716 | 1.00 | 37.65 | C |
| ATOM | 4164 | O | ASN | B | 202 | −44.791 | 13.102 | 14.266 | 1.00 | 40.38 | O |
| ATOM | 4165 | N | PHE | B | 203 | −43.089 | 11.725 | 14.366 | 1.00 | 36.13 | N |
| ATOM | 4166 | CA | PHE | B | 203 | −43.673 | 10.760 | 13.447 | 1.00 | 39.48 | C |
| ATOM | 4167 | CB | PHE | B | 203 | −42.679 | 9.667 | 13.075 | 1.00 | 40.44 | C |
| ATOM | 4168 | CG | PHE | B | 203 | −41.803 | 10.027 | 11.913 | 1.00 | 46.41 | C |
| ATOM | 4169 | CD2 | PHE | B | 203 | −41.826 | 9.275 | 10.753 | 1.00 | 45.96 | C |
| ATOM | 4170 | CE2 | PHE | B | 203 | −41.011 | 9.602 | 9.684 | 1.00 | 50.96 | C |
| ATOM | 4171 | CZ | PHE | B | 203 | −40.164 | 10.691 | 9.768 | 1.00 | 49.83 | C |
| ATOM | 4172 | CE1 | PHE | B | 203 | −40.134 | 11.448 | 10.918 | 1.00 | 45.03 | C |
| ATOM | 4173 | CD1 | PHE | B | 203 | −40.951 | 11.118 | 11.982 | 1.00 | 46.91 | C |
| ATOM | 4174 | C | PHE | B | 203 | −44.974 | 10.182 | 13.934 | 1.00 | 36.54 | C |
| ATOM | 4175 | O | PHE | B | 203 | −45.891 | 10.031 | 13.186 | 1.00 | 34.61 | O |
| ATOM | 4176 | N | PHE | B | 204 | −45.028 | 9.812 | 15.187 | 1.00 | 36.87 | N |
| ATOM | 4177 | CA | PHE | B | 204 | −46.212 | 9.215 | 15.788 | 1.00 | 34.99 | C |
| ATOM | 4178 | CB | PHE | B | 204 | −45.890 | 8.546 | 17.119 | 1.00 | 32.82 | C |
| ATOM | 4179 | CG | PHE | B | 204 | −45.296 | 7.178 | 16.959 | 1.00 | 31.79 | C |
| ATOM | 4180 | CD1 | PHE | B | 204 | −44.214 | 6.971 | 16.119 | 1.00 | 34.07 | C |
| ATOM | 4181 | CE1 | PHE | B | 204 | −43.665 | 5.714 | 15.967 | 1.00 | 33.60 | C |
| ATOM | 4182 | CZ | PHE | B | 204 | −44.190 | 4.648 | 16.648 | 1.00 | 31.79 | C |
| ATOM | 4183 | CE2 | PHE | B | 204 | −45.267 | 4.836 | 17.480 | 1.00 | 35.86 | C |
| ATOM | 4184 | CD2 | PHE | B | 204 | −45.817 | 6.097 | 17.634 | 1.00 | 34.92 | C |
| ATOM | 4185 | C | PHE | B | 204 | −47.415 | 10.161 | 15.866 | 1.00 | 39.88 | C |
| ATOM | 4186 | O | PHE | B | 204 | −48.494 | 9.791 | 15.505 | 1.00 | 44.57 | O |
| ATOM | 4187 | N | TRP | B | 205 | −47.227 | 11.415 | 16.256 | 1.00 | 37.47 | N |
| ATOM | 4188 | CA | TRP | B | 205 | −48.294 | 12.409 | 16.191 | 1.00 | 38.16 | C |
| ATOM | 4189 | CB | TRP | B | 205 | −47.922 | 13.688 | 16.937 | 1.00 | 35.92 | C |
| ATOM | 4190 | CG | TRP | B | 205 | −48.209 | 13.583 | 18.394 | 1.00 | 31.34 | C |
| ATOM | 4191 | CD1 | TRP | B | 205 | −47.299 | 13.566 | 19.405 | 1.00 | 33.32 | C |
| ATOM | 4192 | NE1 | TRP | B | 205 | −47.937 | 13.442 | 20.612 | 1.00 | 36.90 | N |
| ATOM | 4193 | CE2 | TRP | B | 205 | −49.286 | 13.364 | 20.397 | 1.00 | 36.02 | C |
| ATOM | 4194 | CD2 | TRP | B | 205 | −49.497 | 13.444 | 19.008 | 1.00 | 35.12 | C |
| ATOM | 4195 | CE3 | TRP | B | 205 | −50.807 | 13.386 | 18.522 | 1.00 | 35.18 | C |
| ATOM | 4196 | CZ3 | TRP | B | 205 | −51.846 | 13.254 | 19.432 | 1.00 | 40.24 | C |
| ATOM | 4197 | CH2 | TRP | B | 205 | −51.599 | 13.176 | 20.806 | 1.00 | 36.30 | C |
| ATOM | 4198 | CZ2 | TRP | B | 205 | −50.330 | 13.225 | 21.305 | 1.00 | 37.50 | C |
| ATOM | 4199 | C | TRP | B | 205 | −48.789 | 12.707 | 14.771 | 1.00 | 39.77 | C |
| ATOM | 4200 | O | TRP | B | 205 | −49.955 | 12.894 | 14.533 | 1.00 | 38.27 | O |
| ATOM | 4201 | N | MET | B | 206 | −47.889 | 12.695 | 13.813 | 1.00 | 40.73 | N |
| ATOM | 4202 | CA | MET | B | 206 | −48.247 | 12.784 | 12.408 | 1.00 | 39.70 | C |
| ATOM | 4203 | CB | MET | B | 206 | −46.995 | 12.789 | 11.529 | 1.00 | 35.20 | C |
| ATOM | 4204 | CG | MET | B | 206 | −46.235 | 14.099 | 11.516 | 1.00 | 40.91 | C |
| ATOM | 4205 | SD | MET | B | 206 | −47.151 | 15.441 | 10.747 | 1.00 | 46.26 | S |
| ATOM | 4206 | CE | MET | B | 206 | −45.927 | 16.755 | 10.796 | 1.00 | 40.35 | C |
| ATOM | 4207 | C | MET | B | 206 | −49.123 | 11.617 | 12.000 | 1.00 | 42.09 | C |
| ATOM | 4208 | O | MET | B | 206 | −50.041 | 11.756 | 11.232 | 1.00 | 45.81 | O |
| ATOM | 4209 | N | PHE | B | 207 | −48.821 | 10.451 | 12.537 | 1.00 | 40.69 | N |
| ATOM | 4210 | CA | PHE | B | 207 | −49.618 | 9.272 | 12.357 | 1.00 | 40.00 | C |
| ATOM | 4211 | CB | PHE | B | 207 | −48.845 | 8.106 | 12.958 | 1.00 | 40.97 | C |
| ATOM | 4212 | CG | PHE | B | 207 | −49.586 | 6.810 | 12.979 | 1.00 | 42.38 | C |
| ATOM | 4213 | CD1 | PHE | B | 207 | −49.943 | 6.175 | 11.805 | 1.00 | 44.49 | C |
| ATOM | 4214 | CE1 | PHE | B | 207 | −50.610 | 4.965 | 11.841 | 1.00 | 45.26 | C |
| ATOM | 4215 | CZ | PHE | B | 207 | −50.905 | 4.374 | 13.056 | 1.00 | 41.95 | C |
| ATOM | 4216 | CE2 | PHE | B | 207 | −50.538 | 4.993 | 14.226 | 1.00 | 39.53 | C |
| ATOM | 4217 | CD2 | PHE | B | 207 | −49.878 | 6.199 | 14.185 | 1.00 | 42.58 | C |
| ATOM | 4218 | C | PHE | B | 207 | −50.979 | 9.396 | 13.026 | 1.00 | 45.90 | C |
| ATOM | 4219 | O | PHE | B | 207 | −51.984 | 9.100 | 12.449 | 1.00 | 45.07 | O |
| ATOM | 4220 | N | GLY | B | 208 | −51.014 | 9.891 | 14.238 | 1.00 | 45.50 | N |
| ATOM | 4221 | CA | GLY | B | 208 | −52.247 | 10.188 | 14.906 | 1.00 | 40.93 | C |
| ATOM | 4222 | C | GLY | B | 208 | −53.139 | 11.108 | 14.159 | 1.00 | 42.39 | C |
| ATOM | 4223 | O | GLY | B | 208 | −54.329 | 10.925 | 14.104 | 1.00 | 51.79 | O |
| ATOM | 4224 | N | GLU | B | 209 | −52.561 | 12.066 | 13.488 | 1.00 | 42.60 | N |
| ATOM | 4225 | CA | GLU | B | 209 | −53.318 | 12.960 | 12.645 | 1.00 | 42.73 | C |
| ATOM | 4226 | CB | GLU | B | 209 | −52.387 | 13.983 | 11.998 | 1.00 | 38.10 | C |
| ATOM | 4227 | CG | GLU | B | 209 | −52.274 | 15.276 | 12.768 | 1.00 | 40.38 | C |
| ATOM | 4228 | CD | GLU | B | 209 | −53.600 | 15.994 | 12.855 | 1.00 | 48.58 | C |
| ATOM | 4229 | OE1 | GLU | B | 209 | −54.328 | 16.008 | 11.843 | 1.00 | 52.81 | O |
| ATOM | 4230 | OE2 | GLU | B | 209 | −53.921 | 16.535 | 13.933 | 1.00 | 57.64 | O |
| ATOM | 4231 | C | GLU | B | 209 | −54.081 | 12.191 | 11.547 | 1.00 | 47.26 | C |
| ATOM | 4232 | O | GLU | B | 209 | −55.281 | 12.316 | 11.402 | 1.00 | 47.89 | O |
| ATOM | 4233 | N | GLY | B | 210 | −53.374 | 11.315 | 10.848 | 1.00 | 48.83 | N |
| ATOM | 4234 | CA | GLY | B | 210 | −53.925 | 10.389 | 9.893 | 1.00 | 47.26 | C |
| ATOM | 4235 | C | GLY | B | 210 | −54.900 | 9.424 | 10.473 | 1.00 | 50.24 | C |
| ATOM | 4236 | O | GLY | B | 210 | −55.896 | 9.057 | 9.888 | 1.00 | 50.07 | O |
| ATOM | 4237 | N | CYS | B | 211 | −54.582 | 8.960 | 11.646 | 1.00 | 51.46 | N |
| ATOM | 4238 | CA | CYS | B | 211 | −55.359 | 7.948 | 12.310 | 1.00 | 56.12 | C |
| ATOM | 4239 | CB | CYS | B | 211 | −54.641 | 7.450 | 13.572 | 1.00 | 52.85 | C |
| ATOM | 4240 | SG | CYS | B | 211 | −54.061 | 5.719 | 13.504 | 1.00 | 56.38 | S |
| ATOM | 4241 | C | CYS | B | 211 | −56.734 | 8.482 | 12.652 | 1.00 | 54.35 | C |
| ATOM | 4242 | O | CYS | B | 211 | −57.748 | 7.830 | 12.440 | 1.00 | 63.25 | O |
| ATOM | 4243 | N | TYR | B | 212 | −56.741 | 9.735 | 13.095 | 1.00 | 47.39 | N |
| ATOM | 4244 | CA | TYR | B | 212 | −57.965 | 10.454 | 13.321 | 1.00 | 46.47 | C |
| ATOM | 4245 | CB | TYR | B | 212 | −57.689 | 11.826 | 13.932 | 1.00 | 50.39 | C |
| ATOM | 4246 | CG | TYR | B | 212 | −58.912 | 12.715 | 13.985 | 1.00 | 55.79 | C |
| ATOM | 4247 | CD1 | TYR | B | 212 | −59.938 | 12.468 | 14.890 | 1.00 | 49.37 | C |
| ATOM | 4248 | CE1 | TYR | B | 212 | −61.060 | 13.279 | 14.939 | 1.00 | 55.79 | C |
| ATOM | 4249 | CZ | TYR | B | 212 | −61.167 | 14.353 | 14.076 | 1.00 | 58.13 | C |
| ATOM | 4250 | OH | TYR | B | 212 | −62.280 | 15.165 | 14.116 | 1.00 | 65.11 | O |
| ATOM | 4251 | CE2 | TYR | B | 212 | −60.162 | 14.619 | 13.169 | 1.00 | 58.36 | C |
| ATOM | 4252 | CD2 | TYR | B | 212 | −59.044 | 13.802 | 13.126 | 1.00 | 56.08 | C |
| ATOM | 4253 | C | TYR | B | 212 | −58.761 | 10.628 | 12.063 | 1.00 | 47.91 | C |
| ATOM | 4254 | O | TYR | B | 212 | −59.947 | 10.455 | 12.060 | 1.00 | 54.12 | O |
| ATOM | 4255 | N | LEU | B | 213 | −58.097 | 11.124 | 11.042 | 1.00 | 50.43 | N |
| ATOM | 4256 | CA | LEU | B | 213 | −58.741 | 11.564 | 9.830 | 1.00 | 51.85 | C |
| ATOM | 4257 | CB | LEU | B | 213 | −57.721 | 12.244 | 8.916 | 1.00 | 53.15 | C |
| ATOM | 4258 | CG | LEU | B | 213 | −58.288 | 12.896 | 7.657 | 1.00 | 57.63 | C |
| ATOM | 4259 | CD1 | LEU | B | 213 | −59.317 | 13.961 | 8.018 | 1.00 | 63.49 | C |
| ATOM | 4260 | CD2 | LEU | B | 213 | −57.164 | 13.483 | 6.826 | 1.00 | 55.25 | C |
| ATOM | 4261 | C | LEU | B | 213 | −59.409 | 10.440 | 9.108 | 1.00 | 54.99 | C |
| ATOM | 4262 | O | LEU | B | 213 | −60.506 | 10.564 | 8.604 | 1.00 | 53.53 | O |
| ATOM | 4263 | N | HIS | B | 214 | −58.724 | 9.315 | 9.080 | 1.00 | 56.27 | N |
| ATOM | 4264 | CA | HIS | B | 214 | −59.232 | 8.127 | 8.450 | 1.00 | 56.14 | C |
| ATOM | 4265 | CB | HIS | B | 214 | −58.187 | 7.022 | 8.541 | 1.00 | 62.67 | C |
| ATOM | 4266 | CG | HIS | B | 214 | −58.531 | 5.809 | 7.740 | 1.00 | 71.46 | C |
| ATOM | 4267 | ND1 | HIS | B | 214 | −59.404 | 4.846 | 8.192 | 1.00 | 75.19 | N |
| ATOM | 4268 | CE1 | HIS | B | 214 | −59.523 | 3.902 | 7.274 | 1.00 | 85.02 | C |
| ATOM | 4269 | NE2 | HIS | B | 214 | −58.761 | 4.221 | 6.245 | 1.00 | 81.51 | N |
| ATOM | 4270 | CD2 | HIS | B | 214 | −58.130 | 5.414 | 6.510 | 1.00 | 73.42 | C |
| ATOM | 4271 | C | HIS | B | 214 | −60.535 | 7.658 | 9.090 | 1.00 | 51.27 | C |
| ATOM | 4272 | O | HIS | B | 214 | −61.542 | 7.509 | 8.418 | 1.00 | 59.15 | O |
| ATOM | 4273 | N | THR | B | 215 | −60.541 | 7.546 | 10.420 | 1.00 | 52.44 | N |
| ATOM | 4274 | CA | THR | B | 215 | −61.765 | 7.190 | 11.149 | 1.00 | 59.38 | C |
| ATOM | 4275 | CB | THR | B | 215 | −61.533 | 6.828 | 12.640 | 1.00 | 55.02 | C |
| ATOM | 4276 | OG1 | THR | B | 215 | −60.658 | 7.783 | 13.252 | 1.00 | 61.04 | O |
| ATOM | 4277 | CG2 | THR | B | 215 | −60.941 | 5.440 | 12.762 | 1.00 | 55.62 | C |
| ATOM | 4278 | C | THR | B | 215 | −62.850 | 8.270 | 11.040 | 1.00 | 46.95 | C |
| ATOM | 4279 | O | THR | B | 215 | −64.015 | 7.972 | 10.942 | 1.00 | 46.02 | O |
| ATOM | 4280 | N | ALA | B | 216 | −62.447 | 9.531 | 11.102 | 1.00 | 48.82 | N |
| ATOM | 4281 | CA | ALA | B | 216 | −63.344 | 10.663 | 11.004 | 1.00 | 48.34 | C |
| ATOM | 4282 | CB | ALA | B | 216 | −62.554 | 11.971 | 10.996 | 1.00 | 49.80 | C |
| ATOM | 4283 | C | ALA | B | 216 | −64.231 | 10.578 | 9.792 | 1.00 | 48.72 | C |
| ATOM | 4284 | O | ALA | B | 216 | −65.426 | 10.704 | 9.925 | 1.00 | 59.35 | O |
| ATOM | 4285 | N | ILE | B | 217 | −63.669 | 10.273 | 8.636 | 1.00 | 45.34 | N |
| ATOM | 4286 | CA | ILE | B | 217 | −64.491 | 10.012 | 7.462 | 1.00 | 43.66 | C |
| ATOM | 4287 | CB | ILE | B | 217 | −63.643 | 9.850 | 6.196 | 1.00 | 49.81 | C |
| ATOM | 4288 | CG1 | ILE | B | 217 | −62.969 | 11.170 | 5.838 | 1.00 | 52.20 | C |
| ATOM | 4289 | CD1 | ILE | B | 217 | −62.204 | 11.114 | 4.536 | 1.00 | 49.54 | C |
| ATOM | 4290 | CG2 | ILE | B | 217 | −64.503 | 9.375 | 5.035 | 1.00 | 55.05 | C |
| ATOM | 4291 | C | ILE | B | 217 | −65.394 | 8.749 | 7.615 | 1.00 | 44.68 | C |
| ATOM | 4292 | O | ILE | B | 217 | −66.574 | 8.768 | 7.325 | 1.00 | 52.96 | O |
| ATOM | 4293 | N | VAL | B | 218 | −64.811 | 7.614 | 7.967 | 1.00 | 45.29 | N |
| ATOM | 4294 | CA | VAL | B | 218 | −65.569 | 6.361 | 8.056 | 1.00 | 45.67 | C |
| ATOM | 4295 | CB | VAL | B | 218 | −64.617 | 5.168 | 8.377 | 1.00 | 50.28 | C |
| ATOM | 4296 | CG1 | VAL | B | 218 | −65.360 | 3.836 | 8.393 | 1.00 | 57.85 | C |
| ATOM | 4297 | CG2 | VAL | B | 218 | −63.487 | 5.116 | 7.362 | 1.00 | 50.23 | C |
| ATOM | 4298 | C | VAL | B | 218 | −66.777 | 6.382 | 9.011 | 1.00 | 45.97 | C |
| ATOM | 4299 | O | VAL | B | 218 | −67.737 | 5.676 | 8.780 | 1.00 | 48.70 | O |
| ATOM | 4300 | N | LEU | B | 219 | −66.641 | 7.036 | 10.166 | 1.00 | 47.05 | N |
| ATOM | 4301 | CA | LEU | B | 219 | −67.669 | 6.996 | 11.216 | 1.00 | 45.70 | C |
| ATOM | 4302 | CB | LEU | B | 219 | −67.126 | 6.314 | 12.476 | 1.00 | 47.22 | C |
| ATOM | 4303 | CG | LEU | B | 219 | −66.475 | 4.934 | 12.320 | 1.00 | 51.45 | C |
| ATOM | 4304 | CD1 | LEU | B | 219 | −65.955 | 4.432 | 13.658 | 1.00 | 46.93 | C |
| ATOM | 4305 | CD2 | LEU | B | 219 | −67.433 | 3.921 | 11.707 | 1.00 | 46.52 | C |
| ATOM | 4306 | C | LEU | B | 219 | −68.187 | 8.386 | 11.571 | 1.00 | 46.31 | C |
| ATOM | 4307 | O | LEU | B | 219 | −67.528 | 9.366 | 11.326 | 1.00 | 51.15 | O |
| ATOM | 4308 | N | THR | B | 220 | −69.404 | 8.470 | 12.129 | 1.00 | 46.87 | N |
| ATOM | 4309 | CA | THR | B | 220 | −70.000 | 9.756 | 12.494 | 1.00 | 42.92 | C |
| ATOM | 4310 | CB | THR | B | 220 | −71.512 | 9.812 | 12.153 | 1.00 | 41.15 | C |
| ATOM | 4311 | OG1 | THR | B | 220 | −72.202 | 8.719 | 12.776 | 1.00 | 41.24 | O |
| ATOM | 4312 | CG2 | THR | B | 220 | −71.720 | 9.740 | 10.652 | 1.00 | 47.33 | C |
| ATOM | 4313 | C | THR | B | 220 | −69.792 | 10.026 | 13.972 | 1.00 | 43.20 | C |
| ATOM | 4314 | O | THR | B | 220 | −69.442 | 9.140 | 14.711 | 1.00 | 42.80 | O |
| ATOM | 4315 | N | ASN | B | 1002 | −70.017 | 11.249 | 14.409 | 1.00 | 36.30 | N |
| ATOM | 4316 | CA | ASN | B | 1002 | −69.833 | 11.585 | 15.799 | 1.00 | 38.20 | C |
| ATOM | 4317 | CB | ASN | B | 1002 | −69.945 | 13.095 | 15.985 | 1.00 | 47.92 | C |
| ATOM | 4318 | CG | ASN | B | 1002 | −68.738 | 13.828 | 15.448 | 1.00 | 51.43 | C |
| ATOM | 4319 | OD1 | ASN | B | 1002 | −67.595 | 13.464 | 15.747 | 1.00 | 52.59 | O |
| ATOM | 4320 | ND2 | ASN | B | 1002 | −68.977 | 14.859 | 14.645 | 1.00 | 53.43 | N |
| ATOM | 4321 | C | ASN | B | 1002 | −70.825 | 10.879 | 16.694 | 1.00 | 44.10 | C |
| ATOM | 4322 | O | ASN | B | 1002 | −70.486 | 10.382 | 17.744 | 1.00 | 39.75 | O |
| ATOM | 4323 | N | ILE | B | 1003 | −72.068 | 10.816 | 16.230 | 1.00 | 47.92 | N |
| ATOM | 4324 | CA | ILE | B | 1003 | −73.155 | 10.188 | 16.960 | 1.00 | 43.06 | C |
| ATOM | 4325 | CB | ILE | B | 1003 | −74.523 | 10.509 | 16.333 | 1.00 | 42.05 | C |
| ATOM | 4326 | CG1 | ILE | B | 1003 | −75.651 | 10.049 | 17.260 | 1.00 | 34.10 | C |
| ATOM | 4327 | CD1 | ILE | B | 1003 | −76.973 | 10.734 | 16.989 | 1.00 | 28.96 | C |
| ATOM | 4328 | CG2 | ILE | B | 1003 | −74.638 | 9.901 | 14.940 | 1.00 | 47.76 | C |
| ATOM | 4329 | C | ILE | B | 1003 | −72.962 | 8.686 | 17.151 | 1.00 | 44.05 | C |
| ATOM | 4330 | O | ILE | B | 1003 | −73.255 | 8.162 | 18.210 | 1.00 | 46.97 | O |
| ATOM | 4331 | N | PHE | B | 1004 | −72.340 | 8.032 | 16.163 | 1.00 | 38.52 | N |
| ATOM | 4332 | CA | PHE | B | 1004 | −71.921 | 6.640 | 16.280 | 1.00 | 41.00 | C |
| ATOM | 4333 | CB | PHE | B | 1004 | −71.369 | 6.137 | 14.949 | 1.00 | 44.18 | C |
| ATOM | 4334 | CG | PHE | B | 1004 | −70.966 | 4.692 | 14.961 | 1.00 | 49.16 | C |
| ATOM | 4335 | CD1 | PHE | B | 1004 | −71.910 | 3.694 | 14.783 | 1.00 | 48.95 | C |
| ATOM | 4336 | CE1 | PHE | B | 1004 | −71.538 | 2.358 | 14.781 | 1.00 | 49.86 | C |
| ATOM | 4337 | CZ | PHE | B | 1004 | −70.213 | 2.008 | 14.954 | 1.00 | 49.06 | C |
| ATOM | 4338 | CE2 | PHE | B | 1004 | −69.260 | 2.994 | 15.128 | 1.00 | 53.83 | C |
| ATOM | 4339 | CD2 | PHE | B | 1004 | −69.638 | 4.329 | 15.128 | 1.00 | 51.25 | C |
| ATOM | 4340 | C | PHE | B | 1004 | −70.895 | 6.458 | 17.400 | 1.00 | 45.51 | C |
| ATOM | 4341 | O | PHE | B | 1004 | −70.993 | 5.551 | 18.198 | 1.00 | 47.67 | O |
| ATOM | 4342 | N | GLU | B | 1005 | −69.884 | 7.322 | 17.431 | 1.00 | 41.94 | N |
| ATOM | 4343 | CA | GLU | B | 1005 | −68.852 | 7.282 | 18.459 | 1.00 | 45.27 | C |
| ATOM | 4344 | CB | GLU | B | 1005 | −67.687 | 8.212 | 18.105 | 1.00 | 43.12 | C |
| ATOM | 4345 | CG | GLU | B | 1005 | −66.707 | 7.605 | 17.107 | 1.00 | 53.05 | C |
| ATOM | 4346 | CD | GLU | B | 1005 | −66.168 | 6.250 | 17.558 | 1.00 | 56.61 | C |
| ATOM | 4347 | OE1 | GLU | B | 1005 | −65.757 | 6.124 | 18.733 | 1.00 | 53.32 | O |
| ATOM | 4348 | OE2 | GLU | B | 1005 | −66.163 | 5.306 | 16.737 | 1.00 | 50.48 | O |
| ATOM | 4349 | C | GLU | B | 1005 | −69.370 | 7.586 | 19.854 | 1.00 | 42.52 | C |
| ATOM | 4350 | O | GLU | B | 1005 | −68.952 | 6.981 | 20.828 | 1.00 | 39.06 | O |
| ATOM | 4351 | N | MET | B | 1006 | −70.345 | 8.474 | 19.930 | 1.00 | 44.54 | N |
| ATOM | 4352 | CA | MET | B | 1006 | −71.005 | 8.790 | 21.185 | 1.00 | 44.85 | C |
| ATOM | 4353 | CB | MET | B | 1006 | −72.089 | 9.840 | 20.923 | 1.00 | 38.31 | C |
| ATOM | 4354 | CG | MET | B | 1006 | −72.768 | 10.395 | 22.158 | 1.00 | 36.45 | C |
| ATOM | 4355 | SD | MET | B | 1006 | −74.081 | 11.553 | 21.720 | 1.00 | 43.09 | S |
| ATOM | 4356 | CE | MET | B | 1006 | −74.564 | 12.171 | 23.327 | 1.00 | 39.19 | C |
| ATOM | 4357 | C | MET | B | 1006 | −71.625 | 7.562 | 21.856 | 1.00 | 44.19 | C |
| ATOM | 4358 | O | MET | B | 1006 | −71.401 | 7.281 | 23.025 | 1.00 | 44.15 | O |
| ATOM | 4359 | N | LEU | B | 1007 | −72.439 | 6.846 | 21.092 | 1.00 | 38.53 | N |
| ATOM | 4360 | CA | LEU | B | 1007 | −73.117 | 5.674 | 21.575 | 1.00 | 37.01 | C |
| ATOM | 4361 | CB | LEU | B | 1007 | −74.374 | 5.363 | 20.764 | 1.00 | 40.80 | C |
| ATOM | 4362 | CG | LEU | B | 1007 | −75.563 | 6.128 | 21.362 | 1.00 | 37.38 | C |
| ATOM | 4363 | CD1 | LEU | B | 1007 | −75.517 | 7.608 | 21.004 | 1.00 | 36.57 | C |
| ATOM | 4364 | CD2 | LEU | B | 1007 | −76.882 | 5.517 | 20.952 | 1.00 | 45.59 | C |
| ATOM | 4365 | C | LEU | B | 1007 | −72.221 | 4.494 | 21.755 | 1.00 | 34.93 | C |
| ATOM | 4366 | O | LEU | B | 1007 | −72.332 | 3.814 | 22.726 | 1.00 | 40.33 | O |
| ATOM | 4367 | N | ARG | B | 1008 | −71.285 | 4.289 | 20.850 | 1.00 | 39.22 | N |
| ATOM | 4368 | CA | ARG | B | 1008 | −70.184 | 3.318 | 21.041 | 1.00 | 42.84 | C |
| ATOM | 4369 | CB | ARG | B | 1008 | −69.126 | 3.454 | 19.949 | 1.00 | 44.95 | C |
| ATOM | 4370 | CG | ARG | B | 1008 | −67.996 | 2.452 | 20.089 | 1.00 | 52.68 | C |
| ATOM | 4371 | CD | ARG | B | 1008 | −66.917 | 2.660 | 19.040 | 1.00 | 61.54 | C |
| ATOM | 4372 | NE | ARG | B | 1008 | −65.875 | 1.641 | 19.151 | 1.00 | 69.74 | N |
| ATOM | 4373 | CZ | ARG | B | 1008 | −64.873 | 1.493 | 18.290 | 1.00 | 65.60 | C |
| ATOM | 4374 | NH1 | ARG | B | 1008 | −64.771 | 2.304 | 17.245 | 1.00 | 60.92 | N |
| ATOM | 4375 | NH2 | ARG | B | 1008 | −63.975 | 0.532 | 18.470 | 1.00 | 65.54 | N |
| ATOM | 4376 | C | ARG | B | 1008 | −69.528 | 3.440 | 22.418 | 1.00 | 37.45 | C |
| ATOM | 4377 | O | ARG | B | 1008 | −69.353 | 2.479 | 23.124 | 1.00 | 31.22 | O |
| ATOM | 4378 | N | ILE | B | 1009 | −69.181 | 4.655 | 22.796 | 1.00 | 35.78 | N |
| ATOM | 4379 | CA | ILE | B | 1009 | −68.616 | 4.896 | 24.102 | 1.00 | 37.42 | C |
| ATOM | 4380 | CB | ILE | B | 1009 | −68.139 | 6.349 | 24.235 | 1.00 | 38.84 | C |
| ATOM | 4381 | CG1 | ILE | B | 1009 | −66.972 | 6.591 | 23.278 | 1.00 | 36.32 | C |
| ATOM | 4382 | CD1 | ILE | B | 1009 | −66.565 | 8.039 | 23.152 | 1.00 | 33.25 | C |
| ATOM | 4383 | CG2 | ILE | B | 1009 | −67.729 | 6.654 | 25.669 | 1.00 | 33.27 | C |
| ATOM | 4384 | C | ILE | B | 1009 | −69.597 | 4.560 | 25.232 | 1.00 | 37.99 | C |
| ATOM | 4385 | O | ILE | B | 1009 | −69.251 | 3.889 | 26.191 | 1.00 | 40.28 | O |
| ATOM | 4386 | N | ASP | B | 1010 | −70.818 | 5.090 | 25.143 | 1.00 | 39.52 | N |
| ATOM | 4387 | CA | ASP | B | 1010 | −71.816 | 4.918 | 26.189 | 1.00 | 37.85 | C |
| ATOM | 4388 | CB | ASP | B | 1010 | −72.978 | 5.887 | 25.985 | 1.00 | 38.18 | C |
| ATOM | 4389 | CG | ASP | B | 1010 | −72.627 | 7.301 | 26.391 | 1.00 | 40.05 | C |
| ATOM | 4390 | OD1 | ASP | B | 1010 | −71.602 | 7.481 | 27.080 | 1.00 | 40.58 | O |
| ATOM | 4391 | OD2 | ASP | B | 1010 | −73.374 | 8.233 | 26.035 | 1.00 | 40.10 | O |
| ATOM | 4392 | C | ASP | B | 1010 | −72.350 | 3.489 | 26.324 | 1.00 | 38.67 | C |
| ATOM | 4393 | O | ASP | B | 1010 | −72.277 | 2.942 | 27.405 | 1.00 | 48.40 | O |
| ATOM | 4394 | N | GLU | B | 1011 | −72.868 | 2.886 | 25.242 | 1.00 | 38.40 | N |
| ATOM | 4395 | CA | GLU | B | 1011 | −73.372 | 1.506 | 25.278 | 1.00 | 38.12 | C |
| ATOM | 4396 | CB | GLU | B | 1011 | −74.560 | 1.376 | 24.331 | 1.00 | 42.31 | C |
| ATOM | 4397 | CG | GLU | B | 1011 | −75.680 | 2.330 | 24.672 | 1.00 | 48.86 | C |
| ATOM | 4398 | CD | GLU | B | 1011 | −76.846 | 2.227 | 23.721 | 1.00 | 54.26 | C |
| ATOM | 4399 | OE1 | GLU | B | 1011 | −76.773 | 1.422 | 22.767 | 1.00 | 58.72 | O |
| ATOM | 4400 | OE2 | GLU | B | 1011 | −77.838 | 2.954 | 23.933 | 1.00 | 52.88 | O |
| ATOM | 4401 | C | GLU | B | 1011 | −72.353 | 0.372 | 24.981 | 1.00 | 41.72 | C |
| ATOM | 4402 | O | GLU | B | 1011 | −72.493 | −0.740 | 25.452 | 1.00 | 53.84 | O |
| ATOM | 4403 | N | GLY | B | 1012 | −71.380 | 0.621 | 24.137 | 1.00 | 42.34 | N |
| ATOM | 4404 | CA | GLY | B | 1012 | −70.437 | −0.400 | 23.702 | 1.00 | 42.49 | C |
| ATOM | 4405 | C | GLY | B | 1012 | −70.827 | −1.031 | 22.406 | 1.00 | 51.21 | C |
| ATOM | 4406 | O | GLY | B | 1012 | −71.919 | −0.764 | 21.988 | 1.00 | 62.58 | O |
| ATOM | 4407 | N | LEU | B | 1013 | −69.934 | −1.758 | 21.721 | 1.00 | 51.22 | N |
| ATOM | 4408 | CA | LEU | B | 1013 | −70.167 | −2.218 | 20.349 | 1.00 | 49.01 | C |
| ATOM | 4409 | CB | LEU | B | 1013 | −69.410 | −1.320 | 19.365 | 1.00 | 49.44 | C |
| ATOM | 4410 | CG | LEU | B | 1013 | −69.240 | −1.782 | 17.916 | 1.00 | 50.55 | C |
| ATOM | 4411 | CD1 | LEU | B | 1013 | −70.554 | −1.703 | 17.155 | 1.00 | 53.66 | C |
| ATOM | 4412 | CD2 | LEU | B | 1013 | −68.155 | −0.968 | 17.215 | 1.00 | 48.41 | C |
| ATOM | 4413 | C | LEU | B | 1013 | −69.739 | −3.687 | 20.170 | 1.00 | 53.88 | C |
| ATOM | 4414 | O | LEU | B | 1013 | −68.615 | −4.070 | 20.461 | 1.00 | 65.16 | O |
| ATOM | 4415 | N | ARG | B | 1014 | −70.704 | −4.519 | 19.774 | 1.00 | 49.69 | N |
| ATOM | 4416 | CA | ARG | B | 1014 | −70.534 | −5.954 | 19.646 | 1.00 | 50.22 | C |
| ATOM | 4417 | CB | ARG | B | 1014 | −71.651 | −6.665 | 20.404 | 1.00 | 55.38 | C |
| ATOM | 4418 | CG | ARG | B | 1014 | −71.813 | −6.186 | 21.836 | 1.00 | 63.65 | C |
| ATOM | 4419 | CD | ARG | B | 1014 | −70.697 | −6.703 | 22.714 | 1.00 | 64.13 | C |
| ATOM | 4420 | NE | ARG | B | 1014 | −70.703 | −8.160 | 22.764 | 1.00 | 62.71 | N |
| ATOM | 4421 | CZ | ARG | B | 1014 | −71.416 | −8.876 | 23.626 | 1.00 | 58.59 | C |
| ATOM | 4422 | NH1 | ARG | B | 1014 | −71.354 | −10.200 | 23.589 | 1.00 | 55.76 | N |
| ATOM | 4423 | NH2 | ARG | B | 1014 | −72.188 | −8.271 | 24.523 | 1.00 | 58.89 | N |
| ATOM | 4424 | C | ARG | B | 1014 | −70.514 | −6.431 | 18.185 | 1.00 | 58.86 | C |
| ATOM | 4425 | O | ARG | B | 1014 | −71.511 | −6.357 | 17.515 | 1.00 | 58.62 | O |
| ATOM | 4426 | N | LEU | B | 1015 | −69.374 | −6.915 | 17.699 | 1.00 | 63.30 | N |
| ATOM | 4427 | CA | LEU | B | 1015 | −69.201 | −7.406 | 16.319 | 1.00 | 56.62 | C |
| ATOM | 4428 | CB | LEU | B | 1015 | −67.716 | −7.503 | 15.981 | 1.00 | 69.60 | C |
| ATOM | 4429 | CG | LEU | B | 1015 | −67.078 | −6.110 | 15.992 | 1.00 | 70.01 | C |
| ATOM | 4430 | CD1 | LEU | B | 1015 | −65.558 | −6.185 | 15.905 | 1.00 | 66.81 | C |
| ATOM | 4431 | CD2 | LEU | B | 1015 | −67.662 | −5.260 | 14.863 | 1.00 | 56.90 | C |
| ATOM | 4432 | C | LEU | B | 1015 | −69.958 | −8.707 | 15.950 | 1.00 | 54.88 | C |
| ATOM | 4433 | O | LEU | B | 1015 | −70.442 | −8.842 | 14.834 | 1.00 | 51.63 | O |
| ATOM | 4434 | N | LYS | B | 1016 | −70.050 | −9.651 | 16.905 | 1.00 | 54.26 | N |
| ATOM | 4435 | CA | LYS | B | 1016 | −70.790 | −10.926 | 16.721 | 1.00 | 57.94 | C |
| ATOM | 4436 | CB | LYS | B | 1016 | −70.010 | −12.101 | 17.315 | 1.00 | 59.07 | C |
| ATOM | 4437 | CG | LYS | B | 1016 | −68.676 | −12.424 | 16.670 | 1.00 | 70.77 | C |
| ATOM | 4438 | CD | LYS | B | 1016 | −68.120 | −13.713 | 17.263 | 1.00 | 73.04 | C |
| ATOM | 4439 | CE | LYS | B | 1016 | −66.743 | −14.048 | 16.720 | 1.00 | 73.28 | C |
| ATOM | 4440 | NZ | LYS | B | 1016 | −65.702 | −13.115 | 17.225 | 1.00 | 73.89 | N |
| ATOM | 4441 | C | LYS | B | 1016 | −72.178 | −10.879 | 17.419 | 1.00 | 58.08 | C |
| ATOM | 4442 | O | LYS | B | 1016 | −72.341 | −10.196 | 18.416 | 1.00 | 56.10 | O |
| ATOM | 4443 | N | ILE | B | 1017 | −73.151 | −11.675 | 16.925 | 1.00 | 57.57 | N |
| ATOM | 4444 | CA | ILE | B | 1017 | −74.457 | −11.851 | 17.603 | 1.00 | 54.67 | C |
| ATOM | 4445 | CB | ILE | B | 1017 | −75.331 | −12.898 | 16.868 | 1.00 | 57.11 | C |
| ATOM | 4446 | CG1 | ILE | B | 1017 | −75.613 | −12.454 | 15.432 | 1.00 | 63.14 | C |
| ATOM | 4447 | CD1 | ILE | B | 1017 | −76.637 | −13.308 | 14.721 | 1.00 | 71.34 | C |
| ATOM | 4448 | CG2 | ILE | B | 1017 | −76.637 | −13.131 | 17.606 | 1.00 | 59.29 | C |
| ATOM | 4449 | C | ILE | B | 1017 | −74.329 | −12.250 | 19.083 | 1.00 | 51.10 | C |
| ATOM | 4450 | O | ILE | B | 1017 | −73.533 | −13.099 | 19.429 | 1.00 | 57.26 | O |
| ATOM | 4451 | N | TYR | B | 1018 | −75.156 | −11.631 | 19.942 | 1.00 | 50.85 | N |
| ATOM | 4452 | CA | TYR | B | 1018 | −75.162 | −11.906 | 21.368 | 1.00 | 50.69 | C |
| ATOM | 4453 | CB | TYR | B | 1018 | −74.198 | −10.984 | 22.114 | 1.00 | 50.45 | C |
| ATOM | 4454 | CG | TYR | B | 1018 | −74.686 | −9.574 | 22.312 | 1.00 | 48.50 | C |
| ATOM | 4455 | CD2 | TYR | B | 1018 | −75.154 | −9.153 | 23.547 | 1.00 | 49.56 | C |
| ATOM | 4456 | CE2 | TYR | B | 1018 | −75.585 | −7.860 | 23.743 | 1.00 | 53.22 | C |
| ATOM | 4457 | CZ | TYR | B | 1018 | −75.542 | −6.967 | 22.699 | 1.00 | 53.81 | C |
| ATOM | 4458 | OH | TYR | B | 1018 | −75.969 | −5.674 | 22.891 | 1.00 | 49.68 | O |
| ATOM | 4459 | CE1 | TYR | B | 1018 | −75.075 | −7.359 | 21.463 | 1.00 | 59.14 | C |
| ATOM | 4460 | CD1 | TYR | B | 1018 | −74.645 | −8.654 | 21.277 | 1.00 | 56.27 | C |
| ATOM | 4461 | C | TYR | B | 1018 | −76.564 | −11.813 | 21.930 | 1.00 | 57.31 | C |
| ATOM | 4462 | O | TYR | B | 1018 | −77.437 | −11.224 | 21.341 | 1.00 | 62.09 | O |
| ATOM | 4463 | N | LYS | B | 1019 | −76.761 | −12.409 | 23.086 | 1.00 | 59.08 | N |
| ATOM | 4464 | CA | LYS | B | 1019 | −78.035 | −12.388 | 23.767 | 1.00 | 59.95 | C |
| ATOM | 4465 | CB | LYS | B | 1019 | −78.352 | −13.754 | 24.376 | 1.00 | 64.43 | C |
| ATOM | 4466 | CG | LYS | B | 1019 | −78.679 | −14.858 | 23.389 | 1.00 | 57.60 | C |
| ATOM | 4467 | CD | LYS | B | 1019 | −79.007 | −16.130 | 24.152 | 1.00 | 60.95 | C |
| ATOM | 4468 | CE | LYS | B | 1019 | −79.165 | −17.325 | 23.235 | 1.00 | 76.54 | C |
| ATOM | 4469 | NZ | LYS | B | 1019 | −79.325 | −18.590 | 24.009 | 1.00 | 97.48 | N |
| ATOM | 4470 | C | LYS | B | 1019 | −78.029 | −11.342 | 24.878 | 1.00 | 57.78 | C |
| ATOM | 4471 | O | LYS | B | 1019 | −77.142 | −11.334 | 25.717 | 1.00 | 61.29 | O |
| ATOM | 4472 | N | ASP | B | 1020 | −79.016 | −10.445 | 24.877 | 1.00 | 50.60 | N |
| ATOM | 4473 | CA | ASP | B | 1020 | −79.117 | −9.458 | 25.933 | 1.00 | 56.18 | C |
| ATOM | 4474 | CB | ASP | B | 1020 | −80.075 | −8.319 | 25.549 | 1.00 | 62.70 | C |
| ATOM | 4475 | CG | ASP | B | 1020 | −81.410 | −8.816 | 25.013 | 1.00 | 59.05 | C |
| ATOM | 4476 | OD1 | ASP | B | 1020 | −81.852 | −9.915 | 25.404 | 1.00 | 61.09 | O |
| ATOM | 4477 | OD2 | ASP | B | 1020 | −82.024 | −8.092 | 24.198 | 1.00 | 56.24 | O |
| ATOM | 4478 | C | ASP | B | 1020 | −79.543 | −10.111 | 27.236 | 1.00 | 60.43 | C |
| ATOM | 4479 | O | ASP | B | 1020 | −79.530 | −11.319 | 27.335 | 1.00 | 59.78 | O |
| ATOM | 4480 | N | THR | B | 1021 | −79.895 | −9.322 | 28.248 | 1.00 | 60.66 | N |
| ATOM | 4481 | CA | THR | B | 1021 | −80.248 | −9.889 | 29.540 | 1.00 | 61.01 | C |
| ATOM | 4482 | CB | THR | B | 1021 | −80.260 | −8.824 | 30.657 | 1.00 | 63.61 | C |
| ATOM | 4483 | OG1 | THR | B | 1021 | −81.172 | −7.774 | 30.312 | 1.00 | 57.88 | O |
| ATOM | 4484 | CG2 | THR | B | 1021 | −78.867 | −8.240 | 30.861 | 1.00 | 63.63 | C |
| ATOM | 4485 | C | THR | B | 1021 | −81.602 | −10.607 | 29.508 | 1.00 | 63.70 | C |
| ATOM | 4486 | O | THR | B | 1021 | −81.842 | −11.507 | 30.303 | 1.00 | 62.60 | O |
| ATOM | 4487 | N | GLU | B | 1022 | −82.471 | −10.191 | 28.573 | 1.00 | 61.84 | N |
| ATOM | 4488 | CA | GLU | B | 1022 | −83.788 | −10.780 | 28.395 | 1.00 | 54.06 | C |
| ATOM | 4489 | CB | GLU | B | 1022 | −84.812 | −9.734 | 27.928 | 1.00 | 57.17 | C |
| ATOM | 4490 | CG | GLU | B | 1022 | −84.934 | −8.550 | 28.876 | 1.00 | 59.72 | C |
| ATOM | 4491 | CD | GLU | B | 1022 | −86.095 | −7.618 | 28.555 | 1.00 | 61.88 | C |
| ATOM | 4492 | OE1 | GLU | B | 1022 | −86.743 | −7.806 | 27.505 | 1.00 | 72.43 | O |
| ATOM | 4493 | OE2 | GLU | B | 1022 | −86.360 | −6.693 | 29.357 | 1.00 | 57.76 | O |
| ATOM | 4494 | C | GLU | B | 1022 | −83.765 | −11.988 | 27.446 | 1.00 | 61.96 | C |
| ATOM | 4495 | O | GLU | B | 1022 | −84.752 | −12.694 | 27.303 | 1.00 | 65.35 | O |
| ATOM | 4496 | N | GLY | B | 1023 | −82.602 | −12.248 | 26.836 | 1.00 | 68.22 | N |
| ATOM | 4497 | CA | GLY | B | 1023 | −82.370 | −13.422 | 26.020 | 1.00 | 63.22 | C |
| ATOM | 4498 | C | GLY | B | 1023 | −82.576 | −13.232 | 24.557 | 1.00 | 64.87 | C |
| ATOM | 4499 | O | GLY | B | 1023 | −82.387 | −14.158 | 23.795 | 1.00 | 71.80 | O |
| ATOM | 4500 | N | TYR | B | 1024 | −82.947 | −12.035 | 24.133 | 1.00 | 66.96 | N |
| ATOM | 4501 | CA | TYR | B | 1024 | −83.150 | −11.755 | 22.721 | 1.00 | 67.86 | C |
| ATOM | 4502 | CB | TYR | B | 1024 | −84.032 | −10.522 | 22.569 | 1.00 | 62.66 | C |
| ATOM | 4503 | CG | TYR | B | 1024 | −85.399 | −10.649 | 23.197 | 1.00 | 64.85 | C |
| ATOM | 4504 | CD2 | TYR | B | 1024 | −85.898 | −9.648 | 24.020 | 1.00 | 64.70 | C |
| ATOM | 4505 | CE2 | TYR | B | 1024 | −87.147 | −9.752 | 24.597 | 1.00 | 65.39 | C |
| ATOM | 4506 | CZ | TYR | B | 1024 | −87.918 | −10.869 | 24.357 | 1.00 | 69.64 | C |
| ATOM | 4507 | OH | TYR | B | 1024 | −89.165 | −10.965 | 24.931 | 1.00 | 68.45 | O |
| ATOM | 4508 | CE1 | TYR | B | 1024 | −87.447 | −11.881 | 23.548 | 1.00 | 74.53 | C |
| ATOM | 4509 | CD1 | TYR | B | 1024 | −86.191 | −11.768 | 22.972 | 1.00 | 69.90 | C |
| ATOM | 4510 | C | TYR | B | 1024 | −81.830 | −11.557 | 21.996 | 1.00 | 64.31 | C |
| ATOM | 4511 | O | TYR | B | 1024 | −80.868 | −11.094 | 22.572 | 1.00 | 58.93 | O |
| ATOM | 4512 | N | TYR | B | 1025 | −81.820 | −11.895 | 20.711 | 1.00 | 61.20 | N |
| ATOM | 4513 | CA | TYR | B | 1025 | −80.649 | −11.751 | 19.879 | 1.00 | 61.89 | C |
| ATOM | 4514 | CB | TYR | B | 1025 | −80.688 | −12.730 | 18.706 | 1.00 | 67.94 | C |
| ATOM | 4515 | CG | TYR | B | 1025 | −80.513 | −14.172 | 19.125 | 1.00 | 79.51 | C |
| ATOM | 4516 | CD2 | TYR | B | 1025 | −81.562 | −15.082 | 19.031 | 1.00 | 85.92 | C |
| ATOM | 4517 | CE2 | TYR | B | 1025 | −81.399 | −16.403 | 19.410 | 1.00 | 89.12 | C |
| ATOM | 4518 | CZ | TYR | B | 1025 | −80.177 | −16.825 | 19.889 | 1.00 | 87.75 | C |
| ATOM | 4519 | OH | TYR | B | 1025 | −80.003 | −18.137 | 20.268 | 1.00 | 89.40 | O |
| ATOM | 4520 | CE1 | TYR | B | 1025 | −79.124 | −15.940 | 19.989 | 1.00 | 84.92 | C |
| ATOM | 4521 | CD1 | TYR | B | 1025 | −79.295 | −14.626 | 19.609 | 1.00 | 77.69 | C |
| ATOM | 4522 | C | TYR | B | 1025 | −80.410 | −10.310 | 19.404 | 1.00 | 61.85 | C |
| ATOM | 4523 | O | TYR | B | 1025 | −81.279 | −9.670 | 18.839 | 1.00 | 60.27 | O |
| ATOM | 4524 | N | THR | B | 1026 | −79.209 | −9.820 | 19.719 | 1.00 | 58.08 | N |
| ATOM | 4525 | CA | THR | B | 1026 | −78.792 | −8.434 | 19.560 | 1.00 | 55.46 | C |
| ATOM | 4526 | CB | THR | B | 1026 | −78.614 | −7.812 | 20.963 | 1.00 | 50.23 | C |
| ATOM | 4527 | OG1 | THR | B | 1026 | −79.775 | −8.088 | 21.758 | 1.00 | 57.44 | O |
| ATOM | 4528 | CG2 | THR | B | 1026 | −78.388 | −6.310 | 20.904 | 1.00 | 50.74 | C |
| ATOM | 4529 | C | THR | B | 1026 | −77.445 | −8.407 | 18.818 | 1.00 | 56.95 | C |
| ATOM | 4530 | O | THR | B | 1026 | −76.700 | −9.353 | 18.883 | 1.00 | 58.55 | O |
| ATOM | 4531 | N | ILE | B | 1027 | −77.112 | −7.315 | 18.147 | 1.00 | 51.00 | N |
| ATOM | 4532 | CA | ILE | B | 1027 | −75.790 | −7.164 | 17.578 | 1.00 | 48.19 | C |
| ATOM | 4533 | CB | ILE | B | 1027 | −75.645 | −7.902 | 16.224 | 1.00 | 57.15 | C |
| ATOM | 4534 | CG1 | ILE | B | 1027 | −74.170 | −8.191 | 15.923 | 1.00 | 51.69 | C |
| ATOM | 4535 | CD1 | ILE | B | 1027 | −73.939 | −9.047 | 14.698 | 1.00 | 54.09 | C |
| ATOM | 4536 | CG2 | ILE | B | 1027 | −76.306 | −7.116 | 15.102 | 1.00 | 60.41 | C |
| ATOM | 4537 | C | ILE | B | 1027 | −75.493 | −5.688 | 17.430 | 1.00 | 54.55 | C |
| ATOM | 4538 | O | ILE | B | 1027 | −76.395 | −4.895 | 17.378 | 1.00 | 60.38 | O |
| ATOM | 4539 | N | GLY | B | 1028 | −74.233 | −5.324 | 17.307 | 1.00 | 57.20 | N |
| ATOM | 4540 | CA | GLY | B | 1028 | −73.835 | −3.954 | 17.111 | 1.00 | 51.17 | C |
| ATOM | 4541 | C | GLY | B | 1028 | −73.974 | −3.083 | 18.295 | 1.00 | 52.02 | C |
| ATOM | 4542 | O | GLY | B | 1028 | −73.686 | −3.539 | 19.389 | 1.00 | 57.12 | O |
| ATOM | 4543 | N | ILE | B | 1029 | −74.417 | −1.834 | 18.105 | 1.00 | 51.27 | N |
| ATOM | 4544 | CA | ILE | B | 1029 | −74.642 | −0.948 | 19.225 | 1.00 | 51.54 | C |
| ATOM | 4545 | CB | ILE | B | 1029 | −74.413 | 0.530 | 18.864 | 1.00 | 42.34 | C |
| ATOM | 4546 | CG1 | ILE | B | 1029 | −73.053 | 0.708 | 18.195 | 1.00 | 43.57 | C |
| ATOM | 4547 | CD1 | ILE | B | 1029 | −72.636 | 2.157 | 18.050 | 1.00 | 45.30 | C |
| ATOM | 4548 | CG2 | ILE | B | 1029 | −74.493 | 1.404 | 20.110 | 1.00 | 42.70 | C |
| ATOM | 4549 | C | ILE | B | 1029 | −76.052 | −1.150 | 19.740 | 1.00 | 59.73 | C |
| ATOM | 4550 | O | ILE | B | 1029 | −76.927 | −0.306 | 19.614 | 1.00 | 62.73 | O |
| ATOM | 4551 | N | GLY | B | 1030 | −76.256 | −2.337 | 20.298 | 1.00 | 57.60 | N |
| ATOM | 4552 | CA | GLY | B | 1030 | −77.469 | −2.737 | 20.906 | 1.00 | 53.29 | C |
| ATOM | 4553 | C | GLY | B | 1030 | −78.630 | −2.807 | 19.991 | 1.00 | 51.98 | C |
| ATOM | 4554 | O | GLY | B | 1030 | −79.685 | −2.358 | 20.395 | 1.00 | 61.58 | O |
| ATOM | 4555 | N | HIS | B | 1031 | −78.534 | −3.364 | 18.800 | 1.00 | 49.53 | N |
| ATOM | 4556 | CA | HIS | B | 1031 | −79.706 | −3.373 | 17.985 | 1.00 | 50.26 | C |
| ATOM | 4557 | CB | HIS | B | 1031 | −79.281 | −2.962 | 16.570 | 1.00 | 46.03 | C |
| ATOM | 4558 | CG | HIS | B | 1031 | −80.262 | −3.319 | 15.497 | 1.00 | 56.26 | C |
| ATOM | 4559 | ND1 | HIS | B | 1031 | −81.342 | −2.526 | 15.181 | 1.00 | 59.58 | N |
| ATOM | 4560 | CE1 | HIS | B | 1031 | −82.020 | −3.086 | 14.193 | 1.00 | 58.39 | C |
| ATOM | 4561 | NE2 | HIS | B | 1031 | −81.413 | −4.206 | 13.854 | 1.00 | 64.89 | N |
| ATOM | 4562 | CD2 | HIS | B | 1031 | −80.304 | −4.375 | 14.651 | 1.00 | 58.81 | C |
| ATOM | 4563 | C | HIS | B | 1031 | −80.209 | −4.765 | 17.991 | 1.00 | 55.06 | C |
| ATOM | 4564 | O | HIS | B | 1031 | −79.463 | −5.676 | 18.236 | 1.00 | 58.20 | O |
| ATOM | 4565 | N | LEU | B | 1032 | −81.509 | −4.900 | 17.855 | 1.00 | 56.82 | N |
| ATOM | 4566 | CA | LEU | B | 1032 | −82.132 | −6.170 | 18.076 | 1.00 | 58.05 | C |
| ATOM | 4567 | CB | LEU | B | 1032 | −83.228 | −5.946 | 19.112 | 1.00 | 56.71 | C |
| ATOM | 4568 | CG | LEU | B | 1032 | −84.108 | −7.108 | 19.536 | 1.00 | 59.56 | C |
| ATOM | 4569 | CD1 | LEU | B | 1032 | −83.310 | −7.970 | 20.468 | 1.00 | 61.93 | C |
| ATOM | 4570 | CD2 | LEU | B | 1032 | −85.367 | −6.590 | 20.213 | 1.00 | 53.83 | C |
| ATOM | 4571 | C | LEU | B | 1032 | −82.778 | −6.633 | 16.810 | 1.00 | 63.38 | C |
| ATOM | 4572 | O | LEU | B | 1032 | −83.239 | −5.848 | 15.994 | 1.00 | 62.58 | O |
| ATOM | 4573 | N | LEU | B | 1033 | −82.643 | −7.919 | 16.569 | 1.00 | 66.08 | N |
| ATOM | 4574 | CA | LEU | B | 1033 | −83.036 | −8.442 | 15.301 | 1.00 | 79.51 | C |
| ATOM | 4575 | CB | LEU | B | 1033 | −82.043 | −9.521 | 14.859 | 1.00 | 82.08 | C |
| ATOM | 4576 | CG | LEU | B | 1033 | −80.578 | −9.069 | 14.822 | 1.00 | 69.69 | C |
| ATOM | 4577 | CD1 | LEU | B | 1033 | −79.636 | −10.251 | 14.657 | 1.00 | 77.64 | C |
| ATOM | 4578 | CD2 | LEU | B | 1033 | −80.355 | −8.044 | 13.718 | 1.00 | 57.56 | C |
| ATOM | 4579 | C | LEU | B | 1033 | −84.443 | −9.010 | 15.386 | 1.00 | 86.69 | C |
| ATOM | 4580 | O | LEU | B | 1033 | −85.360 | −8.554 | 14.704 | 1.00 | 85.56 | O |
| ATOM | 4581 | N | THR | B | 1034 | −84.618 | −9.956 | 16.301 | 1.00 | 82.04 | N |
| ATOM | 4582 | CA | THR | B | 1034 | −85.910 | −10.510 | 16.521 | 1.00 | 82.56 | C |
| ATOM | 4583 | CB | THR | B | 1034 | −86.253 | −11.592 | 15.480 | 1.00 | 89.95 | C |
| ATOM | 4584 | OG1 | THR | B | 1034 | −87.595 | −12.054 | 15.688 | 1.00 | 90.60 | O |
| ATOM | 4585 | CG2 | THR | B | 1034 | −85.287 | −12.763 | 15.591 | 1.00 | 87.48 | C |
| ATOM | 4586 | C | THR | B | 1034 | −85.954 | −11.126 | 17.912 | 1.00 | 82.79 | C |
| ATOM | 4587 | O | THR | B | 1034 | −84.928 | −11.340 | 18.550 | 1.00 | 74.87 | O |
| ATOM | 4588 | N | LYS | B | 1035 | −87.156 | −11.550 | 18.297 | 1.00 | 92.05 | N |
| ATOM | 4589 | CA | LYS | B | 1035 | −87.358 | −12.285 | 19.525 | 1.00 | 87.81 | C |
| ATOM | 4590 | CB | LYS | B | 1035 | −88.508 | −11.665 | 20.318 | 1.00 | 71.72 | C |
| ATOM | 4591 | CG | LYS | B | 1035 | −88.317 | −10.186 | 20.616 | 1.00 | 65.14 | C |
| ATOM | 4592 | CD | LYS | B | 1035 | −89.444 | −9.632 | 21.471 | 1.00 | 64.34 | C |
| ATOM | 4593 | CE | LYS | B | 1035 | −89.165 | −8.190 | 21.871 | 1.00 | 59.02 | C |
| ATOM | 4594 | NZ | LYS | B | 1035 | −90.246 | −7.622 | 22.722 | 1.00 | 79.04 | N |
| ATOM | 4595 | C | LYS | B | 1035 | −87.687 | −13.733 | 19.156 | 1.00 | 91.67 | C |
| ATOM | 4596 | O | LYS | B | 1035 | −88.710 | −14.015 | 18.544 | 1.00 | 87.31 | O |
| ATOM | 4597 | N | SER | B | 1036 | −86.732 | −14.620 | 19.400 | 1.00 | 93.42 | N |
| ATOM | 4598 | CA | SER | B | 1036 | −86.860 | −15.997 | 18.951 | 1.00 | 92.29 | C |
| ATOM | 4599 | CB | SER | B | 1036 | −86.701 | −16.099 | 17.431 | 1.00 | 93.39 | C |
| ATOM | 4600 | OG | SER | B | 1036 | −86.693 | −17.450 | 17.006 | 1.00 | 92.22 | O |
| ATOM | 4601 | C | SER | B | 1036 | −85.783 | −16.822 | 19.632 | 1.00 | 96.03 | C |
| ATOM | 4602 | O | SER | B | 1036 | −84.614 | −16.459 | 19.560 | 1.00 | 105.69 | O |
| ATOM | 4603 | N | PRO | B | 1037 | −86.075 | −17.905 | 20.341 | 1.00 | 91.82 | N |
| ATOM | 4604 | CA | PRO | B | 1037 | −85.046 | −18.690 | 21.000 | 1.00 | 99.67 | C |
| ATOM | 4605 | CB | PRO | B | 1037 | −85.848 | −19.668 | 21.870 | 1.00 | 107.68 | C |
| ATOM | 4606 | CG | PRO | B | 1037 | −87.192 | −19.041 | 22.027 | 1.00 | 105.21 | C |
| ATOM | 4607 | CD | PRO | B | 1037 | −87.421 | −18.351 | 20.715 | 1.00 | 97.40 | C |
| ATOM | 4608 | C | PRO | B | 1037 | −84.184 | −19.469 | 19.979 | 1.00 | 102.52 | C |
| ATOM | 4609 | O | PRO | B | 1037 | −83.645 | −20.496 | 20.378 | 1.00 | 111.38 | O |
| ATOM | 4610 | N | SER | B | 1038 | −83.973 | −19.020 | 18.733 | 1.00 | 95.38 | N |
| ATOM | 4611 | CA | SER | B | 1038 | −83.090 | −19.769 | 17.867 | 1.00 | 98.26 | C |
| ATOM | 4612 | CB | SER | B | 1038 | −83.887 | −20.605 | 16.867 | 1.00 | 104.68 | C |
| ATOM | 4613 | OG | SER | B | 1038 | −83.030 | −21.402 | 16.067 | 1.00 | 110.27 | O |
| ATOM | 4614 | C | SER | B | 1038 | −82.122 | −18.830 | 17.152 | 1.00 | 103.61 | C |
| ATOM | 4615 | O | SER | B | 1038 | −82.517 | −17.913 | 16.435 | 1.00 | 101.03 | O |
| ATOM | 4616 | N | LEU | B | 1039 | −80.823 | −19.161 | 17.317 | 1.00 | 107.53 | N |
| ATOM | 4617 | CA | LEU | B | 1039 | −79.701 | −18.471 | 16.667 | 1.00 | 103.85 | C |
| ATOM | 4618 | CB | LEU | B | 1039 | −78.360 | −18.996 | 17.186 | 1.00 | 99.26 | C |
| ATOM | 4619 | CG | LEU | B | 1039 | −77.141 | −18.200 | 16.719 | 1.00 | 95.17 | C |
| ATOM | 4620 | CD1 | LEU | B | 1039 | −77.360 | −16.709 | 16.948 | 1.00 | 87.51 | C |
| ATOM | 4621 | CD2 | LEU | B | 1039 | −75.878 | −18.675 | 17.423 | 1.00 | 89.68 | C |
| ATOM | 4622 | C | LEU | B | 1039 | −79.753 | −18.546 | 15.150 | 1.00 | 112.95 | C |
| ATOM | 4623 | O | LEU | B | 1039 | −79.463 | −17.583 | 14.438 | 1.00 | 116.19 | O |
| ATOM | 4624 | N | SER | B | 1040 | −80.086 | −19.755 | 14.673 | 1.00 | 115.12 | N |
| ATOM | 4625 | CA | SER | B | 1040 | −80.131 | −20.057 | 13.248 | 1.00 | 112.47 | C |
| ATOM | 4626 | CB | SER | B | 1040 | −80.440 | −21.538 | 13.018 | 1.00 | 117.24 | C |
| ATOM | 4627 | OG | SER | B | 1040 | −79.400 | −22.361 | 13.518 | 1.00 | 122.89 | O |
| ATOM | 4628 | C | SER | B | 1040 | −81.176 | −19.167 | 12.534 | 1.00 | 108.90 | C |
| ATOM | 4629 | O | SER | B | 1040 | −80.900 | −18.548 | 11.490 | 1.00 | 117.67 | O |
| ATOM | 4630 | N | VAL | B | 1041 | −82.322 | −19.002 | 13.201 | 1.00 | 100.69 | N |
| ATOM | 4631 | CA | VAL | B | 1041 | −83.340 | −18.097 | 12.724 | 1.00 | 108.18 | C |
| ATOM | 4632 | CB | VAL | B | 1041 | −84.485 | −18.190 | 13.656 | 1.00 | 111.42 | C |
| ATOM | 4633 | CG1 | VAL | B | 1041 | −85.617 | −17.248 | 13.209 | 1.00 | 105.64 | C |
| ATOM | 4634 | CG2 | VAL | B | 1041 | −84.859 | −19.651 | 13.720 | 1.00 | 111.31 | C |
| ATOM | 4635 | C | VAL | B | 1041 | −82.835 | −16.660 | 12.774 | 1.00 | 111.68 | C |
| ATOM | 4636 | O | VAL | B | 1041 | −83.165 | −15.868 | 11.902 | 1.00 | 108.83 | O |
| ATOM | 4637 | N | ALA | B | 1042 | −82.107 | −16.318 | 13.856 | 1.00 | 115.69 | N |
| ATOM | 4638 | CA | ALA | B | 1042 | −81.619 | −14.938 | 14.078 | 1.00 | 111.75 | C |
| ATOM | 4639 | CB | ALA | B | 1042 | −81.035 | −14.800 | 15.479 | 1.00 | 101.08 | C |
| ATOM | 4640 | C | ALA | B | 1042 | −80.610 | −14.457 | 13.022 | 1.00 | 107.70 | C |
| ATOM | 4641 | O | ALA | B | 1042 | −80.695 | −13.323 | 12.559 | 1.00 | 102.07 | O |
| ATOM | 4642 | N | LYS | B | 1043 | −79.704 | −15.360 | 12.603 | 1.00 | 109.11 | N |
| ATOM | 4643 | CA | LYS | B | 1043 | −78.717 | −15.074 | 11.544 | 1.00 | 106.17 | C |
| ATOM | 4644 | CB | LYS | B | 1043 | −77.786 | −16.269 | 11.323 | 1.00 | 101.03 | C |
| ATOM | 4645 | CG | LYS | B | 1043 | −76.820 | −16.520 | 12.467 | 1.00 | 99.72 | C |
| ATOM | 4646 | CD | LYS | B | 1043 | −76.125 | −17.863 | 12.325 | 1.00 | 90.86 | C |
| ATOM | 4647 | CE | LYS | B | 1043 | −75.238 | −18.143 | 13.526 | 1.00 | 80.27 | C |
| ATOM | 4648 | NZ | LYS | B | 1043 | −74.777 | −19.557 | 13.547 | 1.00 | 82.25 | N |
| ATOM | 4649 | C | LYS | B | 1043 | −79.388 | −14.652 | 10.203 | 1.00 | 111.36 | C |
| ATOM | 4650 | O | LYS | B | 1043 | −79.014 | −13.655 | 9.581 | 1.00 | 109.67 | O |
| ATOM | 4651 | N | SER | B | 1044 | −80.434 | −15.404 | 9.800 | 1.00 | 111.36 | N |
| ATOM | 4652 | CA | SER | B | 1044 | −81.199 | −15.094 | 8.578 | 1.00 | 107.74 | C |
| ATOM | 4653 | CB | SER | B | 1044 | −82.282 | −16.146 | 8.342 | 1.00 | 112.32 | C |
| ATOM | 4654 | OG | SER | B | 1044 | −81.714 | −17.441 | 8.241 | 1.00 | 124.82 | O |
| ATOM | 4655 | C | SER | B | 1044 | −81.832 | −13.673 | 8.615 | 1.00 | 107.69 | C |
| ATOM | 4656 | O | SER | B | 1044 | −81.816 | −12.925 | 7.631 | 1.00 | 107.36 | O |
| ATOM | 4657 | N | GLU | B | 1045 | −82.365 | −13.318 | 9.793 | 1.00 | 106.83 | N |
| ATOM | 4658 | CA | GLU | B | 1045 | −82.903 | −11.986 | 10.053 | 1.00 | 107.56 | C |
| ATOM | 4659 | CB | GLU | B | 1045 | −83.485 | −11.912 | 11.467 | 1.00 | 103.90 | C |
| ATOM | 4660 | CG | GLU | B | 1045 | −84.743 | −12.735 | 11.648 | 1.00 | 110.39 | C |
| ATOM | 4661 | CD | GLU | B | 1045 | −85.847 | −12.308 | 10.701 | 1.00 | 122.24 | C |
| ATOM | 4662 | OE1 | GLU | B | 1045 | −86.303 | −11.149 | 10.803 | 1.00 | 122.18 | O |
| ATOM | 4663 | OE2 | GLU | B | 1045 | −86.251 | −13.127 | 9.848 | 1.00 | 123.81 | O |
| ATOM | 4664 | C | GLU | B | 1045 | −81.867 | −10.865 | 9.852 | 1.00 | 103.45 | C |
| ATOM | 4665 | O | GLU | B | 1045 | −82.170 | −9.816 | 9.277 | 1.00 | 94.23 | O |
| ATOM | 4666 | N | LEU | B | 1046 | −80.656 | −11.088 | 10.408 | 1.00 | 98.11 | N |
| ATOM | 4667 | CA | LEU | B | 1046 | −79.557 | −10.113 | 10.334 | 1.00 | 93.56 | C |
| ATOM | 4668 | CB | LEU | B | 1046 | −78.348 | −10.618 | 11.126 | 1.00 | 91.83 | C |
| ATOM | 4669 | CG | LEU | B | 1046 | −77.062 | −9.790 | 11.089 | 1.00 | 76.48 | C |
| ATOM | 4670 | CD1 | LEU | B | 1046 | −77.315 | −8.370 | 11.564 | 1.00 | 73.55 | C |
| ATOM | 4671 | CD2 | LEU | B | 1046 | −75.988 | −10.455 | 11.936 | 1.00 | 75.24 | C |
| ATOM | 4672 | C | LEU | B | 1046 | −79.165 | −9.831 | 8.901 | 1.00 | 89.89 | C |
| ATOM | 4673 | O | LEU | B | 1046 | −79.104 | −8.685 | 8.474 | 1.00 | 81.72 | O |
| ATOM | 4674 | N | ASP | B | 1047 | −79.015 | −10.930 | 8.158 | 1.00 | 95.07 | N |
| ATOM | 4675 | CA | ASP | B | 1047 | −78.743 | −10.911 | 6.726 | 1.00 | 91.74 | C |
| ATOM | 4676 | CB | ASP | B | 1047 | −78.788 | −12.327 | 6.139 | 1.00 | 89.08 | C |
| ATOM | 4677 | CG | ASP | B | 1047 | −77.878 | −13.290 | 6.865 | 1.00 | 96.03 | C |
| ATOM | 4678 | OD1 | ASP | B | 1047 | −76.950 | −12.816 | 7.554 | 1.00 | 92.20 | O |
| ATOM | 4679 | OD2 | ASP | B | 1047 | −78.090 | −14.518 | 6.752 | 1.00 | 103.99 | O |
| ATOM | 4680 | C | ASP | B | 1047 | −79.714 | −10.020 | 5.935 | 1.00 | 87.11 | C |
| ATOM | 4681 | O | ASP | B | 1047 | −79.310 | −9.189 | 5.134 | 1.00 | 90.68 | O |
| ATOM | 4682 | N | LYS | B | 1048 | −81.007 | −10.180 | 6.196 | 1.00 | 84.44 | N |
| ATOM | 4683 | CA | LYS | B | 1048 | −82.018 | −9.370 | 5.530 | 1.00 | 85.69 | C |
| ATOM | 4684 | CB | LYS | B | 1048 | −83.420 | −9.735 | 5.962 | 1.00 | 98.80 | C |
| ATOM | 4685 | CG | LYS | B | 1048 | −84.435 | −8.928 | 5.224 | 1.00 | 95.39 | C |
| ATOM | 4686 | CD | LYS | B | 1048 | −85.788 | −9.352 | 5.623 | 1.00 | 97.77 | C |
| ATOM | 4687 | CE | LYS | B | 1048 | −86.787 | −8.547 | 4.886 | 1.00 | 111.37 | C |
| ATOM | 4688 | NZ | LYS | B | 1048 | −88.022 | −9.299 | 5.009 | 1.00 | 123.99 | N |
| ATOM | 4689 | C | LYS | B | 1048 | −81.779 | −7.858 | 5.742 | 1.00 | 77.18 | C |
| ATOM | 4690 | O | LYS | B | 1048 | −81.765 | −7.073 | 4.803 | 1.00 | 71.61 | O |
| ATOM | 4691 | N | ALA | B | 1049 | −81.636 | −7.464 | 7.010 | 1.00 | 81.65 | N |
| ATOM | 4692 | CA | ALA | B | 1049 | −81.403 | −6.056 | 7.362 | 1.00 | 86.94 | C |
| ATOM | 4693 | CB | ALA | B | 1049 | −81.573 | −5.852 | 8.860 | 1.00 | 85.82 | C |
| ATOM | 4694 | C | ALA | B | 1049 | −80.035 | −5.522 | 6.900 | 1.00 | 85.57 | C |
| ATOM | 4695 | O | ALA | B | 1049 | −79.891 | −4.359 | 6.535 | 1.00 | 88.20 | O |
| ATOM | 4696 | N | ILE | B | 1050 | −79.015 | −6.375 | 6.998 | 1.00 | 82.70 | N |
| ATOM | 4697 | CA | ILE | B | 1050 | −77.675 | −6.038 | 6.531 | 1.00 | 74.71 | C |
| ATOM | 4698 | CB | ILE | B | 1050 | −76.643 | −7.082 | 7.054 | 1.00 | 69.36 | C |
| ATOM | 4699 | CG1 | ILE | B | 1050 | −76.278 | −6.792 | 8.507 | 1.00 | 67.82 | C |
| ATOM | 4700 | CD1 | ILE | B | 1050 | −75.581 | −5.470 | 8.700 | 1.00 | 70.24 | C |
| ATOM | 4701 | CG2 | ILE | B | 1050 | −75.376 | −7.096 | 6.223 | 1.00 | 68.42 | C |
| ATOM | 4702 | C | ILE | B | 1050 | −77.583 | −5.936 | 5.015 | 1.00 | 76.93 | C |
| ATOM | 4703 | O | ILE | B | 1050 | −76.847 | −5.108 | 4.499 | 1.00 | 85.85 | O |
| ATOM | 4704 | N | GLY | B | 1051 | −78.196 | −6.906 | 4.322 | 1.00 | 83.21 | N |
| ATOM | 4705 | CA | GLY | B | 1051 | −78.012 | −7.071 | 2.897 | 1.00 | 75.94 | C |
| ATOM | 4706 | C | GLY | B | 1051 | −77.278 | −8.348 | 2.576 | 1.00 | 73.89 | C |
| ATOM | 4707 | O | GLY | B | 1051 | −77.786 | −9.179 | 1.854 | 1.00 | 85.93 | O |
| ATOM | 4708 | N | ARG | B | 1052 | −76.062 | −8.487 | 3.035 | 1.00 | 79.69 | N |
| ATOM | 4709 | CA | ARG | B | 1052 | −75.277 | −9.667 | 2.752 | 1.00 | 86.56 | C |
| ATOM | 4710 | CB | ARG | B | 1052 | −73.777 | −9.339 | 2.808 | 1.00 | 87.40 | C |
| ATOM | 4711 | CG | ARG | B | 1052 | −73.315 | −8.325 | 1.772 | 1.00 | 92.59 | C |
| ATOM | 4712 | CD | ARG | B | 1052 | −71.889 | −7.872 | 2.012 | 1.00 | 96.92 | C |
| ATOM | 4713 | NE | ARG | B | 1052 | −71.412 | −6.998 | 0.942 | 1.00 | 111.85 | N |
| ATOM | 4714 | CZ | ARG | B | 1052 | −71.569 | −5.677 | 0.928 | 1.00 | 112.38 | C |
| ATOM | 4715 | NH1 | ARG | B | 1052 | −72.197 | −5.067 | 1.927 | 1.00 | 105.91 | N |
| ATOM | 4716 | NH2 | ARG | B | 1052 | −71.097 | −4.964 | −0.087 | 1.00 | 114.71 | N |
| ATOM | 4717 | C | ARG | B | 1052 | −75.586 | −10.778 | 3.770 | 1.00 | 89.41 | C |
| ATOM | 4718 | O | ARG | B | 1052 | −75.795 | −10.495 | 4.942 | 1.00 | 94.85 | O |
| ATOM | 4719 | N | ASN | B | 1053 | −75.436 | −12.056 | 3.371 | 1.00 | 94.60 | N |
| ATOM | 4720 | CA | ASN | B | 1053 | −75.323 | −13.117 | 4.386 | 1.00 | 100.02 | C |
| ATOM | 4721 | CB | ASN | B | 1053 | −75.188 | −14.495 | 3.727 | 1.00 | 103.17 | C |
| ATOM | 4722 | CG | ASN | B | 1053 | −75.139 | −15.632 | 4.741 | 1.00 | 98.97 | C |
| ATOM | 4723 | OD1 | ASN | B | 1053 | −75.581 | −15.487 | 5.881 | 1.00 | 98.97 | O |
| ATOM | 4724 | ND2 | ASN | B | 1053 | −74.601 | −16.774 | 4.322 | 1.00 | 93.68 | N |
| ATOM | 4725 | C | ASN | B | 1053 | −74.106 | −12.826 | 5.306 | 1.00 | 89.70 | C |
| ATOM | 4726 | O | ASN | B | 1053 | −73.061 | −12.415 | 4.824 | 1.00 | 85.52 | O |
| ATOM | 4727 | N | SER | B | 1054 | −74.276 | −12.957 | 6.617 | 1.00 | 90.61 | N |
| ATOM | 4728 | CA | SER | B | 1054 | −73.255 | −12.498 | 7.529 | 1.00 | 89.16 | C |
| ATOM | 4729 | CB | SER | B | 1054 | −73.789 | −11.349 | 8.385 | 1.00 | 90.33 | C |
| ATOM | 4730 | OG | SER | B | 1054 | −74.887 | −11.777 | 9.170 | 1.00 | 100.91 | O |
| ATOM | 4731 | C | SER | B | 1054 | −72.708 | −13.612 | 8.432 | 1.00 | 93.90 | C |
| ATOM | 4732 | O | SER | B | 1054 | −71.660 | −13.456 | 9.057 | 1.00 | 92.64 | O |
| ATOM | 4733 | N | ASN | B | 1055 | −73.447 | −14.724 | 8.512 | 1.00 | 97.23 | N |
| ATOM | 4734 | CA | ASN | B | 1055 | −73.119 | −15.831 | 9.414 | 1.00 | 102.67 | C |
| ATOM | 4735 | CB | ASN | B | 1055 | −71.983 | −16.676 | 8.836 | 1.00 | 104.52 | C |
| ATOM | 4736 | CG | ASN | B | 1055 | −72.428 | −17.492 | 7.642 | 1.00 | 112.55 | C |
| ATOM | 4737 | OD1 | ASN | B | 1055 | −73.599 | −17.875 | 7.540 | 1.00 | 112.98 | O |
| ATOM | 4738 | ND2 | ASN | B | 1055 | −71.502 | −17.768 | 6.733 | 1.00 | 111.75 | N |
| ATOM | 4739 | C | ASN | B | 1055 | −72.784 | −15.384 | 10.838 | 1.00 | 98.26 | C |
| ATOM | 4740 | O | ASN | B | 1055 | −71.918 | −15.954 | 11.501 | 1.00 | 87.47 | O |
| ATOM | 4741 | N | GLY | B | 1056 | −73.539 | −14.387 | 11.304 | 1.00 | 96.31 | N |
| ATOM | 4742 | CA | GLY | B | 1056 | −73.511 | −13.939 | 12.679 | 1.00 | 90.71 | C |
| ATOM | 4743 | C | GLY | B | 1056 | −72.445 | −12.910 | 13.028 | 1.00 | 88.29 | C |
| ATOM | 4744 | O | GLY | B | 1056 | −72.265 | −12.611 | 14.207 | 1.00 | 87.21 | O |
| ATOM | 4745 | N | VAL | B | 1057 | −71.748 | −12.372 | 12.023 | 1.00 | 83.55 | N |
| ATOM | 4746 | CA | VAL | B | 1057 | −70.697 | −11.426 | 12.253 | 1.00 | 66.75 | C |
| ATOM | 4747 | CB | VAL | B | 1057 | −69.302 | −12.077 | 12.090 | 1.00 | 70.00 | C |
| ATOM | 4748 | CG1 | VAL | B | 1057 | −68.201 | −11.075 | 12.420 | 1.00 | 78.81 | C |
| ATOM | 4749 | CG2 | VAL | B | 1057 | −69.172 | −13.318 | 12.962 | 1.00 | 71.83 | C |
| ATOM | 4750 | C | VAL | B | 1057 | −70.825 | −10.323 | 11.237 | 1.00 | 70.38 | C |
| ATOM | 4751 | O | VAL | B | 1057 | −70.949 | −10.603 | 10.062 | 1.00 | 76.88 | O |
| ATOM | 4752 | N | ILE | B | 1058 | −70.817 | −9.072 | 11.697 | 1.00 | 64.15 | N |
| ATOM | 4753 | CA | ILE | B | 1058 | −70.939 | −7.926 | 10.811 | 1.00 | 63.22 | C |
| ATOM | 4754 | CB | ILE | B | 1058 | −72.209 | −7.103 | 11.099 | 1.00 | 58.83 | C |
| ATOM | 4755 | CG1 | ILE | B | 1058 | −72.223 | −6.621 | 12.549 | 1.00 | 54.10 | C |
| ATOM | 4756 | CD1 | ILE | B | 1058 | −73.397 | −5.741 | 12.883 | 1.00 | 54.67 | C |
| ATOM | 4757 | CG2 | ILE | B | 1058 | −73.442 | −7.927 | 10.799 | 1.00 | 64.19 | C |
| ATOM | 4758 | C | ILE | B | 1058 | −69.712 | −7.053 | 10.966 | 1.00 | 62.83 | C |
| ATOM | 4759 | O | ILE | B | 1058 | −68.994 | −7.152 | 11.949 | 1.00 | 63.00 | O |
| ATOM | 4760 | N | THR | B | 1059 | −69.473 | −6.207 | 9.961 | 1.00 | 59.97 | N |
| ATOM | 4761 | CA | THR | B | 1059 | −68.336 | −5.311 | 9.966 | 1.00 | 62.06 | C |
| ATOM | 4762 | CB | THR | B | 1059 | −67.854 | −5.004 | 8.537 | 1.00 | 63.44 | C |
| ATOM | 4763 | OG1 | THR | B | 1059 | −68.916 | −4.402 | 7.788 | 1.00 | 61.76 | O |
| ATOM | 4764 | CG2 | THR | B | 1059 | −67.419 | −6.280 | 7.841 | 1.00 | 67.10 | C |
| ATOM | 4765 | C | THR | B | 1059 | −68.694 | −4.004 | 10.666 | 1.00 | 57.64 | C |
| ATOM | 4766 | O | THR | B | 1059 | −69.847 | −3.681 | 10.818 | 1.00 | 55.96 | O |
| ATOM | 4767 | N | LYS | B | 1060 | −67.692 | −3.315 | 11.192 | 1.00 | 62.98 | N |
| ATOM | 4768 | CA | LYS | B | 1060 | −67.883 | −2.080 | 11.920 | 1.00 | 51.95 | C |
| ATOM | 4769 | CB | LYS | B | 1060 | −66.532 | −1.525 | 12.383 | 1.00 | 51.42 | C |
| ATOM | 4770 | CG | LYS | B | 1060 | −66.579 | −0.099 | 12.908 | 1.00 | 51.88 | C |
| ATOM | 4771 | CD | LYS | B | 1060 | −65.460 | 0.170 | 13.908 | 1.00 | 60.55 | C |
| ATOM | 4772 | CE | LYS | B | 1060 | −64.092 | −0.224 | 13.364 | 1.00 | 63.15 | C |
| ATOM | 4773 | NZ | LYS | B | 1060 | −63.574 | 0.748 | 12.363 | 1.00 | 79.62 | N |
| ATOM | 4774 | C | LYS | B | 1060 | −68.636 | −1.050 | 11.090 | 1.00 | 57.47 | C |
| ATOM | 4775 | O | LYS | B | 1060 | −69.522 | −0.386 | 11.571 | 1.00 | 62.57 | O |
| ATOM | 4776 | N | ASP | B | 1061 | −68.337 | −0.981 | 9.809 | 1.00 | 65.40 | N |
| ATOM | 4777 | CA | ASP | B | 1061 | −69.070 | −0.119 | 8.874 | 1.00 | 68.74 | C |
| ATOM | 4778 | CB | ASP | B | 1061 | −68.407 | −0.145 | 7.496 | 1.00 | 82.77 | C |
| ATOM | 4779 | CG | ASP | B | 1061 | −68.890 | 0.974 | 6.595 | 1.00 | 92.25 | C |
| ATOM | 4780 | OD1 | ASP | B | 1061 | −70.053 | 0.915 | 6.138 | 1.00 | 90.95 | O |
| ATOM | 4781 | OD2 | ASP | B | 1061 | −68.102 | 1.910 | 6.337 | 1.00 | 93.82 | O |
| ATOM | 4782 | C | ASP | B | 1061 | −70.567 | −0.504 | 8.751 | 1.00 | 64.64 | C |
| ATOM | 4783 | O | ASP | B | 1061 | −71.449 | 0.338 | 8.766 | 1.00 | 64.17 | O |
| ATOM | 4784 | N | GLU | B | 1062 | −70.835 | −1.805 | 8.680 | 1.00 | 66.23 | N |
| ATOM | 4785 | CA | GLU | B | 1062 | −72.195 | −2.323 | 8.681 | 1.00 | 62.30 | C |
| ATOM | 4786 | CB | GLU | B | 1062 | −72.198 | −3.834 | 8.414 | 1.00 | 57.45 | C |
| ATOM | 4787 | CG | GLU | B | 1062 | −71.940 | −4.240 | 6.968 | 1.00 | 63.66 | C |
| ATOM | 4788 | CD | GLU | B | 1062 | −71.800 | −5.747 | 6.804 | 1.00 | 62.27 | C |
| ATOM | 4789 | OE1 | GLU | B | 1062 | −71.690 | −6.451 | 7.830 | 1.00 | 58.32 | O |
| ATOM | 4790 | OE2 | GLU | B | 1062 | −71.799 | −6.228 | 5.650 | 1.00 | 67.25 | O |
| ATOM | 4791 | C | GLU | B | 1062 | −72.916 | −2.057 | 10.000 | 1.00 | 55.70 | C |
| ATOM | 4792 | O | GLU | B | 1062 | −74.098 | −1.834 | 10.009 | 1.00 | 55.49 | O |
| ATOM | 4793 | N | ALA | B | 1063 | −72.187 | −2.074 | 11.107 | 1.00 | 54.65 | N |
| ATOM | 4794 | CA | ALA | B | 1063 | −72.716 | −1.703 | 12.411 | 1.00 | 50.25 | C |
| ATOM | 4795 | CB | ALA | B | 1063 | −71.635 | −1.823 | 13.477 | 1.00 | 51.30 | C |
| ATOM | 4796 | C | ALA | B | 1063 | −73.318 | −0.319 | 12.413 | 1.00 | 54.31 | C |
| ATOM | 4797 | O | ALA | B | 1063 | −74.425 | −0.102 | 12.861 | 1.00 | 53.17 | O |
| ATOM | 4798 | N | GLU | B | 1064 | −72.580 | 0.611 | 11.829 | 1.00 | 54.70 | N |
| ATOM | 4799 | CA | GLU | B | 1064 | −73.049 | 1.967 | 11.632 | 1.00 | 57.87 | C |
| ATOM | 4800 | CB | GLU | B | 1064 | −71.948 | 2.863 | 11.072 | 1.00 | 57.33 | C |
| ATOM | 4801 | CG | GLU | B | 1064 | −72.431 | 4.276 | 10.810 | 1.00 | 56.77 | C |
| ATOM | 4802 | CD | GLU | B | 1064 | −71.303 | 5.254 | 10.587 | 1.00 | 61.75 | C |
| ATOM | 4803 | OE1 | GLU | B | 1064 | −70.261 | 5.118 | 11.256 | 1.00 | 56.01 | O |
| ATOM | 4804 | OE2 | GLU | B | 1064 | −71.458 | 6.162 | 9.745 | 1.00 | 70.97 | O |
| ATOM | 4805 | C | GLU | B | 1064 | −74.316 | 2.082 | 10.773 | 1.00 | 60.84 | C |
| ATOM | 4806 | O | GLU | B | 1064 | −75.170 | 2.882 | 11.067 | 1.00 | 60.04 | O |
| ATOM | 4807 | N | LYS | B | 1065 | −74.478 | 1.224 | 9.755 | 1.00 | 62.90 | N |
| ATOM | 4808 | CA | LYS | B | 1065 | −75.725 | 1.159 | 8.990 | 1.00 | 56.79 | C |
| ATOM | 4809 | CB | LYS | B | 1065 | −75.646 | 0.118 | 7.870 | 1.00 | 60.32 | C |
| ATOM | 4810 | CG | LYS | B | 1065 | −74.767 | 0.553 | 6.705 | 1.00 | 72.81 | C |
| ATOM | 4811 | CD | LYS | B | 1065 | −74.693 | −0.504 | 5.614 | 1.00 | 82.90 | C |
| ATOM | 4812 | CE | LYS | B | 1065 | −73.652 | −0.137 | 4.562 | 1.00 | 91.93 | C |
| ATOM | 4813 | NZ | LYS | B | 1065 | −73.464 | −1.216 | 3.550 | 1.00 | 91.33 | N |
| ATOM | 4814 | C | LYS | B | 1065 | −76.923 | 0.913 | 9.888 | 1.00 | 58.00 | C |
| ATOM | 4815 | O | LYS | B | 1065 | −77.889 | 1.640 | 9.840 | 1.00 | 58.66 | O |
| ATOM | 4816 | N | LEU | B | 1066 | −76.826 | −0.115 | 10.740 | 1.00 | 53.67 | N |
| ATOM | 4817 | CA | LEU | B | 1066 | −77.908 | −0.480 | 11.640 | 1.00 | 51.85 | C |
| ATOM | 4818 | CB | LEU | B | 1066 | −77.595 | −1.804 | 12.335 | 1.00 | 45.28 | C |
| ATOM | 4819 | CG | LEU | B | 1066 | −77.435 | −3.054 | 11.474 | 1.00 | 51.51 | C |
| ATOM | 4820 | CD1 | LEU | B | 1066 | −76.830 | −4.178 | 12.300 | 1.00 | 56.13 | C |
| ATOM | 4821 | CD2 | LEU | B | 1066 | −78.771 | −3.481 | 10.893 | 1.00 | 54.16 | C |
| ATOM | 4822 | C | LEU | B | 1066 | −78.101 | 0.596 | 12.694 | 1.00 | 53.35 | C |
| ATOM | 4823 | O | LEU | B | 1066 | −79.214 | 0.885 | 13.086 | 1.00 | 54.56 | O |
| ATOM | 4824 | N | PHE | B | 1067 | −77.004 | 1.235 | 13.097 | 1.00 | 52.09 | N |
| ATOM | 4825 | CA | PHE | B | 1067 | −77.058 | 2.344 | 14.010 | 1.00 | 48.56 | C |
| ATOM | 4826 | CB | PHE | B | 1067 | −75.674 | 2.830 | 14.424 | 1.00 | 49.09 | C |
| ATOM | 4827 | CG | PHE | B | 1067 | −75.717 | 3.884 | 15.487 | 1.00 | 40.86 | C |
| ATOM | 4828 | CD1 | PHE | B | 1067 | −75.898 | 3.534 | 16.810 | 1.00 | 37.96 | C |
| ATOM | 4829 | CE1 | PHE | B | 1067 | −75.951 | 4.495 | 17.786 | 1.00 | 39.88 | C |
| ATOM | 4830 | CZ | PHE | B | 1067 | −75.832 | 5.829 | 17.450 | 1.00 | 39.95 | C |
| ATOM | 4831 | CE2 | PHE | B | 1067 | −75.662 | 6.193 | 16.136 | 1.00 | 33.44 | C |
| ATOM | 4832 | CD2 | PHE | B | 1067 | −75.611 | 5.225 | 15.160 | 1.00 | 38.25 | C |
| ATOM | 4833 | C | PHE | B | 1067 | −77.873 | 3.485 | 13.452 | 1.00 | 47.60 | C |
| ATOM | 4834 | O | PHE | B | 1067 | −78.706 | 4.031 | 14.116 | 1.00 | 53.15 | O |
| ATOM | 4835 | N | ASN | B | 1068 | −77.658 | 3.820 | 12.208 | 1.00 | 46.19 | N |
| ATOM | 4836 | CA | ASN | B | 1068 | −78.428 | 4.862 | 11.551 | 1.00 | 50.23 | C |
| ATOM | 4837 | CB | ASN | B | 1068 | −77.830 | 5.190 | 10.187 | 1.00 | 54.26 | C |
| ATOM | 4838 | CG | ASN | B | 1068 | −76.544 | 5.974 | 10.307 | 1.00 | 58.82 | C |
| ATOM | 4839 | OD1 | ASN | B | 1068 | −75.467 | 5.489 | 9.961 | 1.00 | 63.52 | O |
| ATOM | 4840 | ND2 | ASN | B | 1068 | −76.649 | 7.196 | 10.817 | 1.00 | 68.83 | N |
| ATOM | 4841 | C | ASN | B | 1068 | −79.917 | 4.520 | 11.449 | 1.00 | 56.19 | C |
| ATOM | 4842 | O | ASN | B | 1068 | −80.762 | 5.367 | 11.644 | 1.00 | 52.43 | O |
| ATOM | 4843 | N | GLN | B | 1069 | −80.237 | 3.264 | 11.156 | 1.00 | 55.11 | N |
| ATOM | 4844 | CA | GLN | B | 1069 | −81.623 | 2.826 | 11.107 | 1.00 | 55.41 | C |
| ATOM | 4845 | CB | GLN | B | 1069 | −81.708 | 1.381 | 10.612 | 1.00 | 57.78 | C |
| ATOM | 4846 | CG | GLN | B | 1069 | −81.189 | 1.199 | 9.188 | 1.00 | 60.30 | C |
| ATOM | 4847 | CD | GLN | B | 1069 | −81.218 | −0.247 | 8.733 | 1.00 | 67.35 | C |
| ATOM | 4848 | OE1 | GLN | B | 1069 | −81.836 | −1.097 | 9.375 | 1.00 | 71.43 | O |
| ATOM | 4849 | NE2 | GLN | B | 1069 | −80.541 | −0.535 | 7.623 | 1.00 | 64.34 | N |
| ATOM | 4850 | C | GLN | B | 1069 | −82.335 | 2.987 | 12.464 | 1.00 | 52.27 | C |
| ATOM | 4851 | O | GLN | B | 1069 | −83.448 | 3.470 | 12.547 | 1.00 | 58.69 | O |
| ATOM | 4852 | N | ASP | B | 1070 | −81.647 | 2.631 | 13.533 | 1.00 | 47.01 | N |
| ATOM | 4853 | CA | ASP | B | 1070 | −82.132 | 2.848 | 14.873 | 1.00 | 48.07 | C |
| ATOM | 4854 | CB | ASP | B | 1070 | −81.230 | 2.141 | 15.888 | 1.00 | 47.40 | C |
| ATOM | 4855 | CG | ASP | B | 1070 | −81.266 | 0.634 | 15.748 | 1.00 | 51.14 | C |
| ATOM | 4856 | OD1 | ASP | B | 1070 | −82.355 | 0.084 | 15.476 | 1.00 | 54.21 | O |
| ATOM | 4857 | OD2 | ASP | B | 1070 | −80.203 | −0.002 | 15.903 | 1.00 | 55.07 | O |
| ATOM | 4858 | C | ASP | B | 1070 | −82.245 | 4.330 | 15.237 | 1.00 | 45.77 | C |
| ATOM | 4859 | O | ASP | B | 1070 | −83.164 | 4.727 | 15.920 | 1.00 | 50.05 | O |
| ATOM | 4860 | N | VAL | B | 1071 | −81.301 | 5.156 | 14.780 | 1.00 | 46.34 | N |
| ATOM | 4861 | CA | VAL | B | 1071 | −81.380 | 6.609 | 14.973 | 1.00 | 48.36 | C |
| ATOM | 4862 | CB | VAL | B | 1071 | −80.114 | 7.349 | 14.476 | 1.00 | 44.30 | C |
| ATOM | 4863 | CG1 | VAL | B | 1071 | −80.292 | 8.857 | 14.594 | 1.00 | 32.58 | C |
| ATOM | 4864 | CG2 | VAL | B | 1071 | −78.909 | 6.917 | 15.274 | 1.00 | 47.35 | C |
| ATOM | 4865 | C | VAL | B | 1071 | −82.603 | 7.185 | 14.293 | 1.00 | 47.30 | C |
| ATOM | 4866 | O | VAL | B | 1071 | −83.306 | 7.983 | 14.847 | 1.00 | 45.11 | O |
| ATOM | 4867 | N | ASP | B | 1072 | −82.870 | 6.729 | 13.079 | 1.00 | 54.98 | N |
| ATOM | 4868 | CA | ASP | B | 1072 | −84.048 | 7.131 | 12.324 | 1.00 | 54.11 | C |
| ATOM | 4869 | CB | ASP | B | 1072 | −84.022 | 6.524 | 10.917 | 1.00 | 57.19 | C |
| ATOM | 4870 | CG | ASP | B | 1072 | −82.983 | 7.168 | 10.017 | 1.00 | 63.14 | C |
| ATOM | 4871 | OD1 | ASP | B | 1072 | −82.620 | 8.341 | 10.258 | 1.00 | 58.03 | O |
| ATOM | 4872 | OD2 | ASP | B | 1072 | −82.538 | 6.498 | 9.060 | 1.00 | 67.23 | O |
| ATOM | 4873 | C | ASP | B | 1072 | −85.331 | 6.712 | 13.031 | 1.00 | 54.83 | C |
| ATOM | 4874 | O | ASP | B | 1072 | −86.243 | 7.494 | 13.186 | 1.00 | 52.90 | O |
| ATOM | 4875 | N | ALA | B | 1073 | −85.354 | 5.479 | 13.526 | 1.00 | 52.87 | N |
| ATOM | 4876 | CA | ALA | B | 1073 | −86.473 | 4.980 | 14.307 | 1.00 | 53.29 | C |
| ATOM | 4877 | CB | ALA | B | 1073 | −86.245 | 3.534 | 14.679 | 1.00 | 55.21 | C |
| ATOM | 4878 | C | ALA | B | 1073 | −86.714 | 5.809 | 15.552 | 1.00 | 51.84 | C |
| ATOM | 4879 | O | ALA | B | 1073 | −87.823 | 6.165 | 15.880 | 1.00 | 59.31 | O |
| ATOM | 4880 | N | ALA | B | 1074 | −85.626 | 6.173 | 16.201 | 1.00 | 48.21 | N |
| ATOM | 4881 | CA | ALA | B | 1074 | −85.632 | 7.046 | 17.344 | 1.00 | 44.10 | C |
| ATOM | 4882 | CB | ALA | B | 1074 | −84.234 | 7.137 | 17.951 | 1.00 | 41.16 | C |
| ATOM | 4883 | C | ALA | B | 1074 | −86.148 | 8.424 | 16.990 | 1.00 | 44.91 | C |
| ATOM | 4884 | O | ALA | B | 1074 | −86.936 | 8.996 | 17.706 | 1.00 | 53.32 | O |
| ATOM | 4885 | N | VAL | B | 1075 | −85.705 | 8.973 | 15.876 | 1.00 | 44.15 | N |
| ATOM | 4886 | CA | VAL | B | 1075 | −86.165 | 10.281 | 15.464 | 1.00 | 50.23 | C |
| ATOM | 4887 | CB | VAL | B | 1075 | −85.334 | 10.832 | 14.278 | 1.00 | 46.17 | C |
| ATOM | 4888 | CG1 | VAL | B | 1075 | −85.939 | 12.120 | 13.744 | 1.00 | 45.31 | C |
| ATOM | 4889 | CG2 | VAL | B | 1075 | −83.898 | 11.073 | 14.705 | 1.00 | 49.10 | C |
| ATOM | 4890 | C | VAL | B | 1075 | −87.659 | 10.274 | 15.098 | 1.00 | 54.76 | C |
| ATOM | 4891 | O | VAL | B | 1075 | −88.388 | 11.195 | 15.422 | 1.00 | 55.86 | O |
| ATOM | 4892 | N | ARG | B | 1076 | −88.116 | 9.208 | 14.446 | 1.00 | 50.22 | N |
| ATOM | 4893 | CA | ARG | B | 1076 | −89.526 | 9.058 | 14.113 | 1.00 | 52.55 | C |
| ATOM | 4894 | CB | ARG | B | 1076 | −89.762 | 7.785 | 13.290 | 1.00 | 49.39 | C |
| ATOM | 4895 | CG | ARG | B | 1076 | −89.216 | 7.859 | 11.876 | 1.00 | 59.39 | C |
| ATOM | 4896 | CD | ARG | B | 1076 | −89.628 | 6.659 | 11.047 | 1.00 | 71.06 | C |
| ATOM | 4897 | NE | ARG | B | 1076 | −88.474 | 5.931 | 10.528 | 1.00 | 63.34 | N |
| ATOM | 4898 | CZ | ARG | B | 1076 | −88.136 | 4.701 | 10.898 | 1.00 | 57.79 | C |
| ATOM | 4899 | NH1 | ARG | B | 1076 | −88.864 | 4.042 | 11.796 | 1.00 | 56.14 | N |
| ATOM | 4900 | NH2 | ARG | B | 1076 | −87.070 | 4.129 | 10.361 | 1.00 | 56.25 | N |
| ATOM | 4901 | C | ARG | B | 1076 | −90.445 | 9.059 | 15.333 | 1.00 | 53.55 | C |
| ATOM | 4902 | O | ARG | B | 1076 | −91.442 | 9.760 | 15.384 | 1.00 | 51.56 | O |
| ATOM | 4903 | N | GLY | B | 1077 | −90.074 | 8.271 | 16.326 | 1.00 | 50.45 | N |
| ATOM | 4904 | CA | GLY | B | 1077 | −90.738 | 8.217 | 17.590 | 1.00 | 48.49 | C |
| ATOM | 4905 | C | GLY | B | 1077 | −90.735 | 9.495 | 18.322 | 1.00 | 46.05 | C |
| ATOM | 4906 | O | GLY | B | 1077 | −91.724 | 9.860 | 18.919 | 1.00 | 45.73 | O |
| ATOM | 4907 | N | ILE | B | 1078 | −89.605 | 10.181 | 18.316 | 1.00 | 47.14 | N |
| ATOM | 4908 | CA | ILE | B | 1078 | −89.511 | 11.497 | 18.906 | 1.00 | 47.23 | C |
| ATOM | 4909 | CB | ILE | B | 1078 | −88.080 | 12.054 | 18.805 | 1.00 | 38.88 | C |
| ATOM | 4910 | CG1 | ILE | B | 1078 | −87.174 | 11.311 | 19.785 | 1.00 | 36.80 | C |
| ATOM | 4911 | CD1 | ILE | B | 1078 | −85.727 | 11.737 | 19.715 | 1.00 | 49.87 | C |
| ATOM | 4912 | CG2 | ILE | B | 1078 | −88.049 | 13.543 | 19.109 | 1.00 | 42.17 | C |
| ATOM | 4913 | C | ILE | B | 1078 | −90.516 | 12.479 | 18.319 | 1.00 | 50.59 | C |
| ATOM | 4914 | O | ILE | B | 1078 | −91.184 | 13.206 | 19.019 | 1.00 | 48.77 | O |
| ATOM | 4915 | N | LEU | B | 1079 | −90.617 | 12.497 | 17.011 | 1.00 | 47.58 | N |
| ATOM | 4916 | CA | LEU | B | 1079 | −91.498 | 13.428 | 16.340 | 1.00 | 44.42 | C |
| ATOM | 4917 | CB | LEU | B | 1079 | −91.197 | 13.474 | 14.842 | 1.00 | 51.76 | C |
| ATOM | 4918 | CG | LEU | B | 1079 | −89.806 | 14.045 | 14.545 | 1.00 | 47.18 | C |
| ATOM | 4919 | CD1 | LEU | B | 1079 | −89.483 | 13.998 | 13.062 | 1.00 | 36.57 | C |
| ATOM | 4920 | CD2 | LEU | B | 1079 | −89.687 | 15.469 | 15.081 | 1.00 | 49.04 | C |
| ATOM | 4921 | C | LEU | B | 1079 | −92.974 | 13.146 | 16.614 | 1.00 | 52.31 | C |
| ATOM | 4922 | O | LEU | B | 1079 | −93.746 | 14.047 | 16.896 | 1.00 | 60.14 | O |
| ATOM | 4923 | N | ARG | B | 1080 | −93.330 | 11.865 | 16.631 | 1.00 | 51.64 | N |
| ATOM | 4924 | CA | ARG | B | 1080 | −94.650 | 11.404 | 17.044 | 1.00 | 47.53 | C |
| ATOM | 4925 | CB | ARG | B | 1080 | −94.740 | 9.883 | 16.927 | 1.00 | 50.78 | C |
| ATOM | 4926 | CG | ARG | B | 1080 | −94.694 | 9.371 | 15.500 | 1.00 | 57.06 | C |
| ATOM | 4927 | CD | ARG | B | 1080 | −96.105 | 9.128 | 14.986 | 1.00 | 60.10 | C |
| ATOM | 4928 | NE | ARG | B | 1080 | −96.835 | 8.211 | 15.865 | 1.00 | 66.32 | N |
| ATOM | 4929 | CZ | ARG | B | 1080 | −98.162 | 8.138 | 15.945 | 1.00 | 71.74 | C |
| ATOM | 4930 | NH1 | ARG | B | 1080 | −98.915 | 8.933 | 15.198 | 1.00 | 73.15 | N |
| ATOM | 4931 | NH2 | ARG | B | 1080 | −98.740 | 7.278 | 16.778 | 1.00 | 63.50 | N |
| ATOM | 4932 | C | ARG | B | 1080 | −95.077 | 11.862 | 18.456 | 1.00 | 48.41 | C |
| ATOM | 4933 | O | ARG | B | 1080 | −96.220 | 12.233 | 18.664 | 1.00 | 58.65 | O |
| ATOM | 4934 | N | ASN | B | 1081 | −94.156 | 11.812 | 19.424 | 1.00 | 49.47 | N |
| ATOM | 4935 | CA | ASN | B | 1081 | −94.394 | 12.273 | 20.815 | 1.00 | 45.07 | C |
| ATOM | 4936 | CB | ASN | B | 1081 | −93.255 | 11.802 | 21.726 | 1.00 | 45.69 | C |
| ATOM | 4937 | CG | ASN | B | 1081 | −93.636 | 11.818 | 23.194 | 1.00 | 41.24 | C |
| ATOM | 4938 | OD1 | ASN | B | 1081 | −93.845 | 12.879 | 23.779 | 1.00 | 46.70 | O |
| ATOM | 4939 | ND2 | ASN | B | 1081 | −93.723 | 10.637 | 23.799 | 1.00 | 36.47 | N |
| ATOM | 4940 | C | ASN | B | 1081 | −94.582 | 13.797 | 20.961 | 1.00 | 47.16 | C |
| ATOM | 4941 | O | ASN | B | 1081 | −93.692 | 14.583 | 20.712 | 1.00 | 48.80 | O |
| ATOM | 4942 | N | ALA | B | 1082 | −95.761 | 14.182 | 21.417 | 1.00 | 51.67 | N |
| ATOM | 4943 | CA | ALA | B | 1082 | −96.185 | 15.563 | 21.538 | 1.00 | 47.20 | C |
| ATOM | 4944 | CB | ALA | B | 1082 | −97.642 | 15.628 | 21.981 | 1.00 | 44.62 | C |
| ATOM | 4945 | C | ALA | B | 1082 | −95.335 | 16.442 | 22.440 | 1.00 | 44.81 | C |
| ATOM | 4946 | O | ALA | B | 1082 | −95.397 | 17.650 | 22.321 | 1.00 | 44.89 | O |
| ATOM | 4947 | N | LYS | B | 1083 | −94.613 | 15.848 | 23.392 | 1.00 | 42.00 | N |
| ATOM | 4948 | CA | LYS | B | 1083 | −93.840 | 16.634 | 24.338 | 1.00 | 44.54 | C |
| ATOM | 4949 | CB | LYS | B | 1083 | −93.931 | 16.015 | 25.739 | 1.00 | 45.11 | C |
| ATOM | 4950 | CG | LYS | B | 1083 | −95.321 | 16.046 | 26.352 | 1.00 | 61.64 | C |
| ATOM | 4951 | CD | LYS | B | 1083 | −95.405 | 15.240 | 27.644 | 1.00 | 55.16 | C |
| ATOM | 4952 | CE | LYS | B | 1083 | −95.775 | 13.787 | 27.377 | 1.00 | 47.26 | C |
| ATOM | 4953 | NZ | LYS | B | 1083 | −96.164 | 13.078 | 28.630 | 1.00 | 40.95 | N |
| ATOM | 4954 | C | LYS | B | 1083 | −92.371 | 16.714 | 23.930 | 1.00 | 46.45 | C |
| ATOM | 4955 | O | LYS | B | 1083 | −91.604 | 17.467 | 24.508 | 1.00 | 40.39 | O |
| ATOM | 4956 | N | LEU | B | 1084 | −92.004 | 15.904 | 22.939 | 1.00 | 42.97 | N |
| ATOM | 4957 | CA | LEU | B | 1084 | −90.634 | 15.772 | 22.500 | 1.00 | 44.80 | C |
| ATOM | 4958 | CB | LEU | B | 1084 | −90.237 | 14.298 | 22.365 | 1.00 | 37.78 | C |
| ATOM | 4959 | CG | LEU | B | 1084 | −90.139 | 13.471 | 23.649 | 1.00 | 40.38 | C |
| ATOM | 4960 | CD1 | LEU | B | 1084 | −89.749 | 12.037 | 23.325 | 1.00 | 41.78 | C |
| ATOM | 4961 | CD2 | LEU | B | 1084 | −89.154 | 14.083 | 24.639 | 1.00 | 40.75 | C |
| ATOM | 4962 | C | LEU | B | 1084 | −90.411 | 16.488 | 21.202 | 1.00 | 51.95 | C |
| ATOM | 4963 | O | LEU | B | 1084 | −89.331 | 16.978 | 20.959 | 1.00 | 52.14 | O |
| ATOM | 4964 | N | LYS | B | 1085 | −91.442 | 16.561 | 20.365 | 1.00 | 48.25 | N |
| ATOM | 4965 | CA | LYS | B | 1085 | −91.314 | 17.217 | 19.069 | 1.00 | 46.71 | C |
| ATOM | 4966 | CB | LYS | B | 1085 | −92.573 | 17.044 | 18.213 | 1.00 | 46.98 | C |
| ATOM | 4967 | CG | LYS | B | 1085 | −92.474 | 17.734 | 16.859 | 1.00 | 50.91 | C |
| ATOM | 4968 | CD | LYS | B | 1085 | −93.655 | 17.409 | 15.961 | 1.00 | 51.34 | C |
| ATOM | 4969 | CE | LYS | B | 1085 | −93.445 | 17.966 | 14.557 | 1.00 | 53.03 | C |
| ATOM | 4970 | NZ | LYS | B | 1085 | −93.290 | 19.449 | 14.540 | 1.00 | 49.35 | N |
| ATOM | 4971 | C | LYS | B | 1085 | −90.953 | 18.698 | 19.199 | 1.00 | 48.88 | C |
| ATOM | 4972 | O | LYS | B | 1085 | −90.055 | 19.123 | 18.498 | 1.00 | 50.71 | O |
| ATOM | 4973 | N | PRO | B | 1086 | −91.542 | 19.593 | 19.997 | 1.00 | 50.42 | N |
| ATOM | 4974 | CA | PRO | B | 1086 | −91.124 | 20.996 | 20.043 | 1.00 | 58.38 | C |
| ATOM | 4975 | CB | PRO | B | 1086 | −92.012 | 21.584 | 21.139 | 1.00 | 52.24 | C |
| ATOM | 4976 | CG | PRO | B | 1086 | −93.246 | 20.772 | 21.071 | 1.00 | 50.03 | C |
| ATOM | 4977 | CD | PRO | B | 1086 | −92.765 | 19.377 | 20.783 | 1.00 | 49.22 | C |
| ATOM | 4978 | C | PRO | B | 1086 | −89.638 | 21.144 | 20.414 | 1.00 | 59.09 | C |
| ATOM | 4979 | O | PRO | B | 1086 | −88.895 | 21.913 | 19.807 | 1.00 | 58.82 | O |
| ATOM | 4980 | N | VAL | B | 1087 | −89.228 | 20.389 | 21.410 | 1.00 | 54.18 | N |
| ATOM | 4981 | CA | VAL | B | 1087 | −87.871 | 20.429 | 21.861 | 1.00 | 53.23 | C |
| ATOM | 4982 | CB | VAL | B | 1087 | −87.654 | 19.538 | 23.095 | 1.00 | 52.53 | C |
| ATOM | 4983 | CG1 | VAL | B | 1087 | −86.194 | 19.570 | 23.514 | 1.00 | 61.86 | C |
| ATOM | 4984 | CG2 | VAL | B | 1087 | −88.546 | 19.991 | 24.232 | 1.00 | 53.65 | C |
| ATOM | 4985 | C | VAL | B | 1087 | −86.889 | 20.017 | 20.764 | 1.00 | 51.26 | C |
| ATOM | 4986 | O | VAL | B | 1087 | −85.929 | 20.692 | 20.506 | 1.00 | 55.00 | O |
| ATOM | 4987 | N | TYR | B | 1088 | −87.149 | 18.904 | 20.107 | 1.00 | 44.01 | N |
| ATOM | 4988 | CA | TYR | B | 1088 | −86.265 | 18.391 | 19.098 | 1.00 | 39.48 | C |
| ATOM | 4989 | CB | TYR | B | 1088 | −86.805 | 17.067 | 18.553 | 1.00 | 42.13 | C |
| ATOM | 4990 | CG | TYR | B | 1088 | −85.873 | 16.353 | 17.599 | 1.00 | 45.38 | C |
| ATOM | 4991 | CD1 | TYR | B | 1088 | −84.971 | 15.398 | 18.057 | 1.00 | 45.61 | C |
| ATOM | 4992 | CE1 | TYR | B | 1088 | −84.118 | 14.740 | 17.185 | 1.00 | 42.99 | C |
| ATOM | 4993 | CZ | TYR | B | 1088 | −84.159 | 15.036 | 15.841 | 1.00 | 45.29 | C |
| ATOM | 4994 | OH | TYR | B | 1088 | −83.313 | 14.390 | 14.973 | 1.00 | 48.46 | O |
| ATOM | 4995 | CE2 | TYR | B | 1088 | −85.043 | 15.979 | 15.361 | 1.00 | 50.55 | C |
| ATOM | 4996 | CD2 | TYR | B | 1088 | −85.896 | 16.629 | 16.238 | 1.00 | 48.46 | C |
| ATOM | 4997 | C | TYR | B | 1088 | −86.078 | 19.380 | 17.979 | 1.00 | 45.38 | C |
| ATOM | 4998 | O | TYR | B | 1088 | −84.994 | 19.549 | 17.473 | 1.00 | 53.14 | O |
| ATOM | 4999 | N | ASP | B | 1089 | −87.149 | 20.008 | 17.550 | 1.00 | 51.44 | N |
| ATOM | 5000 | CA | ASP | B | 1089 | −87.059 | 20.908 | 16.413 | 1.00 | 49.84 | C |
| ATOM | 5001 | CB | ASP | B | 1089 | −88.455 | 21.373 | 15.986 | 1.00 | 55.70 | C |
| ATOM | 5002 | CG | ASP | B | 1089 | −89.291 | 20.258 | 15.378 | 1.00 | 61.38 | C |
| ATOM | 5003 | OD1 | ASP | B | 1089 | −88.910 | 19.072 | 15.497 | 1.00 | 59.10 | O |
| ATOM | 5004 | OD2 | ASP | B | 1089 | −90.343 | 20.573 | 14.784 | 1.00 | 66.50 | O |
| ATOM | 5005 | C | ASP | B | 1089 | −86.203 | 22.115 | 16.731 | 1.00 | 51.84 | C |
| ATOM | 5006 | O | ASP | B | 1089 | −85.495 | 22.599 | 15.861 | 1.00 | 60.16 | O |
| ATOM | 5007 | N | SER | B | 1090 | −86.354 | 22.666 | 17.945 | 1.00 | 47.47 | N |
| ATOM | 5008 | CA | SER | B | 1090 | −85.591 | 23.849 | 18.318 | 1.00 | 44.62 | C |
| ATOM | 5009 | CB | SER | B | 1090 | −86.090 | 24.413 | 19.651 | 1.00 | 53.31 | C |
| ATOM | 5010 | OG | SER | B | 1090 | −85.878 | 23.498 | 20.710 | 1.00 | 53.86 | O |
| ATOM | 5011 | C | SER | B | 1090 | −84.087 | 23.631 | 18.369 | 1.00 | 48.62 | C |
| ATOM | 5012 | O | SER | B | 1090 | −83.313 | 24.553 | 18.166 | 1.00 | 57.47 | O |
| ATOM | 5013 | N | LEU | B | 1091 | −83.707 | 22.423 | 18.774 | 1.00 | 51.01 | N |
| ATOM | 5014 | CA | LEU | B | 1091 | −82.323 | 22.052 | 18.986 | 1.00 | 46.70 | C |
| ATOM | 5015 | CB | LEU | B | 1091 | −82.237 | 20.733 | 19.757 | 1.00 | 43.99 | C |
| ATOM | 5016 | CG | LEU | B | 1091 | −82.675 | 20.786 | 21.219 | 1.00 | 46.38 | C |
| ATOM | 5017 | CD1 | LEU | B | 1091 | −82.598 | 19.405 | 21.843 | 1.00 | 45.91 | C |
| ATOM | 5018 | CD2 | LEU | B | 1091 | −81.828 | 21.780 | 22.000 | 1.00 | 45.87 | C |
| ATOM | 5019 | C | LEU | B | 1091 | −81.477 | 21.958 | 17.720 | 1.00 | 51.17 | C |
| ATOM | 5020 | O | LEU | B | 1091 | −81.913 | 21.500 | 16.676 | 1.00 | 42.01 | O |
| ATOM | 5021 | N | ASP | B | 1092 | −80.202 | 22.312 | 17.902 | 1.00 | 56.26 | N |
| ATOM | 5022 | CA | ASP | B | 1092 | −79.157 | 22.138 | 16.905 | 1.00 | 50.39 | C |
| ATOM | 5023 | CB | ASP | B | 1092 | −77.957 | 23.012 | 17.259 | 1.00 | 47.03 | C |
| ATOM | 5024 | CG | ASP | B | 1092 | −77.353 | 22.645 | 18.597 | 1.00 | 52.41 | C |
| ATOM | 5025 | OD1 | ASP | B | 1092 | −78.036 | 22.845 | 19.625 | 1.00 | 53.46 | O |
| ATOM | 5026 | OD2 | ASP | B | 1092 | −76.200 | 22.163 | 18.622 | 1.00 | 54.35 | O |
| ATOM | 5027 | C | ASP | B | 1092 | −78.717 | 20.688 | 16.816 | 1.00 | 47.09 | C |
| ATOM | 5028 | O | ASP | B | 1092 | −79.057 | 19.912 | 17.672 | 1.00 | 51.76 | O |
| ATOM | 5029 | N | ALA | B | 1093 | −77.927 | 20.357 | 15.796 | 1.00 | 46.75 | N |
| ATOM | 5030 | CA | ALA | B | 1093 | −77.477 | 18.991 | 15.519 | 1.00 | 48.14 | C |
| ATOM | 5031 | CB | ALA | B | 1093 | −76.514 | 18.983 | 14.338 | 1.00 | 52.36 | C |
| ATOM | 5032 | C | ALA | B | 1093 | −76.855 | 18.281 | 16.718 | 1.00 | 50.13 | C |
| ATOM | 5033 | O | ALA | B | 1093 | −77.190 | 17.159 | 17.037 | 1.00 | 53.50 | O |
| ATOM | 5034 | N | VAL | B | 1094 | −75.937 | 18.941 | 17.392 | 1.00 | 48.64 | N |
| ATOM | 5035 | CA | VAL | B | 1094 | −75.192 | 18.292 | 18.445 | 1.00 | 48.59 | C |
| ATOM | 5036 | CB | VAL | B | 1094 | −74.027 | 19.179 | 18.951 | 1.00 | 44.97 | C |
| ATOM | 5037 | CG1 | VAL | B | 1094 | −73.152 | 18.408 | 19.925 | 1.00 | 37.06 | C |
| ATOM | 5038 | CG2 | VAL | B | 1094 | −73.197 | 19.689 | 17.784 | 1.00 | 44.06 | C |
| ATOM | 5039 | C | VAL | B | 1094 | −76.080 | 17.922 | 19.634 | 1.00 | 46.93 | C |
| ATOM | 5040 | O | VAL | B | 1094 | −75.916 | 16.873 | 20.228 | 1.00 | 45.35 | O |
| ATOM | 5041 | N | ARG | B | 1095 | −77.061 | 18.781 | 19.934 | 1.00 | 46.98 | N |
| ATOM | 5042 | CA | ARG | B | 1095 | −77.978 | 18.558 | 21.041 | 1.00 | 44.34 | C |
| ATOM | 5043 | CB | ARG | B | 1095 | −78.547 | 19.881 | 21.565 | 1.00 | 42.67 | C |
| ATOM | 5044 | CG | ARG | B | 1095 | −77.489 | 20.775 | 22.207 | 1.00 | 48.50 | C |
| ATOM | 5045 | CD | ARG | B | 1095 | −78.108 | 21.949 | 22.930 | 1.00 | 46.15 | C |
| ATOM | 5046 | NE | ARG | B | 1095 | −77.121 | 22.766 | 23.631 | 1.00 | 41.97 | N |
| ATOM | 5047 | CZ | ARG | B | 1095 | −76.582 | 23.875 | 23.137 | 1.00 | 48.26 | C |
| ATOM | 5048 | NH1 | ARG | B | 1095 | −76.922 | 24.301 | 21.926 | 1.00 | 49.36 | N |
| ATOM | 5049 | NH2 | ARG | B | 1095 | −75.700 | 24.560 | 23.855 | 1.00 | 47.26 | N |
| ATOM | 5050 | C | ARG | B | 1095 | −79.082 | 17.575 | 20.711 | 1.00 | 42.44 | C |
| ATOM | 5051 | O | ARG | B | 1095 | −79.454 | 16.777 | 21.539 | 1.00 | 46.29 | O |
| ATOM | 5052 | N | ARG | B | 1096 | −79.553 | 17.563 | 19.474 | 1.00 | 37.63 | N |
| ATOM | 5053 | CA | ARG | B | 1096 | −80.417 | 16.499 | 19.013 | 1.00 | 37.25 | C |
| ATOM | 5054 | CB | ARG | B | 1096 | −80.731 | 16.665 | 17.523 | 1.00 | 43.40 | C |
| ATOM | 5055 | CG | ARG | B | 1096 | −81.605 | 17.868 | 17.189 | 1.00 | 44.05 | C |
| ATOM | 5056 | CD | ARG | B | 1096 | −82.012 | 17.843 | 15.723 | 1.00 | 49.51 | C |
| ATOM | 5057 | NE | ARG | B | 1096 | −82.936 | 18.921 | 15.375 | 1.00 | 55.14 | N |
| ATOM | 5058 | CZ | ARG | B | 1096 | −83.532 | 19.038 | 14.191 | 1.00 | 63.94 | C |
| ATOM | 5059 | NH1 | ARG | B | 1096 | −83.304 | 18.141 | 13.238 | 1.00 | 64.63 | N |
| ATOM | 5060 | NH2 | ARG | B | 1096 | −84.358 | 20.049 | 13.957 | 1.00 | 65.47 | N |
| ATOM | 5061 | C | ARG | B | 1096 | −79.849 | 15.103 | 19.281 | 1.00 | 40.93 | C |
| ATOM | 5062 | O | ARG | B | 1096 | −80.545 | 14.206 | 19.699 | 1.00 | 42.63 | O |
| ATOM | 5063 | N | SER | B | 1097 | −78.540 | 14.969 | 19.132 | 1.00 | 44.17 | N |
| ATOM | 5064 | CA | SER | B | 1097 | −77.814 | 13.750 | 19.463 | 1.00 | 44.07 | C |
| ATOM | 5065 | CB | SER | B | 1097 | −76.355 | 13.855 | 19.031 | 1.00 | 39.56 | C |
| ATOM | 5066 | OG | SER | B | 1097 | −76.260 | 13.911 | 17.620 | 1.00 | 45.01 | O |
| ATOM | 5067 | C | SER | B | 1097 | −77.895 | 13.332 | 20.928 | 1.00 | 41.75 | C |
| ATOM | 5068 | O | SER | B | 1097 | −78.084 | 12.180 | 21.263 | 1.00 | 39.49 | O |
| ATOM | 5069 | N | ALA | B | 1098 | −77.774 | 14.308 | 21.806 | 1.00 | 39.16 | N |
| ATOM | 5070 | CA | ALA | B | 1098 | −77.884 | 14.083 | 23.235 | 1.00 | 40.95 | C |
| ATOM | 5071 | CB | ALA | B | 1098 | −77.523 | 15.357 | 24.005 | 1.00 | 34.60 | C |
| ATOM | 5072 | C | ALA | B | 1098 | −79.273 | 13.591 | 23.642 | 1.00 | 44.44 | C |
| ATOM | 5073 | O | ALA | B | 1098 | −79.412 | 12.672 | 24.427 | 1.00 | 46.58 | O |
| ATOM | 5074 | N | LEU | B | 1099 | −80.298 | 14.185 | 23.030 | 1.00 | 39.79 | N |
| ATOM | 5075 | CA | LEU | B | 1099 | −81.680 | 13.766 | 23.191 | 1.00 | 39.87 | C |
| ATOM | 5076 | CB | LEU | B | 1099 | −82.609 | 14.718 | 22.437 | 1.00 | 43.58 | C |
| ATOM | 5077 | CG | LEU | B | 1099 | −84.098 | 14.582 | 22.745 | 1.00 | 40.39 | C |
| ATOM | 5078 | CD1 | LEU | B | 1099 | −84.332 | 14.791 | 24.224 | 1.00 | 35.40 | C |
| ATOM | 5079 | CD2 | LEU | B | 1099 | −84.896 | 15.583 | 21.934 | 1.00 | 40.90 | C |
| ATOM | 5080 | C | LEU | B | 1099 | −81.908 | 12.342 | 22.723 | 1.00 | 40.64 | C |
| ATOM | 5081 | O | LEU | B | 1099 | −82.533 | 11.547 | 23.386 | 1.00 | 40.38 | O |
| ATOM | 5082 | N | ILE | B | 1100 | −81.347 | 12.012 | 21.578 | 1.00 | 39.75 | N |
| ATOM | 5083 | CA | ILE | B | 1100 | −81.351 | 10.654 | 21.086 | 1.00 | 44.71 | C |
| ATOM | 5084 | CB | ILE | B | 1100 | −80.738 | 10.556 | 19.682 | 1.00 | 46.38 | C |
| ATOM | 5085 | CG1 | ILE | B | 1100 | −81.638 | 11.289 | 18.686 | 1.00 | 41.48 | C |
| ATOM | 5086 | CD1 | ILE | B | 1100 | −81.160 | 11.207 | 17.254 | 1.00 | 44.70 | C |
| ATOM | 5087 | CG2 | ILE | B | 1100 | −80.560 | 9.099 | 19.269 | 1.00 | 39.32 | C |
| ATOM | 5088 | C | ILE | B | 1100 | −80.685 | 9.661 | 22.066 | 1.00 | 46.07 | C |
| ATOM | 5089 | O | ILE | B | 1100 | −81.208 | 8.603 | 22.328 | 1.00 | 45.13 | O |
| ATOM | 5090 | N | ASN | B | 1101 | −79.566 | 10.041 | 22.666 | 1.00 | 42.61 | N |
| ATOM | 5091 | CA | ASN | B | 1101 | −78.861 | 9.197 | 23.624 | 1.00 | 40.71 | C |
| ATOM | 5092 | CB | ASN | B | 1101 | −77.602 | 9.931 | 24.100 | 1.00 | 44.90 | C |
| ATOM | 5093 | CG | ASN | B | 1101 | −76.575 | 9.006 | 24.734 | 1.00 | 42.83 | C |
| ATOM | 5094 | OD1 | ASN | B | 1101 | −76.777 | 8.479 | 25.829 | 1.00 | 42.64 | O |
| ATOM | 5095 | ND2 | ASN | B | 1101 | −75.450 | 8.827 | 24.052 | 1.00 | 35.62 | N |
| ATOM | 5096 | C | ASN | B | 1101 | −79.737 | 8.846 | 24.827 | 1.00 | 40.82 | C |
| ATOM | 5097 | O | ASN | B | 1101 | −79.798 | 7.701 | 25.230 | 1.00 | 44.02 | O |
| ATOM | 5098 | N | MET | B | 1102 | −80.481 | 9.828 | 25.325 | 1.00 | 40.73 | N |
| ATOM | 5099 | CA | MET | B | 1102 | −81.533 | 9.606 | 26.325 | 1.00 | 39.49 | C |
| ATOM | 5100 | CB | MET | B | 1102 | −82.148 | 10.933 | 26.777 | 1.00 | 34.50 | C |
| ATOM | 5101 | CG | MET | B | 1102 | −81.265 | 11.726 | 27.724 | 1.00 | 36.31 | C |
| ATOM | 5102 | SD | MET | B | 1102 | −81.988 | 13.296 | 28.234 | 1.00 | 40.74 | S |
| ATOM | 5103 | CE | MET | B | 1102 | −82.614 | 12.896 | 29.863 | 1.00 | 22.28 | C |
| ATOM | 5104 | C | MET | B | 1102 | −82.650 | 8.605 | 25.868 | 1.00 | 39.94 | C |
| ATOM | 5105 | O | MET | B | 1102 | −83.041 | 7.689 | 26.562 | 1.00 | 36.58 | O |
| ATOM | 5106 | N | VAL | B | 1103 | −83.121 | 8.762 | 24.650 | 1.00 | 39.95 | N |
| ATOM | 5107 | CA | VAL | B | 1103 | −84.101 | 7.859 | 24.049 | 1.00 | 43.71 | C |
| ATOM | 5108 | CB | VAL | B | 1103 | −84.557 | 8.355 | 22.655 | 1.00 | 40.75 | C |
| ATOM | 5109 | CG1 | VAL | B | 1103 | −85.431 | 7.319 | 21.967 | 1.00 | 42.64 | C |
| ATOM | 5110 | CG2 | VAL | B | 1103 | −85.306 | 9.665 | 22.789 | 1.00 | 40.36 | C |
| ATOM | 5111 | C | VAL | B | 1103 | −83.581 | 6.412 | 23.966 | 1.00 | 41.55 | C |
| ATOM | 5112 | O | VAL | B | 1103 | −84.266 | 5.452 | 24.252 | 1.00 | 42.61 | O |
| ATOM | 5113 | N | PHE | B | 1104 | −82.326 | 6.279 | 23.614 | 1.00 | 39.68 | N |
| ATOM | 5114 | CA | PHE | B | 1104 | −81.641 | 5.004 | 23.581 | 1.00 | 38.18 | C |
| ATOM | 5115 | CB | PHE | B | 1104 | −80.245 | 5.172 | 22.991 | 1.00 | 45.86 | C |
| ATOM | 5116 | CG | PHE | B | 1104 | −80.149 | 4.822 | 21.534 | 1.00 | 46.45 | C |
| ATOM | 5117 | CD1 | PHE | B | 1104 | −80.351 | 5.785 | 20.563 | 1.00 | 43.22 | C |
| ATOM | 5118 | CE1 | PHE | B | 1104 | −80.243 | 5.468 | 19.221 | 1.00 | 52.36 | C |
| ATOM | 5119 | CZ | PHE | B | 1104 | −79.927 | 4.176 | 18.835 | 1.00 | 52.28 | C |
| ATOM | 5120 | CE2P | HE | B | 1104 | −79.717 | 3.205 | 19.793 | 1.00 | 50.36 | C |
| ATOM | 5121 | CD2 | PHE | B | 1104 | −79.825 | 3.530 | 21.135 | 1.00 | 48.93 | C |
| ATOM | 5122 | C | PHE | B | 1104 | −81.547 | 4.370 | 24.988 | 1.00 | 39.21 | C |
| ATOM | 5123 | O | PHE | B | 1104 | −81.722 | 3.179 | 25.164 | 1.00 | 45.73 | O |
| ATOM | 5124 | N | GLN | B | 1105 | −81.281 | 5.197 | 25.991 | 1.00 | 39.19 | N |
| ATOM | 5125 | CA | GLN | B | 1105 | −81.193 | 4.750 | 27.368 | 1.00 | 39.02 | C |
| ATOM | 5126 | CB | GLN | B | 1105 | −80.228 | 5.656 | 28.138 | 1.00 | 34.43 | C |
| ATOM | 5127 | CG | GLN | B | 1105 | −79.868 | 5.135 | 29.510 | 1.00 | 35.43 | C |
| ATOM | 5128 | CD | GLN | B | 1105 | −78.796 | 5.965 | 30.185 | 1.00 | 40.36 | C |
| ATOM | 5129 | OE1 | GLN | B | 1105 | −78.402 | 7.022 | 29.688 | 1.00 | 41.71 | O |
| ATOM | 5130 | NE2 | GLN | B | 1105 | −78.314 | 5.487 | 31.326 | 1.00 | 40.04 | N |
| ATOM | 5131 | C | GLN | B | 1105 | −82.490 | 4.578 | 28.179 | 1.00 | 42.17 | C |
| ATOM | 5132 | O | GLN | B | 1105 | −82.704 | 3.553 | 28.803 | 1.00 | 38.66 | O |
| ATOM | 5133 | N | MET | B | 1106 | −83.330 | 5.610 | 28.210 | 1.00 | 40.10 | N |
| ATOM | 5134 | CA | MET | B | 1106 | −84.556 | 5.595 | 28.990 | 1.00 | 37.18 | C |
| ATOM | 5135 | CB | MET | B | 1106 | −84.771 | 6.953 | 29.644 | 1.00 | 34.12 | C |
| ATOM | 5136 | CG | MET | B | 1106 | −83.570 | 7.467 | 30.399 | 1.00 | 40.20 | C |
| ATOM | 5137 | SD | MET | B | 1106 | −83.960 | 9.047 | 31.147 | 1.00 | 61.18 | S |
| ATOM | 5138 | CE | MET | B | 1106 | −84.660 | 9.864 | 29.727 | 1.00 | 39.36 | C |
| ATOM | 5139 | C | MET | B | 1106 | −85.772 | 5.258 | 28.117 | 1.00 | 38.83 | C |
| ATOM | 5140 | O | MET | B | 1106 | −86.760 | 4.738 | 28.584 | 1.00 | 40.07 | O |
| ATOM | 5141 | N | GLY | B | 1107 | −85.743 | 5.664 | 26.877 | 1.00 | 40.10 | N |
| ATOM | 5142 | CA | GLY | B | 1107 | −86.916 | 5.550 | 26.012 | 1.00 | 39.55 | C |
| ATOM | 5143 | C | GLY | B | 1107 | −87.657 | 6.867 | 25.830 | 1.00 | 42.91 | C |
| ATOM | 5144 | O | GLY | B | 1107 | −87.468 | 7.758 | 26.620 | 1.00 | 42.27 | O |
| ATOM | 5145 | N | GLU | B | 1108 | −88.534 | 6.969 | 24.830 | 1.00 | 45.61 | N |
| ATOM | 5146 | CA | GLU | B | 1108 | −89.337 | 8.167 | 24.553 | 1.00 | 44.71 | C |
| ATOM | 5147 | CB | GLU | B | 1108 | −90.200 | 7.981 | 23.301 | 1.00 | 44.15 | C |
| ATOM | 5148 | CG | GLU | B | 1108 | −89.414 | 7.806 | 22.009 | 1.00 | 44.31 | C |
| ATOM | 5149 | CD | GLU | B | 1108 | −89.058 | 6.357 | 21.726 | 1.00 | 53.05 | C |
| ATOM | 5150 | OE1 | GLU | B | 1108 | −89.305 | 5.496 | 22.598 | 1.00 | 53.98 | O |
| ATOM | 5151 | OE2 | GLU | B | 1108 | −88.533 | 6.080 | 20.625 | 1.00 | 53.93 | O |
| ATOM | 5152 | C | GLU | B | 1108 | −90.199 | 8.608 | 25.719 | 1.00 | 42.70 | C |
| ATOM | 5153 | O | GLU | B | 1108 | −90.258 | 9.770 | 26.050 | 1.00 | 40.84 | O |
| ATOM | 5154 | N | THR | B | 1109 | −90.864 | 7.644 | 26.334 | 1.00 | 44.82 | N |
| ATOM | 5155 | CA | THR | B | 1109 | −91.660 | 7.855 | 27.540 | 1.00 | 49.75 | C |
| ATOM | 5156 | CB | THR | B | 1109 | −92.296 | 6.543 | 28.026 | 1.00 | 49.51 | C |
| ATOM | 5157 | OG1 | THR | B | 1109 | −93.109 | 5.992 | 26.984 | 1.00 | 52.43 | O |
| ATOM | 5158 | CG2 | THR | B | 1109 | −93.148 | 6.792 | 29.261 | 1.00 | 45.41 | C |
| ATOM | 5159 | C | THR | B | 1109 | −90.849 | 8.456 | 28.699 | 1.00 | 46.23 | C |
| ATOM | 5160 | O | THR | B | 1109 | −91.248 | 9.433 | 29.307 | 1.00 | 49.47 | O |
| ATOM | 5161 | N | GLY | B | 1110 | −89.670 | 7.887 | 28.940 | 1.00 | 46.45 | N |
| ATOM | 5162 | CA | GLY | B | 1110 | −88.696 | 8.335 | 29.907 | 1.00 | 43.39 | C |
| ATOM | 5163 | C | GLY | B | 1110 | −88.261 | 9.742 | 29.744 | 1.00 | 38.99 | C |
| ATOM | 5164 | O | GLY | B | 1110 | −88.285 | 10.521 | 30.669 | 1.00 | 35.99 | O |
| ATOM | 5165 | N | VAL | B | 1111 | −87.877 | 10.080 | 28.528 | 1.00 | 42.40 | N |
| ATOM | 5166 | CA | VAL | B | 1111 | −87.463 | 11.426 | 28.189 | 1.00 | 46.83 | C |
| ATOM | 5167 | CB | VAL | B | 1111 | −86.915 | 11.524 | 26.740 | 1.00 | 47.32 | C |
| ATOM | 5168 | CG1 | VAL | B | 1111 | −86.237 | 12.863 | 26.526 | 1.00 | 41.25 | C |
| ATOM | 5169 | CG2 | VAL | B | 1111 | −85.928 | 10.400 | 26.455 | 1.00 | 35.57 | C |
| ATOM | 5170 | C | VAL | B | 1111 | −88.607 | 12.404 | 28.403 | 1.00 | 47.33 | C |
| ATOM | 5171 | O | VAL | B | 1111 | −88.422 | 13.487 | 28.905 | 1.00 | 48.62 | O |
| ATOM | 5172 | N | ALA | B | 1112 | −89.806 | 12.004 | 27.986 | 1.00 | 45.22 | N |
| ATOM | 5173 | CA | ALA | B | 1112 | −91.006 | 12.848 | 28.076 | 1.00 | 49.54 | C |
| ATOM | 5174 | CB | ALA | B | 1112 | −92.206 | 12.135 | 27.465 | 1.00 | 46.99 | C |
| ATOM | 5175 | C | ALA | B | 1112 | −91.307 | 13.277 | 29.498 | 1.00 | 48.25 | C |
| ATOM | 5176 | O | ALA | B | 1112 | −91.677 | 14.403 | 29.740 | 1.00 | 54.61 | O |
| ATOM | 5177 | N | GLY | B | 1113 | −91.072 | 12.392 | 30.442 | 1.00 | 42.73 | N |
| ATOM | 5178 | CA | GLY | B | 1113 | −91.174 | 12.752 | 31.838 | 1.00 | 48.68 | C |
| ATOM | 5179 | C | GLY | B | 1113 | −90.431 | 14.018 | 32.336 | 1.00 | 51.64 | C |
| ATOM | 5180 | O | GLY | B | 1113 | −90.812 | 14.560 | 33.352 | 1.00 | 61.26 | O |
| ATOM | 5181 | N | PHE | B | 1114 | −89.397 | 14.499 | 31.661 | 1.00 | 53.16 | N |
| ATOM | 5182 | CA | PHE | B | 1114 | −88.670 | 15.699 | 32.093 | 1.00 | 62.47 | C |
| ATOM | 5183 | CB | PHE | B | 1114 | −87.206 | 15.646 | 31.636 | 1.00 | 55.47 | C |
| ATOM | 5184 | CG | PHE | B | 1114 | −86.401 | 14.555 | 32.289 | 1.00 | 51.20 | C |
| ATOM | 5185 | CD1 | PHE | B | 1114 | −85.705 | 14.795 | 33.462 | 1.00 | 50.59 | C |
| ATOM | 5186 | CE1 | PHE | B | 1114 | −84.965 | 13.794 | 34.063 | 1.00 | 45.84 | C |
| ATOM | 5187 | CZ | PHE | B | 1114 | −84.912 | 12.537 | 33.494 | 1.00 | 41.98 | C |
| ATOM | 5188 | CE2 | PHE | B | 1114 | −85.596 | 12.284 | 32.325 | 1.00 | 36.70 | C |
| ATOM | 5189 | CD2 | PHE | B | 1114 | −86.335 | 13.289 | 31.726 | 1.00 | 46.74 | C |
| ATOM | 5190 | C | PHE | B | 1114 | −89.321 | 16.981 | 31.566 | 1.00 | 66.48 | C |
| ATOM | 5191 | O | PHE | B | 1114 | −88.833 | 17.565 | 30.620 | 1.00 | 62.55 | O |
| ATOM | 5192 | N | THR | B | 1115 | −90.476 | 17.363 | 32.108 | 1.00 | 67.34 | N |
| ATOM | 5193 | CA | THR | B | 1115 | −91.280 | 18.423 | 31.501 | 1.00 | 68.11 | C |
| ATOM | 5194 | CB | THR | B | 1115 | −92.633 | 18.558 | 32.212 | 1.00 | 65.85 | C |
| ATOM | 5195 | OG1 | THR | B | 1115 | −93.305 | 17.292 | 32.202 | 1.00 | 67.56 | O |
| ATOM | 5196 | CG2 | THR | B | 1115 | −93.497 | 19.604 | 31.521 | 1.00 | 73.38 | C |
| ATOM | 5197 | C | THR | B | 1115 | −90.569 | 19.772 | 31.523 | 1.00 | 68.43 | C |
| ATOM | 5198 | O | THR | B | 1115 | −90.415 | 20.427 | 30.507 | 1.00 | 57.91 | O |
| ATOM | 5199 | N | ASN | B | 1116 | −90.150 | 20.180 | 32.704 | 1.00 | 70.30 | N |
| ATOM | 5200 | CA | ASN | B | 1116 | −89.503 | 21.457 | 32.872 | 1.00 | 72.44 | C |
| ATOM | 5201 | CB | ASN | B | 1116 | −89.308 | 21.755 | 34.358 | 1.00 | 75.33 | C |
| ATOM | 5202 | CG | ASN | B | 1116 | −90.613 | 21.695 | 35.133 | 1.00 | 79.72 | C |
| ATOM | 5203 | OD1 | ASN | B | 1116 | −91.620 | 22.280 | 34.726 | 1.00 | 72.22 | O |
| ATOM | 5204 | ND2 | ASN | B | 1116 | −90.607 | 20.973 | 36.247 | 1.00 | 86.79 | N |
| ATOM | 5205 | C | ASN | B | 1116 | −88.165 | 21.546 | 32.105 | 1.00 | 73.11 | C |
| ATOM | 5206 | O | ASN | B | 1116 | −87.850 | 22.547 | 31.484 | 1.00 | 69.76 | O |
| ATOM | 5207 | N | SER | B | 1117 | −87.366 | 20.472 | 32.148 | 1.00 | 71.52 | N |
| ATOM | 5208 | CA | SER | B | 1117 | −86.071 | 20.470 | 31.473 | 1.00 | 61.70 | C |
| ATOM | 5209 | CB | SER | B | 1117 | −85.322 | 19.168 | 31.751 | 1.00 | 57.59 | C |
| ATOM | 5210 | OG | SER | B | 1117 | −85.159 | 18.961 | 33.141 | 1.00 | 55.06 | O |
| ATOM | 5211 | C | SER | B | 1117 | −86.205 | 20.662 | 29.968 | 1.00 | 59.78 | C |
| ATOM | 5212 | O | SER | B | 1117 | −85.519 | 21.446 | 29.346 | 1.00 | 60.48 | O |
| ATOM | 5213 | N | LEU | B | 1118 | −87.164 | 19.976 | 29.403 | 1.00 | 59.30 | N |
| ATOM | 5214 | CA | LEU | B | 1118 | −87.496 | 20.120 | 28.013 | 1.00 | 58.24 | C |
| ATOM | 5215 | CB | LEU | B | 1118 | −88.479 | 19.022 | 27.602 | 1.00 | 53.26 | C |
| ATOM | 5216 | CG | LEU | B | 1118 | −87.890 | 17.610 | 27.562 | 1.00 | 51.13 | C |
| ATOM | 5217 | CD1 | LEU | B | 1118 | −88.980 | 16.565 | 27.419 | 1.00 | 44.12 | C |
| ATOM | 5218 | CD2 | LEU | B | 1118 | −86.878 | 17.481 | 26.434 | 1.00 | 54.90 | C |
| ATOM | 5219 | C | LEU | B | 1118 | −88.066 | 21.506 | 27.709 | 1.00 | 66.44 | C |
| ATOM | 5220 | O | LEU | B | 1118 | −87.780 | 22.095 | 26.682 | 1.00 | 68.42 | O |
| ATOM | 5221 | N | ARG | B | 1119 | −88.882 | 22.033 | 28.620 | 1.00 | 67.85 | N |
| ATOM | 5222 | CA | ARG | B | 1119 | −89.346 | 23.412 | 28.524 | 1.00 | 73.01 | C |
| ATOM | 5223 | CB | ARG | B | 1119 | −90.225 | 23.745 | 29.739 | 1.00 | 76.83 | C |
| ATOM | 5224 | CG | ARG | B | 1119 | −90.637 | 25.205 | 29.868 | 1.00 | 74.17 | C |
| ATOM | 5225 | CD | ARG | B | 1119 | −91.337 | 25.466 | 31.197 | 1.00 | 70.40 | C |
| ATOM | 5226 | NE | ARG | B | 1119 | −91.592 | 26.890 | 31.406 | 1.00 | 85.78 | N |
| ATOM | 5227 | CZ | ARG | B | 1119 | −90.742 | 27.725 | 31.998 | 1.00 | 86.02 | C |
| ATOM | 5228 | NH1 | ARG | B | 1119 | −91.060 | 29.006 | 32.143 | 1.00 | 74.88 | N |
| ATOM | 5229 | NH2 | ARG | B | 1119 | −89.573 | 27.283 | 32.447 | 1.00 | 81.96 | N |
| ATOM | 5230 | C | ARG | B | 1119 | −88.173 | 24.399 | 28.416 | 1.00 | 68.75 | C |
| ATOM | 5231 | O | ARG | B | 1119 | −88.188 | 25.290 | 27.590 | 1.00 | 69.41 | O |
| ATOM | 5232 | N | MET | B | 1120 | −87.142 | 24.198 | 29.242 | 1.00 | 62.85 | N |
| ATOM | 5233 | CA | MET | B | 1120 | −85.953 | 25.051 | 29.260 | 1.00 | 62.54 | C |
| ATOM | 5234 | CB | MET | B | 1120 | −85.186 | 24.862 | 30.564 | 1.00 | 62.35 | C |
| ATOM | 5235 | CG | MET | B | 1120 | −85.982 | 25.263 | 31.785 | 1.00 | 71.41 | C |
| ATOM | 5236 | SD | MET | B | 1120 | −85.020 | 25.114 | 33.297 | 1.00 | 89.24 | S |
| ATOM | 5237 | CE | MET | B | 1120 | −86.184 | 25.776 | 34.487 | 1.00 | 95.26 | C |
| ATOM | 5238 | C | MET | B | 1120 | −85.021 | 24.870 | 28.054 | 1.00 | 62.69 | C |
| ATOM | 5239 | O | MET | B | 1120 | −84.482 | 25.833 | 27.534 | 1.00 | 65.20 | O |
| ATOM | 5240 | N | LEU | B | 1121 | −84.838 | 23.628 | 27.606 | 1.00 | 57.74 | N |
| ATOM | 5241 | CA | LEU | B | 1121 | −84.042 | 23.361 | 26.422 | 1.00 | 56.97 | C |
| ATOM | 5242 | CB | LEU | B | 1121 | −83.934 | 21.854 | 26.172 | 1.00 | 59.43 | C |
| ATOM | 5243 | CG | LEU | B | 1121 | −83.096 | 21.029 | 27.153 | 1.00 | 59.38 | C |
| ATOM | 5244 | CD1 | LEU | B | 1121 | −83.285 | 19.549 | 26.876 | 1.00 | 55.29 | C |
| ATOM | 5245 | CD2 | LEU | B | 1121 | −81.618 | 21.402 | 27.077 | 1.00 | 47.74 | C |
| ATOM | 5246 | C | LEU | B | 1121 | −84.643 | 24.056 | 25.197 | 1.00 | 56.30 | C |
| ATOM | 5247 | O | LEU | B | 1121 | −83.955 | 24.618 | 24.357 | 1.00 | 54.45 | O |
| ATOM | 5248 | N | GLN | B | 1122 | −85.960 | 24.058 | 25.130 | 1.00 | 65.51 | N |
| ATOM | 5249 | CA | GLN | B | 1122 | −86.663 | 24.787 | 24.092 | 1.00 | 65.30 | C |
| ATOM | 5250 | CB | GLN | B | 1122 | −88.147 | 24.466 | 24.209 | 1.00 | 60.73 | C |
| ATOM | 5251 | CG | GLN | B | 1122 | −89.007 | 24.965 | 23.089 | 1.00 | 57.69 | C |
| ATOM | 5252 | CD | GLN | B | 1122 | −90.427 | 24.503 | 23.268 | 1.00 | 68.98 | C |
| ATOM | 5253 | OE1 | GLN | B | 1122 | −90.774 | 23.941 | 24.309 | 1.00 | 71.14 | O |
| ATOM | 5254 | NE2 | GLN | B | 1122 | −91.260 | 24.724 | 22.258 | 1.00 | 74.90 | N |
| ATOM | 5255 | C | GLN | B | 1122 | −86.441 | 26.309 | 24.175 | 1.00 | 62.04 | C |
| ATOM | 5256 | O | GLN | B | 1122 | −86.378 | 26.998 | 23.166 | 1.00 | 59.44 | O |
| ATOM | 5257 | N | GLN | B | 1123 | −86.307 | 26.819 | 25.403 | 1.00 | 62.81 | N |
| ATOM | 5258 | CA | GLN | B | 1123 | −86.087 | 28.242 | 25.629 | 1.00 | 61.24 | C |
| ATOM | 5259 | CB | GLN | B | 1123 | −86.719 | 28.675 | 26.953 | 1.00 | 67.82 | C |
| ATOM | 5260 | CG | GLN | B | 1123 | −88.217 | 28.471 | 27.029 | 1.00 | 69.71 | C |
| ATOM | 5261 | CD | GLN | B | 1123 | −88.779 | 28.861 | 28.378 | 1.00 | 79.10 | C |
| ATOM | 5262 | OE1 | GLN | B | 1123 | −88.044 | 29.302 | 29.265 | 1.00 | 75.37 | O |
| ATOM | 5263 | NE2 | GLN | B | 1123 | −90.088 | 28.700 | 28.544 | 1.00 | 86.84 | N |
| ATOM | 5264 | C | GLN | B | 1123 | −84.601 | 28.620 | 25.646 | 1.00 | 66.40 | C |
| ATOM | 5265 | O | GLN | B | 1123 | −84.252 | 29.762 | 25.918 | 1.00 | 67.67 | O |
| ATOM | 5266 | N | LYS | B | 1124 | −83.739 | 27.647 | 25.329 | 1.00 | 63.56 | N |
| ATOM | 5267 | CA | LYS | B | 1124 | −82.295 | 27.836 | 25.264 | 1.00 | 55.70 | C |
| ATOM | 5268 | CB | LYS | B | 1124 | −81.913 | 28.768 | 24.109 | 1.00 | 52.62 | C |
| ATOM | 5269 | CG | LYS | B | 1124 | −82.511 | 28.331 | 22.780 | 1.00 | 51.99 | C |
| ATOM | 5270 | CD | LYS | B | 1124 | −81.676 | 28.778 | 21.598 | 1.00 | 52.96 | C |
| ATOM | 5271 | CE | LYS | B | 1124 | −82.416 | 28.504 | 20.301 | 1.00 | 71.18 | C |
| ATOM | 5272 | NZ | LYS | B | 1124 | −82.925 | 27.104 | 20.237 | 1.00 | 60.81 | N |
| ATOM | 5273 | C | LYS | B | 1124 | −81.648 | 28.255 | 26.580 | 1.00 | 57.10 | C |
| ATOM | 5274 | O | LYS | B | 1124 | −80.713 | 29.036 | 26.589 | 1.00 | 60.60 | O |
| ATOM | 5275 | N | ARG | B | 1125 | −82.182 | 27.719 | 27.686 | 1.00 | 58.59 | N |
| ATOM | 5276 | CA | ARG | B | 1125 | −81.675 | 27.978 | 29.040 | 1.00 | 65.00 | C |
| ATOM | 5277 | CB | ARG | B | 1125 | −82.827 | 28.257 | 30.011 | 1.00 | 66.12 | C |
| ATOM | 5278 | CG | ARG | B | 1125 | −83.504 | 29.598 | 29.764 | 1.00 | 65.07 | C |
| ATOM | 5279 | CD | ARG | B | 1125 | −84.876 | 29.680 | 30.411 | 1.00 | 76.31 | C |
| ATOM | 5280 | NE | ARG | B | 1125 | −84.817 | 29.580 | 31.868 | 1.00 | 87.91 | N |
| ATOM | 5281 | CZ | ARG | B | 1125 | −85.886 | 29.602 | 32.661 | 1.00 | 92.55 | C |
| ATOM | 5282 | NH1 | ARG | B | 1125 | −85.747 | 29.503 | 33.977 | 1.00 | 91.83 | N |
| ATOM | 5283 | NH2 | ARG | B | 1125 | −87.099 | 29.721 | 32.136 | 1.00 | 82.96 | N |
| ATOM | 5284 | C | ARG | B | 1125 | −80.791 | 26.840 | 29.537 | 1.00 | 61.92 | C |
| ATOM | 5285 | O | ARG | B | 1125 | −81.131 | 26.126 | 30.462 | 1.00 | 61.92 | O |
| ATOM | 5286 | N | TRP | B | 1126 | −79.689 | 26.620 | 28.824 | 1.00 | 63.70 | N |
| ATOM | 5287 | CA | TRP | B | 1126 | −78.885 | 25.407 | 28.947 | 1.00 | 58.61 | C |
| ATOM | 5288 | CB | TRP | B | 1126 | −77.689 | 25.452 | 27.990 | 1.00 | 44.14 | C |
| ATOM | 5289 | CG | TRP | B | 1126 | −78.016 | 25.934 | 26.612 | 1.00 | 49.25 | C |
| ATOM | 5290 | CD1 | TRP | B | 1126 | −77.521 | 27.049 | 26.001 | 1.00 | 55.10 | C |
| ATOM | 5291 | NE1 | TRP | B | 1126 | −78.048 | 27.173 | 24.741 | 1.00 | 50.60 | N |
| ATOM | 5292 | CE2 | TRP | B | 1126 | −78.905 | 26.131 | 24.513 | 1.00 | 43.19 | C |
| ATOM | 5293 | CD2 | TRP | B | 1126 | −78.913 | 25.327 | 25.672 | 1.00 | 45.08 | C |
| ATOM | 5294 | CE3 | TRP | B | 1126 | −79.719 | 24.186 | 25.694 | 1.00 | 45.48 | C |
| ATOM | 5295 | CZ3 | TRP | B | 1126 | −80.482 | 23.890 | 24.574 | 1.00 | 48.74 | C |
| ATOM | 5296 | CH2 | TRP | B | 1126 | −80.451 | 24.709 | 23.438 | 1.00 | 52.42 | C |
| ATOM | 5297 | CZ2 | TRP | B | 1126 | −79.671 | 25.830 | 23.389 | 1.00 | 45.23 | C |
| ATOM | 5298 | C | TRP | B | 1126 | −78.357 | 25.157 | 30.357 | 1.00 | 59.08 | C |
| ATOM | 5299 | O | TRP | B | 1126 | −78.357 | 24.039 | 30.857 | 1.00 | 56.16 | O |
| ATOM | 5300 | N | ASP | B | 1127 | −77.915 | 26.234 | 30.987 | 1.00 | 66.05 | N |
| ATOM | 5301 | CA | ASP | B | 1127 | −77.284 | 26.174 | 32.291 | 1.00 | 74.08 | C |
| ATOM | 5302 | CB | ASP | B | 1127 | −76.737 | 27.543 | 32.701 | 1.00 | 90.20 | C |
| ATOM | 5303 | CG | ASP | B | 1127 | −75.488 | 27.925 | 31.934 | 1.00 | 103.11 | C |
| ATOM | 5304 | OD1 | ASP | B | 1127 | −74.809 | 27.019 | 31.403 | 1.00 | 96.44 | O |
| ATOM | 5305 | OD2 | ASP | B | 1127 | −75.182 | 29.134 | 31.871 | 1.00 | 120.63 | O |
| ATOM | 5306 | C | ASP | B | 1127 | −78.231 | 25.649 | 33.372 | 1.00 | 69.91 | C |
| ATOM | 5307 | O | ASP | B | 1127 | −77.816 | 24.908 | 34.242 | 1.00 | 67.35 | O |
| ATOM | 5308 | N | GLU | B | 1128 | −79.511 | 26.045 | 33.286 | 1.00 | 74.79 | N |
| ATOM | 5309 | CA | GLU | B | 1128 | −80.552 | 25.601 | 34.227 | 1.00 | 73.00 | C |
| ATOM | 5310 | CB | GLU | B | 1128 | −81.770 | 26.524 | 34.165 | 1.00 | 73.67 | C |
| ATOM | 5311 | CG | GLU | B | 1128 | −81.532 | 27.942 | 34.631 | 1.00 | 79.50 | C |
| ATOM | 5312 | CD | GLU | B | 1128 | −82.815 | 28.745 | 34.667 | 1.00 | 77.32 | C |
| ATOM | 5313 | OE1 | GLU | B | 1128 | −82.827 | 29.875 | 34.135 | 1.00 | 79.20 | O |
| ATOM | 5314 | OE2 | GLU | B | 1128 | −83.812 | 28.242 | 35.225 | 1.00 | 72.51 | O |
| ATOM | 5315 | C | GLU | B | 1128 | −81.029 | 24.166 | 33.962 | 1.00 | 66.73 | C |
| ATOM | 5316 | O | GLU | B | 1128 | −81.267 | 23.401 | 34.893 | 1.00 | 65.56 | O |
| ATOM | 5317 | N | ALA | B | 1129 | −81.220 | 23.839 | 32.665 | 1.00 | 61.17 | N |
| ATOM | 5318 | CA | ALA | B | 1129 | −81.707 | 22.527 | 32.242 | 1.00 | 56.60 | C |
| ATOM | 5319 | CB | ALA | B | 1129 | −81.933 | 22.505 | 30.734 | 1.00 | 49.42 | C |
| ATOM | 5320 | C | ALA | B | 1129 | −80.755 | 21.417 | 32.660 | 1.00 | 56.59 | C |
| ATOM | 5321 | O | ALA | B | 1129 | −81.158 | 20.356 | 33.095 | 1.00 | 52.05 | O |
| ATOM | 5322 | N | ALA | B | 1130 | −79.467 | 21.725 | 32.593 | 1.00 | 59.40 | N |
| ATOM | 5323 | CA | ALA | B | 1130 | −78.411 | 20.847 | 33.046 | 1.00 | 49.55 | C |
| ATOM | 5324 | CB | ALA | B | 1130 | −77.053 | 21.495 | 32.828 | 1.00 | 46.87 | C |
| ATOM | 5325 | C | ALA | B | 1130 | −78.592 | 20.440 | 34.521 | 1.00 | 55.15 | C |
| ATOM | 5326 | O | ALA | B | 1130 | −78.468 | 19.281 | 34.874 | 1.00 | 54.93 | O |
| ATOM | 5327 | N | VAL | B | 1131 | −78.859 | 21.419 | 35.388 | 1.00 | 59.80 | N |
| ATOM | 5328 | CA | VAL | B | 1131 | −79.158 | 21.147 | 36.797 | 1.00 | 65.84 | C |
| ATOM | 5329 | CB | VAL | B | 1131 | −79.357 | 22.465 | 37.580 | 1.00 | 60.37 | C |
| ATOM | 5330 | CG1 | VAL | B | 1131 | −79.786 | 22.178 | 39.014 | 1.00 | 59.18 | C |
| ATOM | 5331 | CG2 | VAL | B | 1131 | −78.084 | 23.305 | 37.546 | 1.00 | 53.37 | C |
| ATOM | 5332 | C | VAL | B | 1131 | −80.402 | 20.256 | 36.992 | 1.00 | 65.16 | C |
| ATOM | 5333 | O | VAL | B | 1131 | −80.389 | 19.293 | 37.752 | 1.00 | 62.57 | O |
| ATOM | 5334 | N | ASN | B | 1132 | −81.479 | 20.588 | 36.265 | 1.00 | 60.29 | N |
| ATOM | 5335 | CA | ASN | B | 1132 | −82.735 | 19.822 | 36.312 | 1.00 | 58.67 | C |
| ATOM | 5336 | CB | ASN | B | 1132 | −83.805 | 20.477 | 35.446 | 1.00 | 62.66 | C |
| ATOM | 5337 | CG | ASN | B | 1132 | −84.301 | 21.776 | 36.030 | 1.00 | 69.70 | C |
| ATOM | 5338 | OD1 | ASN | B | 1132 | −84.043 | 22.082 | 37.195 | 1.00 | 67.73 | O |
| ATOM | 5339 | ND2 | ASN | B | 1132 | −85.026 | 22.548 | 35.228 | 1.00 | 74.38 | N |
| ATOM | 5340 | C | ASN | B | 1132 | −82.560 | 18.359 | 35.927 | 1.00 | 57.23 | C |
| ATOM | 5341 | O | ASN | B | 1132 | −83.150 | 17.472 | 36.504 | 1.00 | 62.93 | O |
| ATOM | 5342 | N | LEU | B | 1133 | −81.719 | 18.123 | 34.945 | 1.00 | 56.98 | N |
| ATOM | 5343 | CA | LEU | B | 1133 | −81.406 | 16.789 | 34.510 | 1.00 | 51.84 | C |
| ATOM | 5344 | CB | LEU | B | 1133 | −80.671 | 16.822 | 33.169 | 1.00 | 43.04 | C |
| ATOM | 5345 | CG | LEU | B | 1133 | −81.566 | 17.285 | 32.015 | 1.00 | 38.84 | C |
| ATOM | 5346 | CD1 | LEU | B | 1133 | −80.742 | 17.630 | 30.794 | 1.00 | 47.74 | C |
| ATOM | 5347 | CD2 | LEU | B | 1133 | −82.599 | 16.220 | 31.671 | 1.00 | 36.61 | C |
| ATOM | 5348 | C | LEU | B | 1133 | −80.625 | 16.030 | 35.578 | 1.00 | 47.29 | C |
| ATOM | 5349 | O | LEU | B | 1133 | −80.891 | 14.879 | 35.832 | 1.00 | 45.21 | O |
| ATOM | 5350 | N | ALA | B | 1134 | −79.677 | 16.696 | 36.215 | 1.00 | 49.40 | N |
| ATOM | 5351 | CA | ALA | B | 1134 | −78.895 | 16.115 | 37.313 | 1.00 | 50.66 | C |
| ATOM | 5352 | CB | ALA | B | 1134 | −77.900 | 17.139 | 37.846 | 1.00 | 55.92 | C |
| ATOM | 5353 | C | ALA | B | 1134 | −79.740 | 15.537 | 38.459 | 1.00 | 56.89 | C |
| ATOM | 5354 | O | ALA | B | 1134 | −79.343 | 14.602 | 39.139 | 1.00 | 54.76 | O |
| ATOM | 5355 | N | LYS | B | 1135 | −80.883 | 16.159 | 38.699 | 1.00 | 57.92 | N |
| ATOM | 5356 | CA | LYS | B | 1135 | −81.787 | 15.780 | 39.774 | 1.00 | 53.31 | C |
| ATOM | 5357 | CB | LYS | B | 1135 | −82.819 | 16.882 | 40.027 | 1.00 | 52.40 | C |
| ATOM | 5358 | CG | LYS | B | 1135 | −82.205 | 18.125 | 40.661 | 1.00 | 56.28 | C |
| ATOM | 5359 | CD | LYS | B | 1135 | −83.155 | 19.312 | 40.657 | 1.00 | 66.00 | C |
| ATOM | 5360 | CE | LYS | B | 1135 | −82.435 | 20.582 | 41.100 | 1.00 | 60.68 | C |
| ATOM | 5361 | NZ | LYS | B | 1135 | −83.253 | 21.813 | 40.896 | 1.00 | 56.07 | N |
| ATOM | 5362 | C | LYS | B | 1135 | −82.461 | 14.372 | 39.655 | 1.00 | 53.13 | C |
| ATOM | 5363 | O | LYS | B | 1135 | −83.037 | 13.870 | 40.605 | 1.00 | 61.90 | O |
| ATOM | 5364 | N | SER | B | 1136 | −82.421 | 13.768 | 38.486 | 1.00 | 52.54 | N |
| ATOM | 5365 | CA | SER | B | 1136 | −82.949 | 12.415 | 38.216 | 1.00 | 54.05 | C |
| ATOM | 5366 | CB | SER | B | 1136 | −83.000 | 12.176 | 36.708 | 1.00 | 58.40 | C |
| ATOM | 5367 | OG | SER | B | 1136 | −81.695 | 12.230 | 36.153 | 1.00 | 54.66 | O |
| ATOM | 5368 | C | SER | B | 1136 | −82.247 | 11.193 | 38.879 | 1.00 | 51.22 | C |
| ATOM | 5369 | O | SER | B | 1136 | −81.037 | 11.144 | 38.987 | 1.00 | 48.94 | O |
| ATOM | 5370 | N | ARG | B | 1137 | −83.057 | 10.118 | 39.095 | 1.00 | 51.42 | N |
| ATOM | 5371 | CA | ARG | B | 1137 | −82.572 | 8.729 | 39.343 | 1.00 | 42.34 | C |
| ATOM | 5372 | CB | ARG | B | 1137 | −83.741 | 7.758 | 39.552 | 1.00 | 41.94 | C |
| ATOM | 5373 | CG | ARG | B | 1137 | −84.624 | 8.082 | 40.748 | 1.00 | 48.07 | C |
| ATOM | 5374 | CD | ARG | B | 1137 | −85.715 | 7.032 | 40.969 | 1.00 | 47.92 | C |
| ATOM | 5375 | NE | ARG | B | 1137 | −86.937 | 7.619 | 41.525 | 1.00 | 44.76 | N |
| ATOM | 5376 | CZ | ARG | B | 1137 | −87.153 | 7.816 | 42.824 | 1.00 | 53.45 | C |
| ATOM | 5377 | NH1 | ARG | B | 1137 | −86.228 | 7.480 | 43.710 | 1.00 | 65.82 | N |
| ATOM | 5378 | NH2 | ARG | B | 1137 | −88.288 | 8.358 | 43.245 | 1.00 | 47.78 | N |
| ATOM | 5379 | C | ARG | B | 1137 | −81.659 | 8.196 | 38.233 | 1.00 | 46.69 | C |
| ATOM | 5380 | O | ARG | B | 1137 | −80.787 | 7.386 | 38.467 | 1.00 | 42.89 | O |
| ATOM | 5381 | N | TRP | B | 1138 | −81.867 | 8.689 | 37.013 | 1.00 | 51.66 | N |
| ATOM | 5382 | CA | TRP | B | 1138 | −80.977 | 8.447 | 35.877 | 1.00 | 47.02 | C |
| ATOM | 5383 | CB | TRP | B | 1138 | −81.462 | 9.294 | 34.692 | 1.00 | 47.48 | C |
| ATOM | 5384 | CG | TRP | B | 1138 | −80.568 | 9.351 | 33.498 | 1.00 | 37.25 | C |
| ATOM | 5385 | CD1 | TRP | B | 1138 | −80.026 | 8.297 | 32.832 | 1.00 | 42.88 | C |
| ATOM | 5386 | NE1 | TRP | B | 1138 | −79.278 | 8.741 | 31.771 | 1.00 | 47.97 | N |
| ATOM | 5387 | CE2 | TRP | B | 1138 | −79.340 | 10.107 | 31.724 | 1.00 | 45.00 | C |
| ATOM | 5388 | CD2 | TRP | B | 1138 | −80.155 | 10.528 | 32.794 | 1.00 | 38.02 | C |
| ATOM | 5389 | CE3 | TRP | B | 1138 | −80.379 | 11.895 | 32.970 | 1.00 | 41.89 | C |
| ATOM | 5390 | CZ3 | TRP | B | 1138 | −79.790 | 12.785 | 32.084 | 1.00 | 41.88 | C |
| ATOM | 5391 | CH2 | TRP | B | 1138 | −78.988 | 12.334 | 31.029 | 1.00 | 34.59 | C |
| ATOM | 5392 | CZ2 | TRP | B | 1138 | −78.751 | 11.003 | 30.833 | 1.00 | 39.12 | C |
| ATOM | 5393 | C | TRP | B | 1138 | −79.543 | 8.786 | 36.231 | 1.00 | 41.79 | C |
| ATOM | 5394 | O | TRP | B | 1138 | −78.650 | 7.995 | 36.058 | 1.00 | 46.37 | O |
| ATOM | 5395 | N | TYR | B | 1139 | −79.358 | 9.995 | 36.734 | 1.00 | 42.44 | N |
| ATOM | 5396 | CA | TYR | B | 1139 | −78.070 | 10.495 | 37.136 | 1.00 | 46.70 | C |
| ATOM | 5397 | CB | TYR | B | 1139 | −78.181 | 11.968 | 37.532 | 1.00 | 51.91 | C |
| ATOM | 5398 | CG | TYR | B | 1139 | −76.867 | 12.699 | 37.724 | 1.00 | 54.55 | C |
| ATOM | 5399 | CD2 | TYR | B | 1139 | −76.376 | 13.550 | 36.738 | 1.00 | 52.59 | C |
| ATOM | 5400 | CE2 | TYR | B | 1139 | −75.197 | 14.246 | 36.918 | 1.00 | 54.23 | C |
| ATOM | 5401 | CZ | TYR | B | 1139 | −74.501 | 14.101 | 38.100 | 1.00 | 54.43 | C |
| ATOM | 5402 | OH | TYR | B | 1139 | −73.327 | 14.791 | 38.295 | 1.00 | 54.61 | O |
| ATOM | 5403 | CE1 | TYR | B | 1139 | −74.975 | 13.272 | 39.094 | 1.00 | 49.55 | C |
| ATOM | 5404 | CD1 | TYR | B | 1139 | −76.149 | 12.586 | 38.908 | 1.00 | 47.34 | C |
| ATOM | 5405 | C | TYR | B | 1139 | −77.478 | 9.704 | 38.288 | 1.00 | 47.16 | C |
| ATOM | 5406 | O | TYR | B | 1139 | −76.305 | 9.419 | 38.302 | 1.00 | 52.73 | O |
| ATOM | 5407 | N | ASN | B | 1140 | −78.290 | 9.315 | 39.249 | 1.00 | 44.15 | N |
| ATOM | 5408 | CA | ASN | B | 1140 | −77.761 | 8.563 | 40.372 | 1.00 | 43.57 | C |
| ATOM | 5409 | CB | ASN | B | 1140 | −78.823 | 8.399 | 41.460 | 1.00 | 44.40 | C |
| ATOM | 5410 | CG | ASN | B | 1140 | −79.225 | 9.721 | 42.075 | 1.00 | 53.01 | C |
| ATOM | 5411 | OD1 | ASN | B | 1140 | −80.398 | 10.093 | 42.071 | 1.00 | 60.52 | O |
| ATOM | 5412 | ND2 | ASN | B | 1140 | −78.245 | 10.443 | 42.611 | 1.00 | 57.07 | N |
| ATOM | 5413 | C | ASN | B | 1140 | −77.216 | 7.190 | 39.967 | 1.00 | 48.82 | C |
| ATOM | 5414 | O | ASN | B | 1140 | −76.210 | 6.748 | 40.486 | 1.00 | 61.63 | O |
| ATOM | 5415 | N | GLN | B | 1141 | −77.959 | 6.480 | 39.115 | 1.00 | 50.69 | N |
| ATOM | 5416 | CA | GLN | B | 1141 | −77.616 | 5.109 | 38.742 | 1.00 | 52.18 | C |
| ATOM | 5417 | CB | GLN | B | 1141 | −78.829 | 4.353 | 38.209 | 1.00 | 46.60 | C |
| ATOM | 5418 | CG | GLN | B | 1141 | −78.547 | 2.875 | 38.041 | 1.00 | 47.82 | C |
| ATOM | 5419 | CD | GLN | B | 1141 | −79.705 | 2.133 | 37.438 | 1.00 | 61.73 | C |
| ATOM | 5420 | OE1 | GLN | B | 1141 | −80.779 | 2.699 | 37.245 | 1.00 | 71.09 | O |
| ATOM | 5421 | NE2 | GLN | B | 1141 | −79.499 | 0.854 | 37.131 | 1.00 | 69.74 | N |
| ATOM | 5422 | C | GLN | B | 1141 | −76.443 | 4.994 | 37.783 | 1.00 | 52.90 | C |
| ATOM | 5423 | O | GLN | B | 1141 | −75.603 | 4.111 | 37.907 | 1.00 | 55.70 | O |
| ATOM | 5424 | N | THR | B | 1142 | −76.387 | 5.915 | 36.828 | 1.00 | 49.59 | N |
| ATOM | 5425 | CA | THR | B | 1142 | −75.306 | 5.936 | 35.860 | 1.00 | 56.77 | C |
| ATOM | 5426 | CB | THR | B | 1142 | −75.772 | 5.401 | 34.479 | 1.00 | 51.99 | C |
| ATOM | 5427 | OG1 | THR | B | 1142 | −76.888 | 6.168 | 34.007 | 1.00 | 43.42 | O |
| ATOM | 5428 | CG2 | THR | B | 1142 | −76.186 | 3.938 | 34.576 | 1.00 | 54.75 | C |
| ATOM | 5429 | C | THR | B | 1142 | −74.782 | 7.362 | 35.705 | 1.00 | 52.54 | C |
| ATOM | 5430 | O | THR | B | 1142 | −75.030 | 7.996 | 34.703 | 1.00 | 45.66 | O |
| ATOM | 5431 | N | PRO | B | 1143 | −74.066 | 7.939 | 36.680 | 1.00 | 55.19 | N |
| ATOM | 5432 | CA | PRO | B | 1143 | −73.661 | 9.329 | 36.611 | 1.00 | 53.13 | C |
| ATOM | 5433 | CB | PRO | B | 1143 | −72.962 | 9.558 | 37.964 | 1.00 | 51.44 | C |
| ATOM | 5434 | CG | PRO | B | 1143 | −73.291 | 8.335 | 38.807 | 1.00 | 47.50 | C |
| ATOM | 5435 | CD | PRO | B | 1143 | −73.440 | 7.238 | 37.822 | 1.00 | 47.45 | C |
| ATOM | 5436 | C | PRO | B | 1143 | −72.703 | 9.613 | 35.472 | 1.00 | 51.52 | C |
| ATOM | 5437 | O | PRO | B | 1143 | −72.625 | 10.722 | 34.999 | 1.00 | 48.28 | O |
| ATOM | 5438 | N | ASN | B | 1144 | −71.925 | 8.634 | 35.087 | 1.00 | 55.13 | N |
| ATOM | 5439 | CA | ASN | B | 1144 | −70.926 | 8.839 | 34.085 | 1.00 | 52.28 | C |
| ATOM | 5440 | CB | ASN | B | 1144 | −69.947 | 7.712 | 34.152 | 1.00 | 57.59 | C |
| ATOM | 5441 | CG | ASN | B | 1144 | −68.990 | 7.853 | 35.316 | 1.00 | 57.82 | C |
| ATOM | 5442 | OD1 | ASN | B | 1144 | −68.617 | 8.969 | 35.691 | 1.00 | 54.68 | O |
| ATOM | 5443 | ND2 | ASN | B | 1144 | −68.585 | 6.731 | 35.892 | 1.00 | 71.45 | N |
| ATOM | 5444 | C | ASN | B | 1144 | −71.494 | 9.123 | 32.707 | 1.00 | 50.30 | C |
| ATOM | 5445 | O | ASN | B | 1144 | −71.055 | 10.035 | 32.057 | 1.00 | 46.98 | O |
| ATOM | 5446 | N | ARG | B | 1145 | −72.495 | 8.357 | 32.278 | 1.00 | 47.12 | N |
| ATOM | 5447 | CA | ARG | B | 1145 | −73.157 | 8.579 | 30.987 | 1.00 | 38.59 | C |
| ATOM | 5448 | CB | ARG | B | 1145 | −73.951 | 7.342 | 30.554 | 1.00 | 37.03 | C |
| ATOM | 5449 | CG | ARG | B | 1145 | −74.654 | 7.479 | 29.204 | 1.00 | 33.90 | C |
| ATOM | 5450 | CD | ARG | B | 1145 | −75.364 | 6.185 | 28.813 | 1.00 | 37.71 | C |
| ATOM | 5451 | NE | ARG | B | 1145 | −76.102 | 6.306 | 27.555 | 1.00 | 37.06 | N |
| ATOM | 5452 | CZ | ARG | B | 1145 | −76.766 | 5.308 | 26.976 | 1.00 | 36.99 | C |
| ATOM | 5453 | NH1 | ARG | B | 1145 | −76.789 | 4.107 | 27.536 | 1.00 | 39.80 | N |
| ATOM | 5454 | NH2 | ARG | B | 1145 | −77.406 | 5.507 | 25.832 | 1.00 | 36.17 | N |
| ATOM | 5455 | C | ARG | B | 1145 | −74.055 | 9.792 | 31.031 | 1.00 | 38.25 | C |
| ATOM | 5456 | O | ARG | B | 1145 | −74.156 | 10.536 | 30.084 | 1.00 | 36.01 | O |
| ATOM | 5457 | N | ALA | B | 1146 | −74.665 | 10.005 | 32.184 | 1.00 | 40.47 | N |
| ATOM | 5458 | CA | ALA | B | 1146 | −75.478 | 11.166 | 32.430 | 1.00 | 41.48 | C |
| ATOM | 5459 | CB | ALA | B | 1146 | −76.148 | 11.054 | 33.794 | 1.00 | 44.85 | C |
| ATOM | 5460 | C | ALA | B | 1146 | −74.718 | 12.469 | 32.325 | 1.00 | 46.96 | C |
| ATOM | 5461 | O | ALA | B | 1146 | −75.163 | 13.387 | 31.695 | 1.00 | 46.81 | O |
| ATOM | 5462 | N | LYS | B | 1147 | −73.545 | 12.546 | 32.927 | 1.00 | 46.35 | N |
| ATOM | 5463 | CA | LYS | B | 1147 | −72.726 | 13.743 | 32.866 | 1.00 | 49.85 | C |
| ATOM | 5464 | CB | LYS | B | 1147 | −71.467 | 13.588 | 33.727 | 1.00 | 58.65 | C |
| ATOM | 5465 | CG | LYS | B | 1147 | −71.684 | 13.740 | 35.226 | 1.00 | 49.26 | C |
| ATOM | 5466 | CD | LYS | B | 1147 | −70.373 | 13.534 | 35.972 | 1.00 | 51.07 | C |
| ATOM | 5467 | CE | LYS | B | 1147 | −70.586 | 13.446 | 37.473 | 1.00 | 48.53 | C |
| ATOM | 5468 | NZ | LYS | B | 1147 | −69.331 | 13.087 | 38.186 | 1.00 | 55.52 | N |
| ATOM | 5469 | C | LYS | B | 1147 | −72.329 | 14.105 | 31.434 | 1.00 | 46.17 | C |
| ATOM | 5470 | O | LYS | B | 1147 | −72.460 | 15.237 | 31.025 | 1.00 | 44.54 | O |
| ATOM | 5471 | N | ARG | B | 1148 | −71.858 | 13.122 | 30.668 | 1.00 | 39.63 | N |
| ATOM | 5472 | CA | ARG | B | 1148 | −71.467 | 13.352 | 29.289 | 1.00 | 43.21 | C |
| ATOM | 5473 | CB | ARG | B | 1148 | −70.886 | 12.077 | 28.668 | 1.00 | 39.68 | C |
| ATOM | 5474 | CG | ARG | B | 1148 | −69.619 | 11.586 | 29.340 | 1.00 | 37.25 | C |
| ATOM | 5475 | CD | ARG | B | 1148 | −68.940 | 10.507 | 28.522 | 1.00 | 39.54 | C |
| ATOM | 5476 | NE | ARG | B | 1148 | −69.640 | 9.233 | 28.611 | 1.00 | 41.70 | N |
| ATOM | 5477 | CZ | ARG | B | 1148 | −69.354 | 8.286 | 29.497 | 1.00 | 46.80 | C |
| ATOM | 5478 | NH1 | ARG | B | 1148 | −68.378 | 8.468 | 30.377 | 1.00 | 55.18 | N |
| ATOM | 5479 | NH2 | ARG | B | 1148 | −70.042 | 7.155 | 29.503 | 1.00 | 47.38 | N |
| ATOM | 5480 | C | ARG | B | 1148 | −72.633 | 13.854 | 28.450 | 1.00 | 43.74 | C |
| ATOM | 5481 | O | ARG | B | 1148 | −72.522 | 14.822 | 27.736 | 1.00 | 39.28 | O |
| ATOM | 5482 | N | VAL | B | 1149 | −73.776 | 13.196 | 28.596 | 1.00 | 45.41 | N |
| ATOM | 5483 | CA | VAL | B | 1149 | −74.985 | 13.541 | 27.870 | 1.00 | 41.53 | C |
| ATOM | 5484 | CB | VAL | B | 1149 | −76.097 | 12.472 | 28.051 | 1.00 | 40.03 | C |
| ATOM | 5485 | CG1 | VAL | B | 1149 | −77.437 | 12.974 | 27.528 | 1.00 | 37.03 | C |
| ATOM | 5486 | CG2 | VAL | B | 1149 | −75.711 | 11.193 | 27.338 | 1.00 | 38.21 | C |
| ATOM | 5487 | C | VAL | B | 1149 | −75.503 | 14.938 | 28.243 | 1.00 | 41.19 | C |
| ATOM | 5488 | O | VAL | B | 1149 | −75.780 | 15.754 | 27.390 | 1.00 | 41.52 | O |
| ATOM | 5489 | N | ILE | B | 1150 | −75.591 | 15.219 | 29.531 | 1.00 | 39.37 | N |
| ATOM | 5490 | CA | ILE | B | 1150 | −76.011 | 16.516 | 30.021 | 1.00 | 41.42 | C |
| ATOM | 5491 | CB | ILE | B | 1150 | −76.200 | 16.502 | 31.548 | 1.00 | 39.81 | C |
| ATOM | 5492 | CG1 | ILE | B | 1150 | −77.364 | 15.580 | 31.913 | 1.00 | 42.31 | C |
| ATOM | 5493 | CD1 | ILE | B | 1150 | −77.454 | 15.261 | 33.387 | 1.00 | 50.65 | C |
| ATOM | 5494 | CG2 | ILE | B | 1150 | −76.470 | 17.902 | 32.076 | 1.00 | 37.92 | C |
| ATOM | 5495 | C | ILE | B | 1150 | −75.049 | 17.616 | 29.608 | 1.00 | 43.73 | C |
| ATOM | 5496 | O | ILE | B | 1150 | −75.446 | 18.704 | 29.239 | 1.00 | 41.17 | O |
| ATOM | 5497 | N | ALA | B | 1151 | −73.759 | 17.315 | 29.684 | 1.00 | 45.52 | N |
| ATOM | 5498 | CA | ALA | B | 1151 | −72.712 | 18.233 | 29.280 | 1.00 | 42.84 | C |
| ATOM | 5499 | CB | ALA | B | 1151 | −71.331 | 17.633 | 29.541 | 1.00 | 40.57 | C |
| ATOM | 5500 | C | ALA | B | 1151 | −72.851 | 18.618 | 27.829 | 1.00 | 41.65 | C |
| ATOM | 5501 | O | ALA | B | 1151 | −72.731 | 19.761 | 27.488 | 1.00 | 42.92 | O |
| ATOM | 5502 | N | THR | B | 1152 | −73.169 | 17.645 | 26.988 | 1.00 | 39.26 | N |
| ATOM | 5503 | CA | THR | B | 1152 | −73.449 | 17.866 | 25.582 | 1.00 | 34.36 | C |
| ATOM | 5504 | CB | THR | B | 1152 | −73.744 | 16.546 | 24.849 | 1.00 | 33.62 | C |
| ATOM | 5505 | OG1 | THR | B | 1152 | −72.759 | 15.568 | 25.207 | 1.00 | 35.66 | O |
| ATOM | 5506 | CG2 | THR | B | 1152 | −73.720 | 16.757 | 23.345 | 1.00 | 32.92 | C |
| ATOM | 5507 | C | THR | B | 1152 | −74.604 | 18.847 | 25.387 | 1.00 | 43.47 | C |
| ATOM | 5508 | O | THR | B | 1152 | −74.494 | 19.780 | 24.620 | 1.00 | 48.47 | O |
| ATOM | 5509 | N | PHE | B | 1153 | −75.693 | 18.664 | 26.153 | 1.00 | 48.92 | N |
| ATOM | 5510 | CA | PHE | B | 1153 | −76.787 | 19.642 | 26.189 | 1.00 | 48.43 | C |
| ATOM | 5511 | CB | PHE | B | 1153 | −77.857 | 19.217 | 27.199 | 1.00 | 41.44 | C |
| ATOM | 5512 | CG | PHE | B | 1153 | −78.847 | 18.218 | 26.680 | 1.00 | 36.22 | C |
| ATOM | 5513 | CD1 | PHE | B | 1153 | −79.737 | 18.560 | 25.679 | 1.00 | 40.41 | C |
| ATOM | 5514 | CE1 | PHE | B | 1153 | −80.674 | 17.645 | 25.215 | 1.00 | 42.31 | C |
| ATOM | 5515 | CZ | PHE | B | 1153 | −80.740 | 16.381 | 25.767 | 1.00 | 37.41 | C |
| ATOM | 5516 | CE2 | PHE | B | 1153 | −79.866 | 16.031 | 26.781 | 1.00 | 40.60 | C |
| ATOM | 5517 | CD2 | PHE | B | 1153 | −78.929 | 16.953 | 27.239 | 1.00 | 40.96 | C |
| ATOM | 5518 | C | PHE | B | 1153 | −76.304 | 21.056 | 26.608 | 1.00 | 46.28 | C |
| ATOM | 5519 | O | PHE | B | 1153 | −76.618 | 22.043 | 25.984 | 1.00 | 42.18 | O |
| ATOM | 5520 | N | ARG | B | 1154 | −75.540 | 21.161 | 27.682 | 1.00 | 38.91 | N |
| ATOM | 5521 | CA | ARG | B | 1154 | −75.065 | 22.472 | 28.106 | 1.00 | 42.91 | C |
| ATOM | 5522 | CB | ARG | B | 1154 | −74.280 | 22.376 | 29.416 | 1.00 | 43.31 | C |
| ATOM | 5523 | CG | ARG | B | 1154 | −73.778 | 23.722 | 29.906 | 1.00 | 50.45 | C |
| ATOM | 5524 | CD | ARG | B | 1154 | −73.082 | 23.620 | 31.245 | 1.00 | 69.91 | C |
| ATOM | 5525 | NE | ARG | B | 1154 | −72.628 | 24.933 | 31.698 | 1.00 | 110.21 | N |
| ATOM | 5526 | CZ | ARG | B | 1154 | −72.026 | 25.162 | 32.862 | 1.00 | 123.12 | C |
| ATOM | 5527 | NH1 | ARG | B | 1154 | −71.799 | 24.163 | 33.705 | 1.00 | 118.95 | N |
| ATOM | 5528 | NH2 | ARG | B | 1154 | −71.650 | 26.395 | 33.184 | 1.00 | 125.60 | N |
| ATOM | 5529 | C | ARG | B | 1154 | −74.223 | 23.225 | 27.054 | 1.00 | 46.82 | C |
| ATOM | 5530 | O | ARG | B | 1154 | −74.437 | 24.393 | 26.764 | 1.00 | 49.76 | O |
| ATOM | 5531 | N | THR | B | 1155 | −73.178 | 22.551 | 26.586 | 1.00 | 44.14 | N |
| ATOM | 5532 | CA | THR | B | 1155 | −72.170 | 23.170 | 25.774 | 1.00 | 41.05 | C |
| ATOM | 5533 | CB | THR | B | 1155 | −70.796 | 22.485 | 25.965 | 1.00 | 49.02 | C |
| ATOM | 5534 | OG1 | THR | B | 1155 | −70.867 | 21.113 | 25.551 | 1.00 | 46.02 | O |
| ATOM | 5535 | CG2 | THR | B | 1155 | −70.375 | 22.548 | 27.427 | 1.00 | 52.47 | C |
| ATOM | 5536 | C | THR | B | 1155 | −72.521 | 23.192 | 24.313 | 1.00 | 44.90 | C |
| ATOM | 5537 | O | THR | B | 1155 | −72.146 | 24.101 | 23.598 | 1.00 | 53.77 | O |
| ATOM | 5538 | N | GLY | B | 1156 | −73.235 | 22.173 | 23.866 | 1.00 | 44.76 | N |
| ATOM | 5539 | CA | GLY | B | 1156 | −73.575 | 22.014 | 22.485 | 1.00 | 43.17 | C |
| ATOM | 5540 | C | GLY | B | 1156 | −72.435 | 21.629 | 21.649 | 1.00 | 46.23 | C |
| ATOM | 5541 | O | GLY | B | 1156 | −72.498 | 21.797 | 20.444 | 1.00 | 48.54 | O |
| ATOM | 5542 | N | THR | B | 1157 | −71.408 | 21.033 | 22.294 | 1.00 | 52.13 | N |
| ATOM | 5543 | CA | THR | B | 1157 | −70.238 | 20.522 | 21.608 | 1.00 | 46.76 | C |
| ATOM | 5544 | CB | THR | B | 1157 | −69.000 | 21.373 | 21.913 | 1.00 | 45.98 | C |
| ATOM | 5545 | OG1 | THR | B | 1157 | −68.865 | 21.518 | 23.332 | 1.00 | 45.24 | O |
| ATOM | 5546 | CG2 | THR | B | 1157 | −69.125 | 22.750 | 21.273 | 1.00 | 51.76 | C |
| ATOM | 5547 | C | THR | B | 1157 | −69.966 | 19.101 | 22.063 | 1.00 | 41.48 | C |
| ATOM | 5548 | O | THR | B | 1157 | −70.637 | 18.584 | 22.923 | 1.00 | 43.44 | O |
| ATOM | 5549 | N | TRP | B | 1158 | −69.010 | 18.450 | 21.418 | 1.00 | 41.98 | N |
| ATOM | 5550 | CA | TRP | B | 1158 | −68.665 | 17.083 | 21.748 | 1.00 | 44.04 | C |
| ATOM | 5551 | CB | TRP | B | 1158 | −68.318 | 16.304 | 20.477 | 1.00 | 33.51 | C |
| ATOM | 5552 | CG | TRP | B | 1158 | −69.388 | 16.288 | 19.432 | 1.00 | 35.14 | C |
| ATOM | 5553 | CD1 | TRP | B | 1158 | −69.437 | 17.047 | 18.301 | 1.00 | 39.79 | C |
| ATOM | 5554 | NE1 | TRP | B | 1158 | −70.565 | 16.747 | 17.577 | 1.00 | 39.85 | N |
| ATOM | 5555 | CE2 | TRP | B | 1158 | −71.271 | 15.778 | 18.239 | 1.00 | 38.47 | C |
| ATOM | 5556 | CD2 | TRP | B | 1158 | −70.558 | 15.462 | 19.412 | 1.00 | 41.07 | C |
| ATOM | 5557 | CE3 | TRP | B | 1158 | −71.075 | 14.490 | 20.272 | 1.00 | 41.32 | C |
| ATOM | 5558 | CZ3 | TRP | B | 1158 | −72.266 | 13.876 | 19.937 | 1.00 | 39.33 | C |
| ATOM | 5559 | CH2 | TRP | B | 1158 | −72.946 | 14.211 | 18.764 | 1.00 | 41.57 | C |
| ATOM | 5560 | CZ2 | TRP | B | 1158 | −72.468 | 15.158 | 17.905 | 1.00 | 38.88 | C |
| ATOM | 5561 | C | TRP | B | 1158 | −67.497 | 16.989 | 22.701 | 1.00 | 45.63 | C |
| ATOM | 5562 | O | TRP | B | 1158 | −66.766 | 16.034 | 22.690 | 1.00 | 40.76 | O |
| ATOM | 5563 | N | ASP | B | 1159 | −67.365 | 17.975 | 23.553 | 1.00 | 47.95 | N |
| ATOM | 5564 | CA | ASP | B | 1159 | −66.298 | 18.050 | 24.529 | 1.00 | 50.02 | C |
| ATOM | 5565 | CB | ASP | B | 1159 | −66.474 | 19.280 | 25.423 | 1.00 | 65.39 | C |
| ATOM | 5566 | CG | ASP | B | 1159 | −66.303 | 20.578 | 24.667 | 1.00 | 72.54 | C |
| ATOM | 5567 | OD1 | ASP | B | 1159 | −66.286 | 20.536 | 23.417 | 1.00 | 66.52 | O |
| ATOM | 5568 | OD2 | ASP | B | 1159 | −66.196 | 21.639 | 25.321 | 1.00 | 80.29 | O |
| ATOM | 5569 | C | ASP | B | 1159 | −66.156 | 16.801 | 25.411 | 1.00 | 47.03 | C |
| ATOM | 5570 | O | ASP | B | 1159 | −65.074 | 16.286 | 25.568 | 1.00 | 46.16 | O |
| ATOM | 5571 | N | ALA | B | 1160 | −67.248 | 16.364 | 26.025 | 1.00 | 48.70 | N |
| ATOM | 5572 | CA | ALA | B | 1160 | −67.247 | 15.232 | 26.934 | 1.00 | 40.50 | C |
| ATOM | 5573 | CB | ALA | B | 1160 | −68.648 | 14.998 | 27.489 | 1.00 | 41.60 | C |
| ATOM | 5574 | C | ALA | B | 1160 | −66.700 | 13.949 | 26.317 | 1.00 | 44.47 | C |
| ATOM | 5575 | O | ALA | B | 1160 | −65.778 | 13.325 | 26.819 | 1.00 | 54.78 | O |
| ATOM | 5576 | N | TYR | B | 1161 | −67.241 | 13.600 | 25.164 | 1.00 | 42.98 | N |
| ATOM | 5577 | CA | TYR | B | 1161 | −66.800 | 12.430 | 24.424 | 1.00 | 41.50 | C |
| ATOM | 5578 | CB | TYR | B | 1161 | −67.854 | 12.016 | 23.385 | 1.00 | 40.25 | C |
| ATOM | 5579 | CG | TYR | B | 1161 | −69.182 | 11.646 | 24.020 | 1.00 | 35.93 | C |
| ATOM | 5580 | CD1 | TYR | B | 1161 | −69.458 | 10.336 | 24.388 | 1.00 | 40.37 | C |
| ATOM | 5581 | CE1 | TYR | B | 1161 | −70.662 | 9.996 | 24.987 | 1.00 | 40.96 | C |
| ATOM | 5582 | CZ | TYR | B | 1161 | −71.604 | 10.971 | 25.232 | 1.00 | 39.51 | C |
| ATOM | 5583 | OH | TYR | B | 1161 | −72.797 | 10.630 | 25.826 | 1.00 | 40.41 | O |
| ATOM | 5584 | CE2 | TYR | B | 1161 | −71.354 | 12.280 | 24.878 | 1.00 | 43.63 | C |
| ATOM | 5585 | CD2 | TYR | B | 1161 | −70.147 | 12.612 | 24.277 | 1.00 | 38.57 | C |
| ATOM | 5586 | C | TYR | B | 1161 | −65.408 | 12.579 | 23.811 | 1.00 | 45.03 | C |
| ATOM | 5587 | O | TYR | B | 1161 | −64.613 | 11.670 | 23.833 | 1.00 | 43.94 | O |
| ATOM | 5588 | N | ASP | B | 224 | −65.124 | 13.740 | 23.230 | 1.00 | 50.27 | N |
| ATOM | 5589 | CA | ASP | B | 224 | −63.812 | 14.044 | 22.642 | 1.00 | 45.76 | C |
| ATOM | 5590 | CB | ASP | B | 224 | −63.805 | 15.406 | 21.935 | 1.00 | 43.46 | C |
| ATOM | 5591 | CG | ASP | B | 224 | −64.349 | 15.333 | 20.509 | 1.00 | 42.65 | C |
| ATOM | 5592 | OD1 | ASP | B | 224 | −64.779 | 14.239 | 20.080 | 1.00 | 39.03 | O |
| ATOM | 5593 | OD2 | ASP | B | 224 | −64.357 | 16.376 | 19.818 | 1.00 | 34.28 | O |
| ATOM | 5594 | C | ASP | B | 224 | −62.705 | 13.971 | 23.664 | 1.00 | 43.70 | C |
| ATOM | 5595 | O | ASP | B | 224 | −61.638 | 13.467 | 23.400 | 1.00 | 45.92 | O |
| ATOM | 5596 | N | ARG | B | 225 | −62.997 | 14.448 | 24.855 | 1.00 | 43.63 | N |
| ATOM | 5597 | CA | ARG | B | 225 | −62.088 | 14.365 | 25.973 | 1.00 | 47.79 | C |
| ATOM | 5598 | CB | ARG | B | 225 | −62.740 | 14.948 | 27.226 | 1.00 | 48.10 | C |
| ATOM | 5599 | CG | ARG | B | 225 | −61.911 | 14.847 | 28.487 | 1.00 | 71.45 | C |
| ATOM | 5600 | CD | ARG | B | 225 | −62.621 | 15.531 | 29.657 | 1.00 | 78.76 | C |
| ATOM | 5601 | NE | ARG | B | 225 | −61.911 | 15.310 | 30.918 | 1.00 | 104.96 | N |
| ATOM | 5602 | CZ | ARG | B | 225 | −62.505 | 15.058 | 32.083 | 1.00 | 104.48 | C |
| ATOM | 5603 | NH1 | ARG | B | 225 | −63.827 | 15.007 | 32.153 | 1.00 | 106.95 | N |
| ATOM | 5604 | NH2 | ARG | B | 225 | −61.783 | 14.857 | 33.181 | 1.00 | 102.23 | N |
| ATOM | 5605 | C | ARG | B | 225 | −61.642 | 12.924 | 26.231 | 1.00 | 51.74 | C |
| ATOM | 5606 | O | ARG | B | 225 | −60.462 | 12.641 | 26.361 | 1.00 | 54.44 | O |
| ATOM | 5607 | N | LEU | B | 226 | −62.587 | 11.991 | 26.201 | 1.00 | 48.01 | N |
| ATOM | 5608 | CA | LEU | B | 226 | −62.242 | 10.574 | 26.313 | 1.00 | 45.75 | C |
| ATOM | 5609 | CB | LEU | B | 226 | −63.505 | 9.717 | 26.424 | 1.00 | 32.58 | C |
| ATOM | 5610 | CG | LEU | B | 226 | −64.327 | 9.861 | 27.701 | 1.00 | 44.06 | C |
| ATOM | 5611 | CD1 | LEU | B | 226 | −65.571 | 8.997 | 27.617 | 1.00 | 55.56 | C |
| ATOM | 5612 | CD2 | LEU | B | 226 | −63.499 | 9.492 | 28.920 | 1.00 | 46.74 | C |
| ATOM | 5613 | C | LEU | B | 226 | −61.388 | 10.047 | 25.155 | 1.00 | 50.22 | C |
| ATOM | 5614 | O | LEU | B | 226 | −60.462 | 9.286 | 25.361 | 1.00 | 50.48 | O |
| ATOM | 5615 | N | ARG | B | 227 | −61.768 | 10.374 | 23.923 | 1.00 | 44.46 | N |
| ATOM | 5616 | CA | ARG | B | 227 | −61.100 | 9.832 | 22.733 | 1.00 | 42.30 | C |
| ATOM | 5617 | CB | ARG | B | 227 | −61.888 | 10.187 | 21.472 | 1.00 | 40.14 | C |
| ATOM | 5618 | CG | ARG | B | 227 | −63.311 | 9.660 | 21.460 | 1.00 | 46.72 | C |
| ATOM | 5619 | CD | ARG | B | 227 | −64.092 | 10.214 | 20.280 | 1.00 | 47.16 | C |
| ATOM | 5620 | NE | ARG | B | 227 | −63.640 | 9.671 | 19.004 | 1.00 | 45.06 | N |
| ATOM | 5621 | CZ | ARG | B | 227 | −63.928 | 10.210 | 17.824 | 1.00 | 42.12 | C |
| ATOM | 5622 | NH1 | ARG | B | 227 | −64.658 | 11.317 | 17.760 | 1.00 | 39.78 | N |
| ATOM | 5623 | NH2 | ARG | B | 227 | −63.480 | 9.650 | 16.710 | 1.00 | 38.75 | N |
| ATOM | 5624 | C | ARG | B | 227 | −59.659 | 10.315 | 22.589 | 1.00 | 50.29 | C |
| ATOM | 5625 | O | ARG | B | 227 | −58.827 | 9.679 | 21.973 | 1.00 | 52.80 | O |
| ATOM | 5626 | N | ALA | B | 228 | −59.387 | 11.474 | 23.158 | 1.00 | 51.29 | N |
| ATOM | 5627 | CA | ALA | B | 228 | −58.088 | 12.103 | 23.110 | 1.00 | 46.82 | C |
| ATOM | 5628 | CB | ALA | B | 228 | −58.128 | 13.462 | 23.802 | 1.00 | 53.19 | C |
| ATOM | 5629 | C | ALA | B | 228 | −57.040 | 11.225 | 23.731 | 1.00 | 41.61 | C |
| ATOM | 5630 | O | ALA | B | 228 | −55.941 | 11.113 | 23.242 | 1.00 | 41.69 | O |
| ATOM | 5631 | N | TRP | B | 229 | −57.417 | 10.566 | 24.802 | 1.00 | 44.52 | N |
| ATOM | 5632 | CA | TRP | B | 229 | −56.530 | 9.632 | 25.475 | 1.00 | 45.07 | C |
| ATOM | 5633 | CB | TRP | B | 229 | −57.167 | 9.018 | 26.738 | 1.00 | 48.57 | C |
| ATOM | 5634 | CG | TRP | B | 229 | −57.158 | 9.992 | 27.895 | 1.00 | 54.96 | C |
| ATOM | 5635 | CD1 | TRP | B | 229 | −58.221 | 10.685 | 28.399 | 1.00 | 64.66 | C |
| ATOM | 5636 | NE1 | TRP | B | 229 | −57.809 | 11.505 | 29.424 | 1.00 | 66.56 | N |
| ATOM | 5637 | CE2 | TRP | B | 229 | −56.457 | 11.360 | 29.594 | 1.00 | 63.14 | C |
| ATOM | 5638 | CD2 | TRP | B | 229 | −56.011 | 10.417 | 28.647 | 1.00 | 56.25 | C |
| ATOM | 5639 | CE3 | TRP | B | 229 | −54.651 | 10.088 | 28.613 | 1.00 | 56.91 | C |
| ATOM | 5640 | CZ3 | TRP | B | 229 | −53.794 | 10.700 | 29.516 | 1.00 | 53.32 | C |
| ATOM | 5641 | CH2 | TRP | B | 229 | −54.273 | 11.634 | 30.447 | 1.00 | 60.26 | C |
| ATOM | 5642 | CZ2 | TRP | B | 229 | −55.597 | 11.974 | 30.500 | 1.00 | 67.69 | C |
| ATOM | 5643 | C | TRP | B | 229 | −55.944 | 8.578 | 24.524 | 1.00 | 44.38 | C |
| ATOM | 5644 | O | TRP | B | 229 | −54.780 | 8.246 | 24.586 | 1.00 | 54.18 | O |
| ATOM | 5645 | N | MET | B | 230 | −56.731 | 8.071 | 23.612 | 1.00 | 44.11 | N |
| ATOM | 5646 | CA | MET | B | 230 | −56.224 | 7.105 | 22.644 | 1.00 | 49.10 | C |
| ATOM | 5647 | CB | MET | B | 230 | −57.338 | 6.606 | 21.722 | 1.00 | 55.45 | C |
| ATOM | 5648 | CG | MET | B | 230 | −56.893 | 5.485 | 20.798 | 1.00 | 64.20 | C |
| ATOM | 5649 | SD | MET | B | 230 | −56.190 | 4.112 | 21.733 | 1.00 | 84.00 | S |
| ATOM | 5650 | CE | MET | B | 230 | −57.533 | 3.786 | 22.879 | 1.00 | 83.36 | C |
| ATOM | 5651 | C | MET | B | 230 | −55.052 | 7.653 | 21.809 | 1.00 | 48.68 | C |
| ATOM | 5652 | O | MET | B | 230 | −54.038 | 7.002 | 21.644 | 1.00 | 47.91 | O |
| ATOM | 5653 | N | PHE | B | 231 | −55.192 | 8.893 | 21.357 | 1.00 | 47.21 | N |
| ATOM | 5654 | CA | PHE | B | 231 | −54.148 | 9.599 | 20.609 | 1.00 | 42.14 | C |
| ATOM | 5655 | CB | PHE | B | 231 | −54.717 | 10.841 | 19.923 | 1.00 | 43.15 | C |
| ATOM | 5656 | CG | PHE | B | 231 | −55.726 | 10.515 | 18.862 | 1.00 | 43.91 | C |
| ATOM | 5657 | CD1 | PHE | B | 231 | −55.321 | 10.140 | 17.591 | 1.00 | 44.05 | C |
| ATOM | 5658 | CE1 | PHE | B | 231 | −56.251 | 9.822 | 16.619 | 1.00 | 49.83 | C |
| ATOM | 5659 | CZ | PHE | B | 231 | −57.601 | 9.868 | 16.919 | 1.00 | 56.31 | C |
| ATOM | 5660 | CE2 | PHE | B | 231 | −58.012 | 10.228 | 18.185 | 1.00 | 49.56 | C |
| ATOM | 5661 | CD2 | PHE | B | 231 | −57.079 | 10.544 | 19.147 | 1.00 | 40.55 | C |
| ATOM | 5662 | C | PHE | B | 231 | −52.897 | 9.915 | 21.449 | 1.00 | 43.33 | C |
| ATOM | 5663 | O | PHE | B | 231 | −51.789 | 9.920 | 20.956 | 1.00 | 42.69 | O |
| ATOM | 5664 | N | ILE | B | 232 | −53.097 | 10.188 | 22.733 | 1.00 | 44.56 | N |
| ATOM | 5665 | CA | ILE | B | 232 | −52.011 | 10.435 | 23.671 | 1.00 | 41.74 | C |
| ATOM | 5666 | CB | ILE | B | 232 | −52.561 | 10.932 | 25.013 | 1.00 | 37.32 | C |
| ATOM | 5667 | CG1 | ILE | B | 232 | −53.090 | 12.356 | 24.839 | 1.00 | 40.15 | C |
| ATOM | 5668 | CD1 | ILE | B | 232 | −53.872 | 12.870 | 26.019 | 1.00 | 53.68 | C |
| ATOM | 5669 | CG2 | ILE | B | 232 | −51.494 | 10.881 | 26.096 | 1.00 | 38.08 | C |
| ATOM | 5670 | C | ILE | B | 232 | −51.129 | 9.214 | 23.862 | 1.00 | 43.36 | C |
| ATOM | 5671 | O | ILE | B | 232 | −49.920 | 9.309 | 23.847 | 1.00 | 44.21 | O |
| ATOM | 5672 | N | CYS | B | 233 | −51.723 | 8.039 | 23.975 | 1.00 | 43.23 | N |
| ATOM | 5673 | CA | CYS | B | 233 | −50.924 | 6.823 | 23.989 | 1.00 | 45.68 | C |
| ATOM | 5674 | CB | CYS | B | 233 | −51.799 | 5.580 | 24.165 | 1.00 | 43.04 | C |
| ATOM | 5675 | SG | CYS | B | 233 | −52.187 | 5.211 | 25.885 | 1.00 | 71.29 | S |
| ATOM | 5676 | C | CYS | B | 233 | −50.037 | 6.665 | 22.782 | 1.00 | 41.51 | C |
| ATOM | 5677 | O | CYS | B | 233 | −48.910 | 6.292 | 22.897 | 1.00 | 40.89 | O |
| ATOM | 5678 | N | ILE | B | 234 | −50.557 | 6.921 | 21.612 | 1.00 | 39.87 | N |
| ATOM | 5679 | CA | ILE | B | 234 | −49.787 | 6.732 | 20.410 | 1.00 | 39.29 | C |
| ATOM | 5680 | CB | ILE | B | 234 | −50.673 | 6.777 | 19.150 | 1.00 | 45.01 | C |
| ATOM | 5681 | CG1 | ILE | B | 234 | −51.745 | 5.688 | 19.207 | 1.00 | 43.15 | C |
| ATOM | 5682 | CD1 | ILE | B | 234 | −52.621 | 5.648 | 17.982 | 1.00 | 38.11 | C |
| ATOM | 5683 | CG2 | ILE | B | 234 | −49.824 | 6.626 | 17.895 | 1.00 | 36.86 | C |
| ATOM | 5684 | C | ILE | B | 234 | −48.721 | 7.793 | 20.301 | 1.00 | 39.23 | C |
| ATOM | 5685 | O | ILE | B | 234 | −47.556 | 7.520 | 20.176 | 1.00 | 40.68 | O |
| ATOM | 5686 | N | GLY | B | 235 | −49.128 | 9.018 | 20.440 | 1.00 | 42.49 | N |
| ATOM | 5687 | CA | GLY | B | 235 | −48.243 | 10.119 | 20.382 | 1.00 | 40.13 | C |
| ATOM | 5688 | C | GLY | B | 235 | −47.091 | 10.142 | 21.323 | 1.00 | 38.70 | C |
| ATOM | 5689 | O | GLY | B | 235 | −46.005 | 10.461 | 20.897 | 1.00 | 40.49 | O |
| ATOM | 5690 | N | TRP | B | 236 | −47.312 | 9.939 | 22.613 | 1.00 | 35.46 | N |
| ATOM | 5691 | CA | TRP | B | 236 | −46.241 | 10.113 | 23.557 | 1.00 | 38.33 | C |
| ATOM | 5692 | CB | TRP | B | 236 | −46.690 | 11.041 | 24.688 | 1.00 | 36.55 | C |
| ATOM | 5693 | CG | TRP | B | 236 | −47.132 | 12.413 | 24.322 | 1.00 | 38.70 | C |
| ATOM | 5694 | CD1 | TRP | B | 236 | −48.413 | 12.870 | 24.264 | 1.00 | 32.89 | C |
| ATOM | 5695 | NE1 | TRP | B | 236 | −48.426 | 14.200 | 23.923 | 1.00 | 35.36 | N |
| ATOM | 5696 | CE2 | TRP | B | 236 | −47.134 | 14.628 | 23.767 | 1.00 | 40.51 | C |
| ATOM | 5697 | CD2 | TRP | B | 236 | −46.291 | 13.530 | 24.021 | 1.00 | 43.95 | C |
| ATOM | 5698 | CE3 | TRP | B | 236 | −44.908 | 13.706 | 23.927 | 1.00 | 40.16 | C |
| ATOM | 5699 | CZ3 | TRP | B | 236 | −44.422 | 14.953 | 23.590 | 1.00 | 40.03 | C |
| ATOM | 5700 | CH2 | TRP | B | 236 | −45.287 | 16.025 | 23.343 | 1.00 | 41.51 | C |
| ATOM | 5701 | CZ2 | TRP | B | 236 | −46.643 | 15.881 | 23.427 | 1.00 | 43.79 | C |
| ATOM | 5702 | C | TRP | B | 236 | −45.917 | 8.831 | 24.251 | 1.00 | 36.78 | C |
| ATOM | 5703 | O | TRP | B | 236 | −44.780 | 8.513 | 24.498 | 1.00 | 44.89 | O |
| ATOM | 5704 | N | GLY | B | 237 | −46.920 | 8.072 | 24.559 | 1.00 | 40.77 | N |
| ATOM | 5705 | CA | GLY | B | 237 | −46.622 | 6.904 | 25.296 | 1.00 | 42.05 | C |
| ATOM | 5706 | C | GLY | B | 237 | −46.049 | 5.764 | 24.557 | 1.00 | 41.38 | C |
| ATOM | 5707 | O | GLY | B | 237 | −45.326 | 4.993 | 25.147 | 1.00 | 47.32 | O |
| ATOM | 5708 | N | VAL | B | 238 | −46.347 | 5.591 | 23.296 | 1.00 | 40.40 | N |
| ATOM | 5709 | CA | VAL | B | 238 | −45.891 | 4.410 | 22.619 | 1.00 | 41.28 | C |
| ATOM | 5710 | CB | VAL | B | 238 | −46.744 | 4.118 | 21.340 | 1.00 | 41.18 | C |
| ATOM | 5711 | CG1 | VAL | B | 238 | −46.041 | 3.149 | 20.402 | 1.00 | 37.33 | C |
| ATOM | 5712 | CG2 | VAL | B | 238 | −48.098 | 3.564 | 21.714 | 1.00 | 40.59 | C |
| ATOM | 5713 | C | VAL | B | 238 | −44.445 | 4.538 | 22.236 | 1.00 | 38.22 | C |
| ATOM | 5714 | O | VAL | B | 238 | −43.777 | 3.532 | 22.297 | 1.00 | 39.05 | O |
| ATOM | 5715 | N | PRO | B | 239 | −43.819 | 5.677 | 21.913 | 1.00 | 39.47 | N |
| ATOM | 5716 | CA | PRO | B | 239 | −42.415 | 5.536 | 21.652 | 1.00 | 41.66 | C |
| ATOM | 5717 | CB | PRO | B | 239 | −42.142 | 6.702 | 20.700 | 1.00 | 42.25 | C |
| ATOM | 5718 | CG | PRO | B | 239 | −43.100 | 7.757 | 21.133 | 1.00 | 42.54 | C |
| ATOM | 5719 | CD | PRO | B | 239 | −44.332 | 6.997 | 21.521 | 1.00 | 42.01 | C |
| ATOM | 5720 | C | PRO | B | 239 | −41.495 | 5.627 | 22.822 | 1.00 | 40.32 | C |
| ATOM | 5721 | O | PRO | B | 239 | −40.335 | 5.349 | 22.676 | 1.00 | 38.74 | O |
| ATOM | 5722 | N | PHE | B | 240 | −42.064 | 5.785 | 23.993 | 1.00 | 39.39 | N |
| ATOM | 5723 | CA | PHE | B | 240 | −41.351 | 5.571 | 25.212 | 1.00 | 43.56 | C |
| ATOM | 5724 | CB | PHE | B | 240 | −42.154 | 5.951 | 26.463 | 1.00 | 37.49 | C |
| ATOM | 5725 | CG | PHE | B | 240 | −41.293 | 6.130 | 27.676 | 1.00 | 35.34 | C |
| ATOM | 5726 | CD1 | PHE | B | 240 | −40.487 | 7.248 | 27.811 | 1.00 | 44.99 | C |
| ATOM | 5727 | CE1 | PHE | B | 240 | −39.672 | 7.403 | 28.918 | 1.00 | 43.23 | C |
| ATOM | 5728 | CZ | PHE | B | 240 | −39.653 | 6.433 | 29.895 | 1.00 | 40.03 | C |
| ATOM | 5729 | CE2 | PHE | B | 240 | −40.446 | 5.317 | 29.766 | 1.00 | 40.33 | C |
| ATOM | 5730 | CD2 | PHE | B | 240 | −41.255 | 5.165 | 28.659 | 1.00 | 36.10 | C |
| ATOM | 5731 | C | PHE | B | 240 | −40.670 | 4.206 | 25.341 | 1.00 | 44.92 | C |
| ATOM | 5732 | O | PHE | B | 240 | −39.468 | 4.169 | 25.478 | 1.00 | 34.06 | O |
| ATOM | 5733 | N | PRO | B | 241 | −41.324 | 3.018 | 25.247 | 1.00 | 46.65 | N |
| ATOM | 5734 | CA | PRO | B | 241 | −40.648 | 1.736 | 25.305 | 1.00 | 36.83 | C |
| ATOM | 5735 | CB | PRO | B | 241 | −41.784 | 0.730 | 25.084 | 1.00 | 34.17 | C |
| ATOM | 5736 | CG | PRO | B | 241 | −43.004 | 1.434 | 25.512 | 1.00 | 39.52 | C |
| ATOM | 5737 | CD | PRO | B | 241 | −42.782 | 2.857 | 25.123 | 1.00 | 38.76 | C |
| ATOM | 5738 | C | PRO | B | 241 | −39.641 | 1.609 | 24.193 | 1.00 | 35.27 | C |
| ATOM | 5739 | O | PRO | B | 241 | −38.638 | 0.964 | 24.376 | 1.00 | 40.61 | O |
| ATOM | 5740 | N | ILE | B | 242 | −39.928 | 2.213 | 23.054 | 1.00 | 33.52 | N |
| ATOM | 5741 | CA | ILE | B | 242 | −39.063 | 2.172 | 21.903 | 1.00 | 35.74 | C |
| ATOM | 5742 | CB | ILE | B | 242 | −39.667 | 2.906 | 20.690 | 1.00 | 36.36 | C |
| ATOM | 5743 | CG1 | ILE | B | 242 | −40.939 | 2.198 | 20.218 | 1.00 | 31.99 | C |
| ATOM | 5744 | CD1 | ILE | B | 242 | −41.574 | 2.824 | 18.988 | 1.00 | 28.78 | C |
| ATOM | 5745 | CG2 | ILE | B | 242 | −38.651 | 2.981 | 19.554 | 1.00 | 36.21 | C |
| ATOM | 5746 | C | ILE | B | 242 | −37.712 | 2.767 | 22.217 | 1.00 | 33.91 | C |
| ATOM | 5747 | O | ILE | B | 242 | −36.710 | 2.126 | 22.061 | 1.00 | 37.14 | O |
| ATOM | 5748 | N | ILE | B | 243 | −37.702 | 3.966 | 22.740 | 1.00 | 34.15 | N |
| ATOM | 5749 | CA | ILE | B | 243 | −36.484 | 4.611 | 23.212 | 1.00 | 35.17 | C |
| ATOM | 5750 | CB | ILE | B | 243 | −36.730 | 6.083 | 23.624 | 1.00 | 41.22 | C |
| ATOM | 5751 | CG1 | ILE | B | 243 | −36.672 | 6.982 | 22.389 | 1.00 | 40.71 | C |
| ATOM | 5752 | CD1 | ILE | B | 243 | −35.335 | 6.998 | 21.701 | 1.00 | 24.60 | C |
| ATOM | 5753 | CG2 | ILE | B | 243 | −35.700 | 6.562 | 24.635 | 1.00 | 35.76 | C |
| ATOM | 5754 | C | ILE | B | 243 | −35.775 | 3.842 | 24.308 | 1.00 | 35.51 | C |
| ATOM | 5755 | O | ILE | B | 243 | −34.592 | 3.676 | 24.282 | 1.00 | 41.05 | O |
| ATOM | 5756 | N | VAL | B | 244 | −36.505 | 3.334 | 25.267 | 1.00 | 35.67 | N |
| ATOM | 5757 | CA | VAL | B | 244 | −35.921 | 2.554 | 26.338 | 1.00 | 32.44 | C |
| ATOM | 5758 | CB | VAL | B | 244 | −36.994 | 2.114 | 27.364 | 1.00 | 29.67 | C |
| ATOM | 5759 | CG1 | VAL | B | 244 | −36.410 | 1.167 | 28.393 | 1.00 | 29.33 | C |
| ATOM | 5760 | CG2 | VAL | B | 244 | −37.593 | 3.328 | 28.052 | 1.00 | 29.68 | C |
| ATOM | 5761 | C | VAL | B | 244 | −35.199 | 1.334 | 25.786 | 1.00 | 34.09 | C |
| ATOM | 5762 | O | VAL | B | 244 | −34.064 | 1.101 | 26.095 | 1.00 | 35.74 | O |
| ATOM | 5763 | N | ALA | B | 245 | −35.819 | 0.627 | 24.874 | 1.00 | 31.68 | N |
| ATOM | 5764 | CA | ALA | B | 245 | −35.157 | −0.467 | 24.185 | 1.00 | 36.71 | C |
| ATOM | 5765 | CB | ALA | B | 245 | −36.166 | −1.217 | 23.319 | 1.00 | 31.83 | C |
| ATOM | 5766 | C | ALA | B | 245 | −33.950 | −0.045 | 23.350 | 1.00 | 43.16 | C |
| ATOM | 5767 | O | ALA | B | 245 | −32.955 | −0.736 | 23.305 | 1.00 | 49.11 | O |
| ATOM | 5768 | N | TRP | B | 246 | −34.028 | 1.111 | 22.702 | 1.00 | 41.93 | N |
| ATOM | 5769 | CA | TRP | B | 246 | −32.914 | 1.637 | 21.936 | 1.00 | 37.92 | C |
| ATOM | 5770 | CB | TRP | B | 246 | −33.331 | 2.899 | 21.174 | 1.00 | 31.72 | C |
| ATOM | 5771 | CG | TRP | B | 246 | −32.193 | 3.650 | 20.551 | 1.00 | 30.38 | C |
| ATOM | 5772 | CD1 | TRP | B | 246 | −31.576 | 3.359 | 19.373 | 1.00 | 36.23 | C |
| ATOM | 5773 | NE1 | TRP | B | 246 | −30.581 | 4.267 | 19.118 | 1.00 | 34.84 | N |
| ATOM | 5774 | CE2 | TRP | B | 246 | −30.540 | 5.178 | 20.139 | 1.00 | 32.83 | C |
| ATOM | 5775 | CD2 | TRP | B | 246 | −31.546 | 4.823 | 21.061 | 1.00 | 31.26 | C |
| ATOM | 5776 | CE3 | TRP | B | 246 | −31.713 | 5.605 | 22.208 | 1.00 | 31.87 | C |
| ATOM | 5777 | CZ3 | TRP | B | 246 | −30.882 | 6.700 | 22.388 | 1.00 | 33.64 | C |
| ATOM | 5778 | CH2 | TRP | B | 246 | −29.892 | 7.022 | 21.450 | 1.00 | 32.57 | C |
| ATOM | 5779 | CZ2 | TRP | B | 246 | −29.708 | 6.275 | 20.323 | 1.00 | 31.52 | C |
| ATOM | 5780 | C | TRP | B | 246 | −31.738 | 1.945 | 22.832 | 1.00 | 41.67 | C |
| ATOM | 5781 | O | TRP | B | 246 | −30.614 | 1.647 | 22.510 | 1.00 | 41.98 | O |
| ATOM | 5782 | N | ALA | B | 247 | −32.021 | 2.579 | 23.951 | 1.00 | 36.69 | N |
| ATOM | 5783 | CA | ALA | B | 247 | −31.037 | 2.966 | 24.902 | 1.00 | 33.46 | C |
| ATOM | 5784 | CB | ALA | B | 247 | −31.672 | 3.809 | 25.996 | 1.00 | 32.59 | C |
| ATOM | 5785 | C | ALA | B | 247 | −30.370 | 1.771 | 25.487 | 1.00 | 43.21 | C |
| ATOM | 5786 | O | ALA | B | 247 | −29.182 | 1.743 | 25.632 | 1.00 | 47.16 | O |
| ATOM | 5787 | N | ILE | B | 248 | −31.142 | 0.735 | 25.769 | 1.00 | 42.33 | N |
| ATOM | 5788 | CA | ILE | B | 248 | −30.590 | −0.546 | 26.164 | 1.00 | 43.77 | C |
| ATOM | 5789 | CB | ILE | B | 248 | −31.696 | −1.567 | 26.484 | 1.00 | 42.93 | C |
| ATOM | 5790 | CG1 | ILE | B | 248 | −32.438 | −1.155 | 27.754 | 1.00 | 46.71 | C |
| ATOM | 5791 | CD1 | ILE | B | 248 | −33.598 | −2.060 | 28.102 | 1.00 | 51.01 | C |
| ATOM | 5792 | CG2 | ILE | B | 248 | −31.109 | −2.964 | 26.647 | 1.00 | 48.63 | C |
| ATOM | 5793 | C | ILE | B | 248 | −29.675 | −1.101 | 25.086 | 1.00 | 47.49 | C |
| ATOM | 5794 | O | ILE | B | 248 | −28.594 | −1.555 | 25.341 | 1.00 | 46.39 | O |
| ATOM | 5795 | N | GLY | B | 249 | −30.102 | −1.024 | 23.858 | 1.00 | 48.07 | N |
| ATOM | 5796 | CA | GLY | B | 249 | −29.317 | −1.501 | 22.764 | 1.00 | 49.53 | C |
| ATOM | 5797 | C | GLY | B | 249 | −28.024 | −0.823 | 22.569 | 1.00 | 52.15 | C |
| ATOM | 5798 | O | GLY | B | 249 | −27.026 | −1.441 | 22.295 | 1.00 | 53.33 | O |
| ATOM | 5799 | N | LYS | B | 250 | −28.010 | 0.465 | 22.757 | 1.00 | 50.52 | N |
| ATOM | 5800 | CA | LYS | B | 250 | −26.784 | 1.208 | 22.785 | 1.00 | 46.04 | C |
| ATOM | 5801 | CB | LYS | B | 250 | −27.084 | 2.710 | 22.812 | 1.00 | 45.90 | C |
| ATOM | 5802 | CG | LYS | B | 250 | −27.559 | 3.295 | 21.493 | 1.00 | 43.06 | C |
| ATOM | 5803 | CD | LYS | B | 250 | −26.385 | 3.685 | 20.609 | 1.00 | 44.51 | C |
| ATOM | 5804 | CE | LYS | B | 250 | −26.845 | 4.492 | 19.407 | 1.00 | 42.45 | C |
| ATOM | 5805 | NZ | LYS | B | 250 | −25.710 | 4.980 | 18.580 | 1.00 | 42.44 | N |
| ATOM | 5806 | C | LYS | B | 250 | −25.945 | 0.846 | 23.998 | 1.00 | 51.91 | C |
| ATOM | 5807 | O | LYS | B | 250 | −24.781 | 0.641 | 23.922 | 1.00 | 59.08 | O |
| ATOM | 5808 | N | LEU | B | 251 | −26.537 | 0.761 | 25.156 | 1.00 | 51.61 | N |
| ATOM | 5809 | CA | LEU | B | 251 | −25.789 | 0.461 | 26.370 | 1.00 | 44.57 | C |
| ATOM | 5810 | CB | LEU | B | 251 | −26.754 | 0.395 | 27.552 | 1.00 | 48.22 | C |
| ATOM | 5811 | CG | LEU | B | 251 | −26.263 | 0.948 | 28.883 | 1.00 | 43.86 | C |
| ATOM | 5812 | CD1 | LEU | B | 251 | −25.778 | 2.372 | 28.708 | 1.00 | 41.46 | C |
| ATOM | 5813 | CD2 | LEU | B | 251 | −27.388 | 0.892 | 29.898 | 1.00 | 53.66 | C |
| ATOM | 5814 | C | LEU | B | 251 | −24.981 | −0.831 | 26.283 | 1.00 | 42.22 | C |
| ATOM | 5815 | O | LEU | B | 251 | −23.797 | −0.838 | 26.510 | 1.00 | 51.44 | O |
| ATOM | 5816 | N | TYR | B | 252 | −25.614 | −1.890 | 25.811 | 1.00 | 46.21 | N |
| ATOM | 5817 | CA | TYR | B | 252 | −24.948 | −3.136 | 25.404 | 1.00 | 53.02 | C |
| ATOM | 5818 | CB | TYR | B | 252 | −26.020 | −4.200 | 25.144 | 1.00 | 64.55 | C |
| ATOM | 5819 | CG | TYR | B | 252 | −26.714 | −4.749 | 26.371 | 1.00 | 72.13 | C |
| ATOM | 5820 | CD1 | TYR | B | 252 | −26.054 | −4.848 | 27.588 | 1.00 | 68.58 | C |
| ATOM | 5821 | CE1 | TYR | B | 252 | −26.689 | −5.360 | 28.705 | 1.00 | 68.03 | C |
| ATOM | 5822 | CZ | TYR | B | 252 | −27.998 | −5.783 | 28.608 | 1.00 | 79.92 | C |
| ATOM | 5823 | OH | TYR | B | 252 | −28.639 | −6.298 | 29.709 | 1.00 | 94.48 | O |
| ATOM | 5824 | CE2 | TYR | B | 252 | −28.673 | −5.698 | 27.409 | 1.00 | 77.07 | C |
| ATOM | 5825 | CD2 | TYR | B | 252 | −28.032 | −5.186 | 26.303 | 1.00 | 73.35 | C |
| ATOM | 5826 | C | TYR | B | 252 | −24.010 | −3.150 | 24.185 | 1.00 | 55.83 | C |
| ATOM | 5827 | O | TYR | B | 252 | −22.877 | −3.557 | 24.295 | 1.00 | 61.38 | O |
| ATOM | 5828 | N | TYR | B | 253 | −24.494 | −2.768 | 23.020 | 1.00 | 54.37 | N |
| ATOM | 5829 | CA | TYR | B | 253 | −23.769 | −2.992 | 21.769 | 1.00 | 58.63 | C |
| ATOM | 5830 | CB | TYR | B | 253 | −24.753 | −3.463 | 20.688 | 1.00 | 69.88 | C |
| ATOM | 5831 | CG | TYR | B | 253 | −25.519 | −4.735 | 20.986 | 1.00 | 81.26 | C |
| ATOM | 5832 | CD2 | TYR | B | 253 | −26.747 | −4.693 | 21.629 | 1.00 | 76.67 | C |
| ATOM | 5833 | CE2 | TYR | B | 253 | −27.458 | −5.851 | 21.889 | 1.00 | 93.13 | C |
| ATOM | 5834 | CZ | TYR | B | 253 | −26.948 | −7.072 | 21.492 | 1.00 | 104.24 | C |
| ATOM | 5835 | OH | TYR | B | 253 | −27.650 | −8.230 | 21.744 | 1.00 | 110.23 | O |
| ATOM | 5836 | CE1 | TYR | B | 253 | −25.736 | −7.140 | 20.842 | 1.00 | 100.71 | C |
| ATOM | 5837 | CD1 | TYR | B | 253 | −25.031 | −5.975 | 20.587 | 1.00 | 96.50 | C |
| ATOM | 5838 | C | TYR | B | 253 | −22.943 | −1.868 | 21.136 | 1.00 | 58.31 | C |
| ATOM | 5839 | O | TYR | B | 253 | −21.866 | −2.076 | 20.638 | 1.00 | 58.15 | O |
| ATOM | 5840 | N | ASP | B | 254 | −23.509 | −0.682 | 21.098 | 1.00 | 62.15 | N |
| ATOM | 5841 | CA | ASP | B | 254 | −22.864 | 0.481 | 20.566 | 1.00 | 51.75 | C |
| ATOM | 5842 | CB | ASP | B | 254 | −23.669 | 1.012 | 19.379 | 1.00 | 51.49 | C |
| ATOM | 5843 | CG | ASP | B | 254 | −23.051 | 2.244 | 18.755 | 1.00 | 59.21 | C |
| ATOM | 5844 | OD1 | ASP | B | 254 | −21.927 | 2.618 | 19.142 | 1.00 | 70.94 | O |
| ATOM | 5845 | OD2 | ASP | B | 254 | −23.687 | 2.837 | 17.860 | 1.00 | 57.96 | O |
| ATOM | 5846 | C | ASP | B | 254 | −22.931 | 1.421 | 21.705 | 1.00 | 45.52 | C |
| ATOM | 5847 | O | ASP | B | 254 | −23.993 | 1.765 | 22.132 | 1.00 | 54.19 | O |
| ATOM | 5848 | N | ASN | B | 255 | −21.772 | 1.770 | 22.260 | 1.00 | 42.02 | N |
| ATOM | 5849 | CA | ASN | B | 255 | −21.694 | 2.599 | 23.465 | 1.00 | 43.04 | C |
| ATOM | 5850 | CB | ASN | B | 255 | −21.271 | 1.779 | 24.690 | 1.00 | 45.56 | C |
| ATOM | 5851 | CG | ASN | B | 255 | −21.852 | 2.331 | 25.988 | 1.00 | 42.55 | C |
| ATOM | 5852 | OD1 | ASN | B | 255 | −22.753 | 3.171 | 25.971 | 1.00 | 36.05 | O |
| ATOM | 5853 | ND2 | ASN | B | 255 | −21.337 | 1.860 | 27.117 | 1.00 | 48.04 | N |
| ATOM | 5854 | C | ASN | B | 255 | −20.903 | 3.910 | 23.361 | 1.00 | 51.03 | C |
| ATOM | 5855 | O | ASN | B | 255 | −20.442 | 4.429 | 24.378 | 1.00 | 47.33 | O |
| ATOM | 5856 | N | GLU | B | 256 | −20.721 | 4.438 | 22.154 | 1.00 | 53.90 | N |
| ATOM | 5857 | CA | GLU | B | 256 | −19.757 | 5.522 | 21.950 | 1.00 | 51.10 | C |
| ATOM | 5858 | CB | GLU | B | 256 | −18.521 | 5.001 | 21.192 | 1.00 | 55.15 | C |
| ATOM | 5859 | CG | GLU | B | 256 | −18.823 | 3.950 | 20.119 | 1.00 | 56.35 | C |
| ATOM | 5860 | CD | GLU | B | 256 | −17.760 | 3.877 | 19.025 | 1.00 | 75.39 | C |
| ATOM | 5861 | OE1 | GLU | B | 256 | −16.625 | 3.441 | 19.316 | 1.00 | 86.12 | O |
| ATOM | 5862 | OE2 | GLU | B | 256 | −18.059 | 4.254 | 17.870 | 1.00 | 71.43 | O |
| ATOM | 5863 | C | GLU | B | 256 | −20.280 | 6.795 | 21.275 | 1.00 | 55.07 | C |
| ATOM | 5864 | O | GLU | B | 256 | −21.300 | 6.782 | 20.585 | 1.00 | 59.33 | O |
| ATOM | 5865 | N | LYS | B | 257 | −19.551 | 7.890 | 21.489 | 1.00 | 55.94 | N |
| ATOM | 5866 | CA | LYS | B | 257 | −19.851 | 9.188 | 20.881 | 1.00 | 54.38 | C |
| ATOM | 5867 | CB | LYS | B | 257 | −19.655 | 9.120 | 19.359 | 1.00 | 56.47 | C |
| ATOM | 5868 | CG | LYS | B | 257 | −18.283 | 8.611 | 18.921 | 1.00 | 50.44 | C |
| ATOM | 5869 | CD | LYS | B | 257 | −18.216 | 8.407 | 17.409 | 1.00 | 52.17 | C |
| ATOM | 5870 | CE | LYS | B | 257 | −18.187 | 9.732 | 16.654 | 1.00 | 48.69 | C |
| ATOM | 5871 | NZ | LYS | B | 257 | −18.347 | 9.553 | 15.180 | 1.00 | 40.54 | N |
| ATOM | 5872 | C | LYS | B | 257 | −21.227 | 9.764 | 21.214 | 1.00 | 51.95 | C |
| ATOM | 5873 | O | LYS | B | 257 | −21.900 | 10.317 | 20.344 | 1.00 | 58.85 | O |
| ATOM | 5874 | N | CYS | B | 258 | −21.644 | 9.632 | 22.469 | 1.00 | 50.76 | N |
| ATOM | 5875 | CA | CYS | B | 258 | −22.953 | 10.133 | 22.887 | 1.00 | 49.22 | C |
| ATOM | 5876 | CB | CYS | B | 258 | −23.067 | 11.648 | 22.712 | 1.00 | 47.35 | C |
| ATOM | 5877 | SG | CYS | B | 258 | −21.689 | 12.529 | 23.449 | 1.00 | 57.71 | S |
| ATOM | 5878 | C | CYS | B | 258 | −24.146 | 9.419 | 22.299 | 1.00 | 49.32 | C |
| ATOM | 5879 | O | CYS | B | 258 | −25.215 | 9.976 | 22.239 | 1.00 | 45.52 | O |
| ATOM | 5880 | N | TRP | B | 259 | −23.945 | 8.210 | 21.798 | 1.00 | 56.20 | N |
| ATOM | 5881 | CA | TRP | B | 259 | −25.006 | 7.480 | 21.164 | 1.00 | 49.20 | C |
| ATOM | 5882 | CB | TRP | B | 259 | −26.224 | 7.285 | 22.084 | 1.00 | 45.80 | C |
| ATOM | 5883 | CG | TRP | B | 259 | −25.963 | 6.441 | 23.297 | 1.00 | 44.85 | C |
| ATOM | 5884 | CD1 | TRP | B | 259 | −24.827 | 5.744 | 23.590 | 1.00 | 44.23 | C |
| ATOM | 5885 | NE1 | TRP | B | 259 | −24.973 | 5.084 | 24.785 | 1.00 | 42.62 | N |
| ATOM | 5886 | CE2 | TRP | B | 259 | −26.220 | 5.348 | 25.286 | 1.00 | 47.28 | C |
| ATOM | 5887 | CD2 | TRP | B | 259 | −26.870 | 6.202 | 24.375 | 1.00 | 44.85 | C |
| ATOM | 5888 | CE3 | TRP | B | 259 | −28.170 | 6.627 | 24.659 | 1.00 | 38.10 | C |
| ATOM | 5889 | CZ3 | TRP | B | 259 | −28.768 | 6.193 | 25.824 | 1.00 | 37.79 | C |
| ATOM | 5890 | CH2 | TRP | B | 259 | −28.094 | 5.346 | 26.711 | 1.00 | 39.61 | C |
| ATOM | 5891 | CZ2 | TRP | B | 259 | −26.823 | 4.915 | 26.460 | 1.00 | 44.11 | C |
| ATOM | 5892 | C | TRP | B | 259 | −25.394 | 8.078 | 19.809 | 1.00 | 53.49 | C |
| ATOM | 5893 | O | TRP | B | 259 | −26.464 | 7.833 | 19.272 | 1.00 | 60.33 | O |
| ATOM | 5894 | N | ALA | B | 260 | −24.531 | 8.953 | 19.321 | 1.00 | 49.71 | N |
| ATOM | 5895 | CA | ALA | B | 260 | −24.811 | 9.743 | 18.174 | 1.00 | 52.08 | C |
| ATOM | 5896 | CB | ALA | B | 260 | −24.699 | 11.218 | 18.523 | 1.00 | 51.52 | C |
| ATOM | 5897 | C | ALA | B | 260 | −23.871 | 9.400 | 17.034 | 1.00 | 55.91 | C |
| ATOM | 5898 | O | ALA | B | 260 | −23.769 | 10.132 | 16.074 | 1.00 | 69.89 | O |
| ATOM | 5899 | N | GLY | B | 261 | −23.031 | 8.415 | 17.225 | 1.00 | 53.56 | N |
| ATOM | 5900 | CA | GLY | B | 261 | −22.054 | 8.152 | 16.213 | 1.00 | 65.85 | C |
| ATOM | 5901 | C | GLY | B | 261 | −22.433 | 7.319 | 15.046 | 1.00 | 70.85 | C |
| ATOM | 5902 | O | GLY | B | 261 | −22.871 | 6.191 | 15.206 | 1.00 | 75.40 | O |
| ATOM | 5903 | N | LYS | B | 262 | −22.081 | 7.832 | 13.848 | 1.00 | 76.17 | N |
| ATOM | 5904 | CA | LYS | B | 262 | −22.198 | 7.072 | 12.602 | 1.00 | 85.44 | C |
| ATOM | 5905 | C | LYS | B | 262 | −21.186 | 5.939 | 12.586 | 1.00 | 93.61 | C |
| ATOM | 5906 | O | LYS | B | 262 | −20.070 | 6.085 | 13.085 | 1.00 | 86.30 | O |
| ATOM | 5907 | CB | LYS | B | 262 | −21.973 | 7.974 | 11.386 | 1.00 | 82.10 | C |
| ATOM | 5908 | CG | LYS | B | 262 | −23.111 | 8.933 | 11.088 | 1.00 | 94.04 | C |
| ATOM | 5909 | CD | LYS | B | 262 | −22.867 | 9.676 | 9.781 | 1.00 | 107.52 | C |
| ATOM | 5910 | CE | LYS | B | 262 | −24.054 | 10.551 | 9.399 | 1.00 | 115.73 | C |
| ATOM | 5911 | NZ | LYS | B | 262 | −23.840 | 11.246 | 8.096 | 1.00 | 102.37 | N |
| ATOM | 5912 | O | ARG | B | 263 | −22.360 | 2.055 | 11.123 | 1.00 | 111.38 | O |
| ATOM | 5913 | N | ARG | B | 263 | −21.632 | 4.784 | 12.108 | 1.00 | 96.11 | N |
| ATOM | 5914 | CA | ARG | B | 263 | −20.798 | 3.607 | 12.101 | 1.00 | 89.37 | C |
| ATOM | 5915 | C | ARG | B | 263 | −21.277 | 2.639 | 11.005 | 1.00 | 96.81 | C |
| ATOM | 5916 | CB | ARG | B | 263 | −20.877 | 2.866 | 13.443 | 1.00 | 87.93 | C |
| ATOM | 5917 | CG | ARG | B | 263 | −20.067 | 3.447 | 14.593 | 1.00 | 86.03 | C |
| ATOM | 5918 | CD | ARG | B | 263 | −18.576 | 3.448 | 14.300 | 1.00 | 85.59 | C |
| ATOM | 5919 | NE | ARG | B | 263 | −17.797 | 3.169 | 15.504 | 1.00 | 79.88 | N |
| ATOM | 5920 | CZ | ARG | B | 263 | −16.491 | 3.390 | 15.626 | 1.00 | 90.93 | C |
| ATOM | 5921 | NH1 | ARG | B | 263 | −15.801 | 3.916 | 14.622 | 1.00 | 99.93 | N |
| ATOM | 5922 | NH2 | ARG | B | 263 | −15.876 | 3.095 | 16.763 | 1.00 | 86.92 | N |
| ATOM | 5923 | O | TYR | B | 267 | −28.619 | −2.106 | 14.686 | 1.00 | 78.63 | O |
| ATOM | 5924 | N | TYR | B | 267 | −26.256 | −1.892 | 13.029 | 1.00 | 73.33 | N |
| ATOM | 5925 | CA | TYR | B | 267 | −27.308 | −0.918 | 13.089 | 1.00 | 75.29 | C |
| ATOM | 5926 | C | TYR | B | 267 | −28.069 | −1.047 | 14.389 | 1.00 | 73.58 | C |
| ATOM | 5927 | CB | TYR | B | 267 | −28.223 | −1.084 | 11.865 | 1.00 | 71.57 | C |
| ATOM | 5928 | CG | TYR | B | 267 | −27.448 | −1.180 | 10.557 | 1.00 | 86.63 | C |
| ATOM | 5929 | CD1 | TYR | B | 267 | −27.079 | −0.033 | 9.854 | 1.00 | 95.51 | C |
| ATOM | 5930 | CD2 | TYR | B | 267 | −27.064 | −2.415 | 10.037 | 1.00 | 83.19 | C |
| ATOM | 5931 | CE1 | TYR | B | 267 | −26.357 | −0.114 | 8.666 | 1.00 | 95.29 | C |
| ATOM | 5932 | CE2 | TYR | B | 267 | −26.342 | −2.503 | 8.852 | 1.00 | 90.26 | C |
| ATOM | 5933 | CZ | TYR | B | 267 | −25.992 | −1.352 | 8.172 | 1.00 | 92.04 | C |
| ATOM | 5934 | OH | TYR | B | 267 | −25.278 | −1.432 | 6.996 | 1.00 | 82.25 | O |
| ATOM | 5935 | N | THR | B | 268 | −28.071 | 0.029 | 15.182 | 1.00 | 65.03 | N |
| ATOM | 5936 | CA | THR | B | 268 | −28.758 | 0.001 | 16.459 | 1.00 | 62.36 | C |
| ATOM | 5937 | CB | THR | B | 268 | −27.779 | 0.061 | 17.652 | 1.00 | 59.41 | C |
| ATOM | 5938 | OG1 | THR | B | 268 | −27.063 | 1.301 | 17.628 | 1.00 | 63.40 | O |
| ATOM | 5939 | CG2 | THR | B | 268 | −26.793 | −1.098 | 17.600 | 1.00 | 60.39 | C |
| ATOM | 5940 | C | THR | B | 268 | −29.705 | 1.152 | 16.545 | 1.00 | 55.69 | C |
| ATOM | 5941 | O | THR | B | 268 | −30.443 | 1.284 | 17.492 | 1.00 | 53.73 | O |
| ATOM | 5942 | N | ASP | B | 269 | −29.779 | 1.930 | 15.494 | 1.00 | 55.18 | N |
| ATOM | 5943 | CA | ASP | B | 269 | −30.687 | 3.028 | 15.501 | 1.00 | 51.61 | C |
| ATOM | 5944 | CB | ASP | B | 269 | −30.051 | 4.244 | 14.828 | 1.00 | 48.20 | C |
| ATOM | 5945 | CG | ASP | B | 269 | −28.959 | 4.864 | 15.657 | 1.00 | 50.61 | C |
| ATOM | 5946 | OD1 | ASP | B | 269 | −28.481 | 4.197 | 16.597 | 1.00 | 52.62 | O |
| ATOM | 5947 | OD2 | ASP | B | 269 | −28.579 | 6.019 | 15.366 | 1.00 | 52.51 | O |
| ATOM | 5948 | C | ASP | B | 269 | −31.878 | 2.635 | 14.705 | 1.00 | 52.34 | C |
| ATOM | 5949 | O | ASP | B | 269 | −32.813 | 3.385 | 14.612 | 1.00 | 53.94 | O |
| ATOM | 5950 | N | TYR | B | 270 | −31.873 | 1.394 | 14.229 | 1.00 | 52.55 | N |
| ATOM | 5951 | CA | TYR | B | 270 | −32.997 | 0.802 | 13.554 | 1.00 | 51.93 | C |
| ATOM | 5952 | CB | TYR | B | 270 | −32.622 | −0.469 | 12.784 | 1.00 | 53.16 | C |
| ATOM | 5953 | CG | TYR | B | 270 | −31.960 | −0.208 | 11.446 | 1.00 | 55.70 | C |
| ATOM | 5954 | CD2 | TYR | B | 270 | −32.025 | −1.143 | 10.420 | 1.00 | 51.25 | C |
| ATOM | 5955 | CE2 | TYR | B | 270 | −31.421 | −0.909 | 9.201 | 1.00 | 52.67 | C |
| ATOM | 5956 | CZ | TYR | B | 270 | −30.745 | 0.275 | 8.996 | 1.00 | 56.78 | C |
| ATOM | 5957 | OH | TYR | B | 270 | −30.134 | 0.532 | 7.790 | 1.00 | 57.40 | O |
| ATOM | 5958 | CE1 | TYR | B | 270 | −30.674 | 1.216 | 9.994 | 1.00 | 60.69 | C |
| ATOM | 5959 | CD1 | TYR | B | 270 | −31.281 | 0.977 | 11.205 | 1.00 | 58.09 | C |
| ATOM | 5960 | C | TYR | B | 270 | −34.081 | 0.547 | 14.550 | 1.00 | 54.86 | C |
| ATOM | 5961 | O | TYR | B | 270 | −35.251 | 0.561 | 14.230 | 1.00 | 54.76 | O |
| ATOM | 5962 | N | ILE | B | 271 | −33.679 | 0.374 | 15.799 | 1.00 | 48.40 | N |
| ATOM | 5963 | CA | ILE | B | 271 | −34.623 | 0.142 | 16.851 | 1.00 | 49.29 | C |
| ATOM | 5964 | CB | ILE | B | 271 | −33.909 | 0.038 | 18.213 | 1.00 | 43.97 | C |
| ATOM | 5965 | CG1 | ILE | B | 271 | −32.881 | −1.089 | 18.189 | 1.00 | 44.01 | C |
| ATOM | 5966 | CD1 | ILE | B | 271 | −32.155 | −1.265 | 19.509 | 1.00 | 51.87 | C |
| ATOM | 5967 | CG2 | ILE | B | 271 | −34.906 | −0.190 | 19.336 | 1.00 | 38.21 | C |
| ATOM | 5968 | C | ILE | B | 271 | −35.684 | 1.251 | 16.936 | 1.00 | 47.68 | C |
| ATOM | 5969 | O | ILE | B | 271 | −36.848 | 0.951 | 17.084 | 1.00 | 38.67 | O |
| ATOM | 5970 | N | TYR | B | 272 | −35.292 | 2.527 | 16.768 | 1.00 | 51.77 | N |
| ATOM | 5971 | CA | TYR | B | 272 | −36.275 | 3.616 | 16.735 | 1.00 | 54.40 | C |
| ATOM | 5972 | CB | TYR | B | 272 | −35.868 | 4.785 | 17.650 | 1.00 | 44.42 | C |
| ATOM | 5973 | CG | TYR | B | 272 | −34.848 | 5.739 | 17.084 | 1.00 | 39.71 | C |
| ATOM | 5974 | CD2 | TYR | B | 272 | −35.243 | 6.828 | 16.322 | 1.00 | 43.35 | C |
| ATOM | 5975 | CE2 | TYR | B | 272 | −34.324 | 7.711 | 15.811 | 1.00 | 47.01 | C |
| ATOM | 5976 | CZ | TYR | B | 272 | −32.987 | 7.523 | 16.067 | 1.00 | 46.35 | C |
| ATOM | 5977 | OH | TYR | B | 272 | −32.076 | 8.413 | 15.551 | 1.00 | 57.65 | O |
| ATOM | 5978 | CE1 | TYR | B | 272 | −32.565 | 6.455 | 16.828 | 1.00 | 41.96 | C |
| ATOM | 5979 | CD1 | TYR | B | 272 | −33.496 | 5.572 | 17.334 | 1.00 | 38.33 | C |
| ATOM | 5980 | C | TYR | B | 272 | −36.688 | 4.073 | 15.324 | 1.00 | 48.09 | C |
| ATOM | 5981 | O | TYR | B | 272 | −37.857 | 4.269 | 15.050 | 1.00 | 47.49 | O |
| ATOM | 5982 | N | GLN | B | 273 | −35.716 | 4.160 | 14.420 | 1.00 | 42.25 | N |
| ATOM | 5983 | CA | GLN | B | 273 | −35.908 | 4.488 | 13.011 | 1.00 | 44.21 | C |
| ATOM | 5984 | CB | GLN | B | 273 | −34.573 | 4.563 | 12.276 | 1.00 | 48.01 | C |
| ATOM | 5985 | CG | GLN | B | 273 | −33.726 | 5.745 | 12.728 | 1.00 | 50.15 | C |
| ATOM | 5986 | CD | GLN | B | 273 | −32.316 | 5.693 | 12.187 | 1.00 | 45.98 | C |
| ATOM | 5987 | OE1 | GLN | B | 273 | −31.948 | 4.759 | 11.478 | 1.00 | 46.63 | O |
| ATOM | 5988 | NE2 | GLN | B | 273 | −31.516 | 6.698 | 12.519 | 1.00 | 47.42 | N |
| ATOM | 5989 | C | GLN | B | 273 | −36.884 | 3.546 | 12.312 | 1.00 | 48.27 | C |
| ATOM | 5990 | O | GLN | B | 273 | −37.745 | 3.955 | 11.558 | 1.00 | 45.31 | O |
| ATOM | 5991 | N | GLY | B | 274 | −36.759 | 2.265 | 12.618 | 1.00 | 48.13 | N |
| ATOM | 5992 | CA | GLY | B | 274 | −37.631 | 1.232 | 12.154 | 1.00 | 43.91 | C |
| ATOM | 5993 | C | GLY | B | 274 | −39.069 | 1.539 | 12.391 | 1.00 | 43.61 | C |
| ATOM | 5994 | O | GLY | B | 274 | −39.755 | 1.673 | 11.408 | 1.00 | 46.13 | O |
| ATOM | 5995 | N | PRO | B | 275 | −39.604 | 1.744 | 13.611 | 1.00 | 45.54 | N |
| ATOM | 5996 | CA | PRO | B | 275 | −40.977 | 2.092 | 13.886 | 1.00 | 37.91 | C |
| ATOM | 5997 | CB | PRO | B | 275 | −40.990 | 2.286 | 15.403 | 1.00 | 35.10 | C |
| ATOM | 5998 | CG | PRO | B | 275 | −39.968 | 1.333 | 15.897 | 1.00 | 36.88 | C |
| ATOM | 5999 | CD | PRO | B | 275 | −38.893 | 1.383 | 14.835 | 1.00 | 45.43 | C |
| ATOM | 6000 | C | PRO | B | 275 | −41.345 | 3.376 | 13.193 | 1.00 | 38.85 | C |
| ATOM | 6001 | O | PRO | B | 275 | −42.466 | 3.519 | 12.784 | 1.00 | 45.79 | O |
| ATOM | 6002 | N | MET | B | 276 | −40.451 | 4.320 | 13.079 | 1.00 | 39.21 | N |
| ATOM | 6003 | CA | MET | B | 276 | −40.782 | 5.546 | 12.421 | 1.00 | 38.24 | C |
| ATOM | 6004 | CB | MET | B | 276 | −39.645 | 6.560 | 12.558 | 1.00 | 42.60 | C |
| ATOM | 6005 | CG | MET | B | 276 | −39.478 | 7.081 | 13.978 | 1.00 | 42.13 | C |
| ATOM | 6006 | SD | MET | B | 276 | −37.958 | 8.015 | 14.226 | 1.00 | 44.94 | S |
| ATOM | 6007 | CE | MET | B | 276 | −38.190 | 9.355 | 13.064 | 1.00 | 40.37 | C |
| ATOM | 6008 | C | MET | B | 276 | −41.142 | 5.310 | 10.961 | 1.00 | 40.17 | C |
| ATOM | 6009 | O | MET | B | 276 | −42.131 | 5.794 | 10.457 | 1.00 | 40.11 | O |
| ATOM | 6010 | N | ALA | B | 277 | −40.345 | 4.493 | 10.303 | 1.00 | 45.53 | N |
| ATOM | 6011 | CA | ALA | B | 277 | −40.602 | 4.105 | 8.932 | 1.00 | 44.51 | C |
| ATOM | 6012 | CB | ALA | B | 277 | −39.440 | 3.285 | 8.386 | 1.00 | 37.30 | C |
| ATOM | 6013 | C | ALA | B | 277 | −41.905 | 3.335 | 8.798 | 1.00 | 44.76 | C |
| ATOM | 6014 | O | ALA | B | 277 | −42.702 | 3.583 | 7.928 | 1.00 | 45.21 | O |
| ATOM | 6015 | N | LEU | B | 278 | −42.112 | 2.399 | 9.699 | 1.00 | 42.06 | N |
| ATOM | 6016 | CA | LEU | B | 278 | −43.292 | 1.569 | 9.728 | 1.00 | 45.31 | C |
| ATOM | 6017 | CB | LEU | B | 278 | −43.214 | 0.589 | 10.898 | 1.00 | 44.48 | C |
| ATOM | 6018 | CG | LEU | B | 278 | −44.480 | −0.227 | 11.151 | 1.00 | 47.97 | C |
| ATOM | 6019 | CD1 | LEU | B | 278 | −44.693 | −1.261 | 10.052 | 1.00 | 42.29 | C |
| ATOM | 6020 | CD2 | LEU | B | 278 | −44.416 | −0.881 | 12.521 | 1.00 | 50.94 | C |
| ATOM | 6021 | C | LEU | B | 278 | −44.561 | 2.381 | 9.815 | 1.00 | 44.60 | C |
| ATOM | 6022 | O | LEU | B | 278 | −45.479 | 2.189 | 9.056 | 1.00 | 45.19 | O |
| ATOM | 6023 | N | VAL | B | 279 | −44.552 | 3.340 | 10.711 | 1.00 | 39.33 | N |
| ATOM | 6024 | CA | VAL | B | 279 | −45.627 | 4.272 | 10.917 | 1.00 | 39.14 | C |
| ATOM | 6025 | CB | VAL | B | 279 | −45.335 | 5.130 | 12.152 | 1.00 | 35.41 | C |
| ATOM | 6026 | CG1 | VAL | B | 279 | −46.058 | 6.451 | 12.083 | 1.00 | 41.10 | C |
| ATOM | 6027 | CG2 | VAL | B | 279 | −45.709 | 4.362 | 13.404 | 1.00 | 40.12 | C |
| ATOM | 6028 | C | VAL | B | 279 | −45.894 | 5.157 | 9.674 | 1.00 | 46.06 | C |
| ATOM | 6029 | O | VAL | B | 279 | −47.029 | 5.387 | 9.285 | 1.00 | 48.82 | O |
| ATOM | 6030 | N | LEU | B | 280 | −44.824 | 5.619 | 9.019 | 1.00 | 43.59 | N |
| ATOM | 6031 | CA | LEU | B | 280 | −44.945 | 6.378 | 7.777 | 1.00 | 42.82 | C |
| ATOM | 6032 | CB | LEU | B | 280 | −43.560 | 6.797 | 7.277 | 1.00 | 39.14 | C |
| ATOM | 6033 | CG | LEU | B | 280 | −43.522 | 7.732 | 6.072 | 1.00 | 32.94 | C |
| ATOM | 6034 | CD1 | LEU | B | 280 | −44.194 | 9.047 | 6.401 | 1.00 | 36.15 | C |
| ATOM | 6035 | CD2 | LEU | B | 280 | −42.096 | 7.970 | 5.643 | 1.00 | 41.32 | C |
| ATOM | 6036 | C | LEU | B | 280 | −45.668 | 5.594 | 6.694 | 1.00 | 44.28 | C |
| ATOM | 6037 | O | LEU | B | 280 | −46.540 | 6.092 | 6.024 | 1.00 | 43.60 | O |
| ATOM | 6038 | N | LEU | B | 281 | −45.295 | 4.335 | 6.550 | 1.00 | 49.64 | N |
| ATOM | 6039 | CA | LEU | B | 281 | −45.909 | 3.403 | 5.608 | 1.00 | 50.71 | C |
| ATOM | 6040 | CB | LEU | B | 281 | −45.214 | 2.045 | 5.718 | 1.00 | 49.55 | C |
| ATOM | 6041 | CG | LEU | B | 281 | −45.557 | 0.960 | 4.702 | 1.00 | 48.59 | C |
| ATOM | 6042 | CD1 | LEU | B | 281 | −44.888 | 1.239 | 3.368 | 1.00 | 48.49 | C |
| ATOM | 6043 | CD2 | LEU | B | 281 | −45.141 | −0.396 | 5.244 | 1.00 | 49.43 | C |
| ATOM | 6044 | C | LEU | B | 281 | −47.405 | 3.240 | 5.804 | 1.00 | 47.70 | C |
| ATOM | 6045 | O | LEU | B | 281 | −48.187 | 3.386 | 4.895 | 1.00 | 51.13 | O |
| ATOM | 6046 | N | ILE | B | 282 | −47.807 | 2.989 | 7.031 | 1.00 | 41.10 | N |
| ATOM | 6047 | CA | ILE | B | 282 | −49.210 | 2.889 | 7.362 | 1.00 | 46.38 | C |
| ATOM | 6048 | CB | ILE | B | 282 | −49.403 | 2.483 | 8.832 | 1.00 | 41.49 | C |
| ATOM | 6049 | CG1 | ILE | B | 282 | −48.688 | 1.159 | 9.100 | 1.00 | 38.56 | C |
| ATOM | 6050 | CD1 | ILE | B | 282 | −48.932 | 0.607 | 10.484 | 1.00 | 41.73 | C |
| ATOM | 6051 | CG2 | ILE | B | 282 | −50.882 | 2.376 | 9.174 | 1.00 | 38.53 | C |
| ATOM | 6052 | C | ILE | B | 282 | −49.969 | 4.210 | 7.076 | 1.00 | 46.41 | C |
| ATOM | 6053 | O | ILE | B | 282 | −51.090 | 4.212 | 6.600 | 1.00 | 49.62 | O |
| ATOM | 6054 | N | ASN | B | 283 | −49.319 | 5.340 | 7.332 | 1.00 | 41.98 | N |
| ATOM | 6055 | CA | ASN | B | 283 | −49.865 | 6.650 | 7.020 | 1.00 | 43.83 | C |
| ATOM | 6056 | CB | ASN | B | 283 | −48.987 | 7.775 | 7.572 | 1.00 | 49.26 | C |
| ATOM | 6057 | CG | ASN | B | 283 | −49.749 | 8.693 | 8.513 | 1.00 | 50.20 | C |
| ATOM | 6058 | OD1 | ASN | B | 283 | −50.797 | 8.322 | 9.043 | 1.00 | 52.74 | O |
| ATOM | 6059 | ND2 | ASN | B | 283 | −49.232 | 9.898 | 8.719 | 1.00 | 44.95 | N |
| ATOM | 6060 | C | ASN | B | 283 | −50.149 | 6.830 | 5.531 | 1.00 | 45.34 | C |
| ATOM | 6061 | O | ASN | B | 283 | −51.176 | 7.336 | 5.147 | 1.00 | 44.76 | O |
| ATOM | 6062 | N | PHE | B | 284 | −49.260 | 6.323 | 4.697 | 1.00 | 51.60 | N |
| ATOM | 6063 | CA | PHE | B | 284 | −49.473 | 6.292 | 3.247 | 1.00 | 51.17 | C |
| ATOM | 6064 | CB | PHE | B | 284 | −48.216 | 5.852 | 2.501 | 1.00 | 43.39 | C |
| ATOM | 6065 | CG | PHE | B | 284 | −47.270 | 6.975 | 2.209 | 1.00 | 52.02 | C |
| ATOM | 6066 | CD1 | PHE | B | 284 | −47.538 | 7.868 | 1.183 | 1.00 | 53.39 | C |
| ATOM | 6067 | CE1 | PHE | B | 284 | −46.672 | 8.909 | 0.904 | 1.00 | 53.82 | C |
| ATOM | 6068 | CZ | PHE | B | 284 | −45.519 | 9.072 | 1.654 | 1.00 | 58.17 | C |
| ATOM | 6069 | CE2 | PHE | B | 284 | −45.238 | 8.191 | 2.683 | 1.00 | 57.23 | C |
| ATOM | 6070 | CD2 | PHE | B | 284 | −46.115 | 7.146 | 2.959 | 1.00 | 56.70 | C |
| ATOM | 6071 | C | PHE | B | 284 | −50.673 | 5.407 | 2.878 | 1.00 | 53.11 | C |
| ATOM | 6072 | O | PHE | B | 284 | −51.488 | 5.773 | 2.061 | 1.00 | 53.07 | O |
| ATOM | 6073 | N | ILE | B | 285 | −50.814 | 4.251 | 3.529 | 1.00 | 51.35 | N |
| ATOM | 6074 | CA | ILE | B | 285 | −51.984 | 3.410 | 3.320 | 1.00 | 48.69 | C |
| ATOM | 6075 | CB | ILE | B | 285 | −51.901 | 2.111 | 4.133 | 1.00 | 50.82 | C |
| ATOM | 6076 | CG1 | ILE | B | 285 | −50.659 | 1.316 | 3.723 | 1.00 | 46.45 | C |
| ATOM | 6077 | CD1 | ILE | B | 285 | −50.511 | −0.008 | 4.442 | 1.00 | 50.00 | C |
| ATOM | 6078 | CG2 | ILE | B | 285 | −53.175 | 1.285 | 3.957 | 1.00 | 46.73 | C |
| ATOM | 6079 | C | ILE | B | 285 | −53.273 | 4.159 | 3.678 | 1.00 | 53.83 | C |
| ATOM | 6080 | O | ILE | B | 285 | −54.206 | 4.199 | 2.894 | 1.00 | 68.68 | O |
| ATOM | 6081 | N | PHE | B | 286 | −53.279 | 4.859 | 4.810 | 1.00 | 50.86 | N |
| ATOM | 6082 | CA | PHE | B | 286 | −54.413 | 5.700 | 5.174 | 1.00 | 54.71 | C |
| ATOM | 6083 | CB | PHE | B | 286 | −54.161 | 6.408 | 6.508 | 1.00 | 55.48 | C |
| ATOM | 6084 | CG | PHE | B | 286 | −54.234 | 5.512 | 7.703 | 1.00 | 53.95 | C |
| ATOM | 6085 | CD1 | PHE | B | 286 | −54.693 | 4.213 | 7.587 | 1.00 | 58.66 | C |
| ATOM | 6086 | CE1 | PHE | B | 286 | −54.758 | 3.389 | 8.693 | 1.00 | 66.21 | C |
| ATOM | 6087 | CZ | PHE | B | 286 | −54.366 | 3.863 | 9.933 | 1.00 | 62.89 | C |
| ATOM | 6088 | CE2 | PHE | B | 286 | −53.910 | 5.160 | 10.061 | 1.00 | 53.63 | C |
| ATOM | 6089 | CD2 | PHE | B | 286 | −53.848 | 5.977 | 8.951 | 1.00 | 50.06 | C |
| ATOM | 6090 | C | PHE | B | 286 | −54.697 | 6.774 | 4.153 | 1.00 | 54.68 | C |
| ATOM | 6091 | O | PHE | B | 286 | −55.823 | 6.978 | 3.791 | 1.00 | 60.55 | O |
| ATOM | 6092 | N | LEU | B | 287 | −53.667 | 7.460 | 3.700 | 1.00 | 51.96 | N |
| ATOM | 6093 | CA | LEU | B | 287 | −53.797 | 8.509 | 2.703 | 1.00 | 49.36 | C |
| ATOM | 6094 | CB | LEU | B | 287 | −52.446 | 9.178 | 2.433 | 1.00 | 47.56 | C |
| ATOM | 6095 | CG | LEU | B | 287 | −52.480 | 10.450 | 1.585 | 1.00 | 38.08 | C |
| ATOM | 6096 | CD1 | LEU | B | 287 | −53.348 | 11.504 | 2.236 | 1.00 | 42.57 | C |
| ATOM | 6097 | CD2 | LEU | B | 287 | −51.082 | 10.990 | 1.370 | 1.00 | 47.03 | C |
| ATOM | 6098 | C | LEU | B | 287 | −54.402 | 7.986 | 1.410 | 1.00 | 54.09 | C |
| ATOM | 6099 | O | LEU | B | 287 | −55.242 | 8.638 | 0.817 | 1.00 | 55.68 | O |
| ATOM | 6100 | N | PHE | B | 288 | −53.959 | 6.792 | 0.953 | 1.00 | 60.60 | N |
| ATOM | 6101 | CA | PHE | B | 288 | −54.534 | 6.187 | −0.249 | 1.00 | 65.84 | C |
| ATOM | 6102 | CB | PHE | B | 288 | −53.895 | 4.808 | −0.472 | 1.00 | 74.85 | C |
| ATOM | 6103 | CG | PHE | B | 288 | −54.542 | 3.983 | −1.554 | 1.00 | 100.11 | C |
| ATOM | 6104 | CD1 | PHE | B | 288 | −54.154 | 4.127 | −2.873 | 1.00 | 100.49 | C |
| ATOM | 6105 | CE1 | PHE | B | 288 | −54.728 | 3.363 | −3.862 | 1.00 | 107.29 | C |
| ATOM | 6106 | CZ | PHE | B | 288 | −55.692 | 2.432 | −3.546 | 1.00 | 117.27 | C |
| ATOM | 6107 | CE2 | PHE | B | 288 | −56.083 | 2.264 | −2.237 | 1.00 | 106.29 | C |
| ATOM | 6108 | CD2 | PHE | B | 288 | −55.505 | 3.030 | −1.244 | 1.00 | 105.72 | C |
| ATOM | 6109 | C | PHE | B | 288 | −56.046 | 6.061 | −0.111 | 1.00 | 69.37 | C |
| ATOM | 6110 | O | PHE | B | 288 | −56.794 | 6.500 | −0.957 | 1.00 | 72.56 | O |
| ATOM | 6111 | N | ASN | B | 289 | −56.493 | 5.481 | 0.993 | 1.00 | 61.80 | N |
| ATOM | 6112 | CA | ASN | B | 289 | −57.920 | 5.357 | 1.247 | 1.00 | 61.55 | C |
| ATOM | 6113 | CB | ASN | B | 289 | −58.159 | 4.468 | 2.463 | 1.00 | 68.44 | C |
| ATOM | 6114 | CG | ASN | B | 289 | −57.736 | 3.040 | 2.213 | 1.00 | 78.22 | C |
| ATOM | 6115 | OD1 | ASN | B | 289 | −57.786 | 2.560 | 1.077 | 1.00 | 78.31 | O |
| ATOM | 6116 | ND2 | ASN | B | 289 | −57.314 | 2.350 | 3.265 | 1.00 | 85.53 | N |
| ATOM | 6117 | C | ASN | B | 289 | −58.674 | 6.679 | 1.387 | 1.00 | 60.29 | C |
| ATOM | 6118 | O | ASN | B | 289 | −59.755 | 6.808 | 0.871 | 1.00 | 66.02 | O |
| ATOM | 6119 | N | ILE | B | 290 | −58.138 | 7.649 | 2.121 | 1.00 | 58.78 | N |
| ATOM | 6120 | CA | ILE | B | 290 | −58.838 | 8.920 | 2.314 | 1.00 | 54.07 | C |
| ATOM | 6121 | CB | ILE | B | 290 | −58.092 | 9.836 | 3.293 | 1.00 | 56.18 | C |
| ATOM | 6122 | CG1 | ILE | B | 290 | −57.985 | 9.162 | 4.661 | 1.00 | 59.17 | C |
| ATOM | 6123 | CD1 | ILE | B | 290 | −57.215 | 9.970 | 5.677 | 1.00 | 51.65 | C |
| ATOM | 6124 | CG2 | ILE | B | 290 | −58.802 | 11.172 | 3.421 | 1.00 | 49.73 | C |
| ATOM | 6125 | C | ILE | B | 290 | −59.052 | 9.640 | 1.007 | 1.00 | 54.80 | C |
| ATOM | 6126 | O | ILE | B | 290 | −60.140 | 10.100 | 0.719 | 1.00 | 55.09 | O |
| ATOM | 6127 | N | VAL | B | 291 | −58.027 | 9.622 | 0.162 | 1.00 | 58.57 | N |
| ATOM | 6128 | CA | VAL | B | 291 | −58.136 | 10.114 | −1.200 | 1.00 | 59.14 | C |
| ATOM | 6129 | CB | VAL | B | 291 | −56.777 | 10.099 | −1.938 | 1.00 | 53.27 | C |
| ATOM | 6130 | CG1 | VAL | B | 291 | −56.937 | 10.608 | −3.363 | 1.00 | 50.43 | C |
| ATOM | 6131 | CG2 | VAL | B | 291 | −55.766 | 10.951 | −1.199 | 1.00 | 52.67 | C |
| ATOM | 6132 | C | VAL | B | 291 | −59.166 | 9.323 | −2.001 | 1.00 | 61.00 | C |
| ATOM | 6133 | O | VAL | B | 291 | −59.976 | 9.897 | −2.697 | 1.00 | 64.01 | O |
| ATOM | 6134 | N | ARG | B | 292 | −59.154 | 7.990 | −1.878 | 1.00 | 57.90 | N |
| ATOM | 6135 | CA | ARG | B | 292 | −60.118 | 7.158 | −2.591 | 1.00 | 59.58 | C |
| ATOM | 6136 | CB | ARG | B | 292 | −59.877 | 5.670 | −2.305 | 1.00 | 66.38 | C |
| ATOM | 6137 | CG | ARG | B | 292 | −60.848 | 4.734 | −3.022 | 1.00 | 66.03 | C |
| ATOM | 6138 | CD | ARG | B | 292 | −60.592 | 3.270 | −2.678 | 1.00 | 72.25 | C |
| ATOM | 6139 | NE | ARG | B | 292 | −60.777 | 2.994 | −1.255 | 1.00 | 82.33 | N |
| ATOM | 6140 | CZ | ARG | B | 292 | −61.940 | 2.662 | −0.699 | 1.00 | 92.02 | C |
| ATOM | 6141 | NH1 | ARG | B | 292 | −63.035 | 2.568 | −1.446 | 1.00 | 86.56 | N |
| ATOM | 6142 | NH2 | ARG | B | 292 | −62.012 | 2.425 | 0.605 | 1.00 | 89.27 | N |
| ATOM | 6143 | C | ARG | B | 292 | −61.528 | 7.514 | −2.229 | 1.00 | 58.15 | C |
| ATOM | 6144 | O | ARG | B | 292 | −62.358 | 7.628 | −3.086 | 1.00 | 63.51 | O |
| ATOM | 6145 | N | ILE | B | 293 | −61.796 | 7.662 | −0.952 | 1.00 | 54.24 | N |
| ATOM | 6146 | CA | ILE | B | 293 | −63.127 | 7.969 | −0.485 | 1.00 | 56.29 | C |
| ATOM | 6147 | CB | ILE | B | 293 | −63.233 | 7.845 | 1.045 | 1.00 | 52.60 | C |
| ATOM | 6148 | CG1 | ILE | B | 293 | −62.933 | 6.406 | 1.472 | 1.00 | 54.91 | C |
| ATOM | 6149 | CD1 | ILE | B | 293 | −63.160 | 6.136 | 2.943 | 1.00 | 53.68 | C |
| ATOM | 6150 | CG2 | ILE | B | 293 | −64.615 | 8.261 | 1.518 | 1.00 | 48.99 | C |
| ATOM | 6151 | C | ILE | B | 293 | −63.578 | 9.352 | −0.940 | 1.00 | 60.35 | C |
| ATOM | 6152 | O | ILE | B | 293 | −64.653 | 9.541 | −1.468 | 1.00 | 62.35 | O |
| ATOM | 6153 | N | LEU | B | 294 | −62.712 | 10.321 | −0.816 | 1.00 | 61.83 | N |
| ATOM | 6154 | CA | LEU | B | 294 | −63.032 | 11.653 | −1.273 | 1.00 | 64.57 | C |
| ATOM | 6155 | CB | LEU | B | 294 | −61.891 | 12.617 | −0.951 | 1.00 | 62.71 | C |
| ATOM | 6156 | CG | LEU | B | 294 | −61.744 | 12.889 | 0.545 | 1.00 | 57.00 | C |
| ATOM | 6157 | CD1 | LEU | B | 294 | −60.508 | 13.716 | 0.814 | 1.00 | 58.15 | C |
| ATOM | 6158 | CD2 | LEU | B | 294 | −62.989 | 13.579 | 1.087 | 1.00 | 54.29 | C |
| ATOM | 6159 | C | LEU | B | 294 | −63.406 | 11.720 | −2.747 | 1.00 | 63.75 | C |
| ATOM | 6160 | O | LEU | B | 294 | −64.422 | 12.270 | −3.114 | 1.00 | 72.30 | O |
| ATOM | 6161 | N | MET | B | 295 | −62.569 | 11.154 | −3.584 | 1.00 | 60.87 | N |
| ATOM | 6162 | CA | MET | B | 295 | −62.781 | 11.146 | −5.017 | 1.00 | 69.47 | C |
| ATOM | 6163 | CB | MET | B | 295 | −61.523 | 10.660 | −5.734 | 1.00 | 67.20 | C |
| ATOM | 6164 | CG | MET | B | 295 | −60.289 | 11.464 | −5.375 | 1.00 | 69.26 | C |
| ATOM | 6165 | SD | MET | B | 295 | −59.271 | 11.870 | −6.798 | 1.00 | 93.12 | S |
| ATOM | 6166 | CE | MET | B | 295 | −60.015 | 13.425 | −7.280 | 1.00 | 66.99 | C |
| ATOM | 6167 | C | MET | B | 295 | −64.003 | 10.331 | −5.448 | 1.00 | 73.78 | C |
| ATOM | 6168 | O | MET | B | 295 | −64.772 | 10.770 | −6.280 | 1.00 | 74.83 | O |
| ATOM | 6169 | N | THR | B | 296 | −64.203 | 9.162 | −4.838 | 1.00 | 69.76 | N |
| ATOM | 6170 | CA | THR | B | 296 | −65.363 | 8.354 | −5.125 | 1.00 | 60.97 | C |
| ATOM | 6171 | CB | THR | B | 296 | −64.980 | 6.847 | −5.074 | 1.00 | 61.14 | C |
| ATOM | 6172 | OG1 | THR | B | 296 | −64.839 | 6.423 | −3.711 | 1.00 | 66.48 | O |
| ATOM | 6173 | CG2 | THR | B | 296 | −63.682 | 6.593 | −5.831 | 1.00 | 54.08 | C |
| ATOM | 6174 | C | THR | B | 296 | −66.782 | 8.436 | −4.521 | 1.00 | 64.76 | C |
| ATOM | 6175 | O | THR | B | 296 | −67.764 | 8.502 | −5.248 | 1.00 | 82.27 | O |
| ATOM | 6176 | N | LYS | B | 297 | −66.874 | 8.412 | −3.178 | 1.00 | 64.20 | N |
| ATOM | 6177 | CA | LYS | B | 297 | −68.132 | 8.250 | −2.435 | 1.00 | 68.04 | C |
| ATOM | 6178 | CB | LYS | B | 297 | −67.958 | 7.357 | −1.209 | 1.00 | 69.49 | C |
| ATOM | 6179 | CG | LYS | B | 297 | −67.769 | 5.883 | −1.515 | 1.00 | 82.19 | C |
| ATOM | 6180 | CD | LYS | B | 297 | −67.729 | 5.069 | −0.231 | 1.00 | 89.18 | C |
| ATOM | 6181 | CE | LYS | B | 297 | −67.603 | 3.579 | −0.508 | 1.00 | 100.81 | C |
| ATOM | 6182 | NZ | LYS | B | 297 | −67.538 | 2.794 | 0.758 | 1.00 | 103.59 | N |
| ATOM | 6183 | C | LYS | B | 297 | −68.474 | 9.660 | −1.995 | 1.00 | 71.07 | C |
| ATOM | 6184 | O | LYS | B | 297 | −69.617 | 10.066 | −2.051 | 1.00 | 79.22 | O |
| ATOM | 6185 | N | LEU | B | 298 | −67.494 | 10.368 | −1.455 | 1.00 | 69.73 | N |
| ATOM | 6186 | CA | LEU | B | 298 | −67.734 | 11.672 | −0.873 | 1.00 | 67.07 | C |
| ATOM | 6187 | CB | LEU | B | 298 | −66.962 | 11.835 | 0.439 | 1.00 | 63.53 | C |
| ATOM | 6188 | CG | LEU | B | 298 | −67.323 | 10.880 | 1.577 | 1.00 | 56.46 | C |
| ATOM | 6189 | CD1 | LEU | B | 298 | −66.708 | 11.358 | 2.881 | 1.00 | 55.18 | C |
| ATOM | 6190 | CD2 | LEU | B | 298 | −68.829 | 10.730 | 1.712 | 1.00 | 55.85 | C |
| ATOM | 6191 | C | LEU | B | 298 | −67.403 | 12.819 | −1.825 | 1.00 | 72.56 | C |
| ATOM | 6192 | O | LEU | B | 298 | −67.100 | 13.919 | −1.384 | 1.00 | 75.45 | O |
| ATOM | 6193 | N | ARG | B | 299 | −67.441 | 12.576 | −3.142 | 1.00 | 74.63 | N |
| ATOM | 6194 | CA | ARG | B | 299 | −67.067 | 13.630 | −4.069 | 1.00 | 75.68 | C |
| ATOM | 6195 | CB | ARG | B | 299 | −66.805 | 13.101 | −5.482 | 1.00 | 77.86 | C |
| ATOM | 6196 | CG | ARG | B | 299 | −65.702 | 13.882 | −6.203 | 1.00 | 85.74 | C |
| ATOM | 6197 | CD | ARG | B | 299 | −65.722 | 13.688 | −7.710 | 1.00 | 83.49 | C |
| ATOM | 6198 | NE | ARG | B | 299 | −66.776 | 14.480 | −8.342 | 1.00 | 97.37 | N |
| ATOM | 6199 | CZ | ARG | B | 299 | −66.846 | 14.722 | −9.648 | 1.00 | 98.27 | C |
| ATOM | 6200 | NH1 | ARG | B | 299 | −65.919 | 14.231 | −10.456 | 1.00 | 89.17 | N |
| ATOM | 6201 | NH2 | ARG | B | 299 | −67.834 | 15.458 | −10.149 | 1.00 | 99.30 | N |
| ATOM | 6202 | C | ARG | B | 299 | −68.109 | 14.712 | −4.101 | 1.00 | 76.11 | C |
| ATOM | 6203 | O | ARG | B | 299 | −67.798 | 15.888 | −4.101 | 1.00 | 78.80 | O |
| ATOM | 6204 | N | ALA | B | 300 | −69.353 | 14.301 | −4.177 | 1.00 | 81.40 | N |
| ATOM | 6205 | CA | ALA | B | 300 | −70.417 | 15.245 | −4.239 | 1.00 | 82.14 | C |
| ATOM | 6206 | CB | ALA | B | 300 | −71.524 | 14.807 | −5.141 | 1.00 | 93.23 | C |
| ATOM | 6207 | C | ALA | B | 300 | −70.912 | 15.660 | −2.857 | 1.00 | 82.48 | C |
| ATOM | 6208 | O | ALA | B | 300 | −71.744 | 16.544 | −2.767 | 1.00 | 87.80 | O |
| ATOM | 6209 | N | SER | B | 301 | −70.444 | 15.043 | −1.761 | 1.00 | 78.61 | N |
| ATOM | 6210 | CA | SER | B | 301 | −71.013 | 15.446 | −0.470 | 1.00 | 79.22 | C |
| ATOM | 6211 | CB | SER | B | 301 | −70.821 | 14.347 | 0.572 | 1.00 | 69.37 | C |
| ATOM | 6212 | OG | SER | B | 301 | −69.450 | 14.185 | 0.868 | 1.00 | 71.23 | O |
| ATOM | 6213 | C | SER | B | 301 | −70.372 | 16.710 | 0.021 | 1.00 | 75.66 | C |
| ATOM | 6214 | O | SER | B | 301 | −69.172 | 16.857 | −0.068 | 1.00 | 77.53 | O |
| ATOM | 6215 | N | THR | B | 302 | −71.164 | 17.567 | 0.638 | 1.00 | 80.43 | N |
| ATOM | 6216 | CA | THR | B | 302 | −70.644 | 18.809 | 1.144 | 1.00 | 76.78 | C |
| ATOM | 6217 | CB | THR | B | 302 | −71.050 | 19.996 | 0.253 | 1.00 | 70.38 | C |
| ATOM | 6218 | OG1 | THR | B | 302 | −72.436 | 19.882 | −0.091 | 1.00 | 67.29 | O |
| ATOM | 6219 | CG2 | THR | B | 302 | −70.225 | 20.010 | −1.025 | 1.00 | 72.32 | C |
| ATOM | 6220 | C | THR | B | 302 | −71.189 | 19.000 | 2.532 | 1.00 | 80.53 | C |
| ATOM | 6221 | O | THR | B | 302 | −72.350 | 19.340 | 2.708 | 1.00 | 84.23 | O |
| ATOM | 6222 | O | THR | B | 303 | −68.354 | 19.316 | 5.041 | 1.00 | 87.77 | O |
| ATOM | 6223 | N | THR | B | 303 | −70.338 | 18.717 | 3.508 | 1.00 | 79.23 | N |
| ATOM | 6224 | CA | THR | B | 303 | −70.679 | 18.839 | 4.906 | 1.00 | 82.67 | C |
| ATOM | 6225 | C | THR | B | 303 | −69.411 | 19.201 | 5.651 | 1.00 | 84.68 | C |
| ATOM | 6226 | CB | THR | B | 303 | −71.186 | 17.503 | 5.496 | 1.00 | 82.51 | C |
| ATOM | 6227 | OG1 | THR | B | 303 | −70.281 | 16.447 | 5.145 | 1.00 | 77.01 | O |
| ATOM | 6228 | CG2 | THR | B | 303 | −72.582 | 17.165 | 4.988 | 1.00 | 83.21 | C |
| ATOM | 6229 | O | SER | B | 304 | −66.160 | 19.458 | 7.625 | 1.00 | 78.26 | O |
| ATOM | 6230 | N | SER | B | 304 | −69.551 | 19.532 | 6.939 | 1.00 | 87.19 | N |
| ATOM | 6231 | CA | SER | B | 304 | −68.447 | 20.062 | 7.710 | 1.00 | 79.53 | C |
| ATOM | 6232 | C | SER | B | 304 | −67.304 | 19.075 | 7.780 | 1.00 | 73.90 | C |
| ATOM | 6233 | CB | SER | B | 304 | −68.909 | 20.428 | 9.123 | 1.00 | 88.35 | C |
| ATOM | 6234 | OG | SER | B | 304 | −69.541 | 19.328 | 9.756 | 1.00 | 89.94 | O |
| ATOM | 6235 | N | GLU | B | 305 | −67.629 | 17.802 | 7.958 | 1.00 | 73.68 | N |
| ATOM | 6236 | CA | GLU | B | 305 | −66.616 | 16.755 | 7.973 | 1.00 | 76.92 | C |
| ATOM | 6237 | CB | GLU | B | 305 | −67.247 | 15.419 | 8.383 | 1.00 | 78.80 | C |
| ATOM | 6238 | CG | GLU | B | 305 | −66.251 | 14.385 | 8.887 | 1.00 | 80.30 | C |
| ATOM | 6239 | CD | GLU | B | 305 | −65.568 | 14.818 | 10.173 | 1.00 | 87.96 | C |
| ATOM | 6240 | OE1 | GLU | B | 305 | −66.247 | 14.873 | 11.221 | 1.00 | 85.16 | O |
| ATOM | 6241 | OE2 | GLU | B | 305 | −64.352 | 15.108 | 10.134 | 1.00 | 91.38 | O |
| ATOM | 6242 | C | GLU | B | 305 | −65.824 | 16.587 | 6.653 | 1.00 | 72.67 | C |
| ATOM | 6243 | O | GLU | B | 305 | −64.615 | 16.424 | 6.653 | 1.00 | 69.46 | O |
| ATOM | 6244 | N | THR | B | 306 | −66.524 | 16.591 | 5.525 | 1.00 | 70.70 | N |
| ATOM | 6245 | CA | THR | B | 306 | −65.885 | 16.428 | 4.226 | 1.00 | 69.42 | C |
| ATOM | 6246 | CB | THR | B | 306 | −66.906 | 16.177 | 3.093 | 1.00 | 77.06 | C |
| ATOM | 6247 | OG1 | THR | B | 306 | −67.831 | 17.269 | 3.021 | 1.00 | 89.36 | O |
| ATOM | 6248 | CG2 | THR | B | 306 | −67.669 | 14.891 | 3.347 | 1.00 | 72.86 | C |
| ATOM | 6249 | C | THR | B | 306 | −64.981 | 17.590 | 3.860 | 1.00 | 66.25 | C |
| ATOM | 6250 | O | THR | B | 306 | −63.893 | 17.408 | 3.366 | 1.00 | 65.85 | O |
| ATOM | 6251 | N | ILE | B | 307 | −65.453 | 18.797 | 4.128 | 1.00 | 69.00 | N |
| ATOM | 6252 | CA | ILE | B | 307 | −64.700 | 20.017 | 3.895 | 1.00 | 65.95 | C |
| ATOM | 6253 | CB | ILE | B | 307 | −65.538 | 21.260 | 4.221 | 1.00 | 61.15 | C |
| ATOM | 6254 | CG1 | ILE | B | 307 | −66.732 | 21.336 | 3.272 | 1.00 | 64.42 | C |
| ATOM | 6255 | CD1 | ILE | B | 307 | −67.685 | 22.466 | 3.583 | 1.00 | 81.53 | C |
| ATOM | 6256 | CG2 | ILE | B | 307 | −64.699 | 22.522 | 4.101 | 1.00 | 69.31 | C |
| ATOM | 6257 | C | ILE | B | 307 | −63.372 | 20.041 | 4.678 | 1.00 | 68.79 | C |
| ATOM | 6258 | O | ILE | B | 307 | −62.315 | 20.288 | 4.119 | 1.00 | 69.12 | O |
| ATOM | 6259 | N | GLN | B | 308 | −63.430 | 19.695 | 5.965 | 1.00 | 69.24 | N |
| ATOM | 6260 | CA | GLN | B | 308 | −62.232 | 19.602 | 6.779 | 1.00 | 64.68 | C |
| ATOM | 6261 | CB | GLN | B | 308 | −62.602 | 19.377 | 8.247 | 1.00 | 65.28 | C |
| ATOM | 6262 | CG | GLN | B | 308 | −63.368 | 20.525 | 8.880 | 1.00 | 72.85 | C |
| ATOM | 6263 | CD | GLN | B | 308 | −63.885 | 20.181 | 10.265 | 1.00 | 86.98 | C |
| ATOM | 6264 | OE1 | GLN | B | 308 | −63.332 | 19.319 | 10.951 | 1.00 | 78.09 | O |
| ATOM | 6265 | NE2 | GLN | B | 308 | −64.958 | 20.849 | 10.680 | 1.00 | 97.72 | N |
| ATOM | 6266 | C | GLN | B | 308 | −61.288 | 18.482 | 6.309 | 1.00 | 62.16 | C |
| ATOM | 6267 | O | GLN | B | 308 | −60.081 | 18.656 | 6.276 | 1.00 | 69.30 | O |
| ATOM | 6268 | N | ALA | B | 309 | −61.847 | 17.348 | 5.889 | 1.00 | 62.15 | N |
| ATOM | 6269 | CA | ALA | B | 309 | −61.076 | 16.298 | 5.223 | 1.00 | 60.87 | C |
| ATOM | 6270 | CB | ALA | B | 309 | −61.963 | 15.090 | 4.933 | 1.00 | 63.73 | C |
| ATOM | 6271 | C | ALA | B | 309 | −60.365 | 16.752 | 3.951 | 1.00 | 58.44 | C |
| ATOM | 6272 | O | ALA | B | 309 | −59.247 | 16.378 | 3.691 | 1.00 | 61.10 | O |
| ATOM | 6273 | N | ARG | B | 310 | −61.043 | 17.533 | 3.123 | 1.00 | 62.63 | N |
| ATOM | 6274 | CA | ARG | B | 310 | −60.452 | 18.052 | 1.897 | 1.00 | 60.98 | C |
| ATOM | 6275 | CB | ARG | B | 310 | −61.493 | 18.840 | 1.088 | 1.00 | 62.91 | C |
| ATOM | 6276 | CG | ARG | B | 310 | −62.671 | 18.004 | 0.560 | 1.00 | 62.60 | C |
| ATOM | 6277 | CD | ARG | B | 310 | −63.499 | 18.766 | −0.514 | 1.00 | 60.14 | C |
| ATOM | 6278 | NE | ARG | B | 310 | −64.555 | 17.930 | −1.104 | 1.00 | 59.97 | N |
| ATOM | 6279 | CZ | ARG | B | 310 | −65.827 | 17.927 | −0.701 | 1.00 | 67.40 | C |
| ATOM | 6280 | NH1 | ARG | B | 310 | −66.208 | 18.732 | 0.281 | 1.00 | 67.83 | N |
| ATOM | 6281 | NH2 | ARG | B | 310 | −66.725 | 17.128 | −1.273 | 1.00 | 75.82 | N |
| ATOM | 6282 | C | ARG | B | 310 | −59.234 | 18.898 | 2.194 | 1.00 | 60.46 | C |
| ATOM | 6283 | O | ARG | B | 310 | −58.179 | 18.730 | 1.609 | 1.00 | 57.64 | O |
| ATOM | 6284 | N | LYS | B | 311 | −59.389 | 19.791 | 3.159 | 1.00 | 58.42 | N |
| ATOM | 6285 | CA | LYS | B | 311 | −58.294 | 20.635 | 3.591 | 1.00 | 57.59 | C |
| ATOM | 6286 | CB | LYS | B | 311 | −58.759 | 21.467 | 4.785 | 1.00 | 54.90 | C |
| ATOM | 6287 | CG | LYS | B | 311 | −59.177 | 22.894 | 4.507 | 1.00 | 56.33 | C |
| ATOM | 6288 | CD | LYS | B | 311 | −59.537 | 23.557 | 5.831 | 1.00 | 64.51 | C |
| ATOM | 6289 | CE | LYS | B | 311 | −59.484 | 25.069 | 5.763 | 1.00 | 69.65 | C |
| ATOM | 6290 | NZ | LYS | B | 311 | −59.733 | 25.660 | 7.107 | 1.00 | 66.76 | N |
| ATOM | 6291 | C | LYS | B | 311 | −57.095 | 19.811 | 4.067 | 1.00 | 59.75 | C |
| ATOM | 6292 | O | LYS | B | 311 | −55.964 | 20.075 | 3.714 | 1.00 | 55.08 | O |
| ATOM | 6293 | N | ALA | B | 312 | −57.365 | 18.822 | 4.905 | 1.00 | 55.71 | N |
| ATOM | 6294 | CA | ALA | B | 312 | −56.316 | 18.019 | 5.478 | 1.00 | 49.12 | C |
| ATOM | 6295 | CB | ALA | B | 312 | −56.899 | 17.054 | 6.496 | 1.00 | 48.85 | C |
| ATOM | 6296 | C | ALA | B | 312 | −55.532 | 17.268 | 4.432 | 1.00 | 57.44 | C |
| ATOM | 6297 | O | ALA | B | 312 | −54.321 | 17.259 | 4.453 | 1.00 | 59.81 | O |
| ATOM | 6298 | N | VAL | B | 313 | −56.229 | 16.670 | 3.481 | 1.00 | 57.34 | N |
| ATOM | 6299 | CA | VAL | B | 313 | −55.572 | 15.985 | 2.389 | 1.00 | 59.21 | C |
| ATOM | 6300 | CB | VAL | B | 313 | −56.600 | 15.297 | 1.463 | 1.00 | 52.92 | C |
| ATOM | 6301 | CG1 | VAL | B | 313 | −55.941 | 14.805 | 0.180 | 1.00 | 49.96 | C |
| ATOM | 6302 | CG2 | VAL | B | 313 | −57.259 | 14.152 | 2.192 | 1.00 | 53.08 | C |
| ATOM | 6303 | C | VAL | B | 313 | −54.685 | 16.924 | 1.567 | 1.00 | 58.39 | C |
| ATOM | 6304 | O | VAL | B | 313 | −53.556 | 16.608 | 1.252 | 1.00 | 52.95 | O |
| ATOM | 6305 | N | LYS | B | 314 | −55.221 | 18.084 | 1.203 | 1.00 | 56.94 | N |
| ATOM | 6306 | CA | LYS | B | 314 | −54.497 | 19.012 | 0.347 | 1.00 | 57.26 | C |
| ATOM | 6307 | CB | LYS | B | 314 | −55.374 | 20.211 | −0.021 | 1.00 | 56.87 | C |
| ATOM | 6308 | CG | LYS | B | 314 | −56.542 | 19.887 | −0.930 | 1.00 | 54.24 | C |
| ATOM | 6309 | CD | LYS | B | 314 | −57.248 | 21.157 | −1.365 | 1.00 | 57.92 | C |
| ATOM | 6310 | CE | LYS | B | 314 | −58.520 | 20.850 | −2.135 | 1.00 | 73.87 | C |
| ATOM | 6311 | NZ | LYS | B | 314 | −59.305 | 22.086 | −2.406 | 1.00 | 88.19 | N |
| ATOM | 6312 | C | LYS | B | 314 | −53.245 | 19.503 | 1.013 | 1.00 | 56.11 | C |
| ATOM | 6313 | O | LYS | B | 314 | −52.200 | 19.553 | 0.405 | 1.00 | 57.08 | O |
| ATOM | 6314 | N | ALA | B | 315 | −53.375 | 19.814 | 2.295 | 1.00 | 59.88 | N |
| ATOM | 6315 | CA | ALA | B | 315 | −52.277 | 20.263 | 3.126 | 1.00 | 55.61 | C |
| ATOM | 6316 | CB | ALA | B | 315 | −52.799 | 20.735 | 4.475 | 1.00 | 54.12 | C |
| ATOM | 6317 | C | ALA | B | 315 | −51.216 | 19.189 | 3.310 | 1.00 | 50.86 | C |
| ATOM | 6318 | O | ALA | B | 315 | −50.048 | 19.458 | 3.231 | 1.00 | 51.06 | O |
| ATOM | 6319 | N | THR | B | 316 | −51.631 | 17.954 | 3.529 | 1.00 | 50.95 | N |
| ATOM | 6320 | CA | THR | B | 316 | −50.693 | 16.859 | 3.685 | 1.00 | 51.00 | C |
| ATOM | 6321 | CB | THR | B | 316 | −51.431 | 15.589 | 4.126 | 1.00 | 47.91 | C |
| ATOM | 6322 | OG1 | THR | B | 316 | −52.096 | 15.828 | 5.371 | 1.00 | 49.73 | O |
| ATOM | 6323 | CG2 | THR | B | 316 | −50.460 | 14.434 | 4.282 | 1.00 | 48.47 | C |
| ATOM | 6324 | C | THR | B | 316 | −49.961 | 16.541 | 2.376 | 1.00 | 54.24 | C |
| ATOM | 6325 | O | THR | B | 316 | −48.782 | 16.248 | 2.377 | 1.00 | 53.76 | O |
| ATOM | 6326 | N | LEU | B | 317 | −50.656 | 16.631 | 1.247 | 1.00 | 56.38 | N |
| ATOM | 6327 | CA | LEU | B | 317 | −50.004 | 16.531 | −0.065 | 1.00 | 53.76 | C |
| ATOM | 6328 | CB | LEU | B | 317 | −51.037 | 16.551 | −1.194 | 1.00 | 57.80 | C |
| ATOM | 6329 | CG | LEU | B | 317 | −51.925 | 15.302 | −1.218 | 1.00 | 56.37 | C |
| ATOM | 6330 | CD1 | LEU | B | 317 | −52.960 | 15.385 | −2.325 | 1.00 | 57.51 | C |
| ATOM | 6331 | CD2 | LEU | B | 317 | −51.080 | 14.040 | −1.353 | 1.00 | 43.13 | C |
| ATOM | 6332 | C | LEU | B | 317 | −48.915 | 17.593 | −0.281 | 1.00 | 51.48 | C |
| ATOM | 6333 | O | LEU | B | 317 | −47.818 | 17.244 | −0.619 | 1.00 | 57.32 | O |
| ATOM | 6334 | N | VAL | B | 318 | −49.178 | 18.866 | 0.032 | 1.00 | 53.27 | N |
| ATOM | 6335 | CA | VAL | B | 318 | −48.111 | 19.888 | 0.081 | 1.00 | 51.37 | C |
| ATOM | 6336 | CB | VAL | B | 318 | −48.662 | 21.300 | 0.381 | 1.00 | 50.09 | C |
| ATOM | 6337 | CG1 | VAL | B | 318 | −47.525 | 22.315 | 0.432 | 1.00 | 36.73 | C |
| ATOM | 6338 | CG2 | VAL | B | 318 | −49.693 | 21.706 | −0.657 | 1.00 | 55.98 | C |
| ATOM | 6339 | C | VAL | B | 318 | −46.992 | 19.579 | 1.098 | 1.00 | 50.98 | C |
| ATOM | 6340 | O | VAL | B | 318 | −45.827 | 19.694 | 0.787 | 1.00 | 58.66 | O |
| ATOM | 6341 | N | LEU | B | 319 | −47.364 | 19.338 | 2.358 | 1.00 | 44.66 | N |
| ATOM | 6342 | CA | LEU | B | 319 | −46.413 | 19.233 | 3.462 | 1.00 | 44.71 | C |
| ATOM | 6343 | CB | LEU | B | 319 | −47.156 | 19.374 | 4.796 | 1.00 | 41.08 | C |
| ATOM | 6344 | CG | LEU | B | 319 | −46.351 | 19.130 | 6.079 | 1.00 | 40.49 | C |
| ATOM | 6345 | CD1 | LEU | B | 319 | −45.534 | 20.361 | 6.459 | 1.00 | 38.09 | C |
| ATOM | 6346 | CD2 | LEU | B | 319 | −47.249 | 18.688 | 7.227 | 1.00 | 41.79 | C |
| ATOM | 6347 | C | LEU | B | 319 | −45.587 | 17.984 | 3.520 | 1.00 | 49.04 | C |
| ATOM | 6348 | O | LEU | B | 319 | −44.449 | 18.018 | 3.943 | 1.00 | 51.48 | O |
| ATOM | 6349 | N | LEU | B | 320 | −46.137 | 16.912 | 2.948 | 1.00 | 51.65 | N |
| ATOM | 6350 | CA | LEU | B | 320 | −45.441 | 15.653 | 2.771 | 1.00 | 50.80 | C |
| ATOM | 6351 | CB | LEU | B | 320 | −46.341 | 14.581 | 2.146 | 1.00 | 44.92 | C |
| ATOM | 6352 | CG | LEU | B | 320 | −45.850 | 13.137 | 2.185 | 1.00 | 51.82 | C |
| ATOM | 6353 | CD1 | LEU | B | 320 | −45.111 | 12.853 | 3.480 | 1.00 | 44.99 | C |
| ATOM | 6354 | CD2 | LEU | B | 320 | −47.041 | 12.211 | 2.041 | 1.00 | 56.23 | C |
| ATOM | 6355 | C | LEU | B | 320 | −44.132 | 15.815 | 1.999 | 1.00 | 54.73 | C |
| ATOM | 6356 | O | LEU | B | 320 | −43.110 | 15.452 | 2.528 | 1.00 | 56.87 | O |
| ATOM | 6357 | N | PRO | B | 321 | −44.021 | 16.331 | 0.776 | 1.00 | 52.84 | N |
| ATOM | 6358 | CA | PRO | B | 321 | −42.734 | 16.475 | 0.119 | 1.00 | 57.45 | C |
| ATOM | 6359 | CB | PRO | B | 321 | −43.104 | 16.997 | −1.276 | 1.00 | 56.51 | C |
| ATOM | 6360 | CG | PRO | B | 321 | −44.505 | 16.605 | −1.465 | 1.00 | 54.42 | C |
| ATOM | 6361 | CD | PRO | B | 321 | −45.101 | 16.758 | −0.112 | 1.00 | 46.37 | C |
| ATOM | 6362 | C | PRO | B | 321 | −41.785 | 17.453 | 0.790 | 1.00 | 58.02 | C |
| ATOM | 6363 | O | PRO | B | 321 | −40.590 | 17.229 | 0.748 | 1.00 | 63.82 | O |
| ATOM | 6364 | N | LEU | B | 322 | −42.280 | 18.486 | 1.441 | 1.00 | 55.77 | N |
| ATOM | 6365 | CA | LEU | B | 322 | −41.409 | 19.412 | 2.137 | 1.00 | 60.80 | C |
| ATOM | 6366 | CB | LEU | B | 322 | −42.212 | 20.561 | 2.754 | 1.00 | 55.93 | C |
| ATOM | 6367 | CG | LEU | B | 322 | −42.585 | 21.683 | 1.780 | 1.00 | 47.69 | C |
| ATOM | 6368 | CD1 | LEU | B | 322 | −43.561 | 22.655 | 2.420 | 1.00 | 48.19 | C |
| ATOM | 6369 | CD2 | LEU | B | 322 | −41.337 | 22.414 | 1.307 | 1.00 | 34.08 | C |
| ATOM | 6370 | C | LEU | B | 322 | −40.538 | 18.735 | 3.186 | 1.00 | 62.22 | C |
| ATOM | 6371 | O | LEU | B | 322 | −39.364 | 19.034 | 3.340 | 1.00 | 62.08 | O |
| ATOM | 6372 | N | LEU | B | 323 | −41.155 | 17.855 | 3.972 | 1.00 | 64.46 | N |
| ATOM | 6373 | CA | LEU | B | 323 | −40.438 | 17.255 | 5.079 | 1.00 | 70.01 | C |
| ATOM | 6374 | CB | LEU | B | 323 | −41.382 | 16.991 | 6.257 | 1.00 | 58.23 | C |
| ATOM | 6375 | CG | LEU | B | 323 | −42.339 | 18.077 | 6.747 | 1.00 | 56.36 | C |
| ATOM | 6376 | CD1 | LEU | B | 323 | −43.160 | 17.549 | 7.918 | 1.00 | 43.14 | C |
| ATOM | 6377 | CD2 | LEU | B | 323 | −41.590 | 19.343 | 7.136 | 1.00 | 61.71 | C |
| ATOM | 6378 | C | LEU | B | 323 | −39.840 | 15.945 | 4.658 | 1.00 | 70.37 | C |
| ATOM | 6379 | O | LEU | B | 323 | −38.880 | 15.492 | 5.245 | 1.00 | 69.89 | O |
| ATOM | 6380 | N | GLY | B | 324 | −40.465 | 15.317 | 3.679 | 1.00 | 65.78 | N |
| ATOM | 6381 | CA | GLY | B | 324 | −40.015 | 14.080 | 3.100 | 1.00 | 62.80 | C |
| ATOM | 6382 | C | GLY | B | 324 | −38.723 | 14.138 | 2.394 | 1.00 | 65.96 | C |
| ATOM | 6383 | O | GLY | B | 324 | −37.937 | 13.209 | 2.429 | 1.00 | 70.66 | O |
| ATOM | 6384 | N | ILE | B | 325 | −38.576 | 15.189 | 1.607 | 1.00 | 66.70 | N |
| ATOM | 6385 | CA | ILE | B | 325 | −37.448 | 15.337 | 0.719 | 1.00 | 70.92 | C |
| ATOM | 6386 | CB | ILE | B | 325 | −37.565 | 16.620 | −0.145 | 1.00 | 69.03 | C |
| ATOM | 6387 | CG1 | ILE | B | 325 | −36.435 | 16.695 | −1.172 | 1.00 | 85.42 | C |
| ATOM | 6388 | CD1 | ILE | B | 325 | −36.541 | 17.888 | −2.106 | 1.00 | 87.07 | C |
| ATOM | 6389 | CG2 | ILE | B | 325 | −37.578 | 17.860 | 0.723 | 1.00 | 69.56 | C |
| ATOM | 6390 | C | ILE | B | 325 | −36.112 | 15.273 | 1.471 | 1.00 | 71.18 | C |
| ATOM | 6391 | O | ILE | B | 325 | −35.168 | 14.643 | 1.028 | 1.00 | 68.92 | O |
| ATOM | 6392 | N | THR | B | 326 | −36.078 | 15.845 | 2.669 | 1.00 | 72.55 | N |
| ATOM | 6393 | CA | THR | B | 326 | −34.906 | 15.780 | 3.511 | 1.00 | 72.99 | C |
| ATOM | 6394 | CB | THR | B | 326 | −35.119 | 16.576 | 4.815 | 1.00 | 75.96 | C |
| ATOM | 6395 | OG1 | THR | B | 326 | −35.302 | 17.962 | 4.508 | 1.00 | 80.83 | O |
| ATOM | 6396 | CG2 | THR | B | 326 | −33.922 | 16.425 | 5.741 | 1.00 | 79.15 | C |
| ATOM | 6397 | C | THR | B | 326 | −34.514 | 14.344 | 3.894 | 1.00 | 75.82 | C |
| ATOM | 6398 | O | THR | B | 326 | −33.338 | 14.001 | 3.875 | 1.00 | 75.03 | O |
| ATOM | 6399 | N | TYR | B | 327 | −35.510 | 13.523 | 4.292 | 1.00 | 73.88 | N |
| ATOM | 6400 | CA | TYR | B | 327 | −35.262 | 12.121 | 4.660 | 1.00 | 67.64 | C |
| ATOM | 6401 | CB | TYR | B | 327 | −36.494 | 11.473 | 5.314 | 1.00 | 59.73 | C |
| ATOM | 6402 | CG | TYR | B | 327 | −36.863 | 12.105 | 6.640 | 1.00 | 57.05 | C |
| ATOM | 6403 | CD2 | TYR | B | 327 | −37.719 | 13.194 | 6.690 | 1.00 | 57.61 | C |
| ATOM | 6404 | CE2 | TYR | B | 327 | −38.049 | 13.799 | 7.890 | 1.00 | 53.56 | C |
| ATOM | 6405 | CZ | TYR | B | 327 | −37.518 | 13.316 | 9.066 | 1.00 | 53.67 | C |
| ATOM | 6406 | OH | TYR | B | 327 | −37.846 | 13.921 | 10.258 | 1.00 | 56.47 | O |
| ATOM | 6407 | CE1 | TYR | B | 327 | −36.661 | 12.235 | 9.050 | 1.00 | 52.33 | C |
| ATOM | 6408 | CD1 | TYR | B | 327 | −36.335 | 11.636 | 7.839 | 1.00 | 54.45 | C |
| ATOM | 6409 | C | TYR | B | 327 | −34.774 | 11.296 | 3.489 | 1.00 | 68.29 | C |
| ATOM | 6410 | O | TYR | B | 327 | −33.872 | 10.494 | 3.632 | 1.00 | 71.56 | O |
| ATOM | 6411 | N | MET | B | 328 | −35.369 | 11.498 | 2.312 | 1.00 | 71.04 | N |
| ATOM | 6412 | CA | MET | B | 328 | −34.893 | 10.807 | 1.129 | 1.00 | 63.69 | C |
| ATOM | 6413 | CB | MET | B | 328 | −35.726 | 11.190 | −0.095 | 1.00 | 68.38 | C |
| ATOM | 6414 | CG | MET | B | 328 | −37.137 | 10.639 | −0.101 | 1.00 | 67.95 | C |
| ATOM | 6415 | SD | MET | B | 328 | −38.052 | 11.191 | −1.554 | 1.00 | 92.87 | S |
| ATOM | 6416 | CE | MET | B | 328 | −37.036 | 10.531 | −2.874 | 1.00 | 80.30 | C |
| ATOM | 6417 | C | MET | B | 328 | −33.438 | 11.104 | 0.853 | 1.00 | 63.27 | C |
| ATOM | 6418 | O | MET | B | 328 | −32.665 | 10.233 | 0.528 | 1.00 | 75.33 | O |
| ATOM | 6419 | N | LEU | B | 329 | −33.082 | 12.369 | 0.930 | 1.00 | 67.24 | N |
| ATOM | 6420 | CA | LEU | B | 329 | −31.733 | 12.763 | 0.597 | 1.00 | 82.08 | C |
| ATOM | 6421 | CB | LEU | B | 329 | −31.657 | 14.278 | 0.381 | 1.00 | 84.17 | C |
| ATOM | 6422 | CG | LEU | B | 329 | −32.412 | 14.810 | −0.846 | 1.00 | 84.28 | C |
| ATOM | 6423 | CD1 | LEU | B | 329 | −32.084 | 16.276 | −1.105 | 1.00 | 82.23 | C |
| ATOM | 6424 | CD2 | LEU | B | 329 | −32.135 | 13.962 | −2.086 | 1.00 | 76.85 | C |
| ATOM | 6425 | C | LEU | B | 329 | −30.699 | 12.299 | 1.626 | 1.00 | 84.59 | C |
| ATOM | 6426 | O | LEU | B | 329 | −29.644 | 11.791 | 1.265 | 1.00 | 92.51 | O |
| ATOM | 6427 | N | ALA | B | 330 | −31.016 | 12.469 | 2.909 | 1.00 | 86.28 | N |
| ATOM | 6428 | CA | ALA | B | 330 | −30.103 | 12.097 | 3.996 | 1.00 | 85.18 | C |
| ATOM | 6429 | CB | ALA | B | 330 | −30.707 | 12.494 | 5.340 | 1.00 | 88.55 | C |
| ATOM | 6430 | C | ALA | B | 330 | −29.673 | 10.629 | 4.026 | 1.00 | 74.96 | C |
| ATOM | 6431 | O | ALA | B | 330 | −28.895 | 10.282 | 4.944 | 1.00 | 74.17 | O |
| ATOM | 6432 | N | VAL | B | 343 | −24.161 | 22.199 | −2.342 | 1.00 | 85.44 | N |
| ATOM | 6433 | CA | VAL | B | 343 | −25.543 | 22.418 | −2.837 | 1.00 | 98.24 | C |
| ATOM | 6434 | CB | VAL | B | 343 | −25.718 | 21.959 | −4.291 | 1.00 | 105.59 | C |
| ATOM | 6435 | CG1 | VAL | B | 343 | −27.201 | 21.867 | −4.646 | 1.00 | 93.48 | C |
| ATOM | 6436 | CG2 | VAL | B | 343 | −24.987 | 22.902 | −5.233 | 1.00 | 110.62 | C |
| ATOM | 6437 | C | VAL | B | 343 | −26.497 | 21.626 | −1.939 | 1.00 | 98.02 | C |
| ATOM | 6438 | O | VAL | B | 343 | −27.400 | 22.176 | −1.332 | 1.00 | 98.77 | O |
| ATOM | 6439 | N | PHE | B | 344 | −26.197 | 20.335 | −1.790 | 1.00 | 103.38 | N |
| ATOM | 6440 | CA | PHE | B | 344 | −26.890 | 19.445 | −0.880 | 1.00 | 94.11 | C |
| ATOM | 6441 | CB | PHE | B | 344 | −26.283 | 18.050 | −1.061 | 1.00 | 104.08 | C |
| ATOM | 6442 | CG | PHE | B | 344 | −26.826 | 17.001 | −0.136 | 1.00 | 101.98 | C |
| ATOM | 6443 | CD1 | PHE | B | 344 | −28.032 | 16.375 | −0.412 | 1.00 | 107.83 | C |
| ATOM | 6444 | CE1 | PHE | B | 344 | −28.520 | 15.389 | 0.422 | 1.00 | 103.60 | C |
| ATOM | 6445 | CZ | PHE | B | 344 | −27.796 | 15.003 | 1.539 | 1.00 | 100.98 | C |
| ATOM | 6446 | CE2 | PHE | B | 344 | −26.581 | 15.608 | 1.816 | 1.00 | 91.16 | C |
| ATOM | 6447 | CD2 | PHE | B | 344 | −26.099 | 16.595 | 0.975 | 1.00 | 91.20 | C |
| ATOM | 6448 | C | PHE | B | 344 | −26.682 | 19.927 | 0.546 | 1.00 | 86.54 | C |
| ATOM | 6449 | O | PHE | B | 344 | −27.561 | 19.876 | 1.374 | 1.00 | 86.69 | O |
| ATOM | 6450 | N | ILE | B | 345 | −25.470 | 20.351 | 0.814 | 1.00 | 85.81 | N |
| ATOM | 6451 | CA | ILE | B | 345 | −25.088 | 20.644 | 2.150 | 1.00 | 78.93 | C |
| ATOM | 6452 | CB | ILE | B | 345 | −23.626 | 21.113 | 2.199 | 1.00 | 86.31 | C |
| ATOM | 6453 | CG1 | ILE | B | 345 | −22.707 | 20.057 | 1.579 | 1.00 | 87.08 | C |
| ATOM | 6454 | CD1 | ILE | B | 345 | −21.289 | 20.539 | 1.365 | 1.00 | 84.63 | C |
| ATOM | 6455 | CG2 | ILE | B | 345 | −23.218 | 21.442 | 3.629 | 1.00 | 85.24 | C |
| ATOM | 6456 | C | ILE | B | 345 | −25.926 | 21.751 | 2.690 | 1.00 | 78.09 | C |
| ATOM | 6457 | O | ILE | B | 345 | −26.390 | 21.677 | 3.797 | 1.00 | 77.07 | O |
| ATOM | 6458 | N | TYR | B | 346 | −26.082 | 22.803 | 1.905 | 1.00 | 81.04 | N |
| ATOM | 6459 | CA | TYR | B | 346 | −26.962 | 23.912 | 2.230 | 1.00 | 76.17 | C |
| ATOM | 6460 | CB | TYR | B | 346 | −26.548 | 25.174 | 1.465 | 1.00 | 80.97 | C |
| ATOM | 6461 | CG | TYR | B | 346 | −25.158 | 25.628 | 1.858 | 1.00 | 83.57 | C |
| ATOM | 6462 | CD1 | TYR | B | 346 | −24.963 | 26.490 | 2.929 | 1.00 | 78.07 | C |
| ATOM | 6463 | CE1 | TYR | B | 346 | −23.691 | 26.891 | 3.302 | 1.00 | 83.38 | C |
| ATOM | 6464 | CZ | TYR | B | 346 | −22.594 | 26.418 | 2.608 | 1.00 | 88.05 | C |
| ATOM | 6465 | OH | TYR | B | 346 | −21.327 | 26.809 | 2.972 | 1.00 | 83.11 | O |
| ATOM | 6466 | CE2 | TYR | B | 346 | −22.761 | 25.554 | 1.549 | 1.00 | 88.87 | C |
| ATOM | 6467 | CD2 | TYR | B | 346 | −24.037 | 25.160 | 1.182 | 1.00 | 89.06 | C |
| ATOM | 6468 | C | TYR | B | 346 | −28.434 | 23.638 | 2.156 | 1.00 | 71.55 | C |
| ATOM | 6469 | O | TYR | B | 346 | −29.180 | 24.066 | 2.993 | 1.00 | 72.02 | O |
| ATOM | 6470 | N | PHE | B | 347 | −28.850 | 22.917 | 1.139 | 1.00 | 74.13 | N |
| ATOM | 6471 | CA | PHE | B | 347 | −30.253 | 22.633 | 0.942 | 1.00 | 71.20 | C |
| ATOM | 6472 | CB | PHE | B | 347 | −30.414 | 21.870 | −0.373 | 1.00 | 64.88 | C |
| ATOM | 6473 | CG | PHE | B | 347 | −31.826 | 21.477 | −0.693 | 1.00 | 68.89 | C |
| ATOM | 6474 | CD1 | PHE | B | 347 | −32.708 | 22.388 | −1.250 | 1.00 | 64.90 | C |
| ATOM | 6475 | CE1 | PHE | B | 347 | −34.007 | 22.017 | −1.563 | 1.00 | 59.46 | C |
| ATOM | 6476 | CZ | PHE | B | 347 | −34.430 | 20.725 | −1.332 | 1.00 | 68.58 | C |
| ATOM | 6477 | CE2 | PHE | B | 347 | −33.558 | 19.805 | −0.788 | 1.00 | 78.09 | C |
| ATOM | 6478 | CD2 | PHE | B | 347 | −32.261 | 20.181 | −0.476 | 1.00 | 74.21 | C |
| ATOM | 6479 | C | PHE | B | 347 | −30.775 | 21.818 | 2.091 | 1.00 | 72.08 | C |
| ATOM | 6480 | O | PHE | B | 347 | −31.876 | 21.988 | 2.541 | 1.00 | 72.48 | O |
| ATOM | 6481 | N | ASN | B | 348 | −29.969 | 20.899 | 2.549 | 1.00 | 71.43 | N |
| ATOM | 6482 | CA | ASN | B | 348 | −30.324 | 20.082 | 3.698 | 1.00 | 66.83 | C |
| ATOM | 6483 | CB | ASN | B | 348 | −29.265 | 19.005 | 3.942 | 1.00 | 70.96 | C |
| ATOM | 6484 | CG | ASN | B | 348 | −29.803 | 17.830 | 4.735 | 1.00 | 76.33 | C |
| ATOM | 6485 | OD1 | ASN | B | 348 | −30.933 | 17.385 | 4.523 | 1.00 | 81.22 | O |
| ATOM | 6486 | ND2 | ASN | B | 348 | −28.995 | 17.321 | 5.658 | 1.00 | 87.33 | N |
| ATOM | 6487 | C | ASN | B | 348 | −30.507 | 20.919 | 4.957 | 1.00 | 64.01 | C |
| ATOM | 6488 | O | ASN | B | 348 | −31.379 | 20.669 | 5.764 | 1.00 | 68.33 | O |
| ATOM | 6489 | N | ALA | B | 349 | −29.611 | 21.868 | 5.172 | 1.00 | 64.37 | N |
| ATOM | 6490 | CA | ALA | B | 349 | −29.648 | 22.631 | 6.396 | 1.00 | 63.41 | C |
| ATOM | 6491 | CB | ALA | B | 349 | −28.331 | 23.371 | 6.603 | 1.00 | 62.70 | C |
| ATOM | 6492 | C | ALA | B | 349 | −30.805 | 23.602 | 6.391 | 1.00 | 56.79 | C |
| ATOM | 6493 | O | ALA | B | 349 | −31.477 | 23.744 | 7.392 | 1.00 | 59.56 | O |
| ATOM | 6494 | N | PHE | B | 350 | −31.184 | 24.065 | 5.208 | 1.00 | 53.65 | N |
| ATOM | 6495 | CA | PHE | B | 350 | −32.387 | 24.840 | 5.082 | 1.00 | 49.43 | C |
| ATOM | 6496 | CB | PHE | B | 350 | −32.528 | 25.282 | 3.618 | 1.00 | 53.91 | C |
| ATOM | 6497 | CG | PHE | B | 350 | −33.874 | 25.843 | 3.265 | 1.00 | 51.58 | C |
| ATOM | 6498 | CD1 | PHE | B | 350 | −34.360 | 26.982 | 3.888 | 1.00 | 51.08 | C |
| ATOM | 6499 | CE1 | PHE | B | 350 | −35.602 | 27.496 | 3.551 | 1.00 | 50.57 | C |
| ATOM | 6500 | CZ | PHE | B | 350 | −36.360 | 26.875 | 2.573 | 1.00 | 52.36 | C |
| ATOM | 6501 | CE2 | PHE | B | 350 | −35.877 | 25.750 | 1.939 | 1.00 | 49.29 | C |
| ATOM | 6502 | CD2 | PHE | B | 350 | −34.642 | 25.243 | 2.282 | 1.00 | 47.66 | C |
| ATOM | 6503 | C | PHE | B | 350 | −33.624 | 24.052 | 5.544 | 1.00 | 50.70 | C |
| ATOM | 6504 | O | PHE | B | 350 | −34.352 | 24.479 | 6.408 | 1.00 | 51.78 | O |
| ATOM | 6505 | N | LEU | B | 351 | −33.868 | 22.900 | 4.943 | 1.00 | 50.64 | N |
| ATOM | 6506 | CA | LEU | B | 351 | −35.057 | 22.134 | 5.275 | 1.00 | 49.05 | C |
| ATOM | 6507 | CB | LEU | B | 351 | −35.316 | 21.085 | 4.199 | 1.00 | 53.50 | C |
| ATOM | 6508 | CG | LEU | B | 351 | −35.644 | 21.740 | 2.861 | 1.00 | 49.61 | C |
| ATOM | 6509 | CD1 | LEU | B | 351 | −35.838 | 20.696 | 1.793 | 1.00 | 47.77 | C |
| ATOM | 6510 | CD2 | LEU | B | 351 | −36.881 | 22.613 | 3.000 | 1.00 | 43.03 | C |
| ATOM | 6511 | C | LEU | B | 351 | −35.077 | 21.504 | 6.658 | 1.00 | 53.23 | C |
| ATOM | 6512 | O | LEU | B | 351 | −36.117 | 21.404 | 7.273 | 1.00 | 59.27 | O |
| ATOM | 6513 | N | GLU | B | 352 | −33.940 | 21.021 | 7.139 | 1.00 | 57.50 | N |
| ATOM | 6514 | CA | GLU | B | 352 | −33.874 | 20.443 | 8.483 | 1.00 | 60.39 | C |
| ATOM | 6515 | CB | GLU | B | 352 | −32.507 | 19.806 | 8.759 | 1.00 | 69.00 | C |
| ATOM | 6516 | CG | GLU | B | 352 | −32.446 | 18.352 | 8.304 | 1.00 | 76.46 | C |
| ATOM | 6517 | CD | GLU | B | 352 | −31.041 | 17.785 | 8.270 | 1.00 | 90.27 | C |
| ATOM | 6518 | OE1 | GLU | B | 352 | −30.106 | 18.466 | 8.744 | 1.00 | 90.19 | O |
| ATOM | 6519 | OE2 | GLU | B | 352 | −30.878 | 16.653 | 7.763 | 1.00 | 90.15 | O |
| ATOM | 6520 | C | GLU | B | 352 | −34.277 | 21.454 | 9.550 | 1.00 | 53.26 | C |
| ATOM | 6521 | O | GLU | B | 352 | −35.017 | 21.162 | 10.469 | 1.00 | 54.97 | O |
| ATOM | 6522 | N | SER | B | 353 | −33.773 | 22.665 | 9.395 | 1.00 | 48.75 | N |
| ATOM | 6523 | CA | SER | B | 353 | −34.071 | 23.737 | 10.310 | 1.00 | 49.38 | C |
| ATOM | 6524 | CB | SER | B | 353 | −33.057 | 24.877 | 10.157 | 1.00 | 46.31 | C |
| ATOM | 6525 | OG | SER | B | 353 | −33.060 | 25.392 | 8.841 | 1.00 | 47.02 | O |
| ATOM | 6526 | C | SER | B | 353 | −35.503 | 24.279 | 10.208 | 1.00 | 50.98 | C |
| ATOM | 6527 | O | SER | B | 353 | −36.074 | 24.687 | 11.199 | 1.00 | 50.20 | O |
| ATOM | 6528 | N | PHE | B | 354 | −36.024 | 24.351 | 8.985 | 1.00 | 43.47 | N |
| ATOM | 6529 | CA | PHE | B | 354 | −37.326 | 24.926 | 8.694 | 1.00 | 39.95 | C |
| ATOM | 6530 | CB | PHE | B | 354 | −37.360 | 25.590 | 7.318 | 1.00 | 37.91 | C |
| ATOM | 6531 | CG | PHE | B | 354 | −37.726 | 27.046 | 7.370 | 1.00 | 39.92 | C |
| ATOM | 6532 | CD1 | PHE | B | 354 | −37.369 | 27.825 | 8.464 | 1.00 | 36.79 | C |
| ATOM | 6533 | CE1 | PHE | B | 354 | −37.709 | 29.167 | 8.521 | 1.00 | 31.13 | C |
| ATOM | 6534 | CZ | PHE | B | 354 | −38.423 | 29.738 | 7.487 | 1.00 | 32.75 | C |
| ATOM | 6535 | CE2 | PHE | B | 354 | −38.792 | 28.970 | 6.399 | 1.00 | 34.09 | C |
| ATOM | 6536 | CD2 | PHE | B | 354 | −38.448 | 27.632 | 6.346 | 1.00 | 38.55 | C |
| ATOM | 6537 | C | PHE | B | 354 | −38.505 | 23.973 | 8.860 | 1.00 | 48.32 | C |
| ATOM | 6538 | O | PHE | B | 354 | −39.641 | 24.375 | 8.706 | 1.00 | 46.23 | O |
| ATOM | 6539 | N | GLN | B | 355 | −38.249 | 22.707 | 9.152 | 1.00 | 46.99 | N |
| ATOM | 6540 | CA | GLN | B | 355 | −39.320 | 21.731 | 9.333 | 1.00 | 46.38 | C |
| ATOM | 6541 | CB | GLN | B | 355 | −38.754 | 20.366 | 9.736 | 1.00 | 51.18 | C |
| ATOM | 6542 | CG | GLN | B | 355 | −38.284 | 19.487 | 8.587 | 1.00 | 68.03 | C |
| ATOM | 6543 | CD | GLN | B | 355 | −37.826 | 18.128 | 9.078 | 1.00 | 83.68 | C |
| ATOM | 6544 | OE1 | GLN | B | 355 | −37.858 | 17.856 | 10.280 | 1.00 | 90.16 | O |
| ATOM | 6545 | NE2 | GLN | B | 355 | −37.401 | 17.267 | 8.155 | 1.00 | 84.34 | N |
| ATOM | 6546 | C | GLN | B | 355 | −40.370 | 22.155 | 10.360 | 1.00 | 42.51 | C |
| ATOM | 6547 | O | GLN | B | 355 | −41.555 | 22.009 | 10.146 | 1.00 | 45.29 | O |
| ATOM | 6548 | N | GLY | B | 356 | −39.931 | 22.745 | 11.444 | 1.00 | 43.17 | N |
| ATOM | 6549 | CA | GLY | B | 356 | −40.805 | 23.306 | 12.438 | 1.00 | 40.45 | C |
| ATOM | 6550 | C | GLY | B | 356 | −41.704 | 24.391 | 11.926 | 1.00 | 39.25 | C |
| ATOM | 6551 | O | GLY | B | 356 | −42.870 | 24.366 | 12.214 | 1.00 | 39.52 | O |
| ATOM | 6552 | N | PHE | B | 357 | −41.196 | 25.326 | 11.130 | 1.00 | 39.66 | N |
| ATOM | 6553 | CA | PHE | B | 357 | −42.018 | 26.384 | 10.514 | 1.00 | 39.41 | C |
| ATOM | 6554 | CB | PHE | B | 357 | −41.111 | 27.329 | 9.720 | 1.00 | 33.26 | C |
| ATOM | 6555 | CG | PHE | B | 357 | −41.831 | 28.477 | 9.073 | 1.00 | 34.48 | C |
| ATOM | 6556 | CD1 | PHE | B | 357 | −42.098 | 29.633 | 9.783 | 1.00 | 37.48 | C |
| ATOM | 6557 | CE1 | PHE | B | 357 | −42.751 | 30.697 | 9.183 | 1.00 | 36.80 | C |
| ATOM | 6558 | CZ | PHE | B | 357 | −43.137 | 30.613 | 7.861 | 1.00 | 33.09 | C |
| ATOM | 6559 | CE2 | PHE | B | 357 | −42.869 | 29.471 | 7.139 | 1.00 | 34.51 | C |
| ATOM | 6560 | CD2 | PHE | B | 357 | −42.216 | 28.412 | 7.743 | 1.00 | 36.62 | C |
| ATOM | 6561 | C | PHE | B | 357 | −43.112 | 25.810 | 9.607 | 1.00 | 38.99 | C |
| ATOM | 6562 | O | PHE | B | 357 | −44.233 | 26.266 | 9.591 | 1.00 | 34.72 | O |
| ATOM | 6563 | N | PHE | B | 358 | −42.761 | 24.802 | 8.824 | 1.00 | 40.78 | N |
| ATOM | 6564 | CA | PHE | B | 358 | −43.719 | 24.155 | 7.961 | 1.00 | 35.64 | C |
| ATOM | 6565 | CB | PHE | B | 358 | −43.038 | 23.049 | 7.155 | 1.00 | 35.22 | C |
| ATOM | 6566 | CG | PHE | B | 358 | −42.007 | 23.556 | 6.191 | 1.00 | 40.39 | C |
| ATOM | 6567 | CD1 | PHE | B | 358 | −42.224 | 24.719 | 5.475 | 1.00 | 40.87 | C |
| ATOM | 6568 | CE1 | PHE | B | 358 | −41.274 | 25.192 | 4.592 | 1.00 | 38.27 | C |
| ATOM | 6569 | CZ | PHE | B | 358 | −40.093 | 24.507 | 4.420 | 1.00 | 44.87 | C |
| ATOM | 6570 | CE2 | PHE | B | 358 | −39.863 | 23.352 | 5.128 | 1.00 | 44.80 | C |
| ATOM | 6571 | CD2 | PHE | B | 358 | −40.816 | 22.882 | 6.010 | 1.00 | 42.74 | C |
| ATOM | 6572 | C | PHE | B | 358 | −44.887 | 23.581 | 8.738 | 1.00 | 41.25 | C |
| ATOM | 6573 | O | PHE | B | 358 | −46.027 | 23.795 | 8.403 | 1.00 | 48.48 | O |
| ATOM | 6574 | N | VAL | B | 359 | −44.605 | 22.893 | 9.822 | 1.00 | 37.76 | N |
| ATOM | 6575 | CA | VAL | B | 359 | −45.646 | 22.397 | 10.697 | 1.00 | 36.81 | C |
| ATOM | 6576 | CB | VAL | B | 359 | −45.049 | 21.597 | 11.876 | 1.00 | 32.24 | C |
| ATOM | 6577 | CG1 | VAL | B | 359 | −46.127 | 21.212 | 12.881 | 1.00 | 37.62 | C |
| ATOM | 6578 | CG2 | VAL | B | 359 | −44.357 | 20.360 | 11.355 | 1.00 | 30.45 | C |
| ATOM | 6579 | C | VAL | B | 359 | −46.577 | 23.490 | 11.209 | 1.00 | 41.26 | C |
| ATOM | 6580 | O | VAL | B | 359 | −47.766 | 23.333 | 11.176 | 1.00 | 45.81 | O |
| ATOM | 6581 | N | SER | B | 360 | −46.049 | 24.614 | 11.646 | 1.00 | 35.66 | N |
| ATOM | 6582 | CA | SER | B | 360 | −46.877 | 25.713 | 12.121 | 1.00 | 37.27 | C |
| ATOM | 6583 | CB | SER | B | 360 | −46.029 | 26.840 | 12.718 | 1.00 | 36.05 | C |
| ATOM | 6584 | OG | SER | B | 360 | −45.354 | 27.560 | 11.707 | 1.00 | 49.21 | O |
| ATOM | 6585 | C | SER | B | 360 | −47.835 | 26.251 | 11.092 | 1.00 | 38.18 | C |
| ATOM | 6586 | O | SER | B | 360 | −48.986 | 26.465 | 11.380 | 1.00 | 43.63 | O |
| ATOM | 6587 | N | VAL | B | 361 | −47.367 | 26.422 | 9.874 | 1.00 | 37.88 | N |
| ATOM | 6588 | CA | VAL | B | 361 | −48.221 | 26.835 | 8.783 | 1.00 | 41.10 | C |
| ATOM | 6589 | CB | VAL | B | 361 | −47.415 | 26.999 | 7.479 | 1.00 | 41.69 | C |
| ATOM | 6590 | CG1 | VAL | B | 361 | −48.327 | 27.413 | 6.329 | 1.00 | 37.69 | C |
| ATOM | 6591 | CG2 | VAL | B | 361 | −46.294 | 28.006 | 7.675 | 1.00 | 39.33 | C |
| ATOM | 6592 | C | VAL | B | 361 | −49.314 | 25.820 | 8.542 | 1.00 | 47.74 | C |
| ATOM | 6593 | O | VAL | B | 361 | −50.460 | 26.156 | 8.357 | 1.00 | 53.57 | O |
| ATOM | 6594 | N | PHE | B | 362 | −48.937 | 24.561 | 8.536 | 1.00 | 45.04 | N |
| ATOM | 6595 | CA | PHE | B | 362 | −49.871 | 23.489 | 8.314 | 1.00 | 46.63 | C |
| ATOM | 6596 | CB | PHE | B | 362 | −49.059 | 22.183 | 8.234 | 1.00 | 43.78 | C |
| ATOM | 6597 | CG | PHE | B | 362 | −49.875 | 20.928 | 8.316 | 1.00 | 42.99 | C |
| ATOM | 6598 | CD1 | PHE | B | 362 | −50.552 | 20.448 | 7.210 | 1.00 | 40.68 | C |
| ATOM | 6599 | CE1 | PHE | B | 362 | −51.289 | 19.284 | 7.287 | 1.00 | 43.75 | C |
| ATOM | 6600 | CZ | PHE | B | 362 | −51.340 | 18.577 | 8.473 | 1.00 | 49.51 | C |
| ATOM | 6601 | CE2 | PHE | B | 362 | −50.656 | 19.037 | 9.578 | 1.00 | 50.16 | C |
| ATOM | 6602 | CD2 | PHE | B | 362 | −49.923 | 20.201 | 9.494 | 1.00 | 49.22 | C |
| ATOM | 6603 | C | PHE | B | 362 | −50.979 | 23.413 | 9.369 | 1.00 | 44.31 | C |
| ATOM | 6604 | O | PHE | B | 362 | −52.142 | 23.297 | 9.056 | 1.00 | 44.13 | O |
| ATOM | 6605 | N | ALA | B | 363 | −50.589 | 23.482 | 10.630 | 1.00 | 45.30 | N |
| ATOM | 6606 | CA | ALA | B | 363 | −51.507 | 23.485 | 11.758 | 1.00 | 50.72 | C |
| ATOM | 6607 | CB | ALA | B | 363 | −50.732 | 23.402 | 13.067 | 1.00 | 46.02 | C |
| ATOM | 6608 | C | ALA | B | 363 | −52.448 | 24.685 | 11.778 | 1.00 | 59.77 | C |
| ATOM | 6609 | O | ALA | B | 363 | −53.651 | 24.562 | 11.964 | 1.00 | 67.25 | O |
| ATOM | 6610 | N | CYS | B | 364 | −51.857 | 25.845 | 11.535 | 1.00 | 52.82 | N |
| ATOM | 6611 | CA | CYS | B | 364 | −52.563 | 27.107 | 11.424 | 1.00 | 60.45 | C |
| ATOM | 6612 | CB | CYS | B | 364 | −51.586 | 28.286 | 11.386 | 1.00 | 62.96 | C |
| ATOM | 6613 | SG | CYS | B | 364 | −50.903 | 28.713 | 13.014 | 1.00 | 65.72 | S |
| ATOM | 6614 | C | CYS | B | 364 | −53.551 | 27.155 | 10.231 | 1.00 | 64.68 | C |
| ATOM | 6615 | O | CYS | B | 364 | −54.679 | 27.641 | 10.344 | 1.00 | 69.09 | O |
| ATOM | 6616 | N | PHE | B | 365 | −53.145 | 26.575 | 9.111 | 1.00 | 57.49 | N |
| ATOM | 6617 | CA | PHE | B | 365 | −54.039 | 26.386 | 7.969 | 1.00 | 59.93 | C |
| ATOM | 6618 | CB | PHE | B | 365 | −53.280 | 25.678 | 6.845 | 1.00 | 55.65 | C |
| ATOM | 6619 | CG | PHE | B | 365 | −54.133 | 25.301 | 5.671 | 1.00 | 57.08 | C |
| ATOM | 6620 | CD1 | PHE | B | 365 | −54.699 | 26.276 | 4.869 | 1.00 | 65.62 | C |
| ATOM | 6621 | CE1 | PHE | B | 365 | −55.478 | 25.933 | 3.779 | 1.00 | 67.94 | C |
| ATOM | 6622 | CZ | PHE | B | 365 | −55.688 | 24.604 | 3.474 | 1.00 | 61.37 | C |
| ATOM | 6623 | CE2 | PHE | B | 365 | −55.122 | 23.622 | 4.259 | 1.00 | 58.36 | C |
| ATOM | 6624 | CD2 | PHE | B | 365 | −54.346 | 23.972 | 5.351 | 1.00 | 57.13 | C |
| ATOM | 6625 | C | PHE | B | 365 | −55.311 | 25.584 | 8.335 | 1.00 | 65.78 | C |
| ATOM | 6626 | O | PHE | B | 365 | −56.428 | 26.010 | 8.079 | 1.00 | 74.67 | O |
| ATOM | 6627 | N | LEU | B | 366 | −55.125 | 24.416 | 8.944 | 1.00 | 58.32 | N |
| ATOM | 6628 | CA | LEU | B | 366 | −56.251 | 23.550 | 9.247 | 1.00 | 66.90 | C |
| ATOM | 6629 | CB | LEU | B | 366 | −55.761 | 22.166 | 9.677 | 1.00 | 61.24 | C |
| ATOM | 6630 | CG | LEU | B | 366 | −54.966 | 21.403 | 8.616 | 1.00 | 57.89 | C |
| ATOM | 6631 | CD1 | LEU | B | 366 | −54.517 | 20.046 | 9.138 | 1.00 | 47.54 | C |
| ATOM | 6632 | CD2 | LEU | B | 366 | −55.785 | 21.248 | 7.343 | 1.00 | 63.44 | C |
| ATOM | 6633 | C | LEU | B | 366 | −57.206 | 24.126 | 10.305 | 1.00 | 78.42 | C |
| ATOM | 6634 | O | LEU | B | 366 | −58.418 | 24.043 | 10.174 | 1.00 | 88.50 | O |
| ATOM | 6635 | N | ASN | B | 367 | −56.677 | 24.722 | 11.357 | 1.00 | 71.56 | N |
| ATOM | 6636 | CA | ASN | B | 367 | −57.553 | 25.333 | 12.350 | 1.00 | 76.47 | C |
| ATOM | 6637 | CB | ASN | B | 367 | −56.745 | 25.807 | 13.556 | 1.00 | 78.51 | C |
| ATOM | 6638 | CG | ASN | B | 367 | −57.624 | 26.193 | 14.726 | 1.00 | 81.31 | C |
| ATOM | 6639 | OD1 | ASN | B | 367 | −58.815 | 25.876 | 14.752 | 1.00 | 86.74 | O |
| ATOM | 6640 | ND2 | ASN | B | 367 | −57.040 | 26.873 | 15.707 | 1.00 | 73.67 | N |
| ATOM | 6641 | C | ASN | B | 367 | −58.412 | 26.499 | 11.793 | 1.00 | 87.33 | C |
| ATOM | 6642 | O | ASN | B | 367 | −59.629 | 26.510 | 11.971 | 1.00 | 92.71 | O |
| ATOM | 6643 | N | SER | B | 368 | −57.749 | 27.480 | 11.140 | 1.00 | 87.17 | N |
| ATOM | 6644 | CA | SER | B | 368 | −58.415 | 28.694 | 10.592 | 1.00 | 94.99 | C |
| ATOM | 6645 | CB | SER | B | 368 | −59.127 | 28.372 | 9.273 | 1.00 | 94.47 | C |
| ATOM | 6646 | OG | SER | B | 368 | −60.399 | 27.785 | 9.505 | 1.00 | 104.43 | O |
| ATOM | 6647 | C | SER | B | 368 | −59.403 | 29.447 | 11.531 | 1.00 | 100.50 | C |
| ATOM | 6648 | O | SER | B | 368 | −60.247 | 30.227 | 11.007 | 1.00 | 94.65 | O |
| ATOM | 6649 | C24 | CP3 | B | 900 | −42.539 | 14.640 | 10.561 | 1.00 | 38.03 | C |
| ATOM | 6650 | C23 | CP3 | B | 900 | −41.557 | 14.048 | 9.582 | 1.00 | 40.36 | C |
| ATOM | 6651 | C20 | CP3 | B | 900 | −42.262 | 13.124 | 8.615 | 1.00 | 47.49 | C |
| ATOM | 6652 | C21 | CP3 | B | 900 | −41.335 | 12.859 | 7.448 | 1.00 | 47.79 | C |
| ATOM | 6653 | C22 | CP3 | B | 900 | −42.133 | 12.540 | 6.212 | 1.00 | 48.96 | C |
| ATOM | 6654 | N19 | CP3 | B | 900 | −43.494 | 13.738 | 8.180 | 1.00 | 48.74 | N |
| ATOM | 6655 | C15 | CP3 | B | 900 | −44.622 | 13.008 | 8.107 | 1.00 | 39.92 | C |
| ATOM | 6656 | C14 | CP3 | B | 900 | −44.776 | 11.826 | 8.803 | 1.00 | 38.15 | C |
| ATOM | 6657 | C13 | CP3 | B | 900 | −45.946 | 11.098 | 8.711 | 1.00 | 40.46 | C |
| ATOM | 6658 | C18 | CP3 | B | 900 | −46.066 | 9.824 | 9.484 | 1.00 | 39.05 | C |
| ATOM | 6659 | N12 | CP3 | B | 900 | −46.980 | 11.492 | 7.954 | 1.00 | 47.16 | N |
| ATOM | 6660 | C16 | CP3 | B | 900 | −45.737 | 13.458 | 7.274 | 1.00 | 43.14 | C |
| ATOM | 6661 | C17 | CP3 | B | 900 | −45.645 | 14.733 | 6.489 | 1.00 | 51.56 | C |
| ATOM | 6662 | C11 | CP3 | B | 900 | −46.936 | 12.616 | 7.244 | 1.00 | 47.20 | C |
| ATOM | 6663 | O10 | CP3 | B | 900 | −47.987 | 12.992 | 6.490 | 1.00 | 50.44 | O |
| ATOM | 6664 | C1 | CP3 | B | 900 | −49.149 | 12.295 | 6.506 | 1.00 | 50.05 | C |
| ATOM | 6665 | C6 | CP3 | B | 900 | −49.329 | 11.059 | 5.723 | 1.00 | 49.53 | C |
| ATOM | 6666 | C8 | CP3 | B | 900 | −48.204 | 10.538 | 4.880 | 1.00 | 46.24 | C |
| ATOM | 6667 | C5 | CP3 | B | 900 | −50.543 | 10.399 | 5.782 | 1.00 | 49.88 | C |
| ATOM | 6668 | C4 | CP3 | B | 900 | −51.583 | 10.893 | 6.569 | 1.00 | 50.25 | C |
| ATOM | 6669 | C7 | CP3 | B | 900 | −52.893 | 10.161 | 6.621 | 1.00 | 49.21 | C |
| ATOM | 6670 | C3 | CP3 | B | 900 | −51.458 | 12.050 | 7.319 | 1.00 | 48.52 | C |
| ATOM | 6671 | C2 | CP3 | B | 900 | −50.279 | 12.767 | 7.313 | 1.00 | 47.94 | C |
| ATOM | 6672 | C9 | CP3 | B | 900 | −50.134 | 14.015 | 8.118 | 1.00 | 38.66 | C |
| ATOM | 6673 | O1 | POP | B | 901 | −52.482 | 33.064 | 11.773 | 1.00 | 71.53 | O |
| ATOM | 6674 | C2 | POP | B | 901 | −51.536 | 33.402 | 12.468 | 1.00 | 75.51 | C |
| ATOM | 6675 | C3 | POP | B | 901 | −50.140 | 33.439 | 11.902 | 1.00 | 65.62 | C |
| ATOM | 6676 | C4 | POP | B | 901 | −49.262 | 32.450 | 12.654 | 1.00 | 52.73 | C |
| ATOM | 6677 | C5 | POP | B | 901 | −47.925 | 33.073 | 13.033 | 1.00 | 41.16 | C |
| ATOM | 6678 | C6 | POP | B | 901 | −46.811 | 32.057 | 12.863 | 1.00 | 41.83 | C |
| ATOM | 6679 | C7 | POP | B | 901 | −45.591 | 32.656 | 12.183 | 1.00 | 36.59 | C |
| ATOM | 6680 | C8 | POP | B | 901 | −44.798 | 33.539 | 13.139 | 1.00 | 33.34 | C |
| ATOM | 6681 | C9 | POP | B | 901 | −43.327 | 33.132 | 13.121 | 1.00 | 30.71 | C |
| ATOM | 6682 | C10 | POP | B | 901 | −42.423 | 34.350 | 13.231 | 1.00 | 32.28 | C |
| ATOM | 6683 | C11 | POP | B | 901 | −40.937 | 34.011 | 13.342 | 1.00 | 31.17 | C |
| ATOM | 6684 | C12 | POP | B | 901 | −40.239 | 34.977 | 14.299 | 1.00 | 32.48 | C |
| ATOM | 6685 | C13 | POP | B | 901 | −38.719 | 35.048 | 14.117 | 1.00 | 38.01 | C |
| ATOM | 6686 | C14 | POP | B | 901 | −38.008 | 34.947 | 15.466 | 1.00 | 42.70 | C |
| ATOM | 6687 | C15 | POP | B | 901 | −36.742 | 34.096 | 15.510 | 1.00 | 44.20 | C |
| ATOM | 6688 | C16 | POP | B | 901 | −35.813 | 34.555 | 16.642 | 1.00 | 44.04 | C |
| ATOM | 6689 | C17 | POP | B | 901 | −34.439 | 34.916 | 16.119 | 1.00 | 36.57 | C |
| ATOM | 6690 | O | POP | B | 901 | −51.792 | 34.099 | 13.715 | 1.00 | 82.95 | O |
| ATOM | 6691 | C1 | POP | B | 901 | −53.055 | 34.699 | 13.988 | 1.00 | 79.98 | C |
| ATOM | 6692 | C | POP | B | 901 | −52.899 | 35.489 | 15.287 | 1.00 | 72.04 | C |
| ATOM | 6693 | C18 | POP | B | 901 | −54.248 | 35.525 | 15.973 | 1.00 | 77.69 | C |
| ATOM | 6694 | O2 | POP | B | 901 | −55.205 | 35.156 | 14.988 | 1.00 | 80.64 | O |
| ATOM | 6695 | P | POP | B | 901 | −56.256 | 33.983 | 15.280 | 1.00 | 101.63 | P |
| ATOM | 6696 | O5 | POP | B | 901 | −57.632 | 34.810 | 15.314 | 1.00 | 98.25 | O |
| ATOM | 6697 | C19 | POP | B | 901 | −58.859 | 34.179 | 14.960 | 1.00 | 95.60 | C |
| ATOM | 6698 | C20 | POP | B | 901 | −59.619 | 33.805 | 16.227 | 1.00 | 97.22 | C |
| ATOM | 6699 | O6 | POP | B | 901 | −59.097 | 32.802 | 17.108 | 1.00 | 96.58 | O |
| ATOM | 6700 | C21 | POP | B | 901 | −60.930 | 34.512 | 16.560 | 1.00 | 103.28 | C |
| ATOM | 6701 | O7 | POP | B | 901 | −60.815 | 35.228 | 17.796 | 1.00 | 96.27 | O |
| ATOM | 6702 | O3 | POP | B | 901 | −56.272 | 32.864 | 14.128 | 1.00 | 94.33 | O |
| ATOM | 6703 | O4 | POP | B | 901 | −55.996 | 33.373 | 16.601 | 1.00 | 88.88 | O |
| ATOM | 6704 | O8 | POP | B | 901 | −52.591 | 36.842 | 14.979 | 1.00 | 62.29 | O |
| ATOM | 6705 | C22 | POP | B | 901 | −51.202 | 37.185 | 15.235 | 1.00 | 71.57 | C |
| ATOM | 6706 | O9 | POP | B | 901 | −50.998 | 38.205 | 15.866 | 1.00 | 81.64 | O |
| ATOM | 6707 | C23 | POP | B | 901 | −50.045 | 36.239 | 14.986 | 1.00 | 61.30 | C |
| ATOM | 6708 | C24 | POP | B | 901 | −48.765 | 37.050 | 14.781 | 1.00 | 54.82 | C |
| ATOM | 6709 | C25 | POP | B | 901 | −47.958 | 37.359 | 16.057 | 1.00 | 41.99 | C |
| ATOM | 6710 | C26 | POP | B | 901 | −46.492 | 36.957 | 15.861 | 1.00 | 47.22 | C |
| ATOM | 6711 | C27 | POP | B | 901 | −45.334 | 37.620 | 16.593 | 1.00 | 41.96 | C |
| ATOM | 6712 | C28 | POP | B | 901 | −44.355 | 36.563 | 17.136 | 1.00 | 42.45 | C |
| ATOM | 6713 | C29 | POP | B | 901 | −42.977 | 37.076 | 16.761 | 1.00 | 47.01 | C |
| ATOM | 6714 | C30 | POP | B | 901 | −42.681 | 38.338 | 17.516 | 1.00 | 51.48 | C |
| ATOM | 6715 | C31 | POP | B | 901 | −43.088 | 38.476 | 18.767 | 1.00 | 47.54 | C |
| ATOM | 6716 | C32 | POP | B | 901 | −42.970 | 39.847 | 19.378 | 1.00 | 57.34 | C |
| ATOM | 6717 | C33 | POP | B | 901 | −43.850 | 39.985 | 20.625 | 1.00 | 64.65 | C |
| ATOM | 6718 | C34 | POP | B | 901 | −43.356 | 39.089 | 21.760 | 1.00 | 68.25 | C |
| ATOM | 6719 | C35 | POP | B | 901 | −43.927 | 39.489 | 23.120 | 1.00 | 66.66 | C |
| ATOM | 6720 | C36 | POP | B | 901 | −45.396 | 39.881 | 23.016 | 1.00 | 61.00 | C |
| ATOM | 6721 | C37 | POP | B | 901 | −46.052 | 40.165 | 24.360 | 1.00 | 56.40 | C |
| ATOM | 6722 | C38 | POP | B | 901 | −47.522 | 40.503 | 24.163 | 1.00 | 64.13 | C |
| ATOM | 6723 | C39 | POP | B | 901 | −48.383 | 39.268 | 23.997 | 1.00 | 50.54 | C |
| ATOM | 6724 | O1 | POP | B | 902 | −29.616 | 11.174 | 10.046 | 1.00 | 82.20 | O |
| ATOM | 6725 | C2 | POP | B | 902 | −30.353 | 10.402 | 9.451 | 1.00 | 85.06 | C |
| ATOM | 6726 | C3 | POP | B | 902 | −29.781 | 9.383 | 8.499 | 1.00 | 77.48 | C |
| ATOM | 6727 | C4 | POP | B | 902 | −29.870 | 8.000 | 9.126 | 1.00 | 66.86 | C |
| ATOM | 6728 | C5 | POP | B | 902 | −30.110 | 6.929 | 8.072 | 1.00 | 66.09 | C |
| ATOM | 6729 | C6 | POP | B | 902 | −31.181 | 5.964 | 8.545 | 1.00 | 62.77 | C |
| ATOM | 6730 | C7 | POP | B | 902 | −32.229 | 5.722 | 7.471 | 1.00 | 56.87 | C |
| ATOM | 6731 | C8 | POP | B | 902 | −33.541 | 5.238 | 8.077 | 1.00 | 57.17 | C |
| ATOM | 6732 | C9 | POP | B | 902 | −33.873 | 3.849 | 7.542 | 1.00 | 58.88 | C |
| ATOM | 6733 | C10 | POP | B | 902 | −34.733 | 3.064 | 8.522 | 1.00 | 54.70 | C |
| ATOM | 6734 | C11 | POP | B | 902 | −34.446 | 1.564 | 8.505 | 1.00 | 57.41 | C |
| ATOM | 6735 | C12 | POP | B | 902 | −35.740 | 0.762 | 8.370 | 1.00 | 57.08 | C |
| ATOM | 6736 | C13 | POP | B | 902 | −35.525 | −0.750 | 8.234 | 1.00 | 57.50 | C |
| ATOM | 6737 | C14 | POP | B | 902 | −36.289 | −1.502 | 9.322 | 1.00 | 66.89 | C |
| ATOM | 6738 | C15 | POP | B | 902 | −35.563 | −2.659 | 10.001 | 1.00 | 64.90 | C |
| ATOM | 6739 | C16 | POP | B | 902 | −36.008 | −4.001 | 9.406 | 1.00 | 69.57 | C |
| ATOM | 6740 | C17 | POP | B | 902 | −34.835 | −4.932 | 9.189 | 1.00 | 49.44 | C |
| ATOM | 6741 | O | POP | B | 902 | −31.785 | 10.656 | 9.435 | 1.00 | 84.23 | O |
| ATOM | 6742 | C1 | POP | B | 902 | −32.387 | 11.520 | 10.397 | 1.00 | 79.13 | C |
| ATOM | 6743 | C | POP | B | 902 | −33.046 | 10.662 | 11.479 | 1.00 | 73.50 | C |
| ATOM | 6744 | C18 | POP | B | 902 | −32.146 | 10.707 | 12.696 | 1.00 | 71.21 | C |
| ATOM | 6745 | O2 | POP | B | 902 | −31.412 | 11.917 | 12.564 | 1.00 | 87.83 | O |
| ATOM | 6746 | P | POP | B | 902 | −31.836 | 13.245 | 13.355 | 1.00 | 101.45 | P |
| ATOM | 6747 | O5 | POP | B | 902 | −30.445 | 13.620 | 14.070 | 1.00 | 82.26 | O |
| ATOM | 6748 | C19 | POP | B | 902 | −29.448 | 14.387 | 13.401 | 1.00 | 73.44 | C |
| ATOM | 6749 | C20 | POP | B | 902 | −29.884 | 15.841 | 13.271 | 1.00 | 77.23 | C |
| ATOM | 6750 | O6 | POP | B | 902 | −30.410 | 16.542 | 14.406 | 1.00 | 72.80 | O |
| ATOM | 6751 | C21 | POP | B | 902 | −29.756 | 16.548 | 11.925 | 1.00 | 74.85 | C |
| ATOM | 6752 | O7 | POP | B | 902 | −30.058 | 17.942 | 12.061 | 1.00 | 70.00 | O |
| ATOM | 6753 | O3 | POP | B | 902 | −32.332 | 14.407 | 12.362 | 1.00 | 79.76 | O |
| ATOM | 6754 | O4 | POP | B | 902 | −32.876 | 12.950 | 14.365 | 1.00 | 71.03 | O |
| ATOM | 6755 | O8 | POP | B | 902 | −33.066 | 9.303 | 11.057 | 1.00 | 68.28 | O |
| ATOM | 6756 | C22 | POP | B | 902 | −34.409 | 8.784 | 10.850 | 1.00 | 62.85 | C |
| ATOM | 6757 | O9 | POP | B | 902 | −35.305 | 9.407 | 11.389 | 1.00 | 61.72 | O |
| ATOM | 6758 | C23 | POP | B | 902 | −34.784 | 7.865 | 9.707 | 1.00 | 58.50 | C |
| ATOM | 6759 | C24 | POP | B | 902 | −36.174 | 8.265 | 9.207 | 1.00 | 52.52 | C |
| ATOM | 6760 | C25 | POP | B | 902 | −37.312 | 7.268 | 9.497 | 1.00 | 48.16 | C |
| ATOM | 6761 | C26 | POP | B | 902 | −37.989 | 6.866 | 8.182 | 1.00 | 39.62 | C |
| ATOM | 6762 | C27 | POP | B | 902 | −37.245 | 6.585 | 6.885 | 1.00 | 40.20 | C |
| ATOM | 6763 | C28 | POP | B | 902 | −37.985 | 7.238 | 5.702 | 1.00 | 39.11 | C |
| ATOM | 6764 | C29 | POP | B | 902 | −39.135 | 6.296 | 5.383 | 1.00 | 56.48 | C |
| ATOM | 6765 | C30 | POP | B | 902 | −38.794 | 5.457 | 4.187 | 1.00 | 56.76 | C |
| ATOM | 6766 | C31 | POP | B | 902 | −39.675 | 4.589 | 3.712 | 1.00 | 55.34 | C |
| ATOM | 6767 | C32 | POP | B | 902 | −41.052 | 4.570 | 4.322 | 1.00 | 50.53 | C |
| ATOM | 6768 | C33 | POP | B | 902 | −42.129 | 4.373 | 3.252 | 1.00 | 54.44 | C |
| ATOM | 6769 | C34 | POP | B | 902 | −42.527 | 5.709 | 2.626 | 1.00 | 60.03 | C |
| ATOM | 6770 | C35 | POP | B | 902 | −43.611 | 5.561 | 1.560 | 1.00 | 61.20 | C |
| ATOM | 6771 | C36 | POP | B | 902 | −44.401 | 4.274 | 1.765 | 1.00 | 66.34 | C |
| ATOM | 6772 | C37 | POP | B | 902 | −44.074 | 3.174 | 0.765 | 1.00 | 67.83 | C |
| ATOM | 6773 | C38 | POP | B | 902 | −45.004 | 3.262 | −0.435 | 1.00 | 51.65 | C |
| ATOM | 6774 | C39 | POP | B | 902 | −44.850 | 2.078 | −1.367 | 1.00 | 57.75 | C |
| ATOM | 6775 | O | MOO | B | 903 | −53.394 | 1.352 | 13.986 | 1.00 | 83.07 | O |
| ATOM | 6776 | C17 | MOO | B | 903 | −54.212 | 2.037 | 14.578 | 1.00 | 82.46 | C |
| ATOM | 6777 | O1 | MOO | B | 903 | −55.530 | 2.236 | 13.995 | 1.00 | 80.27 | O |
| ATOM | 6778 | C18 | MOO | B | 903 | −56.605 | 2.652 | 14.835 | 1.00 | 91.01 | C |
| ATOM | 6779 | C19 | MOO | B | 903 | −57.570 | 3.542 | 14.060 | 1.00 | 90.68 | C |
| ATOM | 6780 | C20 | MOO | B | 903 | −58.702 | 3.974 | 14.986 | 1.00 | 92.79 | C |
| ATOM | 6781 | O3 | MOO | B | 903 | −58.173 | 4.762 | 16.058 | 1.00 | 76.34 | O |
| ATOM | 6782 | O2 | MOO | B | 903 | −58.100 | 2.826 | 12.937 | 1.00 | 94.58 | O |
| ATOM | 6783 | C16 | MOO | B | 903 | −53.875 | 2.686 | 15.901 | 1.00 | 81.86 | C |
| ATOM | 6784 | C15 | MOO | B | 903 | −53.690 | 1.627 | 16.982 | 1.00 | 82.84 | C |
| ATOM | 6785 | C14 | MOO | B | 903 | −53.752 | 2.260 | 18.368 | 1.00 | 80.59 | C |
| ATOM | 6786 | C13 | MOO | B | 903 | −53.671 | 1.209 | 19.468 | 1.00 | 77.03 | C |
| ATOM | 6787 | C12 | MOO | B | 903 | −52.229 | 0.947 | 19.888 | 1.00 | 76.59 | C |
| ATOM | 6788 | C11 | MOO | B | 903 | −52.149 | −0.280 | 20.790 | 1.00 | 79.82 | C |
| ATOM | 6789 | C10 | MOO | B | 903 | −50.779 | −0.403 | 21.446 | 1.00 | 71.58 | C |
| ATOM | 6790 | C9 | MOO | B | 903 | −50.646 | 0.632 | 22.537 | 1.00 | 64.63 | C |
| ATOM | 6791 | C8 | MOO | B | 903 | −49.554 | 0.658 | 23.297 | 1.00 | 64.85 | C |
| ATOM | 6792 | C7 | MOO | B | 903 | −49.414 | 1.690 | 24.392 | 1.00 | 62.81 | C |
| ATOM | 6793 | C6 | MOO | B | 903 | −48.069 | 1.508 | 25.086 | 1.00 | 51.88 | C |
| ATOM | 6794 | C5 | MOO | B | 903 | −47.905 | 2.489 | 26.241 | 1.00 | 41.51 | C |
| ATOM | 6795 | C4 | MOO | B | 903 | −46.684 | 2.126 | 27.076 | 1.00 | 44.52 | C |
| ATOM | 6796 | C3 | MOO | B | 903 | −46.641 | 2.915 | 28.379 | 1.00 | 45.52 | C |
| ATOM | 6797 | C2 | MOO | B | 903 | −46.234 | 4.363 | 28.141 | 1.00 | 41.79 | C |
| ATOM | 6798 | C1 | MOO | B | 903 | −45.890 | 5.050 | 29.457 | 1.00 | 37.15 | C |
| ATOM | 6799 | C | MOO | B | 903 | −44.792 | 4.306 | 30.186 | 1.00 | 35.98 | C |
| ATOM | 6800 | O | MOO | B | 904 | −50.412 | 35.145 | 19.026 | 1.00 | 75.65 | O |
| ATOM | 6801 | C17 | MOO | B | 904 | −50.517 | 35.070 | 20.239 | 1.00 | 71.48 | C |
| ATOM | 6802 | O1 | MOO | B | 904 | −51.847 | 35.087 | 20.833 | 1.00 | 67.36 | O |
| ATOM | 6803 | C18 | MOO | B | 904 | −52.746 | 36.159 | 20.549 | 1.00 | 67.65 | C |
| ATOM | 6804 | C19 | MOO | B | 904 | −54.042 | 35.973 | 21.333 | 1.00 | 79.49 | C |
| ATOM | 6805 | C20 | MOO | B | 904 | −54.880 | 34.869 | 20.698 | 1.00 | 84.79 | C |
| ATOM | 6806 | O3 | MOO | B | 904 | −55.440 | 35.341 | 19.467 | 1.00 | 71.88 | O |
| ATOM | 6807 | O2 | MOO | B | 904 | −54.793 | 37.193 | 21.309 | 1.00 | 72.99 | O |
| ATOM | 6808 | C16 | MOO | B | 904 | −49.285 | 34.948 | 21.108 | 1.00 | 56.12 | C |
| ATOM | 6809 | C15 | MOO | B | 904 | −48.275 | 36.029 | 20.736 | 1.00 | 54.36 | C |
| ATOM | 6810 | C14 | MOO | B | 904 | −46.968 | 35.861 | 21.506 | 1.00 | 52.14 | C |
| ATOM | 6811 | C13 | MOO | B | 904 | −46.369 | 34.480 | 21.269 | 1.00 | 54.03 | C |
| ATOM | 6812 | C12 | MOO | B | 904 | −44.995 | 34.337 | 21.917 | 1.00 | 49.65 | C |
| ATOM | 6813 | C11 | MOO | B | 904 | −43.952 | 35.183 | 21.196 | 1.00 | 56.32 | C |
| ATOM | 6814 | C10 | MOO | B | 904 | −42.552 | 34.918 | 21.742 | 1.00 | 57.54 | C |
| ATOM | 6815 | C9 | MOO | B | 904 | −41.546 | 35.759 | 20.990 | 1.00 | 57.29 | C |
| ATOM | 6816 | C8 | MOO | B | 904 | −40.242 | 35.603 | 21.208 | 1.00 | 51.69 | C |
| ATOM | 6817 | C7 | MOO | B | 904 | −39.243 | 36.446 | 20.452 | 1.00 | 58.60 | C |
| ATOM | 6818 | C6 | MOO | B | 904 | −37.839 | 35.884 | 20.646 | 1.00 | 61.39 | C |
| ATOM | 6819 | C5 | MOO | B | 904 | −36.828 | 36.606 | 19.761 | 1.00 | 63.94 | C |
| ATOM | 6820 | C4 | MOO | B | 904 | −36.729 | 38.082 | 20.130 | 1.00 | 68.18 | C |
| ATOM | 6821 | C3 | MOO | B | 904 | −35.765 | 38.825 | 19.211 | 1.00 | 61.59 | C |
| ATOM | 6822 | C2 | MOO | B | 904 | −34.360 | 38.239 | 19.285 | 1.00 | 58.37 | C |
| ATOM | 6823 | C1 | MOO | B | 904 | −33.385 | 39.049 | 18.438 | 1.00 | 53.11 | C |
| ATOM | 6824 | C | MOO | B | 904 | −32.004 | 38.431 | 18.451 | 1.00 | 51.32 | C |
| TER | | | | | | | | | | | |

TABLE C

| | | | | | | | | | | | |
|---|---|---|---|---|---|---|---|---|---|---|---|
| ATOM | 6825 | O | HIS | C | 117 | −61.904 | −0.629 | 61.782 | 1.00 | 88.82 | O |
| ATOM | 6826 | N | HIS | C | 117 | −64.566 | 0.819 | 63.498 | 1.00 | 106.92 | N |
| ATOM | 6827 | CA | HIS | C | 117 | −64.113 | 0.173 | 62.272 | 1.00 | 105.42 | C |
| ATOM | 6828 | C | HIS | C | 117 | −62.608 | 0.334 | 62.087 | 1.00 | 93.35 | C |
| ATOM | 6829 | CB | HIS | C | 117 | −64.862 | 0.727 | 61.057 | 1.00 | 113.59 | C |
| ATOM | 6830 | CG | HIS | C | 117 | −66.347 | 0.544 | 61.128 | 1.00 | 125.37 | C |
| ATOM | 6831 | ND1 | HIS | C | 117 | −66.950 | −0.691 | 61.028 | 1.00 | 128.41 | N |
| ATOM | 6832 | CD2 | HIS | C | 117 | −67.350 | 1.441 | 61.286 | 1.00 | 128.73 | C |
| ATOM | 6833 | CE1 | HIS | C | 117 | −68.260 | −0.548 | 61.123 | 1.00 | 130.39 | C |
| ATOM | 6834 | NE2 | HIS | C | 117 | −68.529 | 0.736 | 61.280 | 1.00 | 131.66 | N |
| ATOM | 6835 | N | VAL | C | 118 | −62.121 | 1.556 | 62.274 | 1.00 | 94.66 | N |
| ATOM | 6836 | CA | VAL | C | 118 | −60.699 | 1.845 | 62.128 | 1.00 | 86.42 | C |
| ATOM | 6837 | CB | VAL | C | 118 | −60.394 | 3.327 | 62.410 | 1.00 | 86.70 | C |
| ATOM | 6838 | CG1 | VAL | C | 118 | −58.921 | 3.622 | 62.174 | 1.00 | 89.68 | C |
| ATOM | 6839 | CG2 | VAL | C | 118 | −61.266 | 4.222 | 61.544 | 1.00 | 94.38 | C |
| ATOM | 6840 | C | VAL | C | 118 | −59.935 | 0.980 | 63.124 | 1.00 | 83.90 | C |
| ATOM | 6841 | O | VAL | C | 118 | −58.891 | 0.416 | 62.798 | 1.00 | 77.61 | O |
| ATOM | 6842 | N | ALA | C | 119 | −60.462 | 0.880 | 64.340 | 1.00 | 87.53 | N |
| ATOM | 6843 | CA | ALA | C | 119 | −59.831 | 0.082 | 65.387 | 1.00 | 79.01 | C |
| ATOM | 6844 | CB | ALA | C | 119 | −60.625 | 0.188 | 66.681 | 1.00 | 80.09 | C |
| ATOM | 6845 | C | ALA | C | 119 | −59.655 | −1.391 | 64.987 | 1.00 | 72.26 | C |
| ATOM | 6846 | O | ALA | C | 119 | −58.633 | −2.003 | 65.243 | 1.00 | 76.18 | O |
| ATOM | 6847 | N | ALA | C | 120 | −60.670 | −1.958 | 64.353 | 1.00 | 73.58 | N |
| ATOM | 6848 | CA | ALA | C | 120 | −60.621 | −3.353 | 63.923 | 1.00 | 68.51 | C |
| ATOM | 6849 | CB | ALA | C | 120 | −61.958 | −3.776 | 63.343 | 1.00 | 74.82 | C |
| ATOM | 6850 | C | ALA | C | 120 | −59.502 | −3.583 | 62.907 | 1.00 | 65.72 | C |
| ATOM | 6851 | O | ALA | C | 120 | −58.789 | −4.574 | 62.943 | 1.00 | 66.13 | O |
| ATOM | 6852 | N | ILE | C | 121 | −59.337 | −2.611 | 62.018 | 1.00 | 66.02 | N |
| ATOM | 6853 | CA | ILE | C | 121 | −58.247 | −2.599 | 61.072 | 1.00 | 61.06 | C |
| ATOM | 6854 | CB | ILE | C | 121 | −58.369 | −1.402 | 60.109 | 1.00 | 65.13 | C |
| ATOM | 6855 | CG1 | ILE | C | 121 | −59.723 | −1.440 | 59.397 | 1.00 | 63.95 | C |
| ATOM | 6856 | CD1 | ILE | C | 121 | −59.986 | −0.236 | 58.522 | 1.00 | 62.49 | C |
| ATOM | 6857 | CG2 | ILE | C | 121 | −57.236 | −1.403 | 59.094 | 1.00 | 60.39 | C |
| ATOM | 6858 | C | ILE | C | 121 | −56.907 | −2.567 | 61.792 | 1.00 | 69.16 | C |
| ATOM | 6859 | O | ILE | C | 121 | −56.002 | −3.288 | 61.442 | 1.00 | 67.86 | O |
| ATOM | 6860 | N | ILE | C | 122 | −56.766 | −1.730 | 62.824 | 1.00 | 73.00 | N |
| ATOM | 6861 | CA | ILE | C | 122 | −55.488 | −1.674 | 63.539 | 1.00 | 65.50 | C |
| ATOM | 6862 | CB | ILE | C | 122 | −55.547 | −0.643 | 64.687 | 1.00 | 72.39 | C |
| ATOM | 6863 | CG1 | ILE | C | 122 | −55.758 | 0.768 | 64.133 | 1.00 | 74.61 | C |
| ATOM | 6864 | CD1 | ILE | C | 122 | −55.831 | 1.836 | 65.195 | 1.00 | 83.26 | C |
| ATOM | 6865 | CG2 | ILE | C | 122 | −54.285 | −0.699 | 65.525 | 1.00 | 71.82 | C |
| ATOM | 6866 | C | ILE | C | 122 | −55.142 | −3.025 | 64.100 | 1.00 | 59.76 | C |
| ATOM | 6867 | O | ILE | C | 122 | −54.024 | −3.478 | 64.010 | 1.00 | 60.14 | O |
| ATOM | 6868 | N | ASN | C | 123 | −56.125 | −3.653 | 64.711 | 1.00 | 62.71 | N |
| ATOM | 6869 | CA | ASN | C | 123 | −55.923 | −4.939 | 65.341 | 1.00 | 59.94 | C |
| ATOM | 6870 | CB | ASN | C | 123 | −57.225 | −5.366 | 66.025 | 1.00 | 61.06 | C |
| ATOM | 6871 | CG | ASN | C | 123 | −56.994 | −6.096 | 67.332 | 1.00 | 64.66 | C |
| ATOM | 6872 | OD1 | ASN | C | 123 | −55.926 | −6.660 | 67.561 | 1.00 | 68.11 | O |
| ATOM | 6873 | ND2 | ASN | C | 123 | −58.004 | −6.092 | 68.197 | 1.00 | 64.04 | N |
| ATOM | 6874 | C | ASN | C | 123 | −55.509 | −6.022 | 64.341 | 1.00 | 58.30 | C |
| ATOM | 6875 | O | ASN | C | 123 | −54.568 | −6.768 | 64.534 | 1.00 | 60.30 | O |
| ATOM | 6876 | N | TYR | C | 124 | −56.203 | −6.072 | 63.234 | 1.00 | 61.30 | N |
| ATOM | 6877 | CA | TYR | C | 124 | −55.870 | −7.002 | 62.184 | 1.00 | 62.10 | C |
| ATOM | 6878 | CB | TYR | C | 124 | −56.907 | −6.881 | 61.060 | 1.00 | 62.05 | C |
| ATOM | 6879 | CG | TYR | C | 124 | −56.843 | −7.950 | 59.990 | 1.00 | 61.15 | C |
| ATOM | 6880 | CD1 | TYR | C | 124 | −57.412 | −9.203 | 60.196 | 1.00 | 63.01 | C |
| ATOM | 6881 | CE1 | TYR | C | 124 | −57.368 | −10.178 | 59.215 | 1.00 | 64.24 | C |
| ATOM | 6882 | CZ | TYR | C | 124 | −56.759 | −9.903 | 58.009 | 1.00 | 74.52 | C |
| ATOM | 6883 | OH | TYR | C | 124 | −56.714 | −10.870 | 57.029 | 1.00 | 77.19 | O |
| ATOM | 6884 | CE2 | TYR | C | 124 | −56.195 | −8.665 | 57.777 | 1.00 | 76.41 | C |
| ATOM | 6885 | CD2 | TYR | C | 124 | −56.244 | −7.695 | 58.763 | 1.00 | 61.05 | C |
| ATOM | 6886 | C | TYR | C | 124 | −54.455 | −6.790 | 61.622 | 1.00 | 63.25 | C |
| ATOM | 6887 | O | TYR | C | 124 | −53.697 | −7.718 | 61.398 | 1.00 | 59.95 | O |
| ATOM | 6888 | N | LEU | C | 125 | −54.161 | −5.529 | 61.347 | 1.00 | 66.23 | N |
| ATOM | 6889 | CA | LEU | C | 125 | −52.911 | −5.098 | 60.760 | 1.00 | 65.00 | C |
| ATOM | 6890 | CB | LEU | C | 125 | −52.982 | −3.608 | 60.417 | 1.00 | 63.52 | C |
| ATOM | 6891 | CG | LEU | C | 125 | −51.959 | −3.083 | 59.411 | 1.00 | 67.29 | C |
| ATOM | 6892 | CD1 | LEU | C | 125 | −52.457 | −3.288 | 57.986 | 1.00 | 65.48 | C |
| ATOM | 6893 | CD2 | LEU | C | 125 | −51.648 | −1.620 | 59.678 | 1.00 | 65.64 | C |
| ATOM | 6894 | C | LEU | C | 125 | −51.716 | −5.366 | 61.664 | 1.00 | 61.90 | C |
| ATOM | 6895 | O | LEU | C | 125 | −50.683 | −5.863 | 61.256 | 1.00 | 60.06 | O |
| ATOM | 6896 | N | GLY | C | 126 | −51.900 | −5.048 | 62.926 | 1.00 | 59.40 | N |
| ATOM | 6897 | CA | GLY | C | 126 | −50.947 | −5.271 | 63.947 | 1.00 | 57.85 | C |
| ATOM | 6898 | C | GLY | C | 126 | −50.583 | −6.688 | 64.097 | 1.00 | 56.28 | C |
| ATOM | 6899 | O | GLY | C | 126 | −49.442 | −7.032 | 64.290 | 1.00 | 52.64 | O |
| ATOM | 6900 | N | HIS | C | 127 | −51.572 | −7.551 | 63.989 | 1.00 | 58.60 | N |
| ATOM | 6901 | CA | HIS | C | 127 | −51.342 | −8.982 | 64.062 | 1.00 | 52.61 | C |
| ATOM | 6902 | CB | HIS | C | 127 | −52.649 | −9.760 | 64.202 | 1.00 | 52.16 | C |
| ATOM | 6903 | CG | HIS | C | 127 | −53.102 | −9.889 | 65.623 | 1.00 | 47.57 | C |
| ATOM | 6904 | ND1 | HIS | C | 127 | −53.940 | −8.975 | 66.223 | 1.00 | 47.18 | N |
| ATOM | 6905 | CE1 | HIS | C | 127 | −54.145 | −9.330 | 67.480 | 1.00 | 47.17 | C |
| ATOM | 6906 | NE2 | HIS | C | 127 | −53.458 | −10.431 | 67.720 | 1.00 | 48.36 | N |
| ATOM | 6907 | CD2 | HIS | C | 127 | −52.789 | −10.798 | 66.576 | 1.00 | 50.34 | C |
| ATOM | 6908 | C | HIS | C | 127 | −50.472 | −9.486 | 62.913 | 1.00 | 51.88 | C |
| ATOM | 6909 | O | HIS | C | 127 | −49.618 | −10.322 | 63.112 | 1.00 | 51.70 | O |
| ATOM | 6910 | N | CYS | C | 128 | −50.702 | −8.980 | 61.695 | 1.00 | 55.40 | N |
| ATOM | 6911 | CA | CYS | C | 128 | −49.882 | −9.383 | 60.554 | 1.00 | 52.38 | C |
| ATOM | 6912 | CB | CYS | C | 128 | −50.486 | −8.852 | 59.252 | 1.00 | 48.92 | C |
| ATOM | 6913 | SG | CYS | C | 128 | −52.223 | −9.282 | 59.024 | 1.00 | 67.08 | S |
| ATOM | 6914 | C | CYS | C | 128 | −48.430 | −8.923 | 60.678 | 1.00 | 49.32 | C |
| ATOM | 6915 | O | CYS | C | 128 | −47.512 | −9.646 | 60.342 | 1.00 | 44.30 | O |
| ATOM | 6916 | N | ILE | C | 129 | −48.226 | −7.719 | 61.201 | 1.00 | 49.46 | N |
| ATOM | 6917 | CA | ILE | C | 129 | −46.878 | −7.209 | 61.427 | 1.00 | 48.21 | C |
| ATOM | 6918 | CB | ILE | C | 129 | −46.918 | −5.741 | 61.893 | 1.00 | 52.02 | C |
| ATOM | 6919 | CG1 | ILE | C | 129 | −47.556 | −4.858 | 60.823 | 1.00 | 54.78 | C |
| ATOM | 6920 | CD1 | ILE | C | 129 | −47.683 | −3.405 | 61.236 | 1.00 | 56.60 | C |
| ATOM | 6921 | CG2 | ILE | C | 129 | −45.519 | −5.238 | 62.233 | 1.00 | 55.28 | C |
| ATOM | 6922 | C | ILE | C | 129 | −46.140 | −8.025 | 62.467 | 1.00 | 53.52 | C |
| ATOM | 6923 | O | ILE | C | 129 | −44.978 | −8.341 | 62.315 | 1.00 | 52.84 | O |
| ATOM | 6924 | N | SER | C | 130 | −46.830 | −8.345 | 63.550 | 1.00 | 54.89 | N |
| ATOM | 6925 | CA | SER | C | 130 | −46.257 | −9.117 | 64.621 | 1.00 | 49.79 | C |
| ATOM | 6926 | CB | SER | C | 130 | −47.222 | −9.199 | 65.806 | 1.00 | 48.47 | C |
| ATOM | 6927 | OG | SER | C | 130 | −47.474 | −7.920 | 66.351 | 1.00 | 55.64 | O |
| ATOM | 6928 | C | SER | C | 130 | −45.877 | −10.506 | 64.190 | 1.00 | 46.89 | C |
| ATOM | 6929 | O | SER | C | 130 | −44.856 | −11.026 | 64.568 | 1.00 | 47.65 | O |
| ATOM | 6930 | N | LEU | C | 131 | −46.745 | −11.126 | 63.434 | 1.00 | 45.81 | N |
| ATOM | 6931 | CA | LEU | C | 131 | −46.509 | −12.459 | 62.939 | 1.00 | 46.35 | C |
| ATOM | 6932 | CB | LEU | C | 131 | −47.736 | −12.990 | 62.195 | 1.00 | 48.31 | C |
| ATOM | 6933 | CG | LEU | C | 131 | −47.580 | −14.420 | 61.675 | 1.00 | 43.11 | C |
| ATOM | 6934 | CD1 | LEU | C | 131 | −47.414 | −15.391 | 62.831 | 1.00 | 39.30 | C |
| ATOM | 6935 | CD2 | LEU | C | 131 | −48.756 | −14.813 | 60.799 | 1.00 | 42.57 | C |
| ATOM | 6936 | C | LEU | C | 131 | −45.307 | −12.534 | 62.054 | 1.00 | 48.19 | C |
| ATOM | 6937 | O | LEU | C | 131 | −44.445 | −13.359 | 62.239 | 1.00 | 48.99 | O |
| ATOM | 6938 | N | VAL | C | 132 | −45.257 | −11.631 | 61.089 | 1.00 | 47.74 | N |
| ATOM | 6939 | CA | VAL | C | 132 | −44.129 | −11.530 | 60.194 | 1.00 | 46.80 | C |
| ATOM | 6940 | CB | VAL | C | 132 | −44.340 | −10.427 | 59.135 | 1.00 | 43.40 | C |
| ATOM | 6941 | CG1 | VAL | C | 132 | −43.064 | −10.202 | 58.336 | 1.00 | 39.57 | C |
| ATOM | 6942 | CG2 | VAL | C | 132 | −45.488 | −10.795 | 58.211 | 1.00 | 41.01 | C |
| ATOM | 6943 | C | VAL | C | 132 | −42.844 | −11.251 | 60.969 | 1.00 | 46.53 | C |
| ATOM | 6944 | O | VAL | C | 132 | −41.838 | −11.885 | 60.755 | 1.00 | 47.96 | O |
| ATOM | 6945 | N | ALA | C | 133 | −42.895 | −10.317 | 61.904 | 1.00 | 42.35 | N |
| ATOM | 6946 | CA | ALA | C | 133 | −41.746 | −9.991 | 62.715 | 1.00 | 40.29 | C |
| ATOM | 6947 | CB | ALA | C | 133 | −42.065 | −8.815 | 63.634 | 1.00 | 40.13 | C |
| ATOM | 6948 | C | ALA | C | 133 | −41.221 | −11.164 | 63.525 | 1.00 | 42.03 | C |
| ATOM | 6949 | O | ALA | C | 133 | −40.052 | −11.464 | 63.498 | 1.00 | 44.80 | O |
| ATOM | 6950 | N | LEU | C | 134 | −42.103 | −11.855 | 64.215 | 1.00 | 42.87 | N |
| ATOM | 6951 | CA | LEU | C | 134 | −41.724 | −12.989 | 65.008 | 1.00 | 40.42 | C |
| ATOM | 6952 | CB | LEU | C | 134 | −42.938 | −13.550 | 65.745 | 1.00 | 40.42 | C |
| ATOM | 6953 | CG | LEU | C | 134 | −43.441 | −12.814 | 66.981 | 1.00 | 43.94 | C |
| ATOM | 6954 | CD1 | LEU | C | 134 | −44.867 | −13.241 | 67.281 | 1.00 | 43.58 | C |
| ATOM | 6955 | CD2 | LEU | C | 134 | −42.535 | −13.107 | 68.165 | 1.00 | 44.86 | C |
| ATOM | 6956 | C | LEU | C | 134 | −41.129 | −14.071 | 64.149 | 1.00 | 42.61 | C |
| ATOM | 6957 | O | LEU | C | 134 | −40.156 | −14.687 | 64.500 | 1.00 | 44.96 | O |
| ATOM | 6958 | N | LEU | C | 135 | −41.750 | −14.314 | 63.015 | 1.00 | 42.23 | N |
| ATOM | 6959 | CA | LEU | C | 135 | −41.316 | −15.347 | 62.106 | 1.00 | 42.79 | C |
| ATOM | 6960 | CB | LEU | C | 135 | −42.308 | −15.520 | 60.949 | 1.00 | 40.95 | C |
| ATOM | 6961 | CG | LEU | C | 135 | −43.390 | −16.585 | 61.157 | 1.00 | 38.06 | C |
| ATOM | 6962 | CD1 | LEU | C | 135 | −44.481 | −16.469 | 60.112 | 1.00 | 42.80 | C |
| ATOM | 6963 | CD2 | LEU | C | 135 | −42.778 | −17.973 | 61.116 | 1.00 | 43.27 | C |
| ATOM | 6964 | C | LEU | C | 135 | −39.940 | −15.065 | 61.577 | 1.00 | 45.04 | C |
| ATOM | 6965 | O | LEU | C | 135 | −39.115 | −15.944 | 61.508 | 1.00 | 41.85 | O |
| ATOM | 6966 | N | VAL | C | 136 | −39.693 | −13.814 | 61.198 | 1.00 | 48.03 | N |
| ATOM | 6967 | CA | VAL | C | 136 | −38.373 | −13.399 | 60.754 | 1.00 | 45.26 | C |
| ATOM | 6968 | CB | VAL | C | 136 | −38.356 | −11.922 | 60.289 | 1.00 | 36.17 | C |
| ATOM | 6969 | CG1 | VAL | C | 136 | −36.929 | −11.433 | 60.089 | 1.00 | 34.99 | C |
| ATOM | 6970 | CG2 | VAL | C | 136 | −39.142 | −11.768 | 59.006 | 1.00 | 41.78 | C |
| ATOM | 6971 | C | VAL | C | 136 | −37.327 | −13.607 | 61.827 | 1.00 | 44.08 | C |
| ATOM | 6972 | O | VAL | C | 136 | −36.273 | −14.150 | 61.582 | 1.00 | 43.22 | O |
| ATOM | 6973 | N | ALA | C | 137 | −37.654 | −13.233 | 63.048 | 1.00 | 40.24 | N |
| ATOM | 6974 | CA | ALA | C | 137 | −36.739 | −13.410 | 64.142 | 1.00 | 43.28 | C |
| ATOM | 6975 | CB | ALA | C | 137 | −37.323 | −12.818 | 65.417 | 1.00 | 46.63 | C |
| ATOM | 6976 | C | ALA | C | 137 | −36.399 | −14.858 | 64.346 | 1.00 | 46.70 | C |
| ATOM | 6977 | O | ALA | C | 137 | −35.261 | −15.232 | 64.503 | 1.00 | 43.12 | O |
| ATOM | 6978 | N | PHE | C | 138 | −37.415 | −15.678 | 64.298 | 1.00 | 53.30 | N |
| ATOM | 6979 | CA | PHE | C | 138 | −37.283 | −17.115 | 64.425 | 1.00 | 50.99 | C |
| ATOM | 6980 | CB | PHE | C | 138 | −38.677 | −17.747 | 64.281 | 1.00 | 50.31 | C |
| ATOM | 6981 | CG | PHE | C | 138 | −38.715 | −19.225 | 64.538 | 1.00 | 54.30 | C |
| ATOM | 6982 | CD1 | PHE | C | 138 | −38.414 | −19.737 | 65.788 | 1.00 | 57.97 | C |
| ATOM | 6983 | CE1 | PHE | C | 138 | −38.457 | −21.101 | 66.018 | 1.00 | 60.16 | C |
| ATOM | 6984 | CZ | PHE | C | 138 | −38.815 | −21.964 | 64.999 | 1.00 | 52.78 | C |
| ATOM | 6985 | CE2 | PHE | C | 138 | −39.126 | −21.464 | 63.756 | 1.00 | 52.82 | C |
| ATOM | 6986 | CD2 | PHE | C | 138 | −39.081 | −20.103 | 63.531 | 1.00 | 54.88 | C |
| ATOM | 6987 | C | PHE | C | 138 | −36.314 | −17.696 | 63.396 | 1.00 | 47.41 | C |
| ATOM | 6988 | O | PHE | C | 138 | −35.420 | −18.425 | 63.747 | 1.00 | 47.34 | O |
| ATOM | 6989 | N | VAL | C | 139 | −36.472 | −17.332 | 62.130 | 1.00 | 45.33 | N |
| ATOM | 6990 | CA | VAL | C | 139 | −35.588 | −17.796 | 61.081 | 1.00 | 44.10 | C |
| ATOM | 6991 | CB | VAL | C | 139 | −36.073 | −17.308 | 59.693 | 1.00 | 37.65 | C |
| ATOM | 6992 | CG1 | VAL | C | 139 | −34.987 | −17.457 | 58.642 | 1.00 | 38.35 | C |
| ATOM | 6993 | CG2 | VAL | C | 139 | −37.316 | −18.069 | 59.282 | 1.00 | 46.94 | C |
| ATOM | 6994 | C | VAL | C | 139 | −34.154 | −17.365 | 61.322 | 1.00 | 48.02 | C |
| ATOM | 6995 | O | VAL | C | 139 | −33.233 | −18.141 | 61.167 | 1.00 | 49.37 | O |
| ATOM | 6996 | N | LEU | C | 140 | −33.973 | −16.125 | 61.750 | 1.00 | 46.07 | N |
| ATOM | 6997 | CA | LEU | C | 140 | −32.650 | −15.625 | 62.091 | 1.00 | 46.14 | C |
| ATOM | 6998 | CB | LEU | C | 140 | −32.709 | −14.139 | 62.454 | 1.00 | 39.41 | C |
| ATOM | 6999 | CG | LEU | C | 140 | −33.137 | −13.231 | 61.301 | 1.00 | 32.78 | C |
| ATOM | 7000 | CD1 | LEU | C | 140 | −33.081 | −11.774 | 61.712 | 1.00 | 33.33 | C |
| ATOM | 7001 | CD2 | LEU | C | 140 | −32.270 | −13.482 | 60.081 | 1.00 | 40.64 | C |
| ATOM | 7002 | C | LEU | C | 140 | −31.956 | −16.433 | 63.198 | 1.00 | 51.70 | C |
| ATOM | 7003 | O | LEU | C | 140 | −30.778 | −16.750 | 63.112 | 1.00 | 58.17 | O |
| ATOM | 7004 | N | PHE | C | 141 | −32.696 | −16.782 | 64.244 | 1.00 | 47.58 | N |
| ATOM | 7005 | CA | PHE | C | 141 | −32.141 | −17.604 | 65.303 | 1.00 | 49.51 | C |
| ATOM | 7006 | CB | PHE | C | 141 | −33.123 | −17.722 | 66.474 | 1.00 | 49.24 | C |
| ATOM | 7007 | CG | PHE | C | 141 | −33.035 | −16.591 | 67.464 | 1.00 | 53.36 | C |
| ATOM | 7008 | CD1 | PHE | C | 141 | −32.155 | −16.657 | 68.530 | 1.00 | 63.03 | C |
| ATOM | 7009 | CE1 | PHE | C | 141 | −32.072 | −15.624 | 69.447 | 1.00 | 61.40 | C |
| ATOM | 7010 | CZ | PHE | C | 141 | −32.873 | −14.512 | 69.309 | 1.00 | 59.68 | C |
| ATOM | 7011 | CE2 | PHE | C | 141 | −33.757 | −14.432 | 68.255 | 1.00 | 61.84 | C |
| ATOM | 7012 | CD2 | PHE | C | 141 | −33.837 | −15.470 | 67.338 | 1.00 | 55.82 | C |
| ATOM | 7013 | C | PHE | C | 141 | −31.779 | −18.993 | 64.785 | 1.00 | 54.32 | C |
| ATOM | 7014 | O | PHE | C | 141 | −30.737 | −19.515 | 65.110 | 1.00 | 58.99 | O |
| ATOM | 7015 | N | LEU | C | 142 | −32.598 | −19.537 | 63.894 | 1.00 | 51.62 | N |
| ATOM | 7016 | CA | LEU | C | 142 | −32.268 | −20.763 | 63.177 | 1.00 | 50.75 | C |
| ATOM | 7017 | CB | LEU | C | 142 | −33.428 | −21.212 | 62.281 | 1.00 | 57.75 | C |
| ATOM | 7018 | CG | LEU | C | 142 | −34.789 | −21.468 | 62.951 | 1.00 | 59.25 | C |
| ATOM | 7019 | CD1 | LEU | C | 142 | −35.748 | −22.170 | 61.989 | 1.00 | 56.34 | C |
| ATOM | 7020 | CD2 | LEU | C | 142 | −34.660 | −22.248 | 64.255 | 1.00 | 48.48 | C |
| ATOM | 7021 | C | LEU | C | 142 | −30.951 | −20.683 | 62.380 | 1.00 | 57.20 | C |
| ATOM | 7022 | O | LEU | C | 142 | −30.197 | −21.638 | 62.322 | 1.00 | 65.14 | O |
| ATOM | 7023 | N | ARG | C | 143 | −30.707 | −19.567 | 61.699 | 1.00 | 61.69 | N |
| ATOM | 7024 | CA | ARG | C | 143 | −29.477 | −19.401 | 60.911 | 1.00 | 66.85 | C |
| ATOM | 7025 | CB | ARG | C | 143 | −29.627 | −18.131 | 60.065 | 1.00 | 75.36 | C |
| ATOM | 7026 | CG | ARG | C | 143 | −28.428 | −17.750 | 59.219 | 1.00 | 102.03 | C |
| ATOM | 7027 | CD | ARG | C | 143 | −28.663 | −16.406 | 58.547 | 1.00 | 103.47 | C |
| ATOM | 7028 | NE | ARG | C | 143 | −27.545 | −16.007 | 57.696 | 1.00 | 138.00 | N |
| ATOM | 7029 | CZ | ARG | C | 143 | −26.480 | −15.333 | 58.122 | 1.00 | 138.95 | C |
| ATOM | 7030 | NH1 | ARG | C | 143 | −26.378 | −14.983 | 59.400 | 1.00 | 129.57 | N |
| ATOM | 7031 | NH2 | ARG | C | 143 | −25.515 | −15.010 | 57.271 | 1.00 | 134.46 | N |
| ATOM | 7032 | C | ARG | C | 143 | −28.156 | −19.321 | 61.756 | 1.00 | 72.24 | C |
| ATOM | 7033 | O | ARG | C | 143 | −27.109 | −19.815 | 61.363 | 1.00 | 74.10 | O |
| ATOM | 7034 | N | ALA | C | 144 | −28.191 | −18.643 | 62.887 | 1.00 | 76.11 | N |
| ATOM | 7035 | CA | ALA | C | 144 | −26.988 | −18.449 | 63.713 | 1.00 | 80.34 | C |
| ATOM | 7036 | CB | ALA | C | 144 | −27.243 | −17.388 | 64.772 | 1.00 | 72.74 | C |
| ATOM | 7037 | C | ALA | C | 144 | −26.418 | −19.752 | 64.366 | 1.00 | 81.00 | C |
| ATOM | 7038 | O | ALA | C | 144 | −27.163 | −20.628 | 64.792 | 1.00 | 78.94 | O |
| ATOM | 7039 | O | ARG | C | 145 | −24.680 | −21.913 | 67.435 | 1.00 | 87.14 | O |
| ATOM | 7040 | N | ARG | C | 145 | −25.071 | −19.819 | 64.495 | 1.00 | 83.45 | N |
| ATOM | 7041 | CA | ARG | C | 145 | −24.379 | −20.905 | 65.222 | 1.00 | 86.96 | C |
| ATOM | 7042 | C | ARG | C | 145 | −24.742 | −20.908 | 66.715 | 1.00 | 86.38 | C |
| ATOM | 7043 | CB | ARG | C | 145 | −22.863 | −20.768 | 65.081 | 1.00 | 104.57 | C |
| ATOM | 7044 | CG | ARG | C | 145 | −22.332 | −20.743 | 63.658 | 1.00 | 110.29 | C |
| ATOM | 7045 | CD | ARG | C | 145 | −20.826 | −20.525 | 63.679 | 1.00 | 124.94 | C |
| ATOM | 7046 | NE | ARG | C | 145 | −20.235 | −20.479 | 62.344 | 1.00 | 135.24 | N |
| ATOM | 7047 | CZ | ARG | C | 145 | −18.939 | −20.291 | 62.112 | 1.00 | 134.41 | C |
| ATOM | 7048 | NH1 | ARG | C | 145 | −18.101 | −20.133 | 63.128 | 1.00 | 137.82 | N |
| ATOM | 7049 | NH2 | ARG | C | 145 | −18.479 | −20.262 | 60.868 | 1.00 | 127.98 | N |
| ATOM | 7050 | O | SER | C | 146 | −26.966 | −20.609 | 70.139 | 1.00 | 80.09 | O |
| ATOM | 7051 | N | SER | C | 146 | −25.146 | −19.722 | 67.174 | 1.00 | 79.56 | N |
| ATOM | 7052 | CA | SER | C | 146 | −25.553 | −19.489 | 68.548 | 1.00 | 75.54 | C |
| ATOM | 7053 | C | SER | C | 146 | −26.719 | −20.398 | 68.964 | 1.00 | 79.70 | C |
| ATOM | 7054 | CB | SER | C | 146 | −25.858 | −18.009 | 68.808 | 1.00 | 66.78 | C |
| ATOM | 7055 | OG | SER | C | 146 | −26.773 | −17.495 | 67.863 | 1.00 | 86.71 | O |
| ATOM | 7056 | N | ILE | C | 147 | −27.421 | −20.954 | 67.965 | 1.00 | 81.51 | N |
| ATOM | 7057 | CA | ILE | C | 147 | −28.553 | −21.848 | 68.188 | 1.00 | 81.75 | C |
| ATOM | 7058 | CB | ILE | C | 147 | −29.342 | −22.210 | 66.915 | 1.00 | 74.49 | C |
| ATOM | 7059 | CG1 | ILE | C | 147 | −30.704 | −22.776 | 67.320 | 1.00 | 71.68 | C |
| ATOM | 7060 | CD1 | ILE | C | 147 | −31.748 | −22.696 | 66.241 | 1.00 | 77.66 | C |
| ATOM | 7061 | CG2 | ILE | C | 147 | −28.579 | −23.197 | 66.043 | 1.00 | 71.26 | C |
| ATOM | 7062 | C | ILE | C | 147 | −28.129 | −23.074 | 68.964 | 1.00 | 82.31 | C |
| ATOM | 7063 | O | ILE | C | 147 | −28.851 | −23.579 | 69.803 | 1.00 | 79.85 | O |
| ATOM | 7064 | N | ARG | C | 148 | −26.924 | −23.549 | 68.692 | 1.00 | 87.96 | N |
| ATOM | 7065 | CA | ARG | C | 148 | −26.386 | −24.684 | 69.429 | 1.00 | 91.96 | C |
| ATOM | 7066 | C | ARG | C | 148 | −26.233 | −24.351 | 70.949 | 1.00 | 84.41 | C |
| ATOM | 7067 | O | ARG | C | 148 | −26.337 | −25.230 | 71.800 | 1.00 | 86.40 | O |
| ATOM | 7068 | CB | ARG | C | 148 | −25.030 | −25.101 | 68.865 | 1.00 | 96.49 | C |
| ATOM | 7069 | CG | ARG | C | 148 | −23.898 | −24.162 | 69.210 | 1.00 | 103.54 | C |
| ATOM | 7070 | CD | ARG | C | 148 | −22.599 | −24.600 | 68.576 | 1.00 | 112.63 | C |
| ATOM | 7071 | NE | ARG | C | 148 | −21.706 | −23.463 | 68.400 | 1.00 | 121.35 | N |
| ATOM | 7072 | CZ | ARG | C | 148 | −20.452 | −23.548 | 67.969 | 1.00 | 122.81 | C |
| ATOM | 7073 | NH1 | ARG | C | 148 | −19.916 | −24.729 | 67.675 | 1.00 | 123.61 | N |
| ATOM | 7074 | NH2 | ARG | C | 148 | −19.732 | −22.443 | 67.832 | 1.00 | 113.27 | N |
| ATOM | 7075 | N | CYS | C | 149 | −26.068 | −23.062 | 71.294 | 1.00 | 80.38 | N |
| ATOM | 7076 | CA | CYS | C | 149 | −26.150 | −22.697 | 72.745 | 1.00 | 76.73 | C |
| ATOM | 7077 | CB | CYS | C | 149 | −25.531 | −21.318 | 72.999 | 1.00 | 67.19 | C |
| ATOM | 7078 | SG | CYS | C | 149 | −23.759 | −21.201 | 72.660 | 1.00 | 69.07 | S |
| ATOM | 7079 | C | CYS | C | 149 | −27.599 | −22.742 | 73.322 | 1.00 | 67.22 | C |
| ATOM | 7080 | O | CYS | C | 149 | −28.554 | −22.299 | 72.701 | 1.00 | 66.46 | O |
| ATOM | 7081 | N | LEU | C | 150 | −27.697 | −23.169 | 74.598 | 1.00 | 67.70 | N |
| ATOM | 7082 | CA | LEU | C | 150 | −28.936 | −23.151 | 75.409 | 1.00 | 60.87 | C |
| ATOM | 7083 | CB | LEU | C | 150 | −28.649 | −23.623 | 76.839 | 1.00 | 66.86 | C |
| ATOM | 7084 | CG | LEU | C | 150 | −29.873 | −23.903 | 77.717 | 1.00 | 66.66 | C |
| ATOM | 7085 | CD1 | LEU | C | 150 | −30.863 | −24.802 | 76.988 | 1.00 | 71.51 | C |
| ATOM | 7086 | CD2 | LEU | C | 150 | −29.458 | −24.529 | 79.036 | 1.00 | 67.00 | C |
| ATOM | 7087 | C | LEU | C | 150 | −29.737 | −21.843 | 75.431 | 1.00 | 60.29 | C |
| ATOM | 7088 | O | LEU | C | 150 | −30.949 | −21.863 | 75.501 | 1.00 | 62.53 | O |
| ATOM | 7089 | N | ARG | C | 151 | −29.051 | −20.699 | 75.421 | 1.00 | 63.92 | N |
| ATOM | 7090 | CA | ARG | C | 151 | −29.710 | −19.391 | 75.455 | 1.00 | 61.29 | C |
| ATOM | 7091 | CB | ARG | C | 151 | −28.642 | −18.296 | 75.427 | 1.00 | 68.67 | C |
| ATOM | 7092 | CG | ARG | C | 151 | −29.177 | −16.886 | 75.514 | 1.00 | 78.81 | C |
| ATOM | 7093 | CD | ARG | C | 151 | −28.066 | −15.843 | 75.347 | 1.00 | 83.65 | C |
| ATOM | 7094 | NE | ARG | C | 151 | −27.442 | −15.881 | 74.020 | 1.00 | 100.33 | N |
| ATOM | 7095 | CZ | ARG | C | 151 | −27.923 | −15.260 | 72.943 | 1.00 | 105.76 | C |
| ATOM | 7096 | NH1 | ARG | C | 151 | −29.045 | −14.552 | 73.024 | 1.00 | 92.69 | N |
| ATOM | 7097 | NH2 | ARG | C | 151 | −27.290 | −15.349 | 71.777 | 1.00 | 101.01 | N |
| ATOM | 7098 | C | ARG | C | 151 | −30.667 | −19.216 | 74.300 | 1.00 | 59.08 | C |
| ATOM | 7099 | O | ARG | C | 151 | −31.800 | −18.848 | 74.473 | 1.00 | 57.23 | O |
| ATOM | 7100 | N | ASN | C | 152 | −30.137 | −19.403 | 73.110 | 1.00 | 62.93 | N |
| ATOM | 7101 | CA | ASN | C | 152 | −30.868 | −19.194 | 71.891 | 1.00 | 57.68 | C |
| ATOM | 7102 | CB | ASN | C | 152 | −29.907 | −19.186 | 70.702 | 1.00 | 69.72 | C |
| ATOM | 7103 | CG | ASN | C | 152 | −28.868 | −18.092 | 70.801 | 1.00 | 72.11 | C |
| ATOM | 7104 | OD1 | ASN | C | 152 | −27.910 | −18.197 | 71.568 | 1.00 | 71.95 | O |
| ATOM | 7105 | ND2 | ASN | C | 152 | −29.045 | −17.035 | 70.015 | 1.00 | 77.69 | N |
| ATOM | 7106 | C | ASN | C | 152 | −31.927 | −20.252 | 71.659 | 1.00 | 55.89 | C |
| ATOM | 7107 | O | ASN | C | 152 | −32.934 | −19.995 | 71.032 | 1.00 | 52.88 | O |
| ATOM | 7108 | N | ILE | C | 153 | −31.709 | −21.452 | 72.200 | 1.00 | 55.10 | N |
| ATOM | 7109 | CA | ILE | C | 153 | −32.742 | −22.494 | 72.159 | 1.00 | 57.67 | C |
| ATOM | 7110 | CB | ILE | C | 153 | −32.254 | −23.802 | 72.792 | 1.00 | 56.86 | C |
| ATOM | 7111 | CG1 | ILE | C | 153 | −31.186 | −24.441 | 71.905 | 1.00 | 64.90 | C |
| ATOM | 7112 | CD1 | ILE | C | 153 | −30.669 | −25.765 | 72.429 | 1.00 | 76.77 | C |
| ATOM | 7113 | CG2 | ILE | C | 153 | −33.416 | −24.764 | 72.996 | 1.00 | 58.75 | C |
| ATOM | 7114 | C | ILE | C | 153 | −34.010 | −22.029 | 72.866 | 1.00 | 55.89 | C |
| ATOM | 7115 | O | ILE | C | 153 | −35.094 | −22.059 | 72.320 | 1.00 | 50.70 | O |
| ATOM | 7116 | N | ILE | C | 154 | −33.839 | −21.513 | 74.068 | 1.00 | 52.62 | N |
| ATOM | 7117 | CA | ILE | C | 154 | −34.928 | −20.932 | 74.803 | 1.00 | 47.90 | C |
| ATOM | 7118 | CB | ILE | C | 154 | −34.482 | −20.505 | 76.212 | 1.00 | 47.63 | C |
| ATOM | 7119 | CG1 | ILE | C | 154 | −33.993 | −21.727 | 76.990 | 1.00 | 46.37 | C |
| ATOM | 7120 | CD1 | ILE | C | 154 | −33.431 | −21.399 | 78.354 | 1.00 | 54.33 | C |
| ATOM | 7121 | CG2 | ILE | C | 154 | −35.618 | −19.816 | 76.956 | 1.00 | 47.89 | C |
| ATOM | 7122 | C | ILE | C | 154 | −35.523 | −19.750 | 74.063 | 1.00 | 42.76 | C |
| ATOM | 7123 | O | ILE | C | 154 | −36.710 | −19.632 | 73.951 | 1.00 | 49.43 | O |
| ATOM | 7124 | N | HIS | C | 155 | −34.719 | −18.878 | 73.525 | 1.00 | 48.13 | N |
| ATOM | 7125 | CA | HIS | C | 155 | −35.272 | −17.755 | 72.800 | 1.00 | 55.40 | C |
| ATOM | 7126 | CB | HIS | C | 155 | −34.157 | −16.806 | 72.350 | 1.00 | 61.34 | C |
| ATOM | 7127 | CG | HIS | C | 155 | −33.562 | −16.008 | 73.467 | 1.00 | 58.49 | C |
| ATOM | 7128 | ND1 | HIS | C | 155 | −32.306 | −16.256 | 73.973 | 1.00 | 59.97 | N |
| ATOM | 7129 | CE1 | HIS | C | 155 | −32.055 | −15.402 | 74.952 | 1.00 | 64.00 | C |
| ATOM | 7130 | NE2 | HIS | C | 155 | −33.103 | −14.617 | 75.099 | 1.00 | 67.01 | N |
| ATOM | 7131 | CD2 | HIS | C | 155 | −34.063 | −14.974 | 74.182 | 1.00 | 63.85 | C |
| ATOM | 7132 | C | HIS | C | 155 | −36.100 | −18.169 | 71.599 | 1.00 | 49.24 | C |
| ATOM | 7133 | O | HIS | C | 155 | −37.151 | −17.632 | 71.362 | 1.00 | 47.14 | O |
| ATOM | 7134 | N | ALA | C | 156 | −35.588 | −19.105 | 70.823 | 1.00 | 50.90 | N |
| ATOM | 7135 | CA | ALA | C | 156 | −36.284 | −19.652 | 69.651 | 1.00 | 52.20 | C |
| ATOM | 7136 | CB | ALA | C | 156 | −35.401 | −20.668 | 68.933 | 1.00 | 59.25 | C |
| ATOM | 7137 | C | ALA | C | 156 | −37.641 | −20.263 | 69.974 | 1.00 | 49.25 | C |
| ATOM | 7138 | O | ALA | C | 156 | −38.626 | −19.977 | 69.333 | 1.00 | 45.92 | O |
| ATOM | 7139 | N | ASN | C | 157 | −37.683 | −21.049 | 71.040 | 1.00 | 54.03 | N |
| ATOM | 7140 | CA | ASN | C | 157 | −38.916 | −21.609 | 71.572 | 1.00 | 44.29 | C |
| ATOM | 7141 | CB | ASN | C | 157 | −38.614 | −22.562 | 72.720 | 1.00 | 41.46 | C |
| ATOM | 7142 | CG | ASN | C | 157 | −38.132 | −23.903 | 72.239 | 1.00 | 47.75 | C |
| ATOM | 7143 | OD1 | ASN | C | 157 | −38.932 | −24.808 | 72.007 | 1.00 | 50.40 | O |
| ATOM | 7144 | ND2 | ASN | C | 157 | −36.820 | −24.042 | 72.073 | 1.00 | 50.90 | N |
| ATOM | 7145 | C | ASN | C | 157 | −39.899 | −20.532 | 72.034 | 1.00 | 42.62 | C |
| ATOM | 7146 | O | ASN | C | 157 | −41.073 | −20.606 | 71.766 | 1.00 | 49.55 | O |
| ATOM | 7147 | N | LEU | C | 158 | −39.407 | −19.523 | 72.734 | 1.00 | 43.23 | N |
| ATOM | 7148 | CA | LEU | C | 158 | −40.228 | −18.399 | 73.160 | 1.00 | 44.22 | C |
| ATOM | 7149 | CB | LEU | C | 158 | −39.401 | −17.438 | 74.012 | 1.00 | 46.76 | C |
| ATOM | 7150 | CG | LEU | C | 158 | −40.040 | −16.086 | 74.319 | 1.00 | 45.53 | C |
| ATOM | 7151 | CD1 | LEU | C | 158 | −41.263 | −16.269 | 75.193 | 1.00 | 48.33 | C |
| ATOM | 7152 | CD2 | LEU | C | 158 | −39.032 | −15.171 | 74.984 | 1.00 | 52.55 | C |
| ATOM | 7153 | C | LEU | C | 158 | −40.853 | −17.645 | 71.981 | 1.00 | 46.40 | C |
| ATOM | 7154 | O | LEU | C | 158 | −42.037 | −17.412 | 71.930 | 1.00 | 43.73 | O |
| ATOM | 7155 | N | ILE | C | 159 | −40.056 | −17.370 | 70.978 | 1.00 | 44.76 | N |
| ATOM | 7156 | CA | ILE | C | 159 | −40.535 | −16.785 | 69.751 | 1.00 | 41.12 | C |
| ATOM | 7157 | CB | ILE | C | 159 | −39.379 | −16.465 | 68.800 | 1.00 | 42.84 | C |
| ATOM | 7158 | CG1 | ILE | C | 159 | −38.488 | −15.396 | 69.431 | 1.00 | 38.15 | C |
| ATOM | 7159 | CD1 | ILE | C | 159 | −37.382 | −14.921 | 68.532 | 1.00 | 50.51 | C |
| ATOM | 7160 | CG2 | ILE | C | 159 | −39.906 | −16.000 | 67.453 | 1.00 | 44.13 | C |
| ATOM | 7161 | C | ILE | C | 159 | −41.571 | −17.676 | 69.064 | 1.00 | 43.26 | C |
| ATOM | 7162 | O | ILE | C | 159 | −42.597 | −17.223 | 68.618 | 1.00 | 46.98 | O |
| ATOM | 7163 | N | ALA | C | 160 | −41.321 | −18.966 | 69.038 | 1.00 | 40.15 | N |
| ATOM | 7164 | CA | ALA | C | 160 | −42.234 | −19.912 | 68.435 | 1.00 | 39.06 | C |
| ATOM | 7165 | CB | ALA | C | 160 | −41.613 | −21.300 | 68.401 | 1.00 | 40.24 | C |
| ATOM | 7166 | C | ALA | C | 160 | −43.610 | −19.961 | 69.117 | 1.00 | 39.81 | C |
| ATOM | 7167 | O | ALA | C | 160 | −44.632 | −20.065 | 68.473 | 1.00 | 39.04 | O |
| ATOM | 7168 | N | ALA | C | 161 | −43.635 | −19.855 | 70.428 | 1.00 | 33.71 | N |
| ATOM | 7169 | CA | ALA | C | 161 | −44.885 | −19.824 | 71.171 | 1.00 | 42.47 | C |
| ATOM | 7170 | CB | ALA | C | 161 | −44.599 | −19.851 | 72.664 | 1.00 | 43.56 | C |
| ATOM | 7171 | C | ALA | C | 161 | −45.770 | −18.628 | 70.816 | 1.00 | 45.69 | C |
| ATOM | 7172 | O | ALA | C | 161 | −46.962 | −18.742 | 70.612 | 1.00 | 48.41 | O |
| ATOM | 7173 | N | PHE | C | 162 | −45.138 | −17.474 | 70.731 | 1.00 | 42.24 | N |
| ATOM | 7174 | CA | PHE | C | 162 | −45.747 | −16.239 | 70.295 | 1.00 | 42.01 | C |
| ATOM | 7175 | CB | PHE | C | 162 | −44.856 | −15.034 | 70.590 | 1.00 | 37.60 | C |
| ATOM | 7176 | CG | PHE | C | 162 | −44.971 | −14.558 | 72.009 | 1.00 | 35.48 | C |
| ATOM | 7177 | CD2 | PHE | C | 162 | −45.825 | −13.526 | 72.335 | 1.00 | 40.02 | C |
| ATOM | 7178 | CE2 | PHE | C | 162 | −45.950 | −13.098 | 73.644 | 1.00 | 47.26 | C |
| ATOM | 7179 | CZ | PHE | C | 162 | −45.230 | −13.710 | 74.646 | 1.00 | 39.20 | C |
| ATOM | 7180 | CE1 | PHE | C | 162 | −44.386 | −14.746 | 74.339 | 1.00 | 43.42 | C |
| ATOM | 7181 | CD1 | PHE | C | 162 | −44.262 | −15.173 | 73.025 | 1.00 | 43.72 | C |
| ATOM | 7182 | C | PHE | C | 162 | −46.248 | −16.321 | 68.859 | 1.00 | 45.24 | C |
| ATOM | 7183 | O | PHE | C | 162 | −47.305 | −15.837 | 68.533 | 1.00 | 46.74 | O |
| ATOM | 7184 | N | ILE | C | 163 | −45.476 | −16.939 | 67.980 | 1.00 | 43.73 | N |
| ATOM | 7185 | CA | ILE | C | 163 | −45.902 | −17.092 | 66.593 | 1.00 | 43.35 | C |
| ATOM | 7186 | CB | ILE | C | 163 | −44.842 | −17.835 | 65.753 | 1.00 | 40.00 | C |
| ATOM | 7187 | CG1 | ILE | C | 163 | −43.555 | −17.021 | 65.664 | 1.00 | 38.39 | C |
| ATOM | 7188 | CD1 | ILE | C | 163 | −42.425 | −17.752 | 64.975 | 1.00 | 41.68 | C |
| ATOM | 7189 | CG2 | ILE | C | 163 | −45.371 | −18.136 | 64.354 | 1.00 | 36.66 | C |
| ATOM | 7190 | C | ILE | C | 163 | −47.181 | −17.890 | 66.505 | 1.00 | 42.91 | C |
| ATOM | 7191 | O | ILE | C | 163 | −48.117 | −17.522 | 65.831 | 1.00 | 43.72 | O |
| ATOM | 7192 | N | LEU | C | 164 | −47.210 | −18.993 | 67.225 | 1.00 | 46.17 | N |
| ATOM | 7193 | CA | LEU | C | 164 | −48.356 | −19.887 | 67.223 | 1.00 | 45.15 | C |
| ATOM | 7194 | CB | LEU | C | 164 | −48.065 | −21.161 | 68.021 | 1.00 | 45.34 | C |
| ATOM | 7195 | CG | LEU | C | 164 | −47.107 | −22.157 | 67.362 | 1.00 | 47.05 | C |
| ATOM | 7196 | CD1 | LEU | C | 164 | −46.954 | −23.399 | 68.222 | 1.00 | 45.29 | C |
| ATOM | 7197 | CD2 | LEU | C | 164 | −47.587 | −22.529 | 65.966 | 1.00 | 46.65 | C |
| ATOM | 7198 | C | LEU | C | 164 | −49.598 | −19.213 | 67.743 | 1.00 | 46.69 | C |
| ATOM | 7199 | O | LEU | C | 164 | −50.648 | −19.360 | 67.167 | 1.00 | 52.09 | O |
| ATOM | 7200 | N | ARG | C | 165 | −49.473 | −18.436 | 68.803 | 1.00 | 44.14 | N |
| ATOM | 7201 | CA | ARG | C | 165 | −50.584 | −17.624 | 69.248 | 1.00 | 46.97 | C |
| ATOM | 7202 | CB | ARG | C | 165 | −50.197 | −16.860 | 70.517 | 1.00 | 46.93 | C |
| ATOM | 7203 | CG | ARG | C | 165 | −51.228 | −15.839 | 70.970 | 1.00 | 53.08 | C |
| ATOM | 7204 | CD | ARG | C | 165 | −50.959 | −15.393 | 72.399 | 1.00 | 53.29 | C |
| ATOM | 7205 | NE | ARG | C | 165 | −52.070 | −14.632 | 72.967 | 1.00 | 67.02 | N |
| ATOM | 7206 | CZ | ARG | C | 165 | −53.183 | −15.180 | 73.450 | 1.00 | 66.76 | C |
| ATOM | 7207 | NH1 | ARG | C | 165 | −53.346 | −16.498 | 73.425 | 1.00 | 63.35 | N |
| ATOM | 7208 | NH2 | ARG | C | 165 | −54.141 | −14.412 | 73.954 | 1.00 | 64.88 | N |
| ATOM | 7209 | C | ARG | C | 165 | −51.091 | −16.632 | 68.190 | 1.00 | 44.70 | C |
| ATOM | 7210 | O | ARG | C | 165 | −52.264 | −16.532 | 67.930 | 1.00 | 50.98 | O |
| ATOM | 7211 | N | ASN | C | 166 | −50.200 | −15.891 | 67.580 | 1.00 | 43.44 | N |
| ATOM | 7212 | CA | ASN | C | 166 | −50.581 | −14.916 | 66.561 | 1.00 | 45.88 | C |
| ATOM | 7213 | CB | ASN | C | 166 | −49.408 | −14.023 | 66.168 | 1.00 | 52.92 | C |
| ATOM | 7214 | CG | ASN | C | 166 | −49.004 | −13.088 | 67.289 | 1.00 | 59.91 | C |
| ATOM | 7215 | OD1 | ASN | C | 166 | −49.428 | −13.258 | 68.434 | 1.00 | 61.73 | O |
| ATOM | 7216 | ND2 | ASN | C | 166 | −48.186 | −12.094 | 66.968 | 1.00 | 63.37 | N |
| ATOM | 7217 | C | ASN | C | 166 | −51.272 | −15.540 | 65.358 | 1.00 | 46.22 | C |
| ATOM | 7218 | O | ASN | C | 166 | −52.284 | −15.077 | 64.876 | 1.00 | 52.33 | O |
| ATOM | 7219 | N | ALA | C | 167 | −50.756 | −16.653 | 64.916 | 1.00 | 44.19 | N |
| ATOM | 7220 | CA | ALA | C | 167 | −51.391 | −17.418 | 63.870 | 1.00 | 47.25 | C |
| ATOM | 7221 | CB | ALA | C | 167 | −50.520 | −18.598 | 63.474 | 1.00 | 42.93 | C |
| ATOM | 7222 | C | ALA | C | 167 | −52.811 | −17.909 | 64.277 | 1.00 | 49.85 | C |
| ATOM | 7223 | O | ALA | C | 167 | −53.787 | −17.767 | 63.561 | 1.00 | 48.10 | O |
| ATOM | 7224 | N | THR | C | 168 | −52.918 | −18.433 | 65.487 | 1.00 | 47.50 | N |
| ATOM | 7225 | CA | THR | C | 168 | −54.169 | −18.909 | 66.037 | 1.00 | 46.73 | C |
| ATOM | 7226 | CB | THR | C | 168 | −53.967 | −19.546 | 67.431 | 1.00 | 48.57 | C |
| ATOM | 7227 | OG1 | THR | C | 168 | −52.977 | −20.576 | 67.341 | 1.00 | 43.50 | O |
| ATOM | 7228 | CG2 | THR | C | 168 | −55.264 | −20.152 | 67.951 | 1.00 | 46.90 | C |
| ATOM | 7229 | C | THR | C | 168 | −55.192 | −17.805 | 66.117 | 1.00 | 46.17 | C |
| ATOM | 7230 | O | THR | C | 168 | −56.357 | −18.042 | 65.915 | 1.00 | 47.35 | O |
| ATOM | 7231 | N | TRP | C | 169 | −54.751 | −16.583 | 66.408 | 1.00 | 44.23 | N |
| ATOM | 7232 | CA | TRP | C | 169 | −55.670 | −15.449 | 66.500 | 1.00 | 44.82 | C |
| ATOM | 7233 | CB | TRP | C | 169 | −54.901 | −14.178 | 66.847 | 1.00 | 39.60 | C |
| ATOM | 7234 | CG | TRP | C | 169 | −55.755 | −12.958 | 67.012 | 1.00 | 43.26 | C |
| ATOM | 7235 | CD1 | TRP | C | 169 | −56.248 | −12.464 | 68.183 | 1.00 | 46.00 | C |
| ATOM | 7236 | NE1 | TRP | C | 169 | −56.971 | −11.324 | 67.951 | 1.00 | 46.60 | N |
| ATOM | 7237 | CE2 | TRP | C | 169 | −56.956 | −11.055 | 66.609 | 1.00 | 39.60 | C |
| ATOM | 7238 | CD2 | TRP | C | 169 | −56.197 | −12.061 | 65.982 | 1.00 | 39.55 | C |
| ATOM | 7239 | CE3 | TRP | C | 169 | −56.028 | −12.012 | 64.596 | 1.00 | 44.10 | C |
| ATOM | 7240 | CZ3 | TRP | C | 169 | −56.618 | −10.972 | 63.895 | 1.00 | 46.71 | C |
| ATOM | 7241 | CH2 | TRP | C | 169 | −57.365 | −9.990 | 64.551 | 1.00 | 42.91 | C |
| ATOM | 7242 | CZ2 | TRP | C | 169 | −57.544 | −10.015 | 65.902 | 1.00 | 41.08 | C |
| ATOM | 7243 | C | TRP | C | 169 | −56.446 | −15.238 | 65.197 | 1.00 | 48.90 | C |
| ATOM | 7244 | O | TRP | C | 169 | −57.633 | −14.981 | 65.208 | 1.00 | 48.83 | O |
| ATOM | 7245 | N | PHE | C | 170 | −55.769 | −15.370 | 64.064 | 1.00 | 48.94 | N |
| ATOM | 7246 | CA | PHE | C | 170 | −56.453 | −15.319 | 62.777 | 1.00 | 43.37 | C |
| ATOM | 7247 | CB | PHE | C | 170 | −55.476 | −15.369 | 61.605 | 1.00 | 38.64 | C |
| ATOM | 7248 | CG | PHE | C | 170 | −54.664 | −14.119 | 61.464 | 1.00 | 43.67 | C |
| ATOM | 7249 | CD1 | PHE | C | 170 | −55.240 | −12.958 | 60.980 | 1.00 | 38.11 | C |
| ATOM | 7250 | CE1 | PHE | C | 170 | −54.498 | −11.801 | 60.859 | 1.00 | 54.58 | C |
| ATOM | 7251 | CZ | PHE | C | 170 | −53.164 | −11.791 | 61.230 | 1.00 | 53.28 | C |
| ATOM | 7252 | CE2 | PHE | C | 170 | −52.580 | −12.939 | 61.720 | 1.00 | 47.02 | C |
| ATOM | 7253 | CD2 | PHE | C | 170 | −53.329 | −14.095 | 61.838 | 1.00 | 50.60 | C |
| ATOM | 7254 | C | PHE | C | 170 | −57.535 | −16.370 | 62.681 | 1.00 | 46.08 | C |
| ATOM | 7255 | O | PHE | C | 170 | −58.646 | −16.095 | 62.284 | 1.00 | 53.84 | O |
| ATOM | 7256 | N | VAL | C | 171 | −57.213 | −17.581 | 63.093 | 1.00 | 49.49 | N |
| ATOM | 7257 | CA | VAL | C | 171 | −58.199 | −18.636 | 63.109 | 1.00 | 50.18 | C |
| ATOM | 7258 | CB | VAL | C | 171 | −57.569 | −19.971 | 63.577 | 1.00 | 46.73 | C |
| ATOM | 7259 | CG1 | VAL | C | 171 | −58.593 | −21.097 | 63.543 | 1.00 | 34.24 | C |
| ATOM | 7260 | CG2 | VAL | C | 171 | −56.353 | −20.316 | 62.728 | 1.00 | 41.02 | C |
| ATOM | 7261 | C | VAL | C | 171 | −59.368 | −18.308 | 64.029 | 1.00 | 52.08 | C |
| ATOM | 7262 | O | VAL | C | 171 | −60.507 | −18.494 | 63.664 | 1.00 | 60.75 | O |
| ATOM | 7263 | N | VAL | C | 172 | −59.085 | −17.858 | 65.244 | 1.00 | 48.70 | N |
| ATOM | 7264 | CA | VAL | C | 172 | −60.138 | −17.542 | 66.195 | 1.00 | 50.07 | C |
| ATOM | 7265 | CB | VAL | C | 172 | −59.521 | −17.026 | 67.524 | 1.00 | 45.71 | C |
| ATOM | 7266 | CG1 | VAL | C | 172 | −60.586 | −16.466 | 68.461 | 1.00 | 48.11 | C |
| ATOM | 7267 | CG2 | VAL | C | 172 | −58.737 | −18.131 | 68.207 | 1.00 | 49.77 | C |
| ATOM | 7268 | C | VAL | C | 172 | −61.144 | −16.512 | 65.690 | 1.00 | 55.61 | C |
| ATOM | 7269 | O | VAL | C | 172 | −62.321 | −16.583 | 65.985 | 1.00 | 55.85 | O |
| ATOM | 7270 | N | GLN | C | 173 | −60.679 | −15.616 | 64.830 | 1.00 | 58.41 | N |
| ATOM | 7271 | CA | GLN | C | 173 | −61.539 | −14.648 | 64.179 | 1.00 | 61.35 | C |
| ATOM | 7272 | CB | GLN | C | 173 | −60.708 | −13.594 | 63.444 | 1.00 | 57.11 | C |
| ATOM | 7273 | CG | GLN | C | 173 | −59.868 | −12.734 | 64.383 | 1.00 | 49.17 | C |
| ATOM | 7274 | CD | GLN | C | 173 | −60.709 | −12.017 | 65.428 | 1.00 | 53.48 | C |
| ATOM | 7275 | OE1 | GLN | C | 173 | −61.595 | −11.232 | 65.094 | 1.00 | 63.85 | O |
| ATOM | 7276 | NE2 | GLN | C | 173 | −60.438 | −12.290 | 66.700 | 1.00 | 46.26 | N |
| ATOM | 7277 | C | GLN | C | 173 | −62.636 | −15.229 | 63.269 | 1.00 | 68.11 | C |
| ATOM | 7278 | O | GLN | C | 173 | −63.760 | −14.754 | 63.243 | 1.00 | 76.36 | O |
| ATOM | 7279 | N | LEU | C | 174 | −62.346 | −16.343 | 62.610 | 1.00 | 67.70 | N |
| ATOM | 7280 | CA | LEU | C | 174 | −63.369 | −17.071 | 61.870 | 1.00 | 63.49 | C |
| ATOM | 7281 | CB | LEU | C | 174 | −62.729 | −18.168 | 61.017 | 1.00 | 64.45 | C |
| ATOM | 7282 | CG | LEU | C | 174 | −61.464 | −17.710 | 60.271 | 1.00 | 72.83 | C |
| ATOM | 7283 | CD1 | LEU | C | 174 | −60.904 | −18.813 | 59.376 | 1.00 | 72.53 | C |
| ATOM | 7284 | CD2 | LEU | C | 174 | −61.708 | −16.432 | 59.466 | 1.00 | 70.23 | C |
| ATOM | 7285 | C | LEU | C | 174 | −64.470 | −17.634 | 62.802 | 1.00 | 69.48 | C |
| ATOM | 7286 | O | LEU | C | 174 | −65.657 | −17.587 | 62.523 | 1.00 | 78.42 | O |
| ATOM | 7287 | N | THR | C | 175 | −64.030 | −18.073 | 63.983 | 1.00 | 70.94 | N |
| ATOM | 7288 | CA | THR | C | 175 | −64.895 | −18.589 | 65.053 | 1.00 | 78.39 | C |
| ATOM | 7289 | CB | THR | C | 175 | −64.119 | −19.442 | 66.063 | 1.00 | 83.59 | C |
| ATOM | 7290 | OG1 | THR | C | 175 | −63.265 | −18.596 | 66.844 | 1.00 | 82.70 | O |
| ATOM | 7291 | CG2 | THR | C | 175 | −63.284 | −20.494 | 65.341 | 1.00 | 81.41 | C |
| ATOM | 7292 | C | THR | C | 175 | −65.625 | −17.462 | 65.813 | 1.00 | 80.02 | C |
| ATOM | 7293 | O | THR | C | 175 | −66.422 | −17.700 | 66.720 | 1.00 | 76.79 | O |
| ATOM | 7294 | N | MET | C | 176 | −65.310 | −16.218 | 65.462 | 1.00 | 82.01 | N |
| ATOM | 7295 | CA | MET | C | 176 | −65.924 | −15.074 | 66.114 | 1.00 | 85.76 | C |
| ATOM | 7296 | CB | MET | C | 176 | −65.192 | −13.767 | 65.787 | 1.00 | 83.75 | C |
| ATOM | 7297 | CG | MET | C | 176 | −65.396 | −12.673 | 66.830 | 1.00 | 89.26 | C |
| ATOM | 7298 | SD | MET | C | 176 | −65.060 | −13.233 | 68.517 | 1.00 | 108.15 | S |
| ATOM | 7299 | CE | MET | C | 176 | −63.273 | −13.382 | 68.481 | 1.00 | 70.88 | C |
| ATOM | 7300 | C | MET | C | 176 | −67.437 | −14.937 | 65.873 | 1.00 | 89.84 | C |
| ATOM | 7301 | O | MET | C | 176 | −68.107 | −14.219 | 66.616 | 1.00 | 88.33 | O |
| ATOM | 7302 | O | SER | C | 177 | −70.269 | −16.648 | 65.881 | 1.00 | 89.89 | O |
| ATOM | 7303 | N | SER | C | 177 | −67.941 | −15.467 | 64.729 | 1.00 | 90.72 | N |
| ATOM | 7304 | CA | SER | C | 177 | −69.306 | −15.102 | 64.268 | 1.00 | 87.48 | C |
| ATOM | 7305 | C | SER | C | 177 | −70.440 | −15.642 | 65.203 | 1.00 | 91.83 | C |
| ATOM | 7306 | CB | SER | C | 177 | −69.530 | −15.605 | 62.842 | 1.00 | 78.47 | C |
| ATOM | 7307 | OG | SER | C | 177 | −68.463 | −15.220 | 61.996 | 1.00 | 76.56 | O |
| ATOM | 7308 | O | PRO | C | 178 | −73.556 | −17.288 | 67.191 | 1.00 | 92.77 | O |
| ATOM | 7309 | N | PRO | C | 178 | −71.593 | −14.907 | 65.390 | 1.00 | 93.66 | N |
| ATOM | 7310 | CA | PRO | C | 178 | −72.629 | −15.204 | 66.416 | 1.00 | 94.11 | C |
| ATOM | 7311 | C | PRO | C | 178 | −73.298 | −16.576 | 66.214 | 1.00 | 96.69 | C |
| ATOM | 7312 | CB | PRO | C | 178 | −73.644 | −14.075 | 66.221 | 1.00 | 94.36 | C |
| ATOM | 7313 | CG | PRO | C | 178 | −72.854 | −12.964 | 65.603 | 1.00 | 82.98 | C |
| ATOM | 7314 | CD | PRO | C | 178 | −71.877 | −13.645 | 64.690 | 1.00 | 89.52 | C |
| ATOM | 7315 | O | GLU | C | 179 | −73.606 | −20.255 | 65.938 | 1.00 | 92.30 | O |
| ATOM | 7316 | N | GLU | C | 179 | −73.522 | −16.946 | 64.940 | 1.00 | 94.55 | N |
| ATOM | 7317 | CA | GLU | C | 179 | −73.987 | −18.292 | 64.595 | 1.00 | 97.18 | C |
| ATOM | 7318 | C | GLU | C | 179 | −73.109 | −19.353 | 65.271 | 1.00 | 95.02 | C |
| ATOM | 7319 | CB | GLU | C | 179 | −73.921 | −18.519 | 63.083 | 1.00 | 107.90 | C |
| ATOM | 7320 | CG | GLU | C | 179 | −74.867 | −17.682 | 62.243 | 1.00 | 115.30 | C |
| ATOM | 7321 | CD | GLU | C | 179 | −74.779 | −18.037 | 60.769 | 1.00 | 118.54 | C |
| ATOM | 7322 | OE1 | GLU | C | 179 | −73.991 | −18.944 | 60.422 | 1.00 | 120.90 | O |
| ATOM | 7323 | OE2 | GLU | C | 179 | −75.494 | −17.411 | 59.959 | 1.00 | 119.51 | O |
| ATOM | 7324 | N | VAL | C | 180 | −71.779 | −19.203 | 65.046 | 1.00 | 99.52 | N |
| ATOM | 7325 | CA | VAL | C | 180 | −70.756 | −20.124 | 65.545 | 1.00 | 90.38 | C |
| ATOM | 7326 | CB | VAL | C | 180 | −69.356 | −19.734 | 65.036 | 1.00 | 82.98 | C |
| ATOM | 7327 | CG1 | VAL | C | 180 | −68.339 | −20.793 | 65.426 | 1.00 | 84.63 | C |
| ATOM | 7328 | CG2 | VAL | C | 180 | −69.376 | −19.539 | 63.525 | 1.00 | 82.01 | C |
| ATOM | 7329 | C | VAL | C | 180 | −70.732 | −20.196 | 67.069 | 1.00 | 87.86 | C |
| ATOM | 7330 | O | VAL | C | 180 | −70.780 | −21.268 | 67.621 | 1.00 | 83.56 | O |
| ATOM | 7331 | N | HIS | C | 181 | −70.747 | −19.056 | 67.752 | 1.00 | 93.77 | N |
| ATOM | 7332 | CA | HIS | C | 181 | −70.860 | −19.063 | 69.225 | 1.00 | 99.26 | C |
| ATOM | 7333 | CB | HIS | C | 181 | −70.887 | −17.627 | 69.764 | 1.00 | 102.37 | C |
| ATOM | 7334 | CG | HIS | C | 181 | −69.539 | −17.080 | 70.120 | 1.00 | 108.41 | C |
| ATOM | 7335 | ND1 | HIS | C | 181 | −68.819 | −17.525 | 71.207 | 1.00 | 104.53 | N |
| ATOM | 7336 | CE1 | HIS | C | 181 | −67.678 | −16.860 | 71.278 | 1.00 | 109.29 | C |
| ATOM | 7337 | NE2 | HIS | C | 181 | −67.636 | −15.996 | 70.282 | 1.00 | 121.56 | N |
| ATOM | 7338 | CD2 | HIS | C | 181 | −68.792 | −16.109 | 69.544 | 1.00 | 117.06 | C |
| ATOM | 7339 | C | HIS | C | 181 | −72.127 | −19.821 | 69.763 | 1.00 | 99.72 | C |
| ATOM | 7340 | O | HIS | C | 181 | −72.045 | −20.611 | 70.711 | 1.00 | 89.86 | O |
| ATOM | 7341 | N | GLN | C | 182 | −73.307 | −19.545 | 69.160 | 1.00 | 96.98 | N |
| ATOM | 7342 | CA | GLN | C | 182 | −74.558 | −20.183 | 69.604 | 1.00 | 93.28 | C |
| ATOM | 7343 | CB | GLN | C | 182 | −75.780 | −19.515 | 68.968 | 1.00 | 96.23 | C |
| ATOM | 7344 | CG | GLN | C | 182 | −76.034 | −18.117 | 69.528 | 1.00 | 103.95 | C |
| ATOM | 7345 | CD | GLN | C | 182 | −77.261 | −17.448 | 68.941 | 1.00 | 104.53 | C |
| ATOM | 7346 | OE1 | GLN | C | 182 | −77.745 | −17.841 | 67.881 | 1.00 | 106.81 | O |
| ATOM | 7347 | NE2 | GLN | C | 182 | −77.771 | −16.429 | 69.631 | 1.00 | 94.37 | N |
| ATOM | 7348 | C | GLN | C | 182 | −74.580 | −21.696 | 69.426 | 1.00 | 92.34 | C |
| ATOM | 7349 | O | GLN | C | 182 | −75.229 | −22.394 | 70.196 | 1.00 | 98.48 | O |
| ATOM | 7350 | N | SER | C | 183 | −73.754 | −22.198 | 68.493 | 1.00 | 89.54 | N |
| ATOM | 7351 | CA | SER | C | 183 | −73.599 | −23.639 | 68.340 | 1.00 | 89.43 | C |
| ATOM | 7352 | CB | SER | C | 183 | −73.624 | −24.031 | 66.862 | 1.00 | 94.35 | C |
| ATOM | 7353 | OG | SER | C | 183 | −72.546 | −23.440 | 66.154 | 1.00 | 90.28 | O |
| ATOM | 7354 | C | SER | C | 183 | −72.270 | −24.031 | 68.960 | 1.00 | 83.62 | C |
| ATOM | 7355 | O | SER | C | 183 | −71.250 | −23.476 | 68.633 | 1.00 | 83.94 | O |
| ATOM | 7356 | N | ASN | C | 184 | −72.291 | −24.916 | 69.942 | 1.00 | 82.31 | N |
| ATOM | 7357 | CA | ASN | C | 184 | −71.068 | −25.187 | 70.673 | 1.00 | 81.45 | C |
| ATOM | 7358 | CB | ASN | C | 184 | −71.439 | −25.521 | 72.119 | 1.00 | 78.64 | C |
| ATOM | 7359 | CG | ASN | C | 184 | −70.381 | −25.105 | 73.111 | 1.00 | 73.94 | C |
| ATOM | 7360 | OD1 | ASN | C | 184 | −69.829 | −24.013 | 73.019 | 1.00 | 79.18 | O |
| ATOM | 7361 | ND2 | ASN | C | 184 | −70.101 | −25.969 | 74.079 | 1.00 | 71.16 | N |
| ATOM | 7362 | C | ASN | C | 184 | −70.385 | −26.395 | 70.063 | 1.00 | 80.36 | C |
| ATOM | 7363 | O | ASN | C | 184 | −70.261 | −27.451 | 70.675 | 1.00 | 81.38 | O |
| ATOM | 7364 | N | VAL | C | 185 | −70.006 | −26.253 | 68.795 | 1.00 | 77.60 | N |
| ATOM | 7365 | CA | VAL | C | 185 | −69.536 | −27.394 | 68.078 | 1.00 | 75.21 | C |
| ATOM | 7366 | CB | VAL | C | 185 | −69.494 | −27.087 | 66.556 | 1.00 | 78.36 | C |
| ATOM | 7367 | CG1 | VAL | C | 185 | −69.243 | −28.348 | 65.737 | 1.00 | 79.83 | C |
| ATOM | 7368 | CG2 | VAL | C | 185 | −70.798 | −26.426 | 66.113 | 1.00 | 96.35 | C |
| ATOM | 7369 | C | VAL | C | 185 | −68.159 | −27.746 | 68.568 | 1.00 | 74.34 | C |
| ATOM | 7370 | O | VAL | C | 185 | −67.493 | −26.935 | 69.179 | 1.00 | 76.15 | O |
| ATOM | 7371 | O | GLY | C | 186 | −64.335 | −28.665 | 69.009 | 1.00 | 54.97 | O |
| ATOM | 7372 | N | GLY | C | 186 | −67.748 | −28.968 | 68.287 | 1.00 | 75.89 | N |
| ATOM | 7373 | CA | GLY | C | 186 | −66.491 | −29.499 | 68.690 | 1.00 | 66.80 | C |
| ATOM | 7374 | C | GLY | C | 186 | −65.290 | −28.743 | 68.261 | 1.00 | 61.21 | C |
| ATOM | 7375 | O | TRP | C | 187 | −62.917 | −25.663 | 67.154 | 1.00 | 58.15 | O |
| ATOM | 7376 | N | TRP | C | 187 | −65.261 | −28.280 | 67.018 | 1.00 | 62.90 | N |
| ATOM | 7377 | CA | TRP | C | 187 | −64.053 | −27.665 | 66.560 | 1.00 | 66.39 | C |
| ATOM | 7378 | C | TRP | C | 187 | −63.986 | −26.211 | 66.987 | 1.00 | 62.06 | C |
| ATOM | 7379 | CB | TRP | C | 187 | −63.988 | −27.753 | 65.025 | 1.00 | 68.78 | C |
| ATOM | 7380 | CG | TRP | C | 187 | −64.978 | −26.860 | 64.346 | 1.00 | 75.01 | C |
| ATOM | 7381 | CD1 | TRP | C | 187 | −66.268 | −27.162 | 64.033 | 1.00 | 77.90 | C |
| ATOM | 7382 | CD2 | TRP | C | 187 | −64.765 | −25.506 | 63.918 | 1.00 | 79.27 | C |
| ATOM | 7383 | NE1 | TRP | C | 187 | −66.876 | −26.082 | 63.439 | 1.00 | 79.49 | N |
| ATOM | 7384 | CE2 | TRP | C | 187 | −65.977 | −25.052 | 63.357 | 1.00 | 77.61 | C |
| ATOM | 7385 | CE3 | TRP | C | 187 | −63.672 | −24.632 | 63.958 | 1.00 | 72.78 | C |
| ATOM | 7386 | CZ2 | TRP | C | 187 | −66.127 | −23.769 | 62.837 | 1.00 | 78.36 | C |
| ATOM | 7387 | CZ3 | TRP | C | 187 | −63.823 | −23.352 | 63.438 | 1.00 | 73.51 | C |
| ATOM | 7388 | CH2 | TRP | C | 187 | −65.043 | −22.934 | 62.887 | 1.00 | 88.41 | C |
| ATOM | 7389 | O | CYS | C | 188 | −63.636 | −23.434 | 69.542 | 1.00 | 57.29 | O |
| ATOM | 7390 | N | CYS | C | 188 | −65.134 | −25.637 | 67.320 | 1.00 | 72.39 | N |
| ATOM | 7391 | CA | CYS | C | 188 | −65.165 | −24.335 | 67.943 | 1.00 | 67.22 | C |
| ATOM | 7392 | C | CYS | C | 188 | −64.388 | −24.335 | 69.246 | 1.00 | 63.23 | C |
| ATOM | 7393 | CB | CYS | C | 188 | −66.606 | −23.894 | 68.190 | 1.00 | 70.55 | C |
| ATOM | 7394 | SG | CYS | C | 188 | −66.760 | −22.131 | 68.506 | 1.00 | 93.84 | S |
| ATOM | 7395 | N | ARG | C | 189 | −64.616 | −25.368 | 70.033 | 1.00 | 62.63 | N |
| ATOM | 7396 | CA | ARG | C | 189 | −63.991 | −25.490 | 71.317 | 1.00 | 56.36 | C |
| ATOM | 7397 | CB | ARG | C | 189 | −64.751 | −26.504 | 72.173 | 1.00 | 60.13 | C |
| ATOM | 7398 | CG | ARG | C | 189 | −66.210 | −26.152 | 72.424 | 1.00 | 64.44 | C |
| ATOM | 7399 | CD | ARG | C | 189 | −66.931 | −27.287 | 73.149 | 1.00 | 74.77 | C |
| ATOM | 7400 | NE | ARG | C | 189 | −67.791 | −28.054 | 72.249 | 1.00 | 74.18 | N |
| ATOM | 7401 | CZ | ARG | C | 189 | −68.295 | −29.251 | 72.532 | 1.00 | 71.65 | C |
| ATOM | 7402 | NH1 | ARG | C | 189 | −68.018 | −29.840 | 73.691 | 1.00 | 69.37 | N |
| ATOM | 7403 | NH2 | ARG | C | 189 | −69.074 | −29.864 | 71.649 | 1.00 | 74.55 | N |
| ATOM | 7404 | C | ARG | C | 189 | −62.539 | −25.912 | 71.179 | 1.00 | 53.51 | C |
| ATOM | 7405 | O | ARG | C | 189 | −61.716 | −25.567 | 71.991 | 1.00 | 55.61 | O |
| ATOM | 7406 | N | LEU | C | 190 | −62.201 | −26.630 | 70.118 | 1.00 | 54.10 | N |
| ATOM | 7407 | CA | LEU | C | 190 | −60.816 | −26.987 | 69.902 | 1.00 | 48.19 | C |
| ATOM | 7408 | CB | LEU | C | 190 | −60.667 | −27.916 | 68.694 | 1.00 | 44.07 | C |
| ATOM | 7409 | CG | LEU | C | 190 | −59.251 | −28.396 | 68.371 | 1.00 | 31.81 | C |
| ATOM | 7410 | CD1 | LEU | C | 190 | −58.662 | −29.212 | 69.513 | 1.00 | 34.94 | C |
| ATOM | 7411 | CD2 | LEU | C | 190 | −59.249 | −29.200 | 67.087 | 1.00 | 30.84 | C |
| ATOM | 7412 | C | LEU | C | 190 | −59.964 | −25.758 | 69.733 | 1.00 | 48.47 | C |
| ATOM | 7413 | O | LEU | C | 190 | −58.904 | −25.656 | 70.299 | 1.00 | 47.95 | O |
| ATOM | 7414 | N | VAL | C | 191 | −60.430 | −24.821 | 68.923 | 1.00 | 48.65 | N |
| ATOM | 7415 | CA | VAL | C | 191 | −59.669 | −23.624 | 68.680 | 1.00 | 40.56 | C |
| ATOM | 7416 | CB | VAL | C | 191 | −60.334 | −22.778 | 67.565 | 1.00 | 41.54 | C |
| ATOM | 7417 | CG1 | VAL | C | 191 | −59.490 | −21.557 | 67.225 | 1.00 | 49.83 | C |
| ATOM | 7418 | CG2 | VAL | C | 191 | −60.558 | −23.622 | 66.325 | 1.00 | 39.50 | C |
| ATOM | 7419 | C | VAL | C | 191 | −59.493 | −22.748 | 69.923 | 1.00 | 43.00 | C |
| ATOM | 7420 | O | VAL | C | 191 | −58.437 | −22.213 | 70.160 | 1.00 | 45.85 | O |
| ATOM | 7421 | N | THR | C | 192 | −60.506 | −22.637 | 70.755 | 1.00 | 44.95 | N |
| ATOM | 7422 | CA | THR | C | 192 | −60.358 | −21.963 | 72.031 | 1.00 | 47.49 | C |
| ATOM | 7423 | CB | THR | C | 192 | −61.708 | −21.827 | 72.765 | 1.00 | 51.36 | C |
| ATOM | 7424 | OG1 | THR | C | 192 | −62.598 | −21.033 | 71.971 | 1.00 | 49.91 | O |
| ATOM | 7425 | CG2 | THR | C | 192 | −61.519 | −21.156 | 74.120 | 1.00 | 52.99 | C |
| ATOM | 7426 | C | THR | C | 192 | −59.320 | −22.617 | 72.924 | 1.00 | 49.07 | C |
| ATOM | 7427 | O | THR | C | 192 | −58.441 | −21.975 | 73.436 | 1.00 | 51.83 | O |
| ATOM | 7428 | N | ALA | C | 193 | −59.443 | −23.912 | 73.125 | 1.00 | 43.93 | N |
| ATOM | 7429 | CA | ALA | C | 193 | −58.526 | −24.660 | 73.960 | 1.00 | 40.90 | C |
| ATOM | 7430 | CB | ALA | C | 193 | −58.959 | −26.117 | 74.033 | 1.00 | 37.59 | C |
| ATOM | 7431 | C | ALA | C | 193 | −57.084 | −24.561 | 73.493 | 1.00 | 45.02 | C |
| ATOM | 7432 | O | ALA | C | 193 | −56.193 | −24.362 | 74.296 | 1.00 | 44.38 | O |
| ATOM | 7433 | N | ALA | C | 194 | −56.893 | −24.621 | 72.166 | 1.00 | 49.09 | N |
| ATOM | 7434 | CA | ALA | C | 194 | −55.599 | −24.406 | 71.516 | 1.00 | 38.90 | C |
| ATOM | 7435 | CB | ALA | C | 194 | −55.694 | −24.716 | 70.028 | 1.00 | 34.60 | C |
| ATOM | 7436 | C | ALA | C | 194 | −55.068 | −22.999 | 71.737 | 1.00 | 44.72 | C |
| ATOM | 7437 | O | ALA | C | 194 | −53.947 | −22.800 | 72.138 | 1.00 | 42.97 | O |
| ATOM | 7438 | N | TYR | C | 195 | −55.912 | −22.015 | 71.483 | 1.00 | 44.94 | N |
| ATOM | 7439 | CA | TYR | C | 195 | −55.580 | −20.617 | 71.700 | 1.00 | 48.14 | C |
| ATOM | 7440 | CB | TYR | C | 195 | −56.756 | −19.711 | 71.330 | 1.00 | 48.24 | C |
| ATOM | 7441 | CG | TYR | C | 195 | −56.412 | −18.239 | 71.270 | 1.00 | 49.78 | C |
| ATOM | 7442 | CD1 | TYR | C | 195 | −55.349 | −17.786 | 70.505 | 1.00 | 52.57 | C |
| ATOM | 7443 | CE1 | TYR | C | 195 | −55.040 | −16.444 | 70.439 | 1.00 | 53.51 | C |
| ATOM | 7444 | CZ | TYR | C | 195 | −55.800 | −15.533 | 71.136 | 1.00 | 48.30 | C |
| ATOM | 7445 | OH | TYR | C | 195 | −55.488 | −14.195 | 71.065 | 1.00 | 47.92 | O |
| ATOM | 7446 | CE2 | TYR | C | 195 | −56.865 | −15.954 | 71.899 | 1.00 | 47.99 | C |
| ATOM | 7447 | CD2 | TYR | C | 195 | −57.167 | −17.301 | 71.961 | 1.00 | 49.48 | C |
| ATOM | 7448 | C | TYR | C | 195 | −55.102 | −20.334 | 73.127 | 1.00 | 45.85 | C |
| ATOM | 7449 | O | TYR | C | 195 | −54.080 | −19.731 | 73.336 | 1.00 | 46.17 | O |
| ATOM | 7450 | N | ASN | C | 196 | −55.820 | −20.864 | 74.088 | 1.00 | 41.48 | N |
| ATOM | 7451 | CA | ASN | C | 196 | −55.557 | −20.664 | 75.494 | 1.00 | 40.09 | C |
| ATOM | 7452 | CB | ASN | C | 196 | −56.739 | −21.124 | 76.341 | 1.00 | 41.61 | C |
| ATOM | 7453 | CG | ASN | C | 196 | −57.902 | −20.164 | 76.264 | 1.00 | 46.40 | C |
| ATOM | 7454 | OD1 | ASN | C | 196 | −57.723 | −18.982 | 75.968 | 1.00 | 44.25 | O |
| ATOM | 7455 | ND2 | ASN | C | 196 | −59.102 | −20.662 | 76.526 | 1.00 | 55.83 | N |
| ATOM | 7456 | C | ASN | C | 196 | −54.283 | −21.358 | 75.906 | 1.00 | 40.83 | C |
| ATOM | 7457 | O | ASN | C | 196 | −53.496 | −20.822 | 76.639 | 1.00 | 38.71 | O |
| ATOM | 7458 | N | TYR | C | 197 | −54.055 | −22.548 | 75.382 | 1.00 | 41.19 | N |
| ATOM | 7459 | CA | TYR | C | 197 | −52.810 | −23.258 | 75.633 | 1.00 | 40.06 | C |
| ATOM | 7460 | CB | TYR | C | 197 | −52.865 | −24.645 | 74.975 | 1.00 | 37.04 | C |
| ATOM | 7461 | CG | TYR | C | 197 | −51.515 | −25.305 | 74.751 | 1.00 | 40.23 | C |
| ATOM | 7462 | CD1 | TYR | C | 197 | −50.919 | −26.085 | 75.738 | 1.00 | 33.79 | C |
| ATOM | 7463 | CE1 | TYR | C | 197 | −49.685 | −26.682 | 75.528 | 1.00 | 32.13 | C |
| ATOM | 7464 | CZ | TYR | C | 197 | −49.037 | −26.503 | 74.324 | 1.00 | 34.66 | C |
| ATOM | 7465 | OH | TYR | C | 197 | −47.810 | −27.087 | 74.102 | 1.00 | 36.60 | O |
| ATOM | 7466 | CE2 | TYR | C | 197 | −49.612 | −25.739 | 73.331 | 1.00 | 36.01 | C |
| ATOM | 7467 | CD2 | TYR | C | 197 | −50.842 | −25.152 | 73.545 | 1.00 | 41.78 | C |
| ATOM | 7468 | C | TYR | C | 197 | −51.601 | −22.477 | 75.132 | 1.00 | 45.59 | C |
| ATOM | 7469 | O | TYR | C | 197 | −50.607 | −22.376 | 75.805 | 1.00 | 40.98 | O |
| ATOM | 7470 | N | PHE | C | 198 | −51.742 | −21.840 | 73.989 | 1.00 | 47.77 | N |
| ATOM | 7471 | CA | PHE | C | 198 | −50.745 | −20.923 | 73.457 | 1.00 | 42.54 | C |
| ATOM | 7472 | CB | PHE | C | 198 | −51.081 | −20.461 | 72.044 | 1.00 | 41.22 | C |
| ATOM | 7473 | CG | PHE | C | 198 | −50.902 | −21.545 | 71.026 | 1.00 | 42.55 | C |
| ATOM | 7474 | CD1 | PHE | C | 198 | −49.960 | −22.541 | 71.233 | 1.00 | 42.79 | C |
| ATOM | 7475 | CE1 | PHE | C | 198 | −49.788 | −23.557 | 70.318 | 1.00 | 42.91 | C |
| ATOM | 7476 | CZ | PHE | C | 198 | −50.564 | −23.596 | 69.184 | 1.00 | 47.74 | C |
| ATOM | 7477 | CE2 | PHE | C | 198 | −51.513 | −22.617 | 68.969 | 1.00 | 49.01 | C |
| ATOM | 7478 | CD2 | PHE | C | 198 | −51.682 | −21.598 | 69.891 | 1.00 | 47.06 | C |
| ATOM | 7479 | C | PHE | C | 198 | −50.420 | −19.818 | 74.405 | 1.00 | 45.76 | C |
| ATOM | 7480 | O | PHE | C | 198 | −49.280 | −19.489 | 74.590 | 1.00 | 51.12 | O |
| ATOM | 7481 | N | HIS | C | 199 | −51.436 | −19.254 | 75.011 | 1.00 | 42.99 | N |
| ATOM | 7482 | CA | HIS | C | 199 | −51.267 | −18.195 | 75.961 | 1.00 | 45.37 | C |
| ATOM | 7483 | CB | HIS | C | 199 | −52.643 | −17.698 | 76.372 | 1.00 | 44.56 | C |
| ATOM | 7484 | CG | HIS | C | 199 | −52.659 | −16.268 | 76.785 | 1.00 | 47.28 | C |
| ATOM | 7485 | ND1 | HIS | C | 199 | −53.828 | −15.559 | 76.954 | 1.00 | 56.52 | N |
| ATOM | 7486 | CE1 | HIS | C | 199 | −53.537 | −14.323 | 77.313 | 1.00 | 60.75 | C |
| ATOM | 7487 | NE2 | HIS | C | 199 | −52.223 | −14.204 | 77.381 | 1.00 | 56.44 | N |
| ATOM | 7488 | CD2 | HIS | C | 199 | −51.651 | −15.408 | 77.052 | 1.00 | 50.74 | C |
| ATOM | 7489 | C | HIS | C | 199 | −50.520 | −18.652 | 77.221 | 1.00 | 46.53 | C |
| ATOM | 7490 | O | HIS | C | 199 | −49.653 | −17.976 | 77.736 | 1.00 | 49.67 | O |
| ATOM | 7491 | N | VAL | C | 200 | −50.835 | −19.852 | 77.687 | 1.00 | 44.19 | N |
| ATOM | 7492 | CA | VAL | C | 200 | −50.135 | −20.465 | 78.800 | 1.00 | 44.40 | C |
| ATOM | 7493 | CB | VAL | C | 200 | −50.772 | −21.802 | 79.234 | 1.00 | 42.44 | C |
| ATOM | 7494 | CG1 | VAL | C | 200 | −50.128 | −22.287 | 80.515 | 1.00 | 36.30 | C |
| ATOM | 7495 | CG2 | VAL | C | 200 | −52.266 | −21.640 | 79.442 | 1.00 | 42.60 | C |
| ATOM | 7496 | C | VAL | C | 200 | −48.695 | −20.684 | 78.462 | 1.00 | 44.32 | C |
| ATOM | 7497 | O | VAL | C | 200 | −47.818 | −20.396 | 79.232 | 1.00 | 47.24 | O |
| ATOM | 7498 | N | THR | C | 201 | −48.475 | −21.143 | 77.250 | 1.00 | 45.12 | N |
| ATOM | 7499 | CA | THR | C | 201 | −47.159 | −21.373 | 76.705 | 1.00 | 44.34 | C |
| ATOM | 7500 | CB | THR | C | 201 | −47.240 | −21.881 | 75.256 | 1.00 | 40.13 | C |
| ATOM | 7501 | OG1 | THR | C | 201 | −48.188 | −22.952 | 75.174 | 1.00 | 46.46 | O |
| ATOM | 7502 | CG2 | THR | C | 201 | −45.899 | −22.387 | 74.808 | 1.00 | 45.12 | C |
| ATOM | 7503 | C | THR | C | 201 | −46.327 | −20.102 | 76.722 | 1.00 | 44.86 | C |
| ATOM | 7504 | O | THR | C | 201 | −45.209 | −20.112 | 77.148 | 1.00 | 43.86 | O |
| ATOM | 7505 | N | ASN | C | 202 | −46.881 | −18.997 | 76.267 | 1.00 | 45.05 | N |
| ATOM | 7506 | CA | ASN | C | 202 | −46.195 | −17.712 | 76.300 | 1.00 | 44.41 | C |
| ATOM | 7507 | CB | ASN | C | 202 | −47.127 | −16.604 | 75.806 | 1.00 | 44.17 | C |
| ATOM | 7508 | CG | ASN | C | 202 | −47.450 | −16.729 | 74.332 | 1.00 | 45.80 | C |
| ATOM | 7509 | OD1 | ASN | C | 202 | −47.209 | −17.770 | 73.719 | 1.00 | 45.42 | O |
| ATOM | 7510 | ND2 | ASN | C | 202 | −47.998 | −15.666 | 73.754 | 1.00 | 46.00 | N |
| ATOM | 7511 | C | ASN | C | 202 | −45.676 | −17.352 | 77.692 | 1.00 | 48.19 | C |
| ATOM | 7512 | O | ASN | C | 202 | −44.524 | −17.025 | 77.863 | 1.00 | 55.49 | O |
| ATOM | 7513 | N | PHE | C | 203 | −46.523 | −17.466 | 78.693 | 1.00 | 44.24 | N |
| ATOM | 7514 | CA | PHE | C | 203 | −46.143 | −17.156 | 80.058 | 1.00 | 44.16 | C |
| ATOM | 7515 | CB | PHE | C | 203 | −47.359 | −17.203 | 80.979 | 1.00 | 48.05 | C |
| ATOM | 7516 | CG | PHE | C | 203 | −48.116 | −15.908 | 81.043 | 1.00 | 54.18 | C |
| ATOM | 7517 | CD2 | PHE | C | 203 | −48.245 | −15.227 | 82.241 | 1.00 | 52.48 | C |
| ATOM | 7518 | CE2 | PHE | C | 203 | −48.944 | −14.038 | 82.304 | 1.00 | 54.82 | C |
| ATOM | 7519 | CZ | PHE | C | 203 | −49.523 | −13.514 | 81.162 | 1.00 | 53.18 | C |
| ATOM | 7520 | CE1 | PHE | C | 203 | −49.401 | −14.179 | 79.963 | 1.00 | 46.22 | C |
| ATOM | 7521 | CD1 | PHE | C | 203 | −48.702 | −15.371 | 79.906 | 1.00 | 54.73 | C |
| ATOM | 7522 | C | PHE | C | 203 | −45.059 | −18.088 | 80.547 | 1.00 | 43.12 | C |
| ATOM | 7523 | O | PHE | C | 203 | −44.066 | −17.674 | 81.093 | 1.00 | 44.33 | O |
| ATOM | 7524 | N | PHE | C | 204 | −45.281 | −19.363 | 80.339 | 1.00 | 40.57 | N |
| ATOM | 7525 | CA | PHE | C | 204 | −44.353 | −20.419 | 80.697 | 1.00 | 44.12 | C |
| ATOM | 7526 | CB | PHE | C | 204 | −45.004 | −21.792 | 80.541 | 1.00 | 42.20 | C |
| ATOM | 7527 | CG | PHE | C | 204 | −45.920 | −22.155 | 81.682 | 1.00 | 39.68 | C |
| ATOM | 7528 | CD2 | PHE | C | 204 | −45.751 | −23.342 | 82.370 | 1.00 | 38.18 | C |
| ATOM | 7529 | CE2 | PHE | C | 204 | −46.592 | −23.679 | 83.417 | 1.00 | 33.06 | C |
| ATOM | 7530 | CZ | PHE | C | 204 | −47.611 | −22.826 | 83.790 | 1.00 | 31.80 | C |
| ATOM | 7531 | CE1 | PHE | C | 204 | −47.792 | −21.641 | 83.115 | 1.00 | 37.00 | C |
| ATOM | 7532 | CD1 | PHE | C | 204 | −46.951 | −21.308 | 82.066 | 1.00 | 38.97 | C |
| ATOM | 7533 | C | PHE | C | 204 | −42.965 | −20.320 | 79.995 | 1.00 | 48.19 | C |
| ATOM | 7534 | O | PHE | C | 204 | −41.942 | −20.437 | 80.629 | 1.00 | 45.53 | O |
| ATOM | 7535 | N | TRP | C | 205 | −42.925 | −19.988 | 78.715 | 1.00 | 44.26 | N |
| ATOM | 7536 | CA | TRP | C | 205 | −41.669 | −19.659 | 78.045 | 1.00 | 46.98 | C |
| ATOM | 7537 | CB | TRP | C | 205 | −41.774 | −19.678 | 76.515 | 1.00 | 43.40 | C |
| ATOM | 7538 | CG | TRP | C | 205 | −41.623 | −21.079 | 76.008 | 1.00 | 42.42 | C |
| ATOM | 7539 | CD1 | TRP | C | 205 | −42.582 | −21.843 | 75.416 | 1.00 | 39.33 | C |
| ATOM | 7540 | NE1 | TRP | C | 205 | −42.085 | −23.088 | 75.125 | 1.00 | 39.11 | N |
| ATOM | 7541 | CE2 | TRP | C | 205 | −40.784 | −23.158 | 75.547 | 1.00 | 48.08 | C |
| ATOM | 7542 | CD2 | TRP | C | 205 | −40.458 | −21.912 | 76.119 | 1.00 | 50.19 | C |
| ATOM | 7543 | CE3 | TRP | C | 205 | −39.169 | −21.722 | 76.630 | 1.00 | 46.74 | C |
| ATOM | 7544 | CZ3 | TRP | C | 205 | −38.267 | −22.770 | 76.555 | 1.00 | 49.40 | C |
| ATOM | 7545 | CH2 | TRP | C | 205 | −38.629 | −23.997 | 75.979 | 1.00 | 48.45 | C |
| ATOM | 7546 | CZ2 | TRP | C | 205 | −39.878 | −24.207 | 75.474 | 1.00 | 46.28 | C |
| ATOM | 7547 | C | TRP | C | 205 | −40.981 | −18.400 | 78.595 | 1.00 | 49.27 | C |
| ATOM | 7548 | O | TRP | C | 205 | −39.788 | −18.377 | 78.812 | 1.00 | 50.74 | O |
| ATOM | 7549 | N | MET | C | 206 | −41.729 | −17.348 | 78.860 | 1.00 | 46.39 | N |
| ATOM | 7550 | CA | MET | C | 206 | −41.163 | −16.187 | 79.523 | 1.00 | 47.21 | C |
| ATOM | 7551 | CB | MET | C | 206 | −42.235 | −15.118 | 79.750 | 1.00 | 54.60 | C |
| ATOM | 7552 | CG | MET | C | 206 | −42.616 | −14.325 | 78.511 | 1.00 | 52.87 | C |
| ATOM | 7553 | SD | MET | C | 206 | −41.242 | −13.354 | 77.861 | 1.00 | 59.80 | S |
| ATOM | 7554 | CE | MET | C | 206 | −42.079 | −12.390 | 76.604 | 1.00 | 50.30 | C |
| ATOM | 7555 | C | MET | C | 206 | −40.524 | −16.562 | 80.859 | 1.00 | 47.61 | C |
| ATOM | 7556 | O | MET | C | 206 | −39.429 | −16.174 | 81.160 | 1.00 | 50.35 | O |
| ATOM | 7557 | N | PHE | C | 207 | −41.192 | −17.395 | 81.626 | 1.00 | 48.06 | N |
| ATOM | 7558 | CA | PHE | C | 207 | −40.627 | −17.967 | 82.851 | 1.00 | 50.62 | C |
| ATOM | 7559 | CB | PHE | C | 207 | −41.713 | −18.776 | 83.543 | 1.00 | 44.71 | C |
| ATOM | 7560 | CG | PHE | C | 207 | −41.252 | −19.528 | 84.752 | 1.00 | 47.38 | C |
| ATOM | 7561 | CD1 | PHE | C | 207 | −41.106 | −18.887 | 85.969 | 1.00 | 57.95 | C |
| ATOM | 7562 | CE1 | PHE | C | 207 | −40.700 | −19.588 | 87.091 | 1.00 | 62.16 | C |
| ATOM | 7563 | CZ | PHE | C | 207 | −40.456 | −20.947 | 87.003 | 1.00 | 55.28 | C |
| ATOM | 7564 | CE2 | PHE | C | 207 | −40.608 | −21.597 | 85.794 | 1.00 | 45.78 | C |
| ATOM | 7565 | CD2 | PHE | C | 207 | −41.00 | −20.890 | 84.681 | 1.00 | 48.65 | C |
| ATOM | 7566 | C | PHE | C | 207 | −39.412 | −18.891 | 82.582 | 1.00 | 52.71 | C |
| ATOM | 7567 | O | PHE | C | 207 | −38.520 | −19.030 | 83.396 | 1.00 | 54.27 | O |
| ATOM | 7568 | N | GLY | C | 208 | −39.337 | −19.452 | 81.390 | 1.00 | 50.05 | N |
| ATOM | 7569 | CA | GLY | C | 208 | −38.186 | −20.176 | 80.948 | 1.00 | 49.41 | C |
| ATOM | 7570 | C | GLY | C | 208 | −36.987 | −19.276 | 80.835 | 1.00 | 54.93 | C |
| ATOM | 7571 | O | GLY | C | 208 | −35.910 | −19.614 | 81.303 | 1.00 | 59.80 | O |
| ATOM | 7572 | N | GLU | C | 209 | −37.200 | −18.077 | 80.272 | 1.00 | 56.38 | N |
| ATOM | 7573 | CA | GLU | C | 209 | −36.179 | −17.039 | 80.234 | 1.00 | 51.48 | C |
| ATOM | 7574 | CB | GLU | C | 209 | −36.688 | −15.813 | 79.472 | 1.00 | 48.84 | C |
| ATOM | 7575 | CG | GLU | C | 209 | −36.952 | −16.047 | 77.999 | 1.00 | 55.26 | C |
| ATOM | 7576 | CD | GLU | C | 209 | −35.683 | −16.065 | 77.184 | 1.00 | 56.66 | C |
| ATOM | 7577 | OE1 | GLU | C | 209 | −34.674 | −15.487 | 77.642 | 1.00 | 66.36 | O |
| ATOM | 7578 | OE2 | GLU | C | 209 | −35.693 | −16.659 | 76.087 | 1.00 | 53.42 | O |
| ATOM | 7579 | C | GLU | C | 209 | −35.774 | −16.613 | 81.632 | 1.00 | 57.44 | C |
| ATOM | 7580 | O | GLU | C | 209 | −34.615 | −16.432 | 81.935 | 1.00 | 63.35 | O |
| ATOM | 7581 | N | GLY | C | 210 | −36.750 | −16.437 | 82.495 | 1.00 | 57.46 | N |
| ATOM | 7582 | CA | GLY | C | 210 | −36.480 | −16.025 | 83.826 | 1.00 | 59.30 | C |
| ATOM | 7583 | C | GLY | C | 210 | −35.628 | −16.991 | 84.571 | 1.00 | 61.49 | C |
| ATOM | 7584 | O | GLY | C | 210 | −34.710 | −16.570 | 85.248 | 1.00 | 65.09 | O |
| ATOM | 7585 | N | CYS | C | 211 | −35.919 | −18.288 | 84.445 | 1.00 | 58.00 | N |
| ATOM | 7586 | CA | CYS | C | 211 | −35.169 | −19.329 | 85.138 | 1.00 | 59.80 | C |
| ATOM | 7587 | CB | CYS | C | 211 | −35.851 | −20.685 | 85.002 | 1.00 | 59.33 | C |
| ATOM | 7588 | SG | CYS | C | 211 | −37.334 | −20.804 | 85.988 | 1.00 | 58.19 | S |
| ATOM | 7589 | C | CYS | C | 211 | −33.719 | −19.414 | 84.661 | 1.00 | 58.21 | C |
| ATOM | 7590 | O | CYS | C | 211 | −32.815 | −19.549 | 85.459 | 1.00 | 60.25 | O |
| ATOM | 7591 | N | TYR | C | 212 | −33.487 | −19.315 | 83.354 | 1.00 | 57.50 | N |
| ATOM | 7592 | CA | TYR | C | 212 | −32.112 | −19.261 | 82.877 | 1.00 | 61.48 | C |
| ATOM | 7593 | CB | TYR | C | 212 | −32.066 | −19.267 | 81.348 | 1.00 | 60.88 | C |
| ATOM | 7594 | CG | TYR | C | 212 | −30.677 | −19.468 | 80.782 | 1.00 | 67.62 | C |
| ATOM | 7595 | CD2 | TYR | C | 212 | −30.211 | −20.741 | 80.484 | 1.00 | 72.40 | C |
| ATOM | 7596 | CE2 | TYR | C | 212 | −28.949 | −20.934 | 79.962 | 1.00 | 78.74 | C |
| ATOM | 7597 | CZ | TYR | C | 212 | −28.132 | −19.846 | 79.731 | 1.00 | 78.05 | C |
| ATOM | 7598 | OH | TYR | C | 212 | −26.872 | −20.036 | 79.211 | 1.00 | 87.90 | O |
| ATOM | 7599 | CE1 | TYR | C | 212 | −28.570 | −18.571 | 80.017 | 1.00 | 69.55 | C |
| ATOM | 7600 | CD1 | TYR | C | 212 | −29.836 | −18.387 | 80.538 | 1.00 | 65.26 | C |
| ATOM | 7601 | C | TYR | C | 212 | −31.380 | −18.024 | 83.405 | 1.00 | 64.57 | C |
| ATOM | 7602 | O | TYR | C | 212 | −30.308 | −18.123 | 83.968 | 1.00 | 68.82 | O |
| ATOM | 7603 | N | LEU | C | 213 | −31.988 | −16.853 | 83.208 | 1.00 | 67.47 | N |
| ATOM | 7604 | CA | LEU | C | 213 | −31.388 | −15.581 | 83.599 | 1.00 | 71.37 | C |
| ATOM | 7605 | CB | LEU | C | 213 | −32.330 | −14.425 | 83.258 | 1.00 | 68.82 | C |
| ATOM | 7606 | CG | LEU | C | 213 | −31.754 | −13.021 | 83.433 | 1.00 | 71.30 | C |
| ATOM | 7607 | CD1 | LEU | C | 213 | −30.512 | −12.854 | 82.571 | 1.00 | 85.02 | C |
| ATOM | 7608 | CD2 | LEU | C | 213 | −32.796 | −11.968 | 83.092 | 1.00 | 69.02 | C |
| ATOM | 7609 | C | LEU | C | 213 | −31.024 | −15.534 | 85.074 | 1.00 | 76.63 | C |
| ATOM | 7610 | O | LEU | C | 213 | −29.882 | −15.303 | 85.437 | 1.00 | 85.68 | O |
| ATOM | 7611 | N | HIS | C | 214 | −31.991 | −15.860 | 85.919 | 1.00 | 69.81 | N |
| ATOM | 7612 | CA | HIS | C | 214 | −31.754 | −15.919 | 87.340 | 1.00 | 69.56 | C |
| ATOM | 7613 | CB | HIS | C | 214 | −32.999 | −16.447 | 88.058 | 1.00 | 71.24 | C |
| ATOM | 7614 | CG | HIS | C | 214 | −32.816 | −16.617 | 89.534 | 1.00 | 73.79 | C |
| ATOM | 7615 | ND1 | HIS | C | 214 | −32.297 | −17.764 | 90.091 | 1.00 | 77.04 | N |
| ATOM | 7616 | CE1 | HIS | C | 214 | −32.250 | −17.629 | 91.405 | 1.00 | 78.16 | C |
| ATOM | 7617 | NE2 | HIS | C | 214 | −32.721 | −16.437 | 91.718 | 1.00 | 77.52 | N |
| ATOM | 7618 | CD2 | HIS | C | 214 | −33.083 | −15.782 | 90.565 | 1.00 | 73.00 | C |
| ATOM | 7619 | C | HIS | C | 214 | −30.544 | −16.763 | 87.717 | 1.00 | 77.19 | C |
| ATOM | 7620 | O | HIS | C | 214 | −29.654 | −16.256 | 88.342 | 1.00 | 85.54 | O |
| ATOM | 7621 | N | THR | C | 215 | −30.522 | −18.060 | 87.386 | 1.00 | 74.06 | N |
| ATOM | 7622 | CA | THR | C | 215 | −29.385 | −18.902 | 87.777 | 1.00 | 77.22 | C |
| ATOM | 7623 | CB | THR | C | 215 | −29.625 | −20.391 | 87.458 | 1.00 | 71.69 | C |
| ATOM | 7624 | OG1 | THR | C | 215 | −29.655 | −20.584 | 86.039 | 1.00 | 77.63 | O |
| ATOM | 7625 | CG2 | THR | C | 215 | −30.937 | −20.862 | 88.066 | 1.00 | 69.76 | C |
| ATOM | 7626 | C | THR | C | 215 | −28.053 | −18.450 | 87.156 | 1.00 | 79.36 | C |
| ATOM | 7627 | O | THR | C | 215 | −27.002 | −18.497 | 87.791 | 1.00 | 91.29 | O |
| ATOM | 7628 | N | ALA | C | 216 | −28.090 | −18.069 | 85.887 | 1.00 | 73.61 | N |
| ATOM | 7629 | CA | ALA | C | 216 | −26.876 | −17.718 | 85.205 | 1.00 | 78.92 | C |
| ATOM | 7630 | CB | ALA | C | 216 | −27.164 | −17.351 | 83.756 | 1.00 | 78.21 | C |
| ATOM | 7631 | C | ALA | C | 216 | −26.151 | −16.582 | 85.905 | 1.00 | 90.33 | C |
| ATOM | 7632 | O | ALA | C | 216 | −24.938 | −16.620 | 86.067 | 1.00 | 104.68 | O |
| ATOM | 7633 | N | ILE | C | 217 | −26.899 | −15.603 | 86.396 | 1.00 | 84.17 | N |
| ATOM | 7634 | CA | ILE | C | 217 | −26.266 | −14.541 | 87.147 | 1.00 | 84.85 | C |
| ATOM | 7635 | CB | ILE | C | 217 | −27.144 | −13.283 | 87.142 | 1.00 | 81.01 | C |
| ATOM | 7636 | CG1 | ILE | C | 217 | −27.664 | −12.999 | 85.733 | 1.00 | 86.45 | C |
| ATOM | 7637 | CD1 | ILE | C | 217 | −28.839 | −12.042 | 85.709 | 1.00 | 88.43 | C |
| ATOM | 7638 | CG2 | ILE | C | 217 | −26.385 | −12.091 | 87.709 | 1.00 | 82.97 | C |
| ATOM | 7639 | C | ILE | C | 217 | −26.058 | −14.937 | 88.623 | 1.00 | 86.04 | C |
| ATOM | 7640 | O | ILE | C | 217 | −25.020 | −14.704 | 89.227 | 1.00 | 90.86 | O |
| ATOM | 7641 | O | VAL | C | 218 | −25.713 | −16.700 | 92.196 | 1.00 | 95.88 | O |
| ATOM | 7642 | N | VAL | C | 218 | −27.134 | −15.438 | 89.212 | 1.00 | 85.59 | N |
| ATOM | 7643 | CA | VAL | C | 218 | −27.213 | −15.695 | 90.638 | 1.00 | 89.76 | C |
| ATOM | 7644 | C | VAL | C | 218 | −26.342 | −16.834 | 91.155 | 1.00 | 94.54 | C |
| ATOM | 7645 | CB | VAL | C | 218 | −28.681 | −15.859 | 91.132 | 1.00 | 82.81 | C |
| ATOM | 7646 | CG1 | VAL | C | 218 | −28.738 | −16.389 | 92.562 | 1.00 | 92.41 | C |
| ATOM | 7647 | CG2 | VAL | C | 218 | −29.422 | −14.536 | 91.019 | 1.00 | 80.15 | C |
| ATOM | 7648 | O | LEU | C | 219 | −23.550 | −19.666 | 91.389 | 1.00 | 118.54 | O |
| ATOM | 7649 | N | LEU | C | 219 | −26.489 | −18.019 | 90.576 | 1.00 | 95.23 | N |
| ATOM | 7650 | CA | LEU | C | 219 | −25.886 | −19.179 | 91.203 | 1.00 | 96.03 | C |
| ATOM | 7651 | C | LEU | C | 219 | −24.500 | −19.482 | 90.618 | 1.00 | 102.86 | C |
| ATOM | 7652 | CB | LEU | C | 219 | −26.801 | −20.399 | 91.066 | 1.00 | 102.70 | C |
| ATOM | 7653 | CG | LEU | C | 219 | −28.188 | −20.287 | 91.709 | 1.00 | 100.78 | C |
| ATOM | 7654 | CD1 | LEU | C | 219 | −29.025 | −21.528 | 91.418 | 1.00 | 100.79 | C |
| ATOM | 7655 | CD2 | LEU | C | 219 | −28.079 | −20.046 | 93.211 | 1.00 | 98.67 | C |
| ATOM | 7656 | O | THR | C | 220 | −21.057 | −18.698 | 90.079 | 1.00 | 98.52 | O |
| ATOM | 7657 | N | THR | C | 220 | −24.339 | −19.294 | 89.295 | 1.00 | 93.24 | N |
| ATOM | 7658 | CA | THR | C | 220 | −22.994 | −19.290 | 88.702 | 1.00 | 97.14 | C |
| ATOM | 7659 | C | THR | C | 220 | −22.015 | −18.288 | 89.369 | 1.00 | 99.65 | C |
| ATOM | 7660 | CB | THR | C | 220 | −23.042 | −19.032 | 87.186 | 1.00 | 100.63 | C |
| ATOM | 7661 | OG1 | THR | C | 220 | −24.018 | −19.893 | 86.586 | 1.00 | 103.43 | O |
| ATOM | 7662 | CG2 | THR | C | 220 | −21.680 | −19.294 | 86.555 | 1.00 | 102.88 | C |
| ATOM | 7663 | O | ASP | C | 224 | −25.830 | −27.592 | 84.564 | 1.00 | 111.68 | O |
| ATOM | 7664 | N | ASP | C | 224 | −24.159 | −25.855 | 86.376 | 1.00 | 104.06 | N |
| ATOM | 7665 | CA | ASP | C | 224 | −25.643 | −25.787 | 86.196 | 1.00 | 113.68 | C |
| ATOM | 7666 | C | ASP | C | 224 | −26.261 | −27.053 | 85.586 | 1.00 | 113.78 | C |
| ATOM | 7667 | CB | ASP | C | 224 | −26.073 | −24.528 | 85.430 | 1.00 | 109.19 | C |
| ATOM | 7668 | CG | ASP | C | 224 | −25.536 | −24.486 | 84.016 | 1.00 | 116.09 | C |
| ATOM | 7669 | OD1 | ASP | C | 224 | −24.575 | −25.226 | 83.712 | 1.00 | 125.60 | O |
| ATOM | 7670 | OD2 | ASP | C | 224 | −26.075 | −23.695 | 83.211 | 1.00 | 107.59 | O |
| ATOM | 7671 | O | ARG | C | 225 | −29.297 | −29.635 | 84.197 | 1.00 | 102.34 | O |
| ATOM | 7672 | N | ARG | C | 225 | −27.253 | −27.547 | 86.313 | 1.00 | 109.02 | N |
| ATOM | 7673 | CA | ARG | C | 225 | −27.945 | −28.794 | 86.016 | 1.00 | 110.75 | C |
| ATOM | 7674 | C | ARG | C | 225 | −28.867 | −28.652 | 84.797 | 1.00 | 106.53 | C |
| ATOM | 7675 | CB | ARG | C | 225 | −28.745 | −29.207 | 87.264 | 1.00 | 117.50 | C |
| ATOM | 7676 | CG | ARG | C | 225 | −29.914 | −30.161 | 87.051 | 1.00 | 117.52 | C |
| ATOM | 7677 | CD | ARG | C | 225 | −30.754 | −30.275 | 88.314 | 1.00 | 120.37 | C |
| ATOM | 7678 | NE | ARG | C | 225 | −31.200 | −28.964 | 88.785 | 1.00 | 127.84 | N |
| ATOM | 7679 | CZ | ARG | C | 225 | −31.964 | −28.773 | 89.856 | 1.00 | 140.48 | C |
| ATOM | 7680 | NH1 | ARG | C | 225 | −32.371 | −29.810 | 90.574 | 1.00 | 143.57 | N |
| ATOM | 7681 | NH2 | ARG | C | 225 | −32.323 | −27.545 | 90.212 | 1.00 | 140.22 | N |
| ATOM | 7682 | O | LEU | C | 226 | −28.826 | −27.215 | 81.809 | 1.00 | 96.67 | O |
| ATOM | 7683 | N | LEU | C | 226 | −29.288 | −27.401 | 84.572 | 1.00 | 106.74 | N |
| ATOM | 7684 | CA | LEU | C | 226 | −30.334 | −27.076 | 83.623 | 1.00 | 95.30 | C |
| ATOM | 7685 | C | LEU | C | 226 | −29.951 | −27.464 | 82.213 | 1.00 | 92.93 | C |
| ATOM | 7686 | CB | LEU | C | 226 | −30.647 | −25.577 | 83.667 | 1.00 | 78.52 | C |
| ATOM | 7687 | CG | LEU | C | 226 | −31.100 | −24.971 | 84.996 | 1.00 | 79.29 | C |
| ATOM | 7688 | CD1 | LEU | C | 226 | −31.301 | −23.473 | 84.847 | 1.00 | 67.26 | C |
| ATOM | 7689 | CD2 | LEU | C | 226 | −32.374 | −25.635 | 85.500 | 1.00 | 89.26 | C |
| ATOM | 7690 | O | ARG | C | 227 | −33.019 | −27.693 | 79.734 | 1.00 | 71.41 | O |
| ATOM | 7691 | N | ARG | C | 227 | −30.934 | −27.955 | 81.433 | 1.00 | 82.82 | N |
| ATOM | 7692 | CA | ARG | C | 227 | −30.675 | −28.205 | 80.039 | 1.00 | 74.15 | C |
| ATOM | 7693 | C | ARG | C | 227 | −31.956 | −28.074 | 79.233 | 1.00 | 75.62 | C |
| ATOM | 7694 | CB | ARG | C | 227 | −30.180 | −29.645 | 79.817 | 1.00 | 73.53 | C |
| ATOM | 7695 | CG | ARG | C | 227 | −29.187 | −30.219 | 80.821 | 1.00 | 86.66 | C |
| ATOM | 7696 | CD | ARG | C | 227 | −27.759 | −29.829 | 80.499 | 1.00 | 91.65 | C |
| ATOM | 7697 | NE | ARG | C | 227 | −26.810 | −30.489 | 81.388 | 1.00 | 80.83 | N |
| ATOM | 7698 | CZ | ARG | C | 227 | −25.497 | −30.307 | 81.337 | 1.00 | 88.21 | C |
| ATOM | 7699 | NH1 | ARG | C | 227 | −24.980 | −29.471 | 80.446 | 1.00 | 84.63 | N |
| ATOM | 7700 | NH2 | ARG | C | 227 | −24.703 | −30.957 | 82.177 | 1.00 | 97.69 | N |
| ATOM | 7701 | N | ALA | C | 228 | −31.807 | −28.360 | 77.939 | 1.00 | 70.17 | N |
| ATOM | 7702 | CA | ALA | C | 228 | −32.851 | −28.131 | 76.979 | 1.00 | 62.75 | C |
| ATOM | 7703 | C | ALA | C | 228 | −34.127 | −28.876 | 77.299 | 1.00 | 64.61 | C |
| ATOM | 7704 | O | ALA | C | 228 | −35.203 | −28.334 | 77.169 | 1.00 | 62.45 | O |
| ATOM | 7705 | CB | ALA | C | 228 | −32.368 | −28.481 | 75.577 | 1.00 | 65.82 | C |
| ATOM | 7706 | N | TRP | C | 229 | −34.001 | −30.156 | 77.656 | 1.00 | 64.04 | N |
| ATOM | 7707 | CA | TRP | C | 229 | −35.170 | −30.992 | 77.877 | 1.00 | 57.86 | C |
| ATOM | 7708 | CB | TRP | C | 229 | −34.784 | −32.455 | 78.155 | 1.00 | 53.89 | C |
| ATOM | 7709 | CG | TRP | C | 229 | −34.241 | −32.734 | 79.511 | 1.00 | 55.40 | C |
| ATOM | 7710 | CD1 | TRP | C | 229 | −32.931 | −32.777 | 79.872 | 1.00 | 54.68 | C |
| ATOM | 7711 | NE1 | TRP | C | 229 | −32.820 | −33.075 | 81.209 | 1.00 | 64.95 | N |
| ATOM | 7712 | CE2 | TRP | C | 229 | −34.075 | −33.241 | 81.735 | 1.00 | 54.65 | C |
| ATOM | 7713 | CD2 | TRP | C | 229 | −34.997 | −33.038 | 80.692 | 1.00 | 59.58 | C |
| ATOM | 7714 | CE3 | TRP | C | 229 | −36.364 | −33.149 | 80.971 | 1.00 | 52.21 | C |
| ATOM | 7715 | CZ3 | TRP | C | 229 | −36.756 | −33.454 | 82.263 | 1.00 | 45.89 | C |
| ATOM | 7716 | CH2 | TRP | C | 229 | −35.811 | −33.649 | 83.276 | 1.00 | 46.93 | C |
| ATOM | 7717 | CZ2 | TRP | C | 229 | −34.470 | −33.545 | 83.033 | 1.00 | 47.46 | C |
| ATOM | 7718 | C | TRP | C | 229 | −36.110 | −30.411 | 78.927 | 1.00 | 55.44 | C |
| ATOM | 7719 | O | TRP | C | 229 | −37.308 | −30.385 | 78.758 | 1.00 | 55.29 | O |
| ATOM | 7720 | N | MET | C | 230 | −35.548 | −29.952 | 80.022 | 1.00 | 59.92 | N |
| ATOM | 7721 | CA | MET | C | 230 | −36.320 | −29.375 | 81.091 | 1.00 | 55.66 | C |
| ATOM | 7722 | CB | MET | C | 230 | −35.397 | −28.945 | 82.234 | 1.00 | 56.95 | C |
| ATOM | 7723 | CG | MET | C | 230 | −34.280 | −29.926 | 82.534 | 1.00 | 65.94 | C |
| ATOM | 7724 | SD | MET | C | 230 | −33.169 | −29.344 | 83.826 | 1.00 | 82.63 | S |
| ATOM | 7725 | CE | MET | C | 230 | −34.281 | −29.391 | 85.230 | 1.00 | 88.83 | C |
| ATOM | 7726 | C | MET | C | 230 | −37.112 | −28.171 | 80.629 | 1.00 | 52.19 | C |
| ATOM | 7727 | O | MET | C | 230 | −38.272 | −28.024 | 80.927 | 1.00 | 49.65 | O |
| ATOM | 7728 | N | PHE | C | 231 | −36.466 | −27.285 | 79.902 | 1.00 | 56.00 | N |
| ATOM | 7729 | CA | PHE | C | 231 | −37.153 | −26.110 | 79.409 | 1.00 | 51.86 | C |
| ATOM | 7730 | CB | PHE | C | 231 | −36.186 | −25.110 | 78.775 | 1.00 | 51.71 | C |
| ATOM | 7731 | CG | PHE | C | 231 | −35.394 | −24.331 | 79.787 | 1.00 | 57.14 | C |
| ATOM | 7732 | CD1 | PHE | C | 231 | −35.985 | −23.299 | 80.500 | 1.00 | 58.65 | C |
| ATOM | 7733 | CE1 | PHE | C | 231 | −35.264 | −22.586 | 81.444 | 1.00 | 59.70 | C |
| ATOM | 7734 | CZ | PHE | C | 231 | −33.941 | −22.903 | 81.687 | 1.00 | 65.51 | C |
| ATOM | 7735 | CE2 | PHE | C | 231 | −33.342 | −23.934 | 80.986 | 1.00 | 63.79 | C |
| ATOM | 7736 | CD2 | PHE | C | 231 | −34.069 | −24.644 | 80.044 | 1.00 | 58.45 | C |
| ATOM | 7737 | C | PHE | C | 231 | −38.308 | −26.463 | 78.512 | 1.00 | 52.38 | C |
| ATOM | 7738 | O | PHE | C | 231 | −39.363 | −25.893 | 78.624 | 1.00 | 51.10 | O |
| ATOM | 7739 | N | ILE | C | 232 | −38.094 | −27.428 | 77.636 | 1.00 | 53.07 | N |
| ATOM | 7740 | CA | ILE | C | 232 | −39.125 | −27.900 | 76.730 | 1.00 | 52.45 | C |
| ATOM | 7741 | CB | ILE | C | 232 | −38.579 | −29.009 | 75.816 | 1.00 | 50.33 | C |
| ATOM | 7742 | CG1 | ILE | C | 232 | −37.396 | −28.488 | 74.997 | 1.00 | 48.92 | C |
| ATOM | 7743 | CD1 | ILE | C | 232 | −36.454 | −29.593 | 74.498 | 1.00 | 68.38 | C |
| ATOM | 7744 | CG2 | ILE | C | 232 | −39.669 | −29.533 | 74.898 | 1.00 | 43.20 | C |
| ATOM | 7745 | C | ILE | C | 232 | −40.348 | −28.442 | 77.490 | 1.00 | 48.57 | C |
| ATOM | 7746 | O | ILE | C | 232 | −41.466 | −28.104 | 77.175 | 1.00 | 47.08 | O |
| ATOM | 7747 | N | CYS | C | 233 | −40.130 | −29.220 | 78.550 | 1.00 | 44.91 | N |
| ATOM | 7748 | CA | CYS | C | 233 | −41.243 | −29.689 | 79.378 | 1.00 | 41.10 | C |
| ATOM | 7749 | CB | CYS | C | 233 | −40.746 | −30.685 | 80.419 | 1.00 | 33.38 | C |
| ATOM | 7750 | SG | CYS | C | 233 | −40.128 | −32.201 | 79.689 | 1.00 | 46.76 | S |
| ATOM | 7751 | C | CYS | C | 233 | −42.030 | −28.551 | 80.052 | 1.00 | 48.71 | C |
| ATOM | 7752 | O | CYS | C | 233 | −43.236 | −28.545 | 80.033 | 1.00 | 46.78 | O |
| ATOM | 7753 | N | ILE | C | 234 | −41.351 | −27.583 | 80.653 | 1.00 | 44.95 | N |
| ATOM | 7754 | CA | ILE | C | 234 | −42.042 | −26.489 | 81.307 | 1.00 | 41.85 | C |
| ATOM | 7755 | CB | ILE | C | 234 | −41.067 | −25.600 | 82.090 | 1.00 | 41.08 | C |
| ATOM | 7756 | CG1 | ILE | C | 234 | −40.322 | −26.435 | 83.131 | 1.00 | 45.68 | C |
| ATOM | 7757 | CD1 | ILE | C | 234 | −39.248 | −25.662 | 83.883 | 1.00 | 48.37 | C |
| ATOM | 7758 | CG2 | ILE | C | 234 | −41.807 | −24.453 | 82.764 | 1.00 | 37.77 | C |
| ATOM | 7759 | C | ILE | C | 234 | −42.803 | −25.660 | 80.310 | 1.00 | 42.73 | C |
| ATOM | 7760 | O | ILE | C | 234 | −43.925 | −25.293 | 80.538 | 1.00 | 41.42 | O |
| ATOM | 7761 | N | GLY | C | 235 | −42.171 | −25.395 | 79.189 | 1.00 | 43.76 | N |
| ATOM | 7762 | CA | GLY | C | 235 | −42.616 | −24.572 | 78.090 | 1.00 | 41.36 | C |
| ATOM | 7763 | C | GLY | C | 235 | −43.672 | −25.054 | 77.184 | 1.00 | 40.41 | C |
| ATOM | 7764 | O | GLY | C | 235 | −44.629 | −24.355 | 76.997 | 1.00 | 38.72 | O |
| ATOM | 7765 | N | TRP | C | 236 | −43.523 | −26.218 | 76.588 | 1.00 | 41.89 | N |
| ATOM | 7766 | CA | TRP | C | 236 | −44.544 | −26.791 | 75.733 | 1.00 | 36.45 | C |
| ATOM | 7767 | CB | TRP | C | 236 | −43.920 | −27.436 | 74.499 | 1.00 | 34.80 | C |
| ATOM | 7768 | CG | TRP | C | 236 | −43.215 | −26.421 | 73.658 | 1.00 | 41.14 | C |
| ATOM | 7769 | CD1 | TRP | C | 236 | −41.868 | −26.259 | 73.520 | 1.00 | 39.00 | C |
| ATOM | 7770 | NE1 | TRP | C | 236 | −41.605 | −25.201 | 72.685 | 1.00 | 38.46 | N |
| ATOM | 7771 | CE2 | TRP | C | 236 | −42.790 | −24.649 | 72.281 | 1.00 | 36.35 | C |
| ATOM | 7772 | CD2 | TRP | C | 236 | −43.827 | −25.388 | 72.880 | 1.00 | 33.90 | C |
| ATOM | 7773 | CE3 | TRP | C | 236 | −45.150 | −25.024 | 72.621 | 1.00 | 29.91 | C |
| ATOM | 7774 | CZ3 | TRP | C | 236 | −45.389 | −23.953 | 71.786 | 1.00 | 34.79 | C |
| ATOM | 7775 | CH2 | TRP | C | 236 | −44.336 | −23.238 | 71.209 | 1.00 | 43.50 | C |
| ATOM | 7776 | CZ2 | TRP | C | 236 | −43.033 | −23.572 | 71.444 | 1.00 | 45.85 | C |
| ATOM | 7777 | C | TRP | C | 236 | −45.312 | −27.820 | 76.577 | 1.00 | 38.45 | C |
| ATOM | 7778 | O | TRP | C | 236 | −46.490 | −28.010 | 76.428 | 1.00 | 39.67 | O |
| ATOM | 7779 | N | GLY | C | 237 | −44.605 | −28.591 | 77.369 | 1.00 | 40.80 | N |
| ATOM | 7780 | CA | GLY | C | 237 | −45.227 | −29.703 | 78.043 | 1.00 | 43.43 | C |
| ATOM | 7781 | C | GLY | C | 237 | −46.275 | −29.529 | 79.113 | 1.00 | 47.02 | C |
| ATOM | 7782 | O | GLY | C | 237 | −47.312 | −30.161 | 79.085 | 1.00 | 46.80 | O |
| ATOM | 7783 | N | VAL | C | 238 | −45.965 | −28.712 | 80.109 | 1.00 | 46.51 | N |
| ATOM | 7784 | CA | VAL | C | 238 | −46.805 | −28.516 | 81.271 | 1.00 | 42.33 | C |
| ATOM | 7785 | CB | VAL | C | 238 | −46.051 | −27.797 | 82.430 | 1.00 | 38.75 | C |
| ATOM | 7786 | CG1 | VAL | C | 238 | −46.989 | −27.444 | 83.576 | 1.00 | 30.53 | C |
| ATOM | 7787 | CG2 | VAL | C | 238 | −44.930 | −28.678 | 82.935 | 1.00 | 42.01 | C |
| ATOM | 7788 | C | VAL | C | 238 | −48.099 | −27.850 | 80.922 | 1.00 | 44.00 | C |
| ATOM | 7789 | O | VAL | C | 238 | −49.099 | −28.199 | 81.503 | 1.00 | 45.78 | O |
| ATOM | 7790 | N | PRO | C | 239 | −48.196 | −26.837 | 80.053 | 1.00 | 44.91 | N |
| ATOM | 7791 | CA | PRO | C | 239 | −49.454 | −26.171 | 79.837 | 1.00 | 48.10 | C |
| ATOM | 7792 | CB | PRO | C | 239 | −49.133 | −25.151 | 78.730 | 1.00 | 46.69 | C |
| ATOM | 7793 | CG | PRO | C | 239 | −47.629 | −25.137 | 78.577 | 1.00 | 41.12 | C |
| ATOM | 7794 | CD | PRO | C | 239 | −47.079 | −25.949 | 79.695 | 1.00 | 42.18 | C |
| ATOM | 7795 | C | PRO | C | 239 | −50.563 | −27.117 | 79.368 | 1.00 | 43.70 | C |
| ATOM | 7796 | O | PRO | C | 239 | −51.719 | −26.845 | 79.616 | 1.00 | 38.78 | O |
| ATOM | 7797 | N | PHE | C | 240 | −50.236 | −28.194 | 78.685 | 1.00 | 42.98 | N |
| ATOM | 7798 | CA | PHE | C | 240 | −51.247 | −29.128 | 78.270 | 1.00 | 44.50 | C |
| ATOM | 7799 | CB | PHE | C | 240 | −50.695 | −30.217 | 77.331 | 1.00 | 42.89 | C |
| ATOM | 7800 | CG | PHE | C | 240 | −51.759 | −31.106 | 76.744 | 1.00 | 42.68 | C |
| ATOM | 7801 | CD1 | PHE | C | 240 | −52.873 | −30.562 | 76.115 | 1.00 | 40.04 | C |
| ATOM | 7802 | CE1 | PHE | C | 240 | −53.859 | −31.383 | 75.579 | 1.00 | 40.48 | C |
| ATOM | 7803 | CZ | PHE | C | 240 | −53.731 | −32.759 | 75.668 | 1.00 | 43.37 | C |
| ATOM | 7804 | CE2 | PHE | C | 240 | −52.621 | −33.308 | 76.290 | 1.00 | 40.52 | C |
| ATOM | 7805 | CD2 | PHE | C | 240 | −51.646 | −32.484 | 76.823 | 1.00 | 37.72 | C |
| ATOM | 7806 | C | PHE | C | 240 | −52.085 | −29.698 | 79.457 | 1.00 | 45.99 | C |
| ATOM | 7807 | O | PHE | C | 240 | −53.276 | −29.517 | 79.439 | 1.00 | 43.16 | O |
| ATOM | 7808 | N | PRO | C | 241 | −51.584 | −30.257 | 80.582 | 1.00 | 44.20 | N |
| ATOM | 7809 | CA | PRO | C | 241 | −52.313 | −30.706 | 81.753 | 1.00 | 42.03 | C |
| ATOM | 7810 | CB | PRO | C | 241 | −51.198 | −31.082 | 82.726 | 1.00 | 41.85 | C |
| ATOM | 7811 | CG | PRO | C | 241 | −50.159 | −31.648 | 81.840 | 1.00 | 46.02 | C |
| ATOM | 7812 | CD | PRO | C | 241 | −50.210 | −30.765 | 80.612 | 1.00 | 46.10 | C |
| ATOM | 7813 | C | PRO | C | 241 | −53.211 | −29.635 | 82.328 | 1.00 | 44.04 | C |
| ATOM | 7814 | O | PRO | C | 241 | −54.303 | −29.938 | 82.738 | 1.00 | 47.44 | O |
| ATOM | 7815 | N | ILE | C | 242 | −52.734 | −28.411 | 82.388 | 1.00 | 41.44 | N |
| ATOM | 7816 | CA | ILE | C | 242 | −53.481 | −27.303 | 82.922 | 1.00 | 43.90 | C |
| ATOM | 7817 | CB | ILE | C | 242 | −52.628 | −26.025 | 82.915 | 1.00 | 44.81 | C |
| ATOM | 7818 | CG1 | ILE | C | 242 | −51.438 | −26.179 | 83.865 | 1.00 | 43.43 | C |
| ATOM | 7819 | CD1 | ILE | C | 242 | −50.462 | −25.025 | 83.816 | 1.00 | 37.22 | C |
| ATOM | 7820 | CG2 | ILE | C | 242 | −53.475 | −24.816 | 83.278 | 1.00 | 44.47 | C |
| ATOM | 7821 | C | ILE | C | 242 | −54.727 | −27.061 | 82.063 | 1.00 | 46.68 | C |
| ATOM | 7822 | O | ILE | C | 242 | −55.838 | −27.024 | 82.545 | 1.00 | 50.89 | O |
| ATOM | 7823 | N | ILE | C | 243 | −54.526 | −27.030 | 80.754 | 1.00 | 47.27 | N |
| ATOM | 7824 | CA | ILE | C | 243 | −55.603 | −26.931 | 79.772 | 1.00 | 41.37 | C |
| ATOM | 7825 | CB | ILE | C | 243 | −55.067 | −26.762 | 78.332 | 1.00 | 46.30 | C |
| ATOM | 7826 | CG1 | ILE | C | 243 | −54.364 | −25.416 | 78.193 | 1.00 | 47.10 | C |
| ATOM | 7827 | CD1 | ILE | C | 243 | −55.279 | −24.242 | 78.427 | 1.00 | 48.08 | C |
| ATOM | 7828 | CG2 | ILE | C | 243 | −56.197 | −26.821 | 77.318 | 1.00 | 37.85 | C |
| ATOM | 7829 | C | ILE | C | 243 | −56.575 | −28.086 | 79.831 | 1.00 | 43.87 | C |
| ATOM | 7830 | O | ILE | C | 243 | −57.759 | −27.908 | 79.785 | 1.00 | 49.32 | O |
| ATOM | 7831 | N | VAL | C | 244 | −56.073 | −29.295 | 79.984 | 1.00 | 48.38 | N |
| ATOM | 7832 | CA | VAL | C | 244 | −56.945 | −30.448 | 80.112 | 1.00 | 48.41 | C |
| ATOM | 7833 | CB | VAL | C | 244 | −56.138 | −31.750 | 80.308 | 1.00 | 46.85 | C |
| ATOM | 7834 | CG1 | VAL | C | 244 | −57.057 | −32.903 | 80.691 | 1.00 | 47.59 | C |
| ATOM | 7835 | CG2 | VAL | C | 244 | −55.351 | −32.083 | 79.049 | 1.00 | 42.91 | C |
| ATOM | 7836 | C | VAL | C | 244 | −57.875 | −30.254 | 81.296 | 1.00 | 50.00 | C |
| ATOM | 7837 | O | VAL | C | 244 | −59.066 | −30.343 | 81.157 | 1.00 | 52.70 | O |
| ATOM | 7838 | N | ALA | C | 245 | −57.309 | −29.880 | 82.436 | 1.00 | 44.94 | N |
| ATOM | 7839 | CA | ALA | C | 245 | −58.060 | −29.579 | 83.647 | 1.00 | 44.96 | C |
| ATOM | 7840 | CB | ALA | C | 245 | −57.109 | −29.280 | 84.798 | 1.00 | 40.35 | C |
| ATOM | 7841 | C | ALA | C | 245 | −59.046 | −28.431 | 83.460 | 1.00 | 45.27 | C |
| ATOM | 7842 | O | ALA | C | 245 | −60.153 | −28.455 | 83.949 | 1.00 | 49.76 | O |
| ATOM | 7843 | N | TRP | C | 246 | −58.675 | −27.450 | 82.683 | 1.00 | 43.88 | N |
| ATOM | 7844 | CA | TRP | C | 246 | −59.585 | −26.384 | 82.367 | 1.00 | 47.27 | C |
| ATOM | 7845 | CB | TRP | C | 246 | −58.831 | −25.299 | 81.590 | 1.00 | 43.82 | C |
| ATOM | 7846 | CG | TRP | C | 246 | −59.665 | −24.214 | 80.988 | 1.00 | 45.21 | C |
| ATOM | 7847 | CD1 | TRP | C | 246 | −60.222 | −23.158 | 81.638 | 1.00 | 52.06 | C |
| ATOM | 7848 | NE1 | TRP | C | 246 | −60.897 | −22.357 | 80.752 | 1.00 | 51.41 | N |
| ATOM | 7849 | CE2 | TRP | C | 246 | −60.773 | −22.884 | 79.495 | 1.00 | 49.51 | C |
| ATOM | 7850 | CD2 | TRP | C | 246 | −59.996 | −24.053 | 79.603 | 1.00 | 45.48 | C |
| ATOM | 7851 | CE3 | TRP | C | 246 | −59.721 | −24.787 | 78.447 | 1.00 | 45.57 | C |
| ATOM | 7852 | CZ3 | TRP | C | 246 | −60.222 | −24.335 | 77.240 | 1.00 | 47.51 | C |
| ATOM | 7853 | CH2 | TRP | C | 246 | −60.992 | −23.170 | 77.168 | 1.00 | 53.27 | C |
| ATOM | 7854 | CZ2 | TRP | C | 246 | −61.277 | −22.433 | 78.283 | 1.00 | 56.08 | C |
| ATOM | 7855 | C | TRP | C | 246 | −60.796 | −26.915 | 81.571 | 1.00 | 50.84 | C |
| ATOM | 7856 | O | TRP | C | 246 | −61.918 | −26.522 | 81.794 | 1.00 | 49.61 | O |
| ATOM | 7857 | N | ALA | C | 247 | −60.527 | −27.831 | 80.645 | 1.00 | 51.22 | N |
| ATOM | 7858 | CA | ALA | C | 247 | −61.523 | −28.473 | 79.778 | 1.00 | 57.94 | C |
| ATOM | 7859 | CB | ALA | C | 247 | −60.863 | −29.465 | 78.823 | 1.00 | 56.50 | C |
| ATOM | 7860 | C | ALA | C | 247 | −62.646 | −29.134 | 80.564 | 1.00 | 62.01 | C |
| ATOM | 7861 | O | ALA | C | 247 | −63.824 | −29.017 | 80.263 | 1.00 | 64.12 | O |
| ATOM | 7862 | N | ILE | C | 248 | −62.253 | −29.809 | 81.618 | 1.00 | 54.56 | N |
| ATOM | 7863 | CA | ILE | C | 248 | −63.173 | −30.497 | 82.458 | 1.00 | 57.62 | C |
| ATOM | 7864 | CB | ILE | C | 248 | −62.431 | −31.399 | 83.448 | 1.00 | 46.09 | C |
| ATOM | 7865 | CG1 | ILE | C | 248 | −61.519 | −32.348 | 82.670 | 1.00 | 48.39 | C |
| ATOM | 7866 | CD1 | ILE | C | 248 | −60.756 | −33.320 | 83.527 | 1.00 | 57.83 | C |
| ATOM | 7867 | CG2 | ILE | C | 248 | −63.414 | −32.175 | 84.307 | 1.00 | 49.36 | C |
| ATOM | 7868 | C | ILE | C | 248 | −64.088 | −29.487 | 83.188 | 1.00 | 66.16 | C |
| ATOM | 7869 | O | ILE | C | 248 | −65.298 | −29.668 | 83.287 | 1.00 | 74.05 | O |
| ATOM | 7870 | N | GLY | C | 249 | −63.501 | −28.374 | 83.639 | 1.00 | 59.39 | N |
| ATOM | 7871 | CA | GLY | C | 249 | −64.236 | −27.328 | 84.310 | 1.00 | 62.55 | C |
| ATOM | 7872 | C | GLY | C | 249 | −65.345 | −26.734 | 83.494 | 1.00 | 62.13 | C |
| ATOM | 7873 | O | GLY | C | 249 | −66.440 | −26.465 | 83.952 | 1.00 | 75.02 | O |
| ATOM | 7874 | N | LYS | C | 250 | −65.076 | −26.539 | 82.237 | 1.00 | 61.61 | N |
| ATOM | 7875 | CA | LYS | C | 250 | −66.081 | −26.045 | 81.332 | 1.00 | 68.04 | C |
| ATOM | 7876 | CB | LYS | C | 250 | −65.459 | −25.732 | 79.965 | 1.00 | 70.40 | C |
| ATOM | 7877 | CG | LYS | C | 250 | −64.168 | −24.920 | 80.034 | 1.00 | 67.67 | C |
| ATOM | 7878 | CD | LYS | C | 250 | −64.383 | −23.568 | 80.701 | 1.00 | 69.69 | C |
| ATOM | 7879 | CE | LYS | C | 250 | −65.164 | −22.619 | 79.805 | 1.00 | 67.79 | C |
| ATOM | 7880 | NZ | LYS | C | 250 | −65.325 | −21.274 | 80.427 | 1.00 | 72.10 | N |
| ATOM | 7881 | C | LYS | C | 250 | −67.206 | −27.039 | 81.179 | 1.00 | 66.13 | C |
| ATOM | 7882 | O | LYS | C | 250 | −68.367 | −26.689 | 81.176 | 1.00 | 70.53 | O |
| ATOM | 7883 | O | LEU | C | 251 | −70.071 | −29.437 | 81.895 | 1.00 | 81.49 | O |
| ATOM | 7884 | N | LEU | C | 251 | −66.856 | −28.308 | 81.058 | 1.00 | 65.01 | N |
| ATOM | 7885 | CA | LEU | C | 251 | −67.878 | −29.340 | 80.929 | 1.00 | 70.85 | C |
| ATOM | 7886 | C | LEU | C | 251 | −68.868 | −29.322 | 82.094 | 1.00 | 74.24 | C |
| ATOM | 7887 | CB | LEU | C | 251 | −67.260 | −30.730 | 80.791 | 1.00 | 69.83 | C |
| ATOM | 7888 | CG | LEU | C | 251 | −68.313 | −31.830 | 80.635 | 1.00 | 58.47 | C |
| ATOM | 7889 | CD1 | LEU | C | 251 | −69.212 | −31.543 | 79.442 | 1.00 | 51.08 | C |
| ATOM | 7890 | CD2 | LEU | C | 251 | −67.665 | −33.192 | 80.508 | 1.00 | 69.86 | C |
| ATOM | 7891 | N | TYR | C | 252 | −68.356 | −29.125 | 83.309 | 1.00 | 71.35 | N |
| ATOM | 7892 | CA | TYR | C | 252 | −69.236 | −29.087 | 84.466 | 1.00 | 76.24 | C |
| ATOM | 7893 | CB | TYR | C | 252 | −68.396 | −29.348 | 85.719 | 1.00 | 77.11 | C |
| ATOM | 7894 | CG | TYR | C | 252 | −68.050 | −30.776 | 86.066 | 1.00 | 84.54 | C |
| ATOM | 7895 | CD1 | TYR | C | 252 | −68.528 | −31.851 | 85.328 | 1.00 | 79.84 | C |
| ATOM | 7896 | CE1 | TYR | C | 252 | −68.202 | −33.152 | 85.683 | 1.00 | 77.35 | C |
| ATOM | 7897 | CZ | TYR | C | 252 | −67.399 | −33.379 | 86.786 | 1.00 | 81.64 | C |
| ATOM | 7898 | OH | TYR | C | 252 | −67.067 | −34.660 | 87.156 | 1.00 | 83.59 | O |
| ATOM | 7899 | CE2 | TYR | C | 252 | −66.922 | −32.327 | 87.531 | 1.00 | 86.24 | C |
| ATOM | 7900 | CD2 | TYR | C | 252 | −67.249 | −31.042 | 87.172 | 1.00 | 90.19 | C |
| ATOM | 7901 | C | TYR | C | 252 | −69.916 | −27.736 | 84.760 | 1.00 | 78.05 | C |
| ATOM | 7902 | O | TYR | C | 252 | −71.136 | −27.652 | 84.876 | 1.00 | 80.32 | O |
| ATOM | 7903 | N | TYR | C | 253 | −69.114 | −26.690 | 84.947 | 1.00 | 75.75 | N |
| ATOM | 7904 | CA | TYR | C | 253 | −69.643 | −25.391 | 85.339 | 1.00 | 69.59 | C |
| ATOM | 7905 | CB | TYR | C | 253 | −68.652 | −24.661 | 86.247 | 1.00 | 67.22 | C |
| ATOM | 7906 | CG | TYR | C | 253 | −68.189 | −25.527 | 87.402 | 1.00 | 79.18 | C |
| ATOM | 7907 | CD1 | TYR | C | 253 | −68.992 | −25.715 | 88.519 | 1.00 | 85.24 | C |
| ATOM | 7908 | CE1 | TYR | C | 253 | −68.578 | −26.510 | 89.574 | 1.00 | 95.28 | C |
| ATOM | 7909 | CZ | TYR | C | 253 | −67.347 | −27.132 | 89.521 | 1.00 | 94.44 | C |
| ATOM | 7910 | OH | TYR | C | 253 | −66.929 | −27.921 | 90.570 | 1.00 | 91.17 | O |
| ATOM | 7911 | CE2 | TYR | C | 253 | −66.530 | −26.966 | 88.422 | 1.00 | 91.21 | C |
| ATOM | 7912 | CD2 | TYR | C | 253 | −66.954 | −26.169 | 87.369 | 1.00 | 85.02 | C |
| ATOM | 7913 | C | TYR | C | 253 | −70.101 | −24.547 | 84.111 | 1.00 | 77.59 | C |
| ATOM | 7914 | O | TYR | C | 253 | −71.180 | −23.961 | 84.143 | 1.00 | 84.01 | O |
| ATOM | 7915 | N | ASP | C | 254 | −69.214 | −24.351 | 83.115 | 1.00 | 77.86 | N |
| ATOM | 7916 | CA | ASP | C | 254 | −69.496 | −23.373 | 82.009 | 1.00 | 79.29 | C |
| ATOM | 7917 | CB | ASP | C | 254 | −68.544 | −22.173 | 82.144 | 1.00 | 78.35 | C |
| ATOM | 7918 | CG | ASP | C | 254 | −69.051 | −20.921 | 81.441 | 1.00 | 93.53 | C |
| ATOM | 7919 | OD2 | ASP | C | 254 | −68.280 | −19.937 | 81.379 | 1.00 | 97.32 | O |
| ATOM | 7920 | OD1 | ASP | C | 254 | −70.211 | −20.908 | 80.972 | 1.00 | 95.54 | O |
| ATOM | 7921 | C | ASP | C | 254 | −69.382 | −24.006 | 80.599 | 1.00 | 81.44 | C |
| ATOM | 7922 | O | ASP | C | 254 | −68.289 | −24.286 | 80.150 | 1.00 | 83.85 | O |
| ATOM | 7923 | N | ASN | C | 255 | −70.499 | −24.271 | 79.914 | 1.00 | 81.91 | N |
| ATOM | 7924 | CA | ASN | C | 255 | −70.453 | −24.881 | 78.577 | 1.00 | 76.81 | C |
| ATOM | 7925 | CB | ASN | C | 255 | −71.260 | −26.179 | 78.535 | 1.00 | 83.65 | C |
| ATOM | 7926 | CG | ASN | C | 255 | −70.955 | −27.012 | 77.305 | 1.00 | 85.23 | C |
| ATOM | 7927 | OD1 | ASN | C | 255 | −69.874 | −27.590 | 77.186 | 1.00 | 75.77 | O |
| ATOM | 7928 | ND2 | ASN | C | 255 | −71.909 | −27.077 | 76.381 | 1.00 | 81.54 | N |
| ATOM | 7929 | C | ASN | C | 255 | −70.928 | −23.913 | 77.501 | 1.00 | 82.33 | C |
| ATOM | 7930 | O | ASN | C | 255 | −71.171 | −24.269 | 76.360 | 1.00 | 77.05 | O |
| ATOM | 7931 | N | GLU | C | 256 | −71.089 | −22.676 | 77.894 | 1.00 | 92.20 | N |
| ATOM | 7932 | CA | GLU | C | 256 | −71.577 | −21.642 | 77.020 | 1.00 | 94.80 | C |
| ATOM | 7933 | CB | GLU | C | 256 | −71.982 | −20.419 | 77.852 | 1.00 | 89.65 | C |
| ATOM | 7934 | CG | GLU | C | 256 | −72.912 | −19.435 | 77.161 | 1.00 | 95.33 | C |
| ATOM | 7935 | CD | GLU | C | 256 | −74.373 | −19.810 | 77.311 | 1.00 | 99.69 | C |
| ATOM | 7936 | OE1 | GLU | C | 256 | −74.743 | −20.945 | 76.942 | 1.00 | 95.71 | O |
| ATOM | 7937 | OE2 | GLU | C | 256 | −75.152 | −18.967 | 77.806 | 1.00 | 97.16 | O |
| ATOM | 7938 | C | GLU | C | 256 | −70.500 | −21.236 | 75.965 | 1.00 | 98.90 | C |
| ATOM | 7939 | O | GLU | C | 256 | −69.294 | −21.344 | 76.218 | 1.00 | 88.68 | O |
| ATOM | 7940 | N | LYS | C | 257 | −71.009 | −20.664 | 74.837 | 1.00 | 104.06 | N |
| ATOM | 7941 | CA | LYS | C | 257 | −70.237 | −19.918 | 73.818 | 1.00 | 96.90 | C |
| ATOM | 7942 | CB | LYS | C | 257 | −70.257 | −18.413 | 74.124 | 1.00 | 100.20 | C |
| ATOM | 7943 | CG | LYS | C | 257 | −71.595 | −17.740 | 73.832 | 1.00 | 106.12 | C |
| ATOM | 7944 | CD | LYS | C | 257 | −71.592 | −16.262 | 74.208 | 1.00 | 108.27 | C |
| ATOM | 7945 | CE | LYS | C | 257 | −71.827 | −16.057 | 75.698 | 1.00 | 100.88 | C |
| ATOM | 7946 | NZ | LYS | C | 257 | −71.836 | −14.609 | 76.058 | 1.00 | 95.18 | N |
| ATOM | 7947 | C | LYS | C | 257 | −68.792 | −20.391 | 73.528 | 1.00 | 95.33 | C |
| ATOM | 7948 | O | LYS | C | 257 | −67.833 | −19.696 | 73.851 | 1.00 | 98.10 | O |
| ATOM | 7949 | N | CYS | C | 258 | −68.665 | −21.614 | 72.978 | 1.00 | 93.35 | N |
| ATOM | 7950 | CA | CYS | C | 258 | −67.376 | −22.260 | 72.609 | 1.00 | 79.72 | C |
| ATOM | 7951 | CB | CYS | C | 258 | −66.899 | −21.709 | 71.267 | 1.00 | 70.50 | C |
| ATOM | 7952 | SG | CYS | C | 258 | −68.128 | −21.826 | 69.964 | 1.00 | 84.01 | S |
| ATOM | 7953 | C | CYS | C | 258 | −66.262 | −22.125 | 73.615 | 1.00 | 78.14 | C |
| ATOM | 7954 | O | CYS | C | 258 | −65.114 | −21.950 | 73.240 | 1.00 | 77.41 | O |
| ATOM | 7955 | N | TRP | C | 259 | −66.599 | −22.202 | 74.892 | 1.00 | 82.58 | N |
| ATOM | 7956 | CA | TRP | C | 259 | −65.592 | −22.131 | 75.938 | 1.00 | 76.30 | C |
| ATOM | 7957 | CB | TRP | C | 259 | −64.537 | −23.245 | 75.802 | 1.00 | 68.95 | C |
| ATOM | 7958 | CG | TRP | C | 259 | −65.065 | −24.635 | 76.068 | 1.00 | 72.81 | C |
| ATOM | 7959 | CD1 | TRP | C | 259 | −66.362 | −24.993 | 76.292 | 1.00 | 74.09 | C |
| ATOM | 7960 | NE1 | TRP | C | 259 | −66.451 | −26.348 | 76.505 | 1.00 | 70.95 | N |
| ATOM | 7961 | CE2 | TRP | C | 259 | −65.197 | −26.893 | 76.424 | 1.00 | 68.99 | C |
| ATOM | 7962 | CD2 | TRP | C | 259 | −64.298 | −25.843 | 76.151 | 1.00 | 70.49 | C |
| ATOM | 7963 | CE3 | TRP | C | 259 | −62.939 | −26.140 | 76.018 | 1.00 | 68.72 | C |
| ATOM | 7964 | CZ3 | TRP | C | 259 | −62.528 | −27.453 | 76.162 | 1.00 | 65.35 | C |
| ATOM | 7965 | CH2 | TRP | C | 259 | −63.447 | −28.473 | 76.434 | 1.00 | 61.81 | C |
| ATOM | 7966 | CZ2 | TRP | C | 259 | −64.782 | −28.213 | 76.567 | 1.00 | 68.24 | C |
| ATOM | 7967 | C | TRP | C | 259 | −64.947 | −20.730 | 76.076 | 1.00 | 78.23 | C |
| ATOM | 7968 | O | TRP | C | 259 | −64.277 | −20.429 | 77.054 | 1.00 | 78.33 | O |
| ATOM | 7969 | N | ALA | C | 260 | −65.208 | −19.866 | 75.091 | 1.00 | 86.36 | N |
| ATOM | 7970 | CA | ALA | C | 260 | −64.695 | −18.509 | 75.070 | 1.00 | 88.34 | C |
| ATOM | 7971 | CB | ALA | C | 260 | −64.549 | −18.018 | 73.638 | 1.00 | 87.37 | C |
| ATOM | 7972 | C | ALA | C | 260 | −65.590 | −17.563 | 75.870 | 1.00 | 91.34 | C |
| ATOM | 7973 | O | ALA | C | 260 | −65.207 | −16.440 | 76.178 | 1.00 | 96.95 | O |
| ATOM | 7974 | N | GLY | C | 261 | −66.821 | −18.001 | 76.138 | 1.00 | 93.52 | N |
| ATOM | 7975 | CA | GLY | C | 261 | −67.784 | −17.146 | 76.781 | 1.00 | 97.10 | C |
| ATOM | 7976 | C | GLY | C | 261 | −67.674 | −17.066 | 78.289 | 1.00 | 101.10 | C |
| ATOM | 7977 | O | GLY | C | 261 | −67.593 | −18.086 | 78.977 | 1.00 | 102.04 | O |
| ATOM | 7978 | N | LYS | C | 262 | −67.734 | −15.807 | 78.781 | 1.00 | 97.65 | N |
| ATOM | 7979 | CA | LYS | C | 262 | −67.685 | −15.508 | 80.203 | 1.00 | 99.50 | C |
| ATOM | 7980 | C | LYS | C | 262 | −69.097 | −15.267 | 80.741 | 1.00 | 107.39 | C |
| ATOM | 7981 | O | LYS | C | 262 | −70.042 | −15.145 | 79.976 | 1.00 | 108.06 | O |
| ATOM | 7982 | CB | LYS | C | 262 | −66.790 | −14.300 | 80.481 | 1.00 | 99.35 | C |
| ATOM | 7983 | CG | LYS | C | 262 | −65.303 | −14.580 | 80.322 | 1.00 | 102.34 | C |
| ATOM | 7984 | CD | LYS | C | 262 | −64.472 | −13.341 | 80.620 | 1.00 | 116.59 | C |
| ATOM | 7985 | CE | LYS | C | 262 | −62.982 | −13.613 | 80.459 | 1.00 | 118.07 | C |
| ATOM | 7986 | NZ | LYS | C | 262 | −62.164 | −12.380 | 80.644 | 1.00 | 120.41 | N |
| ATOM | 7987 | O | ARG | C | 263 | −69.601 | −15.845 | 84.852 | 1.00 | 121.03 | O |
| ATOM | 7988 | N | ARG | C | 263 | −69.237 | −15.494 | 82.049 | 1.00 | 115.73 | N |
| ATOM | 7989 | CA | ARG | C | 263 | −70.533 | −15.552 | 82.719 | 1.00 | 114.43 | C |
| ATOM | 7990 | C | ARG | C | 263 | −70.349 | −15.136 | 84.187 | 1.00 | 123.48 | C |
| ATOM | 7991 | CB | ARG | C | 263 | −71.005 | −17.002 | 82.757 | 1.00 | 105.99 | C |
| ATOM | 7992 | CG | ARG | C | 263 | −72.022 | −17.443 | 81.733 | 1.00 | 95.47 | C |
| ATOM | 7993 | CD | ARG | C | 263 | −72.643 | −18.731 | 82.253 | 1.00 | 92.91 | C |
| ATOM | 7994 | NE | ARG | C | 263 | −73.474 | −19.432 | 81.283 | 1.00 | 96.07 | N |
| ATOM | 7995 | CZ | ARG | C | 263 | −74.272 | −20.448 | 81.599 | 1.00 | 99.27 | C |
| ATOM | 7996 | NH1 | ARG | C | 263 | −74.349 | −20.862 | 82.857 | 1.00 | 97.95 | N |
| ATOM | 7997 | NH2 | ARG | C | 263 | −74.998 | −21.045 | 80.664 | 1.00 | 106.64 | N |
| ATOM | 7998 | O | PRO | C | 264 | −72.980 | −13.478 | 87.553 | 1.00 | 127.37 | O |
| ATOM | 7999 | N | PRO | C | 264 | −70.975 | −14.000 | 84.718 | 1.00 | 126.77 | N |
| ATOM | 8000 | CA | PRO | C | 264 | −71.042 | −13.439 | 86.119 | 1.00 | 131.17 | C |
| ATOM | 8001 | C | PRO | C | 264 | −71.995 | −14.134 | 87.183 | 1.00 | 128.58 | C |
| ATOM | 8002 | CB | PRO | C | 264 | −71.497 | −12.001 | 85.877 | 1.00 | 127.00 | C |
| ATOM | 8003 | CG | PRO | C | 264 | −72.339 | −12.098 | 84.648 | 1.00 | 116.36 | C |
| ATOM | 8004 | CD | PRO | C | 264 | −71.687 | −13.131 | 83.766 | 1.00 | 114.64 | C |
| ATOM | 8005 | O | GLY | C | 265 | −70.192 | −16.815 | 89.582 | 1.00 | 113.14 | O |
| ATOM | 8006 | N | GLY | C | 265 | −71.880 | −15.398 | 87.623 | 1.00 | 123.97 | N |
| ATOM | 8007 | CA | GLY | C | 265 | −70.877 | −16.326 | 87.292 | 1.00 | 122.87 | C |
| ATOM | 8008 | C | GLY | C | 265 | −70.019 | −16.898 | 88.365 | 1.00 | 117.41 | C |
| ATOM | 8009 | O | VAL | C | 266 | −67.279 | −18.786 | 86.264 | 1.00 | 92.99 | O |
| ATOM | 8010 | N | VAL | C | 266 | −69.109 | −17.652 | 87.765 | 1.00 | 118.66 | N |
| ATOM | 8011 | CA | VAL | C | 266 | −68.182 | −18.536 | 88.446 | 1.00 | 117.54 | C |
| ATOM | 8012 | C | VAL | C | 266 | −67.043 | −18.707 | 87.467 | 1.00 | 101.69 | C |
| ATOM | 8013 | CB | VAL | C | 266 | −68.801 | −19.931 | 88.733 | 1.00 | 114.33 | C |
| ATOM | 8014 | CG1 | VAL | C | 266 | −67.710 | −20.981 | 88.966 | 1.00 | 91.04 | C |
| ATOM | 8015 | CG2 | VAL | C | 266 | −69.754 | −19.878 | 89.922 | 1.00 | 106.51 | C |
| ATOM | 8016 | O | TYR | C | 267 | −63.273 | −20.190 | 88.016 | 1.00 | 74.06 | O |
| ATOM | 8017 | N | TYR | C | 267 | −65.808 | −18.654 | 87.969 | 1.00 | 98.12 | N |
| ATOM | 8018 | CA | TYR | C | 267 | −64.710 | −18.473 | 87.079 | 1.00 | 93.58 | C |
| ATOM | 8019 | C | TYR | C | 267 | −63.903 | −19.756 | 87.056 | 1.00 | 83.96 | C |
| ATOM | 8020 | CB | TYR | C | 267 | −63.895 | −17.242 | 87.484 | 1.00 | 95.12 | C |
| ATOM | 8021 | CG | TYR | C | 267 | −64.751 | −15.986 | 87.527 | 1.00 | 111.29 | C |
| ATOM | 8022 | CD2 | TYR | C | 267 | −65.332 | −15.548 | 88.717 | 1.00 | 113.98 | C |
| ATOM | 8023 | CD1 | TYR | C | 267 | −65.006 | −15.256 | 86.371 | 1.00 | 119.11 | C |
| ATOM | 8024 | CE2 | TYR | C | 267 | −66.134 | −14.409 | 88.753 | 1.00 | 121.23 | C |
| ATOM | 8025 | CE1 | TYR | C | 267 | −65.795 | −14.111 | 86.399 | 1.00 | 127.44 | C |
| ATOM | 8026 | CZ | TYR | C | 267 | −66.351 | −13.694 | 87.589 | 1.00 | 129.84 | C |
| ATOM | 8027 | OH | TYR | C | 267 | −67.133 | −12.565 | 87.613 | 1.00 | 127.58 | O |
| ATOM | 8028 | N | THR | C | 268 | −63.971 | −20.331 | 85.868 | 1.00 | 77.99 | N |
| ATOM | 8029 | CA | THR | C | 268 | −63.321 | −21.537 | 85.512 | 1.00 | 68.91 | C |
| ATOM | 8030 | CB | THR | C | 268 | −64.221 | −22.426 | 84.621 | 1.00 | 70.50 | C |
| ATOM | 8031 | OG1 | THR | C | 268 | −64.717 | −21.661 | 83.514 | 1.00 | 69.19 | O |
| ATOM | 8032 | CG2 | THR | C | 268 | −65.396 | −22.963 | 85.417 | 1.00 | 77.38 | C |
| ATOM | 8033 | C | THR | C | 268 | −62.091 | −21.191 | 84.759 | 1.00 | 64.23 | C |
| ATOM | 8034 | O | THR | C | 268 | −61.179 | −21.974 | 84.664 | 1.00 | 63.79 | O |
| ATOM | 8035 | N | ASP | C | 269 | −62.071 | −20.001 | 84.180 | 1.00 | 59.48 | N |
| ATOM | 8036 | CA | ASP | C | 269 | −60.946 | −19.623 | 83.387 | 1.00 | 54.13 | C |
| ATOM | 8037 | CB | ASP | C | 269 | −61.289 | −18.420 | 82.507 | 1.00 | 52.76 | C |
| ATOM | 8038 | CG | ASP | C | 269 | −62.162 | −18.787 | 81.330 | 1.00 | 60.81 | C |
| ATOM | 8039 | OD1 | ASP | C | 269 | −62.956 | −19.743 | 81.450 | 1.00 | 66.00 | O |
| ATOM | 8040 | OD2 | ASP | C | 269 | −62.051 | −18.119 | 80.280 | 1.00 | 67.69 | O |
| ATOM | 8041 | C | ASP | C | 269 | −59.775 | −19.255 | 84.290 | 1.00 | 59.87 | C |
| ATOM | 8042 | O | ASP | C | 269 | −58.713 | −18.884 | 83.818 | 1.00 | 63.21 | O |
| ATOM | 8043 | N | TYR | C | 270 | −59.965 | −19.386 | 85.605 | 1.00 | 59.55 | N |
| ATOM | 8044 | CA | TYR | C | 270 | −58.878 | −19.268 | 86.553 | 1.00 | 59.58 | C |
| ATOM | 8045 | CB | TYR | C | 270 | −59.399 | −18.889 | 87.947 | 1.00 | 59.09 | C |
| ATOM | 8046 | CG | TYR | C | 270 | −59.727 | −17.415 | 88.084 | 1.00 | 67.18 | C |
| ATOM | 8047 | CD2 | TYR | C | 270 | −59.181 | −16.645 | 89.108 | 1.00 | 59.95 | C |
| ATOM | 8048 | CE2 | TYR | C | 270 | −59.484 | −15.300 | 89.226 | 1.00 | 61.83 | C |
| ATOM | 8049 | CZ | TYR | C | 270 | −60.342 | −14.715 | 88.311 | 1.00 | 75.41 | C |
| ATOM | 8050 | OH | TYR | C | 270 | −60.666 | −13.380 | 88.400 | 1.00 | 88.73 | O |
| ATOM | 8051 | CE1 | TYR | C | 270 | −60.888 | −15.459 | 87.293 | 1.00 | 70.96 | C |
| ATOM | 8052 | CD1 | TYR | C | 270 | −60.581 | −16.794 | 87.184 | 1.00 | 70.21 | C |
| ATOM | 8053 | C | TYR | C | 270 | −58.048 | −20.524 | 86.591 | 1.00 | 59.56 | C |
| ATOM | 8054 | O | TYR | C | 270 | −56.932 | −20.520 | 87.040 | 1.00 | 58.67 | O |
| ATOM | 8055 | N | ILE | C | 271 | −58.583 | −21.612 | 86.083 | 1.00 | 56.33 | N |
| ATOM | 8056 | CA | ILE | C | 271 | −57.835 | −22.852 | 86.025 | 1.00 | 58.86 | C |
| ATOM | 8057 | CB | ILE | C | 271 | −58.705 | −24.015 | 85.498 | 1.00 | 54.01 | C |
| ATOM | 8058 | CG1 | ILE | C | 271 | −59.925 | −24.205 | 86.401 | 1.00 | 52.25 | C |
| ATOM | 8059 | CD1 | ILE | C | 271 | −60.925 | −25.213 | 85.881 | 1.00 | 50.41 | C |
| ATOM | 8060 | CG2 | ILE | C | 271 | −57.904 | −25.303 | 85.445 | 1.00 | 46.14 | C |
| ATOM | 8061 | C | ILE | C | 271 | −56.536 | −22.738 | 85.222 | 1.00 | 52.07 | C |
| ATOM | 8062 | O | ILE | C | 271 | −55.501 | −23.191 | 85.676 | 1.00 | 47.55 | O |
| ATOM | 8063 | N | TYR | C | 272 | −56.583 | −22.058 | 84.065 | 1.00 | 50.74 | N |
| ATOM | 8064 | CA | TYR | C | 272 | −55.364 | −21.792 | 83.309 | 1.00 | 56.32 | C |
| ATOM | 8065 | CB | TYR | C | 272 | −55.560 | −21.976 | 81.798 | 1.00 | 50.66 | C |
| ATOM | 8066 | CG | TYR | C | 272 | −56.299 | −20.867 | 81.093 | 1.00 | 51.66 | C |
| ATOM | 8067 | CD2 | TYR | C | 272 | −55.614 | −19.795 | 80.530 | 1.00 | 52.49 | C |
| ATOM | 8068 | CE2 | TYR | C | 272 | −56.288 | −18.789 | 79.868 | 1.00 | 54.59 | C |
| ATOM | 8069 | CZ | TYR | C | 272 | −57.660 | −18.857 | 79.756 | 1.00 | 56.50 | C |
| ATOM | 8070 | OH | TYR | C | 272 | −58.345 | −17.862 | 79.098 | 1.00 | 67.68 | O |
| ATOM | 8071 | CE1 | TYR | C | 272 | −58.356 | −19.915 | 80.298 | 1.00 | 48.72 | C |
| ATOM | 8072 | CD1 | TYR | C | 272 | −57.677 | −20.911 | 80.955 | 1.00 | 49.08 | C |
| ATOM | 8073 | C | TYR | C | 272 | −54.696 | −20.440 | 83.650 | 1.00 | 60.79 | C |
| ATOM | 8074 | O | TYR | C | 272 | −53.483 | −20.339 | 83.803 | 1.00 | 61.44 | O |
| ATOM | 8075 | N | GLN | C | 273 | −55.521 | −19.406 | 83.829 | 1.00 | 60.17 | N |
| ATOM | 8076 | CA | GLN | C | 273 | −55.059 | −18.083 | 84.202 | 1.00 | 56.88 | C |
| ATOM | 8077 | CB | GLN | C | 273 | −56.218 | −17.087 | 84.265 | 1.00 | 51.61 | C |
| ATOM | 8078 | CG | GLN | C | 273 | −56.736 | −16.650 | 82.900 | 1.00 | 56.84 | C |
| ATOM | 8079 | CD | GLN | C | 273 | −58.016 | −15.851 | 83.012 | 1.00 | 61.26 | C |
| ATOM | 8080 | OE1 | GLN | C | 273 | −58.479 | −15.579 | 84.116 | 1.00 | 67.29 | O |
| ATOM | 8081 | NE2 | GLN | C | 273 | −58.598 | −15.474 | 81.875 | 1.00 | 60.23 | N |
| ATOM | 8082 | C | GLN | C | 273 | −54.306 | −18.076 | 85.513 | 1.00 | 58.34 | C |
| ATOM | 8083 | O | GLN | C | 273 | −53.358 | −17.345 | 85.687 | 1.00 | 61.00 | O |
| ATOM | 8084 | N | GLY | C | 274 | −54.763 | −18.879 | 86.451 | 1.00 | 55.19 | N |
| ATOM | 8085 | CA | GLY | C | 274 | −54.125 | −19.083 | 87.726 | 1.00 | 56.88 | C |
| ATOM | 8086 | C | GLY | C | 274 | −52.664 | −19.358 | 87.658 | 1.00 | 56.92 | C |
| ATOM | 8087 | O | GLY | C | 274 | −51.906 | −18.612 | 88.245 | 1.00 | 54.16 | O |
| ATOM | 8088 | N | PRO | C | 275 | −52.211 | −20.396 | 86.931 | 1.00 | 56.65 | N |
| ATOM | 8089 | CA | PRO | C | 275 | −50.821 | −20.737 | 86.736 | 1.00 | 56.89 | C |
| ATOM | 8090 | CB | PRO | C | 275 | −50.889 | −21.954 | 85.809 | 1.00 | 55.78 | C |
| ATOM | 8091 | CG | PRO | C | 275 | −52.141 | −22.631 | 86.189 | 1.00 | 48.23 | C |
| ATOM | 8092 | CD | PRO | C | 275 | −53.082 | −21.498 | 86.535 | 1.00 | 54.36 | C |
| ATOM | 8093 | C | PRO | C | 275 | −50.072 | −19.609 | 86.057 | 1.00 | 52.37 | C |
| ATOM | 8094 | O | PRO | C | 275 | −48.929 | −19.378 | 86.356 | 1.00 | 54.07 | O |
| ATOM | 8095 | N | MET | C | 276 | −50.699 | −18.919 | 85.142 | 1.00 | 51.19 | N |
| ATOM | 8096 | CA | MET | C | 276 | −50.048 | −17.839 | 84.458 | 1.00 | 54.53 | C |
| ATOM | 8097 | CB | MET | C | 276 | −50.942 | −17.294 | 83.347 | 1.00 | 46.82 | C |
| ATOM | 8098 | CG | MET | C | 276 | −51.127 | −18.292 | 82.229 | 1.00 | 45.75 | C |
| ATOM | 8099 | SD | MET | C | 276 | −52.555 | −17.947 | 81.202 | 1.00 | 55.45 | S |
| ATOM | 8100 | CE | MET | C | 276 | −52.010 | −16.478 | 80.341 | 1.00 | 46.05 | C |
| ATOM | 8101 | C | MET | C | 276 | −49.619 | −16.741 | 85.437 | 1.00 | 63.18 | C |
| ATOM | 8102 | O | MET | C | 276 | −48.486 | −16.295 | 85.445 | 1.00 | 51.52 | O |
| ATOM | 8103 | N | ALA | C | 277 | −50.520 | −16.403 | 86.359 | 1.00 | 69.24 | N |
| ATOM | 8104 | CA | ALA | C | 277 | −50.233 | −15.439 | 87.412 | 1.00 | 53.95 | C |
| ATOM | 8105 | CB | ALA | C | 277 | −51.508 | −15.078 | 88.160 | 1.00 | 42.32 | C |
| ATOM | 8106 | C | ALA | C | 277 | −49.176 | −15.966 | 88.377 | 1.00 | 52.05 | C |
| ATOM | 8107 | O | ALA | C | 277 | −48.284 | −15.246 | 88.748 | 1.00 | 55.08 | O |
| ATOM | 8108 | N | LEU | C | 278 | −49.256 | −17.246 | 88.743 | 1.00 | 53.78 | N |
| ATOM | 8109 | CA | LEU | C | 278 | −48.262 | −17.862 | 89.623 | 1.00 | 55.98 | C |
| ATOM | 8110 | CB | LEU | C | 278 | −48.647 | −19.309 | 89.954 | 1.00 | 55.98 | C |
| ATOM | 8111 | CG | LEU | C | 278 | −47.526 | −20.167 | 90.563 | 1.00 | 51.24 | C |
| ATOM | 8112 | CD1 | LEU | C | 278 | −47.322 | −19.844 | 92.035 | 1.00 | 47.07 | C |
| ATOM | 8113 | CD2 | LEU | C | 278 | −47.773 | −21.659 | 90.364 | 1.00 | 48.61 | C |
| ATOM | 8114 | C | LEU | C | 278 | −46.875 | −17.849 | 89.003 | 1.00 | 57.84 | C |
| ATOM | 8115 | O | LEU | C | 278 | −45.901 | −17.551 | 89.655 | 1.00 | 58.67 | O |
| ATOM | 8116 | N | VAL | C | 279 | −46.802 | −18.166 | 87.726 | 1.00 | 59.00 | N |
| ATOM | 8117 | CA | VAL | C | 279 | −45.564 | −18.150 | 86.977 | 1.00 | 57.92 | C |
| ATOM | 8118 | CB | VAL | C | 279 | −45.782 | −18.731 | 85.570 | 1.00 | 51.83 | C |
| ATOM | 8119 | CG1 | VAL | C | 279 | −44.843 | −18.097 | 84.561 | 1.00 | 58.17 | C |
| ATOM | 8120 | CG2 | VAL | C | 279 | −45.625 | −20.243 | 85.605 | 1.00 | 49.45 | C |
| ATOM | 8121 | C | VAL | C | 279 | −44.964 | −16.740 | 86.903 | 1.00 | 61.47 | C |
| ATOM | 8122 | O | VAL | C | 279 | −43.802 | −16.532 | 87.181 | 1.00 | 57.73 | O |
| ATOM | 8123 | N | LEU | C | 280 | −45.805 | −15.758 | 86.599 | 1.00 | 60.06 | N |
| ATOM | 8124 | CA | LEU | C | 280 | −45.406 | −14.357 | 86.587 | 1.00 | 57.60 | C |
| ATOM | 8125 | CB | LEU | C | 280 | −46.595 | −13.478 | 86.181 | 1.00 | 55.91 | C |
| ATOM | 8126 | CG | LEU | C | 280 | −46.396 | −11.962 | 86.140 | 1.00 | 48.51 | C |
| ATOM | 8127 | CD1 | LEU | C | 280 | −45.190 | −11.606 | 85.292 | 1.00 | 55.68 | C |
| ATOM | 8128 | CD2 | LEU | C | 280 | −47.641 | −11.278 | 85.600 | 1.00 | 37.59 | C |
| ATOM | 8129 | C | LEU | C | 280 | −44.867 | −13.914 | 87.926 | 1.00 | 57.74 | C |
| ATOM | 8130 | O | LEU | C | 280 | −43.877 | −13.210 | 88.003 | 1.00 | 55.36 | O |
| ATOM | 8131 | N | LEU | C | 281 | −45.562 | −14.347 | 88.979 | 1.00 | 55.00 | N |
| ATOM | 8132 | CA | LEU | C | 281 | −45.233 | −14.030 | 90.350 | 1.00 | 56.06 | C |
| ATOM | 8133 | CB | LEU | C | 281 | −46.221 | −14.689 | 91.315 | 1.00 | 57.32 | C |
| ATOM | 8134 | CG | LEU | C | 281 | −45.987 | −14.390 | 92.800 | 1.00 | 60.84 | C |
| ATOM | 8135 | CD1 | LEU | C | 281 | −46.146 | −12.906 | 93.085 | 1.00 | 50.87 | C |
| ATOM | 8136 | CD2 | LEU | C | 281 | −46.923 | −15.206 | 93.678 | 1.00 | 63.33 | C |
| ATOM | 8137 | C | LEU | C | 281 | −43.820 | −14.441 | 90.703 | 1.00 | 59.75 | C |
| ATOM | 8138 | O | LEU | C | 281 | −43.025 | −13.640 | 91.148 | 1.00 | 62.63 | O |
| ATOM | 8139 | N | ILE | C | 282 | −43.516 | −15.714 | 90.519 | 1.00 | 57.25 | N |
| ATOM | 8140 | CA | ILE | C | 282 | −42.198 | −16.238 | 90.815 | 1.00 | 56.47 | C |
| ATOM | 8141 | CB | ILE | C | 282 | −42.141 | −17.752 | 90.543 | 1.00 | 52.52 | C |
| ATOM | 8142 | CG1 | ILE | C | 282 | −43.194 | −18.476 | 91.385 | 1.00 | 54.03 | C |
| ATOM | 8143 | CD1 | ILE | C | 282 | −43.429 | −19.915 | 90.974 | 1.00 | 49.00 | C |
| ATOM | 8144 | CG2 | ILE | C | 282 | −40.750 | −18.304 | 90.814 | 1.00 | 44.48 | C |
| ATOM | 8145 | C | ILE | C | 282 | −41.150 | −15.540 | 89.969 | 1.00 | 57.10 | C |
| ATOM | 8146 | O | ILE | C | 282 | −40.032 | −15.302 | 90.389 | 1.00 | 60.05 | O |
| ATOM | 8147 | N | ASN | C | 283 | −41.549 | −15.138 | 88.776 | 1.00 | 58.25 | N |
| ATOM | 8148 | CA | ASN | C | 283 | −40.649 | −14.462 | 87.868 | 1.00 | 63.30 | C |
| ATOM | 8149 | CB | ASN | C | 283 | −41.256 | −14.351 | 86.468 | 1.00 | 64.12 | C |
| ATOM | 8150 | CG | ASN | C | 283 | −40.240 | −14.628 | 85.373 | 1.00 | 61.70 | C |
| ATOM | 8151 | OD1 | ASN | C | 283 | −39.232 | −15.303 | 85.598 | 1.00 | 57.64 | O |
| ATOM | 8152 | ND2 | ASN | C | 283 | −40.503 | −14.109 | 84.179 | 1.00 | 59.15 | N |
| ATOM | 8153 | C | ASN | C | 283 | −40.245 | −13.092 | 88.401 | 1.00 | 63.44 | C |
| ATOM | 8154 | O | ASN | C | 283 | −39.111 | −12.684 | 88.279 | 1.00 | 63.05 | O |
| ATOM | 8155 | N | PHE | C | 284 | −41.211 | −12.376 | 88.984 | 1.00 | 66.31 | N |
| ATOM | 8156 | CA | PHE | C | 284 | −40.944 | −11.101 | 89.659 | 1.00 | 60.36 | C |
| ATOM | 8157 | CB | PHE | C | 284 | −42.237 | −10.537 | 90.258 | 1.00 | 61.21 | C |
| ATOM | 8158 | CG | PHE | C | 284 | −43.114 | −9.825 | 89.273 | 1.00 | 62.48 | C |
| ATOM | 8159 | CD1 | PHE | C | 284 | −42.594 | −9.320 | 88.091 | 1.00 | 59.27 | C |
| ATOM | 8160 | CE1 | PHE | C | 284 | −43.412 | −8.662 | 87.190 | 1.00 | 56.96 | C |
| ATOM | 8161 | CZ | PHE | C | 284 | −44.763 | −8.497 | 87.465 | 1.00 | 58.41 | C |
| ATOM | 8162 | CE2 | PHE | C | 284 | −45.290 | −8.994 | 88.642 | 1.00 | 61.06 | C |
| ATOM | 8163 | CD2 | PHE | C | 284 | −44.465 | −9.652 | 89.537 | 1.00 | 62.25 | C |
| ATOM | 8164 | C | PHE | C | 284 | −39.971 | −11.243 | 90.790 | 1.00 | 62.52 | C |
| ATOM | 8165 | O | PHE | C | 284 | −39.092 | −10.418 | 90.954 | 1.00 | 69.55 | O |
| ATOM | 8166 | N | ILE | C | 285 | −40.116 | −12.319 | 91.550 | 1.00 | 63.01 | N |
| ATOM | 8167 | CA | ILE | C | 285 | −39.184 | −12.630 | 92.606 | 1.00 | 61.64 | C |
| ATOM | 8168 | CB | ILE | C | 285 | −39.575 | −13.924 | 93.336 | 1.00 | 56.63 | C |
| ATOM | 8169 | CG1 | ILE | C | 285 | −40.790 | −13.668 | 94.227 | 1.00 | 65.99 | C |
| ATOM | 8170 | CD1 | ILE | C | 285 | −41.342 | −14.918 | 94.882 | 1.00 | 73.38 | C |
| ATOM | 8171 | CG2 | ILE | C | 285 | −38.414 | −14.451 | 94.168 | 1.00 | 50.86 | C |
| ATOM | 8172 | C | ILE | C | 285 | −37.790 | −12.764 | 92.037 | 1.00 | 67.65 | C |
| ATOM | 8173 | O | ILE | C | 285 | −36.848 | −12.169 | 92.521 | 1.00 | 70.55 | O |
| ATOM | 8174 | N | PHE | C | 286 | −37.673 | −13.498 | 90.947 | 1.00 | 68.11 | N |
| ATOM | 8175 | CA | PHE | C | 286 | −36.388 | −13.621 | 90.286 | 1.00 | 71.48 | C |
| ATOM | 8176 | CB | PHE | C | 286 | −36.489 | −14.537 | 89.060 | 1.00 | 69.68 | C |
| ATOM | 8177 | CG | PHE | C | 286 | −36.722 | −15.980 | 89.388 | 1.00 | 67.86 | C |
| ATOM | 8178 | CD1 | PHE | C | 286 | −36.385 | −16.490 | 90.633 | 1.00 | 73.82 | C |
| ATOM | 8179 | CE1 | PHE | C | 286 | −36.600 | −17.821 | 90.932 | 1.00 | 74.22 | C |
| ATOM | 8180 | CZ | PHE | C | 286 | −37.155 | −18.658 | 89.983 | 1.00 | 81.98 | C |
| ATOM | 8181 | CE2 | PHE | C | 286 | −37.491 | −18.161 | 88.736 | 1.00 | 77.39 | C |
| ATOM | 8182 | CD2 | PHE | C | 286 | −37.273 | −16.831 | 88.444 | 1.00 | 67.36 | C |
| ATOM | 8183 | C | PHE | C | 286 | −35.850 | −12.280 | 89.825 | 1.00 | 72.88 | C |
| ATOM | 8184 | O | PHE | C | 286 | −34.693 | −11.991 | 89.999 | 1.00 | 78.02 | O |
| ATOM | 8185 | N | LEU | C | 287 | −36.693 | −11.457 | 89.235 | 1.00 | 69.21 | N |
| ATOM | 8186 | CA | LEU | C | 287 | −36.259 | −10.168 | 88.756 | 1.00 | 64.06 | C |
| ATOM | 8187 | CB | LEU | C | 287 | −37.401 | −9.440 | 88.049 | 1.00 | 63.44 | C |
| ATOM | 8188 | CG | LEU | C | 287 | −37.056 | −8.051 | 87.514 | 1.00 | 61.59 | C |
| ATOM | 8189 | CD1 | LEU | C | 287 | −35.931 | −8.134 | 86.492 | 1.00 | 57.50 | C |
| ATOM | 8190 | CD2 | LEU | C | 287 | −38.286 | −7.390 | 86.918 | 1.00 | 61.63 | C |
| ATOM | 8191 | C | LEU | C | 287 | −35.710 | −9.310 | 89.869 | 1.00 | 76.02 | C |
| ATOM | 8192 | O | LEU | C | 287 | −34.693 | −8.679 | 89.706 | 1.00 | 82.51 | O |
| ATOM | 8193 | N | PHE | C | 288 | −36.390 | −9.304 | 91.019 | 1.00 | 74.20 | N |
| ATOM | 8194 | CA | PHE | C | 288 | −35.931 | −8.546 | 92.182 | 1.00 | 77.53 | C |
| ATOM | 8195 | CB | PHE | C | 288 | −36.971 | −8.542 | 93.302 | 1.00 | 71.12 | C |
| ATOM | 8196 | CG | PHE | C | 288 | −36.551 | −7.744 | 94.504 | 1.00 | 86.34 | C |
| ATOM | 8197 | CD1 | PHE | C | 288 | −36.463 | −6.360 | 94.437 | 1.00 | 93.30 | C |
| ATOM | 8198 | CE1 | PHE | C | 288 | −36.070 | −5.620 | 95.538 | 1.00 | 93.58 | C |
| ATOM | 8199 | CZ | PHE | C | 288 | −35.758 | −6.263 | 96.724 | 1.00 | 96.84 | C |
| ATOM | 8200 | CE2 | PHE | C | 288 | −35.841 | −7.643 | 96.803 | 1.00 | 92.86 | C |
| ATOM | 8201 | CD2 | PHE | C | 288 | −36.233 | −8.375 | 95.697 | 1.00 | 87.76 | C |
| ATOM | 8202 | C | PHE | C | 288 | −34.602 | −9.075 | 92.697 | 1.00 | 81.97 | C |
| ATOM | 8203 | O | PHE | C | 288 | −33.671 | −8.329 | 92.928 | 1.00 | 81.26 | O |
| ATOM | 8204 | N | ASN | C | 289 | −34.517 | −10.388 | 92.797 | 1.00 | 84.31 | N |
| ATOM | 8205 | CA | ASN | C | 289 | −33.304 | −11.074 | 93.228 | 1.00 | 87.45 | C |
| ATOM | 8206 | CB | ASN | C | 289 | −33.568 | −12.579 | 93.331 | 1.00 | 88.69 | C |
| ATOM | 8207 | CG | ASN | C | 289 | −32.613 | −13.276 | 94.280 | 1.00 | 93.56 | C |
| ATOM | 8208 | OD1 | ASN | C | 289 | −31.400 | −13.062 | 94.233 | 1.00 | 92.16 | O |
| ATOM | 8209 | ND2 | ASN | C | 289 | −33.160 | −14.115 | 95.154 | 1.00 | 88.91 | N |
| ATOM | 8210 | C | ASN | C | 289 | −32.115 | −10.796 | 92.273 | 1.00 | 82.13 | C |
| ATOM | 8211 | O | ASN | C | 289 | −30.993 | −10.554 | 92.711 | 1.00 | 86.74 | O |
| ATOM | 8212 | N | ILE | C | 290 | −32.414 | −10.769 | 90.964 | 1.00 | 75.84 | N |
| ATOM | 8213 | CA | ILE | C | 290 | −31.468 | −10.397 | 89.920 | 1.00 | 78.16 | C |
| ATOM | 8214 | CB | ILE | C | 290 | −32.071 | −10.505 | 88.512 | 1.00 | 77.96 | C |
| ATOM | 8215 | CG1 | ILE | C | 290 | −32.314 | −11.972 | 88.158 | 1.00 | 74.52 | C |
| ATOM | 8216 | CD1 | ILE | C | 290 | −33.176 | −12.171 | 86.932 | 1.00 | 70.10 | C |
| ATOM | 8217 | CG2 | ILE | C | 290 | −31.119 | −9.917 | 87.498 | 1.00 | 77.89 | C |
| ATOM | 8218 | C | ILE | C | 290 | −30.869 | −9.015 | 90.153 | 1.00 | 82.27 | C |
| ATOM | 8219 | O | ILE | C | 290 | −29.670 | −8.873 | 90.288 | 1.00 | 85.73 | O |
| ATOM | 8220 | N | VAL | C | 291 | −31.719 | −7.993 | 90.228 | 1.00 | 77.37 | N |
| ATOM | 8221 | CA | VAL | C | 291 | −31.254 | −6.645 | 90.476 | 1.00 | 82.32 | C |
| ATOM | 8222 | CB | VAL | C | 291 | −32.446 | −5.655 | 90.485 | 1.00 | 70.95 | C |
| ATOM | 8223 | CG1 | VAL | C | 291 | −31.991 | −4.258 | 90.874 | 1.00 | 80.80 | C |
| ATOM | 8224 | CG2 | VAL | C | 291 | −33.147 | −5.639 | 89.132 | 1.00 | 67.73 | C |
| ATOM | 8225 | C | VAL | C | 291 | −30.511 | −6.542 | 91.803 | 1.00 | 90.02 | C |
| ATOM | 8226 | O | VAL | C | 291 | −29.526 | −5.830 | 91.907 | 1.00 | 90.65 | O |
| ATOM | 8227 | N | ARG | C | 292 | −30.988 | −7.262 | 92.830 | 1.00 | 90.46 | N |
| ATOM | 8228 | CA | ARG | C | 292 | −30.362 | −7.177 | 94.148 | 1.00 | 90.61 | C |
| ATOM | 8229 | CB | ARG | C | 292 | −31.130 | −8.001 | 95.185 | 1.00 | 92.47 | C |
| ATOM | 8230 | CG | ARG | C | 292 | −30.465 | −8.003 | 96.561 | 1.00 | 96.30 | C |
| ATOM | 8231 | CD | ARG | C | 292 | −31.150 | −8.944 | 97.556 | 1.00 | 96.78 | C |
| ATOM | 8232 | NE | ARG | C | 292 | −30.815 | −10.352 | 97.332 | 1.00 | 100.91 | N |
| ATOM | 8233 | CZ | ARG | C | 292 | −29.703 | −10.943 | 97.768 | 1.00 | 107.16 | C |
| ATOM | 8234 | NH1 | ARG | C | 292 | −28.799 | −10.251 | 98.450 | 1.00 | 107.03 | N |
| ATOM | 8235 | NH2 | ARG | C | 292 | −29.488 | −12.229 | 97.516 | 1.00 | 105.18 | N |
| ATOM | 8236 | C | ARG | C | 292 | −28.916 | −7.650 | 94.109 | 1.00 | 92.35 | C |
| ATOM | 8237 | O | ARG | C | 292 | −28.026 | −6.948 | 94.553 | 1.00 | 102.31 | O |
| ATOM | 8238 | N | ILE | C | 293 | −28.672 | −8.786 | 93.479 | 1.00 | 88.96 | N |
| ATOM | 8239 | CA | ILE | C | 293 | −27.306 | −9.223 | 93.231 | 1.00 | 91.71 | C |
| ATOM | 8240 | CB | ILE | C | 293 | −27.249 | −10.695 | 92.774 | 1.00 | 90.56 | C |
| ATOM | 8241 | CG1 | ILE | C | 293 | −27.658 | −11.598 | 93.941 | 1.00 | 94.44 | C |
| ATOM | 8242 | CD1 | ILE | C | 293 | −27.493 | −13.076 | 93.666 | 1.00 | 98.37 | C |
| ATOM | 8243 | CG2 | ILE | C | 293 | −25.854 | −11.069 | 92.296 | 1.00 | 80.88 | C |
| ATOM | 8244 | C | ILE | C | 293 | −26.530 | −8.279 | 92.292 | 1.00 | 98.93 | C |
| ATOM | 8245 | O | ILE | C | 293 | −25.353 | −8.009 | 92.503 | 1.00 | 102.08 | O |
| ATOM | 8246 | N | LEU | C | 294 | −27.175 | −7.836 | 91.203 | 1.00 | 94.96 | N |
| ATOM | 8247 | CA | LEU | C | 294 | −26.506 | −6.961 | 90.248 | 1.00 | 96.10 | C |
| ATOM | 8248 | CB | LEU | C | 294 | −27.426 | −6.640 | 89.069 | 1.00 | 86.44 | C |
| ATOM | 8249 | CG | LEU | C | 294 | −27.507 | −7.681 | 87.954 | 1.00 | 84.53 | C |
| ATOM | 8250 | CD1 | LEU | C | 294 | −28.316 | −7.142 | 86.790 | 1.00 | 90.91 | C |
| ATOM | 8251 | CD2 | LEU | C | 294 | −26.116 | −8.083 | 87.494 | 1.00 | 90.27 | C |
| ATOM | 8252 | C | LEU | C | 294 | −26.027 | −5.656 | 90.890 | 1.00 | 100.52 | C |
| ATOM | 8253 | O | LEU | C | 294 | −24.879 | −5.261 | 90.726 | 1.00 | 103.21 | O |
| ATOM | 8254 | N | MET | C | 295 | −26.873 | −5.012 | 91.685 | 1.00 | 98.13 | N |
| ATOM | 8255 | CA | MET | C | 295 | −26.409 | −3.825 | 92.368 | 1.00 | 101.37 | C |
| ATOM | 8256 | CB | MET | C | 295 | −27.589 | −3.065 | 92.976 | 1.00 | 92.47 | C |
| ATOM | 8257 | CG | MET | C | 295 | −28.454 | −2.363 | 91.951 | 1.00 | 93.69 | C |
| ATOM | 8258 | SD | MET | C | 295 | −29.830 | −1.469 | 92.689 | 1.00 | 92.21 | S |
| ATOM | 8259 | CE | MET | C | 295 | −30.469 | −0.624 | 91.246 | 1.00 | 93.25 | C |
| ATOM | 8260 | C | MET | C | 295 | −25.362 | −4.131 | 93.468 | 1.00 | 105.77 | C |
| ATOM | 8261 | O | MET | C | 295 | −24.291 | −3.538 | 93.521 | 1.00 | 109.71 | O |
| ATOM | 8262 | N | THR | C | 296 | −25.755 | −4.975 | 94.432 | 1.00 | 105.45 | N |
| ATOM | 8263 | CA | THR | C | 296 | −24.897 | −5.210 | 95.581 | 1.00 | 106.28 | C |
| ATOM | 8264 | CB | THR | C | 296 | −25.627 | −6.016 | 96.681 | 1.00 | 111.55 | C |
| ATOM | 8265 | OG1 | THR | C | 296 | −26.055 | −7.278 | 96.154 | 1.00 | 118.72 | O |
| ATOM | 8266 | CG2 | THR | C | 296 | −26.838 | −5.246 | 97.190 | 1.00 | 107.90 | C |
| ATOM | 8267 | C | THR | C | 296 | −23.531 | −5.858 | 95.304 | 1.00 | 98.87 | C |
| ATOM | 8268 | O | THR | C | 296 | −22.517 | −5.397 | 95.820 | 1.00 | 101.76 | O |
| ATOM | 8269 | N | LYS | C | 297 | −23.550 | −7.055 | 94.707 | 1.00 | 95.87 | N |
| ATOM | 8270 | CA | LYS | C | 297 | −22.340 | −7.870 | 94.708 | 1.00 | 103.63 | C |
| ATOM | 8271 | CB | LYS | C | 297 | −22.668 | −9.317 | 95.091 | 1.00 | 104.69 | C |
| ATOM | 8272 | CG | LYS | C | 297 | −23.369 | −9.475 | 96.430 | 1.00 | 103.46 | C |
| ATOM | 8273 | CD | LYS | C | 297 | −23.588 | −10.944 | 96.758 | 1.00 | 107.41 | C |
| ATOM | 8274 | CE | LYS | C | 297 | −24.389 | −11.115 | 98.039 | 1.00 | 118.35 | C |
| ATOM | 8275 | NZ | LYS | C | 297 | −24.546 | −12.551 | 98.408 | 1.00 | 124.32 | N |
| ATOM | 8276 | C | LYS | C | 297 | −21.617 | −7.854 | 93.384 | 1.00 | 104.56 | C |
| ATOM | 8277 | O | LYS | C | 297 | −20.705 | −8.655 | 93.161 | 1.00 | 104.51 | O |
| ATOM | 8278 | N | LEU | C | 298 | −22.131 | −7.040 | 92.464 | 1.00 | 105.10 | N |
| ATOM | 8279 | CA | LEU | C | 298 | −21.600 | −7.024 | 91.121 | 1.00 | 107.43 | C |
| ATOM | 8280 | CB | LEU | C | 298 | −22.480 | −7.866 | 90.189 | 1.00 | 108.53 | C |
| ATOM | 8281 | CG | LEU | C | 298 | −22.603 | −9.367 | 90.474 | 1.00 | 105.22 | C |
| ATOM | 8282 | CD1 | LEU | C | 298 | −23.629 | −10.004 | 89.547 | 1.00 | 98.30 | C |
| ATOM | 8283 | CD2 | LEU | C | 298 | −21.257 | −10.077 | 90.355 | 1.00 | 86.84 | C |
| ATOM | 8284 | C | LEU | C | 298 | −21.449 | −5.615 | 90.562 | 1.00 | 112.05 | C |
| ATOM | 8285 | O | LEU | C | 298 | −21.277 | −5.479 | 89.362 | 1.00 | 115.66 | O |
| ATOM | 8286 | N | ARG | C | 299 | −21.688 | −4.565 | 91.382 | 1.00 | 111.52 | N |
| ATOM | 8287 | CA | ARG | C | 299 | −21.763 | −3.181 | 90.832 | 1.00 | 116.07 | C |
| ATOM | 8288 | CB | ARG | C | 299 | −21.910 | −2.154 | 91.960 | 1.00 | 106.50 | C |
| ATOM | 8289 | CG | ARG | C | 299 | −22.170 | −0.736 | 91.475 | 1.00 | 106.83 | C |
| ATOM | 8290 | CD | ARG | C | 299 | −22.600 | 0.184 | 92.612 | 1.00 | 116.36 | C |
| ATOM | 8291 | NE | ARG | C | 299 | −21.505 | 0.487 | 93.533 | 1.00 | 119.42 | N |
| ATOM | 8292 | CZ | ARG | C | 299 | −21.586 | 1.370 | 94.525 | 1.00 | 113.19 | C |
| ATOM | 8293 | NH1 | ARG | C | 299 | −22.714 | 2.037 | 94.726 | 1.00 | 104.33 | N |
| ATOM | 8294 | NH2 | ARG | C | 299 | −20.543 | 1.591 | 95.316 | 1.00 | 107.91 | N |
| ATOM | 8295 | C | ARG | C | 299 | −20.566 | −2.814 | 89.918 | 1.00 | 122.86 | C |
| ATOM | 8296 | O | ARG | C | 299 | −20.752 | −2.312 | 88.809 | 1.00 | 119.00 | O |
| ATOM | 8297 | N | ALA | C | 300 | −19.341 | −3.116 | 90.402 | 1.00 | 126.40 | N |
| ATOM | 8298 | CA | ALA | C | 300 | −18.115 | −2.869 | 89.637 | 1.00 | 124.59 | C |
| ATOM | 8299 | CB | ALA | C | 300 | −17.018 | −2.350 | 90.553 | 1.00 | 119.59 | C |
| ATOM | 8300 | C | ALA | C | 300 | −17.622 | −4.112 | 88.857 | 1.00 | 123.32 | C |
| ATOM | 8301 | O | ALA | C | 300 | −16.637 | −4.070 | 88.124 | 1.00 | 127.45 | O |
| ATOM | 8302 | N | SER | C | 301 | −18.311 | −5.228 | 88.992 | 1.00 | 121.67 | N |
| ATOM | 8303 | CA | SER | C | 301 | −17.852 | −6.407 | 88.294 | 1.00 | 127.92 | C |
| ATOM | 8304 | CB | SER | C | 301 | −18.426 | −7.674 | 88.932 | 1.00 | 129.33 | C |
| ATOM | 8305 | OG | SER | C | 301 | −19.775 | −7.873 | 88.545 | 1.00 | 123.91 | O |
| ATOM | 8306 | C | SER | C | 301 | −18.270 | −6.336 | 86.824 | 1.00 | 128.01 | C |
| ATOM | 8307 | O | SER | C | 301 | −19.415 | −6.052 | 86.504 | 1.00 | 130.73 | O |
| ATOM | 8308 | N | THR | C | 302 | −17.313 | −6.551 | 85.941 | 1.00 | 129.64 | N |
| ATOM | 8309 | CA | THR | C | 302 | −17.555 | −6.429 | 84.514 | 1.00 | 133.73 | C |
| ATOM | 8310 | CB | THR | C | 302 | −16.932 | −5.142 | 83.942 | 1.00 | 130.44 | C |
| ATOM | 8311 | OG1 | THR | C | 302 | −15.565 | −5.044 | 84.361 | 1.00 | 130.97 | O |
| ATOM | 8312 | CG2 | THR | C | 302 | −17.688 | −3.918 | 84.431 | 1.00 | 129.73 | C |
| ATOM | 8313 | C | THR | C | 302 | −16.954 | −7.643 | 83.811 | 1.00 | 139.59 | C |
| ATOM | 8314 | O | THR | C | 302 | −15.860 | −8.093 | 84.140 | 1.00 | 145.25 | O |
| ATOM | 8315 | O | THR | C | 303 | −19.331 | −8.970 | 80.895 | 1.00 | 139.18 | O |
| ATOM | 8316 | N | THR | C | 303 | −17.738 | −8.224 | 82.913 | 1.00 | 138.13 | N |
| ATOM | 8317 | CA | THR | C | 303 | −17.376 | −9.468 | 82.256 | 1.00 | 138.02 | C |
| ATOM | 8318 | C | THR | C | 303 | −18.311 | −9.652 | 81.034 | 1.00 | 138.57 | C |
| ATOM | 8319 | CB | THR | C | 303 | −17.686 | −10.734 | 83.129 | 1.00 | 136.67 | C |
| ATOM | 8320 | OG1 | THR | C | 303 | −19.094 | −10.809 | 83.391 | 1.00 | 137.81 | O |
| ATOM | 8321 | CG2 | THR | C | 303 | −16.925 | −10.781 | 84.457 | 1.00 | 127.31 | C |
| ATOM | 8322 | O | SER | C | 304 | −21.332 | −10.892 | 79.042 | 1.00 | 135.85 | O |
| ATOM | 8323 | N | SER | C | 304 | −18.025 | −10.675 | 80.225 | 1.00 | 136.92 | N |
| ATOM | 8324 | CA | SER | C | 304 | −18.897 | −10.993 | 79.105 | 1.00 | 136.66 | C |
| ATOM | 8325 | C | SER | C | 304 | −20.326 | −11.358 | 79.591 | 1.00 | 137.78 | C |
| ATOM | 8326 | CB | SER | C | 304 | −18.310 | −12.135 | 78.271 | 1.00 | 136.78 | C |
| ATOM | 8327 | OG | SER | C | 304 | −18.084 | −13.288 | 79.063 | 1.00 | 137.20 | O |
| ATOM | 8328 | N | GLU | C | 305 | −20.383 | −12.259 | 80.596 | 1.00 | 138.69 | N |
| ATOM | 8329 | CA | GLU | C | 305 | −21.663 | −12.732 | 81.127 | 1.00 | 132.90 | C |
| ATOM | 8330 | CB | GLU | C | 305 | −21.473 | −13.969 | 82.012 | 1.00 | 132.05 | C |
| ATOM | 8331 | CG | GLU | C | 305 | −22.782 | −14.603 | 82.467 | 1.00 | 129.63 | C |
| ATOM | 8332 | CD | GLU | C | 305 | −22.571 | −15.880 | 83.257 | 1.00 | 123.02 | C |
| ATOM | 8333 | OE1 | GLU | C | 305 | −21.403 | −16.246 | 83.505 | 1.00 | 121.51 | O |
| ATOM | 8334 | OE2 | GLU | C | 305 | −23.576 | −16.521 | 83.629 | 1.00 | 111.70 | O |
| ATOM | 8335 | C | GLU | C | 305 | −22.492 | −11.668 | 81.870 | 1.00 | 129.42 | C |
| ATOM | 8336 | O | GLU | C | 305 | −23.695 | −11.553 | 81.641 | 1.00 | 130.21 | O |
| ATOM | 8337 | N | THR | C | 306 | −21.819 | −10.827 | 82.684 | 1.00 | 128.10 | N |
| ATOM | 8338 | CA | THR | C | 306 | −22.488 | −9.680 | 83.330 | 1.00 | 127.19 | C |
| ATOM | 8339 | CB | THR | C | 306 | −21.539 | −8.908 | 84.267 | 1.00 | 130.38 | C |
| ATOM | 8340 | OG1 | THR | C | 306 | −20.284 | −8.698 | 83.609 | 1.00 | 140.97 | O |
| ATOM | 8341 | CG2 | THR | C | 306 | −21.302 | −9.695 | 85.549 | 1.00 | 121.68 | C |
| ATOM | 8342 | C | THR | C | 306 | −23.129 | −8.725 | 82.299 | 1.00 | 124.79 | C |
| ATOM | 8343 | O | THR | C | 306 | −24.258 | −8.275 | 82.450 | 1.00 | 116.83 | O |
| ATOM | 8344 | N | ILE | C | 307 | −22.383 | −8.436 | 81.227 | 1.00 | 131.35 | N |
| ATOM | 8345 | CA | ILE | C | 307 | −22.882 | −7.558 | 80.173 | 1.00 | 131.76 | C |
| ATOM | 8346 | CB | ILE | C | 307 | −21.839 | −7.345 | 79.061 | 1.00 | 131.67 | C |
| ATOM | 8347 | CG1 | ILE | C | 307 | −20.716 | −6.440 | 79.570 | 1.00 | 127.61 | C |
| ATOM | 8348 | CD1 | ILE | C | 307 | −19.614 | −6.206 | 78.556 | 1.00 | 128.51 | C |
| ATOM | 8349 | CG2 | ILE | C | 307 | −22.479 | −6.720 | 77.830 | 1.00 | 135.86 | C |
| ATOM | 8350 | C | ILE | C | 307 | −24.222 | −8.079 | 79.581 | 1.00 | 127.60 | C |
| ATOM | 8351 | O | ILE | C | 307 | −25.197 | −7.340 | 79.505 | 1.00 | 121.17 | O |
| ATOM | 8352 | N | GLN | C | 308 | −24.316 | −9.379 | 79.301 | 1.00 | 127.23 | N |
| ATOM | 8353 | CA | GLN | C | 308 | −25.602 | −9.929 | 78.891 | 1.00 | 123.55 | C |
| ATOM | 8354 | CB | GLN | C | 308 | −25.452 | −11.387 | 78.460 | 1.00 | 121.41 | C |
| ATOM | 8355 | CG | GLN | C | 308 | −24.593 | −11.583 | 77.230 | 1.00 | 124.06 | C |
| ATOM | 8356 | CD | GLN | C | 308 | −24.556 | −13.029 | 76.784 | 1.00 | 132.17 | C |
| ATOM | 8357 | OE1 | GLN | C | 308 | −25.217 | −13.407 | 75.816 | 1.00 | 126.47 | O |
| ATOM | 8358 | NE2 | GLN | C | 308 | −23.783 | −13.848 | 77.490 | 1.00 | 133.53 | N |
| ATOM | 8359 | C | GLN | C | 308 | −26.660 | −9.823 | 80.022 | 1.00 | 116.15 | C |
| ATOM | 8360 | O | GLN | C | 308 | −27.839 | −9.579 | 79.782 | 1.00 | 107.02 | O |
| ATOM | 8361 | N | ALA | C | 309 | −26.227 | −10.025 | 81.268 | 1.00 | 120.48 | N |
| ATOM | 8362 | CA | ALA | C | 309 | −27.145 | −9.919 | 82.410 | 1.00 | 112.30 | C |
| ATOM | 8363 | CB | ALA | C | 309 | −26.424 | −10.279 | 83.697 | 1.00 | 112.55 | C |
| ATOM | 8364 | C | ALA | C | 309 | −27.823 | −8.532 | 82.537 | 1.00 | 107.77 | C |
| ATOM | 8365 | O | ALA | C | 309 | −29.032 | −8.430 | 82.709 | 1.00 | 103.92 | O |
| ATOM | 8366 | N | ARG | C | 310 | −27.020 | −7.466 | 82.403 | 1.00 | 114.22 | N |
| ATOM | 8367 | CA | ARG | C | 310 | −27.524 | −6.089 | 82.455 | 1.00 | 110.27 | C |
| ATOM | 8368 | CB | ARG | C | 310 | −26.373 | −5.078 | 82.434 | 1.00 | 115.08 | C |
| ATOM | 8369 | CG | ARG | C | 310 | −25.660 | −4.883 | 83.757 | 1.00 | 113.80 | C |
| ATOM | 8370 | CD | ARG | C | 310 | −24.696 | −3.726 | 83.666 | 1.00 | 116.14 | C |
| ATOM | 8371 | NE | ARG | C | 310 | −23.929 | −3.538 | 84.887 | 1.00 | 109.49 | N |
| ATOM | 8372 | CZ | ARG | C | 310 | −22.733 | −4.071 | 85.097 | 1.00 | 116.66 | C |
| ATOM | 8373 | NH1 | ARG | C | 310 | −22.175 | −4.842 | 84.170 | 1.00 | 116.20 | N |
| ATOM | 8374 | NH2 | ARG | C | 310 | −22.097 | −3.834 | 86.235 | 1.00 | 120.06 | N |
| ATOM | 8375 | C | ARG | C | 310 | −28.471 | −5.772 | 81.283 | 1.00 | 101.54 | C |
| ATOM | 8376 | O | ARG | C | 310 | −29.517 | −5.156 | 81.457 | 1.00 | 92.04 | O |
| ATOM | 8377 | N | LYS | C | 311 | −28.100 | −6.211 | 80.071 | 1.00 | 101.94 | N |
| ATOM | 8378 | CA | LYS | C | 311 | −28.965 | −5.990 | 78.939 | 1.00 | 97.95 | C |
| ATOM | 8379 | CB | LYS | C | 311 | −28.284 | −6.381 | 77.620 | 1.00 | 93.91 | C |
| ATOM | 8380 | CG | LYS | C | 311 | −27.313 | −5.317 | 77.108 | 1.00 | 98.10 | C |
| ATOM | 8381 | CD | LYS | C | 311 | −27.320 | −5.206 | 75.584 | 1.00 | 102.03 | C |
| ATOM | 8382 | CE | LYS | C | 311 | −26.625 | −3.925 | 75.117 | 1.00 | 95.72 | C |
| ATOM | 8383 | NZ | LYS | C | 311 | −26.586 | −3.802 | 73.631 | 1.00 | 81.69 | N |
| ATOM | 8384 | C | LYS | C | 311 | −30.323 | −6.710 | 79.125 | 1.00 | 94.98 | C |
| ATOM | 8385 | O | LYS | C | 311 | −31.390 | −6.163 | 78.866 | 1.00 | 86.05 | O |
| ATOM | 8386 | N | ALA | C | 312 | −30.272 | −7.950 | 79.592 | 1.00 | 95.39 | N |
| ATOM | 8387 | CA | ALA | C | 312 | −31.483 | −8.716 | 79.795 | 1.00 | 85.89 | C |
| ATOM | 8388 | CB | ALA | C | 312 | −31.142 | −10.135 | 80.230 | 1.00 | 87.92 | C |
| ATOM | 8389 | C | ALA | C | 312 | −32.434 | −8.068 | 80.793 | 1.00 | 80.24 | C |
| ATOM | 8390 | O | ALA | C | 312 | −33.607 | −7.885 | 80.520 | 1.00 | 80.81 | O |
| ATOM | 8391 | N | VAL | C | 313 | −31.908 | −7.627 | 81.914 | 1.00 | 75.08 | N |
| ATOM | 8392 | CA | VAL | C | 313 | −32.719 | −6.905 | 82.868 | 1.00 | 70.80 | C |
| ATOM | 8393 | CB | VAL | C | 313 | −31.914 | −6.556 | 84.136 | 1.00 | 68.94 | C |
| ATOM | 8394 | CG1 | VAL | C | 313 | −32.785 | −5.829 | 85.152 | 1.00 | 63.53 | C |
| ATOM | 8395 | CG2 | VAL | C | 313 | −31.337 | −7.807 | 84.742 | 1.00 | 66.96 | C |
| ATOM | 8396 | C | VAL | C | 313 | −33.284 | −5.602 | 82.262 | 1.00 | 76.07 | C |
| ATOM | 8397 | O | VAL | C | 313 | −34.432 | −5.252 | 82.493 | 1.00 | 70.42 | O |
| ATOM | 8398 | N | LYS | C | 314 | −32.450 | −4.860 | 81.517 | 1.00 | 80.13 | N |
| ATOM | 8399 | CA | LYS | C | 314 | −32.909 | −3.627 | 80.884 | 1.00 | 80.89 | C |
| ATOM | 8400 | CB | LYS | C | 314 | −31.797 | −3.008 | 80.036 | 1.00 | 86.25 | C |
| ATOM | 8401 | CG | LYS | C | 314 | −30.668 | −2.363 | 80.816 | 1.00 | 86.41 | C |
| ATOM | 8402 | CD | LYS | C | 314 | −29.579 | −1.892 | 79.864 | 1.00 | 91.83 | C |
| ATOM | 8403 | CE | LYS | C | 314 | −28.385 | −1.319 | 80.608 | 1.00 | 111.13 | C |
| ATOM | 8404 | NZ | LYS | C | 314 | −27.314 | −0.862 | 79.674 | 1.00 | 112.99 | N |
| ATOM | 8405 | C | LYS | C | 314 | −34.134 | −3.863 | 79.991 | 1.00 | 80.69 | C |
| ATOM | 8406 | O | LYS | C | 314 | −35.153 | −3.189 | 80.120 | 1.00 | 78.34 | O |
| ATOM | 8407 | N | ALA | C | 315 | −34.016 | −4.858 | 79.109 | 1.00 | 75.35 | N |
| ATOM | 8408 | CA | ALA | C | 315 | −35.103 | −5.259 | 78.238 | 1.00 | 73.93 | C |
| ATOM | 8409 | CB | ALA | C | 315 | −34.633 | −6.354 | 77.290 | 1.00 | 77.47 | C |
| ATOM | 8410 | C | ALA | C | 315 | −36.377 | −5.706 | 78.981 | 1.00 | 73.08 | C |
| ATOM | 8411 | O | ALA | C | 315 | −37.479 | −5.320 | 78.638 | 1.00 | 70.13 | O |
| ATOM | 8412 | N | THR | C | 316 | −36.218 | −6.548 | 80.001 | 1.00 | 71.68 | N |
| ATOM | 8413 | CA | THR | C | 316 | −37.363 | −7.033 | 80.771 | 1.00 | 66.20 | C |
| ATOM | 8414 | CB | THR | C | 316 | −36.882 | −8.001 | 81.864 | 1.00 | 60.35 | C |
| ATOM | 8415 | OG1 | THR | C | 316 | −36.363 | −9.191 | 81.258 | 1.00 | 61.94 | O |
| ATOM | 8416 | CG2 | THR | C | 316 | −38.016 | −8.359 | 82.808 | 1.00 | 62.71 | C |
| ATOM | 8417 | C | THR | C | 316 | −38.116 | −5.904 | 81.453 | 1.00 | 70.85 | C |
| ATOM | 8418 | O | THR | C | 316 | −39.334 | −5.855 | 81.454 | 1.00 | 69.22 | O |
| ATOM | 8419 | N | LEU | C | 317 | −37.353 | −4.968 | 82.004 | 1.00 | 71.07 | N |
| ATOM | 8420 | CA | LEU | C | 317 | −37.913 | −3.765 | 82.575 | 1.00 | 70.55 | C |
| ATOM | 8421 | CB | LEU | C | 317 | −36.819 | −2.897 | 83.202 | 1.00 | 75.32 | C |
| ATOM | 8422 | CG | LEU | C | 317 | −36.187 | −3.450 | 84.483 | 1.00 | 76.73 | C |
| ATOM | 8423 | CD1 | LEU | C | 317 | −35.042 | −2.562 | 84.946 | 1.00 | 86.51 | C |
| ATOM | 8424 | CD2 | LEU | C | 317 | −37.226 | −3.602 | 85.585 | 1.00 | 71.14 | C |
| ATOM | 8425 | C | LEU | C | 317 | −38.732 | −2.962 | 81.552 | 1.00 | 67.77 | C |
| ATOM | 8426 | O | LEU | C | 317 | −39.819 | −2.513 | 81.859 | 1.00 | 71.62 | O |
| ATOM | 8427 | N | VAL | C | 318 | −38.237 | −2.798 | 80.328 | 1.00 | 64.83 | N |
| ATOM | 8428 | CA | VAL | C | 318 | −39.037 | −2.101 | 79.321 | 1.00 | 65.44 | C |
| ATOM | 8429 | CB | VAL | C | 318 | −38.203 | −1.804 | 78.061 | 1.00 | 67.72 | C |
| ATOM | 8430 | CG1 | VAL | C | 318 | −39.088 | −1.295 | 76.927 | 1.00 | 52.03 | C |
| ATOM | 8431 | CG2 | VAL | C | 318 | −37.101 | −0.807 | 78.386 | 1.00 | 77.97 | C |
| ATOM | 8432 | C | VAL | C | 318 | −40.301 | −2.882 | 78.910 | 1.00 | 67.43 | C |
| ATOM | 8433 | O | VAL | C | 318 | −41.377 | −2.317 | 78.770 | 1.00 | 63.64 | O |
| ATOM | 8434 | N | LEU | C | 319 | −40.107 | −4.181 | 78.637 | 1.00 | 67.10 | N |
| ATOM | 8435 | CA | LEU | C | 319 | −41.149 | −5.067 | 78.100 | 1.00 | 64.31 | C |
| ATOM | 8436 | CB | LEU | C | 319 | −40.533 | −6.403 | 77.677 | 1.00 | 64.03 | C |
| ATOM | 8437 | CG | LEU | C | 319 | −41.439 | −7.374 | 76.921 | 1.00 | 61.87 | C |
| ATOM | 8438 | CD1 | LEU | C | 319 | −41.998 | −6.729 | 75.658 | 1.00 | 55.55 | C |
| ATOM | 8439 | CD2 | LEU | C | 319 | −40.680 | −8.647 | 76.585 | 1.00 | 65.13 | C |
| ATOM | 8440 | C | LEU | C | 319 | −42.331 | −5.310 | 79.024 | 1.00 | 65.77 | C |
| ATOM | 8441 | O | LEU | C | 319 | −43.464 | −5.482 | 78.597 | 1.00 | 66.19 | O |
| ATOM | 8442 | N | LEU | C | 320 | −42.025 | −5.466 | 80.296 | 1.00 | 66.07 | N |
| ATOM | 8443 | CA | LEU | C | 320 | −43.042 | −5.788 | 81.265 | 1.00 | 67.07 | C |
| ATOM | 8444 | CB | LEU | C | 320 | −42.413 | −5.987 | 82.650 | 1.00 | 62.29 | C |
| ATOM | 8445 | CG | LEU | C | 320 | −43.359 | −6.250 | 83.820 | 1.00 | 56.22 | C |
| ATOM | 8446 | CD1 | LEU | C | 320 | −43.941 | −7.653 | 83.742 | 1.00 | 52.65 | C |
| ATOM | 8447 | CD2 | LEU | C | 320 | −42.633 | −6.026 | 85.133 | 1.00 | 49.37 | C |
| ATOM | 8448 | C | LEU | C | 320 | −44.166 | −4.760 | 81.309 | 1.00 | 65.37 | C |
| ATOM | 8449 | O | LEU | C | 320 | −45.315 | −5.138 | 81.242 | 1.00 | 61.54 | O |
| ATOM | 8450 | N | PRO | C | 321 | −43.951 | −3.456 | 81.310 | 1.00 | 70.12 | N |
| ATOM | 8451 | CA | PRO | C | 321 | −45.043 | −2.503 | 81.201 | 1.00 | 70.48 | C |
| ATOM | 8452 | CB | PRO | C | 321 | −44.334 | −1.150 | 81.263 | 1.00 | 82.90 | C |
| ATOM | 8453 | CG | PRO | C | 321 | −43.139 | −1.412 | 82.079 | 1.00 | 82.50 | C |
| ATOM | 8454 | CD | PRO | C | 321 | −42.695 | −2.773 | 81.646 | 1.00 | 70.25 | C |
| ATOM | 8455 | C | PRO | C | 321 | −45.840 | −2.626 | 79.894 | 1.00 | 71.19 | C |
| ATOM | 8456 | O | PRO | C | 321 | −47.042 | −2.423 | 79.956 | 1.00 | 76.75 | O |
| ATOM | 8457 | N | LEU | C | 322 | −45.208 | −2.828 | 78.719 | 1.00 | 61.99 | N |
| ATOM | 8458 | CA | LEU | C | 322 | −45.968 | −2.823 | 77.464 | 1.00 | 60.87 | C |
| ATOM | 8459 | CB | LEU | C | 322 | −45.023 | −2.922 | 76.268 | 1.00 | 56.43 | C |
| ATOM | 8460 | CG | LEU | C | 322 | −44.106 | −1.744 | 75.952 | 1.00 | 53.11 | C |
| ATOM | 8461 | CD1 | LEU | C | 322 | −43.099 | −2.156 | 74.894 | 1.00 | 48.74 | C |
| ATOM | 8462 | CD2 | LEU | C | 322 | −44.914 | −0.547 | 75.481 | 1.00 | 44.96 | C |
| ATOM | 8463 | C | LEU | C | 322 | −46.983 | −3.981 | 77.396 | 1.00 | 73.58 | C |
| ATOM | 8464 | O | LEU | C | 322 | −48.189 | −3.807 | 77.166 | 1.00 | 79.42 | O |
| ATOM | 8465 | N | LEU | C | 323 | −46.492 | −5.180 | 77.707 | 1.00 | 68.82 | N |
| ATOM | 8466 | CA | LEU | C | 323 | −47.363 | −6.325 | 77.841 | 1.00 | 66.79 | C |
| ATOM | 8467 | CB | LEU | C | 323 | −46.537 | −7.598 | 78.037 | 1.00 | 62.41 | C |
| ATOM | 8468 | CG | LEU | C | 323 | −45.372 | −7.754 | 77.055 | 1.00 | 56.88 | C |
| ATOM | 8469 | CD1 | LEU | C | 323 | −44.671 | −9.086 | 77.253 | 1.00 | 57.59 | C |
| ATOM | 8470 | CD2 | LEU | C | 323 | −45.841 | −7.605 | 75.616 | 1.00 | 58.92 | C |
| ATOM | 8471 | C | LEU | C | 323 | −48.379 | −6.147 | 78.974 | 1.00 | 66.91 | C |
| ATOM | 8472 | O | LEU | C | 323 | −49.559 | −6.420 | 78.833 | 1.00 | 72.58 | O |
| ATOM | 8473 | N | GLY | C | 324 | −47.864 | −5.745 | 80.127 | 1.00 | 62.81 | N |
| ATOM | 8474 | CA | GLY | C | 324 | −48.592 | −5.638 | 81.356 | 1.00 | 68.90 | C |
| ATOM | 8475 | C | GLY | C | 324 | −49.742 | −4.697 | 81.367 | 1.00 | 72.63 | C |
| ATOM | 8476 | O | GLY | C | 324 | −50.776 | −5.037 | 81.932 | 1.00 | 73.07 | O |
| ATOM | 8477 | N | ILE | C | 325 | −49.616 | −3.536 | 80.716 | 1.00 | 68.67 | N |
| ATOM | 8478 | CA | ILE | C | 325 | −50.751 | −2.636 | 80.541 | 1.00 | 70.87 | C |
| ATOM | 8479 | CB | ILE | C | 325 | −50.370 | −1.295 | 79.860 | 1.00 | 66.98 | C |
| ATOM | 8480 | CG1 | ILE | C | 325 | −51.570 | −0.343 | 79.841 | 1.00 | 76.86 | C |
| ATOM | 8481 | CD1 | ILE | C | 325 | −51.237 | 1.062 | 79.354 | 1.00 | 76.01 | C |
| ATOM | 8482 | CG2 | ILE | C | 325 | −49.824 | −1.515 | 78.468 | 1.00 | 65.03 | C |
| ATOM | 8483 | C | ILE | C | 325 | −51.910 | −3.354 | 79.858 | 1.00 | 71.62 | C |
| ATOM | 8484 | O | ILE | C | 325 | −53.058 | −3.206 | 80.249 | 1.00 | 70.16 | O |
| ATOM | 8485 | N | THR | C | 326 | −51.598 | −4.137 | 78.818 | 1.00 | 74.69 | N |
| ATOM | 8486 | CA | THR | C | 326 | −52.651 | −4.858 | 78.101 | 1.00 | 71.69 | C |
| ATOM | 8487 | CB | THR | C | 326 | −52.062 | −5.739 | 76.978 | 1.00 | 72.79 | C |
| ATOM | 8488 | OG1 | THR | C | 326 | −51.286 | −4.937 | 76.077 | 1.00 | 74.04 | O |
| ATOM | 8489 | CG2 | THR | C | 326 | −53.175 | −6.439 | 76.214 | 1.00 | 69.90 | C |
| ATOM | 8490 | C | THR | C | 326 | −53.477 | −5.776 | 79.015 | 1.00 | 72.21 | C |
| ATOM | 8491 | O | THR | C | 326 | −54.700 | −5.767 | 79.003 | 1.00 | 75.27 | O |
| ATOM | 8492 | N | TYR | C | 327 | −52.789 | −6.503 | 79.869 | 1.00 | 69.23 | N |
| ATOM | 8493 | CA | TYR | C | 327 | −53.457 | −7.377 | 80.816 | 1.00 | 67.28 | C |
| ATOM | 8494 | CB | TYR | C | 327 | −52.478 | −8.343 | 81.491 | 1.00 | 66.33 | C |
| ATOM | 8495 | CG | TYR | C | 327 | −51.923 | −9.369 | 80.517 | 1.00 | 70.57 | C |
| ATOM | 8496 | CD1 | TYR | C | 327 | −52.575 | −10.580 | 80.298 | 1.00 | 66.50 | C |
| ATOM | 8497 | CE1 | TYR | C | 327 | −52.076 | −11.512 | 79.393 | 1.00 | 59.01 | C |
| ATOM | 8498 | CZ | TYR | C | 327 | −50.922 | −11.234 | 78.692 | 1.00 | 55.91 | C |
| ATOM | 8499 | OH | TYR | C | 327 | −50.426 | −12.152 | 77.794 | 1.00 | 54.99 | O |
| ATOM | 8500 | CE2 | TYR | C | 327 | −50.262 | −10.037 | 78.886 | 1.00 | 62.47 | C |
| ATOM | 8501 | CD2 | TYR | C | 327 | −50.765 | −9.113 | 79.792 | 1.00 | 68.47 | C |
| ATOM | 8502 | C | TYR | C | 327 | −54.332 | −6.597 | 81.813 | 1.00 | 72.35 | C |
| ATOM | 8503 | O | TYR | C | 327 | −55.442 | −7.002 | 82.146 | 1.00 | 77.80 | O |
| ATOM | 8504 | N | MET | C | 328 | −53.838 | −5.459 | 82.298 | 1.00 | 72.76 | N |
| ATOM | 8505 | CA | MET | C | 328 | −54.624 | −4.663 | 83.259 | 1.00 | 73.07 | C |
| ATOM | 8506 | CB | MET | C | 328 | −53.828 | −3.429 | 83.692 | 1.00 | 75.86 | C |
| ATOM | 8507 | CG | MET | C | 328 | −52.638 | −3.705 | 84.597 | 1.00 | 82.86 | C |
| ATOM | 8508 | SD | MET | C | 328 | −53.136 | −3.979 | 86.305 | 1.00 | 100.73 | S |
| ATOM | 8509 | CE | MET | C | 328 | −54.378 | −2.701 | 86.498 | 1.00 | 86.09 | C |
| ATOM | 8510 | C | MET | C | 328 | −55.942 | −4.203 | 82.640 | 1.00 | 75.63 | C |
| ATOM | 8511 | O | MET | C | 328 | −57.014 | −4.336 | 83.213 | 1.00 | 79.57 | O |
| ATOM | 8512 | N | LEU | C | 329 | −55.826 | −3.726 | 81.413 | 1.00 | 74.62 | N |
| ATOM | 8513 | CA | LEU | C | 329 | −56.948 | −3.338 | 80.609 | 1.00 | 70.85 | C |
| ATOM | 8514 | CB | LEU | C | 329 | −56.494 | −2.878 | 79.223 | 1.00 | 74.19 | C |
| ATOM | 8515 | CG | LEU | C | 329 | −55.678 | −1.584 | 79.237 | 1.00 | 71.39 | C |
| ATOM | 8516 | CD1 | LEU | C | 329 | −55.297 | −1.164 | 77.826 | 1.00 | 76.29 | C |
| ATOM | 8517 | CD2 | LEU | C | 329 | −56.444 | −0.477 | 79.947 | 1.00 | 75.45 | C |
| ATOM | 8518 | C | LEU | C | 329 | −57.985 | −4.431 | 80.508 | 1.00 | 78.28 | C |
| ATOM | 8519 | O | LEU | C | 329 | −59.146 | −4.182 | 80.706 | 1.00 | 85.11 | O |
| ATOM | 8520 | N | ALA | C | 330 | −57.586 | −5.664 | 80.332 | 1.00 | 78.80 | N |
| ATOM | 8521 | CA | ALA | C | 330 | −58.604 | −6.681 | 80.222 | 1.00 | 77.93 | C |
| ATOM | 8522 | CB | ALA | C | 330 | −57.974 | −8.024 | 79.869 | 1.00 | 77.53 | C |
| ATOM | 8523 | C | ALA | C | 330 | −59.468 | −6.814 | 81.462 | 1.00 | 83.04 | C |
| ATOM | 8524 | O | ALA | C | 330 | −60.666 | −6.948 | 81.356 | 1.00 | 94.65 | O |
| ATOM | 8525 | N | PHE | C | 331 | −58.874 | −6.798 | 82.637 | 1.00 | 80.63 | N |
| ATOM | 8526 | CA | PHE | C | 331 | −59.654 | −6.964 | 83.859 | 1.00 | 84.76 | C |
| ATOM | 8527 | CB | PHE | C | 331 | −58.769 | −7.397 | 85.031 | 1.00 | 87.97 | C |
| ATOM | 8528 | CG | PHE | C | 331 | −58.192 | −8.776 | 84.848 | 1.00 | 87.91 | C |
| ATOM | 8529 | CD2 | PHE | C | 331 | −56.934 | −8.949 | 84.300 | 1.00 | 84.29 | C |
| ATOM | 8530 | CE2 | PHE | C | 331 | −56.413 | −10.217 | 84.109 | 1.00 | 86.27 | C |
| ATOM | 8531 | CZ | PHE | C | 331 | −57.154 | −11.329 | 84.454 | 1.00 | 73.20 | C |
| ATOM | 8532 | CE1 | PHE | C | 331 | −58.415 | −11.173 | 84.988 | 1.00 | 70.54 | C |
| ATOM | 8533 | CD1 | PHE | C | 331 | −58.932 | −9.902 | 85.177 | 1.00 | 83.74 | C |
| ATOM | 8534 | C | PHE | C | 331 | −60.622 | −5.822 | 84.182 | 1.00 | 82.03 | C |
| ATOM | 8535 | O | PHE | C | 331 | −61.687 | −6.030 | 84.711 | 1.00 | 86.49 | O |
| ATOM | 8536 | N | VAL | C | 332 | −60.202 | −4.613 | 83.880 | 1.00 | 82.26 | N |
| ATOM | 8537 | CA | VAL | C | 332 | −61.028 | −3.369 | 83.931 | 1.00 | 87.24 | C |
| ATOM | 8538 | CB | VAL | C | 332 | −60.174 | −2.111 | 84.183 | 1.00 | 87.93 | C |
| ATOM | 8539 | CG2 | VAL | C | 332 | −59.518 | −1.646 | 82.890 | 1.00 | 86.62 | C |
| ATOM | 8540 | CG1 | VAL | C | 332 | −59.123 | −2.385 | 85.247 | 1.00 | 86.03 | C |
| ATOM | 8541 | C | VAL | C | 332 | −61.842 | −3.163 | 82.638 | 1.00 | 83.56 | C |
| ATOM | 8542 | O | VAL | C | 332 | −61.448 | −3.674 | 81.617 | 1.00 | 85.08 | O |
| ATOM | 8543 | N | ASN | C | 333 | −62.895 | −2.327 | 82.622 | 1.00 | 85.50 | N |
| ATOM | 8544 | CA | ASN | C | 333 | −63.498 | −2.019 | 81.305 | 1.00 | 92.85 | C |
| ATOM | 8545 | CB | ASN | C | 333 | −64.585 | −3.034 | 80.895 | 1.00 | 99.30 | C |
| ATOM | 8546 | CG | ASN | C | 333 | −65.775 | −3.076 | 81.853 | 1.00 | 91.27 | C |
| ATOM | 8547 | OD1 | ASN | C | 333 | −66.631 | −3.955 | 81.737 | 1.00 | 73.62 | O |
| ATOM | 8548 | ND2 | ASN | C | 333 | −65.841 | −2.134 | 82.786 | 1.00 | 94.95 | N |
| ATOM | 8549 | C | ASN | C | 333 | −63.981 | −0.557 | 81.199 | 1.00 | 96.08 | C |
| ATOM | 8550 | O | ASN | C | 333 | −64.506 | −0.011 | 82.156 | 1.00 | 98.57 | O |
| ATOM | 8551 | N | PRO | C | 334 | −63.766 | 0.174 | 80.041 | 1.00 | 97.62 | N |
| ATOM | 8552 | CA | PRO | C | 334 | −64.163 | 1.583 | 79.879 | 1.00 | 97.40 | C |
| ATOM | 8553 | CB | PRO | C | 334 | −63.697 | 1.912 | 78.464 | 1.00 | 102.11 | C |
| ATOM | 8554 | CG | PRO | C | 334 | −63.788 | 0.614 | 77.743 | 1.00 | 100.74 | C |
| ATOM | 8555 | CD | PRO | C | 334 | −63.398 | −0.436 | 78.754 | 1.00 | 98.73 | C |
| ATOM | 8556 | C | PRO | C | 334 | −65.688 | 1.655 | 79.970 | 1.00 | 105.23 | C |
| ATOM | 8557 | O | PRO | C | 334 | −66.344 | 0.689 | 79.582 | 1.00 | 108.54 | O |
| ATOM | 8558 | O | GLY | C | 335 | −67.736 | 4.865 | 80.701 | 1.00 | 108.87 | O |
| ATOM | 8559 | N | GLY | C | 335 | −66.248 | 2.697 | 80.587 | 1.00 | 104.43 | N |
| ATOM | 8560 | CA | GLY | C | 335 | −65.578 | 3.900 | 80.992 | 1.00 | 104.97 | C |
| ATOM | 8561 | C | GLY | C | 335 | −66.530 | 5.055 | 80.882 | 1.00 | 114.34 | C |
| ATOM | 8562 | O | GLU | C | 336 | −68.700 | 8.591 | 80.521 | 1.00 | 110.76 | O |
| ATOM | 8563 | N | GLU | C | 336 | −66.022 | 6.220 | 81.294 | 1.00 | 124.19 | N |
| ATOM | 8564 | CA | GLU | C | 336 | −66.869 | 7.391 | 81.553 | 1.00 | 126.45 | C |
| ATOM | 8565 | C | GLU | C | 336 | −67.766 | 7.814 | 80.359 | 1.00 | 114.03 | C |
| ATOM | 8566 | CB | GLU | C | 336 | −66.004 | 8.577 | 81.992 | 1.00 | 131.50 | C |
| ATOM | 8567 | CG | GLU | C | 336 | −64.980 | 8.236 | 83.067 | 1.00 | 130.36 | C |
| ATOM | 8568 | CD | GLU | C | 336 | −64.107 | 9.417 | 83.441 | 1.00 | 132.35 | C |
| ATOM | 8569 | OE1 | GLU | C | 336 | −64.451 | 10.555 | 83.055 | 1.00 | 134.34 | O |
| ATOM | 8570 | OE2 | GLU | C | 336 | −63.077 | 9.207 | 84.119 | 1.00 | 119.74 | O |
| ATOM | 8571 | O | ASP | C | 337 | −68.594 | 9.717 | 76.771 | 1.00 | 115.70 | O |
| ATOM | 8572 | N | ASP | C | 337 | −67.437 | 7.305 | 79.170 | 1.00 | 114.18 | N |
| ATOM | 8573 | CA | ASP | C | 337 | −68.126 | 7.645 | 77.938 | 1.00 | 117.80 | C |
| ATOM | 8574 | C | ASP | C | 337 | −67.869 | 9.096 | 77.548 | 1.00 | 112.46 | C |
| ATOM | 8575 | CB | ASP | C | 337 | −69.625 | 7.329 | 78.019 | 1.00 | 117.86 | C |
| ATOM | 8576 | CG | ASP | C | 337 | −69.888 | 5.863 | 78.293 | 1.00 | 116.12 | C |
| ATOM | 8577 | OD1 | ASP | C | 337 | −69.327 | 5.012 | 77.565 | 1.00 | 114.81 | O |
| ATOM | 8578 | OD2 | ASP | C | 337 | −70.645 | 5.557 | 79.237 | 1.00 | 118.19 | O |
| ATOM | 8579 | N | GLU | C | 338 | −66.646 | 9.476 | 77.868 | 1.00 | 105.64 | N |
| ATOM | 8580 | CA | GLU | C | 338 | −65.949 | 10.515 | 77.143 | 1.00 | 115.01 | C |
| ATOM | 8581 | C | GLU | C | 338 | −65.506 | 9.938 | 75.797 | 1.00 | 113.73 | C |
| ATOM | 8582 | O | GLU | C | 338 | −65.664 | 8.754 | 75.506 | 1.00 | 105.34 | O |
| ATOM | 8583 | CB | GLU | C | 338 | −64.648 | 10.946 | 77.843 | 1.00 | 121.33 | C |
| ATOM | 8584 | CG | GLU | C | 338 | −64.546 | 10.958 | 79.354 | 1.00 | 122.59 | C |
| ATOM | 8585 | CD | GLU | C | 338 | −63.257 | 11.631 | 79.787 | 1.00 | 119.55 | C |
| ATOM | 8586 | OE1 | GLU | C | 338 | −62.429 | 11.978 | 78.893 | 1.00 | 103.48 | O |
| ATOM | 8587 | OE2 | GLU | C | 338 | −63.091 | 11.822 | 81.014 | 1.00 | 124.10 | O |
| ATOM | 8588 | N | VAL | C | 339 | −64.782 | 10.773 | 75.075 | 1.00 | 115.80 | N |
| ATOM | 8589 | CA | VAL | C | 339 | −63.881 | 10.282 | 74.052 | 1.00 | 115.02 | C |
| ATOM | 8590 | CB | VAL | C | 339 | −63.136 | 11.444 | 73.358 | 1.00 | 116.42 | C |
| ATOM | 8591 | CG1 | VAL | C | 339 | −62.505 | 10.983 | 72.047 | 1.00 | 109.73 | C |
| ATOM | 8592 | CG2 | VAL | C | 339 | −64.093 | 12.598 | 73.100 | 1.00 | 113.32 | C |
| ATOM | 8593 | C | VAL | C | 339 | −62.855 | 9.266 | 74.657 | 1.00 | 110.96 | C |
| ATOM | 8594 | O | VAL | C | 339 | −62.186 | 8.570 | 73.933 | 1.00 | 111.21 | O |
| ATOM | 8595 | N | SER | C | 340 | −62.834 | 9.118 | 75.998 | 1.00 | 110.20 | N |
| ATOM | 8596 | CA | SER | C | 340 | −62.046 | 8.100 | 76.698 | 1.00 | 106.00 | C |
| ATOM | 8597 | CB | SER | C | 340 | −62.059 | 8.323 | 78.213 | 1.00 | 107.69 | C |
| ATOM | 8598 | OG | SER | C | 340 | −63.295 | 7.921 | 78.778 | 1.00 | 112.38 | O |
| ATOM | 8599 | C | SER | C | 340 | −62.484 | 6.678 | 76.371 | 1.00 | 102.85 | C |
| ATOM | 8600 | O | SER | C | 340 | −61.666 | 5.768 | 76.336 | 1.00 | 95.74 | O |
| ATOM | 8601 | N | ARG | C | 341 | −63.773 | 6.483 | 76.085 | 1.00 | 109.32 | N |
| ATOM | 8602 | CA | ARG | C | 341 | −64.211 | 5.178 | 75.592 | 1.00 | 107.15 | C |
| ATOM | 8603 | CB | ARG | C | 341 | −65.743 | 5.142 | 75.517 | 1.00 | 107.16 | C |
| ATOM | 8604 | CG | ARG | C | 341 | −66.324 | 4.209 | 74.463 | 1.00 | 97.86 | C |
| ATOM | 8605 | CD | ARG | C | 341 | −67.853 | 4.155 | 74.539 | 1.00 | 106.91 | C |
| ATOM | 8606 | NE | ARG | C | 341 | −68.466 | 5.485 | 74.547 | 1.00 | 123.18 | N |
| ATOM | 8607 | CZ | ARG | C | 341 | −69.776 | 5.713 | 74.472 | 1.00 | 110.97 | C |
| ATOM | 8608 | NH1 | ARG | C | 341 | −70.625 | 4.699 | 74.375 | 1.00 | 105.47 | N |
| ATOM | 8609 | NH2 | ARG | C | 341 | −70.240 | 6.957 | 74.491 | 1.00 | 97.14 | N |
| ATOM | 8610 | C | ARG | C | 341 | −63.584 | 4.871 | 74.208 | 1.00 | 98.03 | C |
| ATOM | 8611 | O | ARG | C | 341 | −63.104 | 3.768 | 73.972 | 1.00 | 91.88 | O |
| ATOM | 8612 | N | VAL | C | 342 | −63.547 | 5.893 | 73.327 | 1.00 | 96.06 | N |
| ATOM | 8613 | CA | VAL | C | 342 | −62.805 | 5.838 | 72.062 | 1.00 | 93.36 | C |
| ATOM | 8614 | CB | VAL | C | 342 | −62.959 | 7.150 | 71.266 | 1.00 | 99.64 | C |
| ATOM | 8615 | CG1 | VAL | C | 342 | −62.379 | 6.997 | 69.865 | 1.00 | 100.81 | C |
| ATOM | 8616 | CG2 | VAL | C | 342 | −64.422 | 7.562 | 71.206 | 1.00 | 107.65 | C |
| ATOM | 8617 | C | VAL | C | 342 | −61.308 | 5.517 | 72.215 | 1.00 | 91.66 | C |
| ATOM | 8618 | O | VAL | C | 342 | −60.792 | 4.577 | 71.632 | 1.00 | 89.72 | O |
| ATOM | 8619 | N | VAL | C | 343 | −60.612 | 6.340 | 73.008 | 1.00 | 93.62 | N |
| ATOM | 8620 | CA | VAL | C | 343 | −59.185 | 6.218 | 73.246 | 1.00 | 88.17 | C |
| ATOM | 8621 | CB | VAL | C | 343 | −58.670 | 7.309 | 74.203 | 1.00 | 86.99 | C |
| ATOM | 8622 | CG1 | VAL | C | 343 | −57.184 | 7.121 | 74.472 | 1.00 | 85.29 | C |
| ATOM | 8623 | CG2 | VAL | C | 343 | −58.932 | 8.685 | 73.620 | 1.00 | 89.18 | C |
| ATOM | 8624 | C | VAL | C | 343 | −58.836 | 4.844 | 73.792 | 1.00 | 85.35 | C |
| ATOM | 8625 | O | VAL | C | 343 | −57.921 | 4.208 | 73.328 | 1.00 | 84.18 | O |
| ATOM | 8626 | N | PHE | C | 344 | −59.620 | 4.361 | 74.743 | 1.00 | 82.03 | N |
| ATOM | 8627 | CA | PHE | C | 344 | −59.421 | 3.035 | 75.266 | 1.00 | 74.14 | C |
| ATOM | 8628 | CB | PHE | C | 344 | −60.485 | 2.740 | 76.326 | 1.00 | 78.21 | C |
| ATOM | 8629 | CG | PHE | C | 344 | −60.291 | 1.435 | 77.059 | 1.00 | 85.80 | C |
| ATOM | 8630 | CD2 | PHE | C | 344 | −59.872 | 1.430 | 78.381 | 1.00 | 95.27 | C |
| ATOM | 8631 | CE2 | PHE | C | 344 | −59.716 | 0.240 | 79.071 | 1.00 | 94.37 | C |
| ATOM | 8632 | CZ | PHE | C | 344 | −59.986 | −0.962 | 78.443 | 1.00 | 92.83 | C |
| ATOM | 8633 | CE1 | PHE | C | 344 | −60.416 | −0.971 | 77.124 | 1.00 | 92.68 | C |
| ATOM | 8634 | CD1 | PHE | C | 344 | −60.578 | 0.220 | 76.448 | 1.00 | 85.69 | C |
| ATOM | 8635 | C | PHE | C | 344 | −59.504 | 1.980 | 74.174 | 1.00 | 78.41 | C |
| ATOM | 8636 | O | PHE | C | 344 | −58.714 | 1.065 | 74.134 | 1.00 | 76.70 | O |
| ATOM | 8637 | N | ILE | C | 345 | −60.537 | 2.059 | 73.346 | 1.00 | 87.88 | N |
| ATOM | 8638 | CA | ILE | C | 345 | −60.738 | 1.046 | 72.323 | 1.00 | 86.78 | C |
| ATOM | 8639 | CB | ILE | C | 345 | −62.107 | 1.227 | 71.629 | 1.00 | 75.61 | C |
| ATOM | 8640 | CG1 | ILE | C | 345 | −63.238 | 0.899 | 72.604 | 1.00 | 81.27 | C |
| ATOM | 8641 | CD1 | ILE | C | 345 | −64.614 | 1.284 | 72.094 | 1.00 | 87.34 | C |
| ATOM | 8642 | CG2 | ILE | C | 345 | −62.214 | 0.354 | 70.386 | 1.00 | 64.09 | C |
| ATOM | 8643 | C | ILE | C | 345 | −59.626 | 1.041 | 71.244 | 1.00 | 81.16 | C |
| ATOM | 8644 | O | ILE | C | 345 | −59.107 | −0.013 | 70.893 | 1.00 | 78.97 | O |
| ATOM | 8645 | N | TYR | C | 346 | −59.187 | 2.233 | 70.814 | 1.00 | 78.00 | N |
| ATOM | 8646 | CA | TYR | C | 346 | −58.038 | 2.386 | 69.912 | 1.00 | 75.48 | C |
| ATOM | 8647 | CB | TYR | C | 346 | −57.879 | 3.855 | 69.511 | 1.00 | 74.76 | C |
| ATOM | 8648 | CG | TYR | C | 346 | −58.683 | 4.269 | 68.301 | 1.00 | 81.55 | C |
| ATOM | 8649 | CD2 | TYR | C | 346 | −58.065 | 4.488 | 67.073 | 1.00 | 79.83 | C |
| ATOM | 8650 | CE2 | TYR | C | 346 | −58.794 | 4.878 | 65.965 | 1.00 | 88.49 | C |
| ATOM | 8651 | CZ | TYR | C | 346 | −60.157 | 5.058 | 66.083 | 1.00 | 99.96 | C |
| ATOM | 8652 | OH | TYR | C | 346 | −60.902 | 5.447 | 64.994 | 1.00 | 108.77 | O |
| ATOM | 8653 | CE1 | TYR | C | 346 | −60.789 | 4.852 | 67.292 | 1.00 | 89.67 | C |
| ATOM | 8654 | CD1 | TYR | C | 346 | −60.054 | 4.465 | 68.390 | 1.00 | 89.61 | C |
| ATOM | 8655 | C | TYR | C | 346 | −56.723 | 1.877 | 70.527 | 1.00 | 75.34 | C |
| ATOM | 8656 | O | TYR | C | 346 | −55.925 | 1.206 | 69.906 | 1.00 | 72.71 | O |
| ATOM | 8657 | N | PHE | C | 347 | −56.493 | 2.304 | 71.753 | 1.00 | 76.71 | N |
| ATOM | 8658 | CA | PHE | C | 347 | −55.266 | 2.063 | 72.484 | 1.00 | 71.10 | C |
| ATOM | 8659 | CB | PHE | C | 347 | −55.261 | 2.979 | 73.711 | 1.00 | 74.58 | C |
| ATOM | 8660 | CG | PHE | C | 347 | −53.999 | 2.954 | 74.512 | 1.00 | 75.81 | C |
| ATOM | 8661 | CD1 | PHE | C | 347 | −52.818 | 3.453 | 73.989 | 1.00 | 70.80 | C |
| ATOM | 8662 | CE1 | PHE | C | 347 | −51.663 | 3.453 | 74.742 | 1.00 | 78.12 | C |
| ATOM | 8663 | CZ | PHE | C | 347 | −51.685 | 2.971 | 76.042 | 1.00 | 84.23 | C |
| ATOM | 8664 | CE2 | PHE | C | 347 | −52.863 | 2.489 | 76.579 | 1.00 | 76.07 | C |
| ATOM | 8665 | CD2 | PHE | C | 347 | −54.011 | 2.490 | 75.819 | 1.00 | 78.27 | C |
| ATOM | 8666 | C | PHE | C | 347 | −55.125 | 0.585 | 72.910 | 1.00 | 71.68 | C |
| ATOM | 8667 | O | PHE | C | 347 | −54.077 | −0.011 | 72.772 | 1.00 | 66.83 | O |
| ATOM | 8668 | N | ASN | C | 348 | −56.236 | −0.036 | 73.317 | 1.00 | 76.65 | N |
| ATOM | 8669 | CA | ASN | C | 348 | −56.266 | −1.475 | 73.578 | 1.00 | 74.68 | C |
| ATOM | 8670 | CB | ASN | C | 348 | −57.619 | −1.929 | 74.127 | 1.00 | 72.00 | C |
| ATOM | 8671 | CG | ASN | C | 348 | −57.548 | −3.306 | 74.745 | 1.00 | 69.31 | C |
| ATOM | 8672 | OD1 | ASN | C | 348 | −57.056 | −3.463 | 75.861 | 1.00 | 71.41 | O |
| ATOM | 8673 | ND2 | ASN | C | 348 | −58.005 | −4.318 | 74.012 | 1.00 | 70.62 | N |
| ATOM | 8674 | C | ASN | C | 348 | −55.899 | −2.280 | 72.336 | 1.00 | 74.07 | C |
| ATOM | 8675 | O | ASN | C | 348 | −55.147 | −3.222 | 72.407 | 1.00 | 76.17 | O |
| ATOM | 8676 | N | ALA | C | 349 | −56.463 | −1.911 | 71.184 | 1.00 | 75.62 | N |
| ATOM | 8677 | CA | ALA | C | 349 | −56.284 | −2.694 | 69.980 | 1.00 | 69.32 | C |
| ATOM | 8678 | CB | ALA | C | 349 | −57.185 | −2.174 | 68.871 | 1.00 | 69.01 | C |
| ATOM | 8679 | C | ALA | C | 349 | −54.841 | −2.669 | 69.539 | 1.00 | 69.57 | C |
| ATOM | 8680 | O | ALA | C | 349 | −54.261 | −3.692 | 69.276 | 1.00 | 73.00 | O |
| ATOM | 8681 | N | PHE | C | 350 | −54.217 | −1.516 | 69.633 | 1.00 | 67.10 | N |
| ATOM | 8682 | CA | PHE | C | 350 | −52.835 | −1.378 | 69.243 | 1.00 | 67.23 | C |
| ATOM | 8683 | CB | PHE | C | 350 | −52.461 | 0.106 | 69.251 | 1.00 | 64.49 | C |
| ATOM | 8684 | CG | PHE | C | 350 | −51.012 | 0.380 | 68.978 | 1.00 | 69.61 | C |
| ATOM | 8685 | CD1 | PHE | C | 350 | −50.532 | 0.415 | 67.679 | 1.00 | 73.41 | C |
| ATOM | 8686 | CE1 | PHE | C | 350 | −49.198 | 0.683 | 67.427 | 1.00 | 78.40 | C |
| ATOM | 8687 | CZ | PHE | C | 350 | −48.330 | 0.930 | 68.478 | 1.00 | 76.77 | C |
| ATOM | 8688 | CE2 | PHE | C | 350 | −48.799 | 0.907 | 69.776 | 1.00 | 73.36 | C |
| ATOM | 8689 | CD2 | PHE | C | 350 | −50.134 | 0.638 | 70.021 | 1.00 | 71.24 | C |
| ATOM | 8690 | C | PHE | C | 350 | −51.877 | −2.189 | 70.144 | 1.00 | 65.72 | C |
| ATOM | 8691 | O | PHE | C | 350 | −50.924 | −2.774 | 69.680 | 1.00 | 66.24 | O |
| ATOM | 8692 | N | LEU | C | 351 | −52.073 | −2.138 | 71.455 | 1.00 | 65.93 | N |
| ATOM | 8693 | CA | LEU | C | 351 | −51.131 | −2.769 | 72.370 | 1.00 | 59.60 | C |
| ATOM | 8694 | CB | LEU | C | 351 | −51.349 | −2.265 | 73.793 | 1.00 | 60.62 | C |
| ATOM | 8695 | CG | LEU | C | 351 | −51.030 | −0.791 | 74.023 | 1.00 | 67.96 | C |
| ATOM | 8696 | CD1 | LEU | C | 351 | −51.347 | −0.429 | 75.457 | 1.00 | 75.62 | C |
| ATOM | 8697 | CD2 | LEU | C | 351 | −49.575 | −0.494 | 73.694 | 1.00 | 58.15 | C |
| ATOM | 8698 | C | LEU | C | 351 | −51.183 | −4.277 | 72.357 | 1.00 | 66.19 | C |
| ATOM | 8699 | O | LEU | C | 351 | −50.161 | −4.928 | 72.281 | 1.00 | 70.02 | O |
| ATOM | 8700 | N | GLU | C | 352 | −52.384 | −4.840 | 72.393 | 1.00 | 63.32 | N |
| ATOM | 8701 | CA | GLU | C | 352 | −52.540 | −6.273 | 72.293 | 1.00 | 61.62 | C |
| ATOM | 8702 | CB | GLU | C | 352 | −53.932 | −6.771 | 72.696 | 1.00 | 64.21 | C |
| ATOM | 8703 | CG | GLU | C | 352 | −55.047 | −6.504 | 71.730 | 1.00 | 71.47 | C |
| ATOM | 8704 | CD | GLU | C | 352 | −56.304 | −7.242 | 72.132 | 1.00 | 85.48 | C |
| ATOM | 8705 | OE1 | GLU | C | 352 | −57.227 | −6.597 | 72.675 | 1.00 | 84.94 | O |
| ATOM | 8706 | OE2 | GLU | C | 352 | −56.358 | −8.473 | 71.920 | 1.00 | 81.33 | O |
| ATOM | 8707 | C | GLU | C | 352 | −52.060 | −6.834 | 70.966 | 1.00 | 59.17 | C |
| ATOM | 8708 | O | GLU | C | 352 | −51.508 | −7.920 | 70.897 | 1.00 | 61.41 | O |
| ATOM | 8709 | N | SER | C | 353 | −52.411 | −6.129 | 69.891 | 1.00 | 54.89 | N |
| ATOM | 8710 | CA | SER | C | 353 | −52.104 | −6.569 | 68.561 | 1.00 | 53.92 | C |
| ATOM | 8711 | CB | SER | C | 353 | −52.907 | −5.754 | 67.551 | 1.00 | 58.03 | C |
| ATOM | 8712 | OG | SER | C | 353 | −52.830 | −6.325 | 66.262 | 1.00 | 64.77 | O |
| ATOM | 8713 | C | SER | C | 353 | −50.638 | −6.521 | 68.188 | 1.00 | 54.97 | C |
| ATOM | 8714 | O | SER | C | 353 | −50.203 | −7.185 | 67.266 | 1.00 | 53.93 | O |
| ATOM | 8715 | N | PHE | C | 354 | −49.888 | −5.737 | 68.923 | 1.00 | 56.68 | N |
| ATOM | 8716 | CA | PHE | C | 354 | −48.477 | −5.534 | 68.679 | 1.00 | 52.74 | C |
| ATOM | 8717 | CB | PHE | C | 354 | −48.136 | −4.044 | 68.596 | 1.00 | 53.55 | C |
| ATOM | 8718 | CG | PHE | C | 354 | −48.368 | −3.448 | 67.234 | 1.00 | 59.14 | C |
| ATOM | 8719 | CD2 | PHE | C | 354 | −47.348 | −3.411 | 66.299 | 1.00 | 57.72 | C |
| ATOM | 8720 | CE2 | PHE | C | 354 | −47.555 | −2.868 | 65.042 | 1.00 | 56.83 | C |
| ATOM | 8721 | CZ | PHE | C | 354 | −48.791 | −2.354 | 64.708 | 1.00 | 53.31 | C |
| ATOM | 8722 | CE1 | PHE | C | 354 | −49.818 | −2.384 | 65.630 | 1.00 | 57.68 | C |
| ATOM | 8723 | CD1 | PHE | C | 354 | −49.606 | −2.931 | 66.885 | 1.00 | 62.56 | C |
| ATOM | 8724 | C | PHE | C | 354 | −47.617 | −6.248 | 69.709 | 1.00 | 52.42 | C |
| ATOM | 8725 | O | PHE | C | 354 | −46.430 | −6.033 | 69.772 | 1.00 | 52.75 | O |
| ATOM | 8726 | N | GLN | C | 355 | −48.223 | −7.100 | 70.533 | 1.00 | 54.35 | N |
| ATOM | 8727 | CA | GLN | C | 355 | −47.481 | −7.876 | 71.503 | 1.00 | 51.54 | C |
| ATOM | 8728 | CB | GLN | C | 355 | −48.391 | −8.837 | 72.262 | 1.00 | 50.24 | C |
| ATOM | 8729 | CG | GLN | C | 355 | −48.895 | −8.283 | 73.573 | 1.00 | 57.47 | C |
| ATOM | 8730 | CD | GLN | C | 355 | −49.070 | −9.363 | 74.617 | 1.00 | 66.71 | C |
| ATOM | 8731 | OE1 | GLN | C | 355 | −48.464 | −10.434 | 74.529 | 1.00 | 59.98 | O |
| ATOM | 8732 | NE2 | GLN | C | 355 | −49.905 | −9.092 | 75.614 | 1.00 | 70.83 | N |
| ATOM | 8733 | C | GLN | C | 355 | −46.365 | −8.640 | 70.851 | 1.00 | 52.73 | C |
| ATOM | 8734 | O | GLN | C | 355 | −45.290 | −8.735 | 71.382 | 1.00 | 54.70 | O |
| ATOM | 8735 | N | GLY | C | 356 | −46.636 | −9.209 | 69.700 | 1.00 | 54.12 | N |
| ATOM | 8736 | CA | GLY | C | 356 | −45.655 | −9.977 | 68.978 | 1.00 | 48.65 | C |
| ATOM | 8737 | C | GLY | C | 356 | −44.479 | −9.206 | 68.446 | 1.00 | 50.89 | C |
| ATOM | 8738 | O | GLY | C | 356 | −43.347 | −9.622 | 68.594 | 1.00 | 48.79 | O |
| ATOM | 8739 | N | PHE | C | 357 | −44.747 | −8.038 | 67.874 | 1.00 | 50.46 | N |
| ATOM | 8740 | CA | PHE | C | 357 | −43.701 | −7.138 | 67.420 | 1.00 | 53.83 | C |
| ATOM | 8741 | CB | PHE | C | 357 | −44.269 | −5.893 | 66.737 | 1.00 | 48.51 | C |
| ATOM | 8742 | CG | PHE | C | 357 | −43.210 | −5.008 | 66.147 | 1.00 | 55.96 | C |
| ATOM | 8743 | CD1 | PHE | C | 357 | −42.629 | −5.323 | 64.933 | 1.00 | 59.24 | C |
| ATOM | 8744 | CE1 | PHE | C | 357 | −41.643 | −4.518 | 64.393 | 1.00 | 60.92 | C |
| ATOM | 8745 | CZ | PHE | C | 357 | −41.225 | −3.387 | 65.067 | 1.00 | 51.88 | C |
| ATOM | 8746 | CE2 | PHE | C | 357 | −41.791 | −3.065 | 66.278 | 1.00 | 45.12 | C |
| ATOM | 8747 | CD2 | PHE | C | 357 | −42.776 | −3.875 | 66.816 | 1.00 | 56.74 | C |
| ATOM | 8748 | C | PHE | C | 357 | −42.778 | −6.748 | 68.580 | 1.00 | 55.38 | C |
| ATOM | 8749 | O | PHE | C | 357 | −41.568 | −6.753 | 68.466 | 1.00 | 56.90 | O |
| ATOM | 8750 | N | PHE | C | 358 | −43.365 | −6.465 | 69.727 | 1.00 | 50.33 | N |
| ATOM | 8751 | CA | PHE | C | 358 | −42.573 | −6.124 | 70.874 | 1.00 | 46.37 | C |
| ATOM | 8752 | CB | PHE | C | 358 | −43.461 | −5.744 | 72.065 | 1.00 | 43.57 | C |
| ATOM | 8753 | CG | PHE | C | 358 | −44.379 | −4.586 | 71.805 | 1.00 | 47.10 | C |
| ATOM | 8754 | CD1 | PHE | C | 358 | −44.159 | −3.724 | 70.741 | 1.00 | 50.55 | C |
| ATOM | 8755 | CE1 | PHE | C | 358 | −45.011 | −2.659 | 70.506 | 1.00 | 52.00 | C |
| ATOM | 8756 | CZ | PHE | C | 358 | −46.092 | −2.440 | 71.342 | 1.00 | 52.32 | C |
| ATOM | 8757 | CE2 | PHE | C | 358 | −46.319 | −3.290 | 72.411 | 1.00 | 52.12 | C |
| ATOM | 8758 | CD2 | PHE | C | 358 | −45.463 | −4.354 | 72.637 | 1.00 | 49.15 | C |
| ATOM | 8759 | C | PHE | C | 358 | −41.679 | −7.276 | 71.309 | 1.00 | 55.36 | C |
| ATOM | 8760 | O | PHE | C | 358 | −40.497 | −7.111 | 71.532 | 1.00 | 64.14 | O |
| ATOM | 8761 | N | VAL | C | 359 | −42.229 | −8.464 | 71.374 | 1.00 | 55.34 | N |
| ATOM | 8762 | CA | VAL | C | 359 | −41.465 | −9.656 | 71.711 | 1.00 | 60.00 | C |
| ATOM | 8763 | CB | VAL | C | 359 | −42.375 | −10.902 | 71.882 | 1.00 | 50.47 | C |
| ATOM | 8764 | CG1 | VAL | C | 359 | −41.551 | −12.142 | 72.206 | 1.00 | 44.21 | C |
| ATOM | 8765 | CG2 | VAL | C | 359 | −43.382 | −10.657 | 72.985 | 1.00 | 48.13 | C |
| ATOM | 8766 | C | VAL | C | 359 | −40.319 | −9.931 | 70.732 | 1.00 | 61.93 | C |
| ATOM | 8767 | O | VAL | C | 359 | −39.220 | −10.282 | 71.136 | 1.00 | 62.57 | O |
| ATOM | 8768 | N | SER | C | 360 | −40.559 | −9.745 | 69.438 | 1.00 | 53.81 | N |
| ATOM | 8769 | CA | SER | C | 360 | −39.502 | −9.934 | 68.459 | 1.00 | 55.15 | C |
| ATOM | 8770 | CB | SER | C | 360 | −40.035 | −9.720 | 67.044 | 1.00 | 46.65 | C |
| ATOM | 8771 | OG | SER | C | 360 | −40.419 | −8.371 | 66.855 | 1.00 | 55.28 | O |
| ATOM | 8772 | C | SER | C | 360 | −38.320 | −8.988 | 68.709 | 1.00 | 66.79 | C |
| ATOM | 8773 | O | SER | C | 360 | −37.173 | −9.404 | 68.719 | 1.00 | 70.20 | O |
| ATOM | 8774 | N | VAL | C | 361 | −38.623 | −7.713 | 68.985 | 1.00 | 64.19 | N |
| ATOM | 8775 | CA | VAL | C | 361 | −37.593 | −6.742 | 69.338 | 1.00 | 61.45 | C |
| ATOM | 8776 | CB | VAL | C | 361 | −38.200 | −5.344 | 69.587 | 1.00 | 60.67 | C |
| ATOM | 8777 | CG1 | VAL | C | 361 | −37.147 | −4.389 | 70.135 | 1.00 | 55.85 | C |
| ATOM | 8778 | CG2 | VAL | C | 361 | −38.816 | −4.798 | 68.305 | 1.00 | 61.97 | C |
| ATOM | 8779 | C | VAL | C | 361 | −36.796 | −7.157 | 70.560 | 1.00 | 59.65 | C |
| ATOM | 8780 | O | VAL | C | 361 | −35.597 | −7.077 | 70.569 | 1.00 | 60.17 | O |
| ATOM | 8781 | N | PHE | C | 362 | −37.472 | −7.612 | 71.583 | 1.00 | 61.92 | N |
| ATOM | 8782 | CA | PHE | C | 362 | −36.816 | −8.016 | 72.813 | 1.00 | 63.14 | C |
| ATOM | 8783 | CB | PHE | C | 362 | −37.928 | −8.309 | 73.841 | 1.00 | 68.27 | C |
| ATOM | 8784 | CG | PHE | C | 362 | −37.532 | −9.210 | 74.980 | 1.00 | 66.21 | C |
| ATOM | 8785 | CD1 | PHE | C | 362 | −37.038 | −8.683 | 76.161 | 1.00 | 64.90 | C |
| ATOM | 8786 | CE1 | PHE | C | 362 | −36.703 | −9.508 | 77.220 | 1.00 | 64.40 | C |
| ATOM | 8787 | CZ | PHE | C | 362 | −36.882 | −10.873 | 77.114 | 1.00 | 57.93 | C |
| ATOM | 8788 | CE2 | PHE | C | 362 | −37.394 | −11.410 | 75.951 | 1.00 | 63.83 | C |
| ATOM | 8789 | CD2 | PHE | C | 362 | −37.729 | −10.580 | 74.897 | 1.00 | 65.59 | C |
| ATOM | 8790 | C | PHE | C | 362 | −35.843 | −9.221 | 72.631 | 1.00 | 65.39 | C |
| ATOM | 8791 | O | PHE | C | 362 | −34.706 | −9.209 | 73.103 | 1.00 | 72.35 | O |
| ATOM | 8792 | N | ALA | C | 363 | −36.273 | −10.259 | 71.903 | 1.00 | 67.56 | N |
| ATOM | 8793 | CA | ALA | C | 363 | −35.379 | −11.393 | 71.609 | 1.00 | 67.62 | C |
| ATOM | 8794 | CB | ALA | C | 363 | −36.150 | −12.504 | 70.919 | 1.00 | 72.41 | C |
| ATOM | 8795 | C | ALA | C | 363 | −34.167 | −10.987 | 70.778 | 1.00 | 67.72 | C |
| ATOM | 8796 | O | ALA | C | 363 | −33.045 | −11.341 | 71.083 | 1.00 | 72.65 | O |
| ATOM | 8797 | N | CYS | C | 364 | −34.431 | −10.158 | 69.769 | 1.00 | 67.75 | N |
| ATOM | 8798 | CA | CYS | C | 364 | −33.431 | −9.562 | 68.886 | 1.00 | 66.54 | C |
| ATOM | 8799 | CB | CYS | C | 364 | −34.091 | −8.762 | 67.760 | 1.00 | 69.39 | C |
| ATOM | 8800 | SG | CYS | C | 364 | −34.847 | −9.795 | 66.473 | 1.00 | 76.93 | S |
| ATOM | 8801 | C | CYS | C | 364 | −32.454 | −8.700 | 69.664 | 1.00 | 68.38 | C |
| ATOM | 8802 | O | CYS | C | 364 | −31.276 | −8.647 | 69.354 | 1.00 | 74.59 | O |
| ATOM | 8803 | N | PHE | C | 365 | −32.962 | −8.020 | 70.687 | 1.00 | 70.17 | N |
| ATOM | 8804 | CA | PHE | C | 365 | −32.135 | −7.154 | 71.518 | 1.00 | 68.83 | C |
| ATOM | 8805 | CB | PHE | C | 365 | −32.999 | −6.299 | 72.449 | 1.00 | 69.64 | C |
| ATOM | 8806 | CG | PHE | C | 365 | −32.226 | −5.251 | 73.198 | 1.00 | 74.54 | C |
| ATOM | 8807 | CD1 | PHE | C | 365 | −31.511 | −4.282 | 72.514 | 1.00 | 74.12 | C |
| ATOM | 8808 | CE1 | PHE | C | 365 | −30.800 | −3.314 | 73.197 | 1.00 | 84.31 | C |
| ATOM | 8809 | CZ | PHE | C | 365 | −30.802 | −3.303 | 74.579 | 1.00 | 86.51 | C |
| ATOM | 8810 | CE2 | PHE | C | 365 | −31.515 | −4.261 | 75.273 | 1.00 | 80.97 | C |
| ATOM | 8811 | CD2 | PHE | C | 365 | −32.223 | −5.228 | 74.584 | 1.00 | 79.54 | C |
| ATOM | 8812 | C | PHE | C | 365 | −31.122 | −7.964 | 72.321 | 1.00 | 73.32 | C |
| ATOM | 8813 | O | PHE | C | 365 | −30.006 | −7.508 | 72.569 | 1.00 | 84.10 | O |
| ATOM | 8814 | N | LEU | C | 366 | −31.519 | −9.167 | 72.724 | 1.00 | 71.84 | N |
| ATOM | 8815 | CA | LEU | C | 366 | −30.647 | −10.041 | 73.499 | 1.00 | 76.13 | C |
| ATOM | 8816 | CB | LEU | C | 366 | −31.457 | −11.070 | 74.290 | 1.00 | 75.59 | C |
| ATOM | 8817 | CG | LEU | C | 366 | −32.387 | −10.478 | 75.354 | 1.00 | 68.06 | C |
| ATOM | 8818 | CD1 | LEU | C | 366 | −32.805 | −11.538 | 76.364 | 1.00 | 72.01 | C |
| ATOM | 8819 | CD2 | LEU | C | 366 | −31.737 | −9.292 | 76.056 | 1.00 | 74.85 | C |
| ATOM | 8820 | C | LEU | C | 366 | −29.602 | −10.707 | 72.610 | 1.00 | 79.44 | C |
| ATOM | 8821 | O | LEU | C | 366 | −28.430 | −10.798 | 72.975 | 1.00 | 94.72 | O |
| ATOM | 8822 | O | ASN | C | 367 | −26.915 | −10.954 | 70.732 | 1.00 | 81.57 | O |
| ATOM | 8823 | N | ASN | C | 367 | −30.035 | −11.171 | 71.443 | 1.00 | 74.29 | N |
| ATOM | 8824 | CA | ASN | C | 367 | −29.139 | −11.830 | 70.500 | 1.00 | 79.64 | C |
| ATOM | 8825 | C | ASN | C | 367 | −27.979 | −10.918 | 70.113 | 1.00 | 81.30 | C |
| ATOM | 8826 | CB | ASN | C | 367 | −29.899 | −12.271 | 69.254 | 1.00 | 88.14 | C |
| ATOM | 8827 | CG | ASN | C | 367 | −29.063 | −13.158 | 68.350 | 1.00 | 94.78 | C |
| ATOM | 8828 | OD1 | ASN | C | 367 | −28.108 | −13.805 | 68.803 | 1.00 | 96.75 | O |
| ATOM | 8829 | ND2 | ASN | C | 367 | −29.416 | −13.194 | 67.061 | 1.00 | 93.71 | N |
| ATOM | 8830 | C24 | CP3 | C | 900 | −46.331 | −12.568 | 78.055 | 1.00 | 50.39 | C |
| ATOM | 8831 | C23 | CP3 | C | 900 | −47.279 | −11.762 | 78.910 | 1.00 | 56.17 | C |
| ATOM | 8832 | C20 | CP3 | C | 900 | −46.625 | −11.398 | 80.223 | 1.00 | 62.40 | C |
| ATOM | 8833 | C21 | CP3 | C | 900 | −47.350 | −10.212 | 80.821 | 1.00 | 48.98 | C |
| ATOM | 8834 | C22 | CP3 | C | 900 | −46.515 | −9.588 | 81.908 | 1.00 | 53.29 | C |
| ATOM | 8835 | N19 | CP3 | C | 900 | −45.242 | −11.081 | 79.972 | 1.00 | 68.66 | N |
| ATOM | 8836 | C15 | CP3 | C | 900 | −44.280 | −11.531 | 80.796 | 1.00 | 51.57 | C |
| ATOM | 8837 | C14 | CP3 | C | 900 | −44.422 | −12.696 | 81.522 | 1.00 | 51.37 | C |
| ATOM | 8838 | C13 | CP3 | C | 900 | −43.417 | −13.133 | 82.361 | 1.00 | 52.75 | C |
| ATOM | 8839 | C18 | CP3 | C | 900 | −43.617 | −14.401 | 83.126 | 1.00 | 50.23 | C |
| ATOM | 8840 | N12 | CP3 | C | 900 | −42.266 | −12.467 | 82.515 | 1.00 | 53.89 | N |
| ATOM | 8841 | C16 | CP3 | C | 900 | −43.032 | −10.785 | 80.946 | 1.00 | 54.22 | C |
| ATOM | 8842 | C17 | CP3 | C | 900 | −42.801 | −9.509 | 80.191 | 1.00 | 56.90 | C |
| ATOM | 8843 | C11 | CP3 | C | 900 | −42.026 | −11.334 | 81.861 | 1.00 | 56.30 | C |
| ATOM | 8844 | O10 | CP3 | C | 900 | −40.861 | −10.682 | 82.032 | 1.00 | 50.44 | O |
| ATOM | 8845 | C1 | CP3 | C | 900 | −39.890 | −11.190 | 82.825 | 1.00 | 53.79 | C |
| ATOM | 8846 | C6 | CP3 | C | 900 | −39.819 | −10.900 | 84.267 | 1.00 | 59.69 | C |
| ATOM | 8847 | C8 | CP3 | C | 900 | −40.852 | −10.021 | 84.902 | 1.00 | 57.83 | C |
| ATOM | 8848 | C5 | CP3 | C | 900 | −38.793 | −11.452 | 85.014 | 1.00 | 58.50 | C |
| ATOM | 8849 | C4 | CP3 | C | 900 | −37.836 | −12.268 | 84.410 | 1.00 | 56.00 | C |
| ATOM | 8850 | C7 | CP3 | C | 900 | −36.731 | −12.857 | 85.237 | 1.00 | 52.27 | C |
| ATOM | 8851 | C3 | CP3 | C | 900 | −37.864 | −12.565 | 83.057 | 1.00 | 55.75 | C |
| ATOM | 8852 | C2 | CP3 | C | 900 | −38.856 | −12.054 | 82.245 | 1.00 | 53.15 | C |
| ATOM | 8853 | C9 | CP3 | C | 900 | −38.903 | −12.362 | 80.784 | 1.00 | 46.31 | C |
| ATOM | 8854 | O19 | OLA | C | 901 | −29.771 | −35.705 | 85.385 | 1.00 | 70.12 | O |
| ATOM | 8855 | C18 | OLA | C | 901 | −30.942 | −36.037 | 85.356 | 1.00 | 75.31 | C |
| ATOM | 8856 | O20 | OLA | C | 901 | −31.961 | −35.145 | 85.888 | 1.00 | 71.95 | O |
| ATOM | 8857 | C17 | OLA | C | 901 | −31.347 | −37.374 | 84.771 | 1.00 | 71.01 | C |
| ATOM | 8858 | C16 | OLA | C | 901 | −32.842 | −37.428 | 84.484 | 1.00 | 59.75 | C |
| ATOM | 8859 | C15 | OLA | C | 901 | −33.095 | −37.572 | 82.990 | 1.00 | 57.11 | C |
| ATOM | 8860 | C14 | OLA | C | 901 | −34.584 | −37.649 | 82.671 | 1.00 | 59.97 | C |
| ATOM | 8861 | C13 | OLA | C | 901 | −34.813 | −37.945 | 81.196 | 1.00 | 50.98 | C |
| ATOM | 8862 | C12 | OLA | C | 901 | −35.619 | −36.866 | 80.491 | 1.00 | 42.62 | C |
| ATOM | 8863 | C11 | OLA | C | 901 | −36.292 | −37.372 | 79.216 | 1.00 | 46.62 | C |
| ATOM | 8864 | C10 | OLA | C | 901 | −36.965 | −38.709 | 79.429 | 1.00 | 50.31 | C |
| ATOM | 8865 | C9 | OLA | C | 901 | −38.199 | −38.980 | 79.017 | 1.00 | 48.20 | C |
| ATOM | 8866 | C8 | OLA | C | 901 | −39.128 | −37.920 | 78.486 | 1.00 | 46.46 | C |
| ATOM | 8867 | C7 | OLA | C | 901 | −39.487 | −36.916 | 79.562 | 1.00 | 44.24 | C |
| ATOM | 8868 | C6 | OLA | C | 901 | −40.831 | −36.280 | 79.266 | 1.00 | 48.42 | C |
| ATOM | 8869 | C5 | OLA | C | 901 | −41.672 | −36.178 | 80.531 | 1.00 | 50.35 | C |
| ATOM | 8870 | C4 | OLA | C | 901 | −42.089 | −34.740 | 80.819 | 1.00 | 43.78 | C |
| ATOM | 8871 | C3 | OLA | C | 901 | −43.488 | −34.393 | 80.315 | 1.00 | 44.07 | C |
| ATOM | 8872 | C2 | OLA | C | 901 | −44.338 | −33.767 | 81.418 | 1.00 | 39.71 | C |
| ATOM | 8873 | C1 | OLA | C | 901 | −45.150 | −32.575 | 80.958 | 1.00 | 33.93 | C |
| TER | | | | | | | | | | | |
| ATOM | 8874 | O4 | SO4 | D | 1 | −88.362 | 19.342 | 36.248 | 1.00 | 82.39 | O |
| ATOM | 8875 | S | SO4 | D | 1 | −88.081 | 18.195 | 35.390 | 1.00 | 77.84 | S |
| ATOM | 8876 | O1 | SO4 | D | 1 | −89.035 | 17.128 | 35.680 | 1.00 | 74.85 | O |
| ATOM | 8877 | O2 | SO4 | D | 1 | −86.724 | 17.722 | 35.647 | 1.00 | 70.34 | O |
| ATOM | 8878 | O3 | SO4 | D | 1 | −88.204 | 18.588 | 33.989 | 1.00 | 67.28 | O |
| ATOM | 8879 | O4 | SO4 | D | 2 | −0.406 | 6.729 | 45.987 | 1.00 | 66.31 | O |
| ATOM | 8880 | S | SO4 | D | 2 | 0.649 | 5.726 | 45.868 | 1.00 | 78.50 | S |
| ATOM | 8881 | O1 | SO4 | D | 2 | 1.817 | 6.152 | 46.636 | 1.00 | 78.54 | O |
| ATOM | 8882 | O2 | SO4 | D | 2 | 1.022 | 5.577 | 44.465 | 1.00 | 67.78 | O |
| ATOM | 8883 | O3 | SO4 | D | 2 | 0.167 | 4.445 | 46.376 | 1.00 | 65.21 | O |
| ATOM | 8884 | O4 | SO4 | D | 3 | −85.597 | 10.579 | 39.130 | 1.00 | 55.41 | O |
| ATOM | 8885 | S | SO4 | D | 3 | −86.888 | 10.214 | 38.559 | 1.00 | 52.90 | S |
| ATOM | 8886 | O1 | SO4 | D | 3 | −86.840 | 10.380 | 37.110 | 1.00 | 56.63 | O |
| ATOM | 8887 | O2 | SO4 | D | 3 | −87.924 | 11.075 | 39.122 | 1.00 | 51.41 | O |
| ATOM | 8888 | O3 | SO4 | D | 3 | −87.186 | 8.821 | 38.877 | 1.00 | 43.36 | O |
| ATOM | 8889 | S | SO4 | D | 4 | −19.837 | 5.228 | 33.134 | 1.00 | 96.67 | S |
| ATOM | 8890 | O1 | SO4 | D | 4 | −18.903 | 5.599 | 31.991 | 1.00 | 68.37 | O |
| ATOM | 8891 | O2 | SO4 | D | 4 | −20.836 | 4.213 | 32.596 | 1.00 | 75.98 | O |
| ATOM | 8892 | O3 | SO4 | D | 4 | −19.037 | 4.521 | 34.073 | 1.00 | 75.16 | O |
| ATOM | 8893 | O4 | SO4 | D | 4 | −20.591 | 6.401 | 33.412 | 1.00 | 72.76 | O |
| ATOM | 8894 | S | SO4 | D | 5 | −68.532 | 4.886 | 32.004 | 1.00 | 103.55 | S |
| ATOM | 8895 | O1 | SO4 | D | 5 | −67.305 | 4.886 | 31.103 | 1.00 | 87.86 | O |
| ATOM | 8896 | O2 | SO4 | D | 5 | −69.759 | 4.886 | 31.103 | 1.00 | 81.53 | O |
| ATOM | 8897 | O3 | SO4 | D | 5 | −68.532 | 3.623 | 32.656 | 1.00 | 85.99 | O |
| ATOM | 8898 | O4 | SO4 | D | 5 | −68.532 | 6.149 | 32.656 | 1.00 | 73.29 | O |
| ATOM | 8899 | O | HOH | D | 100 | −6.465 | −2.426 | 20.094 | 1.00 | 49.11 | O |
| ATOM | 8900 | O | HOH | D | 101 | −71.978 | 4.861 | 34.469 | 1.00 | 30.49 | O |
| ATOM | 8901 | O | HOH | D | 102 | −3.612 | −9.252 | 50.187 | 1.00 | 49.72 | O |
| ATOM | 8902 | O | HOH | D | 103 | −9.033 | −0.143 | 35.069 | 1.00 | 39.38 | O |
| ATOM | 8903 | O | HOH | D | 104 | 2.134 | 2.881 | 56.464 | 1.00 | 45.54 | O |
| ATOM | 8904 | O | HOH | D | 105 | −15.922 | 7.149 | 33.316 | 1.00 | 28.15 | O |
| ATOM | 8905 | O | HOH | D | 106 | −24.172 | 5.069 | 32.348 | 1.00 | 40.48 | O |
| ATOM | 8906 | O | HOH | D | 107 | −6.218 | −7.272 | 24.028 | 1.00 | 58.54 | O |
| ATOM | 8907 | O | HOH | D | 108 | −83.790 | 24.658 | 21.727 | 1.00 | 54.82 | O |
| ATOM | 8908 | O | HOH | D | 109 | −75.056 | 27.528 | 22.929 | 1.00 | 37.80 | O |
| ATOM | 8909 | O | HOH | D | 110 | −77.933 | 8.392 | 27.721 | 1.00 | 46.79 | O |
| ATOM | 8910 | O | HOH | D | 111 | −89.858 | 29.924 | 33.913 | 1.00 | 64.11 | O |
| ATOM | 8911 | O | HOH | D | 112 | −67.224 | −2.240 | 23.082 | 1.00 | 37.76 | O |
| ATOM | 8912 | O | HOH | D | 113 | −69.746 | 17.845 | 25.905 | 1.00 | 39.25 | O |
| TER | | | | | | | | | | | |
| END | | | | | | | | | | | |

1. A method of predicting a three dimensional structural representation of a target protein of unknown structure, or part thereof, comprising:
(a) providing the coordinates of the human corticotropin-releasing factor receptor-1 (CRF1R) structure listed in Table A, Table B or Table C, optionally varied by a root mean square deviation of residue backbone atoms of not more than 4.383 Å, or selected coordinates thereof; and
(b) predicting the three-dimensional structural representation of the target protein, or part thereof, by modelling the structural representation on all or the selected coordinates of the CRF1R structure.

2. A method according to claim 1 further comprising aligning the amino acid sequence of the target protein of unknown structure with the amino acid sequence of the CRF1R listed in FIG. 12 to match homologous regions of the amino acid sequences prior to predicting the structural representation, and wherein modelling the structural representation comprises modelling the structural representation of the matched homologous regions of the target protein on the corresponding regions of the CRF1R to obtain a three dimensional structural representation for the target protein that substantially preserves the structural representation of the matched homologous regions.

3. A method according to claim 1, wherein step (b) comprises either (i) positioning the coordinates in the crystal unit cell of the protein so as to predict its structural representation, or (ii) manipulating the coordinates to assign, or account for, peaks in NMR spectra.

4. A method according to claim 1, wherein step (b) comprises
providing an X-ray diffraction pattern of the target protein; and
using the coordinates to predict at least part of the structure coordinates of the target protein.

5. A method according to claim 1, wherein the target protein is a GPCR, such as a Class B GPCR selected from the group consisting of glucagon-like peptide 1 receptor (GLP1R), glucagon-like peptide 2 receptor (GLP2R), calcitonin receptor (CT), amylin/CGRP receptor (AMY₁α), amylin receptor (AMY₂α), amylin/CGRP receptor (AMY₃α), CGRP/adrenomedullin receptor (CGRP₁α), adrenomedullin/CGRP receptor (AM₁α), adrenomedullin/CGRP receptor (AM₂α receptor), corticotropin releasing factor receptor (CRF₁), urocortins receptor (CRF₂), growth hormone releasing hormone receptor (GHRH), gastric inhibitory polypeptide receptor (GIP), glucagon receptor, secretin receptor, TIP-39 receptor (PTH2), parathyroid hormone receptor (PTH1), VIP/PACAP receptor (VPAC₁), PACAP receptor (PAC₂), and VIP/PACAP receptor (VPAC₂).

6. A method for selecting or designing one or more binding partners of a CRF1R comprising using molecular modelling means to select or design one or more binding partners of CRF1R, wherein the three-dimensional structural representation of at least part of the CRF1R, as defined by the coordinates of the human CRF1R listed in Table A or Table B or Table C, optionally varied by a root mean square deviation of residue backbone atoms of not more than 4.383 Å, or selected coordinates thereof, is compared with a three-dimensional structural representation of one or more candidate binding partners, and one or more binding partners that are predicted to interact with CRF1R are selected.

7. A method according to claim 6, the CRF1R having a binding pocket in the position structurally equivalent to the binding pocket of human CRF1R that is defined by residues including (a) Leu 158, Phe 162, His 199, Asn 202, Phe203, Phe 204, Trp205, Met 206, Phe 207, Gly 208, Glu 209, Gly 210, Cys211, Leu 213, His 214, Met 276, Val 279 Leu 280, Leu 281, Ile 282, Asn 283, Phe 284, Ile 285, Phe 286, Leu 287, Phe 288, Ile 290, Ala 312, Ala 315, Thr 316, Leu 317, Leu 319, Leu 320, Pro 321, Leu323, Gly 324, Ile 325, Tyr 327, Gln 355, Val 359 and Phe 362 of human CRF1R or (b) Ala119, Asn123, His127, Ser130, Phe162, Arg165, Asn166, Thr168, Thr169, Val172, Gln173, Thr175, Met176, His181, Val191, Thr192, Tyr195, Asn196, His199, Asn202, Phe203, Lys257, Ala260, Lys262, Tyr272, Gln273, Met276, Leu323, Thr326, Tyr327, Ala330, Phe331, Asn333, Asp337, Arg341, Phe344, Ile345, Asn348, Glu352, Ser353 and Gln355 of human CRF1R, the method further comprising the step of using molecular modelling means to select or design one or more binding partners that are predicted to interact with the said CRF1R, wherein a three-dimensional structural representation of one or more candidate binding partners are compared with a three-dimensional structural representation of the said binding pocket, and one or more candidate binding partners that are predicted to interact with the said binding pocket, are selected or designed.

8. A method according to claim 7, wherein the three-dimensional structural representation is that defined by the coordinates listed in Table A or Table B or Table C, optionally varied by a root mean square deviation of residue backbone atoms of not more than 4.383 Å, or selected coordinates thereof.

9. A method for producing a binding partner of CRF1R comprising:
identifying a binding partner according to the method of claim 6, and
synthesising the binding partner.

10. A binding partner produced by the method of claim 9.

11.-13. (canceled)

14. A method for producing a medicament, pharmaceutical composition or drug, the process comprising: (a) providing a binding partner according to claim 10 and (b) preparing a medicament, pharmaceutical composition or drug containing the binding partner.

15.-16. (canceled)

17. A computer system for use in the method according to claim 1, the system containing computer-readable data comprising one or more of:
(a) the coordinates of the human CRF1R structure, listed in Table A or Table B or Table C, optionally varied by a root mean square deviation of residue backbone atoms of not more than 4.383 Å, or selected coordinates thereof;
(b) the coordinates of a target CRF1R homologue or analogue generated by homology modelling of the target based on the data in (a);
(c) the coordinates of a binding partner generated by interpreting X-ray crystallographic data or NMR data by reference to the coordinates of the human CRF1R structure, listed in Table A or Table B or Table C, optionally varied by a root mean square deviation of residue backbone atoms of not more than 4.383 Å, and
(d) structure factor data derivable from the coordinates of (a), (b) or (c).

18. A computer-readable storage medium for use in the method according to claim 1, comprising a data storage material encoded with computer readable data, wherein the data comprises one or more of
(a) the coordinates of the human CRF1R structure, listed in Table A or Table B or Table C, optionally varied by a root mean square deviation of residue backbone atoms of not more than 4.383 Å, or selected coordinates thereof;
(b) the coordinates of a target CRF1R receptor homologue or analogue generated by homology modelling of the target based on the data in (a);
(c) the coordinates of a binding partner generated by interpreting X-ray crystallographic data or NMR data by reference to the coordinates of the human CRF1R structure, listed in Table A or Table B or Table C, optionally varied by a root mean square deviation of residue backbone atoms of not more than 4.383 Å, or selected coordinates thereof, and
(d) structure factor data derivable from the coordinates of (a), (b) or (c).

19. A computer-readable storage medium for use in the method according to claim 1, comprising a data storage material encoded with a first set of computer-readable data comprising a Fourier transform of at least a portion of the structural coordinates of human CRF1R listed in Table A or Table B or Table C, optionally varied by a root mean square deviation of residue backbone atoms of not more than 4.383 Å, or selected coordinates thereof; which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

20.-25. (canceled)

26. A crystal of CRF1A having the structure defined by the coordinates of the human CRF1R structure, listed in Table A or Table B or Table C, optionally varied by a root mean square deviation of residue backbone atoms of not more than 4.383 Å, or selected coordinates thereof.

27. A crystal according to claim 26, which has P22₁2₁ symmetry and unit cell dimensions a=86.6 (±15) Å, b=124.0 (±15) Å, c=166.8 (±15) Å.

28. (canceled)

29. A crystal according to claim 26 having a resolution of 3.15 Å or better.


SEQUENCE LISTING (70 sequences)

SEQ ID NO: 1 - protein, 117 aa, Homo sapiens
MEILNEEKKSKVHYHVAAIINYLGHCISLVALLVAFVLFLRARSIRCLRNIIHANLIAAFILRNATWFVVQLTMSPEVHSNVGWCRLVTAAYNYFHVTNFFWMFGEGCYLHTAIVLT

SEQ ID NO: 2 - protein, 165 aa, Homo sapiens
LTDRLRAWMFICIGWGVPFPIIVAWAIGKLYYDNEKCWAGKRPGVYTDYIYQGPMALVLLINFIFLFNIVRILMTKLRASTTSETIQARKAVKATLVLLPLLGITYMLAFVNPGEDEVSRVVFIYFNAFLESFQGFFVSVFACFLNSEVRSAAAAHHHHHHHHHH

SEQ ID NO: 3 - protein, 163 aa, homo sapien
DRLRAWMFICIGWGVPFPIIVAWAIGKLYYDNEKCWAGKRPGVYTDYIYQGPMALVLLINFIFLFNIVRILMTKLRASTTSETIQARKAVKATLVLLPLLGITYMLAFVNPGEDEVSRVVFIYFNAFLESFQGFFVSVFACFLNSEVRSAAAAHHHHHHHHHH

SEQ ID NO: 4 - protein, 14 aa, Homo sapiens
FMVLNFLITLLLGY

SEQ ID NO: 5 - protein, 14 aa, Homo sapiens
YMALNFLITLVLGF

SEQ ID NO: 6 - protein, 14 aa, Homo sapiens
YMALNLLITLVLGF

SEQ ID NO: 7 - protein, 14 aa, Homo sapiens
YLAINFFVTLILGE

SEQ ID NO: 8 - protein, 14 aa, Homo sapiens
YLAINFFITLILGE

SEQ ID NO: 9 - protein, 14 aa, Homo sapiens
YLTINFFITLVLGE

SEQ ID NO: 10 - protein, 14 aa, Homo sapiens
YLCVNFFITLILGE

SEQ ID NO: 11 - protein, 14 aa, Homo sapiens
YLSINFFITLIFGY

SEQ ID NO: 12 - protein, 14 aa, Homo sapiens
FLSINFFITLIFGY

SEQ ID NO: 13 - protein, 14 aa, Homo sapiens
YLSINFFITLIFGY

SEQ ID NO: 14 - protein, 14 aa, Homo sapiens
FLSVNFFITLIFGY

SEQ ID NO: 15 - protein, 14 aa, homo sapien
YISINFFITLMFGY

SEQ ID NO: 16 - protein, 14 aa, HOMO SAPIENS
YIAINFFTTLVFGY

SEQ ID NO: 17 - DNA, 1338 bp, Artificial sequence
  synthetic polynucleotide
atgggcgggcacccccagctgagactggtcaaagcactgctgctgctgggcctgaaccctgtcagcgcttccctgcaagaccaacattgcgagagcctgtccctggcaagtaatatcagcggtctgcaatgtaacgcctccgtggatctgatcggaacttgttggccccgaagcccagccggacaactggtcgtccgaccttgtcccgcattcttctacggagtgcggtataatactacaaacaatggttaccgcgaatgtctggcaaacggctcatgggcagcacgagtgaattattccgagtgtcaggaaatcctgaacgaggaaaagaagagcaaagtgcactaccacgtcgccgtgattatcaattatctggggcactgcattagtctggtcgctctgctggtcgccttcgtgctgtttctgcgcctgcggtctatccgctgtctgcgaaacattatccactggaacctgatttccgccttcattctgagaaacgctacctggtttgtggtccaactgaccatgtcacccgaggtgcaccagagtaacgtgggctggtgcaggctggtgacagcagcctataattacttccatgtcaccaatttcttctggatgtttggcgaaggttgctatctgcatacagccattgtgctgacttattccaccgaccggctgcgcaagtggatgttcatctgcattgggtggggagtgccttttccaattatcgtcgcttgggcaatcggcaaactgtactatgataacgagaagtgttggttcggcaagcgaccaggggtgtacacagactatatctatcaaggacctatgatcctggtgctgctgattaacttcattttcctgtttaacatcgtccgcattctgatgactaagctgcgggcctctaccactagtgaaaccatccagtatcgaaaagcagtgaaggctaccctggtcctgctgcctctgctgggtattacttatatgctgttcttcgtcaatccaggggaagatgaagtctcacgcgtggtctttatctactttaacagcttcctggagtcctttcaaggcttctttgtcagcgtgttctactgctttctgaatagcgaggtcagatcagctattaggaaacgatggcatcgatggcaggacaagcacagtatccgagctagagtggctagggctatgagtattcctacctctccaactcgggtgagcttccatagtattaaacagtcaacagctgtcgcggccgcagagaacctgtacttccagagcgagcagaagctgatcagcgaagaggacctgcaccaccaccatcaccatcatcaccaccattaa

SEQ ID NO: 18 - protein, 445 aa, artificial sequence
  synthetic polypeptide
MGGHPQLRLVKALLLLGLNPVSASLQDQHCESLSLASNISGLQCNASVDLIGTCWPRSPAGQLVVRPCPAFFYGVRYNTTNNGYRECLANGSWAARVNYSECQEILNEEKKSKVHYHVAVIINYLGHCISLVALLVAFVLFLRLRSIRCLRNIIHWNLISAFILRNATWFVVQLTMSPEVHQSNVGWCRLVTAAYNYFHVTNFFWMFGEGCYLHTAIVLTYSTDRLRKWMFICIGWGVPFPIIVAWAIGKLYYDNEKCWFGKRPGVYTDYIYQGPMILVLLINFIFLFNIVRILMTKLRASTTSETIQYRKAVKATLVLLPLLGITYMLFFVNPGEDEVSRVVFIYFNSFLESFQGFFVSVFYCFLNSEVRSAIRKRWHRWQDKHSIRARVARAMSIPTSPTRVSFHSIKQSTAVAAAENLYFQSEQKLISEEDLHHHHHHHHHH

SEQ ID NO: 19 - DNA, 1338 bp, artificial sequence
  synthetic polynucleotide
atgggcgggcacccccagctgagactggtcaaagcactgctgctgctgggcctgaaccctgtcagcgcttccctgcaagaccaacattgcgagagcctgtccctggcaagtaatatcagcggtctgcaatgtaacgcctccgtggatctgatcggaacttgttggccccgaagcccagccggacaactggtcgtccgaccttgtcccgcattcttctacggagtgcggtataatactacaaacaatggttaccgcgaatgtctggcaaacggctcatgggcagcacgagtgaattattccgagtgtcaggaaatcctgaacgaggaaaagaagagcaaagtgcactaccacgtcgccgctattatcaattatctggggcactgcattagtctggtcgctctgctggtcgccttcgtgctgtttctgcgcgctcggtctatccgctgtctgcgaaacattatccacgctaacctgattgctgccttcattctgagaaacgctacctggtttgtggtccaactgaccatgtcacccgaggtgcaccagagtaacgtgggctggtgcaggctggtgacagcagcctataattacttccatgtcaccaatttcttctggatgtttggcgaaggttgctatctgcatacagccattgtgctgacttatgctaccgaccggctgcgcgcttggatgttcatctgcattgggtggggagtgccttttccaattatcgtcgcttgggcaatcggcaaactgtactatgataacgagaagtgttgggctggcaagcgaccaggggtgtacacagactatatctatcaaggacctatggccctggtgctgctgattaacttcattttcctgtttaacatcgtccgcattctgatgactaagctgcgggcctctaccactagtgaaaccatccaggcacgaaaagcagtgaaggctaccctggtcctgctgcctctgctgggtattacttatatgctggctttcgtcaatccaggggaagatgaagtctcacgcgtggtctttatctactttaacgccttcctggagtcctttcaaggcttctttgtcagcgtgttcgcctgctttctgaatagcgaggtcagatcagctattaggaaacgatggcatcgatggcaggacaagcacagtatccgagctagagtggctagggctatgagtattcctacctctccaactcgggtgagcttccatagtattaaacagtcaacagctgtcgcggccgcagaaaatctgtattttcagagcgaacagaaactgattagcgaagaagatctgcaccatcaccatcaccatcaccatcaccattaa

SEQ ID NO: 20 - protein, 445 aa, artificial sequence
  synthetic polypeptide
MGGHPQLRLVKALLLLGLNPVSASLQDQHCESLSLASNISGLQCNASVDLIGTCWPRSPAGQLVVRPCPAFFYGVRYNTTNNGYRECLANGSWAARVNYSECQEILNEEKKSKVHYHVAAIINYLGHCISLVALLVAFVLFLRARSIRCLRNIIHANLIAAFILRNATWFVVQLTMSPEVHQSNVGWCRLVTAAYNYFHVTNFFWMFGEGCYLHTAIVLTYATDRLRAWMFICIGWGVPFPIIVAWAIGKLYYDNEKCWAGKRPGVYTDYIYQGPMALVLLINFIFLFNIVRILMTKLRASTTSETIQARKAVKATLVLLPLLGITYMLAFVNPGEDEVSRVVFIYFNAFLESFQGFFVSVFACFLNSEVRSAIRKRWHRWQDKHSIRARVARAMSIPTSPTRVSFHSIKQSTAVAAAENLYFQSEQKLISEEDLHHHHHHHHHH

SEQ ID NO: 21 - protein, 29 aa, Homo sapiens
HYHVAVIINYLGHCISLVALLVAFVLFLR

SEQ ID NO: 22 - protein, 29 aa, homo sapiens
HYRIALVVNYLGHCVSVAALVAAFLLFLA

SEQ ID NO: 23 - protein, 29 aa, homo sapiens
NAYVLYYLAIVGHSLSIFTLVISLGIFVF

SEQ ID NO: 24 - protein, 29 aa, homo sapiens
TALNLFYLTIIGHGLSIASLLISLGIFFY

SEQ ID NO: 25 - protein, 29 aa, homo sapiens
QLLFLYIIYTVGYALSFSALVIASAILLG

SEQ ID NO: 26 - protein, 29 aa, homo sapiens
LLSTLQLMYTVGYSFSLISLFLALTLLLF

SEQ ID NO: 27 - protein, 29 aa, homo sapiens
MYSSFQVMYTVGYSLSLGALLLALAILGG

SEQ ID NO: 28 - protein, 29 aa, homo sapiens
YLLKLKVMYTVGYSSSLVMLLVALGILCA

SEQ ID NO: 29 - protein, 29 aa, homo sapiens
VFDRLGMIYTVGYSVSLASLTVAVLILAY

SEQ ID NO: 30 - protein, 29 aa, homo sapiens
FFERLYVMYTVGYSISFGSLAVAILIIGY

SEQ ID NO: 31 - protein, 27 aa, homo sapiens
LRNIIHWNLISAFILRNATWFVVQLTM

SEQ ID NO: 32 - protein, 27 aa, homo sapiens
LRNVIHWNLITTFILRNVMWFLLQLVD

SEQ ID NO: 33 - protein, 27 aa, homo sapiens
QRVTLHKNMFLTYILNSMIIIIHLVEV

SEQ ID NO: 34 - protein, 27 aa, homo sapiens
QRITLHKNLFFSFVCNSVVTIIHLTAV

SEQ ID NO: 35 - protein, 27 aa, homo sapiens
TRNYIHLNLFASFILRALSVFIKDAAL

SEQ ID NO: 36 - protein, 27 aa, homo sapiens
TRNYIHMNLFASFILRTLAVLVKDVVF

SEQ ID NO: 37 - protein, 27 aa, homo sapiens
TRNAIHANLFASFVLKASSVLVIDGLL

SEQ ID NO: 38 - protein, 27 aa, homo sapiens
TRNYIHMHLFVSFILRALSNFIKDAVL

SEQ ID NO: 39 - protein, 27 aa, homo sapiens
TRNYIHMHLFLSFMLRAVSIFVKDAVL

SEQ ID NO: 40 - protein, 27 aa, homo sapiens
TRNYIHMHLFVSFMLRATSIFVKDRVV

SEQ ID NO: 41 - protein, 30 aa, homo sapiens
DRLRKWMFICIGWGVPFPIIVAWAIGKLYY

SEQ ID NO: 42 - protein, 30 aa, homo sapiens
ERLRKCLFLFIGWCIPFPIIVAWAIGKLYY

SEQ ID NO: 43 - protein, 30 aa, homo sapiens
EKQRLRWYYLLGWGFPLVPTTIHAITRAVY

SEQ ID NO: 44 - protein, 30 aa, Homo sapiens
EKQHLMWYYFLGWGFPLIPACIHAIARSLY

SEQ ID NO: 45 - protein, 30 aa, homo sapiens
EQWIFRLYVSIGWGVPLLFVVPWGIVKYLY

SEQ ID NO: 46 - protein, 30 aa, homo sapiens
ERRLWPRYLLLGWAFPVLFVVPWGFARAHL

SEQ ID NO: 47 - protein, 30 aa, homo sapien
ERSFFSLYLGIGWGAPMLFVVPWAVVKCLF

SEQ ID NO: 48 - protein, 30 aa, homo sapiens
ERKYLQGFVAFGWGSPAIFVALWAIARHFL

SEQ ID NO: 49 - protein, 30 aa, homo sapiens
EKKYLWGFTVFGWGLPAVFVAVWVSVRATL

SEQ ID NO: 50 - protein, 30 aa, homo sapiens
DTKYLWGFILIGWGFPAAFVAAWAVARATL

SEQ ID NO: 51 - protein, 30 aa, homo sapien
VSRVVFIYFNSFLESFQGFFVSVFYCFLNS

SEQ ID NO: 52 - protein, 30 aa, homo sapiens
LSQIMFIYFNSFLQSFQGFFVSVGYCFFNG

SEQ ID NO: 53 - protein, 30 aa, homo sapiens
MLGKIYDYVMHSLIHFQGFFVATIYCFCNN

SEQ ID NO: 54 - protein, 30 aa, homo sapiens
IAEEVYDYIMHILMHFQGLLVSTIFCFFNG

SEQ ID NO: 55 - protein, 30 aa, homo sapiens
TLRFIKLFTELSFTSFQGLMVAILYCFVNN

SEQ ID NO: 56 - protein, 30 aa, homo sapiens
FAKLIRLFIQLTLSSFHGFLVALQYGFANG

SEQ ID NO: 57 - protein, 30 aa, homo sapiens
TLRSAKLFFDLFLSSFQGLLVAVLYCFLNK

SEQ ID NO: 58 - protein, 30 aa, homo sapiens
DAMEIQLFFELALGSFQGLVVAVLYCFLNG

SEQ ID NO: 59 - protein, 30 aa, homo sapiens
TLWQVQMHYEMLFNSFQGFFVAIIYCFCNG

SEQ ID NO: 60 - protein, 30 aa, homo sapiens
LGWEIRMHCELFFNSFQGFFVSIIYCYCNG

SEQ ID NO: 61 - protein, 36 aa, homo sapiens
VGWCRLVTAAYNYFHVTNFFWMFGEGCYLHTAIVLT

SEQ ID NO: 62 - protein, 36 aa, homo sapiens
EVWCRCITTIFNYFVVTNFFWMFVEGCYLHTAIVMT

SEQ ID NO: 63 - protein, 36 aa, homo sapiens
PVSCKILHFFHQYMMACNYFWMLCEGIYLHTLIVVA

SEQ ID NO: 64 - protein, 36 aa, homo sapiens
PVSCKVSQFIHLYLMGCNYFWMLCEGIYLHTLIVVA

SEQ ID NO: 65 - protein, 36 aa, homo sapiens
SLSCRLVFLLMQYCVAANYYWLLVEGVYLYTLLAFS

SEQ ID NO: 66 - protein, 35 aa, homo sapiens
STSCRSVQLLHYFVGANYLWLLVEGLYLHTLLEPT

SEQ ID NO: 67 - protein, 36 aa, homo sapiens
VAGCRVAAVFMQYGIVANYCWLLVEGLYLHNLLGLA

SEQ ID NO: 68 - protein, 36 aa, homo sapiens
RAGCKLVMVLFQYCIMANYSWLLVEGLYLHTLLAIS

SEQ ID NO: 69 - protein, 36 aa, homo sapiens
YAGCRVAVTFFLYFLATNYYWILVEGLYLHSLIFMA

SEQ ID NO: 70 - protein, 36 aa, homo sapiens
YIGCKIAVVMFIYFLATNYYWILVEGLYLHNLIFVA